(19) United States
(12) Patent Application Publication
(10) Pub. No.: US 2024/0150368 A1
(43) Pub. Date: May 9, 2024

(54) ULK INHIBITORS AND METHODS OF USE THEREOF

(71) Applicant: Deciphera Pharmaceuticals, LLC, Waltham, MA (US)

(72) Inventors: Justin T. Proto, Tonganoxie, KS (US); Andrew Michael Harned, Lawrence, KS (US); Kristen Stoltz, Waltham, MA (US); Timothy Caldwell, Lawrence, KS (US); Lakshminarayana Vogeti, Waltham, MA (US); Yu Mi Ahn, Waltham, MA (US); Bertrand Le Bourdonnec, Waltham, MA (US); Daniel L. Flynn, Waltham, MA (US)

(21) Appl. No.: 18/456,831

(22) Filed: Aug. 28, 2023

Related U.S. Application Data
(60) Provisional application No. 63/403,417, filed on Sep. 2, 2022.

(51) Int. Cl. C07D 487/10; A61K 45/06; C07D 209/34; C07D 401/14; C07D 403/12; C07D 405/14; C07D 413/12; C07D 487/04; C07D 487/08; C07D 491/107

(52) U.S. Cl. CPC C07D 487/10; A61K 45/06; C07D 209/34; C07D 401/14; C07D 403/12; C07D 405/14; C07D 413/12; C07D 487/04; C07D 487/08; C07D 491/107

(57) ABSTRACT

Described herein are compounds that are inhibitors of autophagy and their use in the treatment of disorders such as cancers.

### CROSS-REFERENCE

This application claims priority to U.S. Provisional Application No. 63/403,417, filed Sep. 2, 2022, the content of which is incorporated herein by reference in its entirety.

### REFERENCE TO A SEQUENCE LISTING

This application contains a sequence listing which has been submitted electronically in XML format. The Sequence Listing XML is incorporated herein by reference. Said XML file, created on Aug. 25, 2023, is named DCP-114 SL.xml and is 9.7 kilobytes in size.

### BACKGROUND

Autophagy (literally meaning “self-eating”) is a process that enables cells to recycle cellular organelles, proteins, stored lipids, glucagon, and other materials for the purpose of generating nutrients under periods of stress. These cellular contents are recycled by engulfment in vesicles called autophagosomes. Autophagosomes subsequently merge with lysosomes that degrade the autophagosomal contents for recycling of nutrients to the cell. Tumor cells are prone to activate autophagy, as these cells have a high metabolic demand, experience cellular stress, and frequently are in hypoxic environments with limited blood flow and nutrient supply. Moreover, chemotherapy and targeted anti-cancer therapies have been shown to induce autophagy as a treatment resistance mechanism, and combination of autophagy inhibition (by genetic loss of function mutations in autophagy genes or by pharmacologic means) with chemotherapeutic regimens has been shown to suppress tumor growth and trigger tumor cell apoptosis to a greater extent than single agent chemotherapy.

Mutant RTK, RAS, and RAF proteins drive approximately 70 percent of all human cancers—including 95 percent of pancreatic cancers and 45 percent of colorectal cancers, and treatment of these mutant RTK/RAS/RAF cancers is currently an area of high unmet medical need. These cancers are highly proliferative and depend on basal levels of autophagy for survival, suggesting that inhibition of autophagy in these “autophagy addicted” cancers is a viable therapeutic approach.

ULK1 kinase is the initiating protein of autophagy and is a serine/threonine kinase. The ULK1 kinase complex is activated in response to cellular stress including nutrient deprivation and energy depletion. Nutrient deprivation activates ULK kinase activity through inhibition of mTORC1, and energy depletion activates ULK kinase activity through activation by AMP-activated protein kinase AMPK. Importantly, kinase dead mutants of ULK kinase block initiation of canonical autophagy, suggesting that small molecule inhibitors of ULK kinase activity would be able to block autophagy.

Further mechanistic studies have shown that genetic deletion of ULK1 inhibits autophagy in cancer cells, relieving FOX3A turn-over and upregulation of the pro-apoptotic protein PUMA. In addition to classical activation of canonical autophagy, ULK1 kinase activity has been shown to be required for Bcl-2-L-13 mediated mitophagy (autophagy of damaged mitochondria).

ULK1 and ULK2 kinases have also been demonstrated to rewire cancer cell glucose metabolism.

Autophagy is also upregulated in host cells and tissues in cancer. Autophagy in pancreatic tissue stellate cells was demonstrated to support tumor growth. Pancreatic stellate cells were shown to support pancreatic cancer tumor metabolism through autophagic alanine secretion. Inhibition of host tissue autophagy was demonstrated to lead to a depletion in circulating arginine (a required amino acid for tumor metabolism and growth) through liver-mediated increases in arginase secretion. Activation of ULK1 kinase was also shown to inactivate the STING pathway in immune cells through inhibitory phosphorylation of STING, mediating a negative feedback mechanism for limiting an innate immune cell response mediated by interferons. Thus, not only is autophagy activated in tumor cells (cancer cell autonomous), but also in other cells in the tumor microenvironment or host tissues (cancer call nonautonomous) to support tumor survival and growth.

It has been demonstrated that inhibition of receptor tyrosine kinases (RTK) such as EGFR can promote autophagy through signaling via the EGFR→Ras→RAF→MEK→ERK pathway in several cancers, including glioblastoma, human vulvar squamous carcinoma, colorectal adenocarcinoma, and non-small-cell lung cancers. EGFR can also suppress autophagy via the EGFR→PI3K→AKT1→mTORC1 pathway by activating the mTORC1 complex to inactivate downstream ULK1. It has been demonstrated that pharmacological inhibition of either PI3K with LY294002 or mTOR with rapamycin promotes expression of autophagy-related proteins LC3-II/I, p62, and beclin-1, which provides additional rationale for combining such targeted therapeutics or chemotherapeutic agents with inhibitors of autophagy.

Mutant Ras cancers are addicted to autophagy. In pancreatic cancer, mutant Ras signals predominantly through the MAPKAP pathway. Mutant Ras activates RAF kinases, which in turn activate MEK kinases, which finally activate ERK kinases: mutant Ras→RAF→MEK→ERK. Despite mutant Ras signaling through the MAPKAP pathway, inhibitors of this pathway have provided no or little clinical benefit in clinical trials when used as single agents. It has been recently reported that inhibition of the MAPKAP pathway induces autophagy as a compensatory survival mechanism. When MEK inhibitors were combined with the autophagy inhibitor hydroxychloroquine, there was synergistic activity leading to regression of a number of mutant Ras or mutant BRAF cancers. Similarly, when ERK inhibitors were combined with the autophagy inhibitor hydroxychloroquine or chloroquine, there was synergistic activity leading to inhibition of mutant Ras pancreatic cancers. It has been demonstrated that genetic depletion of RAF kinases (CRAF and BRAF) led to synergistic anti-tumor activity in mutant Ras cancer cell lines when autophagy was also genetically depleted.

ULK1/2 inhibitors have the potential to be a promising treatment regimen for patients with mutant Ras cancers when used in combination with RTK inhibitors, MAPKAP pathway inhibitors, PI3K/AKT pathway inhibitors, chemotherapeutic agents, and/or other targeted therapeutics

Mutations in the gene encoding LRRK2 kinase are causative of Parkinson's disease. LRRK2 point mutations are found in both familial (inherited) as well as sporadic Parkinson's disease patients. The most common mutation of LRRK2 in Parkinson's disease is LRRK2 G2019S. These mutations in LRRK2 are gain-of-function mutations that cause overactivation of LRRK2 signaling. Ongoing autophagy is a process that is used by brain neuronal cells to maintain health and homeostasis. Autophagy is a process by which cells identify, localize, and destroy aged organelles and structural elements within cells, and particularly in the case of proteins known to aggregate in neurons, autophagy eliminates such toxic protein aggregates to maintain neuronal health. LRRK2 activity suppresses autophagy, and the LRRK2 G2019S gain-of-function mutant even more so suppresses autophagy and has been linked to aggressive forms of Parkinson's disease.

Increased LRRK2 kinase activity has also been linked to immunoinflammatory diseases including colitis and Crohn's disease and inflammatory bowel disease. In the gastrointestinal tract, LRRK2 is present in antigen-presenting cells including dendritic cells. LRRK2 activity has been shown to be important in Dectin-1 mediated innate immune responses, including an activation of the NFkB pathway and increased TNF-alpha production in dendritic cells of patients with Crohn's disease.

Inhibitors of LRRK2 are sought for the treatment of neurodegenerative diseases including Parkinson's disease, and also are sought for the treatment of gastrointestinal diseases including Crohn's disease, ulcerative colitis, and inflammatory bowel disease.

### SUMMARY

Described herein are compounds that are inhibitors of autophagy, pharmaceutical compositions, and their use as agents in the treatment of disorders such as cancer, and processes for their preparation and pharmaceutical compositions containing them as an active ingredient. Such pharmaceutical compositions may comprise the compound described herein as the sole active agent or in combination with other active agents in the presence of a pharmaceutically acceptable excipient. In an embodiment, the described compounds are inhibitors of ULK kinase activity, including ULK1 and ULK2 activity.

For example, compounds described herein may be represented by Formula I:

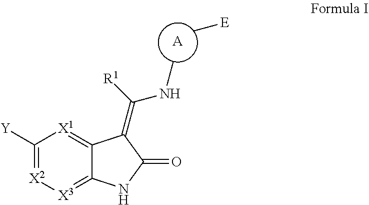

or a pharmaceutically acceptable salt, enantiomer, stereoisomer, or tautomer thereof, wherein: X¹ and X³ are each independently selected from the group consisting of N, CH, and CF; X² is selected from the group consisting of N, CH, CF, and C-Cl; provided that not more than two of X¹, X², and X³ are N; A is selected from the group consisting of optionally substituted heteroaryl wherein the optionally substituted substituent, at each occurrence, is independently selected from the group consisting of alkyl, alkoxy, halogen, haloalkyl, haloalkoxy, hydroxy, and cyano, and optionally substituted phenyl wherein the optionally substituted substituent, at each occurrence, is independently selected from the group consisting of alkyl, alkoxy, halogen, haloalkyl, haloalkoxy, hydroxy, and cyano; E is selected from the group consisting of H, haloalkyl, optionally substituted alkyl wherein the optionally substituted substituent, at each occurrence, is independently selected from the group consisting of amine, alkoxy, halogen, haloalkyl, haloalkoxy, hydroxy, and cyano, optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl; optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl; Y is selected from the group consisting of

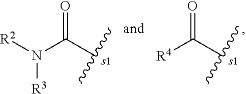

wherein s1 is the site covalently linked to the ring; R¹ is alkyl or haloalkyl; R² and R³ are each independently selected from the group consisting of H, alkoxy, haloalkyl, optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl, optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano, optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano, optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano, provided that when one of R² and R³ is H, the other of R² and R³ is not H, or when Y is

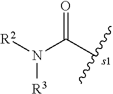

R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano; and R⁴ is selected from the group consisting of optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl, optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano, optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano; provided that when X¹, X², and X³ are CH, Y is

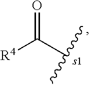

R⁴ is C₁₋₅ alkyl optionally substituted by one or more fluorine atoms, and A is an optionally substituted phenyl, E is not alkyl substituted with amine.

In another embodiment, described herein is a pharmaceutical composition comprising a compound described herein (e.g., a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, enantiomer, stereoisomer, or tautomer thereof, as described herein) and a pharmaceutically acceptable carrier or excipient.

In another embodiment, provided herein is a method of treating a tumor in a patient in need thereof, comprising administering to the patient a therapeutically effective amount of a compound described herein or pharmaceutically acceptable salts, enantiomers, stereoisomers, or tautomers thereof, or of a pharmaceutical composition described herein.

In another embodiment, provided herein is a method of treating a cancer in a patient in need thereof, comprising administering to the patient a therapeutically effective amount of a compound described herein or pharmaceutically acceptable salts, enantiomers, stereoisomers, or tautomers thereof, or of a pharmaceutical composition described herein.

In another embodiment, provided herein is a method of treating a disorder selected from the group consisting of gastrointestinal stromal tumors, esophageal cancer, gastric cancer, melanomas, gliomas, glioblastomas, ovarian cancer, bladder cancer, pancreatic cancer, prostate cancer, lung cancers, breast cancers, renal cancers, hepatic cancers, osteosarcomas, multiple myelomas, leukemias, cervical carcinomas, cancers that are metastatic to bone, papillary thyroid carcinoma, non-small cell lung cancer, and colorectal cancers in a patient in need thereof, comprising administering to the patient a therapeutically effective amount of a compound described herein or pharmaceutically acceptable salts, enantiomers, stereoisomers, or tautomers thereof, or of a pharmaceutical composition described herein.

In some embodiments, the method further comprises administering to the patient one or more additional therapeutic agents.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The definitions set forth in this application are intended to clarify terms used throughout this application.

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used herein, the singular forms “a,” “an,” and, “the” encompass plural references unless the context clearly indicates otherwise. The terms “comprising,” “including,” and “having” are intended to be inclusive and mean that there may be additional elements other than the listed elements.

As used herein, the following definitions shall apply unless otherwise indicated. For purposes of this disclosure, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March's Advanced Organic Chemistry,” 5th Ed., Smith, M. B. and March, J., eds. John Wiley & Sons, New York: 2001, the entire contents of which are hereby incorporated by reference.

As used herein, the term “herein” means the entire application.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen atoms in a given structure with the radical of a specified substituent including, but not limited to, hydroxyl, hydroxyalkyl, alkoxy, halogen, alkyl, cycloalkyl, heterocyclyl, aryl, heteroaryl, amino, aminoalkyl, cyano, haloalkyl, and haloalkoxy. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched, and unbranched, carbocyclic, and heterocyclic, aromatic, and non-aromatic substituents of organic compounds. The permissible substituents can be one or more and the same or different for appropriate organic compounds. For purposes of this application, the heteroatoms such as nitrogen may have hydrogen substituents and/or any permissible substituents of organic compounds described herein which satisfy the valences of the heteroatoms.

Substituents can include any substituents described herein, for example, such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, an alkoxy, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthiols, carbonyls (including ketones, aldehydes, carboxylates, and esters), —CF₃, —CN, and the like. Unless specifically stated as “unsubstituted,” references to chemical moieties herein are understood to include substituted variants. For example, reference to an “aryl” group or moiety implicitly includes both substituted and unsubstituted variants.

As used herein, the term “acyl” refers to a group —C(═O)—R^{w} wherein R^{w} is optionally substituted alkyl. Examples of “acyl” include, but are not limited to, instances where R^{w} is C₁-C₁₀ alkyl (C₁-C₁₀ acyl) or C₁-C₆-alkyl (C₁-C₆ acyl). In some embodiments, each occurrence of the optionally substituted substituent is independently selected from the group consisting of H, OH, alkoxy, cyano, F, and amino. Additional examples of “acyl” include —C(═O)—CH₃, —C(═O)—CH₂—CH₃, —C(═O)—CH₂—CH₂—CH₃, or —C(═O)—CH(CH₃)₂.

As used herein, the term “alkyl” refers to a straight chained or branched non-aromatic hydrocarbon which is completely saturated. Typically, a straight chained or branched alkyl group has from 1 to about 20 carbon atoms, preferably from 1 to about 10, e.g., may be C₁-C₆ alkyl or e.g., C₁-C₆ alkyl unless otherwise defined. Examples of straight chained and branched alkyl groups include, but are not limited to, methyl, ethyl, 1-propyl (n-propyl), 2-propyl, n-butyl, sec-butyl, tertbutyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl, or 4-octyl, and the like. Moreover, the term “alkyl” used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. The “alkyl” group may be optionally substituted.

The term “Cₓ-C_{y}” when used in conjunction with a chemical moiety, such as, acyl, acyloxy, alkyl, alkenyl, or alkoxy is meant to include groups that contain from x to y carbons in the chain. For example, the term “Cₓ-C_{y}” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal.

As used herein, the term “alkoxy” refers to a straight or branched, saturated aliphatic (alkyl) hydrocarbon radical bonded to an oxygen atom that is attached to a core structure. Preferably, alkoxy groups have one to six carbon atoms, i.e., may be C₁-C₆ alkoxy. Examples of alkoxy groups include but are not limited to methoxy, ethoxy, propoxy, isopropoxy, butoxy, isobutoxy, tert-butoxy, pentoxy, 3-methyl butoxy and the like.

As used herein, the term “haloalkyl” refers to alkyl group (as defined above) is substituted with one or more halogens. A monohaloalkyl radical, for example, may have a chlorine, bromine, iodine, or fluorine atom. Dihalo and polyhaloalkyl radicals may have two or more of the same or different halogen atoms. Examples of haloalkyl include, but are not limited to, chloromethyl, dichloromethyl, trichloromethyl, dichloroethyl, dichloropropyl, fluoromethyl, difluoromethyl, trifluoromethyl, pentafluoroethyl, heptafluoropropyl, difluorochloromethyl, dichlorofluoromethyl, difluoroethyl, difluoropropyl and the like.

As used herein, the term “haloalkoxy” refers to radicals wherein one or more of the hydrogen atoms of the alkoxy group are substituted with one or more halogens. Representative examples of “haloalkoxy” groups include, but not limited to, difluoromethoxy (—OCHF₂), trifluoromethoxy (—OCF₃) or trifluoroethoxy (—OCH₂CF₃).

As used herein, the terms “amine” and “amino” refer to both unsubstituted and substituted amines and salts thereof, e.g., a moiety that can be represented by:

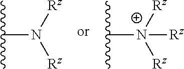

wherein R^{z} each independently represents hydrogen or a hydrocarbyl group, or R^{z} groups are taken together with the N atom to which they are attached complete a heterocyclyl having from 4 to 8 atoms in the ring structure.

As used herein, the terms “amide” and “amido” refers to a group represented by

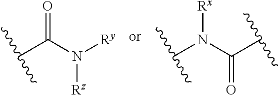

wherein R^{x}, R^{y}, and R^{z} each independently represents hydrogen or a hydrocarbyl group, or R^{y} and R^{z} are taken together with the N atom to which they are attached complete a heterocyclyl having from 4 to 8 atoms in the ring structure.

As used herein, the term “aminoalkyl” refers to an alkyl group substituted with an amino group.

As used herein, the term “hydroxyalkyl” refers to an alkyl group substituted with a hydroxy group.

As used herein, the term “cycloalkyl” alone or in combination with other term(s) refers to a cyclic hydrocarbon which is completely saturated. “Cycloalkyl” includes monocyclic, bicyclic, and tricyclic rings. Typically, a monocyclic cycloalkyl group has from 3 to about 10 carbon atoms, more typically 3 to 8 carbon atoms (e.g., C₃-C₁₀ cycloalkyl or e.g., C₃-C₆ cycloalkyl unless otherwise defined. Examples of monocyclic cycloalkyls include cyclopropyl, cyclobutyl, cyclopentyl, cyclohexyl, cycloheptyl, and the like. The second ring of a bicyclic cycloalkyl or, the second or third rings of a tricyclic cycloalkyl, may be selected from saturated, unsaturated, and aromatic rings. Cycloalkyl includes bicyclic and tricyclic molecules in which one, two or three or more atoms are shared between the two rings. The term “fused cycloalkyl” refers to a bicyclic or tricyclic cycloalkyl in which each of the rings shares two adjacent atoms with the other ring. The second ring of a fused bicyclic cycloalkyl or, the second or third rings of a fused tricyclic cycloalkyl, may be selected from saturated, unsaturated, and aromatic rings. A “cycloalkenyl” group is a cyclic hydrocarbon containing one or more double bonds. Cycloalkyls can be further substituted with alkyls, alkenyls, alkoxys, alkylthiols, aminoalkyls, carbonyl-substituted alkyls, —CF₃, —CN, and the like. A cycloalkyl may alternatively be polycyclic with more than two rings. Examples of polycyclic cycloalkyls include bridged, fused, and spirocyclic carbocyclyls. The term “cycloalkyl” as used herein may be optionally substituted, as defined above.

As used herein, the term “aryl” includes substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings (fused rings) wherein at least one of the rings is aromatic. e.g., the other cyclic rings can be cycloalkyls, cycloalkenyls, cycloalkynyls, aryls, heteroaryls, and/or heterocyclyls. The term “fused” means that the second ring is attached or formed by having two adjacent atoms in common with the first ring. The term “fused” is equivalent to the term “condensed”. Examples of aryl groups include but are not limited to phenyl, naphthyl, phenanthryl, phenol, aniline, indanyl, dihydrobenzofuranyl, dihydroisobenzofuranyl, indazoyl, imidazoyl, indolinyl, isoindolinyl, and the like. The term “aryl” as used herein may be optionally substituted, as defined above.

As used herein, the terms “heterocyclyl”, “heterocycloalkyl”, “heterocycle”, and “heterocyclic” refer to a non-aromatic, saturated or partially saturated, including monocyclic, polycyclic (e.g., bicyclic, tricyclic) bridged, or fused, ring system of 3 to 15 member having at least one heteroatom or heterogroup selected from O, N, S, S(O), S(O)₂, NH or C(O) with the remaining ring atoms being independently selected from the group consisting of carbon, oxygen, nitrogen, and sulfur. Examples of “heterocycloalkyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, indolinylmethyl, 2-azabicyclo[2.2.2]octanyl, azocinyl, chromanyl, xanthenyl and N-oxides thereof. Attachment of a heterocycloalkyl substituent can occur via either a carbon atom or a heteroatom. A heterocycloalkyl group can be optionally substituted with one or more suitable groups by one or more aforesaid groups. Preferably “heterocycloalkyl” refers to 5- to 6-membered ring selected from the group consisting of azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl, thiomorpholinyl, 1,4-dioxanyl and N-oxides thereof. The term “heterocyclyl” as used herein may be optionally substituted, as defined above.

As used herein, the term “heteroaryl” refers to substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also refers to substituted or unsubstituted aromatic or partly aromatic ring systems containing at least one heteroatom and having two or more cyclic rings (bicyclic, tricyclic, or polycyclic), containing 8 to 20 ring atoms, suitably 5 to 10 ring atoms, which may be linked covalently, or fused in which two or more atoms are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. The rings may contain an N or S atom, wherein the N or S atom is optionally oxidized, or the N atom is optionally quaternized. All heteroaryls are optionally substituted. Any suitable ring position of the heteroaryl moiety may be covalently linked to a defined chemical structure. Examples of heteroaryl include, but are not limited to: furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, thiadiazolyl, triazolyl, pyridyl, pyrimidinyl, pyrazinyl, pyridazinyl, triazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, alpha-carboline, indolizinyl, benzoisothiazolyl, benzoxazolyl, pyrrolopyridyl, furopyridinyl, purinyl, benzothiadiazolyl, benzoxadiazolyl, benzotriazolyl, benzothiadiazolyl, carbazolyl, dibenzothienyl, acridinyl and the like. The term “heteroaryl” as used herein may be optionally substituted, as defined above.

As used herein, the term “cyano” refers to —CN group.

As used herein, the term “hydroxy” or “hydroxyl” refers to —OH group.

As used herein, the term “halo” or “halogen” alone or in combination with other term(s) means chloro, fluoro, bromo, and iodo.

As used herein, the term “heteroatom” refers to an atom of any element other than carbon or hydrogen. Exemplary heteroatoms are nitrogen (N), oxygen (O), sulfur (S), and silicon (Si).

As used herein, the term “hydrocarbyl” refers to a group that is bonded through a carbon atom that does not have a ═O or ═S substituent, and typically has at least one carbon-hydrogen bond and a primarily carbon backbone but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

As used herein, the terms “polycyclyl”, “polycycle”, and “polycyclic” refer to two or more rings (e.g., cycloalkyls, cycloalkenyls, cycloalkynyls, aryls, heteroaryls, and/or heterocyclyls) in which one or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The compounds of the disclosure may contain one or more chiral centers and, therefore, exist as stereoisomers. The term “stereoisomers” when used herein consist of all enantiomers or diastereomers. These compounds may be designated by the symbols “(+)”, “(−)”, “R”, or “S”, depending on the configuration of substituents around the stereogenic carbon atom, but the skilled artisan will recognize that a structure may denote a chiral center implicitly. The presently described compounds encompasses all stereoisomers of these compounds and mixtures thereof. Mixtures of enantiomers or diastereomers may be designated “(±)” in nomenclature, but the skilled artisan will recognize that a structure may denote a chiral center implicitly.

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns, or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations and may involve the use of chiral auxiliaries. For examples, see Carreira and Kvaerno, Classics in Stereoselective Synthesis, Wiley-VCH: Weinheim, 2009.

The disclosure also embraces isotopically labeled compounds which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine, and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium, forming a deuterated compound.

As used herein, “deuterated” mean that at least one hydrogen atom is replaced by deuterium. In any sample of a deuterated compound, some discrete molecules of the compound will likely have hydrogen, rather than deuterium, at the specified position. However, the percent of molecules of the deuterated compound which have deuterium at the specified position will be much greater than would naturally occur. The deuterium at the deuterated position is enriched.

“Pharmaceutically or pharmacologically acceptable” include molecular entities and compositions that do not produce an adverse, allergic, or other untoward reaction when administered to an animal, or a human, as appropriate. For human administration, preparations should meet sterility, pyrogenicity, and general safety and purity standards as required by FDA Office of Biologics standards.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The term “pharmaceutical composition” as used herein refers to a composition comprising at least one compound as disclosed herein formulated together with one or more pharmaceutically acceptable carriers.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

“Disease,” “disorder,” and “condition” are used interchangeably herein.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like), and laboratory animals (e.g., rats, mice, guinea pigs, and the like).

A “combination therapy” is a treatment that includes the administration of two or more therapeutic agents, e.g., a compound of the disclosure and one or more additional agents, such as a RTK pathway inhibitor, a MAPKAP pathway inhibitor, a PI3K inhibitor, an mTOR inhibitor, an immunomodulatory agent, or a chemotherapeutic agent, or a combination thereof, to a patient in need thereof.

A “RTK pathway inhibitor” is an inhibitor of the RTK signaling pathway. Inhibitors of this pathway include KIT inhibitors (e.g., ripretinib, avaprinib, sunitinib, AZD3229, THE-630, and imatinib, and pharmaceutically acceptable salts thereof), EGFR inhibitors (e.g., cetuximab, osimertinib, and afatinib, and pharmaceutically acceptable salts thereof), PDGFRα inhibitors (e.g., ripretinib, JNJ10198409, or a pharmaceutically acceptable salt thereof), VEGFR inhibitors (e.g., regorafenib, axitinib, and pazopanib, and pharmaceutically acceptable salts thereof), anti-VEGF therapeutics (e.g., bevacizumab), BCR-Abl inhibitors (e.g., imatinib, nilotinib, dasatinib, or a pharmaceutically acceptable salt thereof), and an ALK inhibitor (e.g., lorlatinib and alectinib, and pharmaceutically acceptable salts thereof).

A “MAPKAP pathway inhibitor” is an inhibitor of the MAP kinase signaling pathway. Inhibitors of this pathway include Ras inhibitors (e.g., AMG-510, MRTX849, GDC-6036, MRTX-1133, RMC-9805, RMC-6291, and RMC-6236, and pharmaceutically acceptable salts thereof), RAF inhibitors (e.g., LY3009120, LXH254, RAF709, dabrafenib, vemurafenib, belvarafenib, KIN-2787, and VS-6766, and pharmaceutically acceptable salts thereof), MEK inhibitors (e.g., trametinib, selumetinib, cobimetinib, binimetinib, mirdametinib, and VS-676 and pharmaceutically acceptable salts thereof), and ERK inhibitors (e.g., ulixertinib, SCH772984, LY3214996, ravoxertinib, VX-11e, ERAS-007, and ASTX-029, and pharmaceutically acceptable salts thereof). The terms “MAPKAP pathway inhibitor” and “MAPKAP kinase inhibitor” are used interchangeably herein.

In the present specification, the term “therapeutically effective amount” means the amount of the subject compound that will elicit the biological or medical response of a tissue, system, or animal, (e.g., mammal or human) that is being sought by the researcher, veterinarian, medical doctor, or other clinician. The compounds described herein are administered in therapeutically effective amounts to treat a disorder.

“Treating” includes any effect, e.g., lessening, reducing, modulating, or eliminating, that results in the improvement of the condition, disease, disorder, and the like.

As used herein, “compounds of the disclosure”, comprise compounds of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, and Formula XVII, or a pharmaceutically acceptable salt, enantiomer, stereoisomer, or tautomer thereof.

### Compounds

In one embodiment, described herein is a compound represented by Formula I:

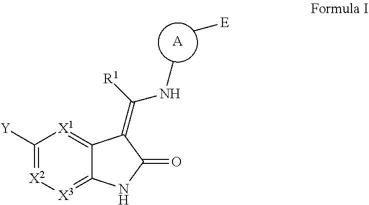

or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl; provided that not more than two of X¹, X², and X³ are N;
A is selected from the group consisting of
   optionally substituted heteroaryl wherein the optionally substituted substituent, at each occurrence, is independently selected from the group consisting of alkyl, alkoxy, halogen, haloalkyl, haloalkoxy, hydroxy, and cyano, and
   optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl;
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
Y is selected from the group consisting of

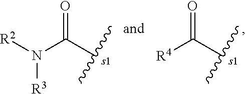

wherein s1 is the site covalently linked to the ring;
R¹ is alkyl or haloalkyl;
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano, provided that when one of R² and R³ is H, the other of R² and R³ is not H,
or when Y is

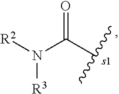

R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano; and
R⁴ is selected from the group consisting of
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano; provided that
      when X¹, X², and X³ are CH, Y is

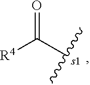

R⁴ is C₁₋₅ alkyl optionally substituted by one or more fluorine atoms, and A is an optionally substituted phenyl, E is not alkyl substituted with amine.

In some embodiments, X¹ is CH. In other embodiments, X¹ is CF or N. In some embodiments, X³ is CH. In other embodiments, X³ is CF or N. In some embodiments, X² is CH. In other embodiments, X² is CF, C-Cl, or N. In some embodiments, X¹, X², and X³ are CH.

In some embodiments, R¹ is alkyl, for example, methyl or ethyl. In some embodiments, R¹ is methyl. In some embodiments, R¹ is ethyl.

In some embodiments, A is selected from the group consisting of

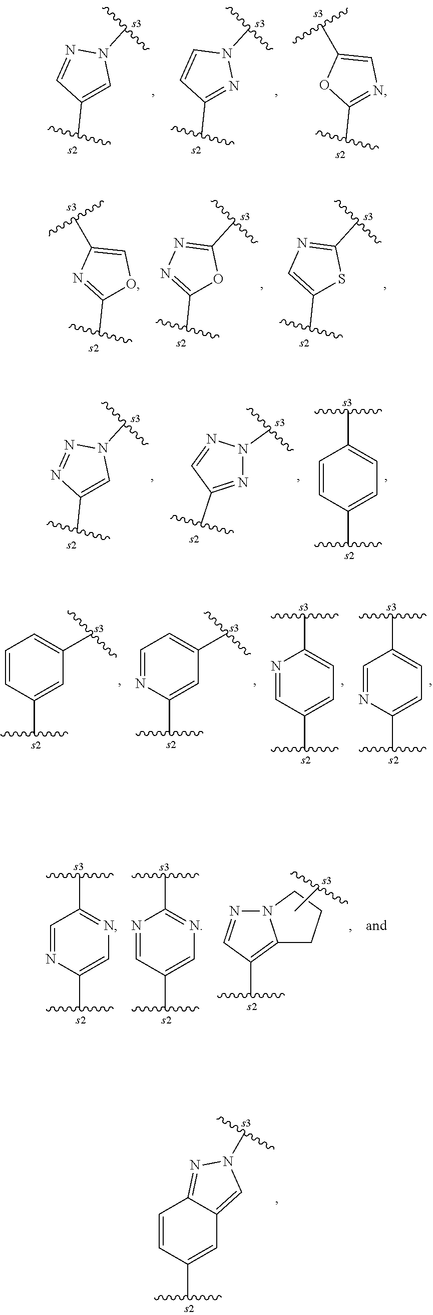

each of which is optionally substituted with alkyl, alkoxy, halogen, haloalkyl, haloalkoxy, hydroxy, or cyano, wherein s2 is the site covalently linked to —NH— and s3 is the site covalently linked to E.

In some embodiments, A is phenyl optionally substituted with halogen or alkyl.

In another embodiment, A is 5-6 membered heteroaryl optionally substituted with halogen or alkyl. In some embodiments, A is pyrazole optionally substituted with halogen or alkyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

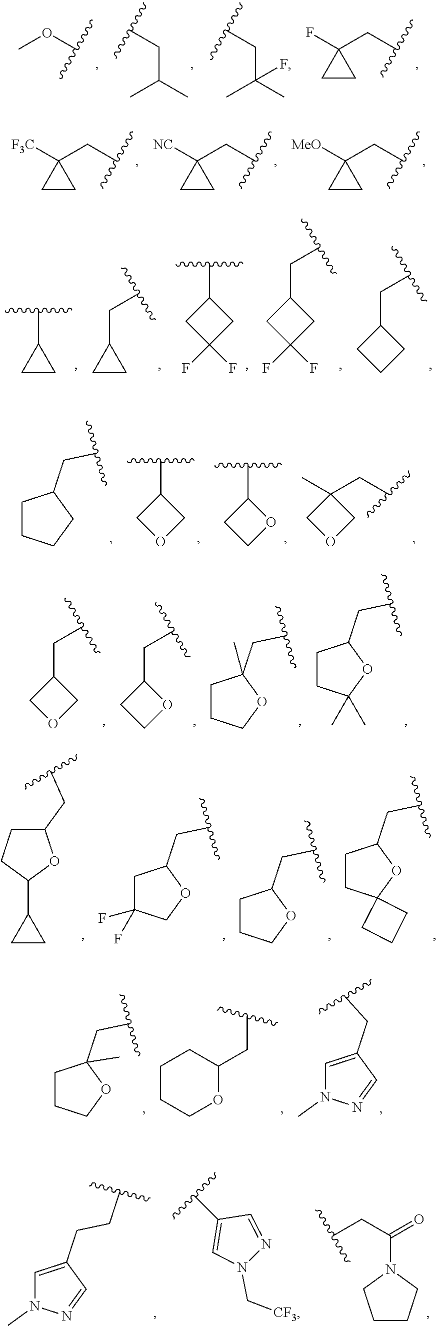

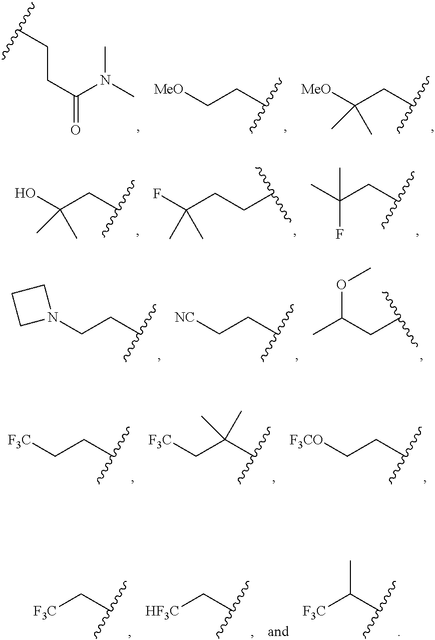

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

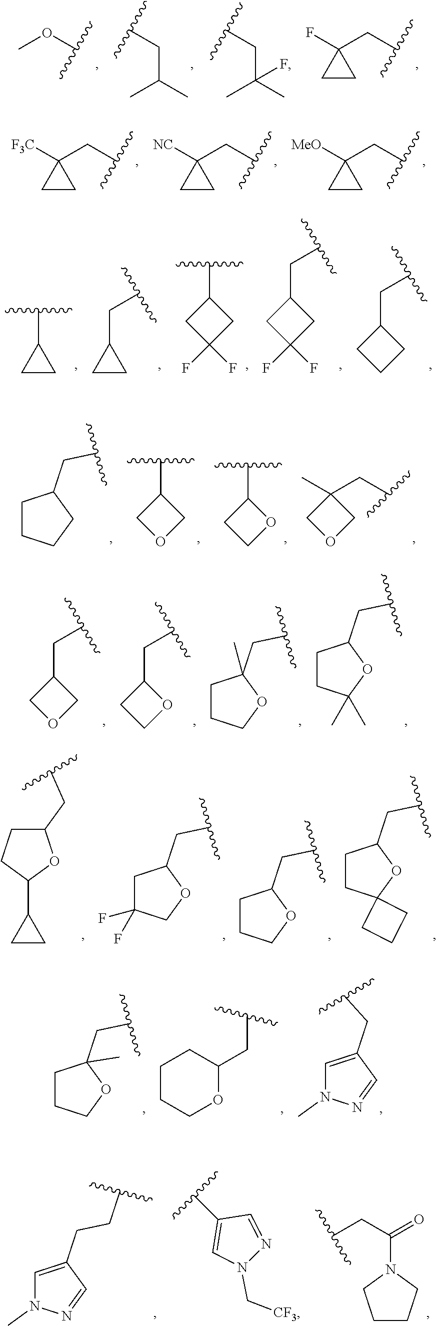

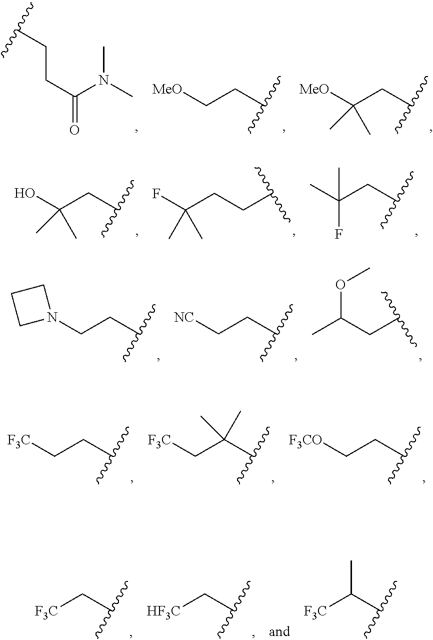

In some embodiments, R² and R³ together with the nitrogen atom to which they are attached form an optionally substituted heterocyclyl selected from the group consisting of

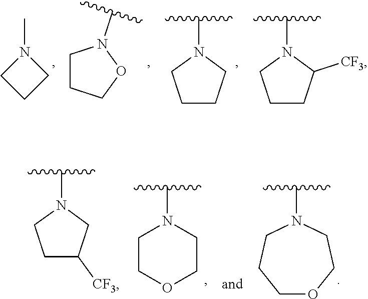

In some embodiments, R² and R³ together with the nitrogen atom to which they are attached form an optionally substituted heterocyclyl selected from the group consisting of

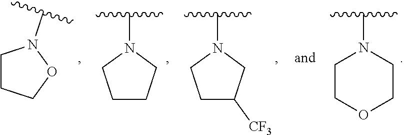

In some embodiments, R⁴ is selected from the group consisting of optionally substituted alkyl and cycloalkyl. In some embodiments, R⁴ is selected from the group consisting of methyl,

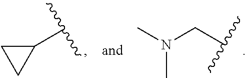

In some embodiments, E is selected from the group consisting of

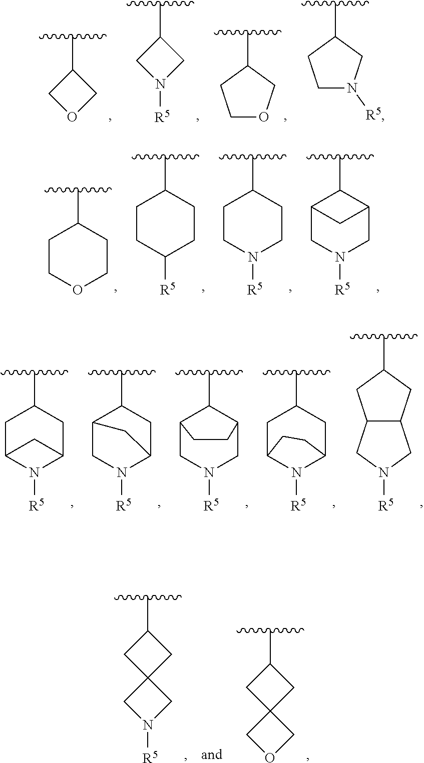

wherein R⁵ is independently H, alkyl, cycloalkyl, haloalkyl, or heterocyclyl.

In some embodiments, E is selected from the group consisting of

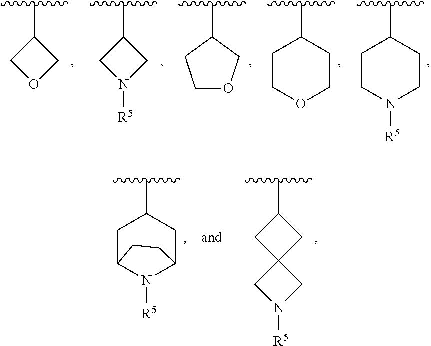

wherein R⁵ is independently H, alkyl, cycloalkyl, haloalkyl, or heterocyclyl.

In some embodiments, when A is pyrazole, E is selected from the group consisting of

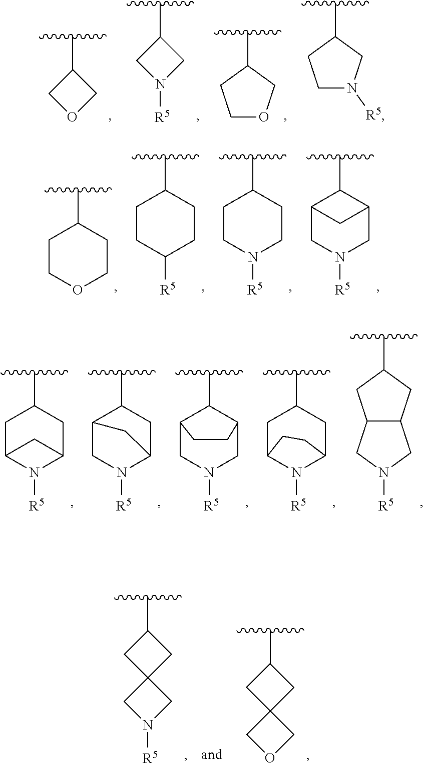

wherein R⁵ is independently H, alkyl, cycloalkyl, haloalkyl, or heterocyclyl.

In some embodiments, when A is pyrazole, E is selected from the group consisting of

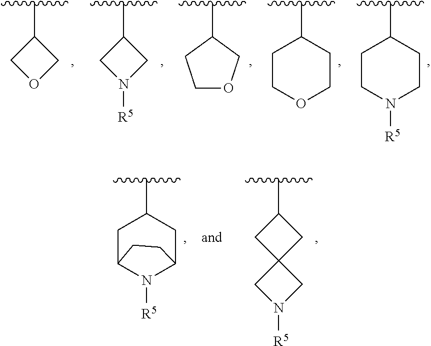

wherein R⁵ is independently H, alkyl, cycloalkyl, haloalkyl, or heterocyclyl.

In some embodiments, E is selected from the group consisting of

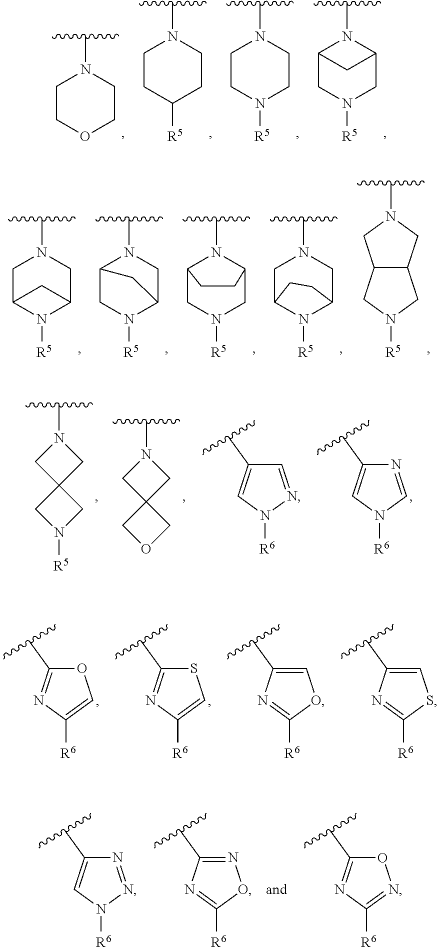

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

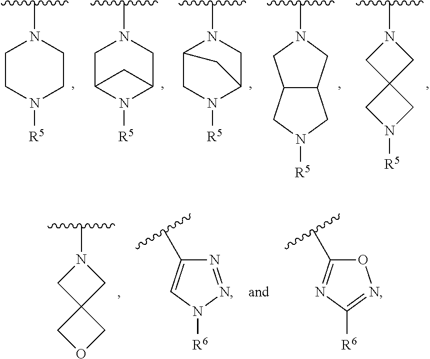

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

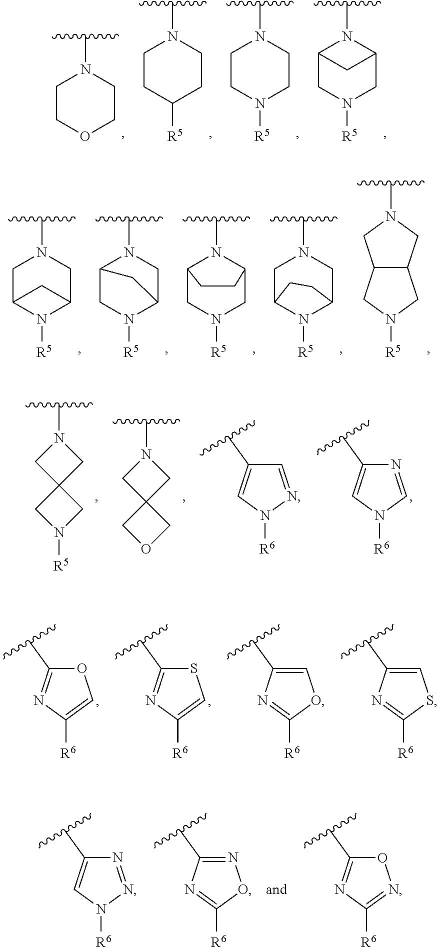

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

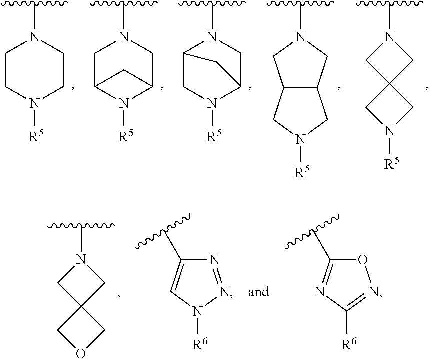

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

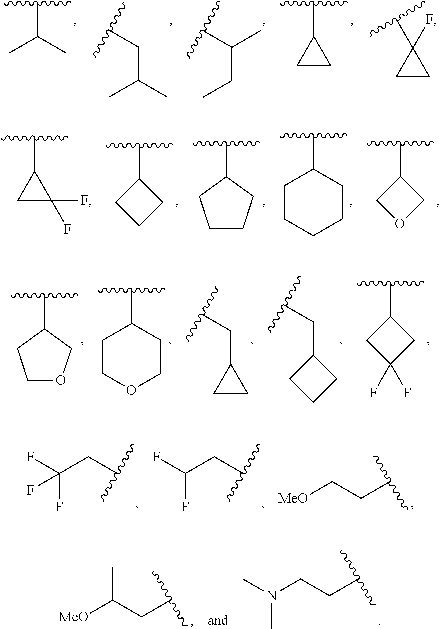

In some embodiments, E is selected from the group consisting of H, methyl, ethyl, trifluoromethyl, difluoromethyl

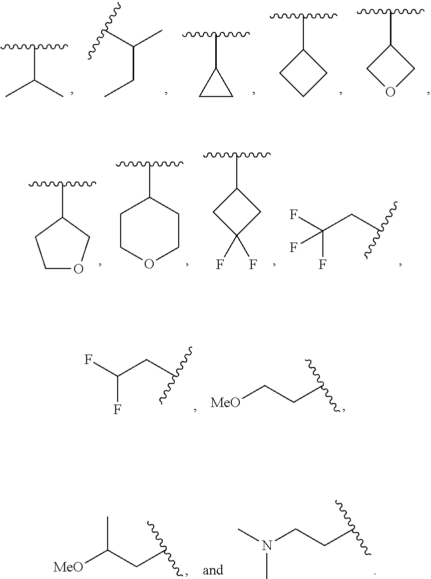

In another embodiment, described herein is a compound represented by Formula II:

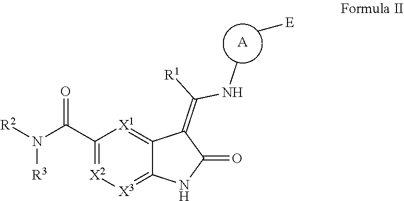

or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
   provided that not more than two of X¹, X², and X³ are N;
A is selected from the group consisting of
   optionally substituted heteroaryl wherein the optionally substituted substituent, at each occurrence, is independently selected from the group consisting of alkyl, alkoxy, halogen, haloalkyl, haloalkoxy, hydroxy, and cyano, and
   optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano, provided that when one of R² and R³ is H, the other of R² and R³ is not H, or
   R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, X¹ is CH. In other embodiments, X¹ is CF or N. In some embodiments, X³ is CH. In other embodiments, X³ is CF or N. In some embodiments, X² is CH. In other embodiments, X² is CF, C-Cl, or N. In some embodiments, X¹, X², and X³ are CH.

In some embodiments, R¹ is alkyl, for example, methyl or ethyl. In some embodiments, R¹ is methyl. In some embodiments, R¹ is ethyl.

In some embodiments, A is selected from the group consisting of

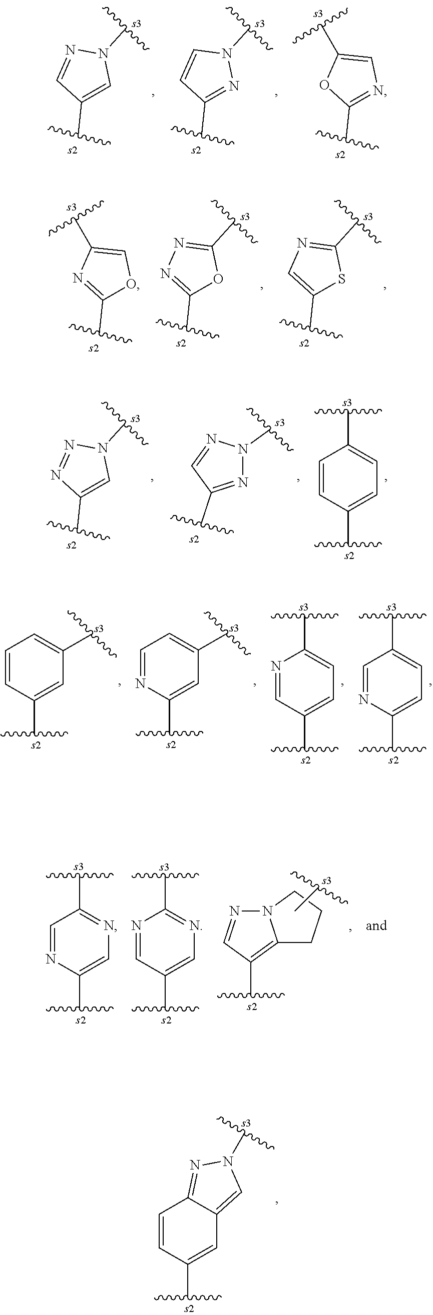

each of which is optionally substituted with alkyl, alkoxy, halogen, haloalkyl, haloalkoxy, hydroxy, or cyano, wherein s2 is the site covalently linked to —NH— and s3 is the site covalently linked to E.

In some embodiments, A is phenyl optionally substituted with halogen or alkyl.

In another embodiment, A is 5-6 membered heteroaryl optionally substituted with halogen or alkyl. In some embodiments, A is pyrazole optionally substituted with halogen or alkyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

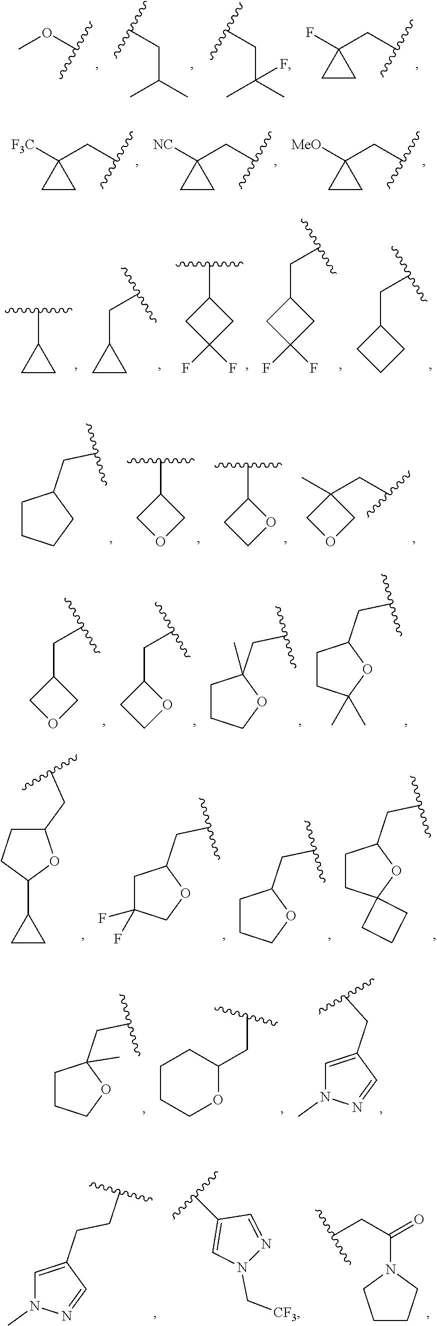

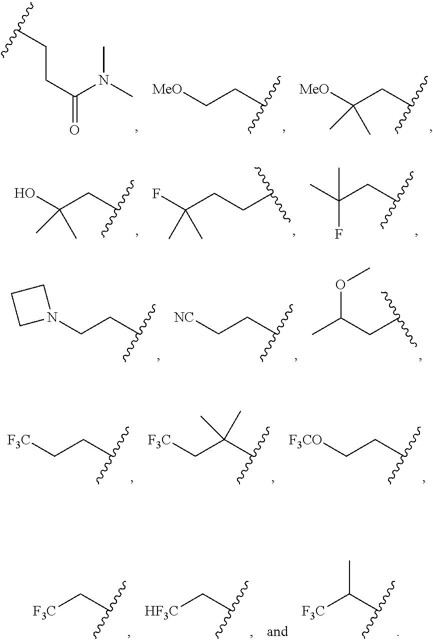

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

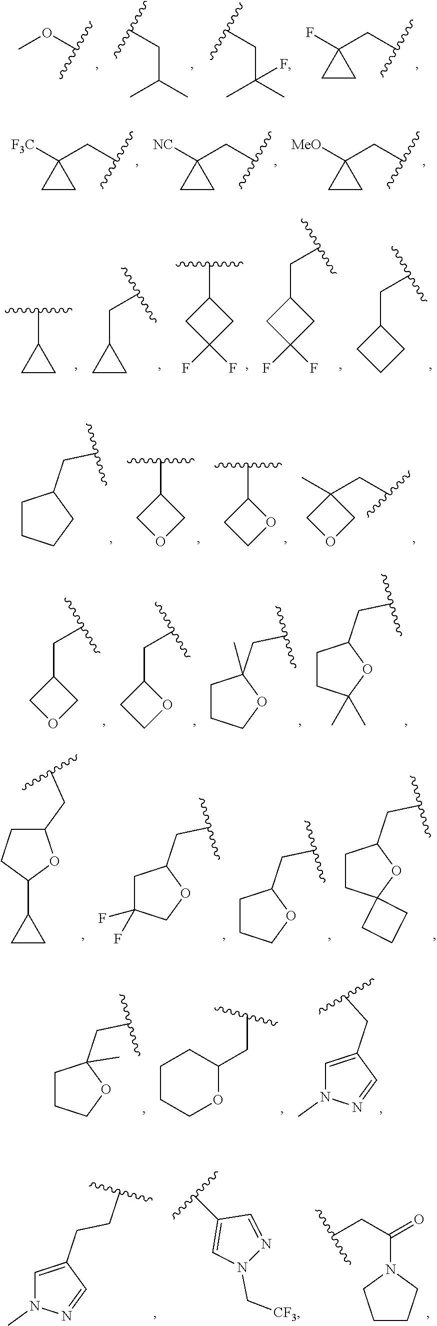

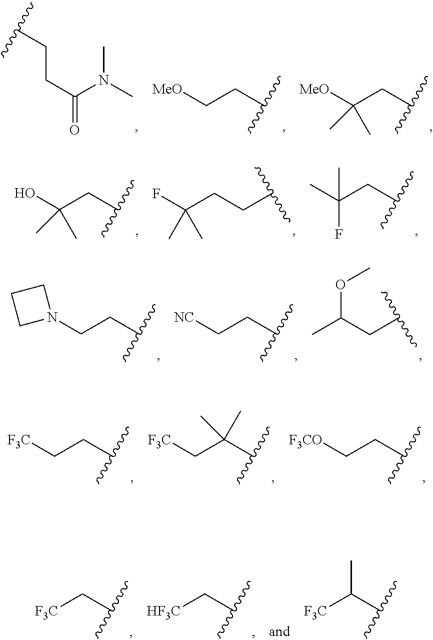

In some embodiments, R² and R³ together with the nitrogen atom to which they are attached form an optionally substituted heterocyclyl selected from the group consisting of

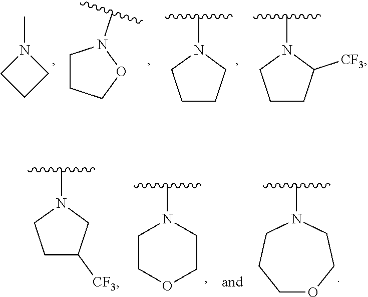

In some embodiments, R² and R³ together with the nitrogen atom to which they are attached form an optionally substituted heterocyclyl selected from the group consisting of

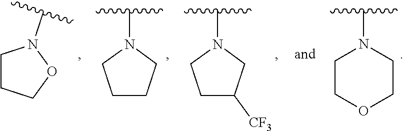

In some embodiments, E is selected from the group consisting of

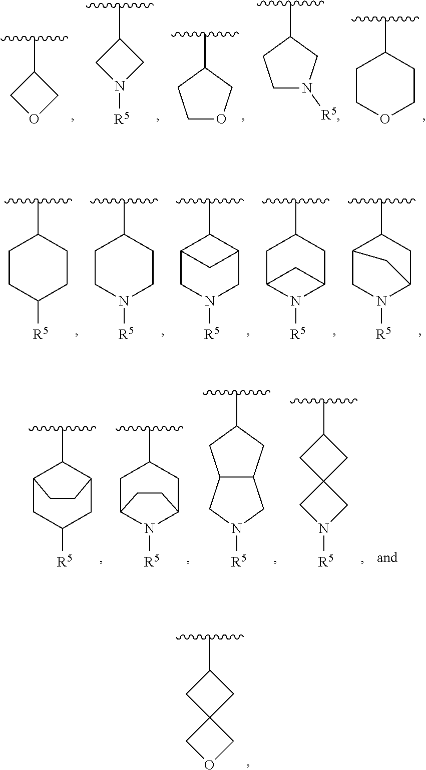

wherein R⁵ is independently H, alkyl, cycloalkyl, haloalkyl, or heterocyclyl.

In some embodiments, E is selected from the group consisting of

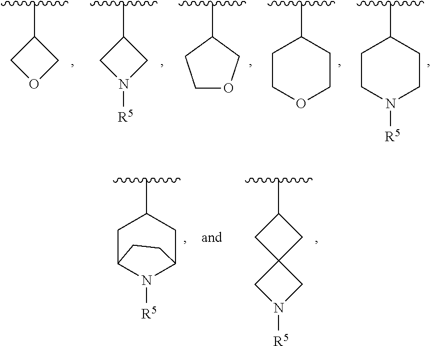

wherein R⁵ is independently H, alkyl, cycloalkyl, haloalkyl, or heterocyclyl.

In some embodiments, when A is pyrazole, E is selected from the group consisting of

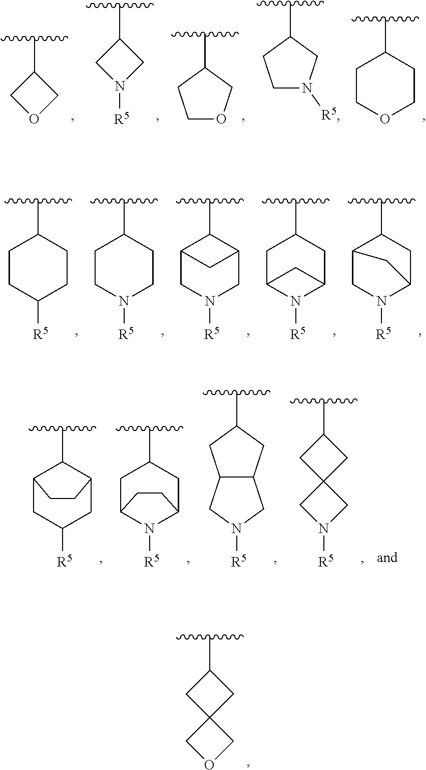

wherein R⁵ is independently H, alkyl, cycloalkyl, haloalkyl, or heterocyclyl.

In some embodiments, when A is pyrazole, E is selected from the group consisting of

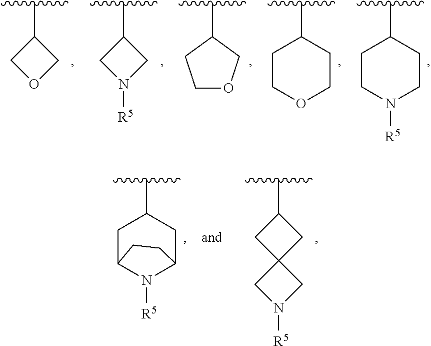

wherein R⁵ is independently H, alkyl, cycloalkyl, haloalkyl, or heterocyclyl.

In some embodiments, E is selected from the group consisting of

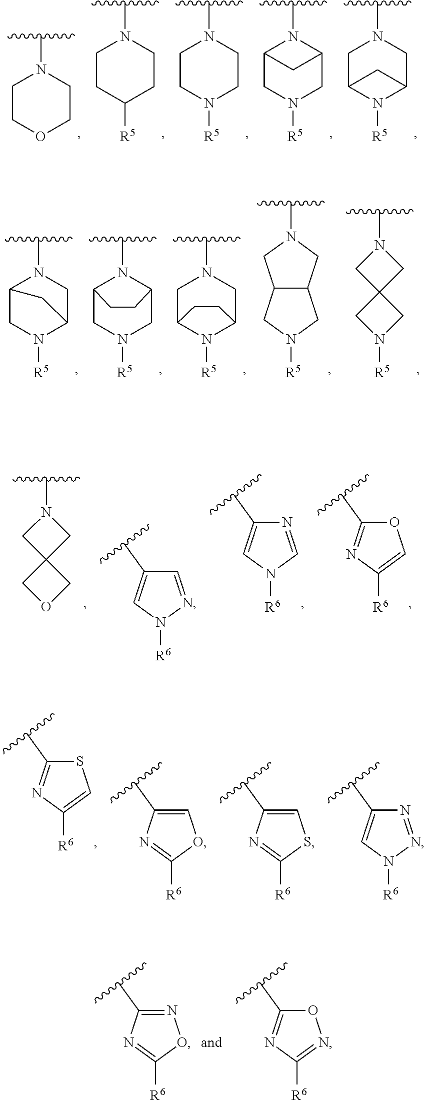

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

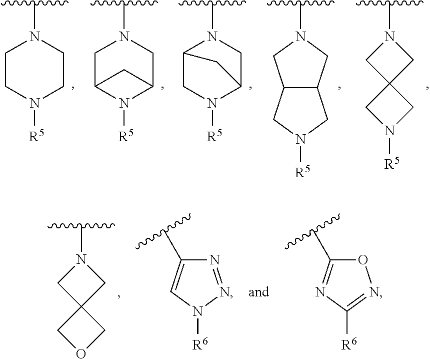

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

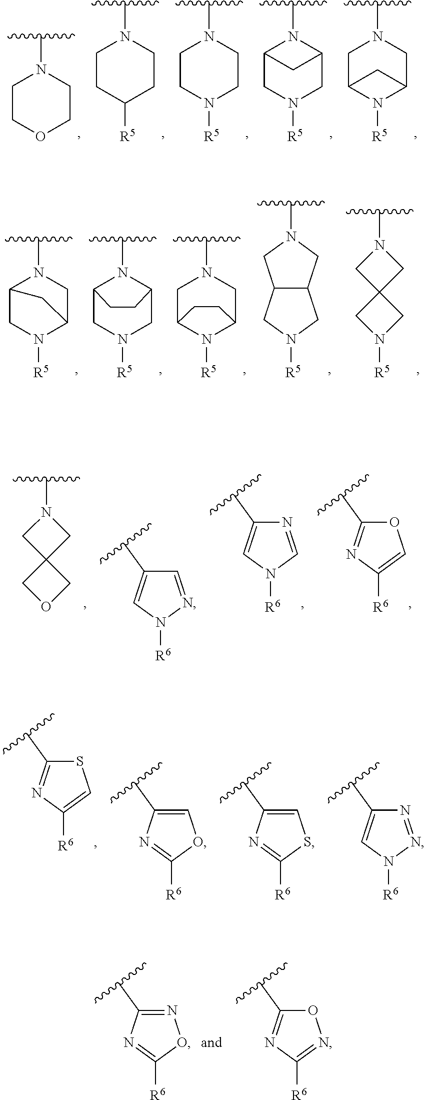

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

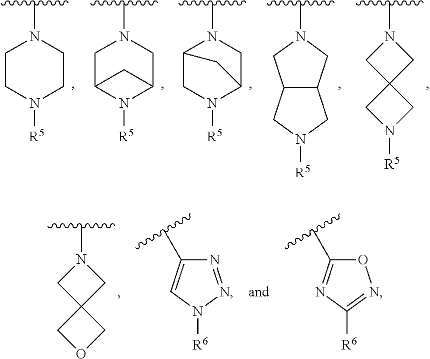

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

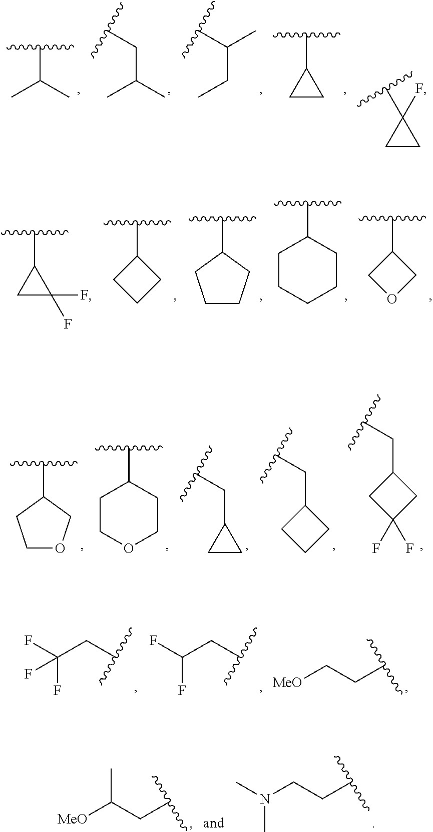

In some embodiments, E is selected from the group consisting of H, methyl, ethyl, trifluoromethyl, difluoromethyl,

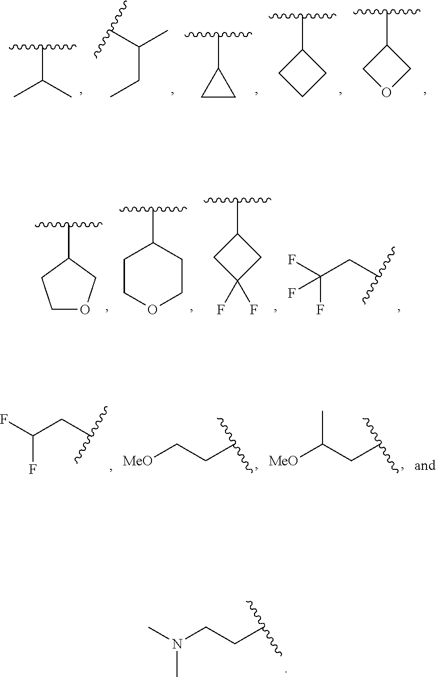

In one embodiment, described herein is a compound represented by Formula III:

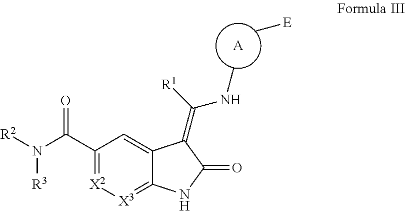

or a pharmaceutically acceptable salt, enantiomer, stereoisomer, or tautomer thereof, wherein:
X³ is selected from the group consisting of N, CH, and CF;
X² is selected from the group consisting of N, CH, CF, and C-Cl;
A is selected from the group consisting of
   optionally substituted heteroaryl wherein the optionally substituted substituent, at each occurrence, is independently selected from the group consisting of alkyl, alkoxy, halogen, haloalkyl, haloalkoxy, hydroxy, and cyano, and
   optionally substituted phenyl wherein the optionally substituted substituent, at each occurrence, is independently selected from the group consisting of alkyl, alkoxy, halogen, haloalkyl, haloalkoxy, hydroxy, and cyano; E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further
   optionally substituted with alkoxy or cycloalkyl, and optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
   R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, X³ is CH. In other embodiments, X³ is CF or N. In some embodiments, X² is CH. In other embodiments, X² is CF, C-Cl, or N.

In some embodiments, R¹ is alkyl, for example, methyl or ethyl. In some embodiments, R¹ is methyl. In some embodiments, R¹ is ethyl.

In some embodiments, A is selected from the group consisting of

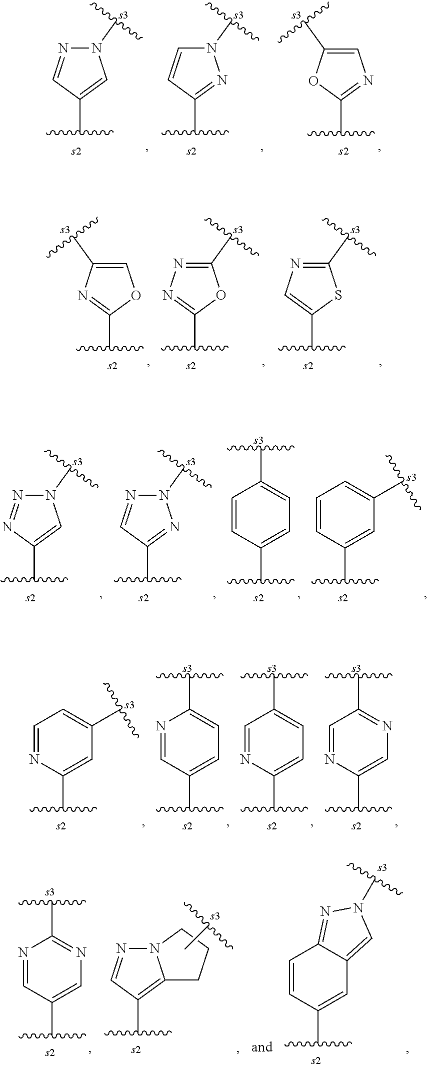

each of which is optionally substituted with alkyl, alkoxy, halogen, haloalkyl, haloalkoxy, hydroxy, or cyano, wherein s2 is the site covalently linked to —NH— and s3 is the site covalently linked to E.

In some embodiments, A is phenyl optionally substituted with halogen or alkyl.

In another embodiment, A is 5-6 membered heteroaryl optionally substituted with halogen or alkyl. In some embodiments, A is pyrazole optionally substituted with halogen or alkyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

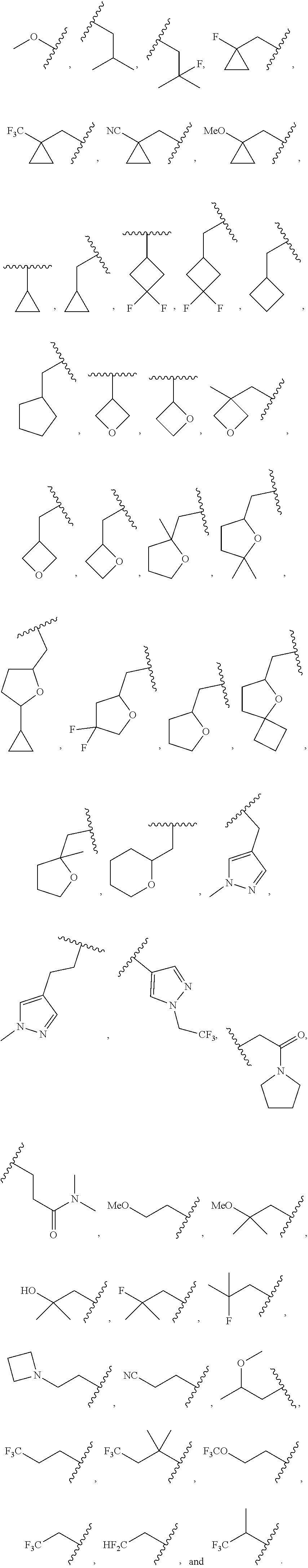

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

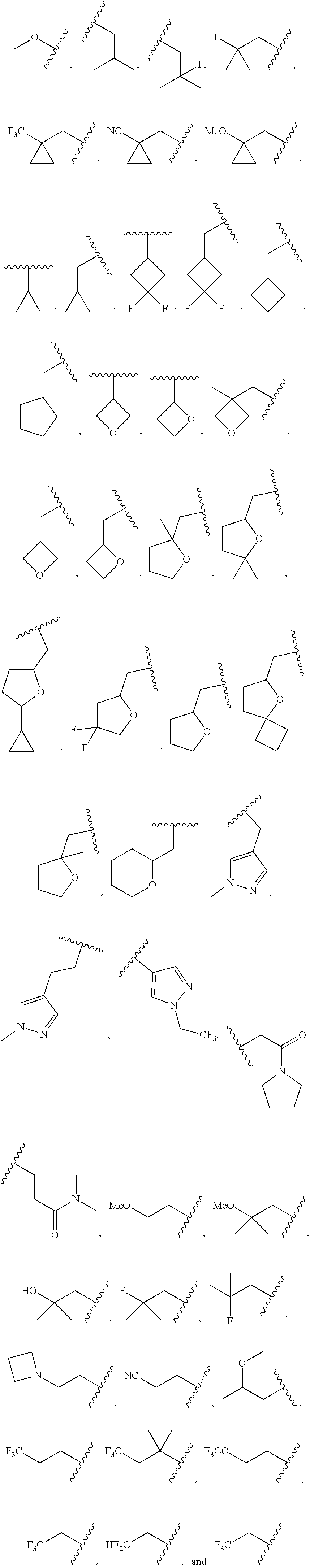

In some embodiments, R² and R³ together with the nitrogen atom to which they are attached form an optionally substituted heterocyclyl selected from the group consisting of

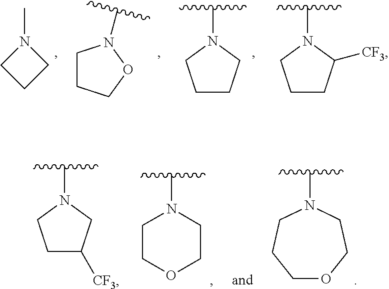

In some embodiments, R² and R³ together with the nitrogen atom to which they are attached form an optionally substituted heterocyclyl selected from the group consisting of

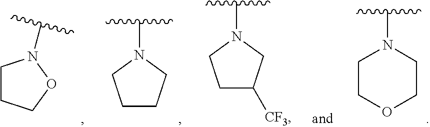 [SMILES: *N1CCCO1 ; *N1CCCC1 ; *N1CCC(C(F)(F)F)C1 ; *N1CCOCC1]

In some embodiments, E is selected from the group consisting of

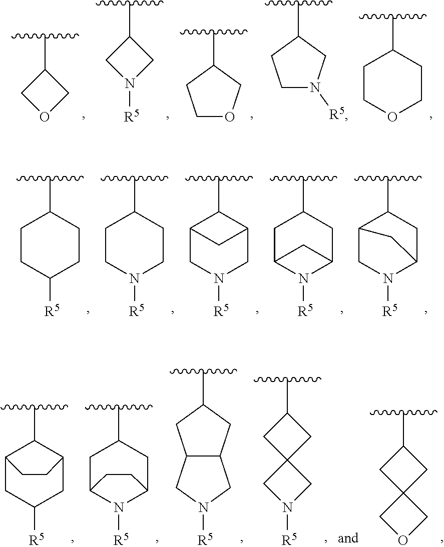

wherein R⁵ is independently H, alkyl, cycloalkyl, haloalkyl, or heterocyclyl.

In some embodiments, E is selected from the group consisting of

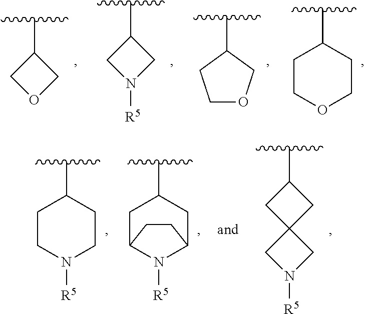

wherein R⁵ is independently H, alkyl, cycloalkyl, haloalkyl, or heterocyclyl.

In some embodiments, when A is pyrazole, E is selected from the group consisting of

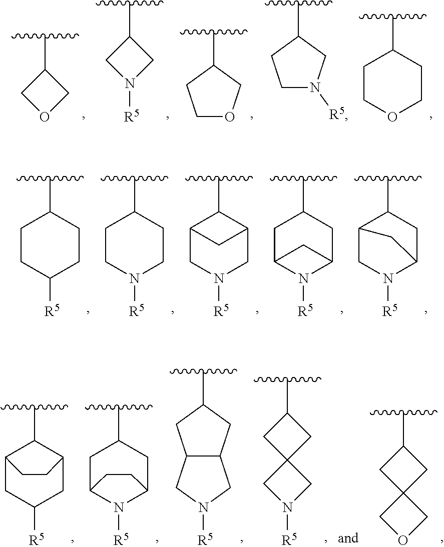

wherein R⁵ is independently H, alkyl, cycloalkyl, haloalkyl, or heterocyclyl.

In some embodiments, when A is pyrazole, E is selected from the group consisting of

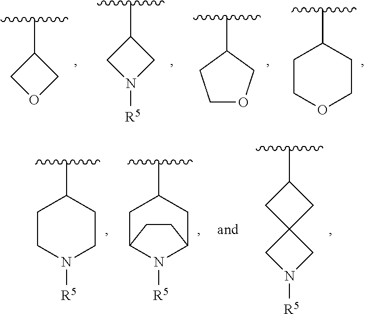

wherein R⁵ is independently H, alkyl, cycloalkyl, haloalkyl, or heterocyclyl.

In some embodiments, E is selected from the group consisting of

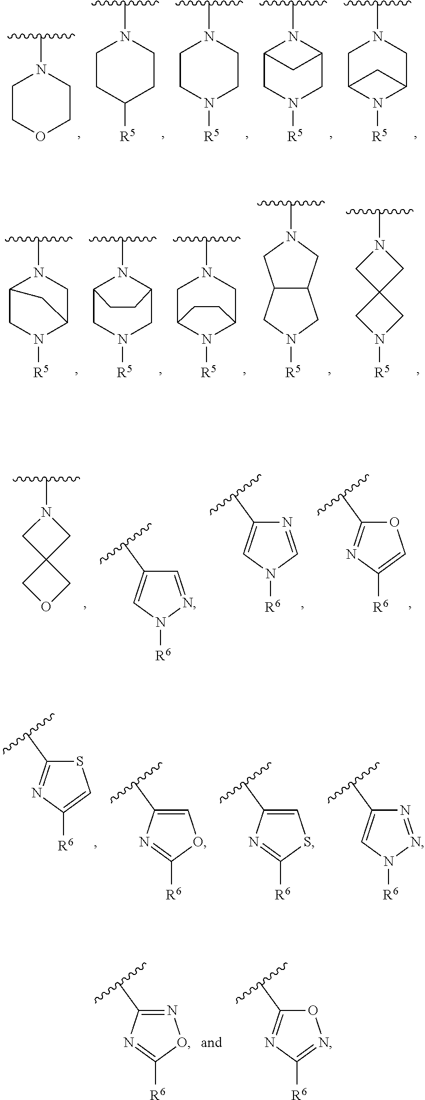

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

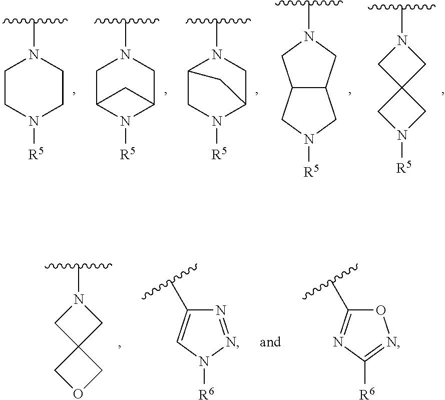

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

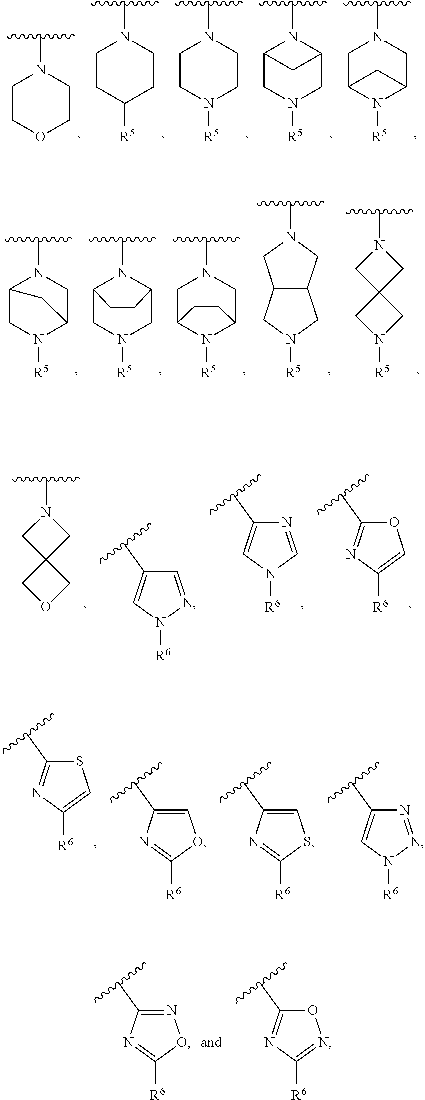

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

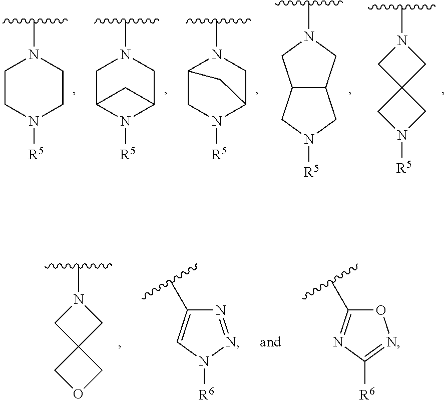

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

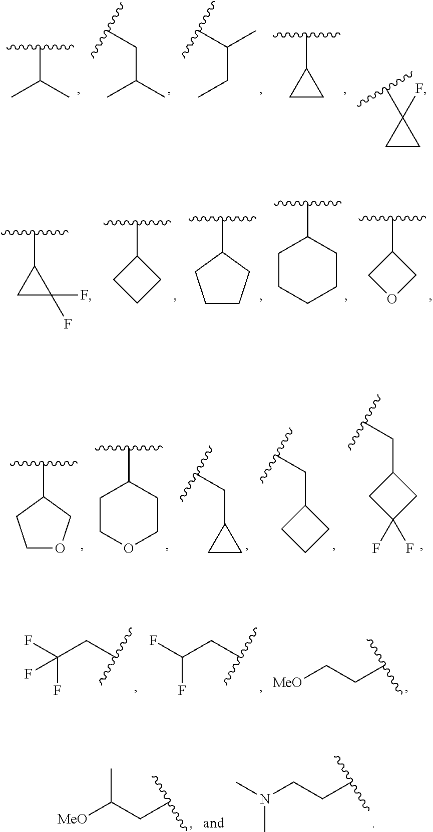

In some embodiments, E is selected from the group consisting of H, methyl, ethyl, trifluoromethyl, difluoromethyl,

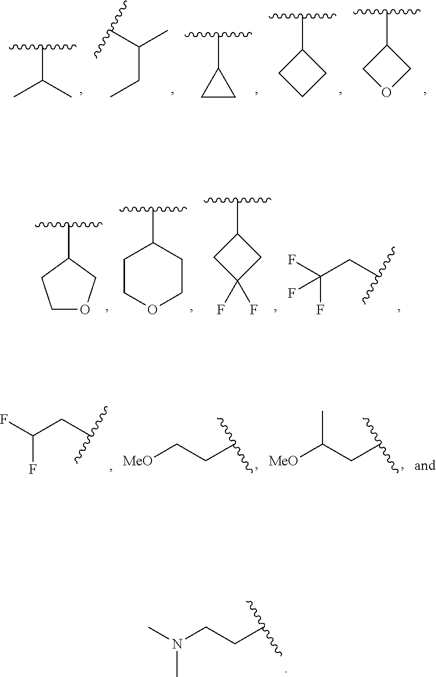

In one embodiment, described herein is a compound represented by Formula IV:

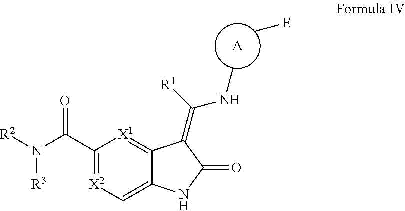

or a pharmaceutically acceptable salt, enantiomer, stereoisomer, or tautomer thereof, wherein:
X¹ is selected from the group consisting of N, CH, and CF;
X² is selected from the group consisting of N, CH, CF, and C-Cl;
A is selected from the group consisting of
   optionally substituted heteroaryl wherein the optionally substituted substituent, at each occurrence, is independently selected from the group consisting of alkyl, alkoxy, halogen, haloalkyl, haloalkoxy, hydroxy, and cyano, and
   optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, X¹ is CH. In other embodiments, X¹ is CF or NIn some embodiments, X² is CH. In other embodiments, X² is CF, C-Cl, or N.

In some embodiments, R¹ is alkyl, for example, methyl or ethyl. In some embodiments, R¹ is methyl. In some embodiments, R¹ is ethyl.

In some embodiments, A is selected from the group consisting of

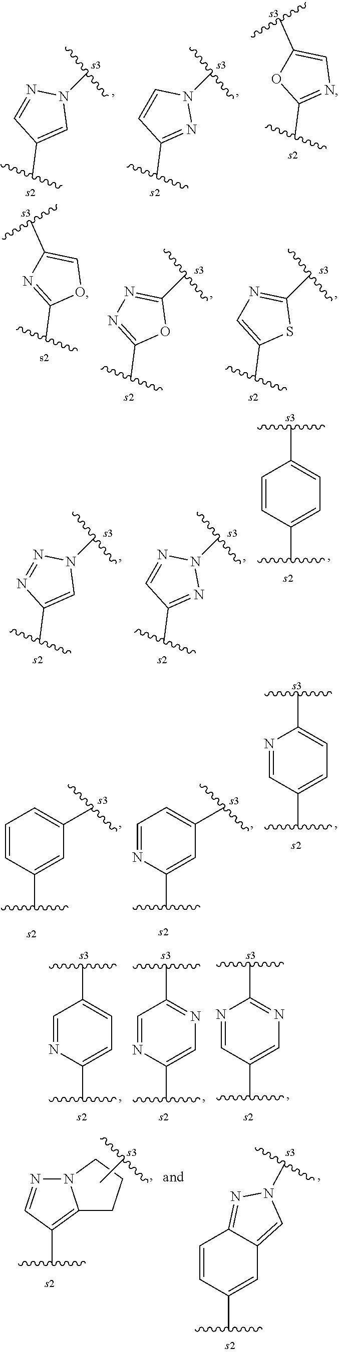

each of which is optionally substituted with alkyl, alkoxy, halogen, haloalkyl, haloalkoxy, hydroxy, or cyano, wherein s2 is the site covalently linked to —NH— and s3 is the site covalently linked to E.

In some embodiments, A is phenyl optionally substituted with halogen or alkyl.

In another embodiment, A is 5-6 membered heteroaryl optionally substituted with halogen or alkyl. In some embodiments, A is pyrazole optionally substituted with halogen or alkyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

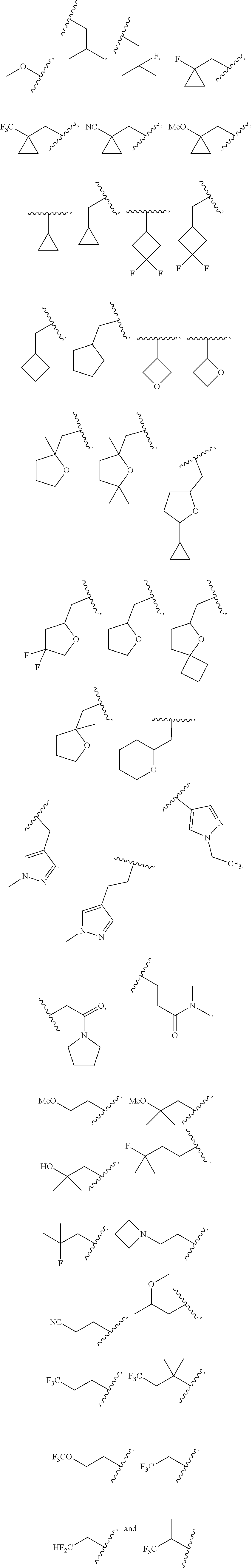

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

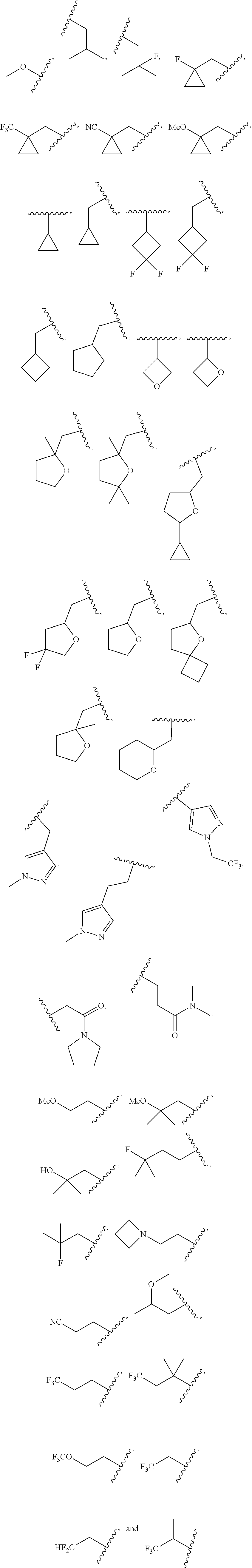

In some embodiments, R² and R³ together with the nitrogen atom to which they are attached form an optionally substituted heterocyclyl selected from the group consisting of

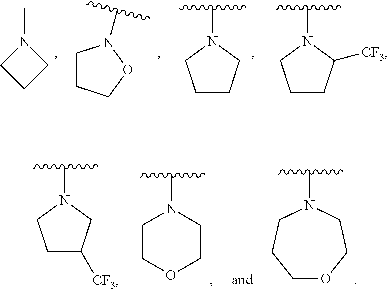

In some embodiments, R² and R³ together with the nitrogen atom to which they are attached form an optionally substituted heterocyclyl selected from the group consisting of

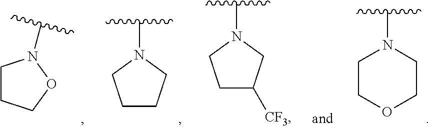 [SMILES: *N1CCCO1 ; *N1CCCC1 ; *N1CCC(C(F)(F)F)C1 ; *N1CCOCC1]

In some embodiments, E is selected from the group consisting of

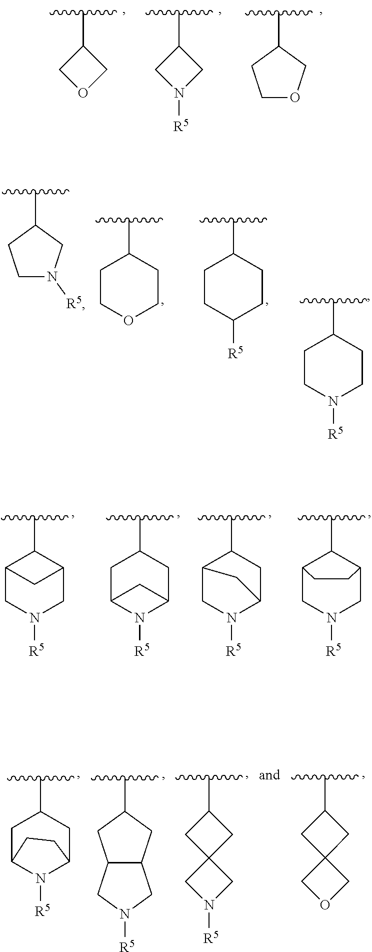

wherein R⁵ is independently H, alkyl, cycloalkyl, haloalkyl, or heterocyclyl.

In some embodiments, E is selected from the group consisting of

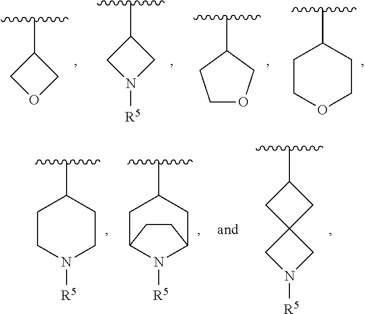

wherein R⁵ is independently H, alkyl, cycloalkyl, haloalkyl, or heterocyclyl.

In some embodiments, when A is pyrazole, E is selected from the group consisting of

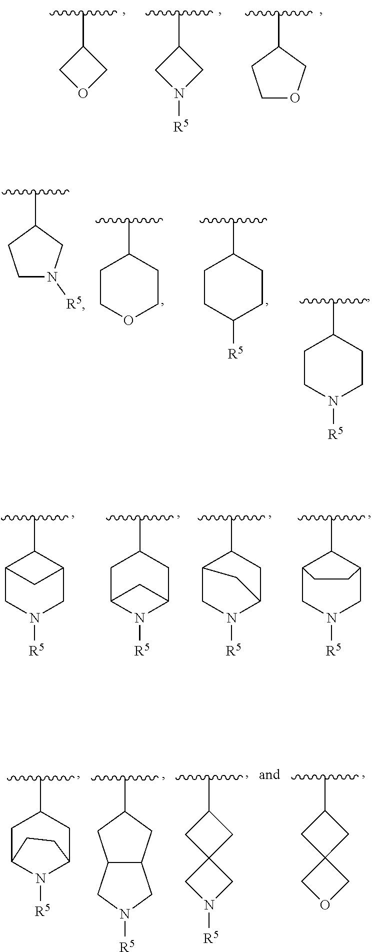

wherein R⁵ is independently H, alkyl, cycloalkyl, haloalkyl, or heterocyclyl.

In some embodiments, when A is pyrazole, E is selected from the group consisting of

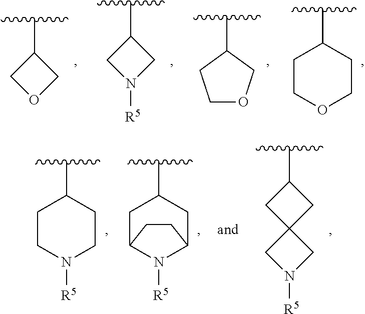

wherein R⁵ is independently H, alkyl, cycloalkyl, haloalkyl, or heterocyclyl.

In some embodiments, E is selected from the group consisting of

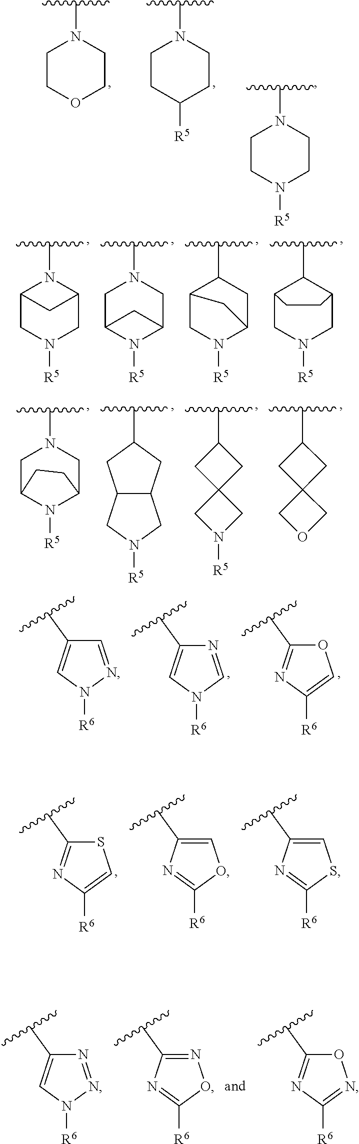

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

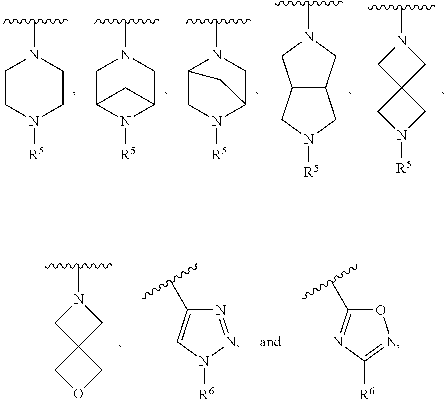

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

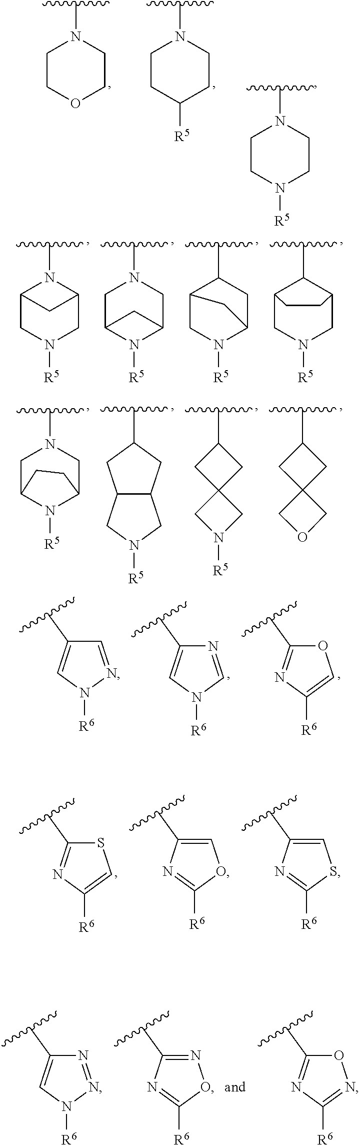

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

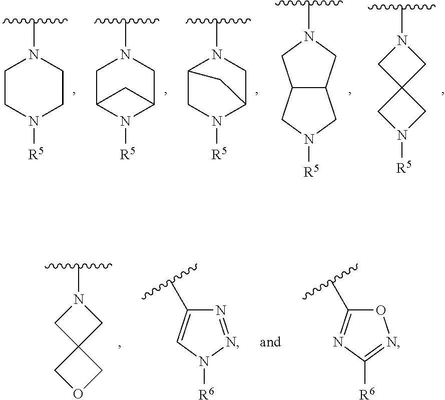

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

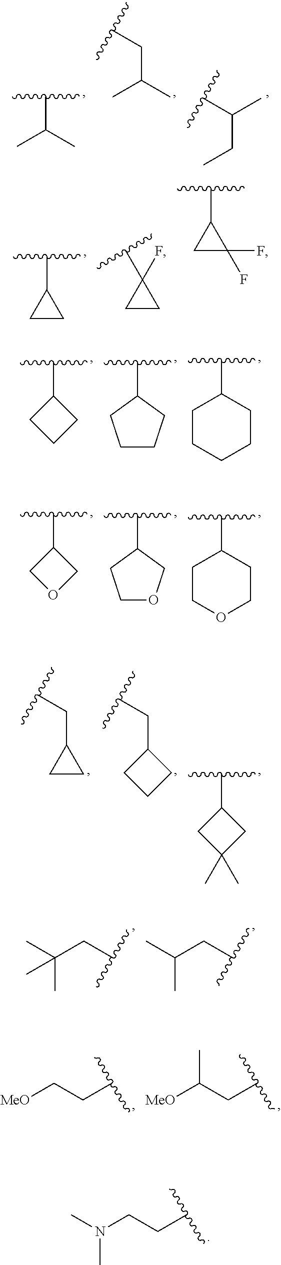

In some embodiments, E is selected from the group consisting of H, methyl, ethyl, trifluoromethyl, difluoromethyl,

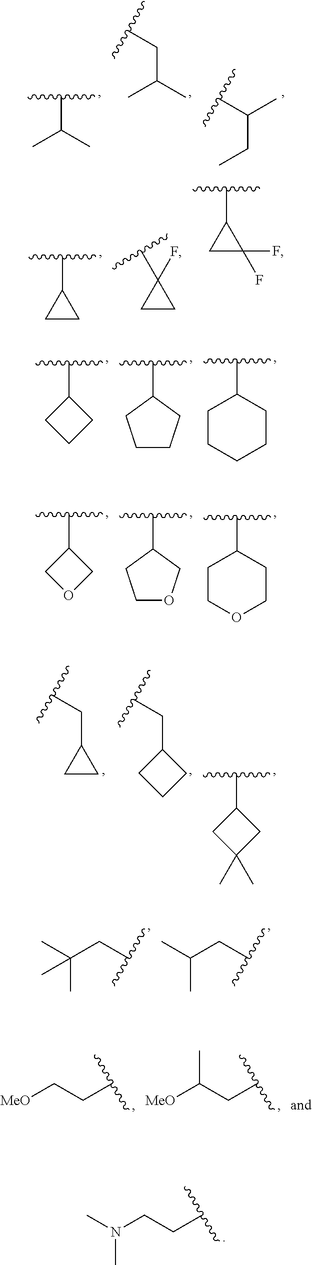

In one embodiment, described herein is a compound represented by Formula V:

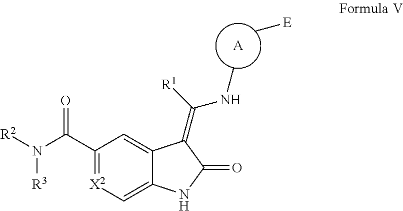

or a pharmaceutically acceptable salt, enantiomer, stereoisomer, or tautomer thereof, wherein:
X² is selected from the group consisting of N, CH, CF, and C-Cl;
A is selected from the group consisting of
   optionally substituted heteroaryl wherein the optionally substituted substituent, at each occurrence, is independently selected from the group consisting of alkyl, alkoxy, halogen, haloalkyl, haloalkoxy, hydroxy, and cyano, and
   optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, X² is CH. In other embodiments, X² is CF, C-Cl, or N.

In some embodiments, R¹ is alkyl, for example, methyl or ethyl. In some embodiments, R¹ is methyl. In some embodiments, R¹ is ethyl.

In some embodiments, A is selected from the group consisting of

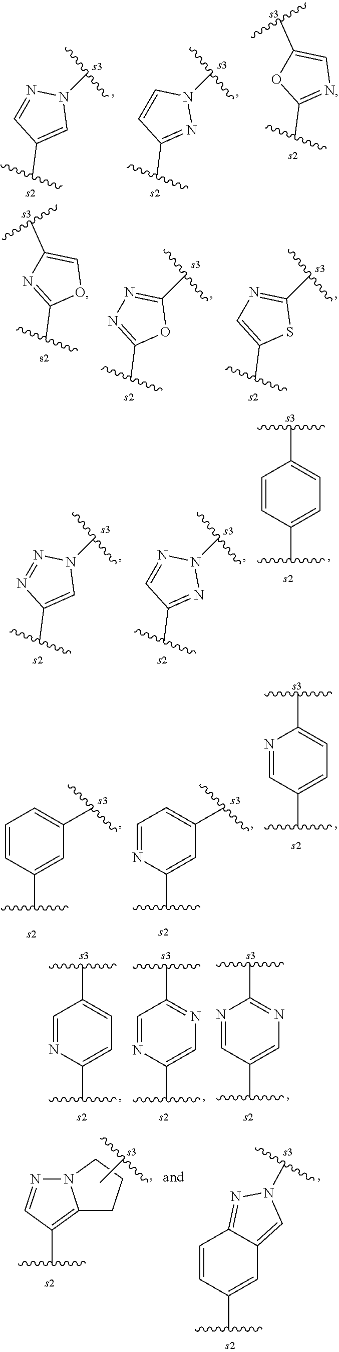

each of which is optionally substituted with alkyl, alkoxy, halogen, haloalkyl, haloalkoxy, hydroxy, or cyano, wherein s2 is the site covalently linked to —NH— and s3 is the site covalently linked to E.

In some embodiments, A is phenyl optionally substituted with halogen or alkyl.

In another embodiment, A is 5-6 membered heteroaryl optionally substituted with halogen or alkyl. In some embodiments, A is pyrazole optionally substituted with halogen or alkyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

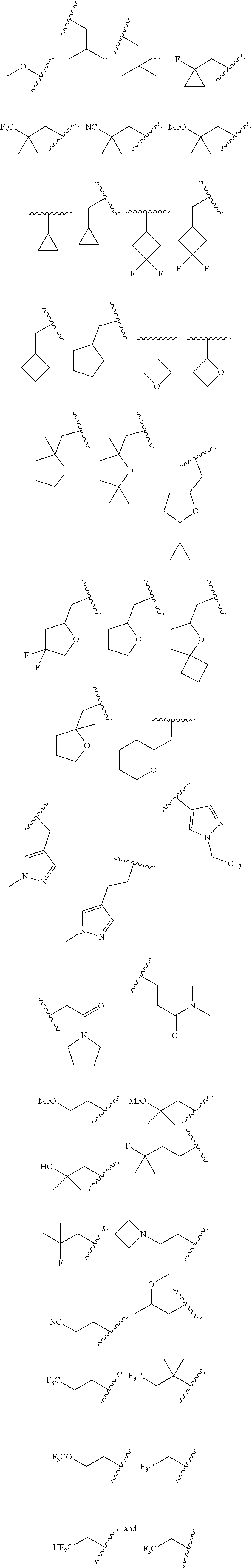

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

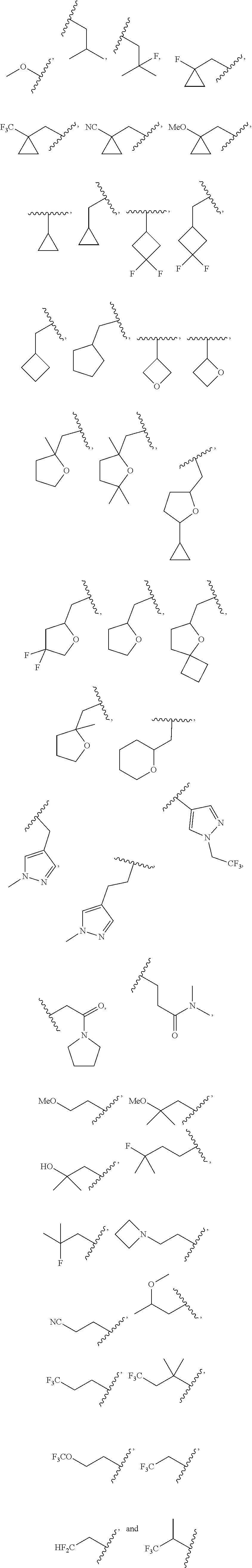

In some embodiments, R² and R³ together with the nitrogen atom to which they are attached form an optionally substituted heterocyclyl selected from the group consisting of

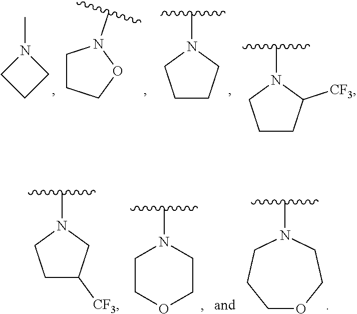

In some embodiments, R² and R³ together with the nitrogen atom to which they are attached form an optionally substituted heterocyclyl selected from the group consisting of

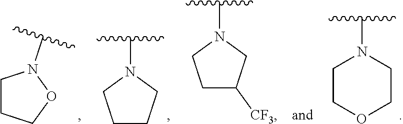

In some embodiments, E is selected from the group consisting of

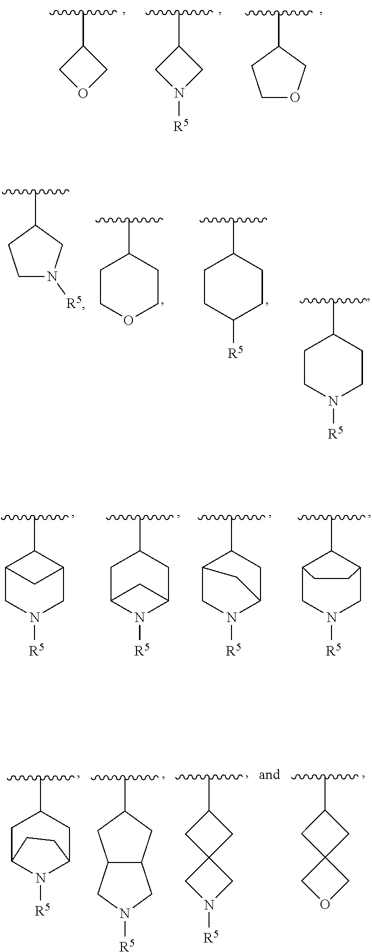

wherein R⁵ is independently H, alkyl, cycloalkyl, haloalkyl, or heterocyclyl.

In some embodiments, E is selected from the group consisting of

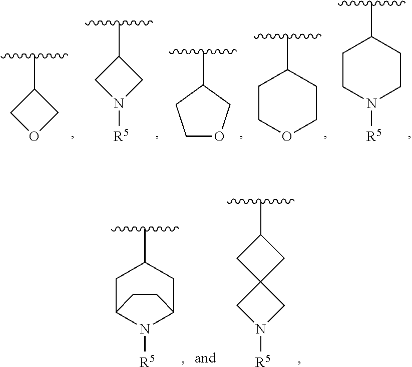

wherein R⁵ is independently H, alkyl, cycloalkyl, haloalkyl, or heterocyclyl.

In some embodiments, when A is pyrazole, E is selected from the group consisting of

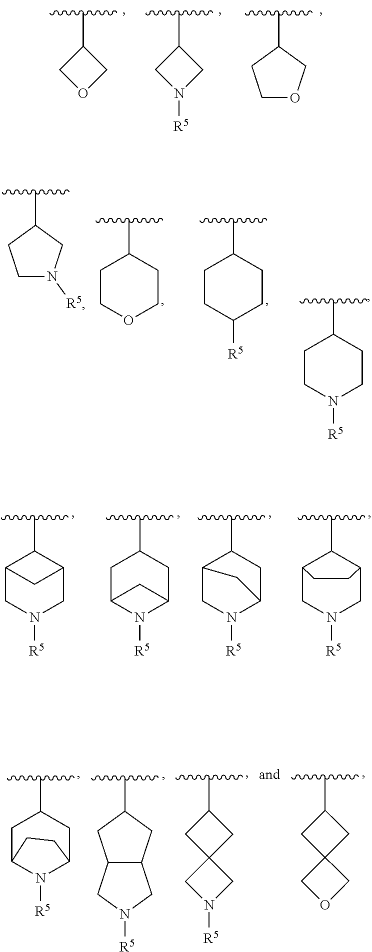

wherein R⁵ is independently H, alkyl, cycloalkyl, haloalkyl, or heterocyclyl.

In some embodiments, when A is pyrazole, E is selected from the group consisting of

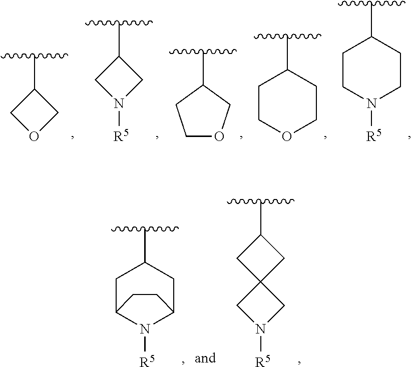

wherein R⁵ is independently H, alkyl, cycloalkyl, haloalkyl, or heterocyclyl.

In some embodiments, E is selected from the group consisting of

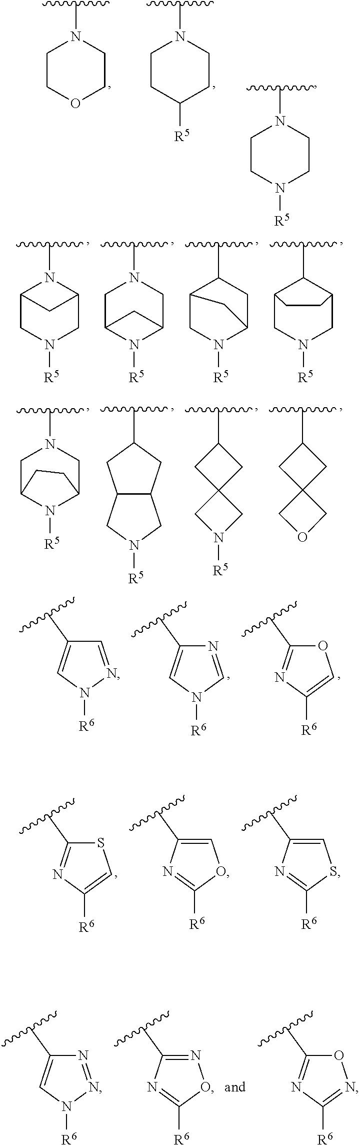

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

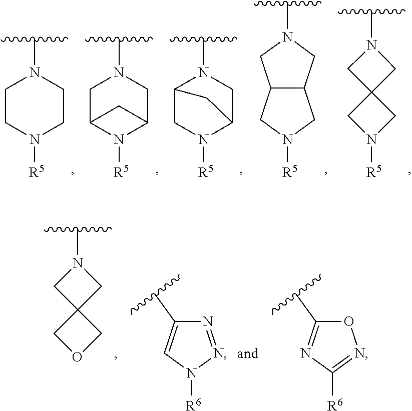

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

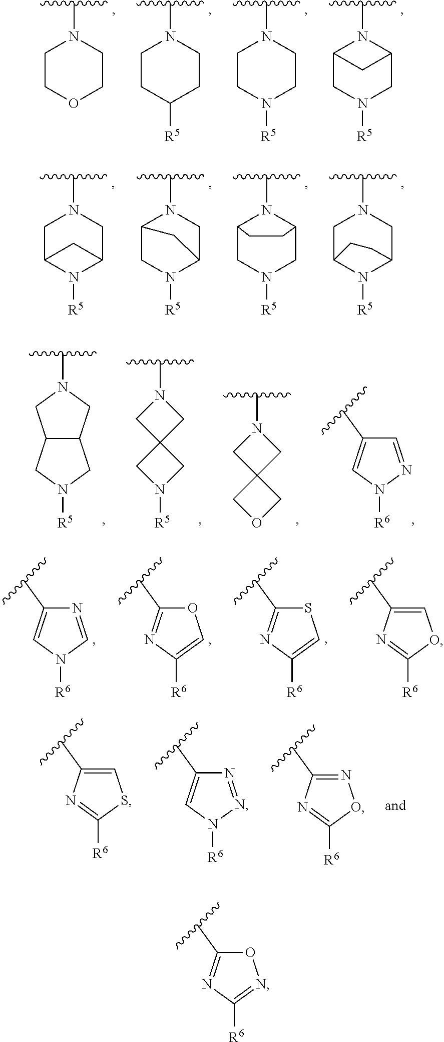

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

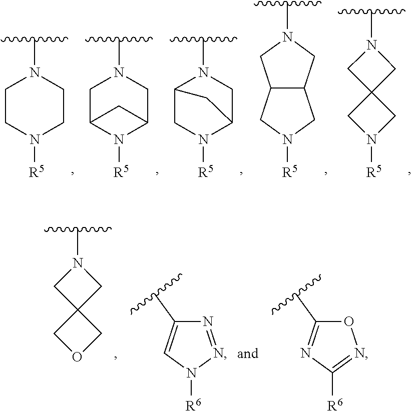

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

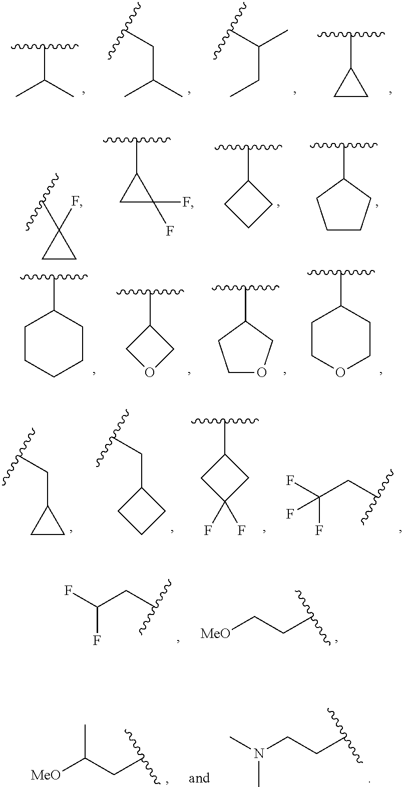

In some embodiments, E is selected from the group consisting of H, methyl, ethyl, trifluoromethyl, difluoromethyl,

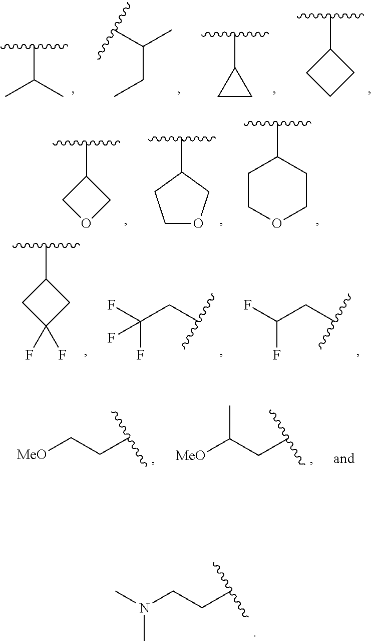

In another embodiment, described herein is a compound represented by Formula VI:

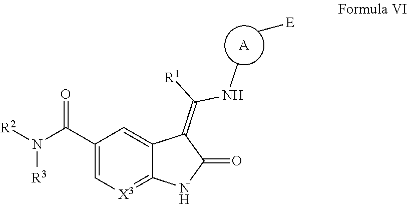

or a pharmaceutically acceptable salt, enantiomer, stereoisomer, or tautomer thereof, wherein:
X³ is selected from the group consisting of N, CH, and CF;
A is selected from the group consisting of
   optionally substituted heteroaryl wherein the optionally substituted substituent, at each occurrence, is independently selected from the group consisting of alkyl, alkoxy, halogen, haloalkyl, haloalkoxy, hydroxy, and cyano, and
   optionally substituted phenyl wherein the optionally substituted substituent, at each occurrence, is independently selected from the group consisting of alkyl, alkoxy, halogen, haloalkyl, haloalkoxy, hydroxy, and cyano; E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, X³ is CH. In other embodiments, X³ is CF or N.

In some embodiments, R¹ is alkyl, for example, methyl or ethyl. In some embodiments, R¹ is methyl. In some embodiments, R¹ is ethyl.

In some embodiments, A is selected from the group consisting of

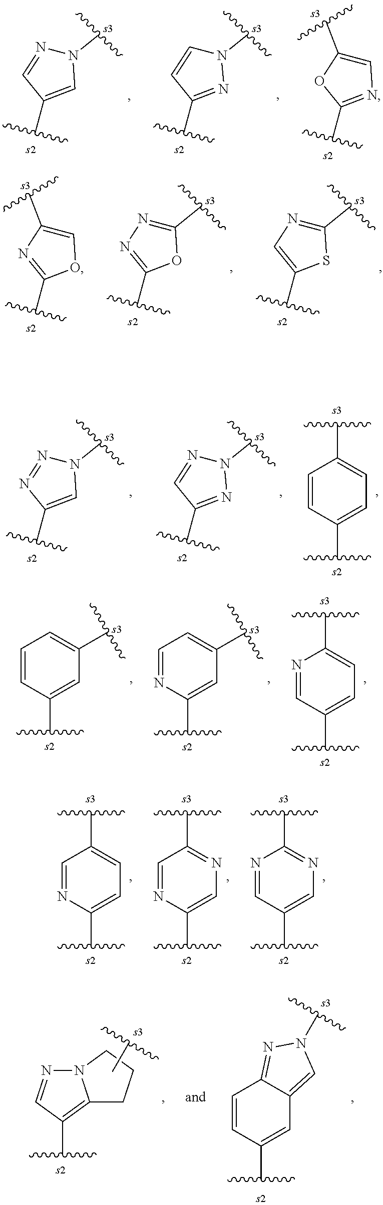

each of which is optionally substituted with alkyl, alkoxy, halogen, haloalkyl, haloalkoxy, hydroxy, or cyano, wherein s2 is the site covalently linked to —NH— and s3 is the site covalently linked to E.

In some embodiments, A is phenyl optionally substituted with halogen or alkyl.

In another embodiment, A is 5-6 membered heteroaryl optionally substituted with halogen or alkyl. In some embodiments, A is pyrazole optionally substituted with halogen or alkyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

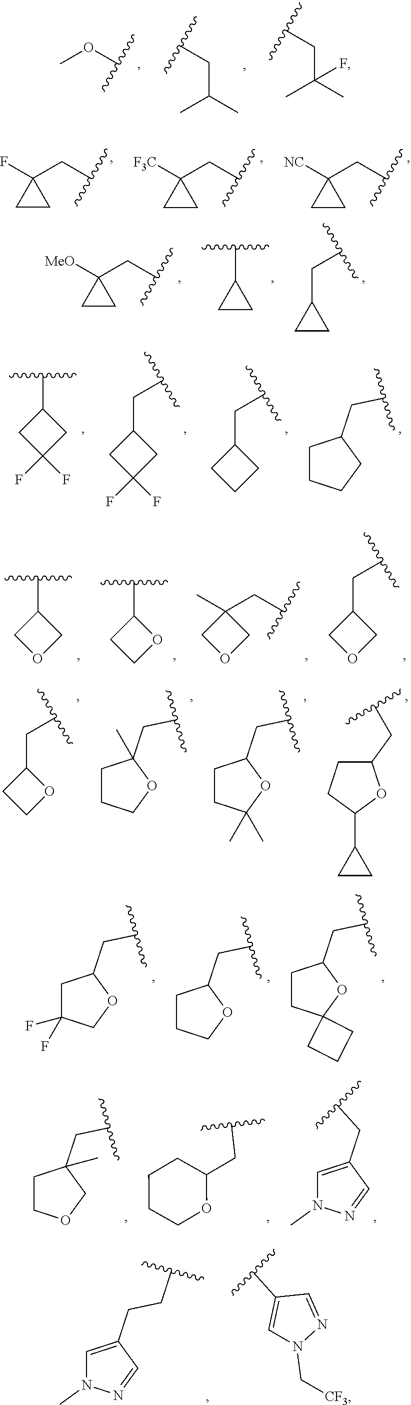

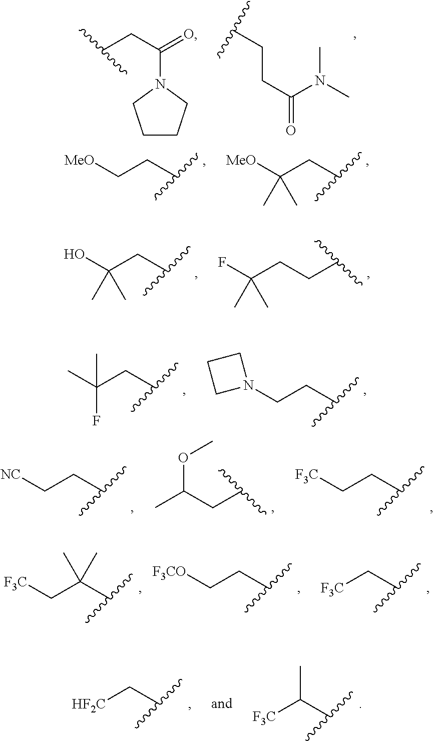

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

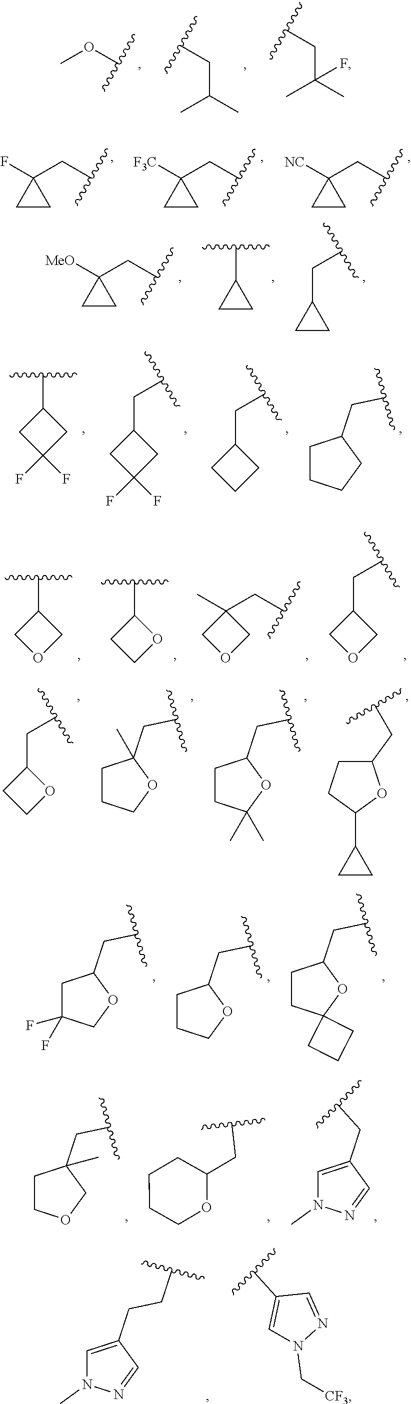

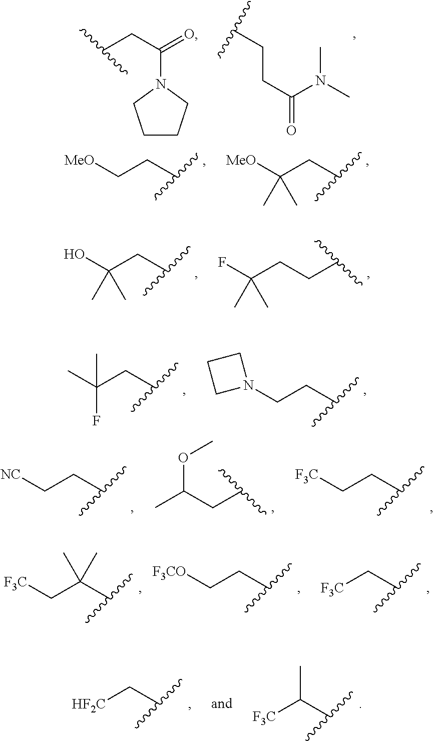

In some embodiments, R² and R³ together with the nitrogen atom to which they are attached form an optionally substituted heterocyclyl selected from the group consisting of

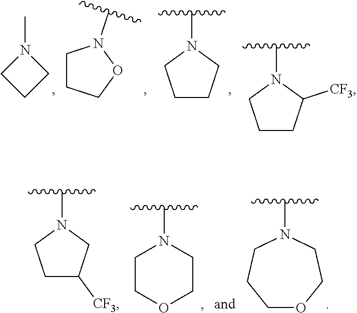

In some embodiments, R² and R³ together with the nitrogen atom to which they are attached form an optionally substituted heterocyclyl selected from the group consisting of

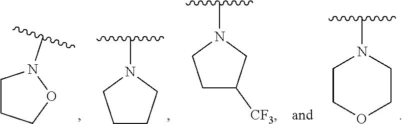

In some embodiments, E is selected from the group consisting of

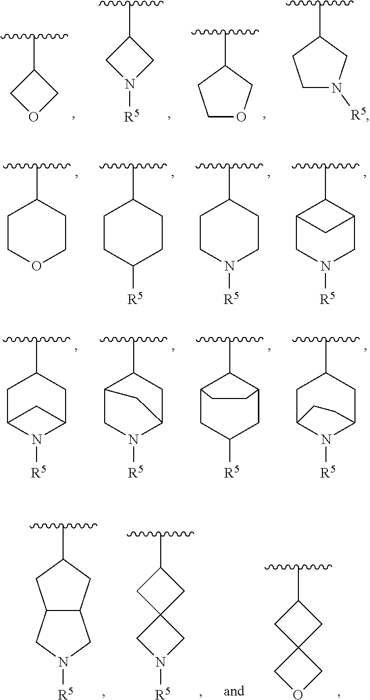

wherein R⁵ is independently H, alkyl, cycloalkyl, haloalkyl, or heterocyclyl.

In some embodiments, E is selected from the group consisting of

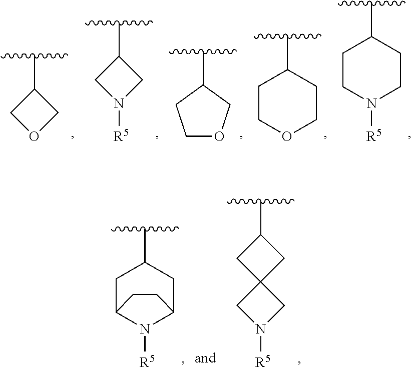

wherein R⁵ is independently H, alkyl, cycloalkyl, haloalkyl, or heterocyclyl.

In some embodiments, when A is pyrazole, E is selected from the group consisting of

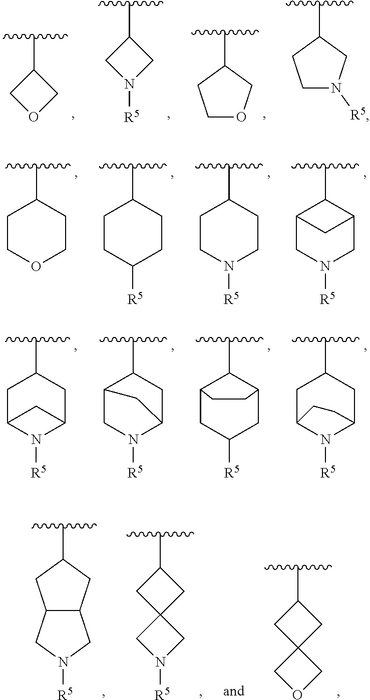

wherein R⁵ is independently H, alkyl, cycloalkyl, haloalkyl, or heterocyclyl.

In some embodiments, when A is pyrazole, E is selected from the group consisting of

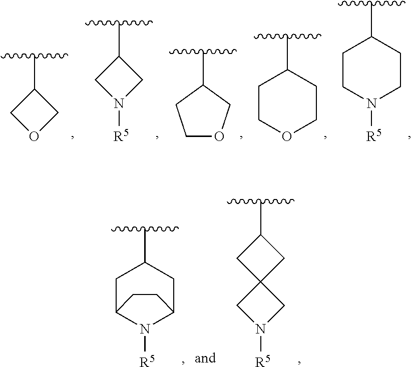

wherein R⁵ is independently H, alkyl, cycloalkyl, haloalkyl, or heterocyclyl.

In some embodiments, E is selected from the group consisting of

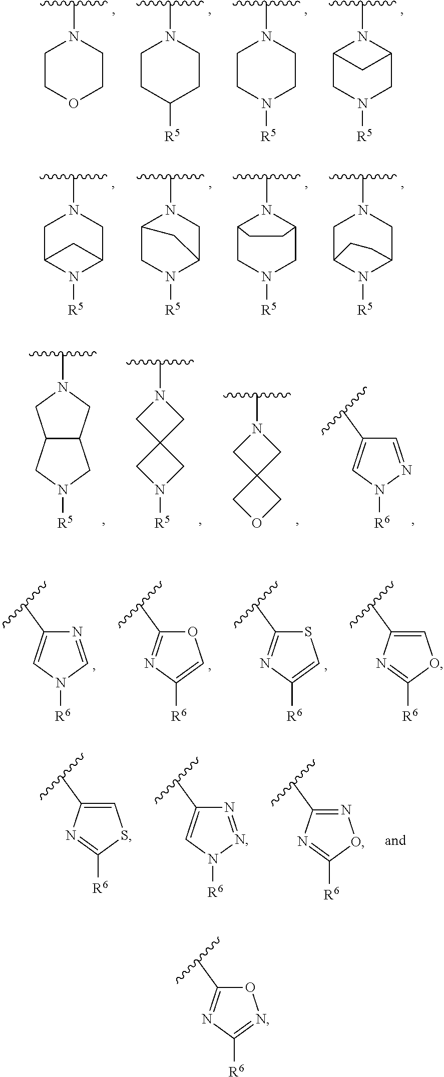

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

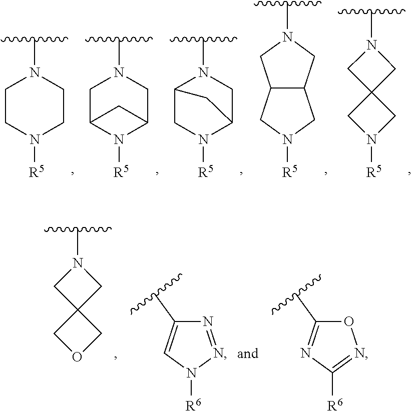

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

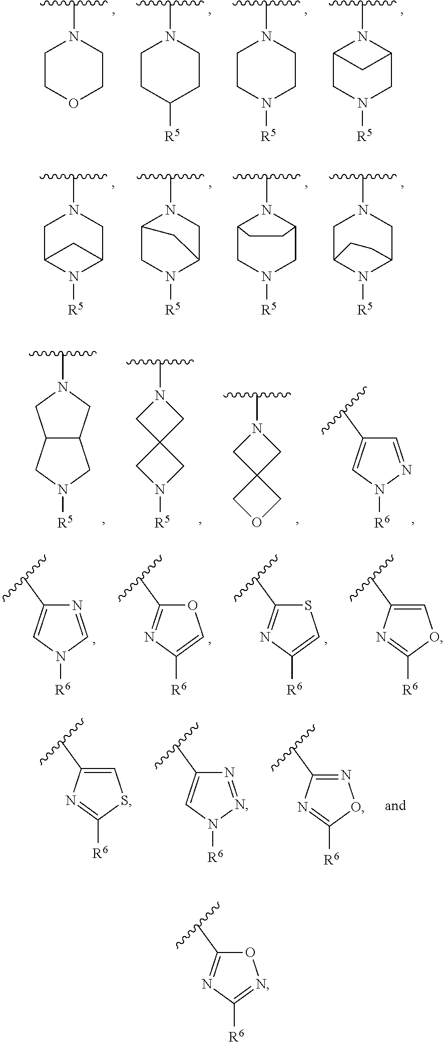

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

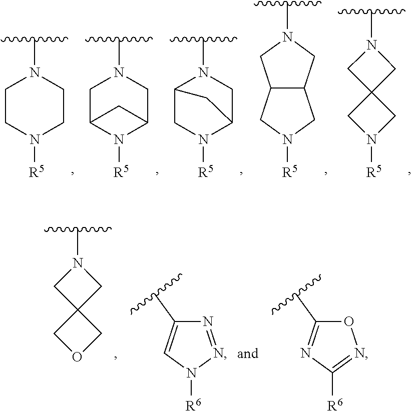

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

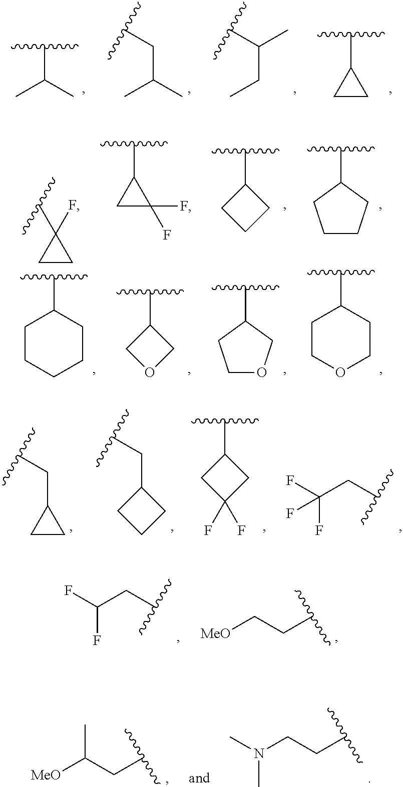

In some embodiments, E is selected from the group consisting of H, methyl, ethyl, trifluoromethyl, difluoromethyl,

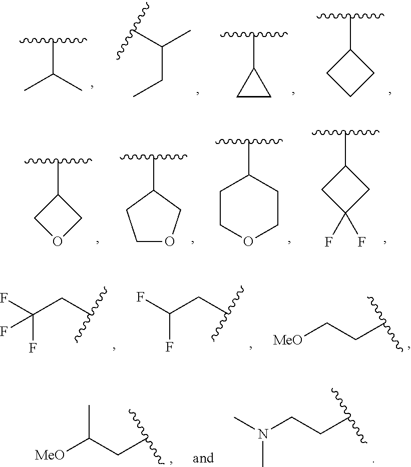

In one embodiment, described herein is a compound represented by Formula VII:

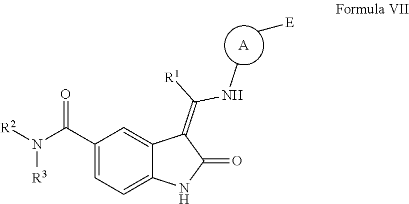

or a pharmaceutically acceptable salt, enantiomer, stereoisomer, or tautomer thereof, wherein:
A is selected from the group consisting of
   optionally substituted heteroaryl wherein the optionally substituted substituent, at each occurrence, is independently selected from the group consisting of alkyl, alkoxy, halogen, haloalkyl, haloalkoxy, hydroxy, and cyano, and
   optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano, provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, R¹ is alkyl, for example, methyl or ethyl. In some embodiments, R¹ is methyl. In some embodiments, R¹ is ethyl.

In some embodiments, A is selected from the group consisting of

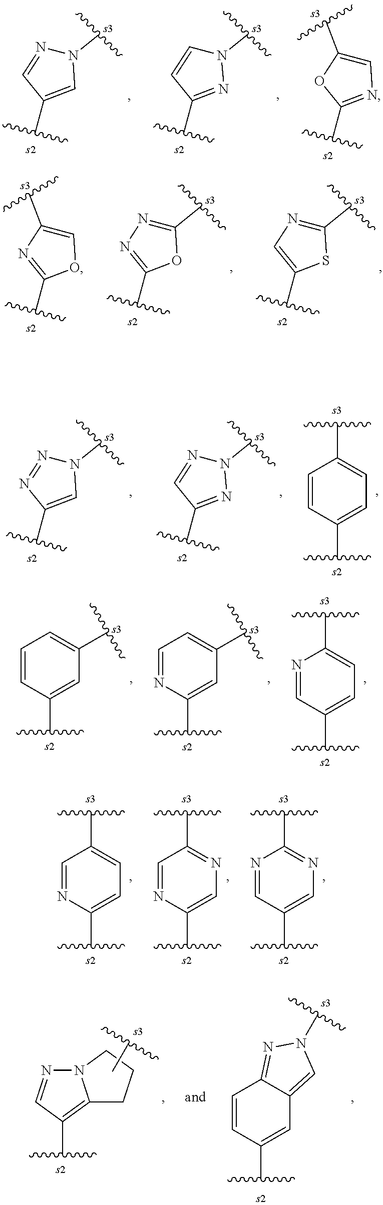

each of which is optionally substituted with alkyl, alkoxy, halogen, haloalkyl, haloalkoxy, hydroxy, or cyano, wherein s2 is the site covalently linked to —NH— and s3 is the site covalently linked to E.

In some embodiments, A is phenyl optionally substituted with halogen or alkyl.

In another embodiment, A is 5-6 membered heteroaryl optionally substituted with halogen or alkyl. In some embodiments, A is pyrazole optionally substituted with halogen or alkyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

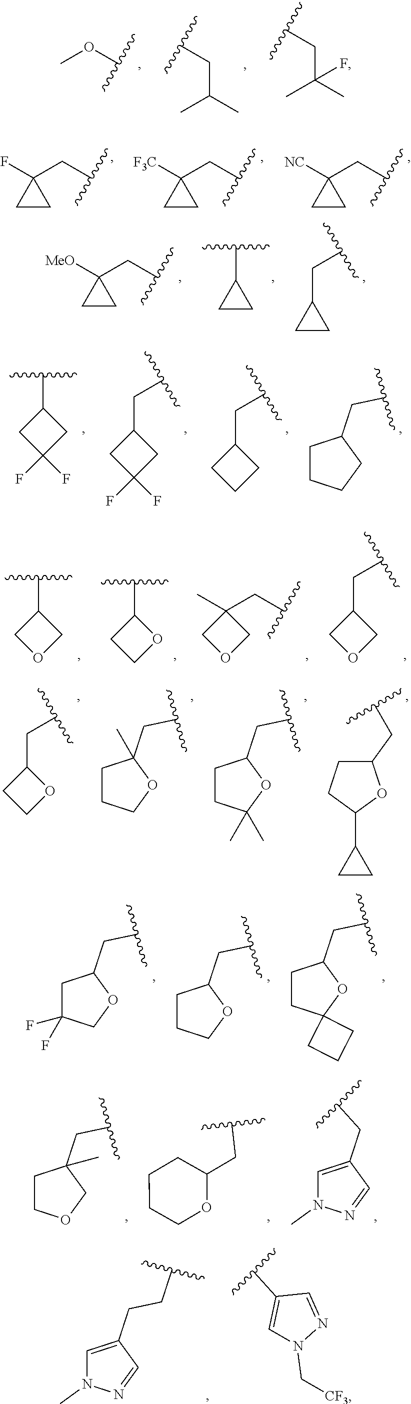

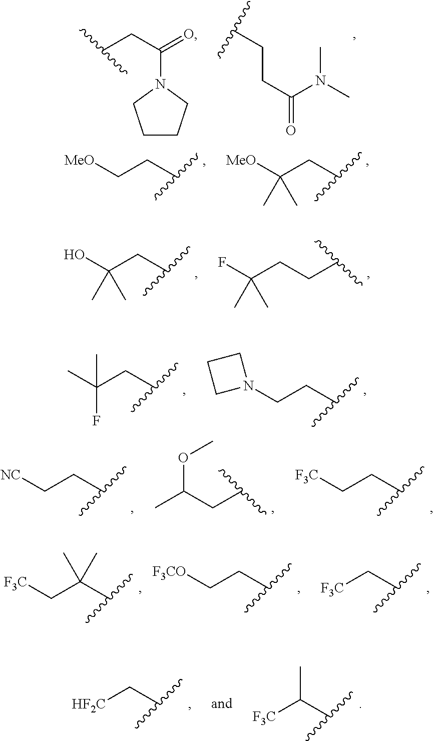

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

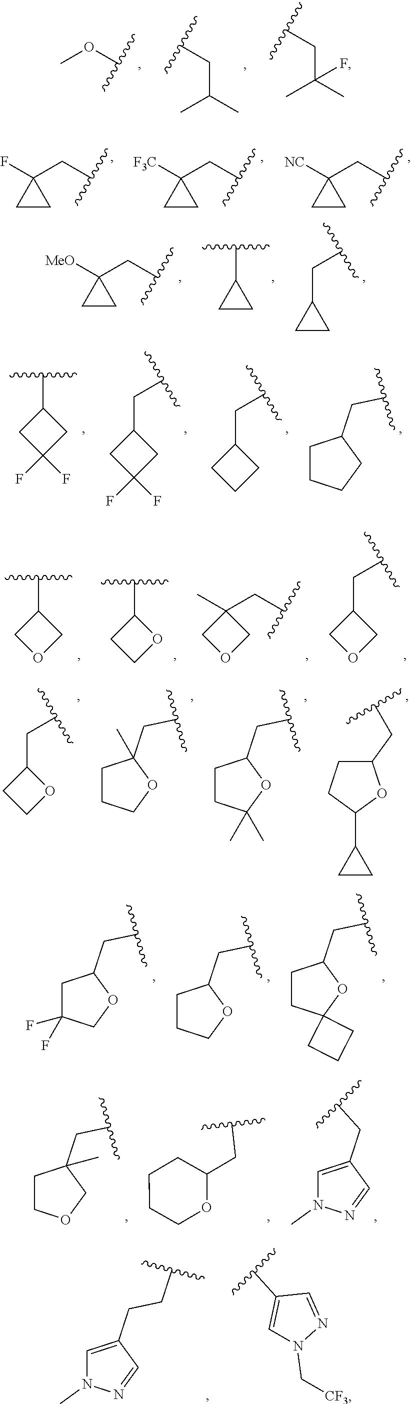

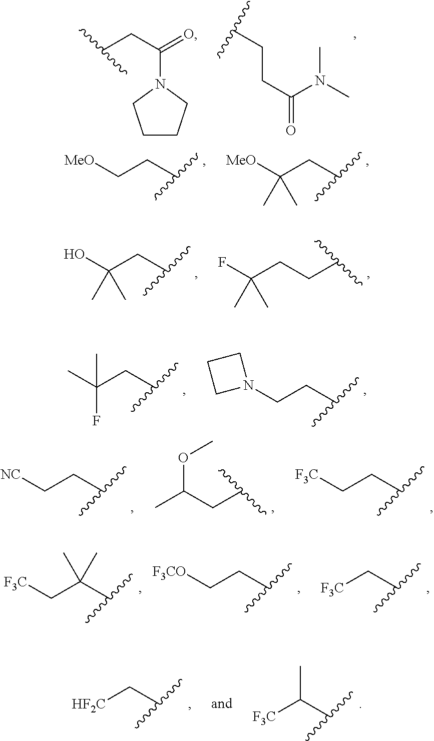

In some embodiments, R² and R³ together with the nitrogen atom to which they are attached form an optionally substituted heterocyclyl selected from the group consisting of

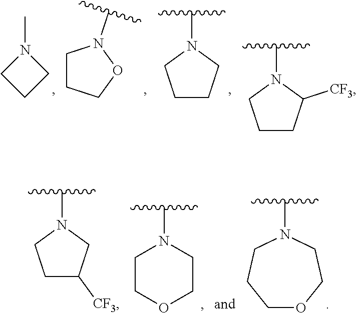

In some embodiments, R² and R³ together with the nitrogen atom to which they are attached form an optionally substituted heterocyclyl selected from the group consisting of

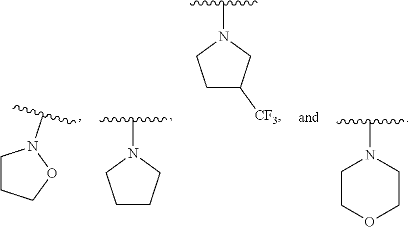 [SMILES: *N1CCCO1 ; *N1CCCC1 ; *N1CCC(C(F)(F)F)C1 ; *N1CCOCC1]

In some embodiments, E is selected from the group consisting of

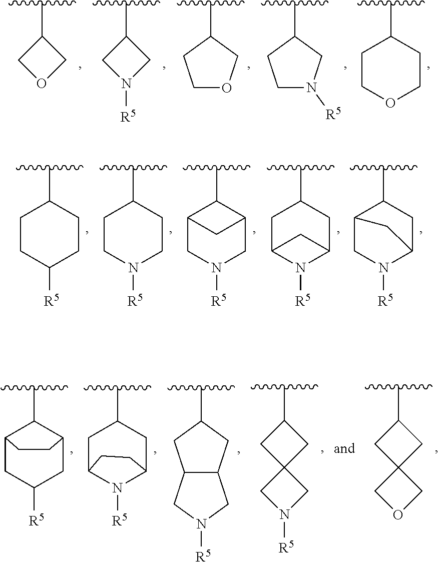

wherein R⁵ is independently H, alkyl, cycloalkyl, haloalkyl, or heterocyclyl.

In some embodiments, E is selected from the group consisting of

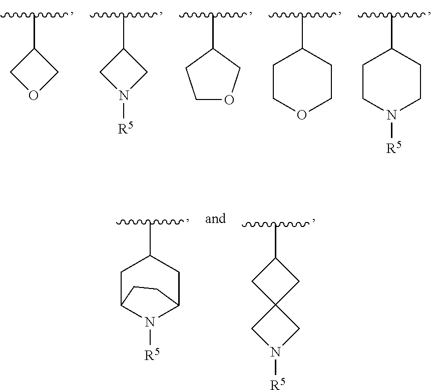

wherein R⁵ is independently H, alkyl, cycloalkyl, haloalkyl, or heterocyclyl.

In some embodiments, when A is pyrazole, E is selected from the group consisting of

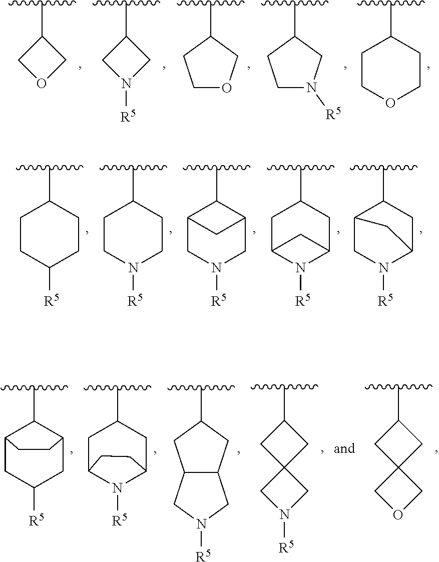

wherein R⁵ is independently H, alkyl, cycloalkyl, haloalkyl, or heterocyclyl.

In some embodiments, when A is pyrazole, E is selected from the group consisting of

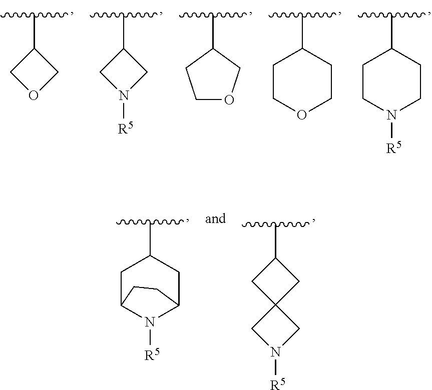

wherein R⁵ is independently H, alkyl, cycloalkyl, haloalkyl, or heterocyclyl.

In some embodiments, E is selected from the group consisting of

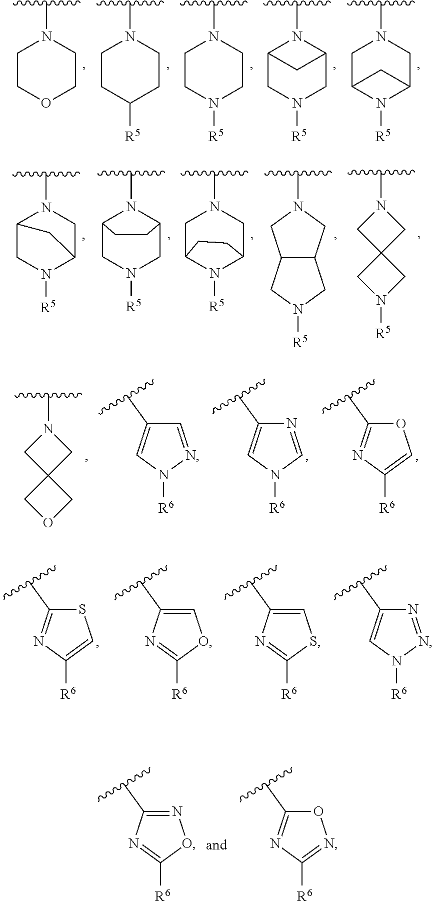

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

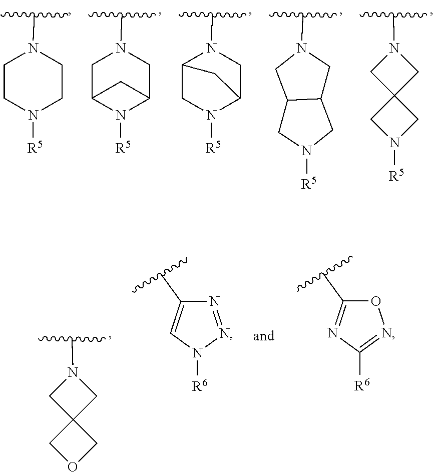

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

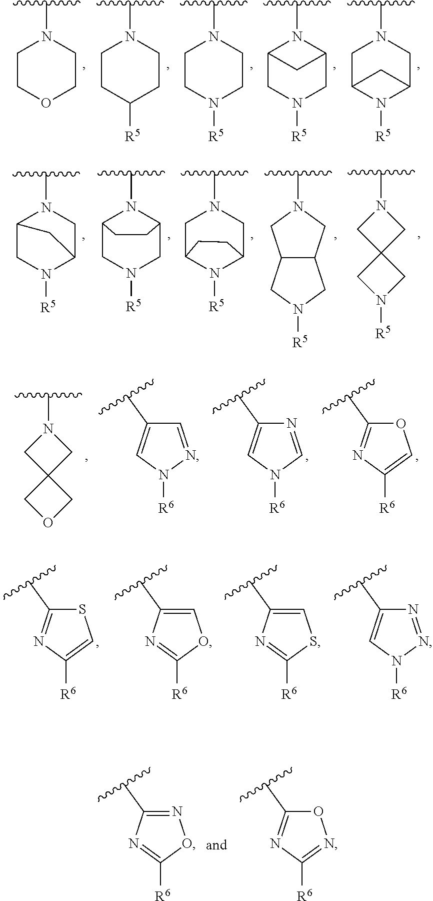

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

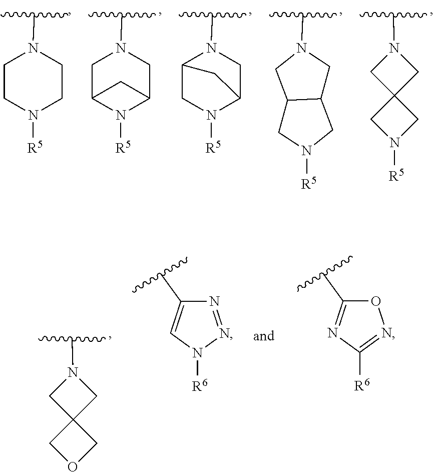

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

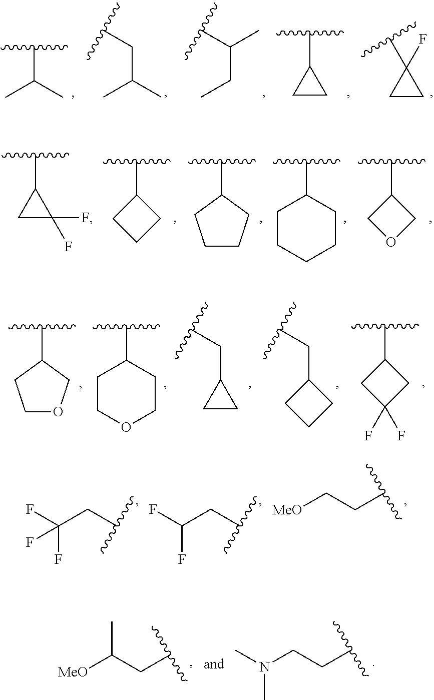

In some embodiments, E is selected from the group consisting of H, methyl, ethyl, trifluoromethyl, difluoromethyl,

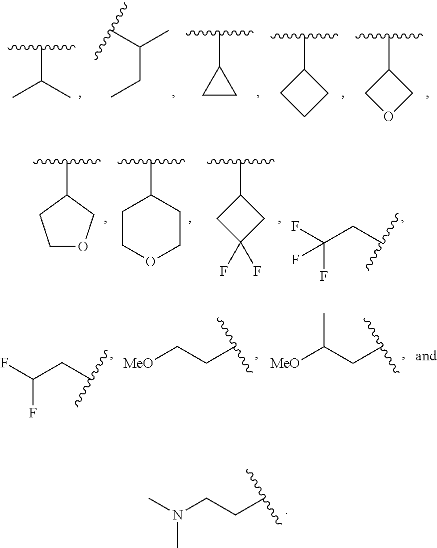

In another embodiment, described herein is a compound represented by Formula VIII:

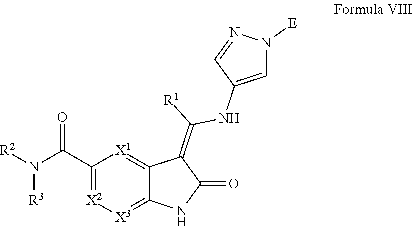

or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
   provided that not more than two of X¹, X², and X³ are N;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
   R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, X¹ is CH. In other embodiments, X¹ is CF or N. In some embodiments, X³ is CH. In other embodiments, X³ is CF or N. In some embodiments, X² is CH. In other embodiments, X² is CF, C-Cl, or N. In some embodiments, X¹, X², and X³ are CH.

In some embodiments, R¹ is alkyl, for example, methyl or ethyl. In some embodiments, R¹ is methyl. In some embodiments, R¹ is ethyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

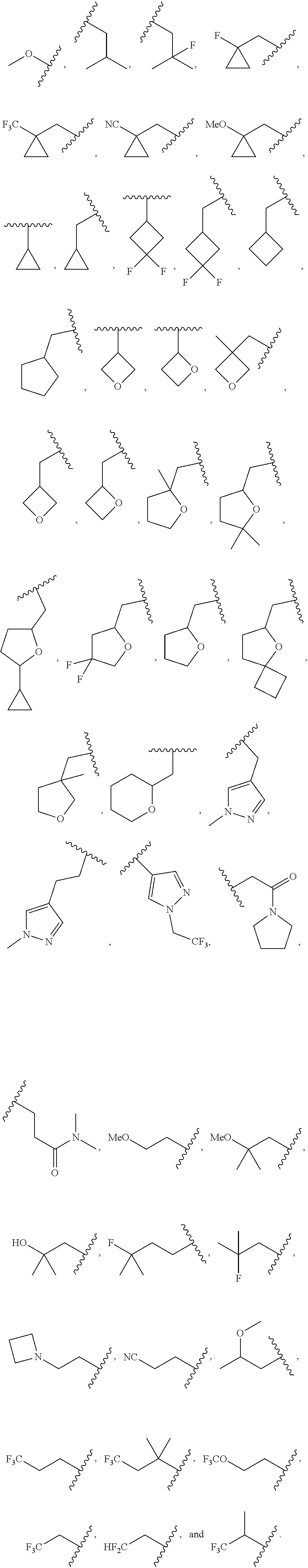

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

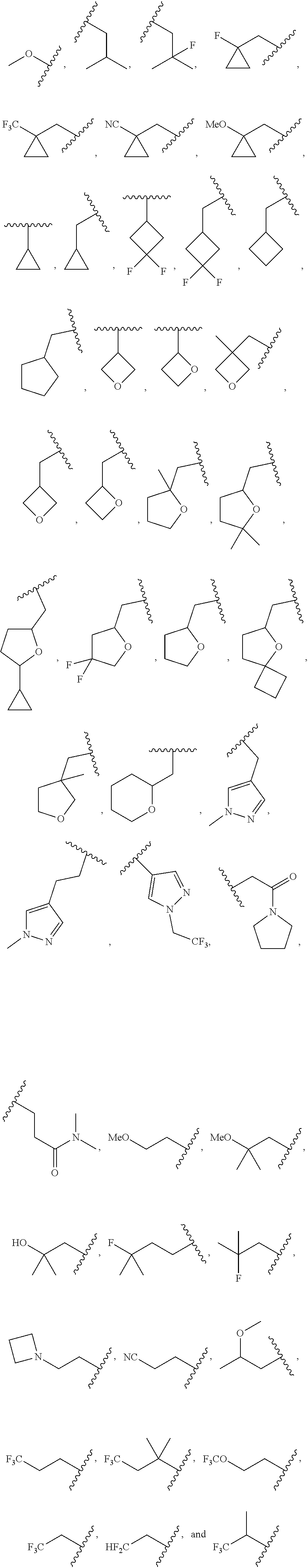

In some embodiments, R² and R³ together with the nitrogen atom to which they are attached form an optionally substituted heterocyclyl selected from the group consisting of

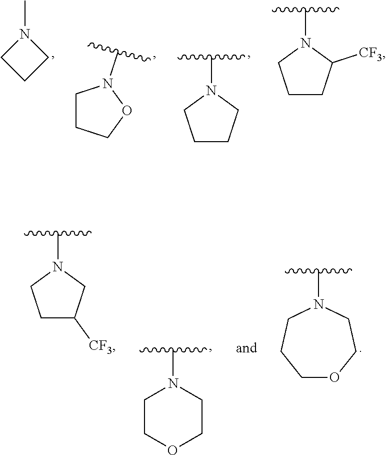

In some embodiments, R² and R³ together with the nitrogen atom to which they are attached form an optionally substituted heterocyclyl selected from the group consisting of

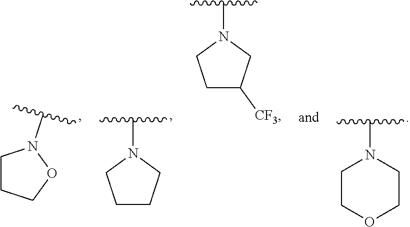 [SMILES: *N1CCCO1 ; *N1CCCC1 ; *N1CCC(C(F)(F)F)C1 ; *N1CCOCC1]

In some embodiments, E is selected from the group consisting of

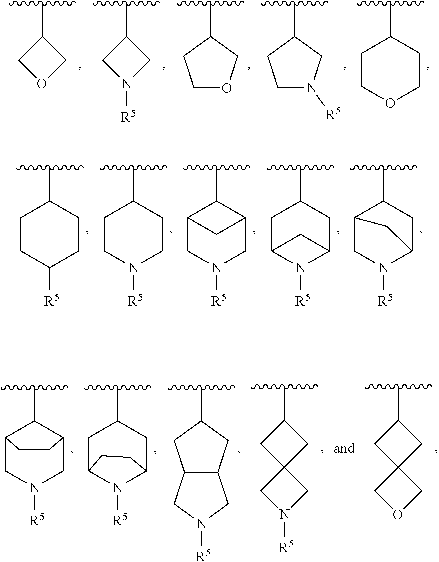

wherein R⁵ is independently H, alkyl, cycloalkyl, haloalkyl, or heterocyclyl.

In some embodiments, E is selected from the group consisting of

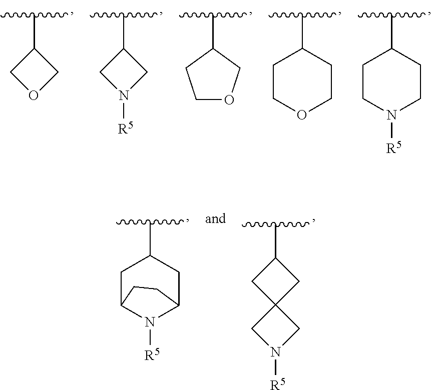

wherein R⁵ is independently H, alkyl, cycloalkyl, haloalkyl, or heterocyclyl.

In some embodiments, E is selected from the group consisting of

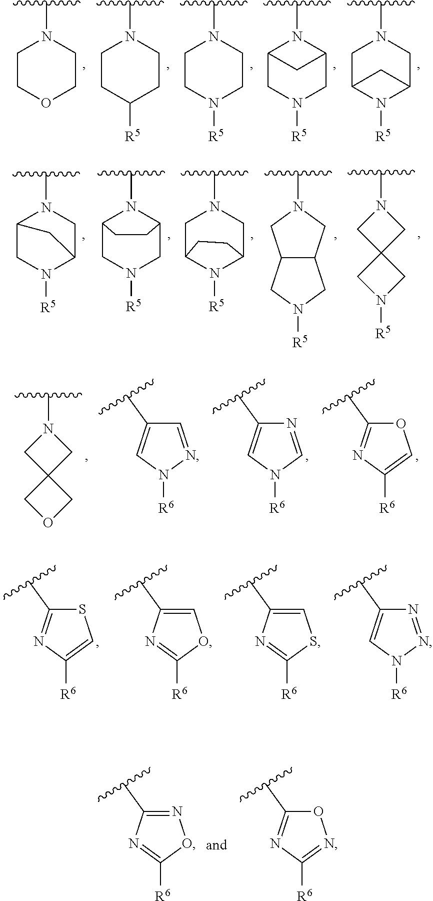

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

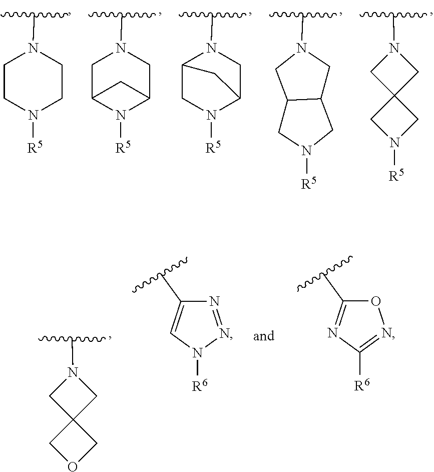

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

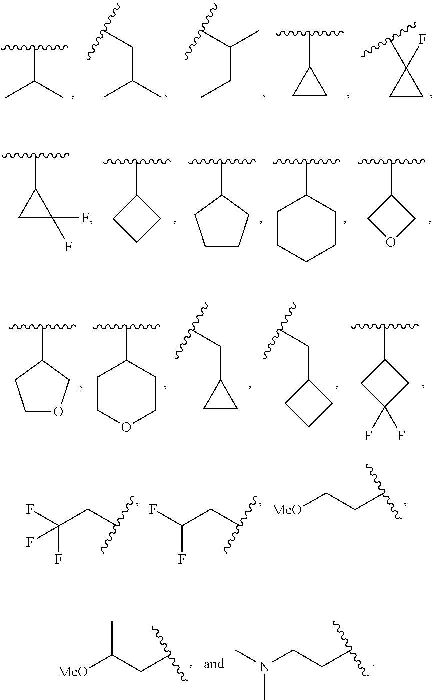

In some embodiments, E is selected from the group consisting of H, methyl, ethyl, trifluoromethyl, difluoromethyl,

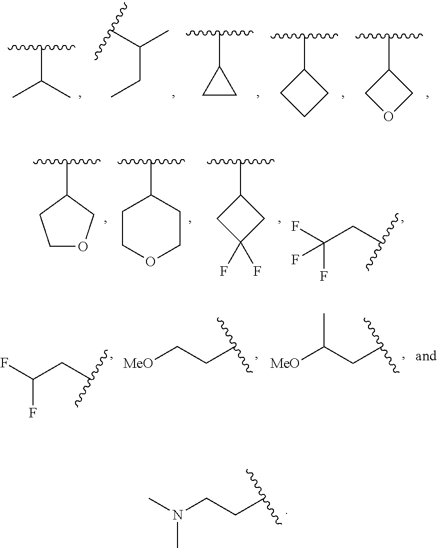

In one embodiment, described herein is a compound represented by Formula IX:

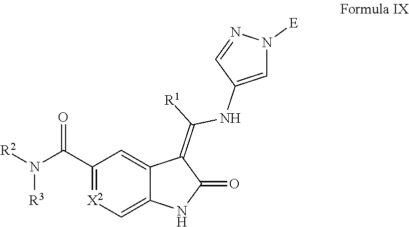

or a pharmaceutically acceptable salt, enantiomer, stereoisomer, or tautomer thereof, wherein:
X² is selected from the group consisting of N, CH, CF, and C-Cl;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
   R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, X² is CH. In other embodiments, X² is CF, C-Cl, or N.

In some embodiments, R¹ is alkyl, for example, methyl or ethyl. In some embodiments, R¹ is methyl. In some embodiments, R¹ is ethyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

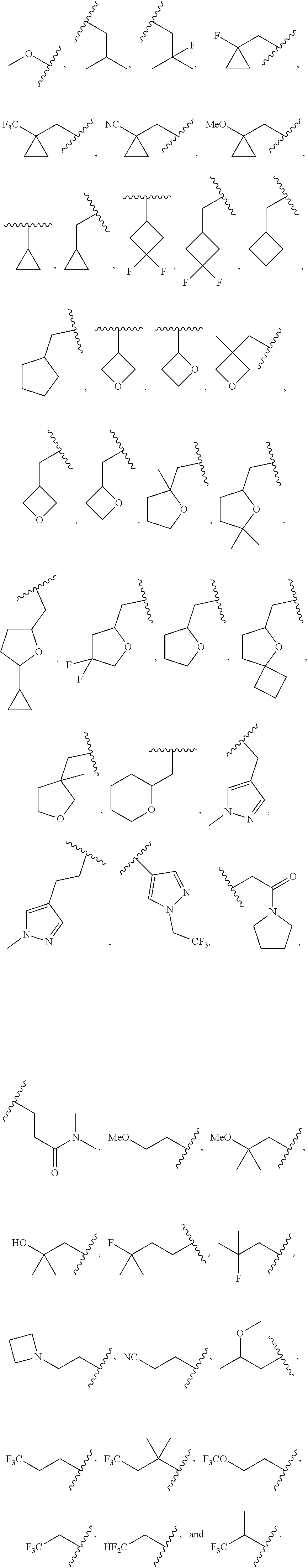

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

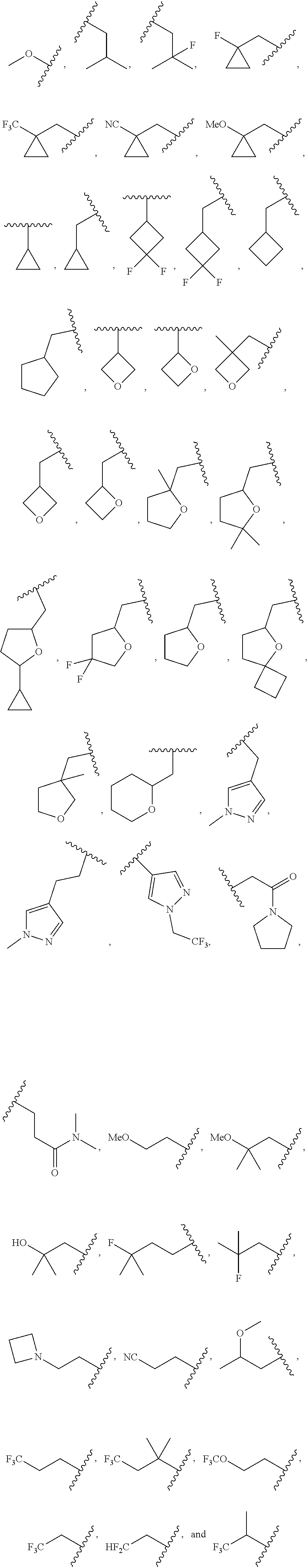

In some embodiments, R² and R³ together with the nitrogen atom to which they are attached form an optionally substituted heterocyclyl selected from the group consisting of

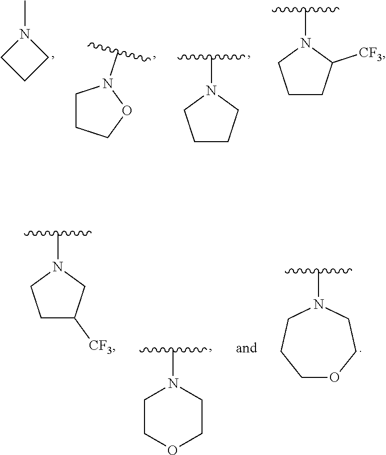

In some embodiments, R² and R³ together with the nitrogen atom to which they are attached form an optionally substituted heterocyclyl selected from the group consisting of

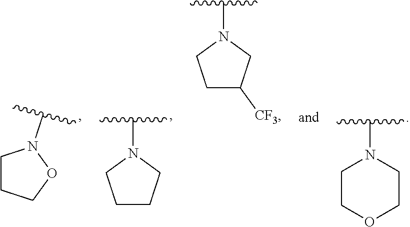 [SMILES: *N1CCCO1 ; *N1CCCC1 ; *N1CCC(C(F)(F)F)C1 ; *N1CCOCC1]

In some embodiments, E is selected from the group consisting of

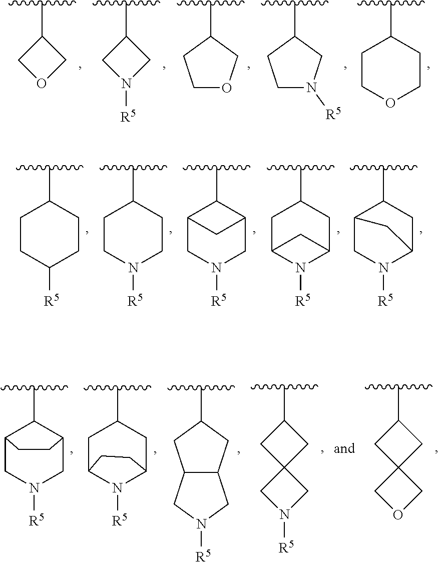

wherein R⁵ is independently H, alkyl, cycloalkyl, haloalkyl, or heterocyclyl.

In some embodiments, E is selected from the group consisting of

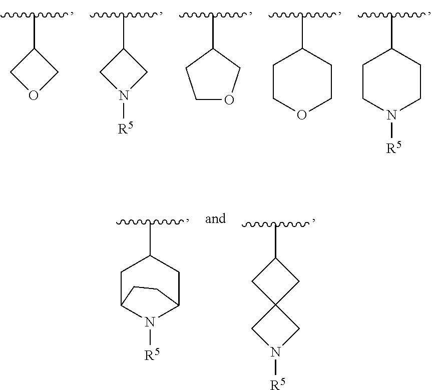

wherein R⁵ is independently H, alkyl, cycloalkyl, haloalkyl, or heterocyclyl.

In some embodiments, E is selected from the group consisting of

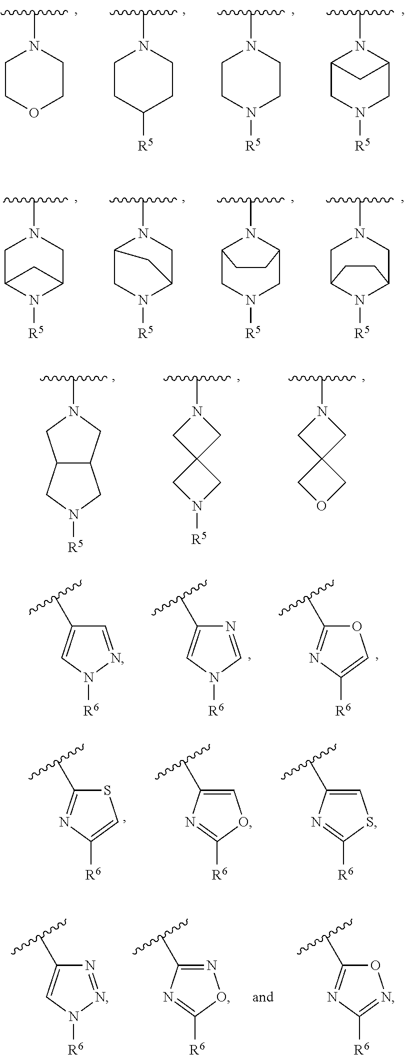

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

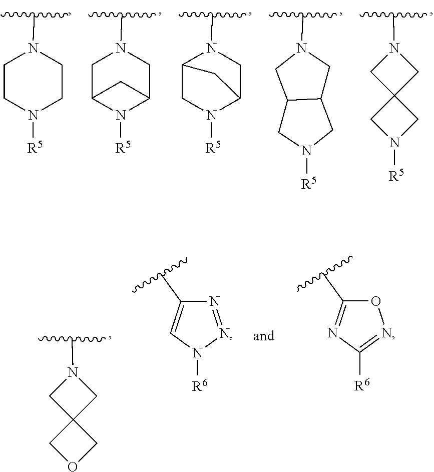

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

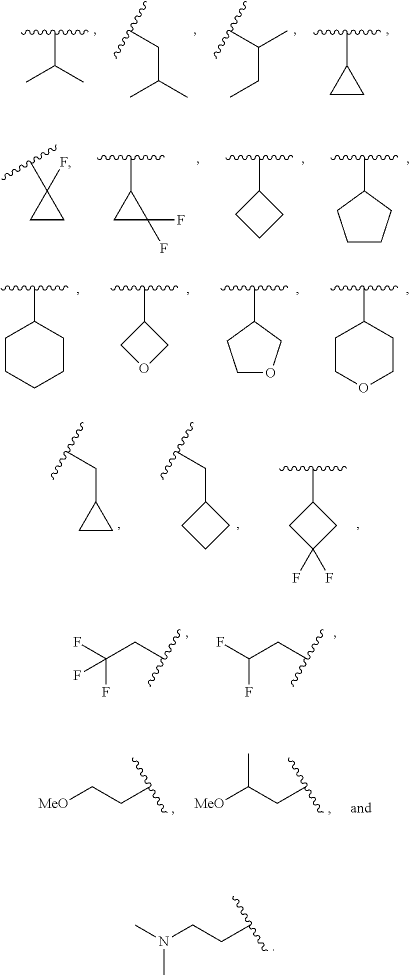

In some embodiments, E is selected from the group consisting of H, methyl, ethyl, trifluoromethyl, difluoromethyl,

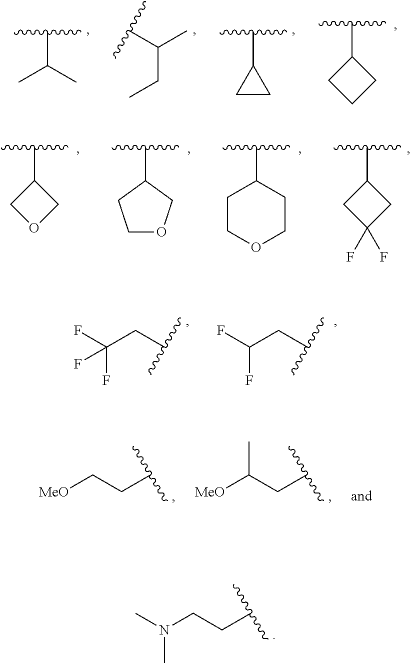

In another embodiment, described herein is a compound represented by Formula X:

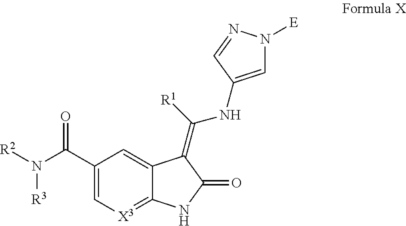

or a pharmaceutically acceptable salt, enantiomer, stereoisomer, or tautomer thereof, wherein:
X³ is selected from the group consisting of N, CH, and CF;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
   R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, X³ is CH. In other embodiments, X³ is CF or N.

In some embodiments, R¹ is alkyl, for example, methyl or ethyl. In some embodiments, R¹ is methyl. In some embodiments, R¹ is ethyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

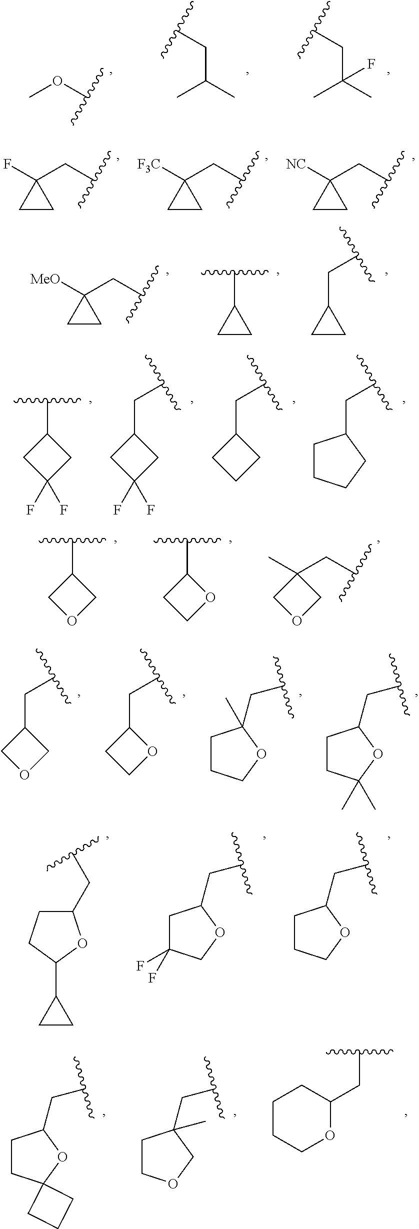

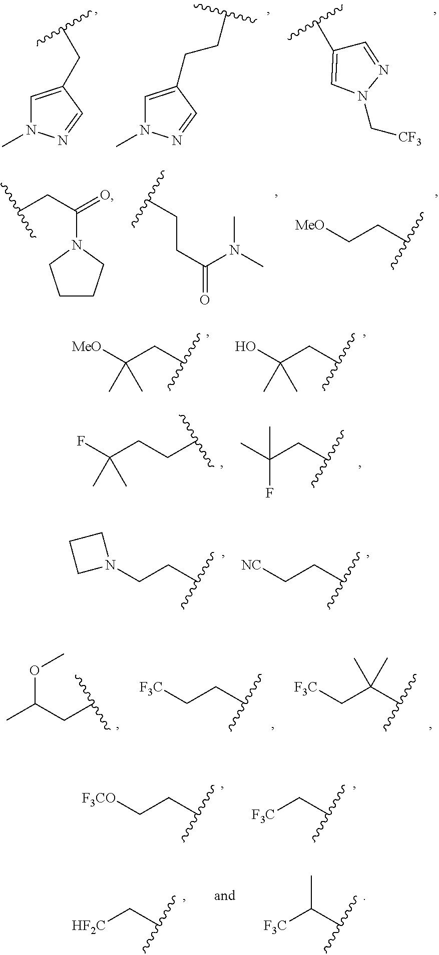

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

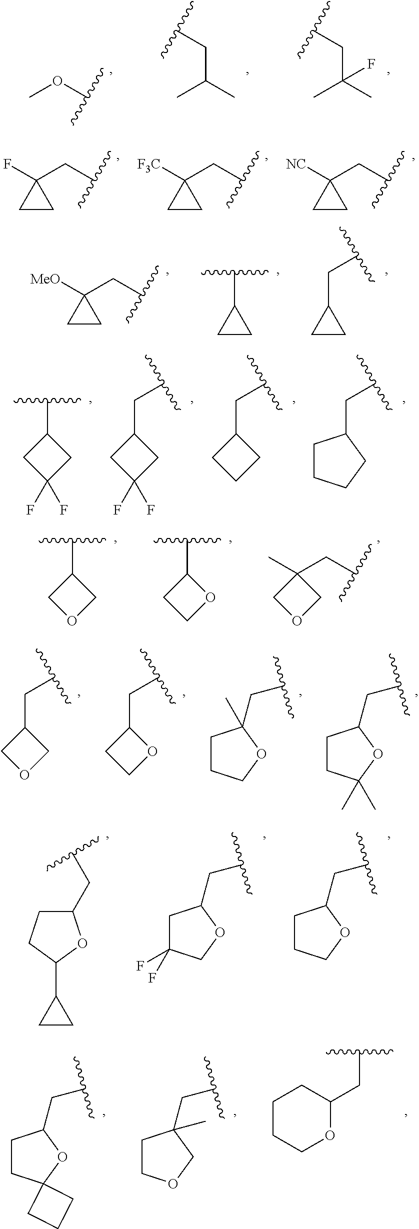

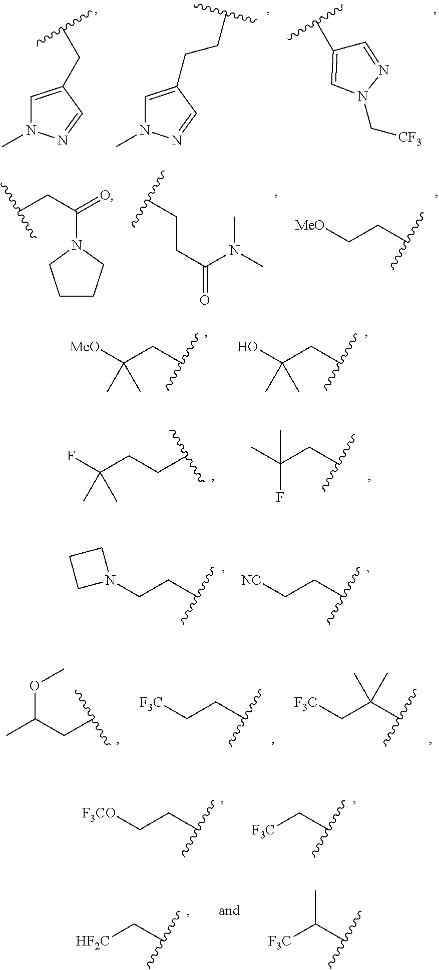

In some embodiments, R² and R³ together with the nitrogen atom to which they are attached form an optionally substituted heterocyclyl selected from the group consisting of

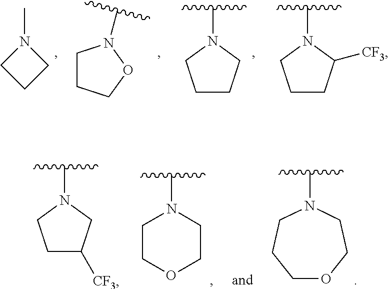

In some embodiments, R² and R³ together with the nitrogen atom to which they are attached form an optionally substituted heterocyclyl selected from the group consisting of

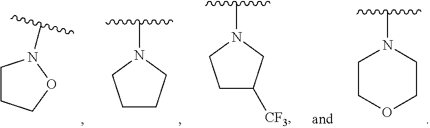 [SMILES: *N1CCCO1 ; *N1CCCC1 ; *N1CCC(C(F)(F)F)C1 ; *N1CCOCC1]

In some embodiments, E is selected from the group consisting of

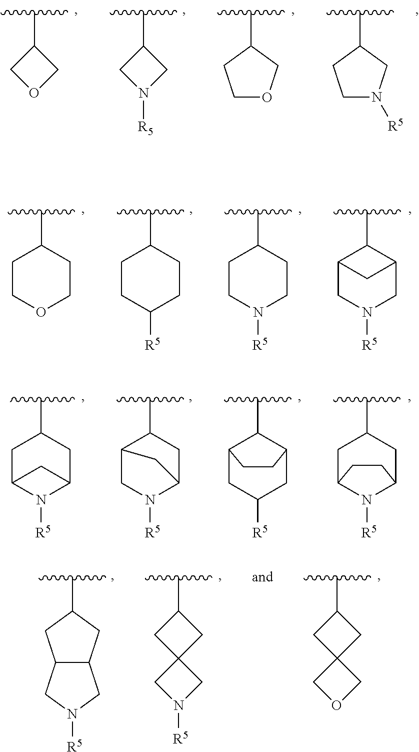

wherein R⁵ is independently H, alkyl, cycloalkyl, haloalkyl, or heterocyclyl.

In some embodiments, E is selected from the group consisting of

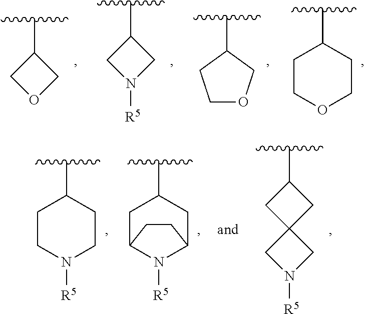

wherein R is independently H, alkyl, cycloalkyl, haloalkyl, or heterocyclyl.

In some embodiments, E is selected from the group consisting of

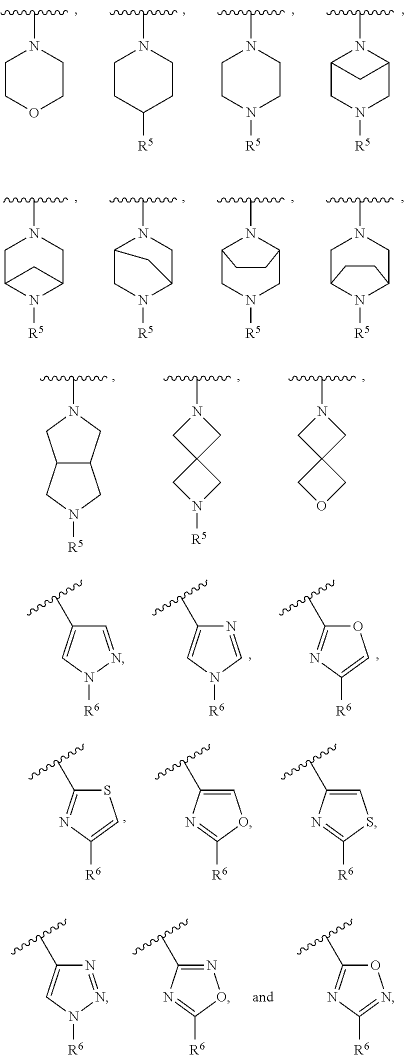

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

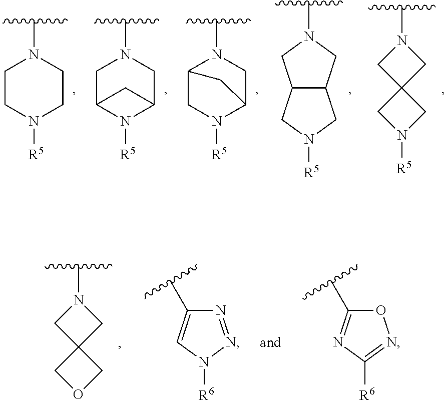

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

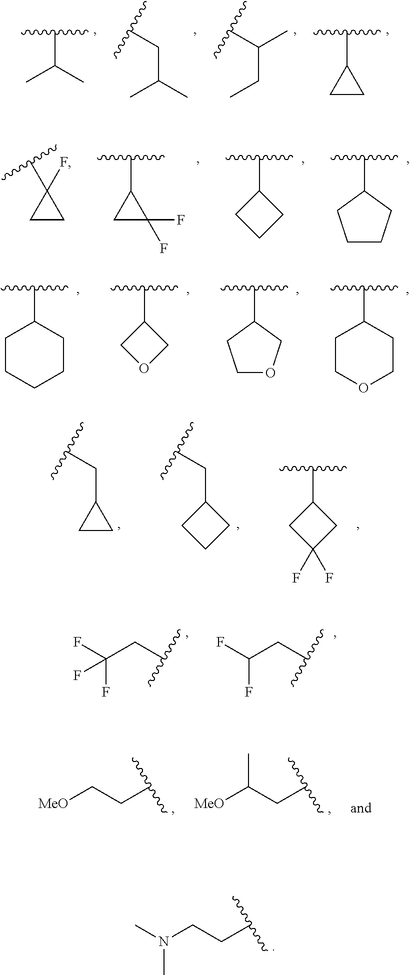

In some embodiments, E is selected from the group consisting of H, methyl, ethyl, trifluoromethyl, difluoromethyl,

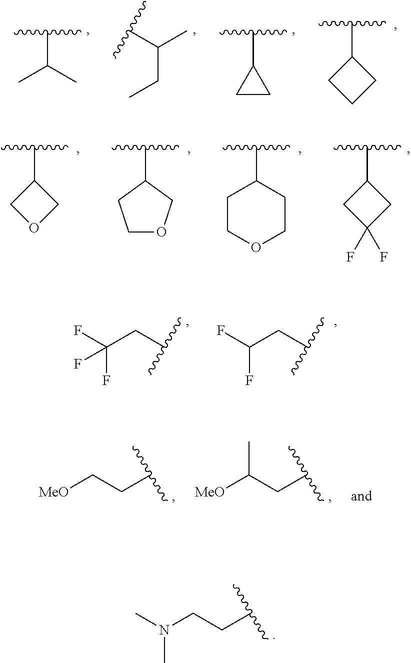

In another embodiment, described herein is a compound represented by Formula XI:

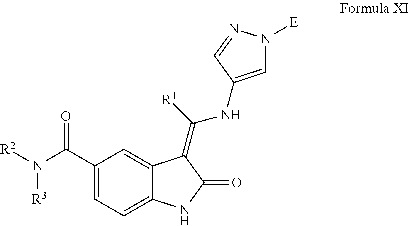

or a pharmaceutically acceptable salt, enantiomer, stereoisomer, or tautomer thereof, wherein:
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
   R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, R¹ is alkyl, for example, methyl or ethyl. In some embodiments, R¹ is methyl. In some embodiments, R¹ is ethyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

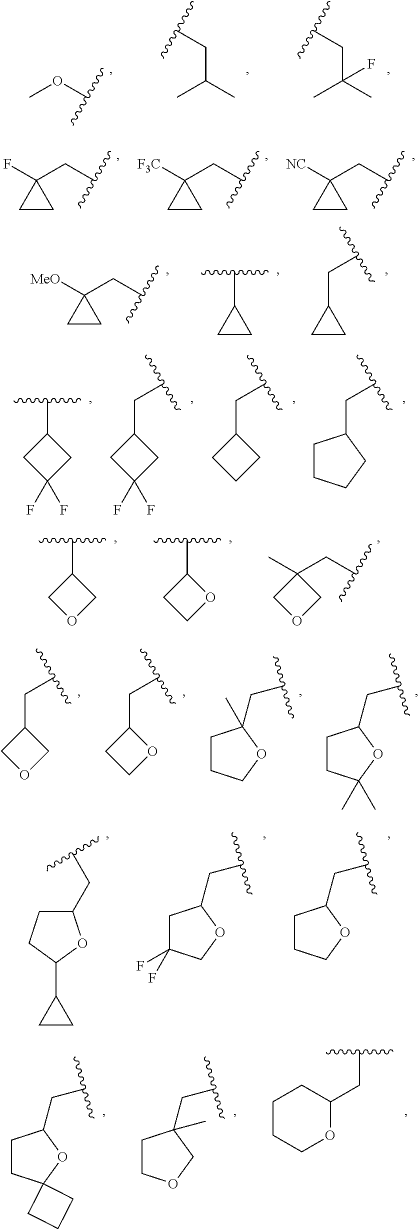

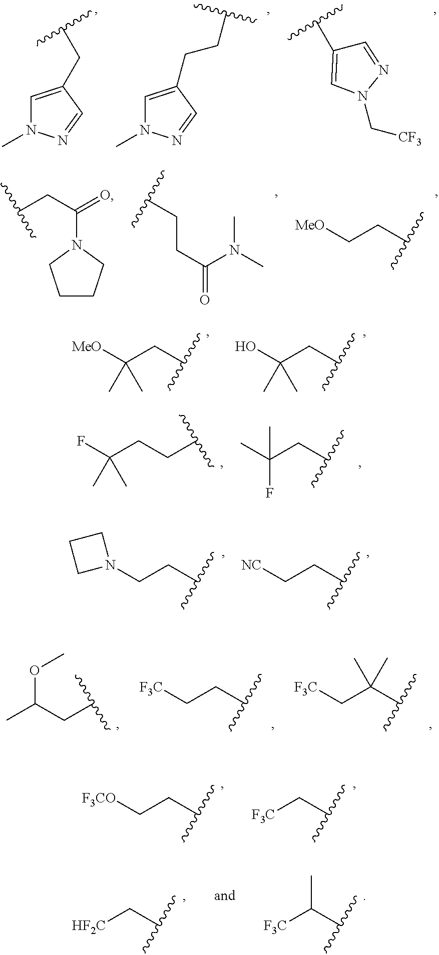

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

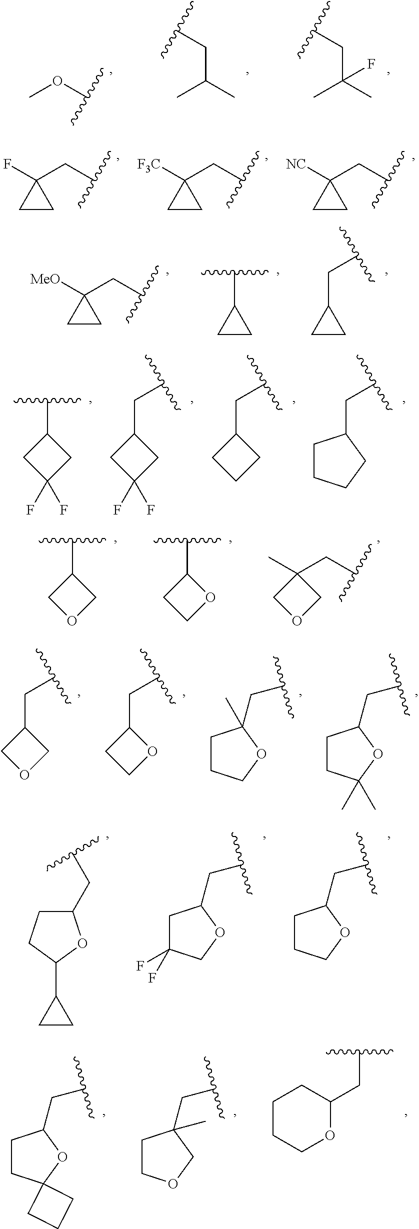

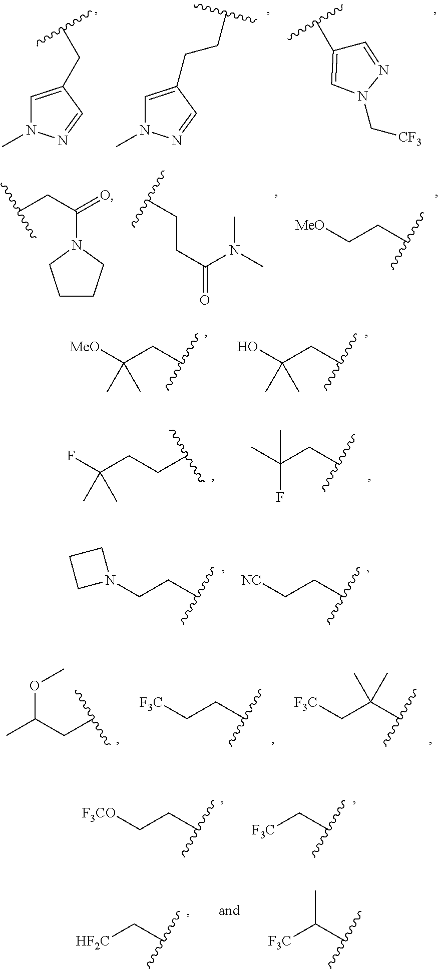

In some embodiments, R² and R³ together with the nitrogen atom to which they are attached form an optionally substituted heterocyclyl selected from the group consisting of

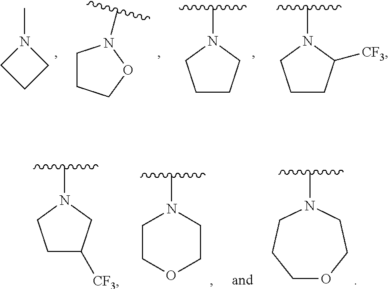

In some embodiments, R² and R³ together with the nitrogen atom to which they are attached form an optionally substituted heterocyclyl selected from the group consisting of

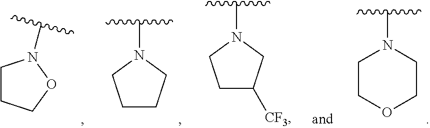 [SMILES: *N1CCCO1 ; *N1CCCC1 ; *N1CCC(C(F)(F)F)C1 ; *N1CCOCC1]

In some embodiments, E is selected from the group consisting of

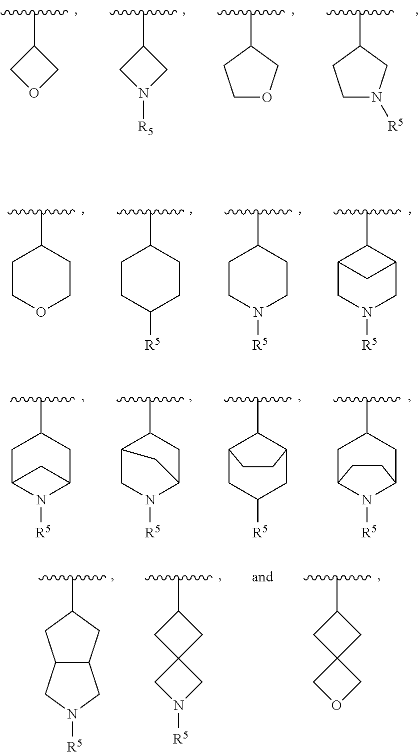

wherein R⁵ is independently H, alkyl, cycloalkyl, haloalkyl, or heterocyclyl.

In some embodiments, E is selected from the group consisting of

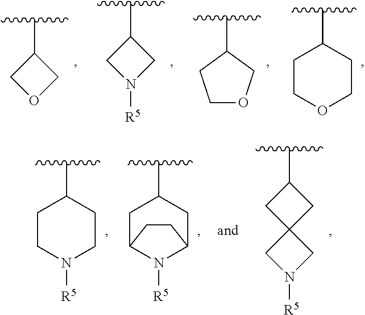

wherein R⁵ is independently H, alkyl, cycloalkyl, haloalkyl, or heterocyclyl.

In some embodiments, E is selected from the group consisting of

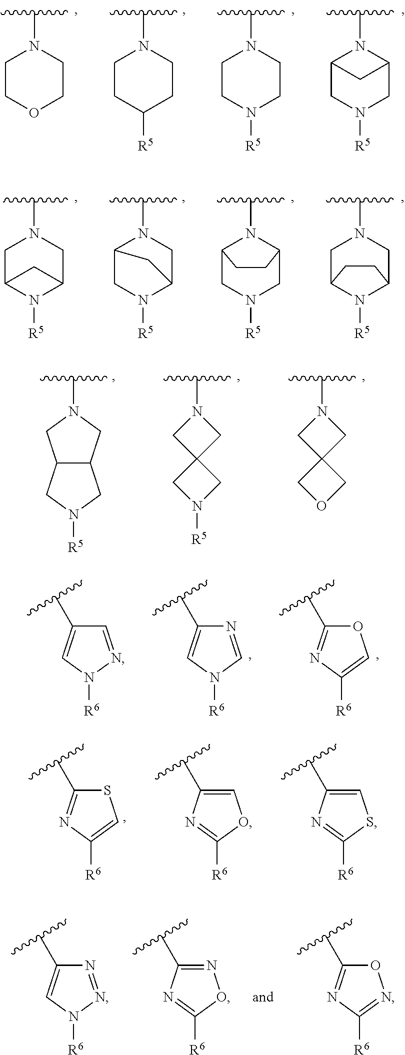

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

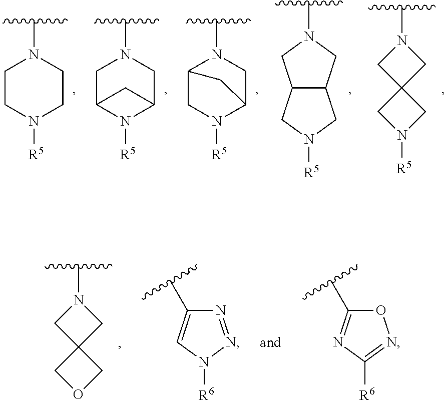

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

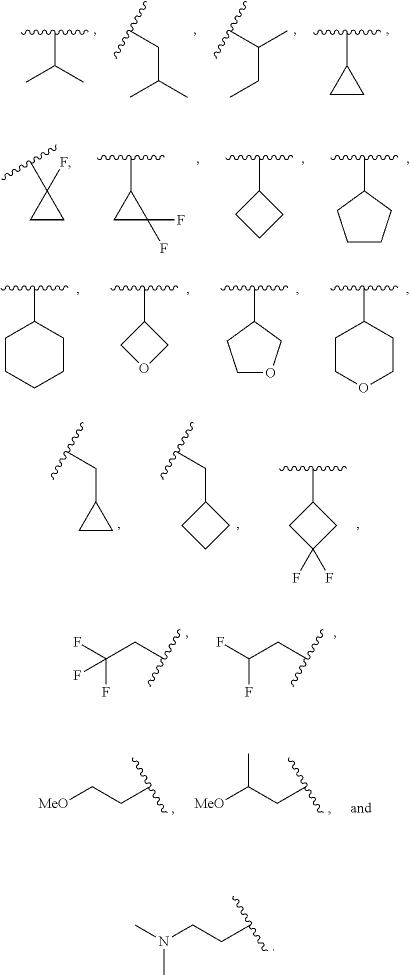

In some embodiments, E is selected from the group consisting of H, methyl, ethyl, trifluoromethyl, difluoromethyl,

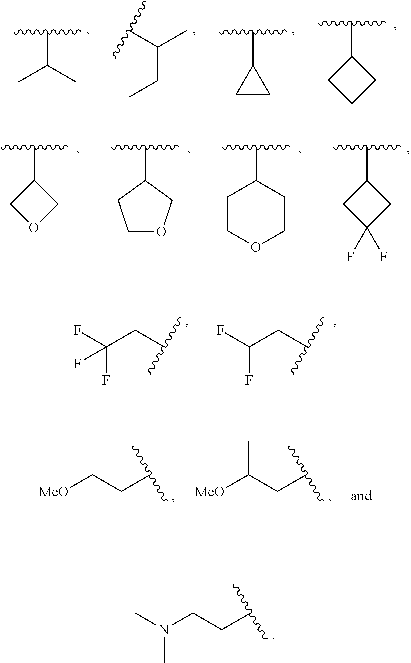

In another embodiment, described herein is a compound represented by Formula XII:

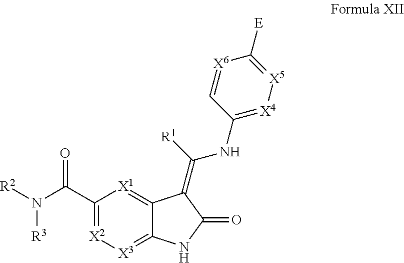

or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
   provided that not more than two of X¹, X², and X³ are N;
X⁴, X⁵, and X⁶ are each independently selected from the group consisting of N, CH, and CF;
provided that not more than two of X⁴, X⁵, and X⁶ are N:
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano,

In some embodiments, X¹ is CH. In other embodiments, X¹ is CF or N. In some embodiments, X³ is CH. In other embodiments, X³ is CF or N. In some embodiments, X² is CH. In other embodiments, X² is CF, C-Cl, or N. In some embodiments, X¹, X², and X³ are CH.

In some embodiments, R¹ is alkyl, for example, methyl or ethyl. In some embodiments, R¹ is methyl. In embodiments, R¹ is ethyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

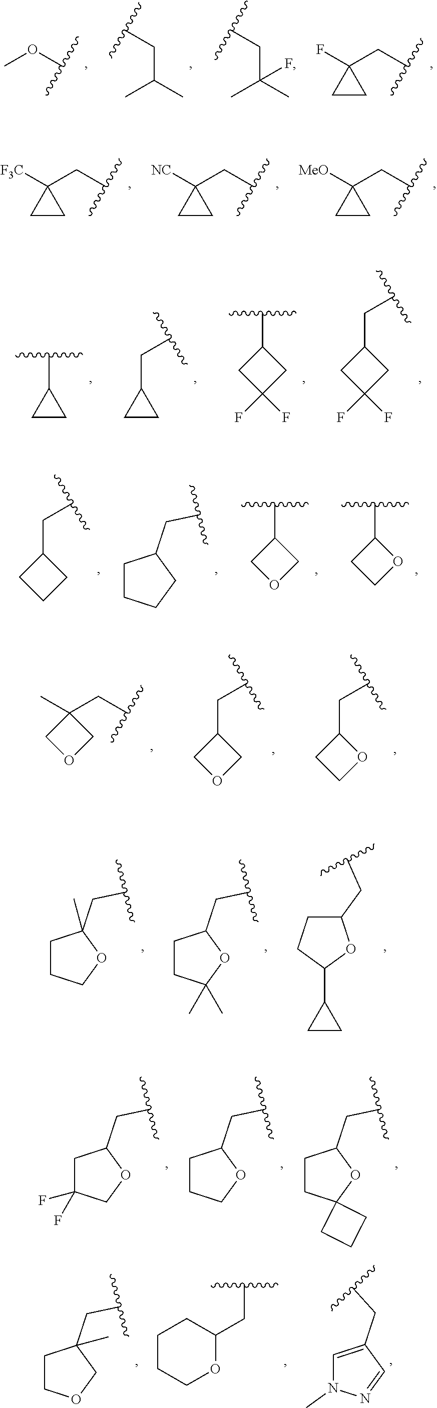

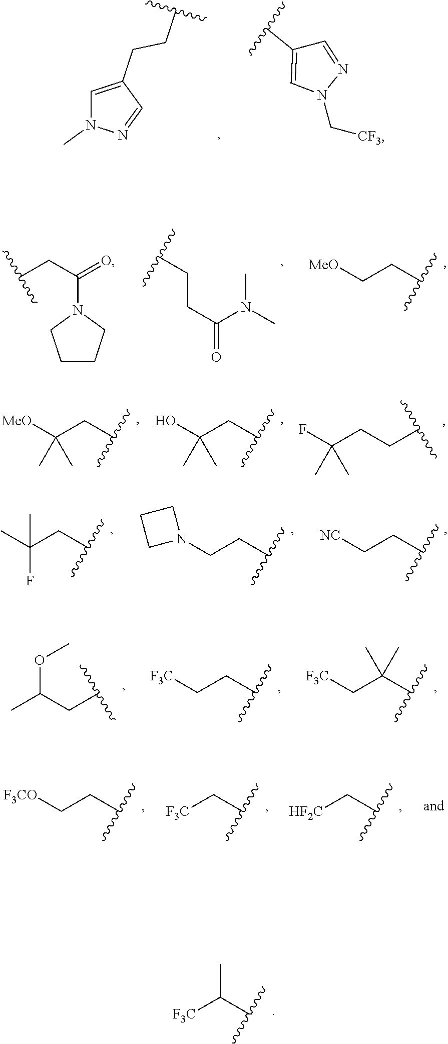

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

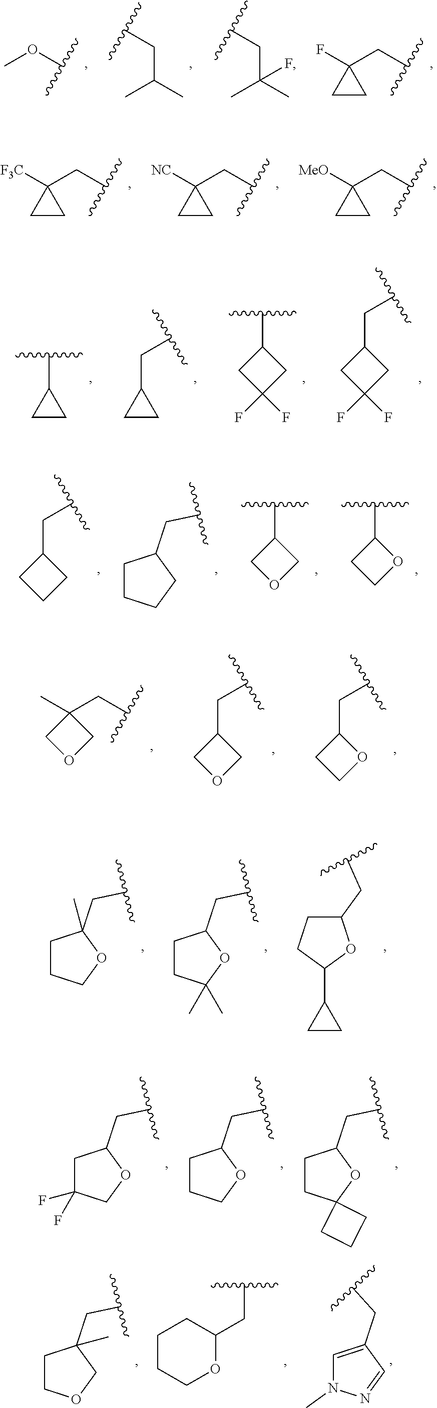

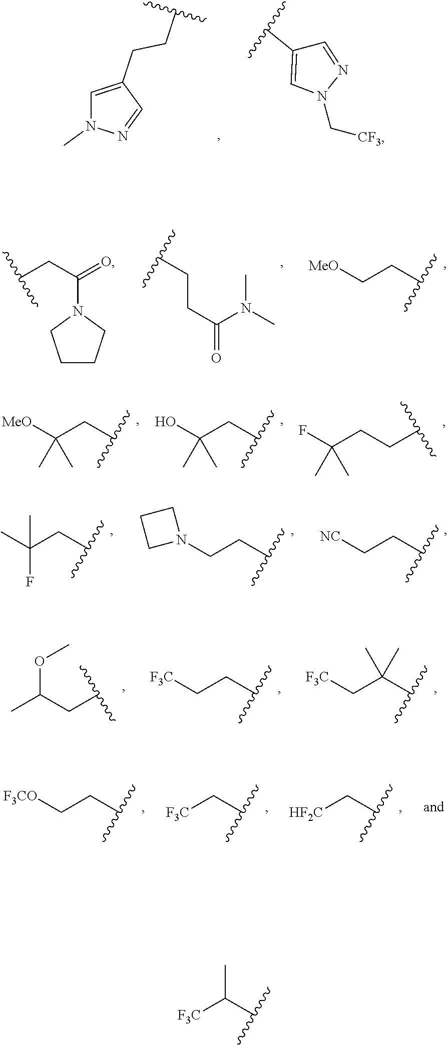

In some embodiments, R² and R³ together with the nitrogen atom to which they are attached form an optionally substituted heterocyclyl selected from the group consisting of

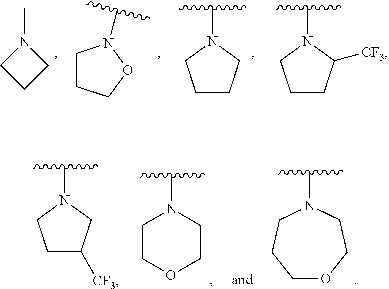

In some embodiments, R² and R³ together with the nitrogen atom to which they are attached form an optionally substituted heterocyclyl selected from the group consisting of

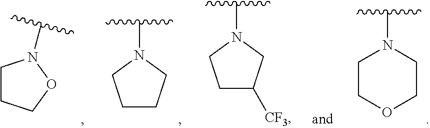 [SMILES: *N1CCCO1 ; *N1CCCC1 ; *N1CCC(C(F)(F)F)C1 ; *N1CCOCC1]

In some embodiments, E is selected from the group consisting of

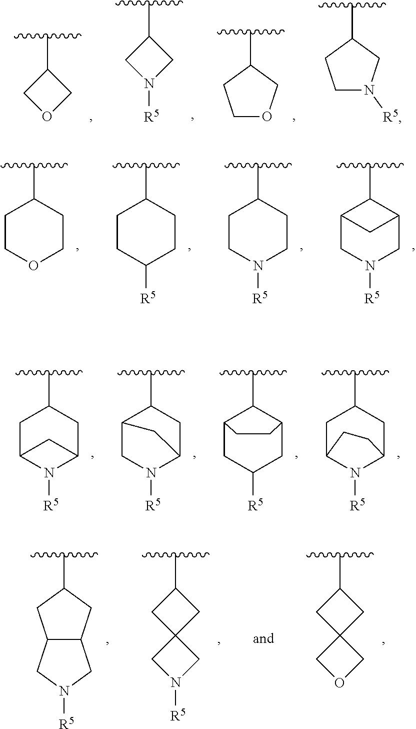

wherein R⁵ is independently H, alkyl, cycloalkyl, haloalkyl, or heterocyclyl.

In some embodiments, E is selected from the group consisting of

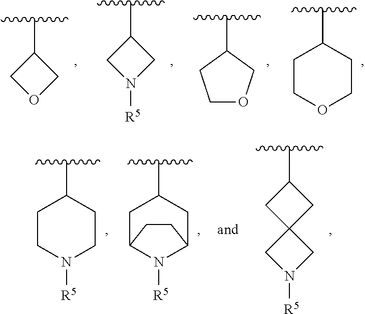

wherein R⁵ is independently H, alkyl, cycloalkyl, haloalkyl, or heterocyclyl.

In some embodiments, E is selected from the group consisting of

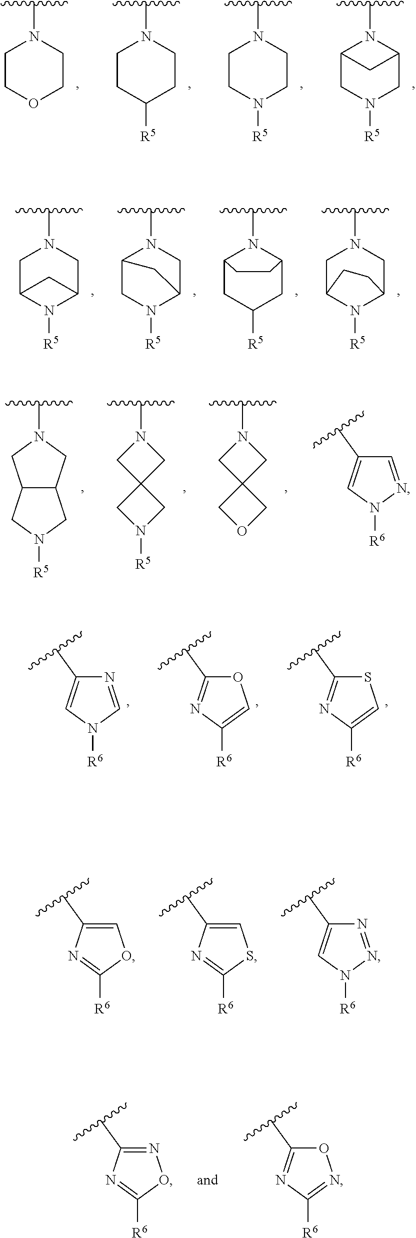

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

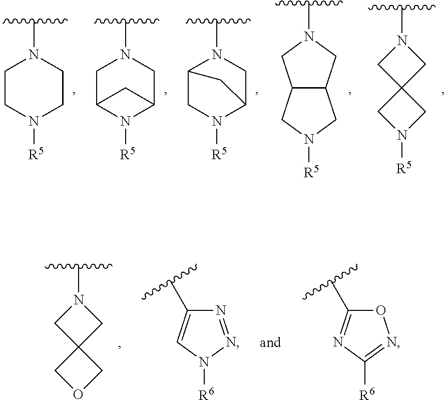

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

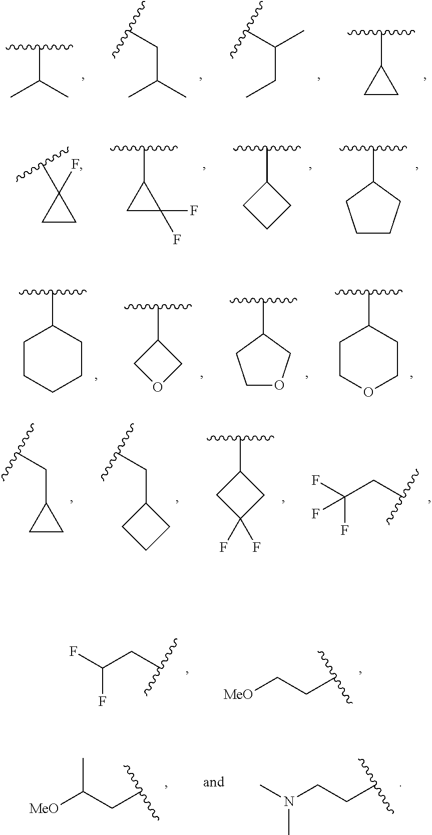

In some embodiments, E is selected from the group consisting of H, methyl, ethyl, trifluoromethyl, difluoromethyl,

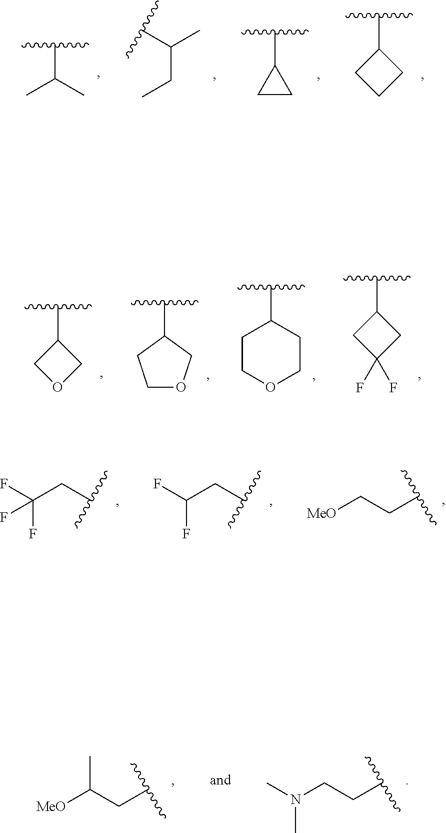

In one embodiment, described herein is a compound represented by Formula XIII:

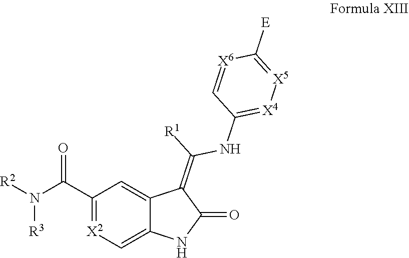

or a pharmaceutically acceptable salt, enantiomer, stereoisomer, or tautomer thereof, wherein:
X² is selected from the group consisting of N, CH, CF, and C-Cl;
X⁴, X⁵, and X⁶ are each independently selected from the group consisting of N, CH, and CF;
   provided that not more than two of X⁴, X⁵, and X⁶ are N;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, X² is CH. In other embodiments, X² is CF, C-Cl, or N.

In some embodiments, R¹ is alkyl, for example, methyl or ethyl. In some embodiments, R¹ is methyl. In embodiments, R¹ is ethyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

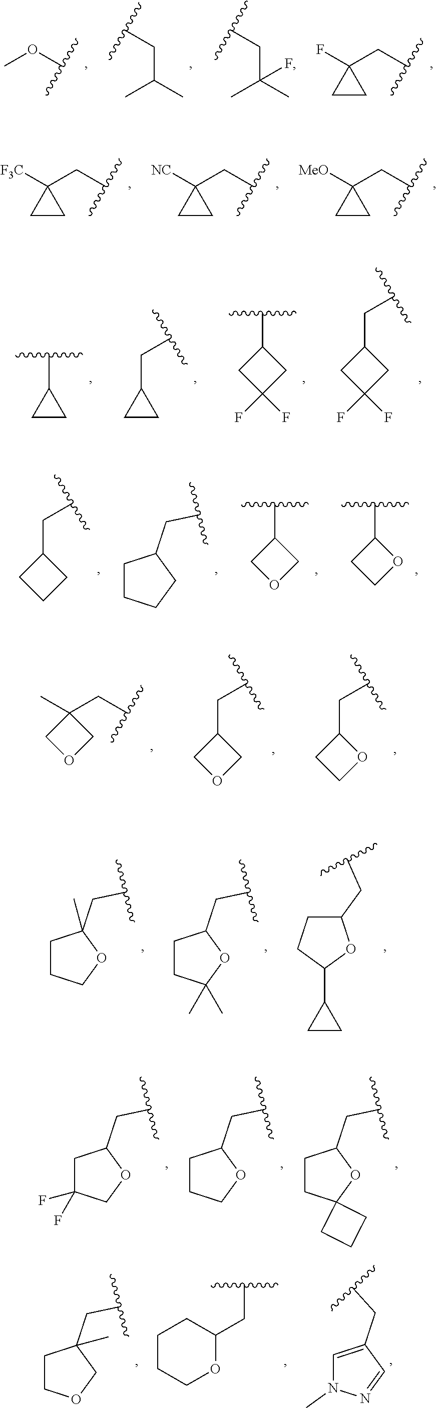

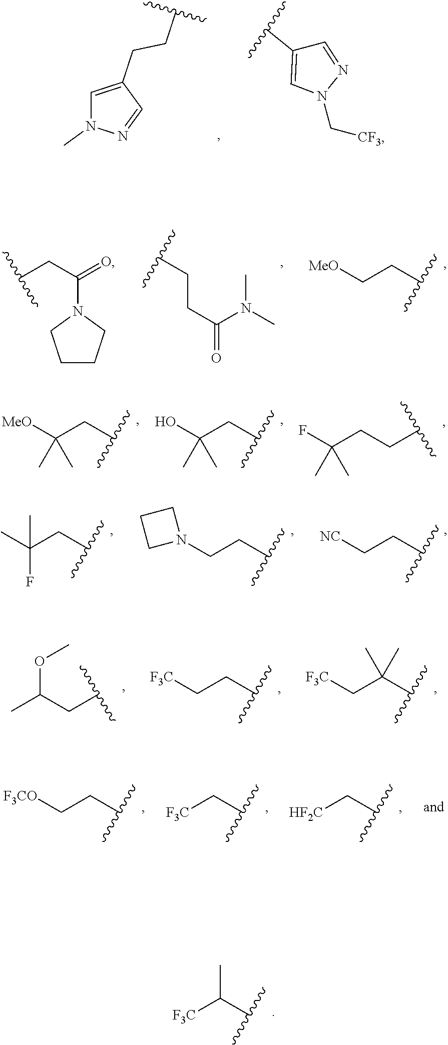

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

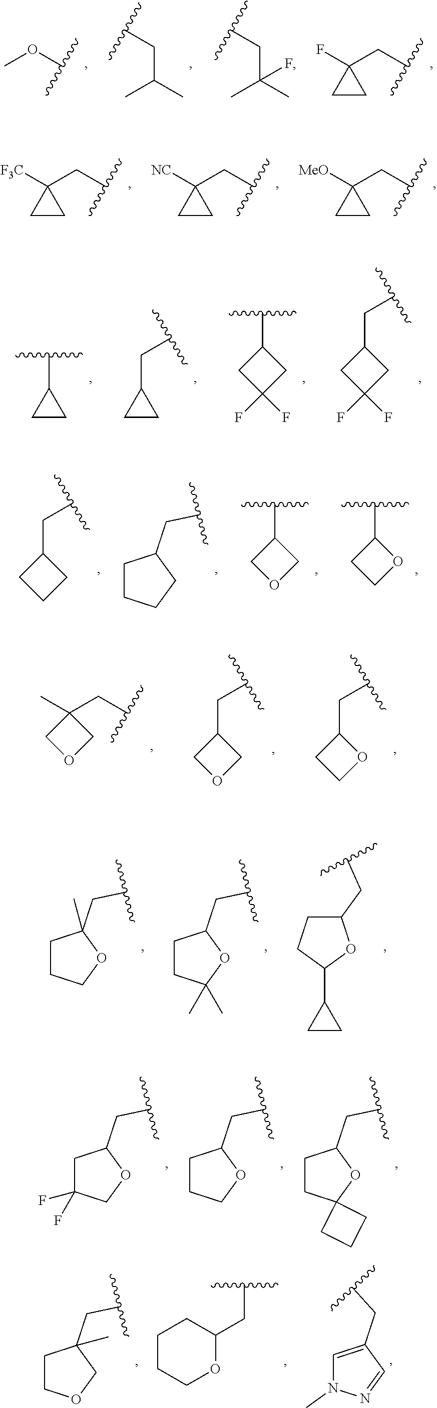

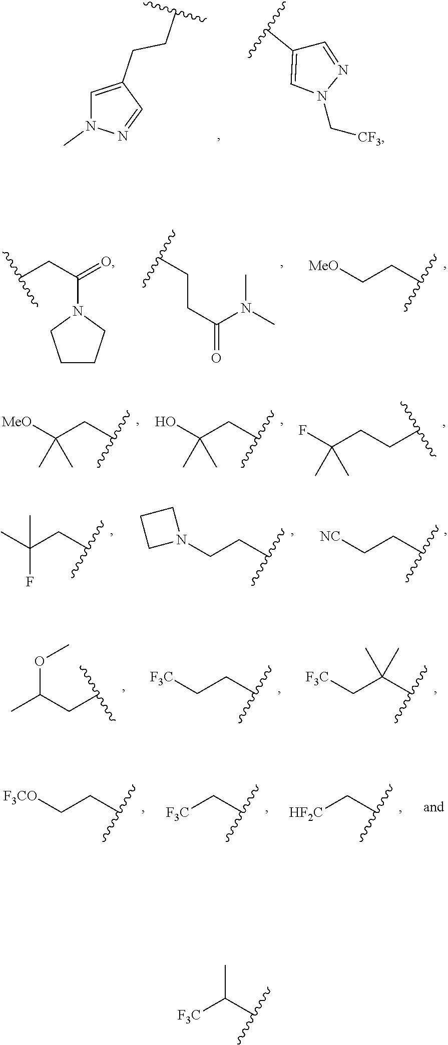

In some embodiments, R² and R³ together with the nitrogen atom to which they are attached form an optionally substituted heterocyclyl selected from the group consisting of

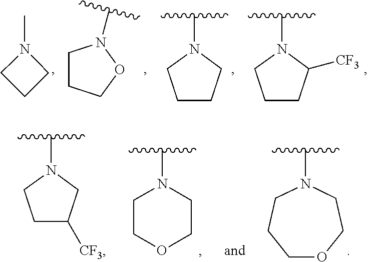

In some embodiments, R² and R³ together with the nitrogen atom to which they are attached form an optionally substituted heterocyclyl selected from the group consisting of

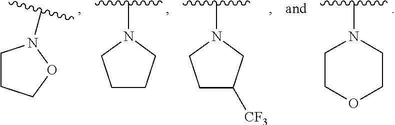

In some embodiments, E is selected from the group consisting of

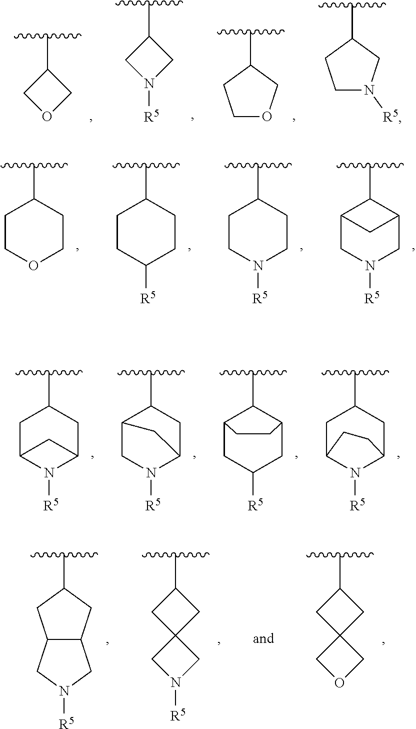

wherein R⁵ is independently H, alkyl, cycloalkyl, haloalkyl, or heterocyclyl.

In some embodiments, E is selected from the group consisting of

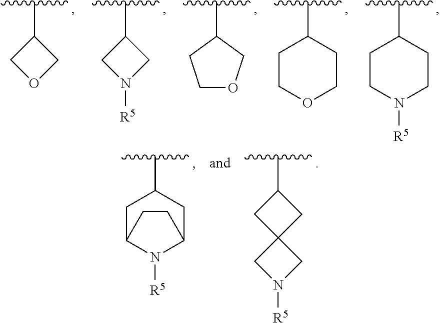

wherein R⁵ is independently H, alkyl, cycloalkyl, haloalkyl, or heterocyclyl.

In some embodiments, E is selected from the group consisting of

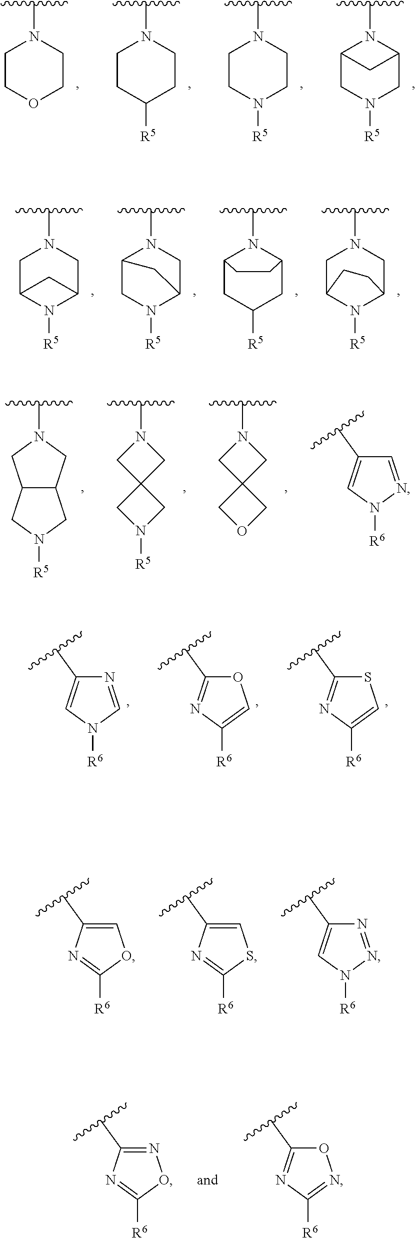

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

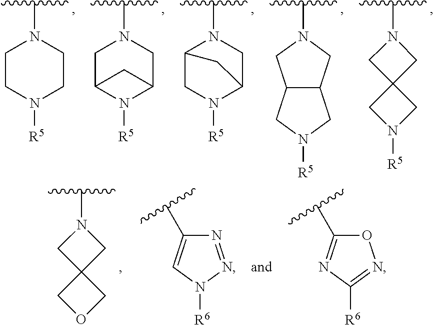

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

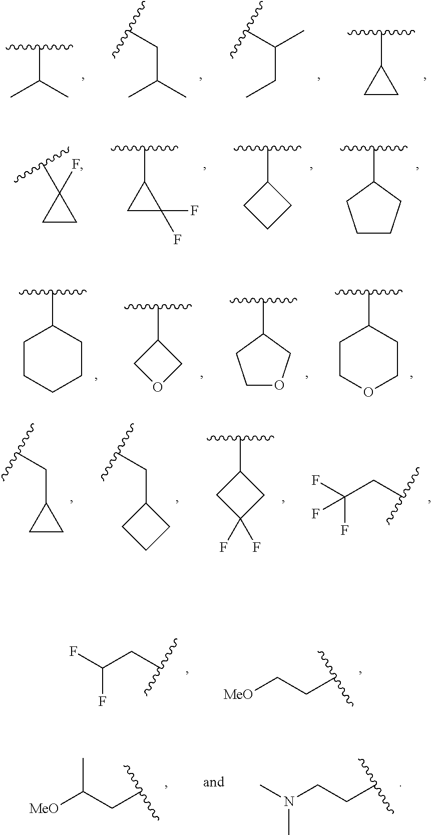

In some embodiments, E is selected from the group consisting of H, methyl, ethyl, trifluoromethyl, difluoromethyl,

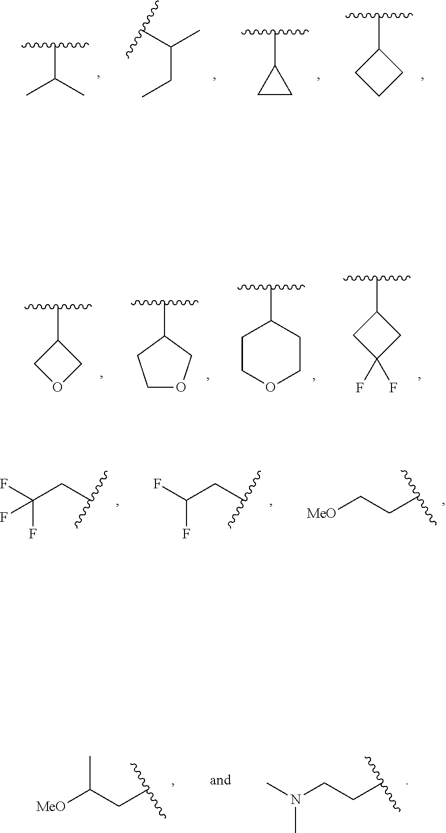

In one embodiment, described herein is a compound represented by Formula XIV:

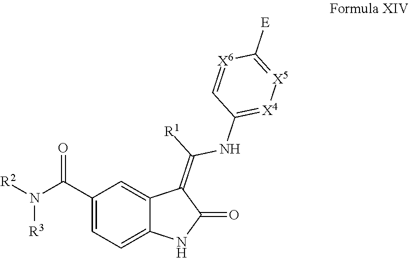

or a pharmaceutically acceptable salt, enantiomer, stereoisomer, or tautomer thereof, wherein:
X⁴, X⁵, and X⁶ are each independently selected from the group consisting of N, CH, and CF;
   provided that not more than two of X⁴, X⁵, and X⁶ are N;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

In some embodiments, R¹ is alkyl, for example, methyl or ethyl. In some embodiments, R¹ is methyl. In embodiments, R¹ is ethyl.

In some embodiments, R² is H. In some embodiments, R² is H and R³ is alkoxy. In other embodiments, R² is H and R³ is haloalkyl. In some embodiments, R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R² is H and R³ is cycloalkyl optionally substituted with one or more alkyl, alkoxy, amine, amide, halogen, haloalkyl, haloalkoxy, hydroxy, or cyano. In other embodiments, R² is H and R³ is cycloalkyl optionally substituted with one or more halogen. In some embodiments, wherein R² is H and R³ is heteroaryl optionally substituted with one or more alkyl, alkoxy, amine, amide, halogen, haloalkyl, haloalkoxy, hydroxy, or cyano. In some embodiments, R² is H and R³ is heteroaryl optionally substituted with haloalkyl. In other embodiments, R² is H and R³ is heterocyclyl optionally substituted with one or more alkyl, alkoxy, amine, amide, halogen, haloalkyl, haloalkoxy, hydroxy, or cyano. In some embodiments, R² is H and R³ is heterocyclyl. In some embodiments, R² is H and R³ is selected from the group consisting of methyl, ethyl, n-propyl, isopropyl,

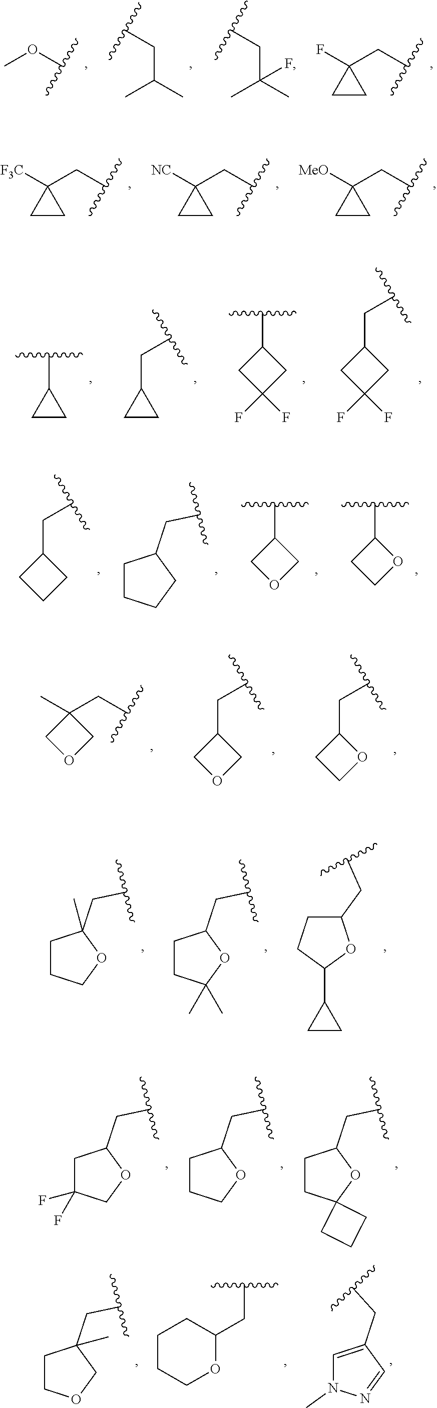

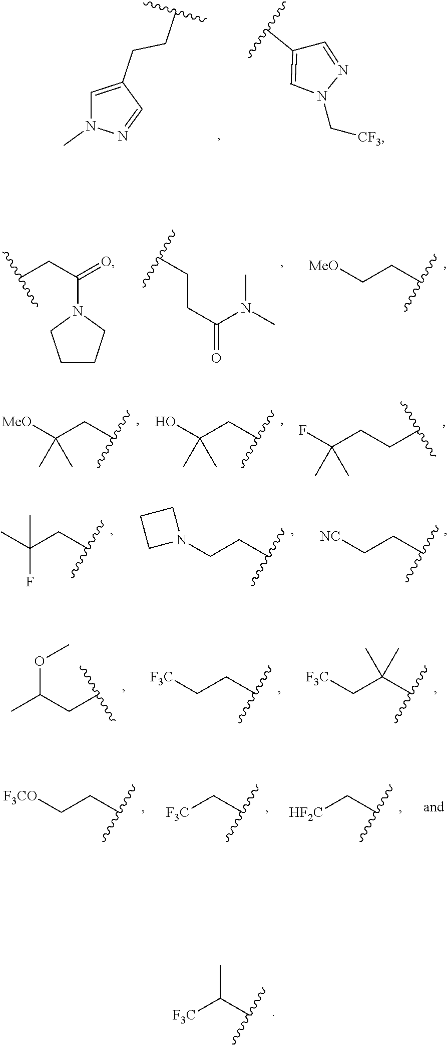

In some embodiments, R² is alkyl, for example, methyl. In some embodiments, R² is cycloalkyl, for example, cyclopropyl.

In some embodiments, R³ is alkoxy. In other embodiments, R³ is haloalkyl. In some embodiments, R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl. In some embodiments, R³ is cycloalkyl optionally substituted with one or more alkyl, alkoxy, amine, amide, halogen, haloalkyl, haloalkoxy, hydroxy, or cyano. In some embodiments, R³ is cycloalkyl optionally substituted with one or more halogen. In other embodiments, R³ is heteroaryl optionally substituted with one or more alkyl, alkoxy, amine, amide, halogen, haloalkyl, haloalkoxy, hydroxy, or cyano. In some embodiments, R³ is heteroaryl optionally substituted with haloalkyl. In some embodiments, R³ is heterocyclyl optionally substituted with one or more alkyl, alkoxy, amine, amide, halogen, haloalkyl, haloalkoxy, hydroxy, or cyano. In some embodiments, R³ is heterocyclyl. In some embodiments, R³ is selected from the group consisting of methyl, ethyl, n-propyl, isopropyl,

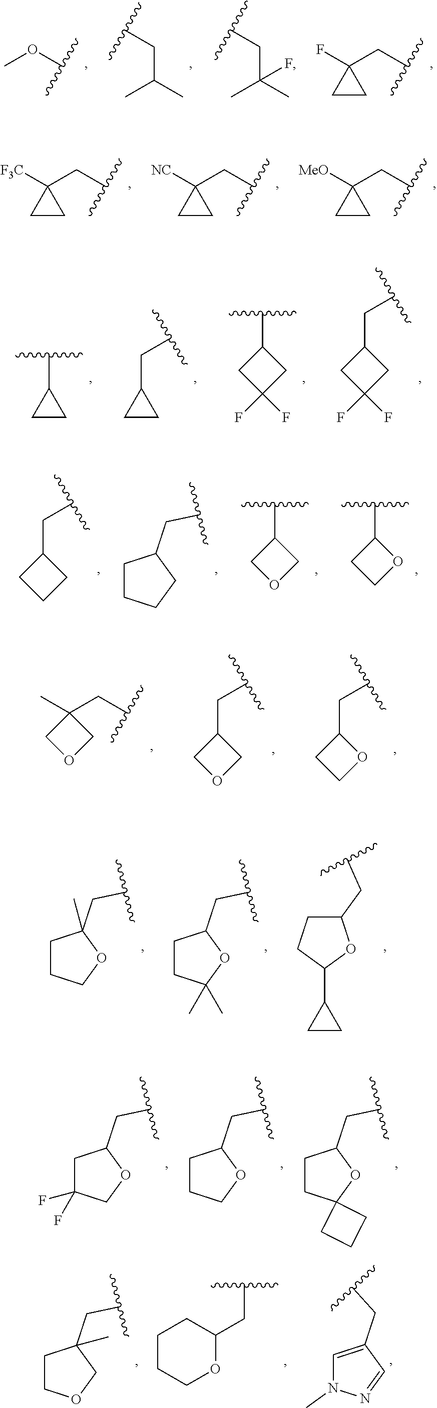

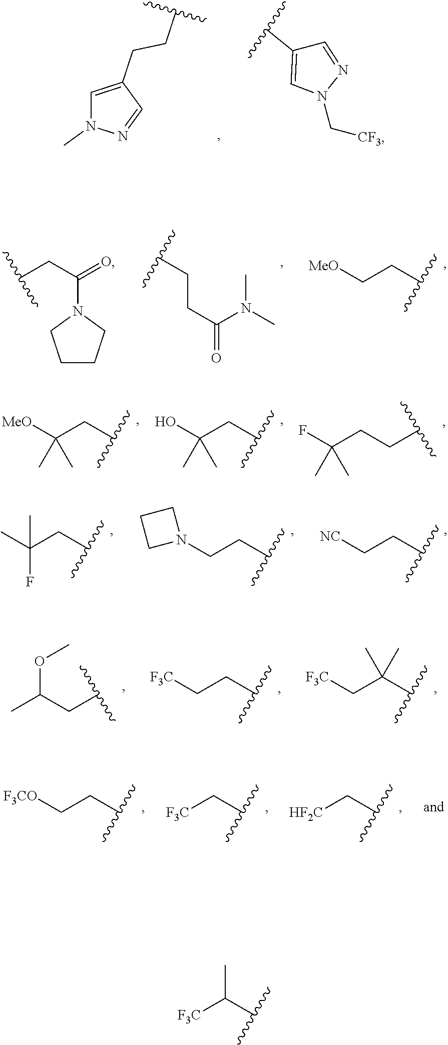

In some embodiments, R² and R³ together with the nitrogen atom to which they are attached form an optionally substituted heterocyclyl selected from the group consisting of

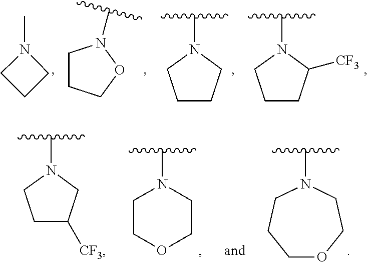

In some embodiments, R² and R³ together with the nitrogen atom to which they are attached form an optionally substituted heterocyclyl selected from the group consisting of

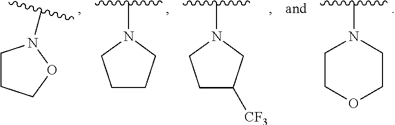

In some embodiments, E is selected from the group consisting of

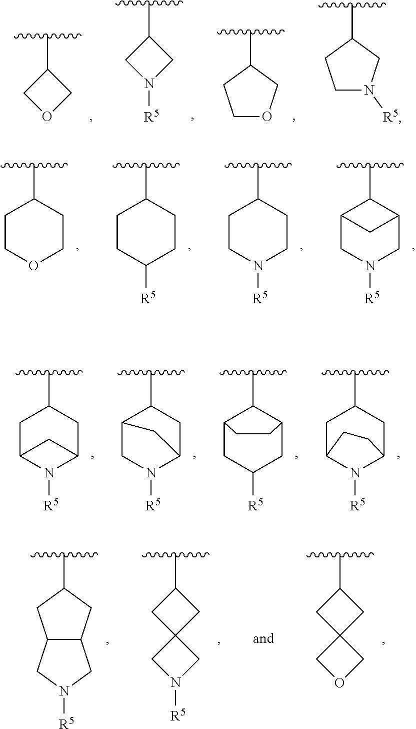

wherein R⁵ is independently H, alkyl, cycloalkyl, haloalkyl, or heterocyclyl.

In some embodiments, E is selected from the group consisting of

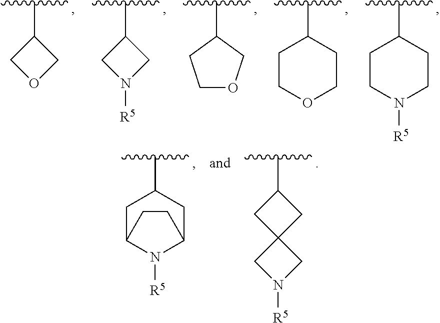

wherein R⁵ is independently H, alkyl, cycloalkyl, haloalkyl, or heterocyclyl.

In some embodiments, E is selected from the group consisting of

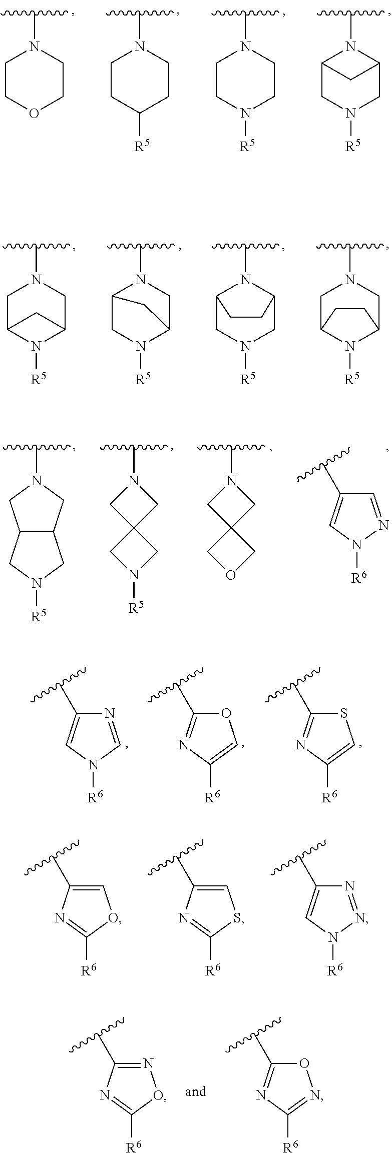

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

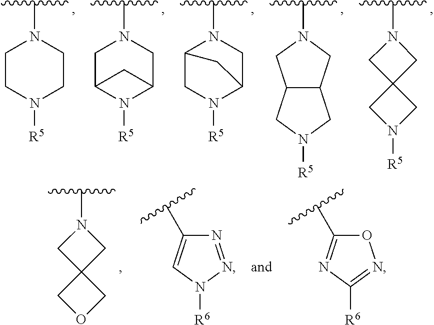

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

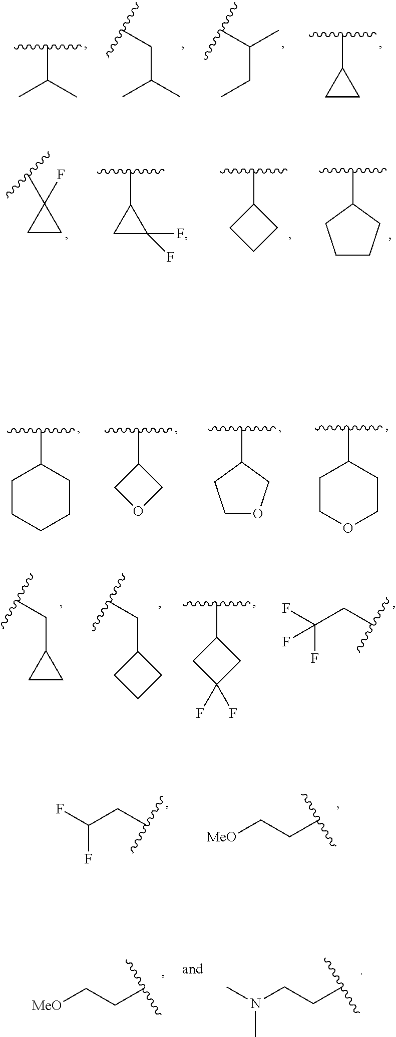

In some embodiments, E is selected from the group consisting of H, methyl, ethyl, trifluoromethyl, difluoromethyl,

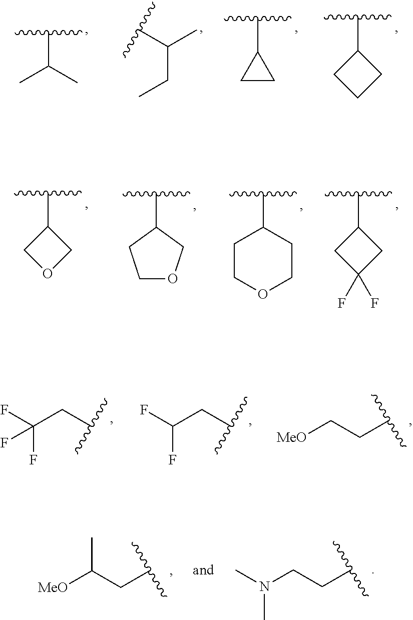

In another embodiment, described herein is a compound represented by Formula XV:

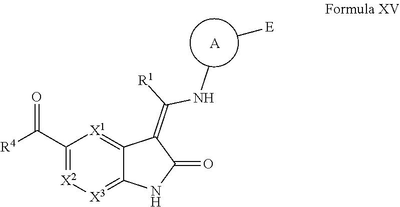

or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
   provided that not more than two of X¹, X², and X³ are N;
A is selected from the group consisting of
   optionally substituted heteroaryl wherein the optionally substituted substituent, at each occurrence, is independently selected from the group consisting of alkyl, alkoxy, halogen, haloalkyl, haloalkoxy, hydroxy, and cyano, and
   optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R⁴ is selected from the group consisting of
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano; provided that
      when X¹, X², and X³ are CH, R⁴ is C₁₋₅ alkyl optionally substituted by one or more fluorine atoms, and A is an optionally substituted phenyl, E is not alkyl substituted with amine.

In some embodiments, X¹ is CH. In other embodiments, X¹ is CF or N. In some embodiments, X³ is CH. In other embodiments, X³ is CF or N. In some embodiments, X² is CH. In other embodiments, X² is CF, C-Cl, or N. In some embodiments, X¹, X², and X³ are CH.

In some embodiments, R¹ is alkyl, for example, methyl or ethyl. In some embodiments, R¹ is methyl. In embodiments, R¹ is ethyl.

In some embodiments, A is selected from the group consisting of

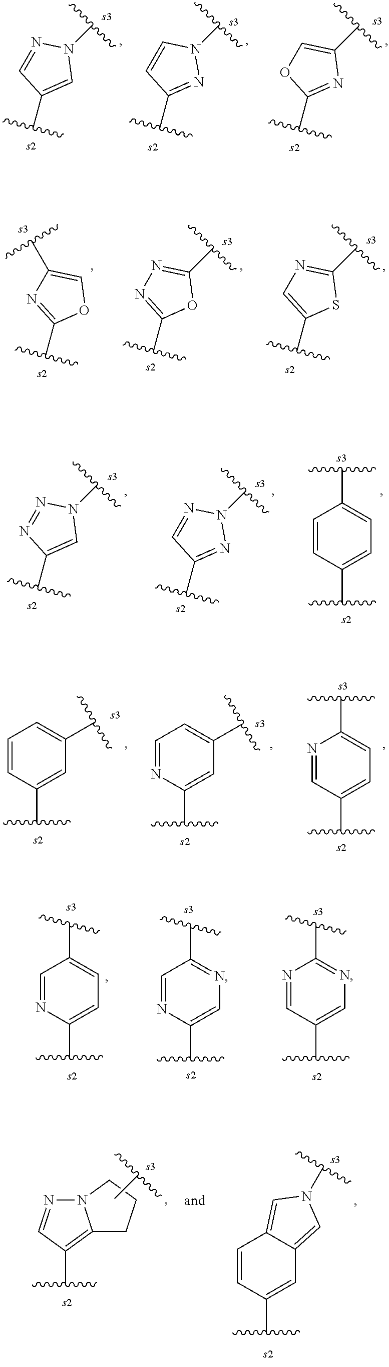

each of which is optionally substituted with alkyl, alkoxy, halogen, haloalkyl, haloalkoxy, hydroxy, or cyano, wherein s2 is the site covalently linked to —NH— and s3 is the site covalently linked to E.

In some embodiments, A is phenyl optionally substituted with halogen or alkyl.

In another embodiment, A is 5-6 membered heteroaryl optionally substituted with halogen or alkyl. In some embodiments, A is pyrazole optionally substituted with halogen or alkyl.

In some embodiments, R⁴ is selected from the group consisting of optionally substituted alkyl and cycloalkyl. In some embodiments, R⁴ is selected from the group consisting of methyl,

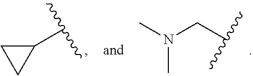

In some embodiments, E is selected from the group consisting of

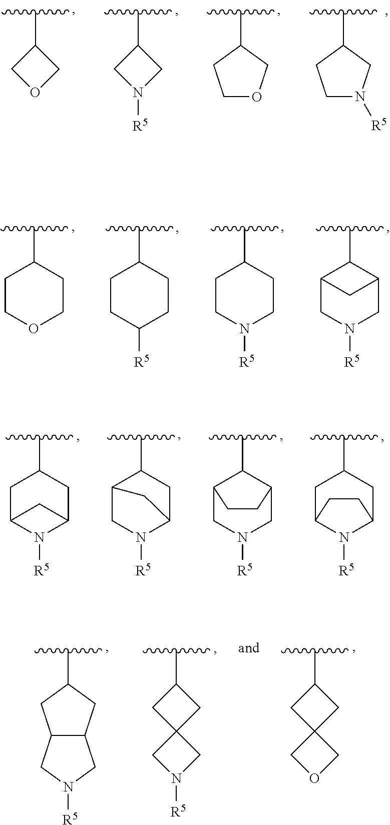

wherein R⁵ is independently H, alkyl, cycloalkyl, haloalkyl, or heterocyclyl.

In some embodiments, E is selected from the group consisting of

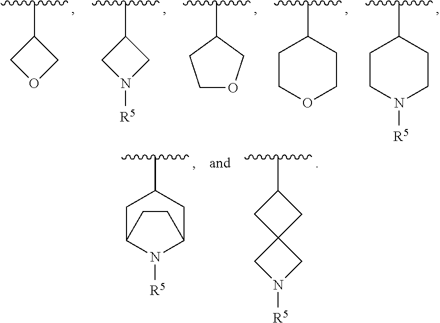

wherein R⁵ is independently H, alkyl, cycloalkyl, haloalkyl, or heterocyclyl.

In some embodiments, when A is pyrazole, E is selected from the group consisting of

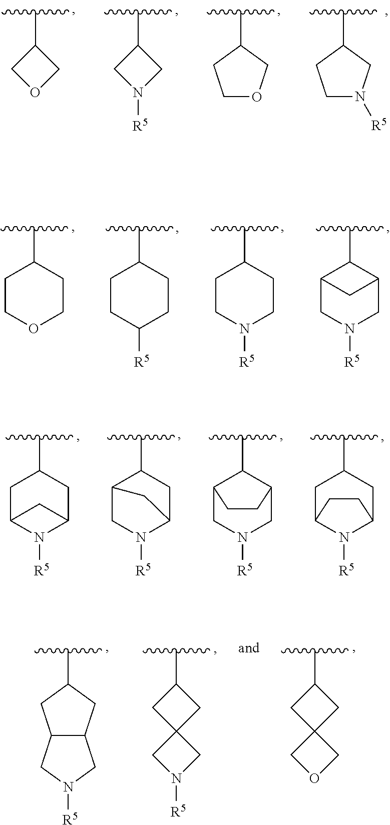

wherein R⁵ is independently H, alkyl, cycloalkyl, haloalkyl, or heterocyclyl.

In some embodiments, when A is pyrazole, E is selected from the group consisting of

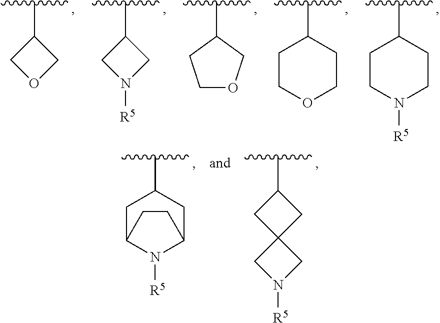

wherein R⁵ is independently H, alkyl, cycloalkyl, haloalkyl, or heterocyclyl.

In some embodiments, E is selected from the group consisting of

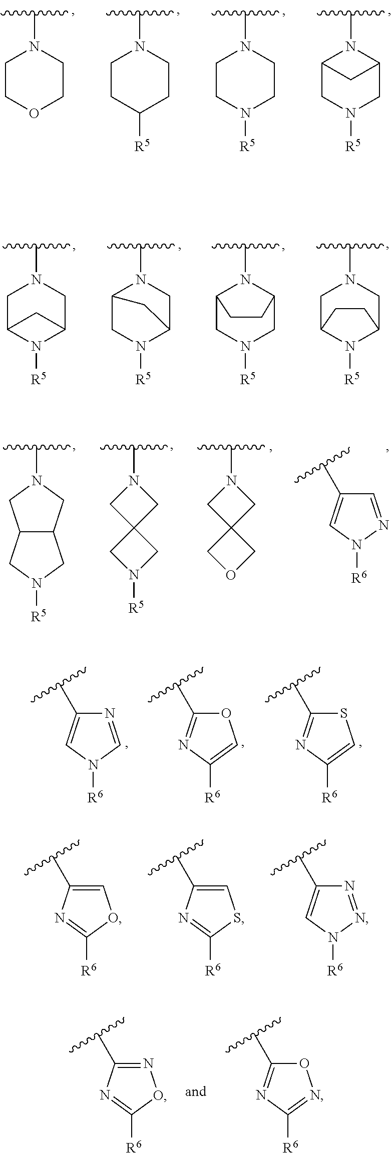

wherein R⁵ is independently H, alkyl, cycloalkyl, haloalkyl, or heterocyclyl, and R is independently H, alkyl, cycloalkyl, or haloalkyl.

In some embodiments, E is selected from the group consisting of

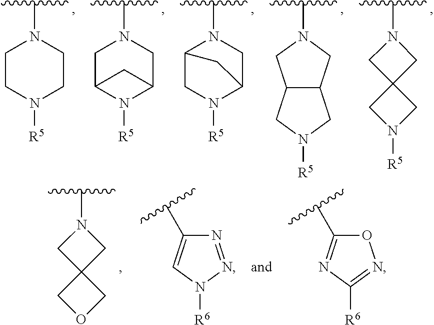

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

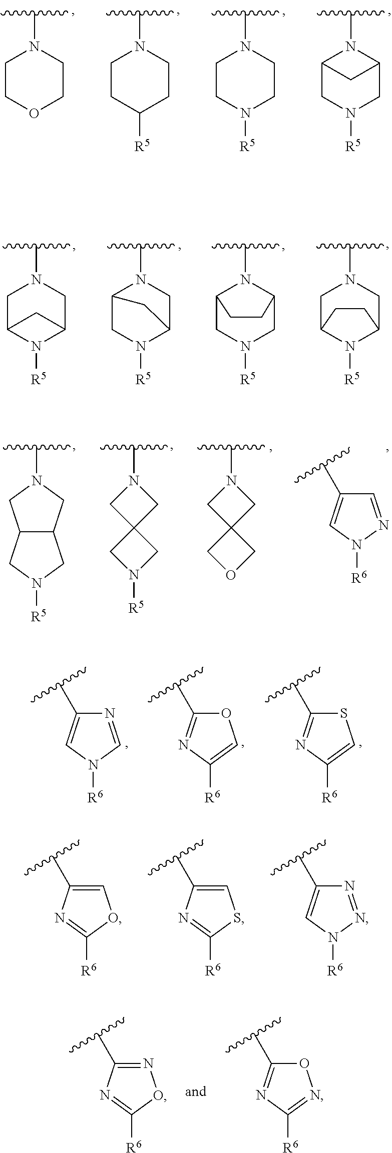

wherein R⁵ is independently H, alkyl, cycloalkyl, haloalkyl, or heterocyclyl, and R⁶ is independently H, alkyl, cycloalkyl, or haloalkyl.

In some embodiments, when A is phenyl or 6-membered heteroaryl, E is selected from the group consisting of

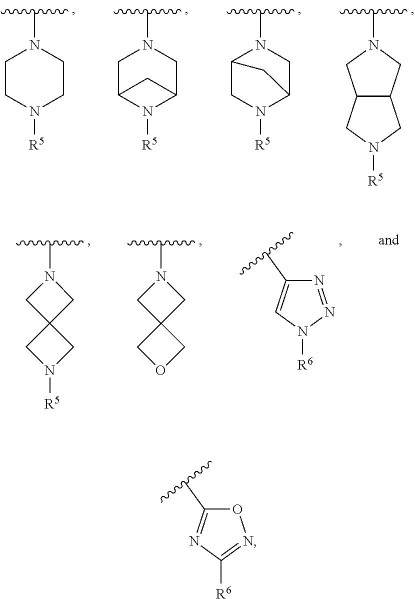

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

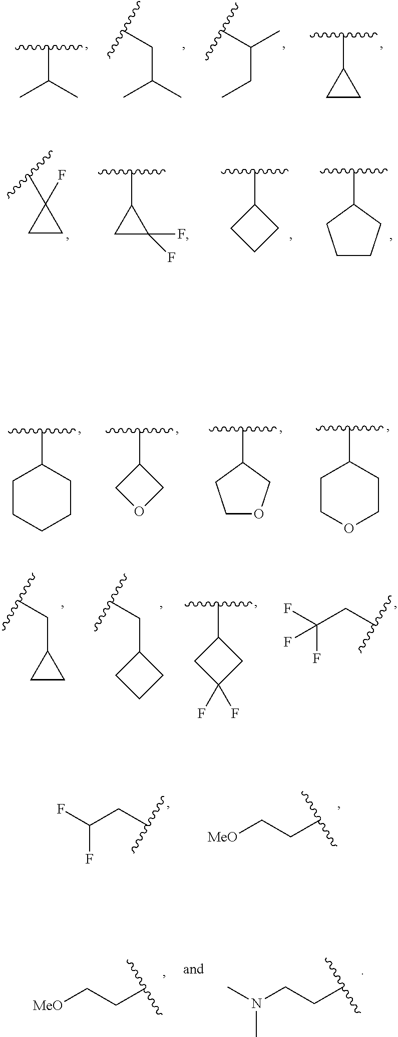

In some embodiments, E is selected from the group consisting of H, methyl, ethyl, trifluoromethyl, difluoromethyl,

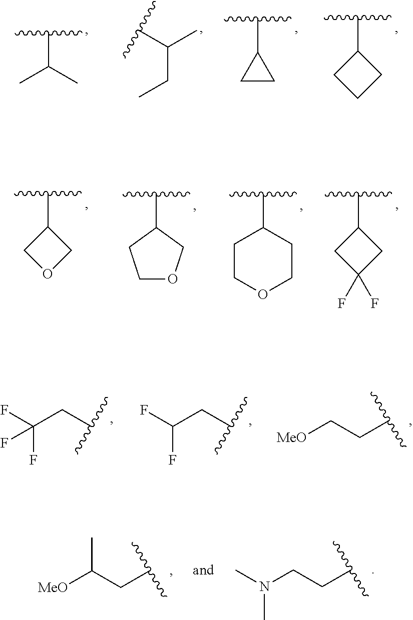

In one embodiment, described herein is a compound represented by Formula XVI:

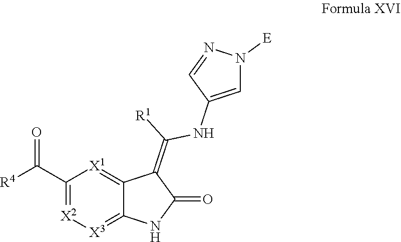

or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
   provided that not more than two of X¹, X², and X³ are N;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R⁴ is selected from the group consisting of
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
      optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano.

In some embodiments, X¹ is CH. In other embodiments, X¹ is CF or N. In some embodiments, X³ is CH. In other embodiments, X³ is CF or N. In some embodiments, X² is CH. In other embodiments, X² is CF, C-Cl, or N. In some embodiments, X¹, X², and X³ are CH.

In some embodiments, R¹ is alkyl, for example, methyl or ethyl. In some embodiments, R¹ is methyl. In embodiments, R¹ is ethyl.

In some embodiments, R⁴ is selected from the group consisting of optionally substituted alkyl and cycloalkyl. In some embodiments, R⁴ is selected from the group consisting and of methyl,

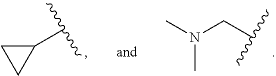

In some embodiments, E is selected from the group consisting of

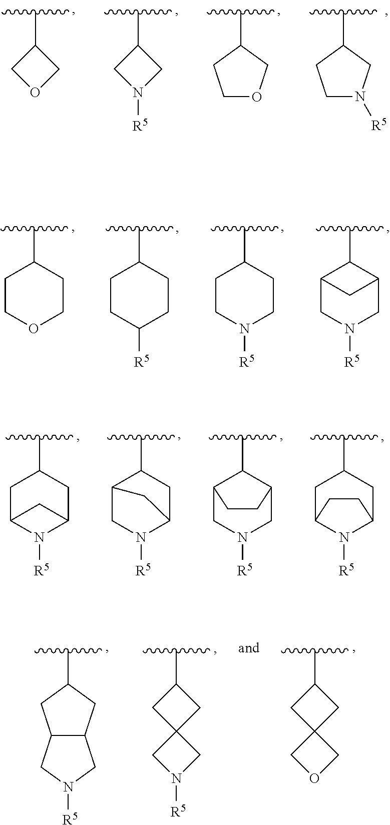

wherein R⁵ is independently H, alkyl, cycloalkyl, haloalkyl, or heterocyclyl.

In some embodiments, E is selected from the group consisting of

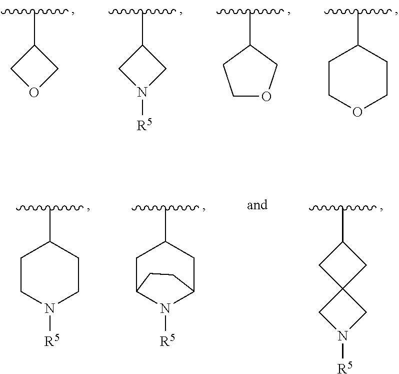

wherein R⁵ is independently H, alkyl, cycloalkyl, haloalkyl, or heterocyclyl.

In some embodiments, E is selected from the group consisting of

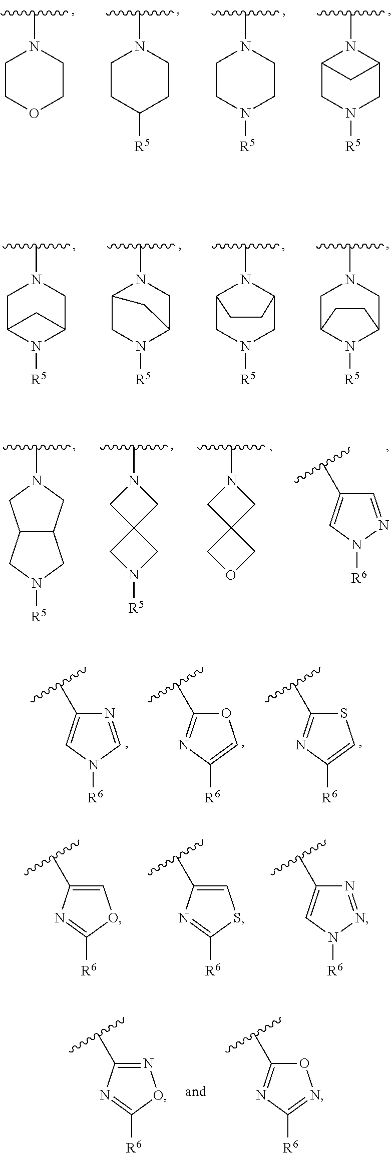

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

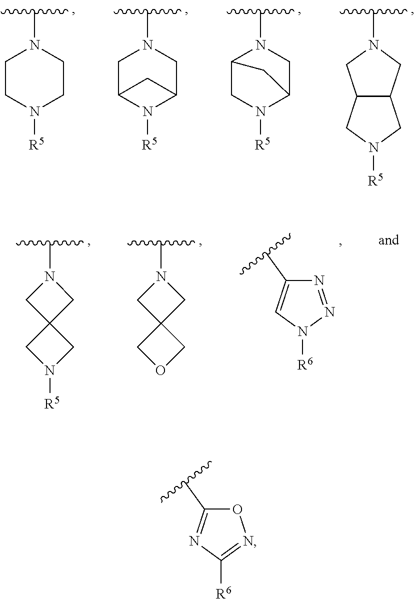

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

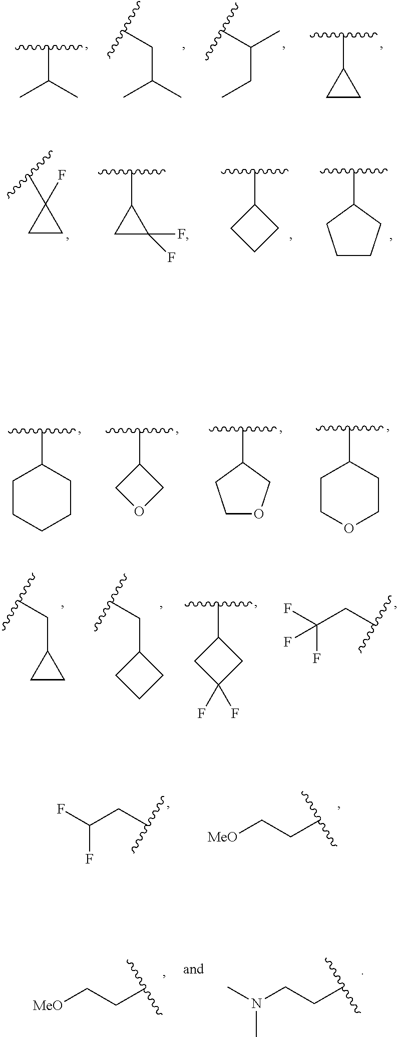

In some embodiments, E is selected from the group consisting of H, methyl, ethyl, trifluoromethyl, difluoromethyl,

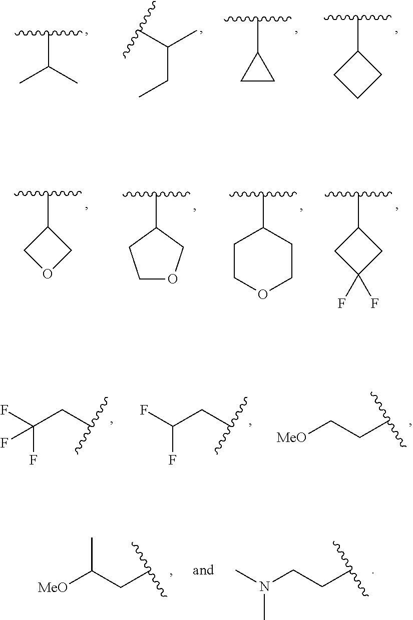

In another embodiment, described herein is a compound represented by Formula XVII:

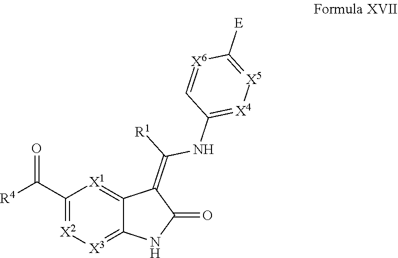

or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
   provided that not more than two of X¹, X², and X³ are N;
X⁴, X⁵, and X⁶ are each independently selected from the group consisting of N, CH, and CF;
   provided that not more than two of X⁴, X⁵, and X⁶ are N;
E is selected from the group consisting of H, haloalkyl,
   optionally substituted alkyl wherein the optionally substituted substituent, at each occurrence, is independently selected from the group consisting of amine, alkoxy, halogen, haloalkyl, haloalkoxy, hydroxy, and cyano,
   optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
   optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
   optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R⁴ is selected from the group consisting of
   optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
   optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
   optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano;
      provided that when X¹, X², X³, X⁴, X⁵, and X⁶ are CH, and R⁴ is C₁₋₅ alkyl optionally substituted by one or more fluorine atoms, E is not alkyl substituted with amine.

In some embodiments, X¹ is CH. In other embodiments, X¹ is CF or N. In some embodiments, X³ is CH. In other embodiments, X³ is CF or N. In some embodiments, X² is CH. In other embodiments, X² is CF, C-Cl, or N. In some embodiments, X¹, X², and X³ are CH.

In some embodiments, R¹ is alkyl, for example, methyl or ethyl. In some embodiments, R¹ is methyl. In embodiments, R¹ is ethyl.

In some embodiments, R⁴ is selected from the group consisting of optionally substituted alkyl and cycloalkyl. In some embodiments, R⁴ is selected from the group consisting and of methyl,

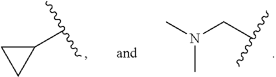

In some embodiments, E is select from the group consisting of

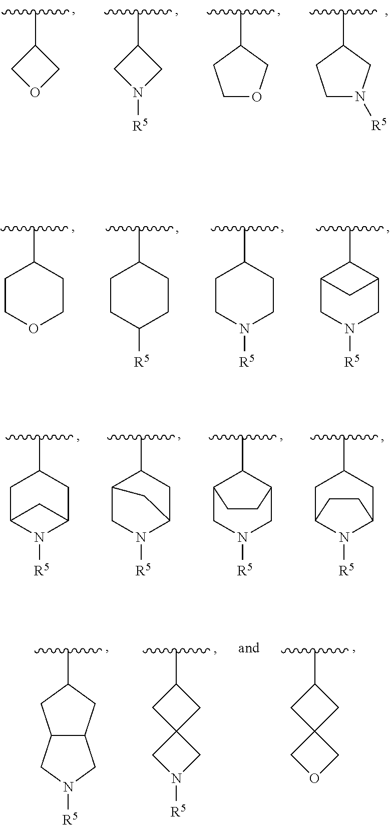

wherein R⁵ is independently H, alkyl, cycloalkyl, haloalkyl, or heterocyclyl.

In some embodiments, E is selected from the group consisting of

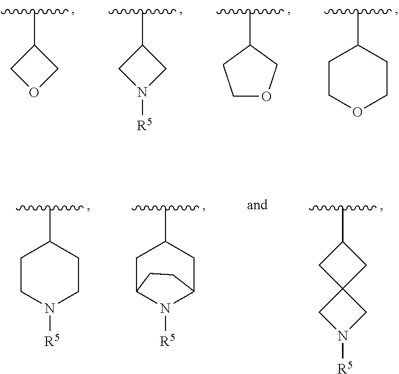

wherein R⁵ is independently H, alkyl, cycloalkyl, haloalkyl, or heterocyclyl.

In some embodiments, E is selected from the group consisting of

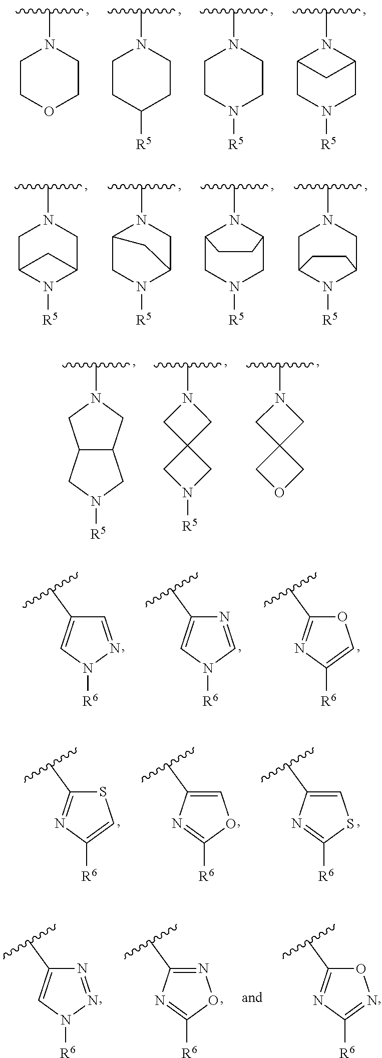

wherein R⁵ is independently H, alkyl, cycloalkyl, haloalkyl, or heterocyclyl, and R⁶ is independently H, alkyl, cycloalkyl, or haloalkyl.

In some embodiments, E is selected from the group consisting of

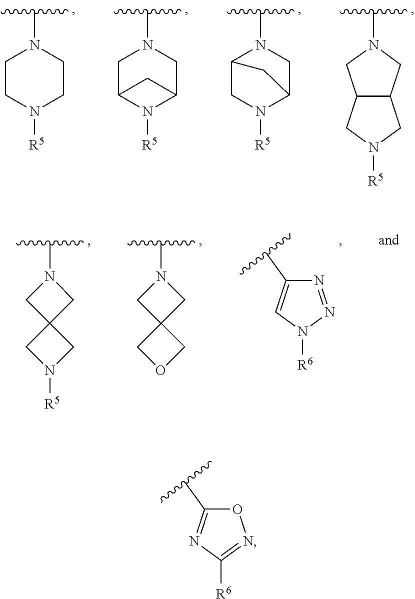

wherein R⁵ is independently H, alkyl, cycloalkyl, haloalkyl, or heterocyclyl, and R⁶ is independently H, alkyl, cycloalkyl, or haloalkyl.

In some embodiments, E is H. In other embodiments, E is alkyl optionally substituted with cycloalkyl, amine, or alkoxy. In some embodiments, E is alkyl. In some embodiments, E is haloalkyl. In some embodiments, E is cycloalkyl optionally substituted with one or more halogen. In some embodiments, E is 4-6 membered heterocyclyl. In some embodiments, E is selected from the group consisting of H, alkyl optionally substituted with cycloalkyl, amine, or alkoxy, haloalkyl, cycloalkyl optionally substituted with one or more halogen, and heterocyclyl.

In some embodiments, E is selected from the group consisting of H, methyl, ethyl, trifluoromethyl, difluoromethyl,

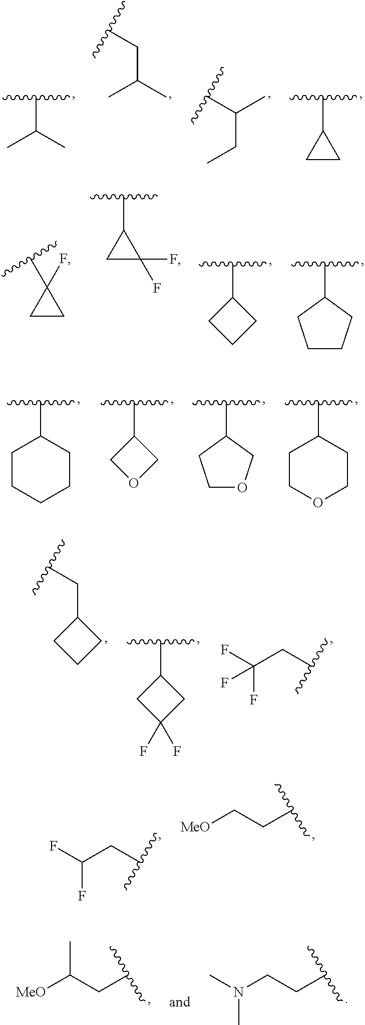

In some embodiments, E is selected from the group consisting of H, methyl, ethyl, trifluoromethyl, difluoromethyl,

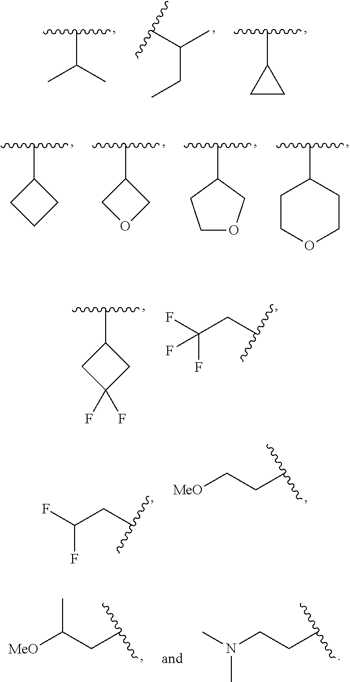

In an embodiment, described herein is a compound selected from the group consisting of

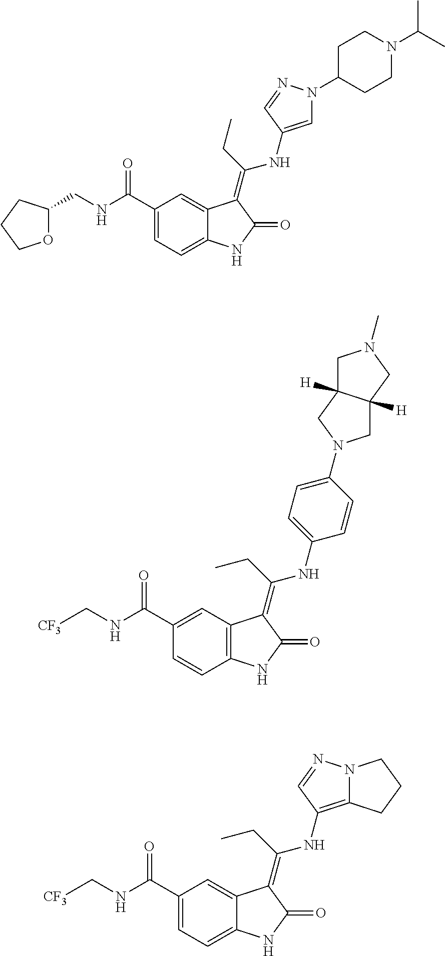 [SMILES: CC/C(Nc1cnn(C2CCN(C(C)C)CC2)c1)=C1/C(=O)Nc2ccc(C(=O)NC[C@H]3CCCO3)cc21 ; CC/C(Nc1ccc(N2C[C@H]3CN(C)C[C@H]3C2)cc1)=C1/C(=O)Nc2ccc(C(=O)NCC(F)(F)F)cc21 ; CC/C(Nc1cnn2c1CCC2)=C1/C(=O)Nc2ccc(C(=O)NCC(F)(F)F)cc21]

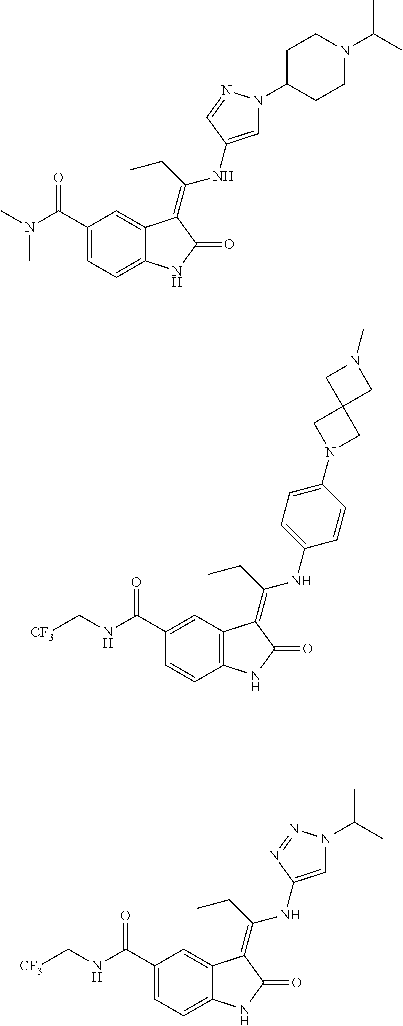 [SMILES: CC/C(Nc1cnn(C2CCN(C(C)C)CC2)c1)=C1/C(=O)Nc2ccc(C(=O)N(C)C)cc21 ; CC/C(Nc1ccc(N2CC3(CN(C)C3)C2)cc1)=C1/C(=O)Nc2ccc(C(=O)NCC(F)(F)F)cc21 ; CC/C(Nc1cn(C(C)C)nn1)=C1/C(=O)Nc2ccc(C(=O)NCC(F)(F)F)cc21]

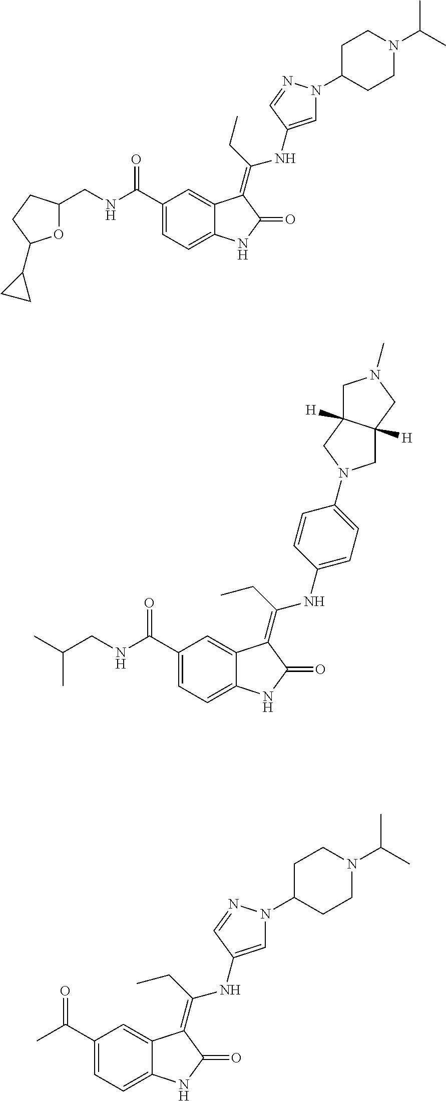

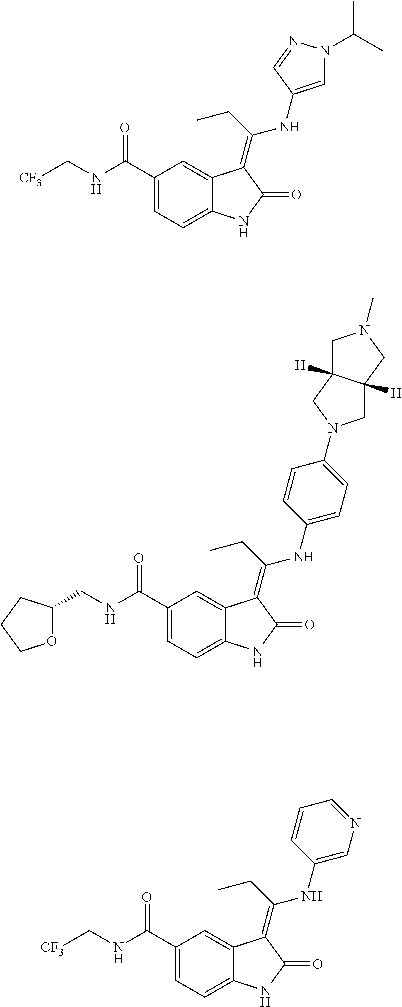 [SMILES: CC/C(Nc1cnn(C(C)C)c1)=C1/C(=O)Nc2ccc(C(=O)NCC(F)(F)F)cc21 ; CC/C(Nc1ccc(N2C[C@H]3CN(C)C[C@H]3C2)cc1)=C1/C(=O)Nc2ccc(C(=O)NC[C@H]3CCCO3)cc21 ; CC/C(Nc1cccnc1)=C1/C(=O)Nc2ccc(C(=O)NCC(F)(F)F)cc21]

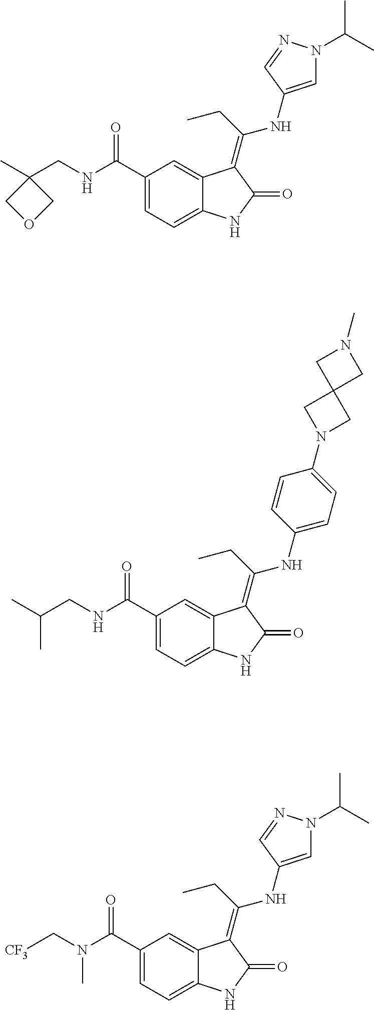

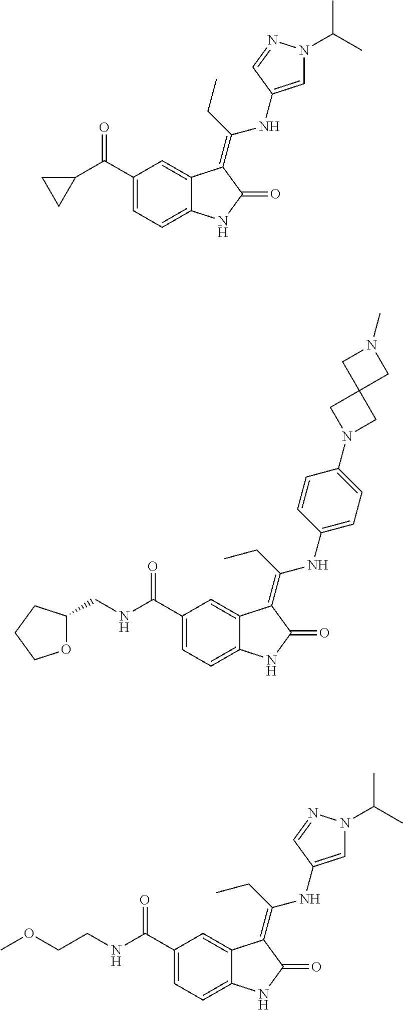

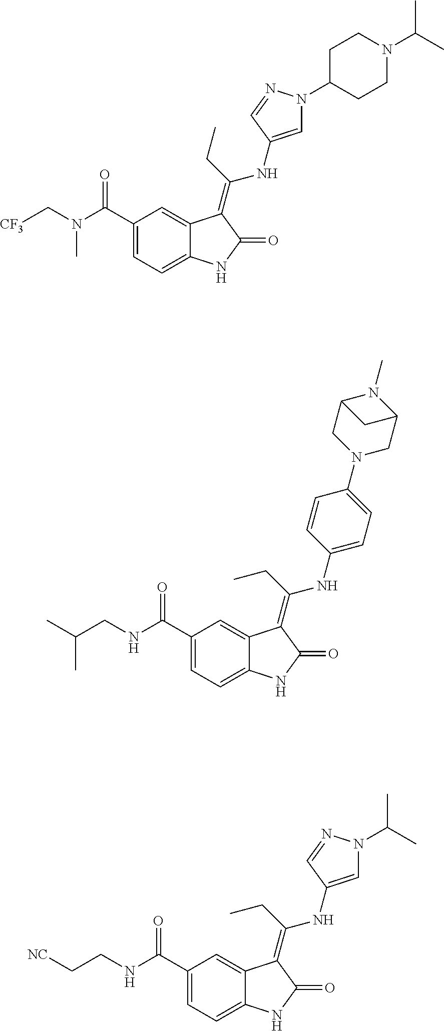

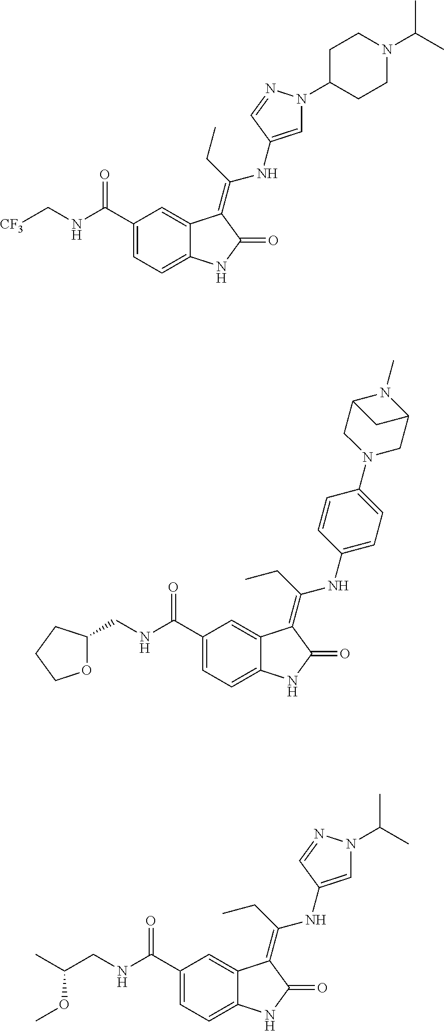

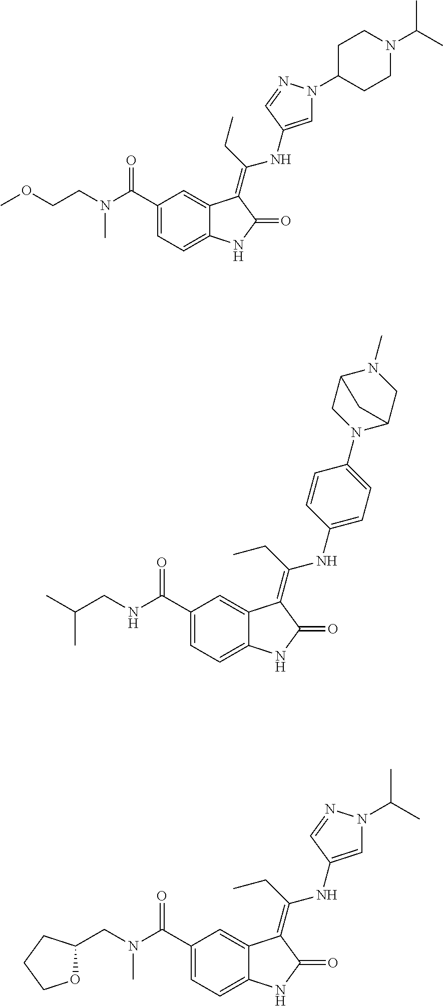

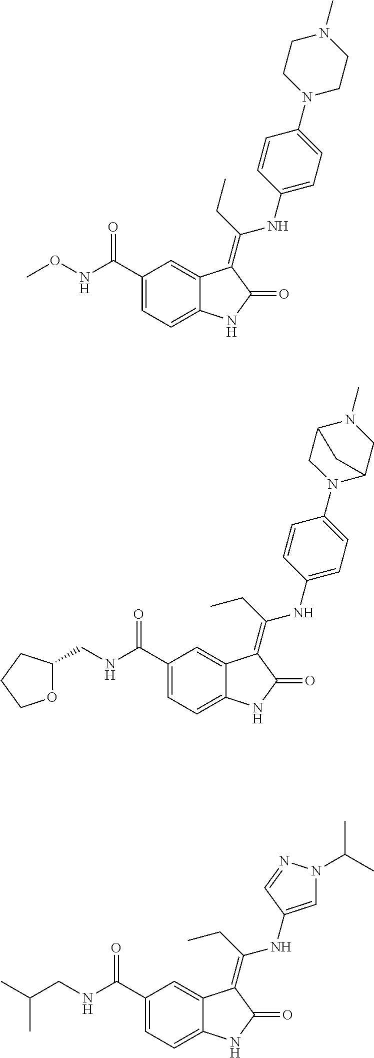 [SMILES: CC/C(Nc1ccc(N2CCN(C)CC2)cc1)=C1/C(=O)Nc2ccc(C(=O)NOC)cc21 ; CC/C(Nc1ccc(N2CC3CC2CN3C)cc1)=C1/C(=O)Nc2ccc(C(=O)NC[C@H]3CCCO3)cc21 ; CC/C(Nc1cnn(C(C)C)c1)=C1/C(=O)Nc2ccc(C(=O)NCC(C)C)cc21]

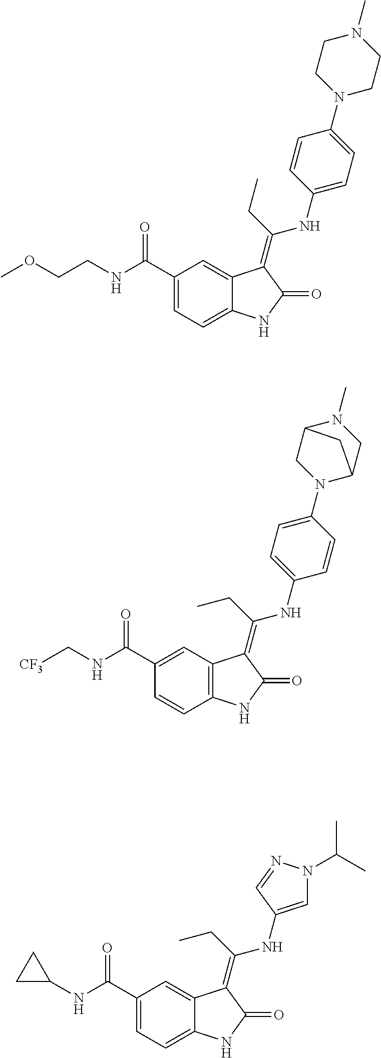 [SMILES: CC/C(Nc1ccc(N2CCN(C)CC2)cc1)=C1/C(=O)Nc2ccc(C(=O)NCCOC)cc21 ; CC/C(Nc1ccc(N2CC3CC2CN3C)cc1)=C1/C(=O)Nc2ccc(C(=O)NCC(F)(F)F)cc21 ; CC/C(Nc1cnn(C(C)C)c1)=C1/C(=O)Nc2ccc(C(=O)NC3CC3)cc21]

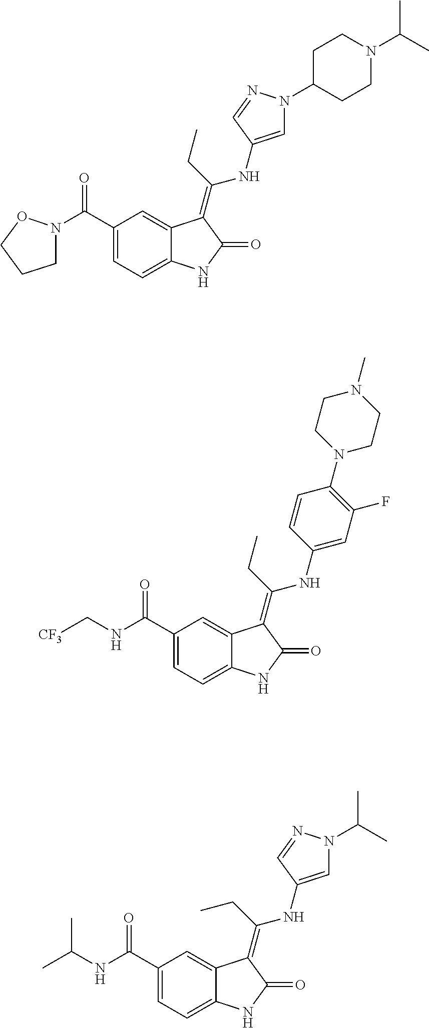

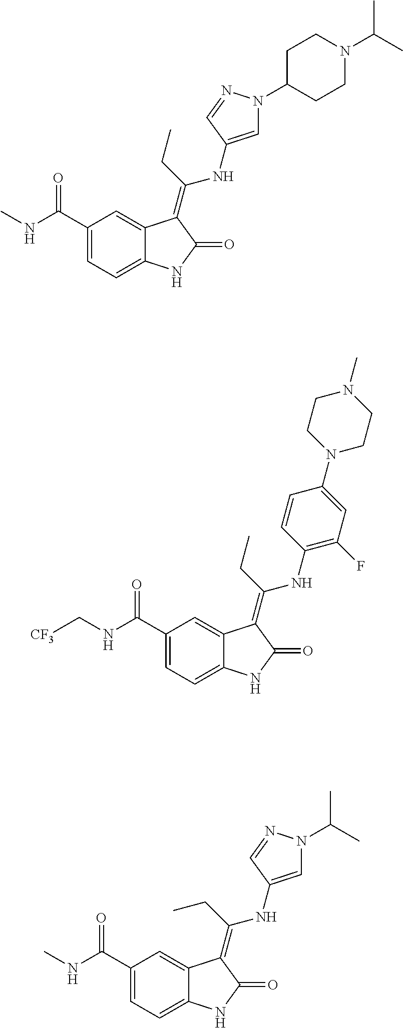

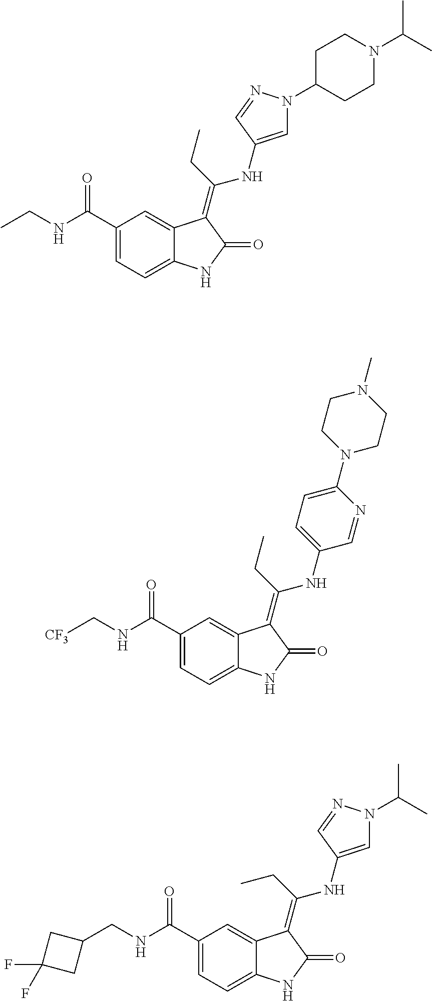 [SMILES: CCNC(=O)c1ccc2c(c1)/C(=C(\CC)Nc1cnn(C3CCN(C(C)C)CC3)c1)C(=O)N2 ; CC/C(Nc1ccc(N2CCN(C)CC2)nc1)=C1/C(=O)Nc2ccc(C(=O)NCC(F)(F)F)cc21 ; CC/C(Nc1cnn(C(C)C)c1)=C1/C(=O)Nc2ccc(C(=O)NCC3CC(F)(F)C3)cc21]

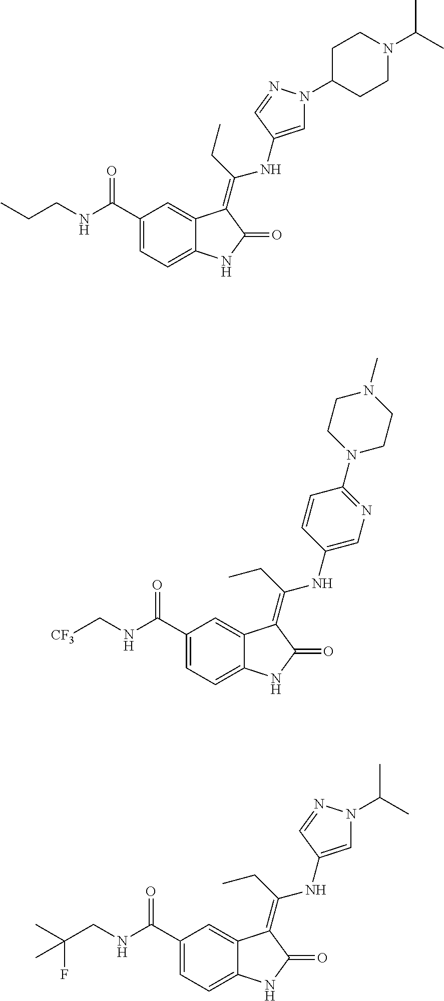

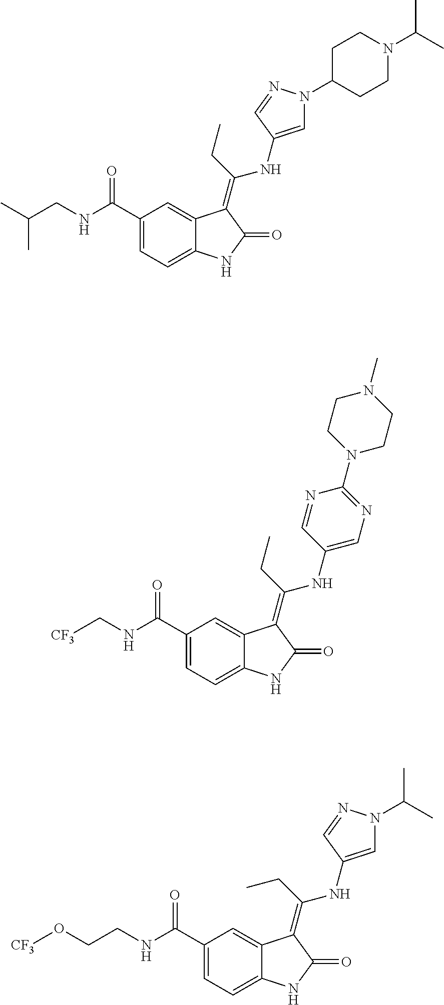 [SMILES: CC/C(Nc1cnn(C2CCN(C(C)C)CC2)c1)=C1/C(=O)Nc2ccc(C(=O)NCC(C)C)cc21 ; CC/C(Nc1cnc(N2CCN(C)CC2)nc1)=C1/C(=O)Nc2ccc(C(=O)NCC(F)(F)F)cc21 ; CC/C(Nc1cnn(C(C)C)c1)=C1/C(=O)Nc2ccc(C(=O)NCCOC(F)(F)F)cc21]

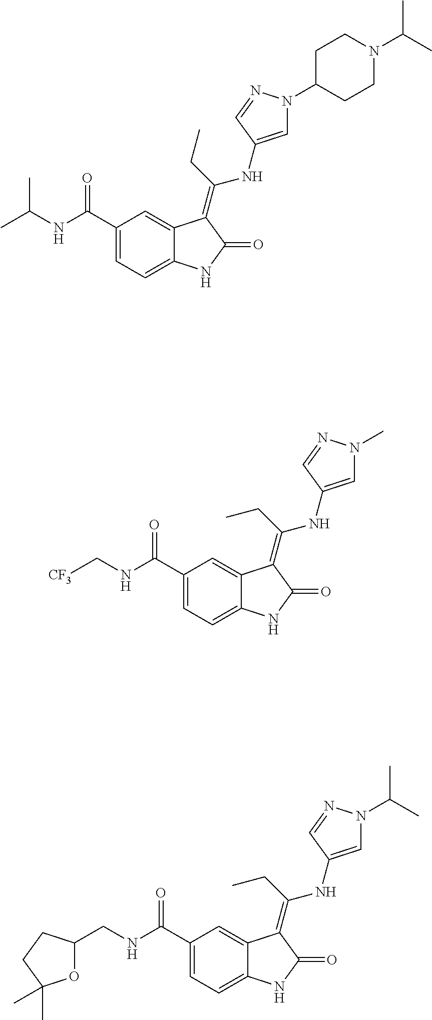

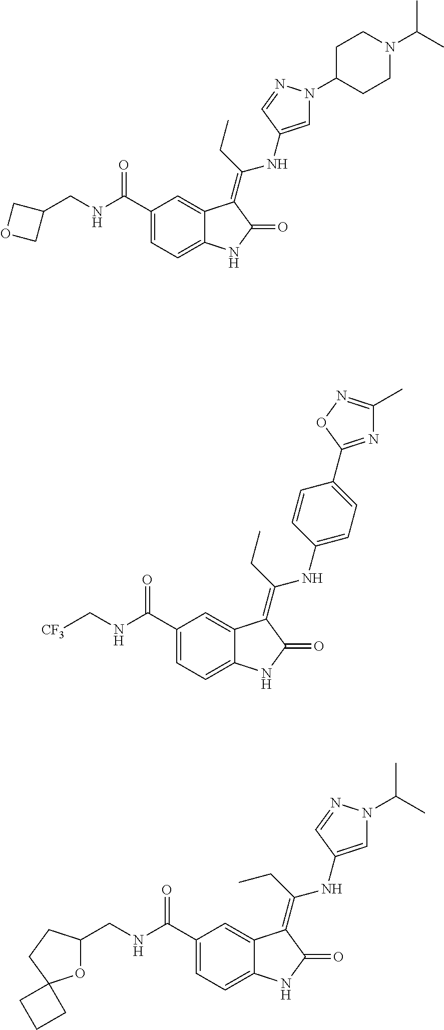 [SMILES: CC/C(Nc1cnn(C2CCN(C(C)C)CC2)c1)=C1/C(=O)Nc2ccc(C(=O)NCC3COC3)cc21 ; CC/C(Nc1ccc(-c2nc(C)no2)cc1)=C1/C(=O)Nc2ccc(C(=O)NCC(F)(F)F)cc21 ; CC/C(Nc1cnn(C(C)C)c1)=C1/C(=O)Nc2ccc(C(=O)NCC3CCC4(CCC4)O3)cc21]

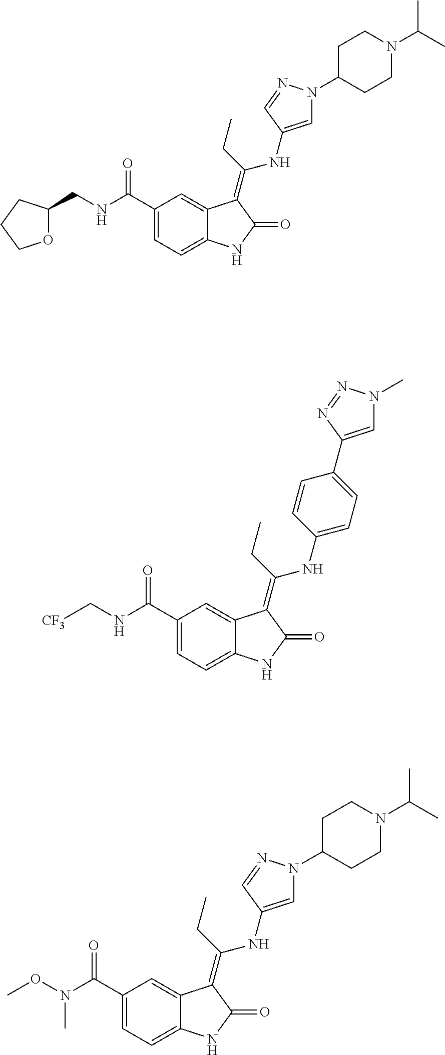

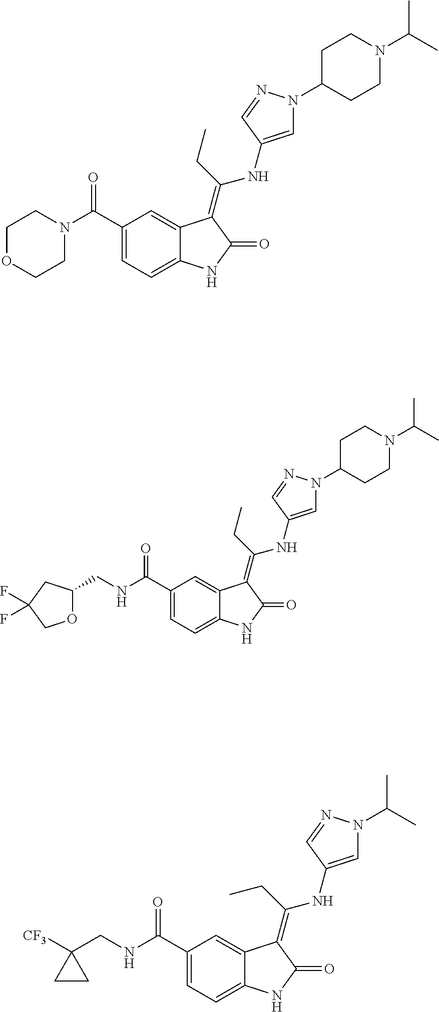 [SMILES: CC/C(Nc1cnn(C2CCN(C(C)C)CC2)c1)=C1/C(=O)Nc2ccc(C(=O)N3CCOCC3)cc21 ; CC/C(Nc1cnn(C2CCN(C(C)C)CC2)c1)=C1/C(=O)Nc2ccc(C(=O)NC[C@H]3CC(F)(F)CO3)cc21 ; CC/C(Nc1cnn(C(C)C)c1)=C1/C(=O)Nc2ccc(C(=O)NCC3(C(F)(F)F)CC3)cc21]

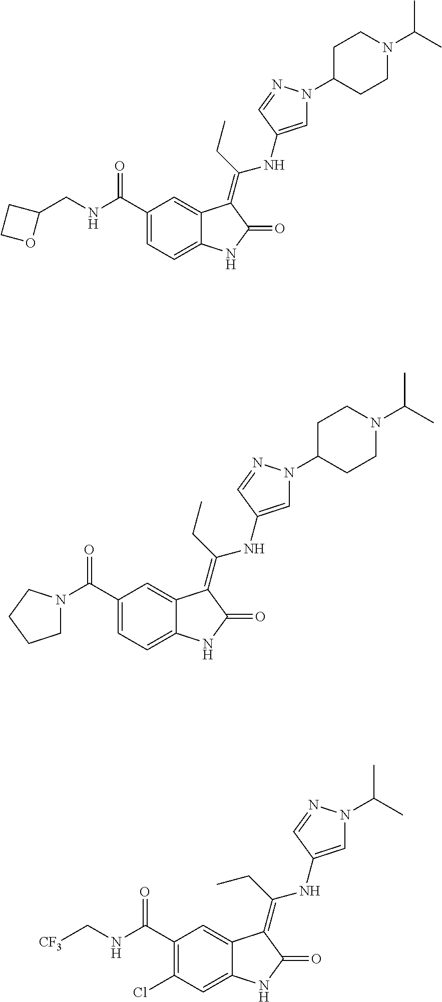 [SMILES: CC/C(Nc1cnn(C2CCN(C(C)C)CC2)c1)=C1/C(=O)Nc2ccc(C(=O)NCC3CCO3)cc21 ; CC/C(Nc1cnn(C2CCN(C(C)C)CC2)c1)=C1/C(=O)Nc2ccc(C(=O)N3CCCC3)cc21 ; CC/C(Nc1cnn(C(C)C)c1)=C1/C(=O)Nc2cc(Cl)c(C(=O)NCC(F)(F)F)cc21]

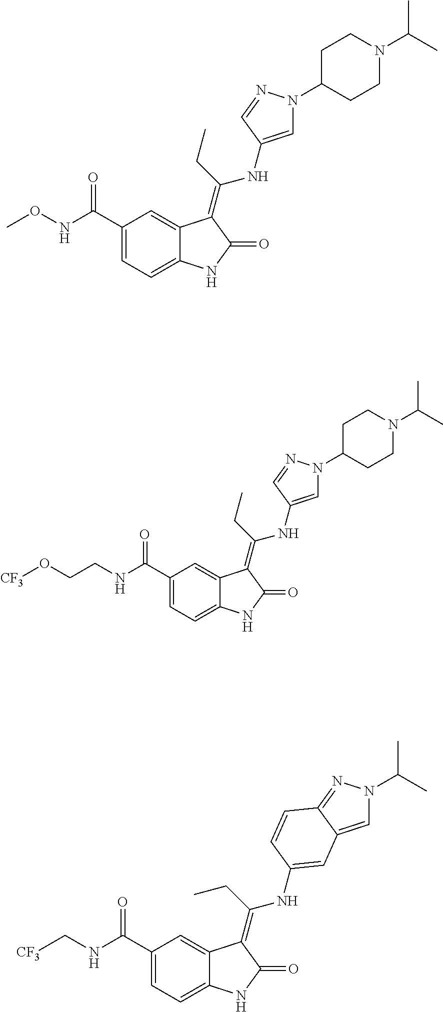 [SMILES: CC/C(Nc1cnn(C2CCN(C(C)C)CC2)c1)=C1/C(=O)Nc2ccc(C(=O)NOC)cc21 ; CC/C(Nc1cnn(C2CCN(C(C)C)CC2)c1)=C1/C(=O)Nc2ccc(C(=O)NCCOC(F)(F)F)cc21 ; CC/C(Nc1ccc2nn(C(C)C)cc2c1)=C1/C(=O)Nc2ccc(C(=O)NCC(F)(F)F)cc21]

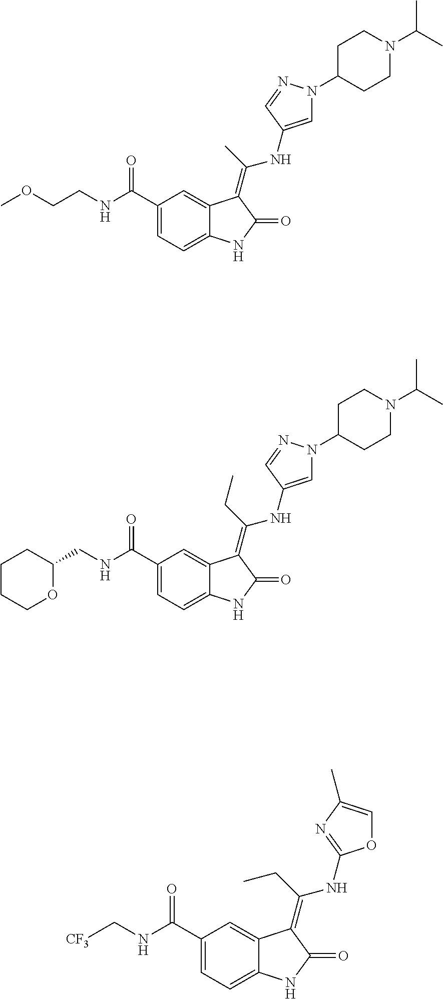 [SMILES: COCCNC(=O)c1ccc2c(c1)/C(=C(\C)Nc1cnn(C3CCN(C(C)C)CC3)c1)C(=O)N2 ; CC/C(Nc1cnn(C2CCN(C(C)C)CC2)c1)=C1/C(=O)Nc2ccc(C(=O)NC[C@H]3CCCCO3)cc21 ; CC/C(Nc1nc(C)co1)=C1/C(=O)Nc2ccc(C(=O)NCC(F)(F)F)cc21]

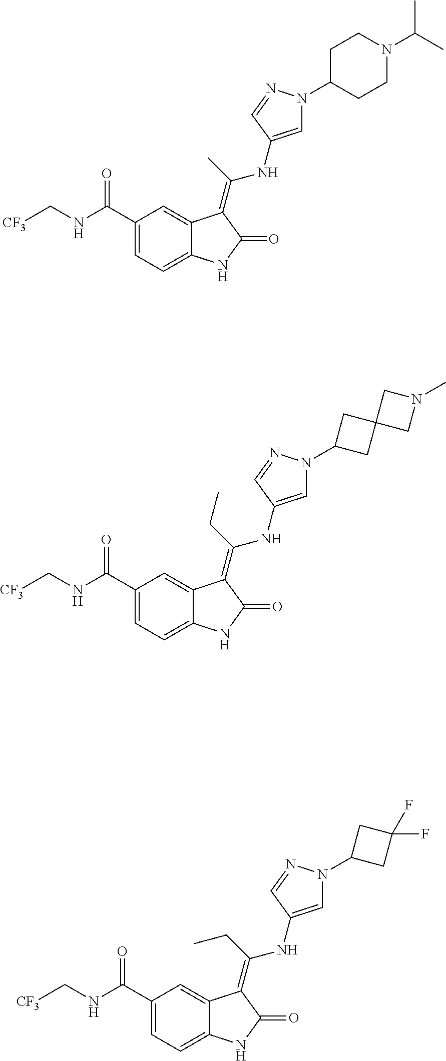 [SMILES: C/C(Nc1cnn(C2CCN(C(C)C)CC2)c1)=C1/C(=O)Nc2ccc(C(=O)NCC(F)(F)F)cc21 ; CC/C(Nc1cnn(C2CC3(C2)CN(C)C3)c1)=C1/C(=O)Nc2ccc(C(=O)NCC(F)(F)F)cc21 ; CC/C(Nc1cnn(C2CC(F)(F)C2)c1)=C1/C(=O)Nc2ccc(C(=O)NCC(F)(F)F)cc21]

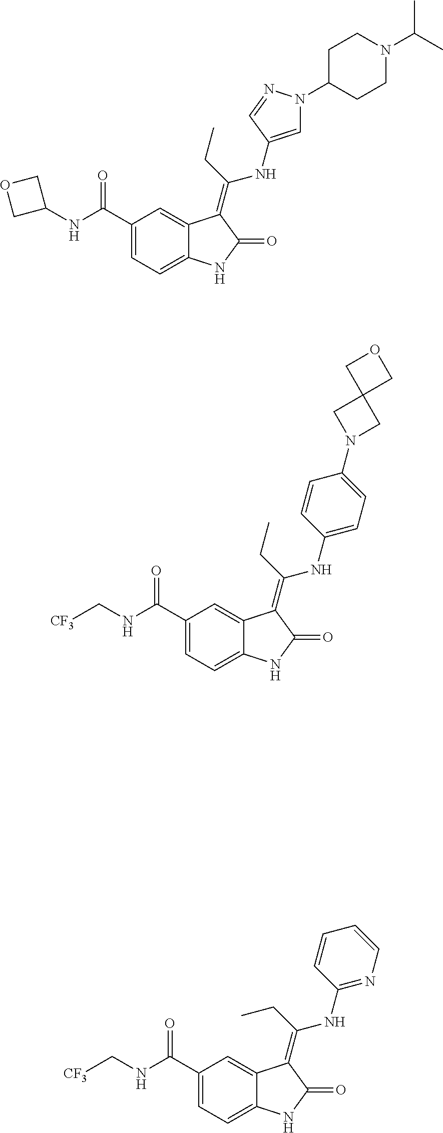 [SMILES: CC/C(Nc1cnn(C2CCN(C(C)C)CC2)c1)=C1/C(=O)Nc2ccc(C(=O)NC3COC3)cc21 ; CC/C(Nc1ccc(N2CC3(COC3)C2)cc1)=C1/C(=O)Nc2ccc(C(=O)NCC(F)(F)F)cc21 ; CC/C(Nc1ccccn1)=C1/C(=O)Nc2ccc(C(=O)NCC(F)(F)F)cc21]

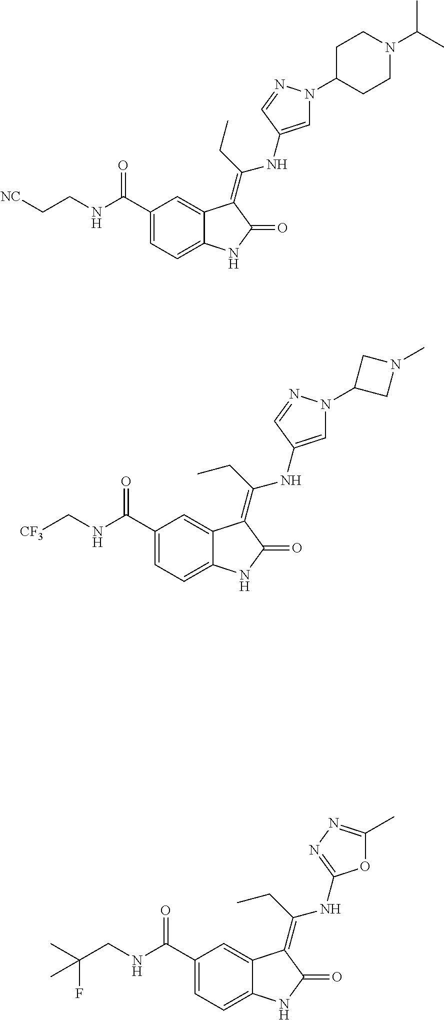

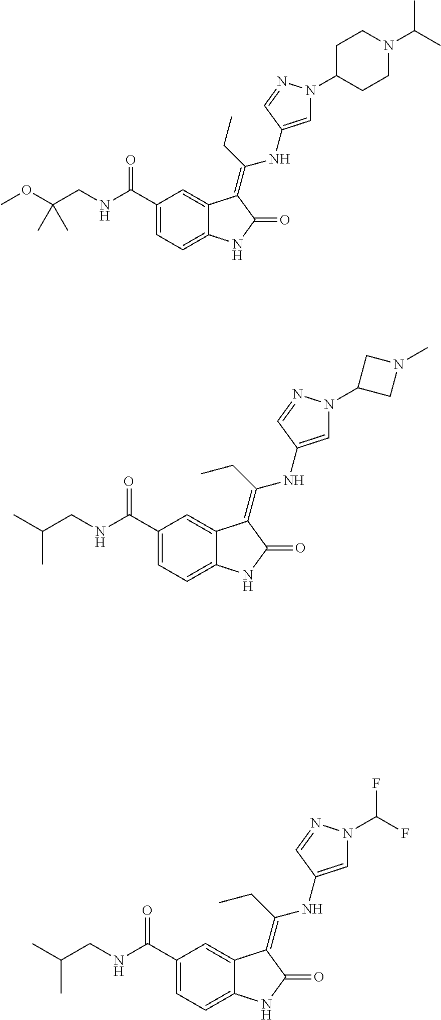

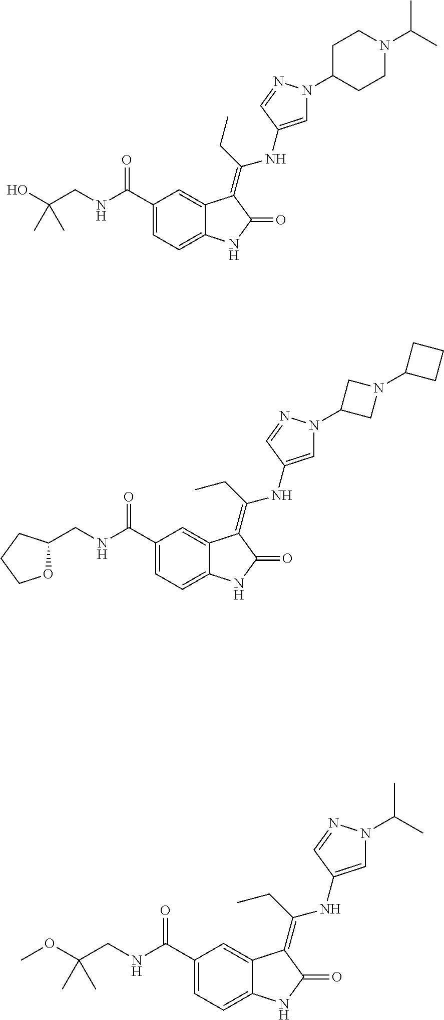

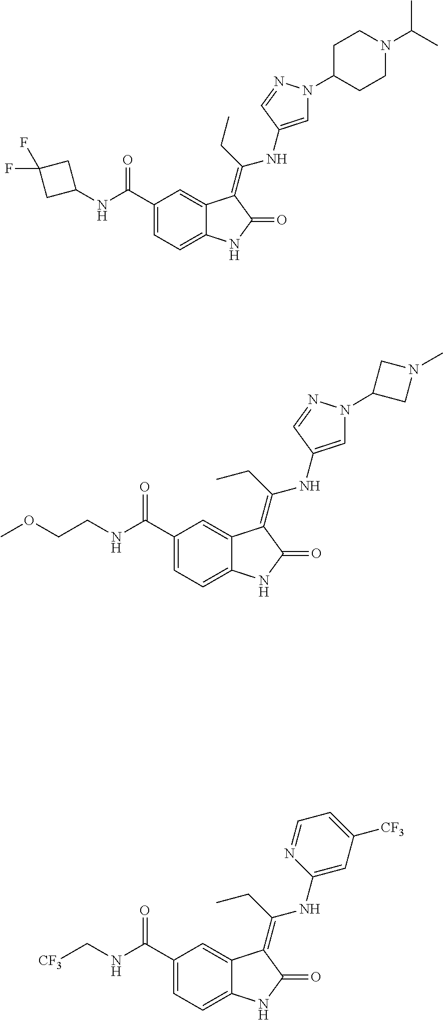

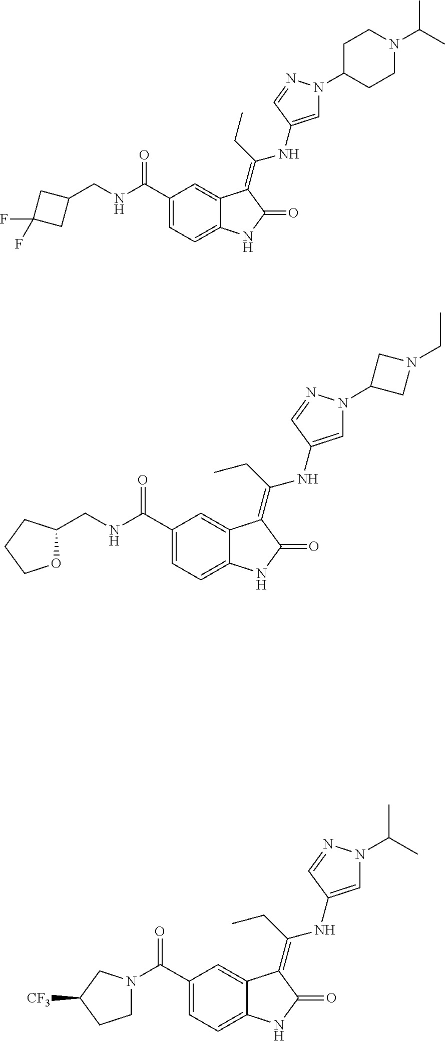 [SMILES: CC/C(Nc1cnn(C2CCN(C(C)C)CC2)c1)=C1/C(=O)Nc2ccc(C(=O)NCC3CC(F)(F)C3)cc21 ; CC/C(Nc1cnn(C2CN(CC)C2)c1)=C1/C(=O)Nc2ccc(C(=O)NC[C@H]3CCCO3)cc21 ; CC/C(Nc1cnn(C(C)C)c1)=C1/C(=O)Nc2ccc(C(=O)N3CC[C@@H](C(F)(F)F)C3)cc21]

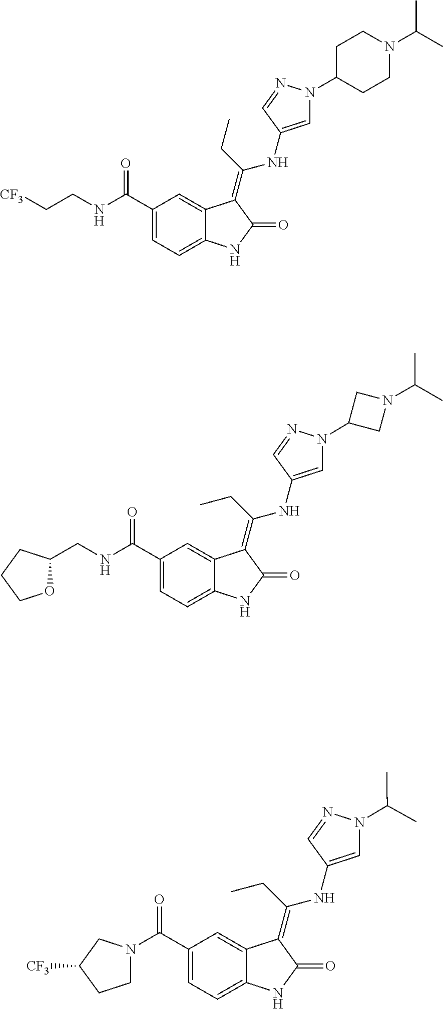 [SMILES: CC/C(Nc1cnn(C2CCN(C(C)C)CC2)c1)=C1/C(=O)Nc2ccc(C(=O)NCCC(F)(F)F)cc21 ; CC/C(Nc1cnn(C2CN(C(C)C)C2)c1)=C1/C(=O)Nc2ccc(C(=O)NC[C@H]3CCCO3)cc21 ; CC/C(Nc1cnn(C(C)C)c1)=C1/C(=O)Nc2ccc(C(=O)N3CC[C@H](C(F)(F)F)C3)cc21]

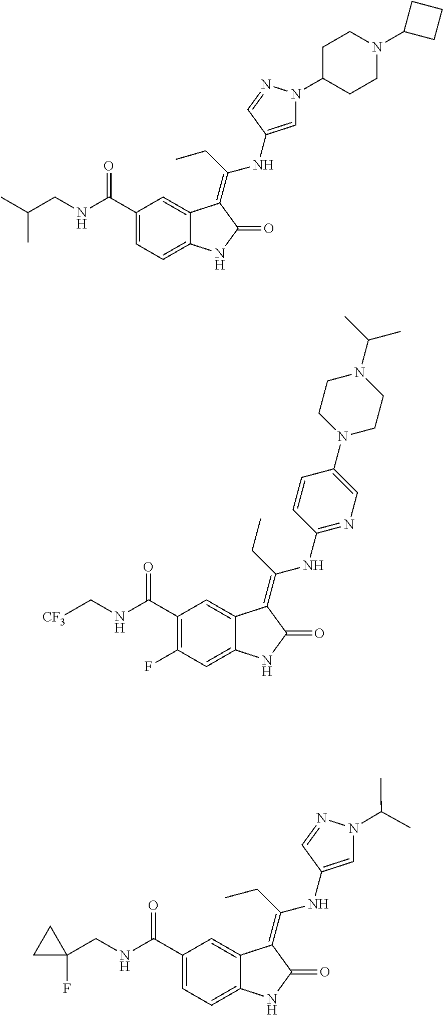 [SMILES: CC/C(Nc1cnn(C2CCN(C3CCC3)CC2)c1)=C1/C(=O)Nc2ccc(C(=O)NCC(C)C)cc21 ; CC/C(Nc1ccc(N2CCN(C(C)C)CC2)cn1)=C1/C(=O)Nc2cc(F)c(C(=O)NCC(F)(F)F)cc21 ; CC/C(Nc1cnn(C(C)C)c1)=C1/C(=O)Nc2ccc(C(=O)NCC3(F)CC3)cc21]

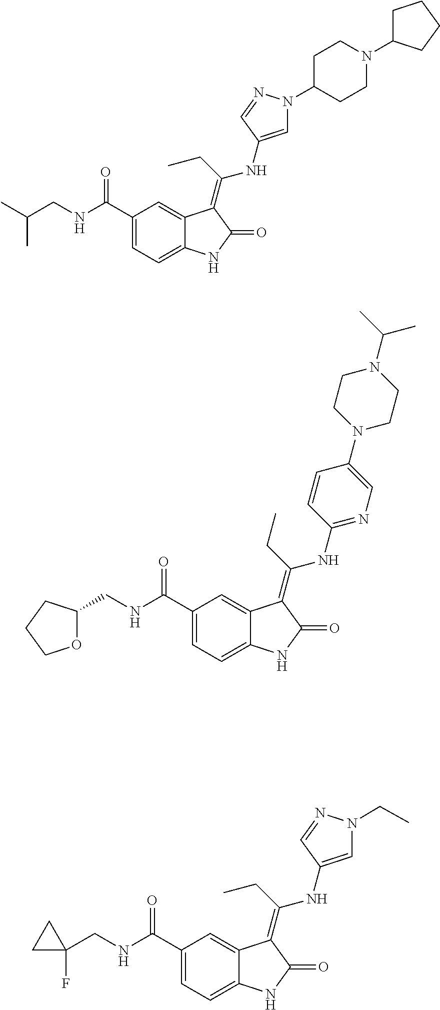 [SMILES: CC/C(Nc1cnn(C2CCN(C3CCCC3)CC2)c1)=C1/C(=O)Nc2ccc(C(=O)NCC(C)C)cc21 ; CC/C(Nc1ccc(N2CCN(C(C)C)CC2)cn1)=C1/C(=O)Nc2ccc(C(=O)NC[C@H]3CCCO3)cc21 ; CC/C(Nc1cnn(CC)c1)=C1/C(=O)Nc2ccc(C(=O)NCC3(F)CC3)cc21]

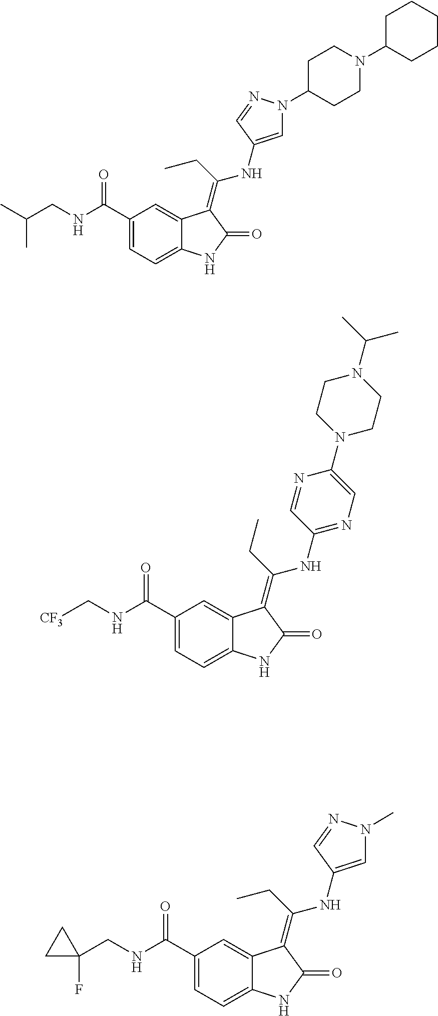 [SMILES: CC/C(Nc1cnn(C2CCN(C3CCCCC3)CC2)c1)=C1/C(=O)Nc2ccc(C(=O)NCC(C)C)cc21 ; CC/C(Nc1cnc(N2CCN(C(C)C)CC2)cn1)=C1/C(=O)Nc2ccc(C(=O)NCC(F)(F)F)cc21 ; CC/C(Nc1cnn(C)c1)=C1/C(=O)Nc2ccc(C(=O)NCC3(F)CC3)cc21]

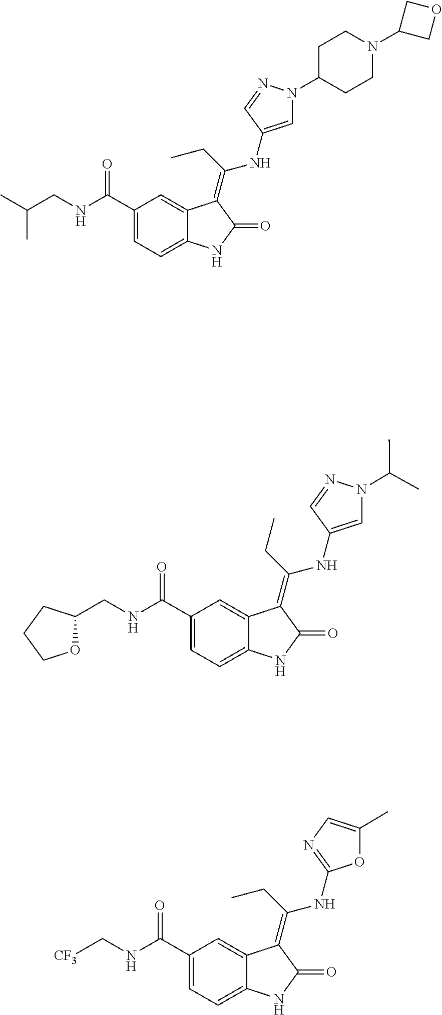 [SMILES: CC/C(Nc1cnn(C2CCN(C3COC3)CC2)c1)=C1/C(=O)Nc2ccc(C(=O)NCC(C)C)cc21 ; CC/C(Nc1cnn(C(C)C)c1)=C1/C(=O)Nc2ccc(C(=O)NC[C@H]3CCCO3)cc21 ; CC/C(Nc1ncc(C)o1)=C1/C(=O)Nc2ccc(C(=O)NCC(F)(F)F)cc21]

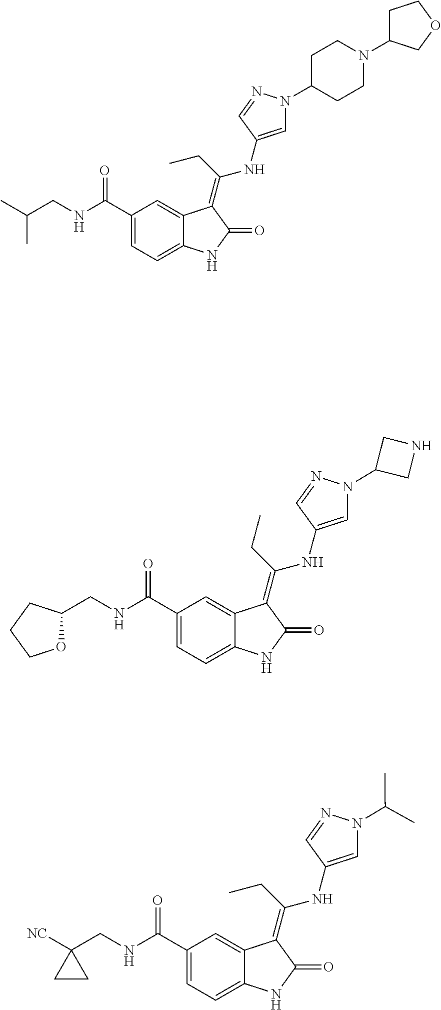 [SMILES: CC/C(Nc1cnn(C2CCN(C3CCOC3)CC2)c1)=C1/C(=O)Nc2ccc(C(=O)NCC(C)C)cc21 ; CC/C(Nc1cnn(C2CNC2)c1)=C1/C(=O)Nc2ccc(C(=O)NC[C@H]3CCCO3)cc21 ; CC/C(Nc1cnn(C(C)C)c1)=C1/C(=O)Nc2ccc(C(=O)NCC3(C#N)CC3)cc21]

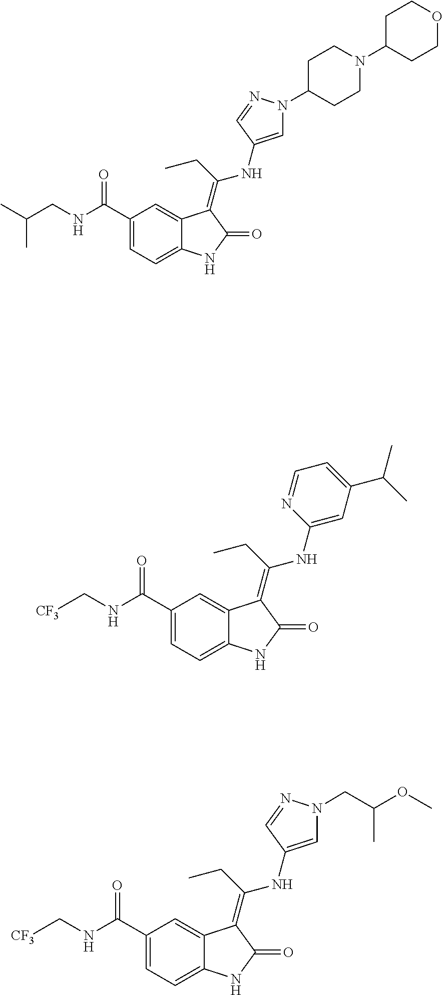 [SMILES: CC/C(Nc1cnn(C2CCN(C3CCOCC3)CC2)c1)=C1/C(=O)Nc2ccc(C(=O)NCC(C)C)cc21 ; CC/C(Nc1cc(C(C)C)ccn1)=C1/C(=O)Nc2ccc(C(=O)NCC(F)(F)F)cc21 ; CC/C(Nc1cnn(CC(C)OC)c1)=C1/C(=O)Nc2ccc(C(=O)NCC(F)(F)F)cc21]

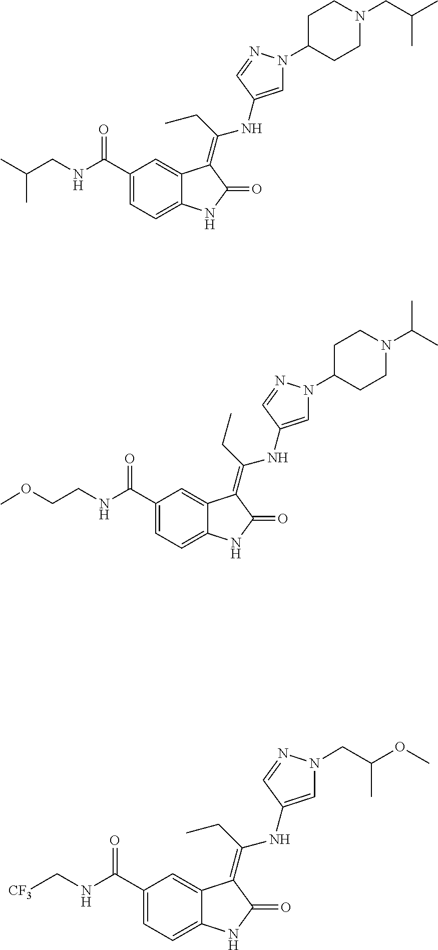

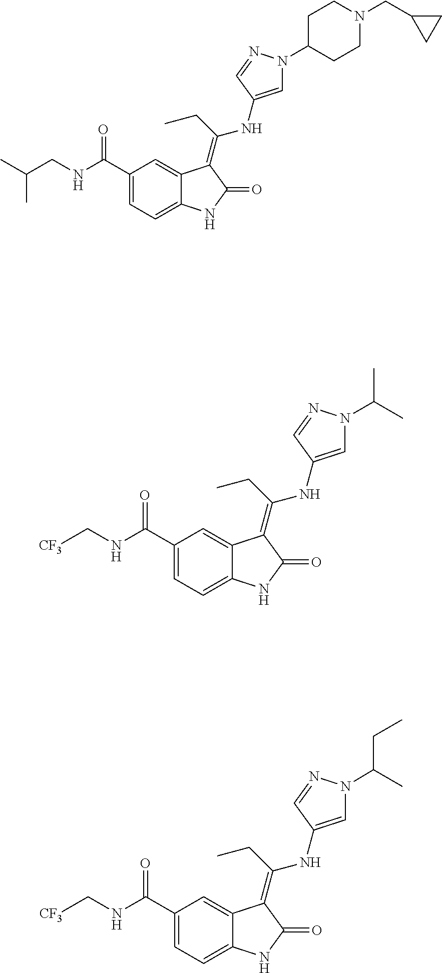 [SMILES: CC/C(Nc1cnn(C2CCN(CC3CC3)CC2)c1)=C1/C(=O)Nc2ccc(C(=O)NCC(C)C)cc21 ; CC/C(Nc1cnn(C(C)C)c1)=C1/C(=O)Nc2ccc(C(=O)NCC(F)(F)F)cc21 ; CC/C(Nc1cnn(C(C)CC)c1)=C1/C(=O)Nc2ccc(C(=O)NCC(F)(F)F)cc21]

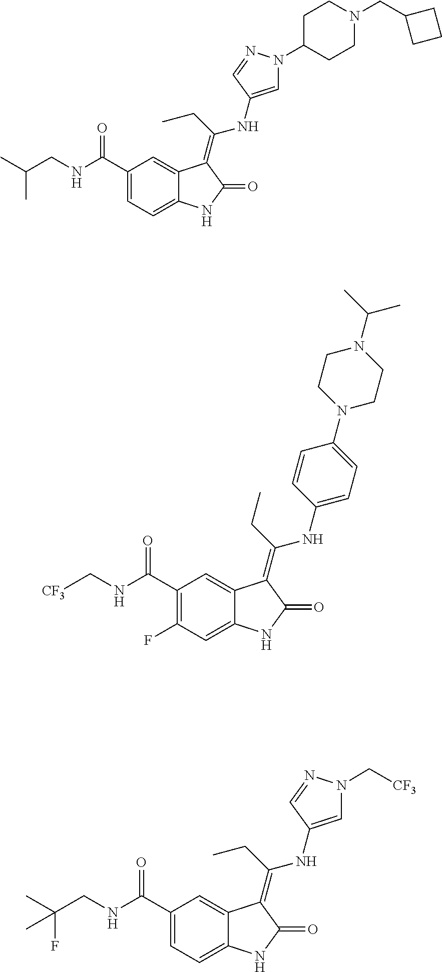

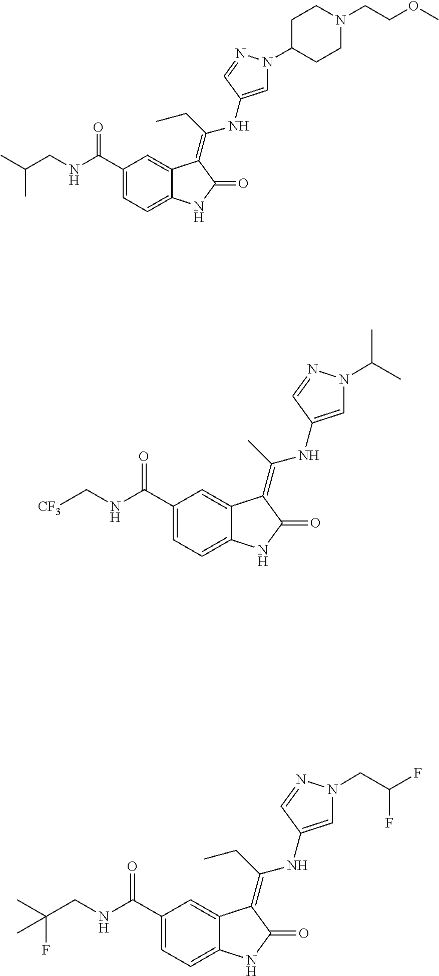

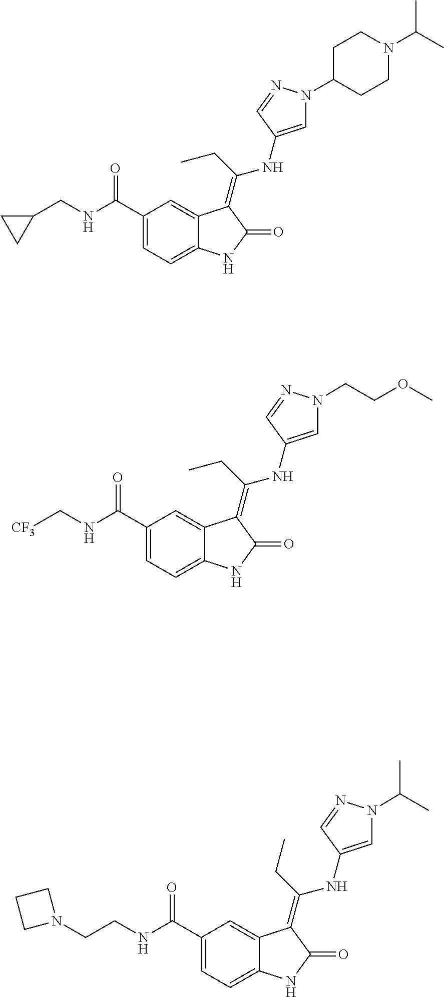 [SMILES: CC/C(Nc1cnn(C2CCN(C(C)C)CC2)c1)=C1/C(=O)Nc2ccc(C(=O)NCC3CC3)cc21 ; CC/C(Nc1cnn(CCOC)c1)=C1/C(=O)Nc2ccc(C(=O)NCC(F)(F)F)cc21 ; CC/C(Nc1cnn(C(C)C)c1)=C1/C(=O)Nc2ccc(C(=O)NCCN3CCC3)cc21]

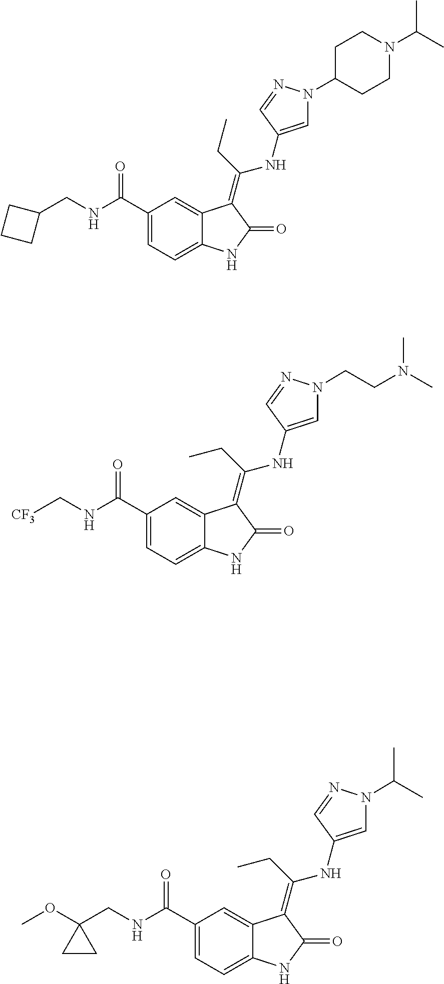

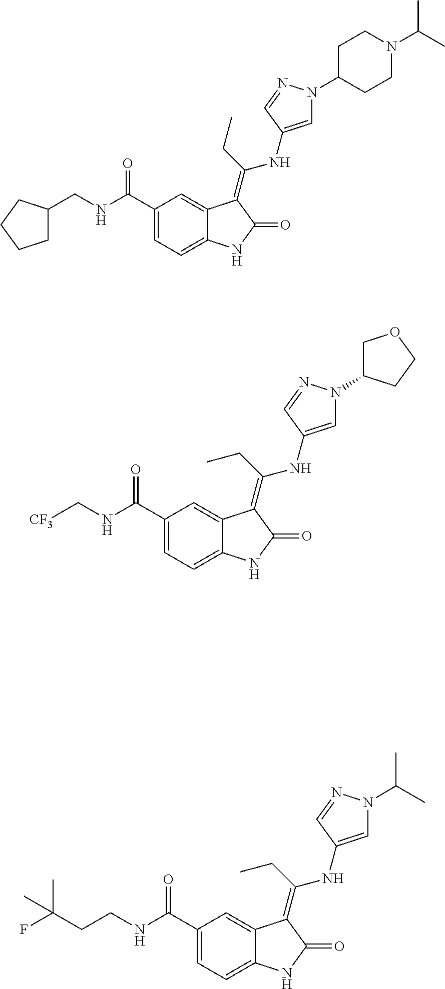 [SMILES: CC/C(Nc1cnn(C2CCN(C(C)C)CC2)c1)=C1/C(=O)Nc2ccc(C(=O)NCC3CCCC3)cc21 ; CC/C(Nc1cnn([C@H]2CCOC2)c1)=C1/C(=O)Nc2ccc(C(=O)NCC(F)(F)F)cc21 ; CC/C(Nc1cnn(C(C)C)c1)=C1/C(=O)Nc2ccc(C(=O)NCCC(C)(C)F)cc21]

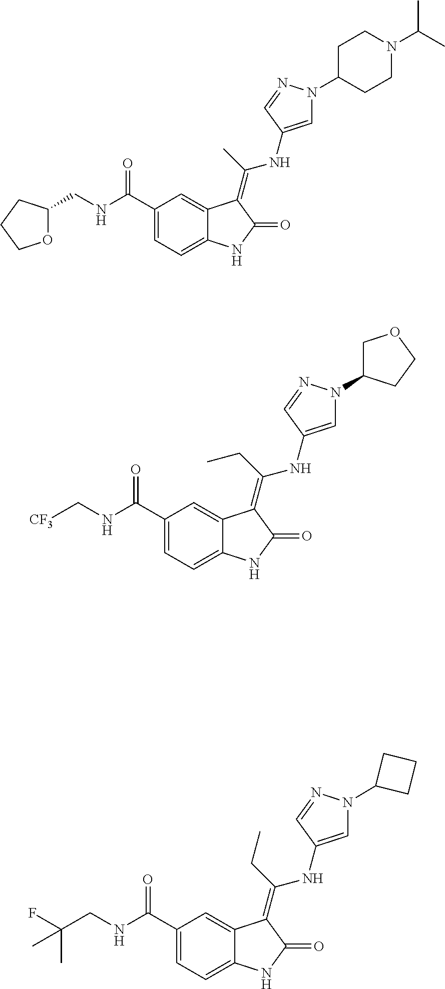 [SMILES: C/C(Nc1cnn(C2CCN(C(C)C)CC2)c1)=C1/C(=O)Nc2ccc(C(=O)NC[C@H]3CCCO3)cc21 ; CC/C(Nc1cnn([C@@H]2CCOC2)c1)=C1/C(=O)Nc2ccc(C(=O)NCC(F)(F)F)cc21 ; CC/C(Nc1cnn(C2CCC2)c1)=C1/C(=O)Nc2ccc(C(=O)NCC(C)(C)F)cc21]

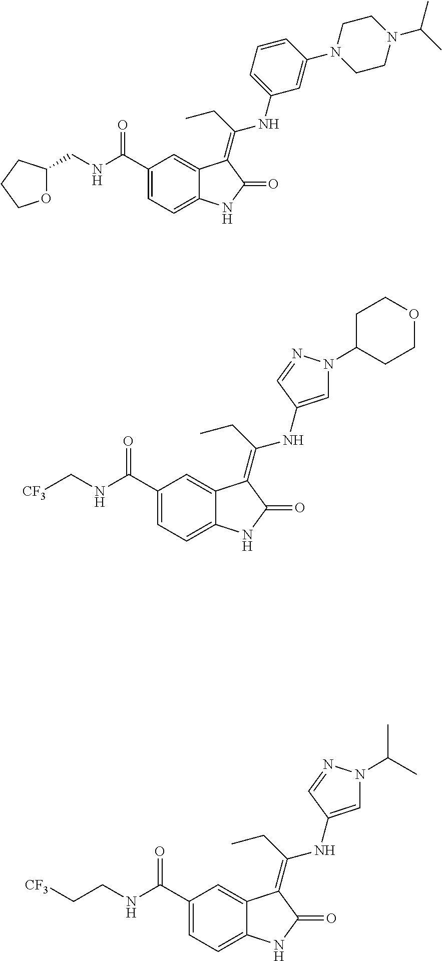 [SMILES: CC/C(Nc1cccc(N2CCN(C(C)C)CC2)c1)=C1/C(=O)Nc2ccc(C(=O)NC[C@H]3CCCO3)cc21 ; CC/C(Nc1cnn(C2CCOCC2)c1)=C1/C(=O)Nc2ccc(C(=O)NCC(F)(F)F)cc21 ; CC/C(Nc1cnn(C(C)C)c1)=C1/C(=O)Nc2ccc(C(=O)NCCC(F)(F)F)cc21]

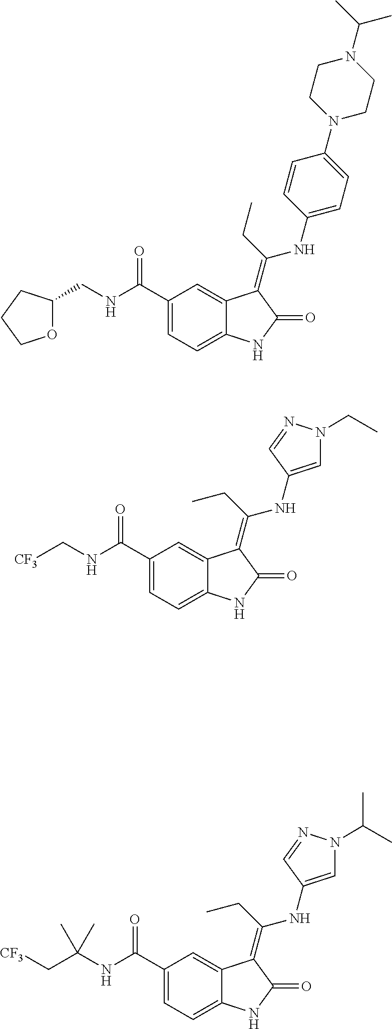 [SMILES: CC/C(Nc1ccc(N2CCN(C(C)C)CC2)cc1)=C1/C(=O)Nc2ccc(C(=O)NC[C@H]3CCCO3)cc21 ; CC/C(Nc1cnn(CC)c1)=C1/C(=O)Nc2ccc(C(=O)NCC(F)(F)F)cc21 ; CC/C(Nc1cnn(C(C)C)c1)=C1/C(=O)Nc2ccc(C(=O)NC(C)(C)CC(F)(F)F)cc21]

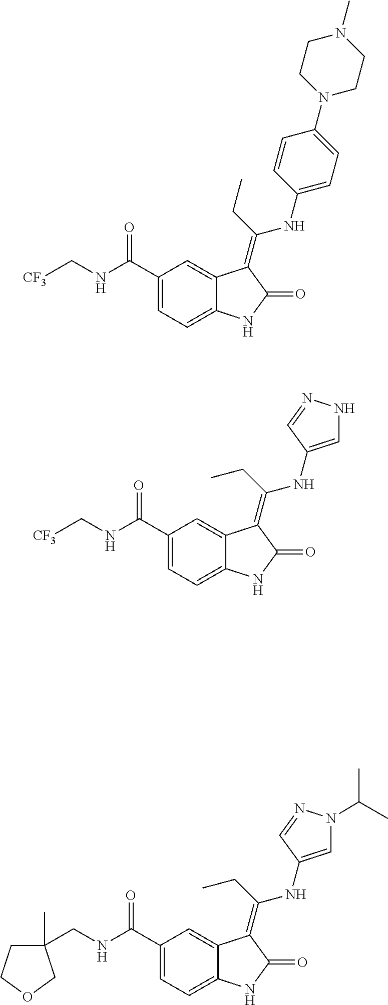 [SMILES: CC/C(Nc1ccc(N2CCN(C)CC2)cc1)=C1/C(=O)Nc2ccc(C(=O)NCC(F)(F)F)cc21 ; CC/C(Nc1cn[nH]c1)=C1/C(=O)Nc2ccc(C(=O)NCC(F)(F)F)cc21 ; CC/C(Nc1cnn(C(C)C)c1)=C1/C(=O)Nc2ccc(C(=O)NCC3(C)CCOC3)cc21]

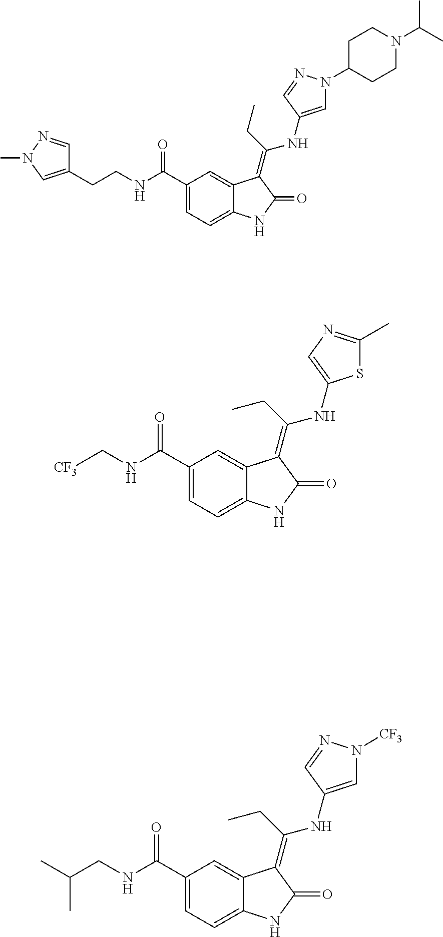

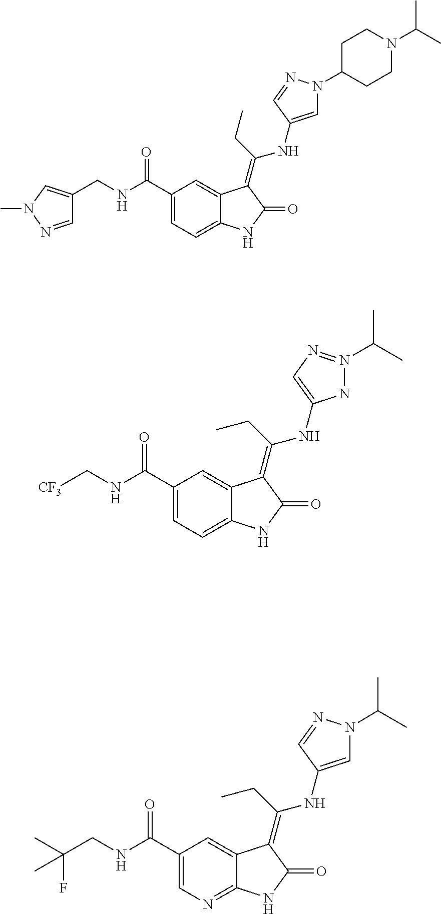

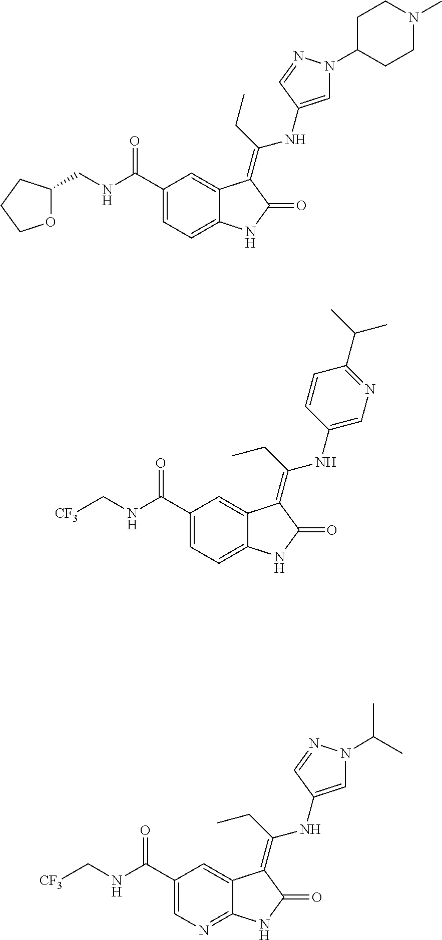 [SMILES: CC/C(Nc1cnn(C2CCN(C)CC2)c1)=C1/C(=O)Nc2ccc(C(=O)NC[C@H]3CCCO3)cc21 ; CC/C(Nc1ccc(C(C)C)nc1)=C1/C(=O)Nc2ccc(C(=O)NCC(F)(F)F)cc21 ; CC/C(Nc1cnn(C(C)C)c1)=C1/C(=O)Nc2ncc(C(=O)NCC(F)(F)F)cc21]

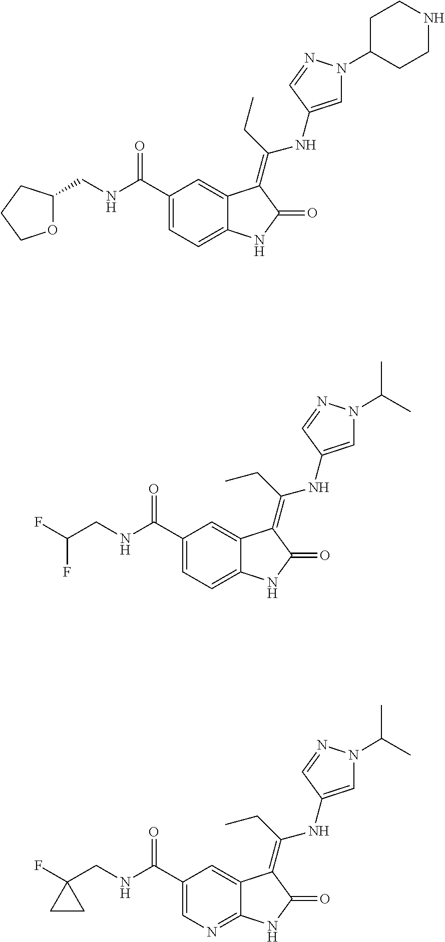 [SMILES: CC/C(Nc1cnn(C2CCNCC2)c1)=C1/C(=O)Nc2ccc(C(=O)NC[C@H]3CCCO3)cc21 ; CC/C(Nc1cnn(C(C)C)c1)=C1/C(=O)Nc2ccc(C(=O)NCC(F)F)cc21 ; CC/C(Nc1cnn(C(C)C)c1)=C1/C(=O)Nc2ncc(C(=O)NCC3(F)CC3)cc21]

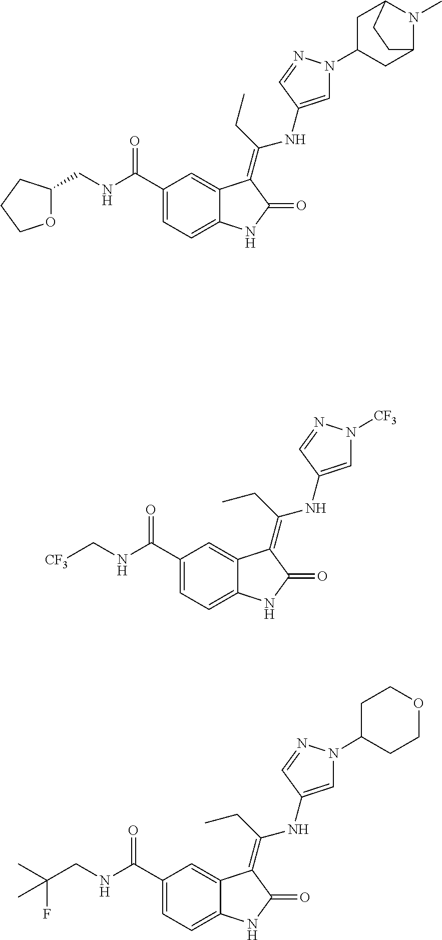

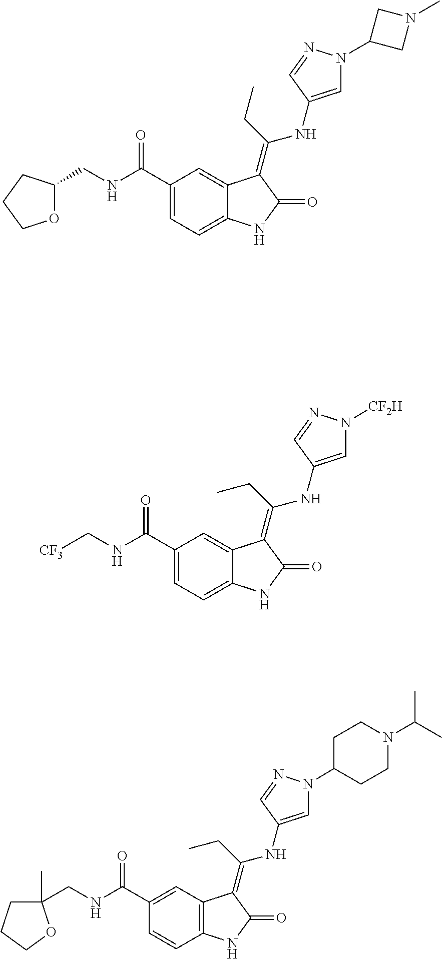 [SMILES: CC/C(Nc1cnn(C2CN(C)C2)c1)=C1/C(=O)Nc2ccc(C(=O)NC[C@H]3CCCO3)cc21 ; CC/C(Nc1cnn(C(F)F)c1)=C1/C(=O)Nc2ccc(C(=O)NCC(F)(F)F)cc21 ; CC/C(Nc1cnn(C2CCN(C(C)C)CC2)c1)=C1/C(=O)Nc2ccc(C(=O)NCC3(C)CCCO3)cc21]

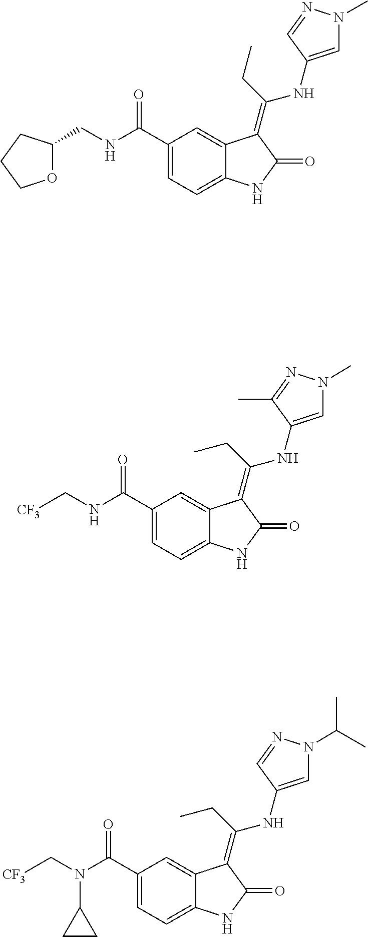 [SMILES: CC/C(Nc1cnn(C)c1)=C1/C(=O)Nc2ccc(C(=O)NC[C@H]3CCCO3)cc21 ; CC/C(Nc1cn(C)nc1C)=C1/C(=O)Nc2ccc(C(=O)NCC(F)(F)F)cc21 ; CC/C(Nc1cnn(C(C)C)c1)=C1/C(=O)Nc2ccc(C(=O)N(CC(F)(F)F)C3CC3)cc21]

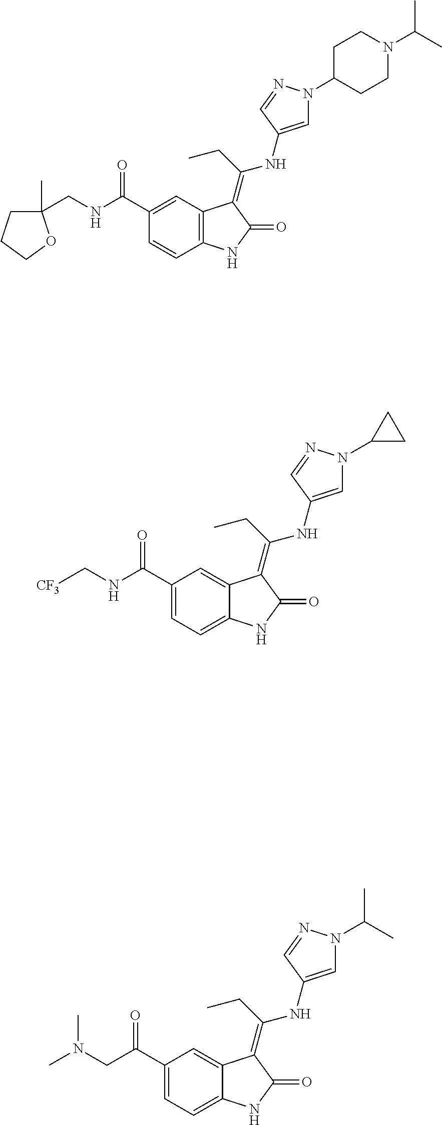

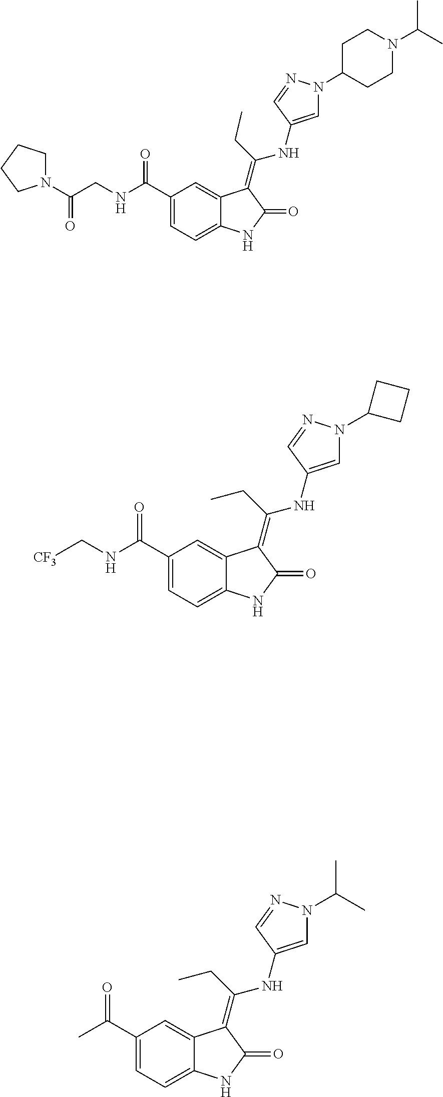

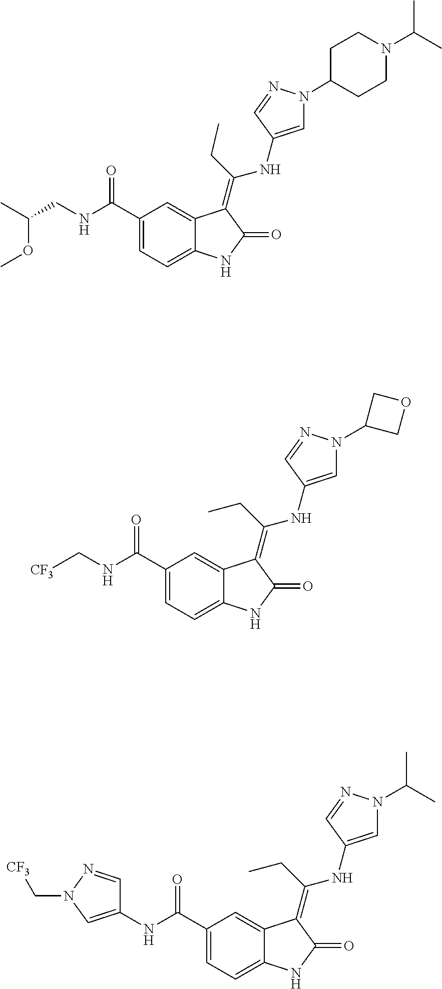 [SMILES: CC/C(Nc1cnn(C2CCN(C(C)C)CC2)c1)=C1/C(=O)Nc2ccc(C(=O)NC[C@@H](C)OC)cc21 ; CC/C(Nc1cnn(C2COC2)c1)=C1/C(=O)Nc2ccc(C(=O)NCC(F)(F)F)cc21 ; CC/C(Nc1cnn(C(C)C)c1)=C1/C(=O)Nc2ccc(C(=O)Nc3cnn(CC(F)(F)F)c3)cc21]

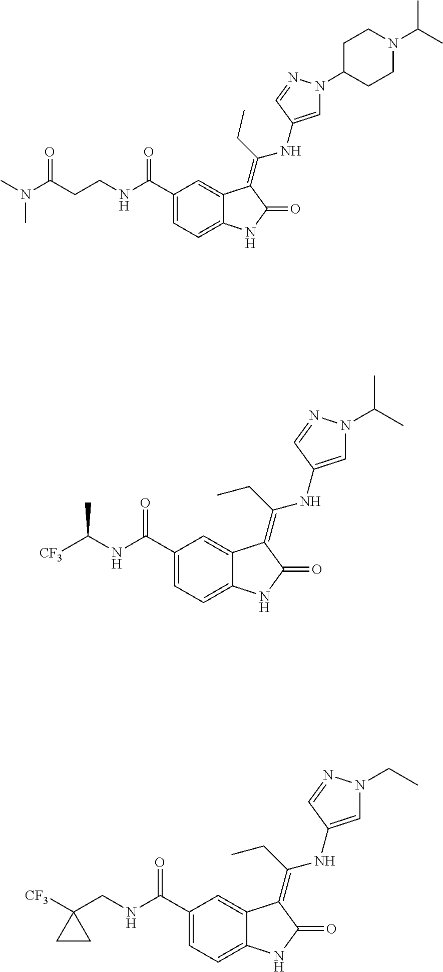 [SMILES: CC/C(Nc1cnn(C2CCN(C(C)C)CC2)c1)=C1/C(=O)Nc2ccc(C(=O)NCCC(=O)N(C)C)cc21 ; CC/C(Nc1cnn(C(C)C)c1)=C1/C(=O)Nc2ccc(C(=O)N[C@H](C)C(F)(F)F)cc21 ; CC/C(Nc1cnn(CC)c1)=C1/C(=O)Nc2ccc(C(=O)NCC3(C(F)(F)F)CC3)cc21]

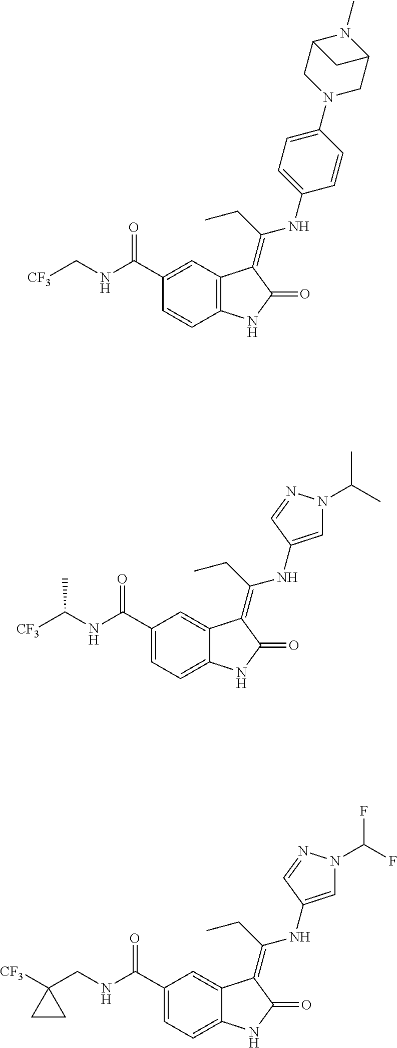 [SMILES: CC/C(Nc1ccc(N2CC3CC(C2)N3C)cc1)=C1/C(=O)Nc2ccc(C(=O)NCC(F)(F)F)cc21 ; CC/C(Nc1cnn(C(C)C)c1)=C1/C(=O)Nc2ccc(C(=O)N[C@@H](C)C(F)(F)F)cc21 ; CC/C(Nc1cnn(C(F)F)c1)=C1/C(=O)Nc2ccc(C(=O)NCC3(C(F)(F)F)CC3)cc21]

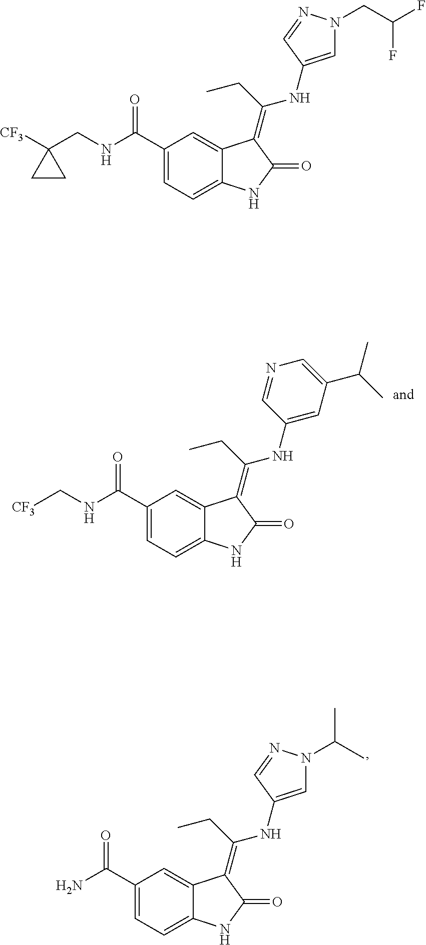 [SMILES: CC/C(Nc1cnn(CC(F)F)c1)=C1/C(=O)Nc2ccc(C(=O)NCC3(C(F)(F)F)CC3)cc21 ; CC/C(Nc1cncc(C(C)C)c1)=C1/C(=O)Nc2ccc(C(=O)NCC(F)(F)F)cc21 ; CC/C(Nc1cnn(C(C)C)c1)=C1/C(=O)Nc2ccc(C(N)=O)cc21]

and pharmaceutically acceptable salts, enantiomers, stereoisomers, or tautomers thereof.

### Methods of Treatment

Compounds described herein (e.g., compounds of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, and Formula XVII, or a pharmaceutically acceptable salt, enantiomer, stereoisomer, or tautomer thereof) can act as inhibitors of autophagy useful in the treatment of a disorder in a patient in need thereof. The disorder, for example, can be a tumor, e.g., a solid tumor. The disorder may also be cancer.

Exemplary disorders also include gastrointestinal stromal tumors, esophageal cancer, gastric cancer, melanomas, gliomas, glioblastomas, ovarian cancer, bladder cancer, pancreatic cancer, prostate cancer, lung cancers, breast cancers, renal cancers, hepatic cancers, osteosarcomas, multiple myelomas, leukemias, cervical carcinomas, cancers that are metastatic to bone, papillary thyroid carcinoma, non-small cell lung cancer, and colorectal cancers. A cancer treated by the methods described herein may be a metastatic cancer.

In some embodiments, the compounds described herein are useful for the treatment of cancers caused by RAS mutation. In some embodiments, the cancer is caused by a KRAS mutation. In some embodiments, the cancer has additional mutations in tumor suppressor proteins, including mutations in TP53, PTEN, CDN2A/INK4A, p16, or STAG2. In some embodiments, these additional mutations occur in one or more of TP53, PTEN, CDN2A/INK4A, p16, or STAG2. In some embodiments, the cancer is pancreatic ductal adenocarcinoma. In some embodiments, the cancer is lung cancer. In some embodiments, the cancer is colorectal.

In some embodiments, determination of cellular inhibition of autophagy by compounds described herein is determined by monitoring of autophagic flux, for instance by monitoring inhibition of autophagy-mediated clearance of mCherry/GFP-LC3 fusion protein. In some embodiments, determination of cellular inhibition of autophagy by compounds described herein is determined by monitoring of accumulation of autophagic proteins such as p62 or LC-3. In some embodiments, determination of cellular inhibition of autophagy by compounds described herein is determined by decreased clearance of luciferase-tagged LC3 protein. In some embodiments, determination of cellular inhibition of autophagy by compounds described herein is determined by monitoring decreases in cellular autophagosomes, for instance by measurement of fluorescent puncta with the autophagosome marker Cyto-ID.

In some embodiments, cellular inhibition of ULK kinase by compounds described herein is determined by inhibition of phosphorylation of cellular ULK substrates including ATG13, ATG14, Beclin 1, or STING either in tumor cells or in non-tumor host tissues. In some embodiments, cellular inhibition of ULK kinase by compounds described herein is determined in host tissues including immune cells.

In some embodiments, in vivo inhibition of autophagy by compounds described herein is determined by inhibition of phosphorylation of cellular ULK substrates including ATG13, ATG14, Beclin 1, or STING either in tumor cells or in non-tumor host tissues. In some embodiments, in vivo inhibition of ULK kinase by compounds described herein is determined in host tissues including immune cells. In some embodiments, the in vivo inhibition of autophagic flux by compounds described herein can be used as a pharmacodynamic model for monitoring the kinetics and extent of such ULK inhibition. In some embodiments, tin vivo inhibition of ULK kinase by compounds described herein is determined in pancreatic cancer-bearing animals. In some embodiments, in vivo inhibition of ULK kinase by compounds described herein is determined in lung cancer-bearing animals. In some embodiments, in vivo inhibition of ULK kinase is determined in colorectal cancer-bearing animals. In some embodiments, in vivo inhibition of autophagy by compounds described herein is determined by inhibition of autophagic flux in tumor cells, or in non-tumor host tissues by monitoring inhibition of autophagosome formation, or by accumulation of autophagic proteins such as p62 or LC-III. In some embodiments, in vivo inhibition of autophagy is determined in host tissues including immune cells. In some embodiments, the in vivo inhibition of autophagic flux can be used as a pharmacodynamic model for monitoring the kinetics and extent of such ULK inhibition.

In some embodiments, inhibition of autophagy and anti-tumor activity by compounds described herein are evaluated in xenograft studies utilizing human RAS mutant cell lines in immunocompromised mice, for instance in SCID or nude mice. In some embodiments, inhibition of autophagy and anti-tumor activity by compounds described herein are evaluated in xenograft studies utilizing human RAS mutant patient-derived tumor xenografts (PDXs) in immunocompromised mice, for instance in SCID or nude mice. In some embodiments, xenograft studies include evaluation of compounds described herein in pancreatic cancer models. In some embodiments, inhibition of autophagy and anti-tumor activity by compounds described herein are evaluated in syngeneic murine genetically engineered models (GEMs) of mutant RAS cancers. In some embodiments, inhibition of autophagy and anti-tumor activity by compounds described herein are evaluated in the murine GEM syngeneic orthotopic pancreatic cancer model known as the KPC model (LSL-Kras^{G12D/+}; LSL-Trp53^{R172H/+}; Pdx-1-Cre) or variants of the KPC model.

In some embodiments, compounds described herein will be evaluated in xenograft or GEM cancer models in combination with a MEK inhibitor. In some embodiments, compounds described herein will be evaluated in xenograft or GEM cancer models in combination with a RAF inhibitor. In some embodiments, compounds described herein will be evaluated in xenograft or GEM cancer models in combination with an ERK inhibitor. In some embodiments, compounds described herein will be evaluated in xenograft or GEM cancer models in combination with a RAS G12C direct inhibitor.

In some embodiments, inhibition of autophagy and anti-tumor activity by compounds described herein is evaluated in immunocompetent murine cancer models to assess an immunomodulatory component to the mechanism of action of ULK inhibitors. In some embodiments, the immunocompetent murine model is the murine GEM syngeneic orthotopic pancreatic cancer model known as the KPC model (LSL-Kras^{G12D/+}; LSL-Trp53^{R172H/+}; Pdx-1-Cre) or variants of the KPC model. In some embodiments, immunomodulatory properties of compounds described herein are evaluated in combination with a MEK inhibitor. In some embodiments, immunomodulatory properties of compounds described herein are evaluated in combination with a RAF inhibitor. In some embodiments, immunomodulatory properties of compounds described herein are evaluated in combination with an ERK inhibitor. In some embodiments, immunomodulatory properties of compounds described herein are evaluated in combination with a RAS G12C direct inhibitor.

In some embodiments, the immunomodulatory component of ULK inhibition is an enhanced innate immune response. In some embodiments, the immunomodulatory component of ULK inhibition is an enhanced adaptive immune response. In some embodiments, the immunomodulatory component of ULK inhibition is an enhanced activity of antigen-presenting cells. In some embodiments, the immunomodulatory component of ULK inhibition is an enhanced anti-tumor activity of myeloid cells including macrophages. In some embodiments, the immunomodulatory component of ULK inhibition is an enhanced anti-tumor activity of Natural Killer cells. In some embodiments, the immunomodulatory component of ULK inhibition is an enhanced activity of effector T Cells, including cytotoxic T Cells.

In an embodiment, provided herein is a method of treating a disorder described herein that includes: administering a therapeutically effective amount of compound described herein in a patient in need thereof, and during or after the course of administration (e.g., at discrete time points, such as one week, two weeks, or on month after initial administration of a contemplated compound) detecting the engagement of the compound with an ULK kinase, wherein detecting comprises contacting a sample obtained from the patient (including but not limited to a tumor, blood, saliva, or tissue) with a phospho-ATG13 antibody ELISA assay to detect inhibition of ULK kinase activity, e.g., based on the level of phospho-ATG13 in the sample. In some embodiments, a contemplated method comprises optionally contacting a sample obtained from the patient (including but not limited to a tumor, blood, saliva, or tissue) prior to administration of the compound with a phospho-ATG13 antibody ELISA assay, and comparing the level of phospho-ATG13 in the sample obtained prior to administration with the level of phospho-ATG13 in the sample obtained during or after the course of administration. In some embodiments, the phospho-ATG13 is p-S318ATG13.

In an embodiment, provided herein is a method of treating a disorder described herein that includes: administering a therapeutically effective amount of compound described herein in a patient in need thereof, and during or after the course of administration (e.g., at discrete time points, such as one week, two weeks, or on month after initial administration of a contemplated compound) detecting the engagement of the compound with an ULK kinase, wherein detecting comprises contacting a sample obtained from the patient (including but not limited to a tumor, blood, saliva, or tissue) with a phospho-ATG14 antibody ELISA assay to detect inhibition of ULK kinase activity, e.g., based on the level of phospho-ATG14 in the sample. In some embodiments, a contemplated method comprises optionally contacting a sample obtained from the patient (including but not limited to a tumor, blood, saliva, or tissue) prior to administration of the compound with a phospho-ATG14 antibody ELISA assay, and comparing the level of phospho-ATG14 in the sample obtained prior to administration with the level of phospho-ATG14 in the sample obtained during or after the course of administration. In some embodiments, the phospho-ATG14 is p-ATG14 Ser29.

In an embodiment, provided herein is a method of treating a disorder described herein that includes: administering a therapeutically effective amount of compound described herein in a patient in need thereof, and during or after the course of administration (e.g., at discrete time points, such as one week, two weeks, or on month after initial administration of a contemplated compound) detecting the engagement of the compound with an ULK kinase, wherein detecting comprises contacting a sample obtained from the patient (including but not limited to a tumor, blood, saliva, or tissue) with a p62 antibody ELISA assay to detect inhibition of ULK kinase activity, e.g., based on the level of p62 in the sample. In some embodiments, a contemplated method comprises optionally contacting a sample obtained from the patient (including but not limited to a tumor, blood, saliva, or tissue) prior to administration of the compound with a p62 antibody ELISA assay, and comparing the level of p62 in the sample obtained prior to administration with the level of p62 in the sample obtained during or after the course of administration.

In an embodiment, provided herein is a method of treating a disorder described herein that includes: administering a therapeutically effective amount of compound described herein in a patient in need thereof, and during or after the course of administration (e.g., at discrete time points, such as one week, two weeks, or on month after initial administration of a contemplated compound) detecting the engagement of the compound with an ULK kinase, wherein detecting comprises contacting a sample obtained from the patient (including but not limited to a tumor, blood, saliva, or tissue) with a pBeclin antibody ELISA assay to detect inhibition of ULK kinase activity, e.g, based on the level of pBeclin in the sample. In some embodiments, a contemplated method comprises optionally contacting a sample obtained from the patient (including but not limited to a tumor, blood, saliva, or tissue) prior to administration of the compound with a pBeclin antibody ELISA assay, and comparing the level of pBeclin in the sample obtained prior to administration with the level of pBeclin in the sample obtained during or after the course of administration.

The compounds provided herein may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound provided herein may be administered orally, subcutaneously, topically, parenterally, by inhalation spray or rectally in dosage unit formulations containing conventional non-toxic pharmaceutically acceptable carriers, adjuvants and vehicles. Parenteral administration may include subcutaneous injections, intravenous or intramuscular injections or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result is achieved.

### Combination Therapy

Compounds described herein, e.g., a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, enantiomer, stereoisomer, or tautomer thereof, as defined herein, can be administered in combination with one or more additional therapeutic agents to treat a disorder described herein, such as a cancer described herein. For example, provided in the present disclosure is a pharmaceutical composition comprising a compound described herein, e.g., a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, enantiomer, stereoisomer, or tautomer thereof, as defined herein, one or more additional therapeutic agents, and a pharmaceutically acceptable excipient. In some embodiments, a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, enantiomer, stereoisomer, or tautomer thereof, as defined herein, and one additional therapeutic agent is administered. In some embodiments, a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, enantiomer, stereoisomer, or tautomer thereof, as defined herein, and two additional therapeutic agents are administered. In some embodiments, a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, enantiomer, stereoisomer, or tautomer thereof, as defined herein, and three additional therapeutic agents are administered. Combination therapy can be achieved by administering two or more therapeutic agents, each of which is formulated and administered separately. For example, a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, enantiomer, stereoisomer, or tautomer thereof, as defined herein, and an additional therapeutic agent can be formulated and administered separately. Combination therapy can also be achieved by administering two or more therapeutic agents in a single formulation, for example a pharmaceutical composition comprising a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, enantiomer, stereoisomer, or tautomer thereof, as one therapeutic agent and one or more additional therapeutic agents such as a chemotherapeutic agent. For example, a compound of Formula I, Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, enantiomer, stereoisomer, or tautomer thereof, as defined herein, and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases, even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X—Y—X, X-X—Y, Y—X—Y, Y—Y—X, X-X—Y—Y, etc.

Combination therapy can also include two or more administrations of one or more of the agents used in the combination using different routes of administration. Each of the one or more of the agents may be independently administered orally, subcutaneously, topically, parenterally, by inhalation spray or rectally in dosage unit formulations containing conventional non-toxic pharmaceutically acceptable carriers, adjuvants and vehicles. Parenteral administration may include subcutaneous injections, intravenous or intramuscular injections or infusion techniques.

In some embodiments, the one or more additional therapeutic agent that may be administered in combination with a compound described herein (e.g., Formula II, Formula III, Formula IV, Formula V, Formula VI, Formula VII, Formula VIII, Formula IX, Formula X, Formula XI, Formula XII, Formula XIII, Formula XIV, Formula XV, Formula XVI, or Formula XVII, or a pharmaceutically acceptable salt, enantiomer, stereoisomer, or tautomer thereof) can be a RTK pathway inhibitor, a MAPKAP pathway inhibitor, a PI3K inhibitor, an mTOR inhibitor, an immunomodulatory agent, or a chemotherapeutic agent, or any combination thereof.

In some embodiments, the additional therapeutic agent is a RTK pathway inhibitor. Such RTK pathway inhibitors include, for example, KIT inhibitors, EGFR inhibitors, PDGFRα inhibitors, VEGFR inhibitors, anti-VEGF therapeutics, BCR-Abl inhibitors, and ALK inhibitors.

Exemplary KIT inhibitors include, but are not limited, to ripretinib, avaprinib, sunitinib, AZD3229, THE-630, and imatinib, and pharmaceutically acceptable salts thereof. Exemplary EGFR inhibitors include, but are not limited to, cetuximab, osimertinib, and afatinib, and pharmaceutically acceptable salts thereof.

Exemplary PDGFRα inhibitors include, but are not limited to, ripretinib, JNJ10198409, or a pharmaceutically acceptable salt thereof. Exemplary VEGFR inhibitors include, but are not limited to, regorafenib, axitinib, and pazopanib, and pharmaceutically acceptable salts thereof. Exemplary anti-VEGF therapeutics include bevacizumab. Exemplary BCR-Abl inhibitors include, but are not limited to, imatinib, nilotinib, dasatinib, or a pharmaceutically acceptable salt thereof. Exemplary ALK inhibitors include, but are not limited to, lorlatinib and alectinib, and pharmaceutically acceptable salts thereof.

In some embodiments, the additional therapeutic agent is a MAPKAP pathway inhibitor. Such MAPKAP pathway inhibitors include, for example, MEK inhibitors, ERK inhibitors, RAF inhibitors, and Ras inhibitors.

Exemplary MEK inhibitors include, but are not limited to, trametinib, selumetinib, cobimetinib, binimetinib, mirdametinib, and VS-6766, and pharmaceutically acceptable salts thereof.

Exemplary ERK inhibitors include, but are not limited to, ulixertinib, SCH772984, LY3214996, ravoxertinib, VX-11e, ERAS-007, and ASTX-029, and pharmaceutically acceptable salts thereof.

Exemplary RAF inhibitors include, but are not limited to, LY3009120, LXH254, RAF709, dabrafenib, vemurafenib, belvarafenib, KIN-2787, and VS-6766, and pharmaceutically acceptable salts thereof.

Exemplary Ras inhibitors include, but are not limited to, AMG-510, MRTX849, GDC-6036, MRTX-1133, RMC-9805, RMC-6291, and RMC-6236, and pharmaceutically acceptable salts thereof.

In some embodiments, the additional therapeutic agent is a PI3K inhibitor or an mTOR inhibitor.

Exemplary PI3K inhibitors include, but are not limited to, alpelisib, LY294002, and omipalisib, and pharmaceutically acceptable salts thereof.

Exemplary mTOR inhibitors include, but are not limited to, rapamycin, everolimus, PF04691502, and PP242, and pharmaceutically acceptable salts thereof.

The compounds described herein may be administered in combination with other therapeutic agents known to treat cancers. Such other therapeutic agents include radiation therapy, anti-tubulin agents, DNA alkylating agents, DNA synthesis-inhibiting agents, DNA intercalating agents, anti-estrogen agents, anti-androgens, steroids, anti-EGFR agents, kinase inhibitors, mTOR inhibitors, PI3 kinase inhibitors, cyclin-dependent kinase inhibitors, CD4/CD6 kinase inhibitors, topoisomerase inhibitors, Histone Deacetylase (HDAC) inhibitors, DNA methylation inhibitors, anti-HER2 agents, anti-angiogenic agents, proteasome inhibitors, PARP inhibitors, cell cycle regulating kinase inhibitors, thalidomide, lenalidomide, antibody-drug-conjugates (ADCs), immunotherapeutic agents including immunomodulating agents, targeted therapeutic agents, cancer vaccines, and CAR-T cell therapy.

In an embodiment, the additional therapeutic agents can be chemotherapeutic agents including but not limited to an anti-tubulin agents (for example, paclitaxel, paclitaxel protein-bound particles for injectable suspension including nab-paclitaxel, eribulin, docetaxel, ixabepilone, vincristine, auristatins, or maytansinoids), vinorelbine, DNA-alkylating agents (including cisplatin, carboplatin, oxaliplatin, cyclophosphamide, ifosfamide, temozolomide), DNA intercalating agents or DNA topoisomerase inhibitors (including anthracyclines such as doxorubicin, pegylated liposomal doxorubicin, daunorubicin, idarubicin, mitoxantrone, or epirubicin, camptothecins such as topotecan, irinotecan, or exatecan), 5-fluorouracil, capecitabine, cytarabine, decitabine, 5-aza cytadine, gemcitabine and methotrexate.

In some embodiments, the additional therapeutic agent is selected from the group consisting of anti-tubulin agents, vinorelbine, DNA-alkylating agents, DNA intercalating agents, 5-fluorouracil, capecitabine, cytarabine, decitabine, 5-azacytidine, gemcitabine, irinotecan, and methotrexate.

In some embodiments, the additional therapeutic agents can be kinase inhibitors including but not limited to erlotinib, gefitinib, neratinib, afatinib, osimertinib, lapatinib, crizotinib, brigatinib, ceritinib, alectinib, lorlatinib, everolimus, temsirolimus, abemaciclib, LEE011, palbociclib, cabozantinib, sunitinib, pazopanib, sorafenib, regorafenib, sunitinib, axitinib, dasatinib, imatinib, ripretinib, avapritinib, JNJ10198409, nilotinib, idelalisib, ibrutinib, BLU-285, BLU-667, Loxo 292, larotrectinib, and quizartinib, anti-estrogen agents including but not limited to tamoxifen, fulvestrant, anastrozole, letrozole, and exemestane, anti-androgen agents including but not limited to abiraterone acetate, enzalutamide, nilutamide, bicalutamide, flutamide, cyproterone acetate, steroid agents including but not limited to prednisone and dexamethasone, PARP inhibitors including but not limited to neraparib, olaparib, talazoparib, and rucaparib, topoisomerase I inhibitors including but not limited to irinotecan, camptothecin, exatecan, and topotecan, topoisomerase II inhibitors including but not limited to anthracyclines, etoposide, etoposide phosphate, and mitoxantrone, Histone Deacetylase (HDAC) inhibitors including but not limited to vorinostat, romidepsin, panobinostat, valproic acid, and belinostat, DNA methylation inhibitors including but not limited to DZNep and 5-aza-2′-deoxycytidine, proteasome inhibitors including but not limited to bortezomib and carfilzomib, thalidomide, lenalidomide, pomalidomide, biological agents including but not limited to trastuzumab, ado-trastuzumab, pertuzumab, cetuximab, panitumumab, ipilimumab, tremelimumab, anti-PD-1 agents including pembrolizumab, nivolumab, pidilizumab, and Cemiplimab, anti-PD-L1 agents including atezolizumab, avelumab, durvalumab and BMS-936559, anti-angiogenic agents including bevacizumab and aflibercept, and antibody-drug-conjugates (ADCs) including DM1, DM4, MMAE, MMAF, or camptothecin payloads, brentuximab vedotin and trastuzumab emtansine, radiotherapy, therapeutic vaccines including but not limited to sipuleucel-T.

In some embodiments, the additional therapeutic agents can be immunomodulatory agents including but not limited to anti-PD-1 or anti-PDL-1 therapeutics including pembrolizumab, nivolumab, atezolizumab, durvalumab, BMS-936559, avelumab, or dostarlimab, anti-TIM3 (anti-HAVcr2) therapeutics including but not limited to TSR-022 or MBG453, anti-LAG3 therapeutics including but not limited to relatlimab, LAG525, or TSR-033, anti-4-1BB (anti-CD37, anti-TNFRSF9), CD40 agonist therapeutics including but not limited to SGN-40, CP-870,893 or RO7009789, anti-CD47 therapeutics including but not limited to Hu5F9-G4, anti-CD20 therapeutics, anti-CD38 therapeutics, STING agonists including but not limited to ADU-S100, MK-1454, ASA404, or amidobenzimidazoles, anthracyclines including but not limited to doxorubicin or mitoxanthrone, hypomethylating agents including but not limited to azacytidine or decitabine, other immunomodulatory therapeutics including but not limited to epidermal growth factor inhibitors, statins, metformin, angiotensin receptor blockers, thalidomide, lenalidomide, pomalidomide, prednisone, or dexamethasone.

In some embodiments, the additional therapeutic agent is an immunomodulatory agent. In some embodiments, the immunomodulatory agent is selected from the group consisting of pembrolizumab, nivolumab, atezolizumab, durvalumab, BMS-936559, avelumab, cetuximab, TSR-022, MBG453, relatlimab, LAG525, TSR-033, SGN-40, CP-870,893, RO7009789, Hu5F9-G4, ADU-S100, MK-1454, ASA404, doxorubicin, mitoxanthrone, azacytidine, decitabine, statins, metformin, thalidomide, lenalidomide, pomalidomide, prednisone, dexamethasone, and dostarlimab, and pharmaceutically acceptable salts thereof.

In some embodiments, the additional therapeutic agent is selected from a luteinizing hormone-releasing hormone (LHRH) analog, including goserelin and leuprolide.

In some embodiments, the additional therapeutic agent is selected from the group consisting of selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatinib, nilotinib, dasatinib, panitumumab, amrubicin, oregovomab, Lep-etu, nolatrexed, azd2171, batabulin, of atumtunab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)-ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser(Bu t) 6, Azgly 10](pyro-Glu-His-Trp-Ser-Tyr-D-Ser(Bu t)-Leu-Arg-Pro-Azgly-NH₂ acetate [C₅₉H84N₁₈Oi₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutanide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl anilide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deoxyuridine, cytosine arabinoside, 6-mercaptopurine, deoxycoformycin, calcitriol, valrubicin, mithramycin, vinblastine, vinorelbine, topotecan, razoxane, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimetidine, trastuzumab, denileukin diftitox, gefitinib, bortezomib, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zoledronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritumomab tiuxetan, androgens, decitabine, hexamethylmelamine, bexarotene, tositumomab, arsenic trioxide, cortisone, etidronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, sspegfilgrastim, erythropoietin, epoetin alfa and darbepoetin alfa, ipilimumab, vemurafenib, MRTX849, MRTX1133, AMG510, GDC-6036, RMC-9805, RMC-6291, RMC-6236, belvarafenib, KIN-2787, ERAS-007, ASTX-029, and mixtures thereof.

### Pharmaceutical Compositions and Kits

Another aspect of this disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions may be used in the form of a pharmaceutical preparation, for example, in solid, semisolid, or liquid form, which contains one or more of the compounds described herein, as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound provided herein, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills, and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills, and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous, or organic solvents, or mixtures thereof, and powders. Liquid dosage forms for oral administration include pharmaceutically acceptable emulsions, microemulsions, solutions, suspensions, syrups, and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams, and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Pharmaceutical compositions of the present disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another embodiment, provided are enteral pharmaceutical formulations including a disclosed compound and an enteric material, and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5.

Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methylacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives described herein.

Advantageously, described herein are kits for use by a e.g., a consumer in need of treatment of cancer. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### EXAMPLES

The compounds described herein can be prepared in a number of ways based on the teachings contained herein and synthetic procedures known in the art. In the description of the synthetic methods described below it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

The following abbreviation are used in this disclosure and have the following definitions: “ADP” is adenosine diphosphate, “aq” is aqueous, “ATP” is adenosine triphosphate, “Ar” is argon gas, “Boc” is tert-butylcarbonate, “BSA” is bovine serum albumin, “conc” is concentrated, “DBU” is 1,8-diazabicyclo[5.4.0]undec-7-ene, “DCM” is dichloromethane, “DDQ’ is 2,3-dichloro-5,6-dicyano-para-benzoquinone, “DCE” is 1,2-dichloroethane, “DIEA” is N,N-diisopropylethylamine, “DMF” is N,N-dimethylformamide, “DMSO-d₆” is dimethylsulfoxide-deuterium, “EDC” is 1-ethyl-3-(3-dimethylaminopropyl)carbodiimide, “ESI” is electrospray ionization, “EtOAc” is ethyl acetate, “EtOH” is ethanol, “GST” is glutathione S-transferase, “h” is hour or hours, “H₂O” is water, “HFIP” is hexafluoroisopropanol, “IC₅₀” is half maximal inhibitory concentration, “HOBt” is Hydroxybenzotriazole, “KF” is potassium fluoride, “LDA’ is lithium diisopropylamide, “LiOH” is lithium hydroxide, “M” is molarity, “MeCN” is acetonitrile, “MeOH” is methanol, “MgSO₄” is magnesium sulfate, “MHz” is megahertz, “min” is minute or minutes, “MS” is mass spectrometry, “MTBE” is methyl tert-butyl ether, “m/z” is mass/charge number, “Na₂CO₃” is sodium carbonate, “NADH” is nicotinamide adenine dinucleotide, “NaHCO₃” is sodium bicarbonate, “Na₂SO₄” is sodium sulfate, “NMR” is nuclear magnetic resonance, “PBS” is phosphate buffered saline, “Pd” is palladium, “Pd(OAc)₂” is palladium (II) acetate, “PMB” is para-methoxybenzyl, “rt” is room temperature which is also known as “ambient temp,” which will be understood to consist of a range of normal laboratory temperatures ranging from 15-25° C., “sat'd.” is saturated, “SM” is starting material, “S_{N}” is nucleophilic substitution, “T₃P” is 1-propanephosphonic acid anhydride, “TBAI” is tetrabutylammonium iodide, “TEA” is triethylamine, “TFE” is trifluoroethanol, “THF” is tetrahydrofuran, “Xantphos” is 4,5-bis(diphenylphosphino)-9,9-dimethylxanthene, and “Yb(OTF)₃” is tris(((trifluoromethyl)sulfonyl)oxy)ytterbium.

### General Chemistry

Exemplary compounds described herein are available by the general synthetic methods illustrated in the Schemes below, intermediate preparations, and the accompanying Examples.

### Synthetic Schemes

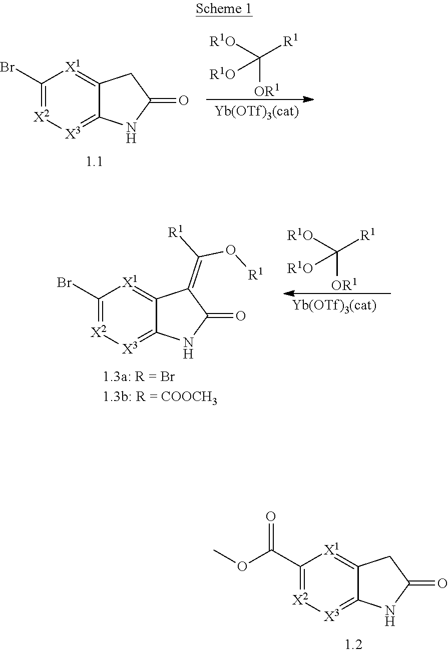

Scheme 1 illustrates an exemplary preparation of intermediates 1.3a and 1.3b. Both bromides 1.1 and esters 1.2 react with R¹C(OR¹)₃ under a Lewis acid condition such as tris(((trifluoromethyl)sulfonyl)oxy)ytterbium in an aprotic solvent at elevated temperature to obtain alkoxyalkylidenes 1.3a and 1.3b respectively.

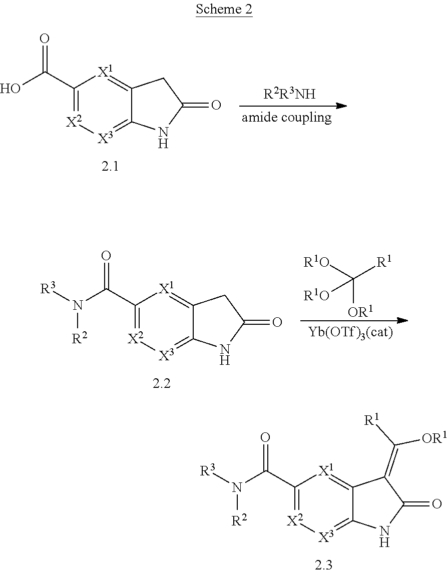

Scheme 2 illustrates an exemplary preparation of intermediates 2.3. Amide coupling reaction of 2.1 with amines R²R³NH under a typical amide coupling condition affords amides 2.2. Amides 2.2 react with R¹C(OR¹)₃ under a Lewis acid condition such as tris(((trifluoromethyl)sulfonyl)oxy)ytterbium in an aprotic solvent at elevated temperature to obtain alkoxyalkylidenes 2.3.

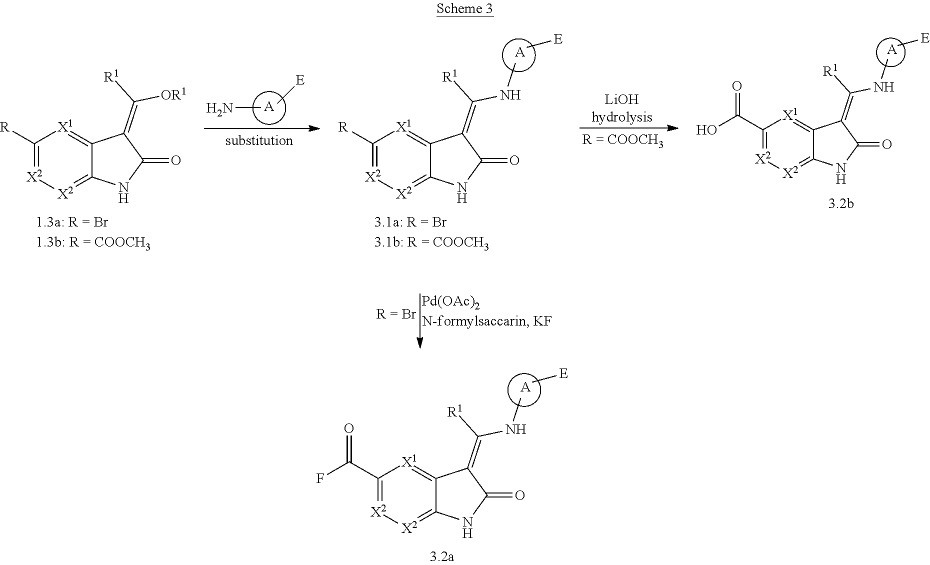

Scheme 3 illustrates an exemplary preparation of key intermediates 3.2a and 3.2b. Both bromides 1.3a and esters 1.3b react with amines E-A-NH₂ by substitution reaction in a protic solvent such as MeOH and EtOH at elevated temperature to obtain 3.1a and 3.1b respectively. Fluorocarbonylation of aryl bromides 3.1a (R═Br) using N-formylsaccarin and KF under Pd catalyst such as Pd(OAc)₂ in DMF at elevated temperature to afford acyl fluorides 3.2a (Org. Lett., 2013, 15, 5360-5373). Hydrolysis of 3.1b (R═COOCH₃) under basic conditions such as LiOH in polar solvents such as MeOH, water, and 1,4-dioxane at elevated temperature obtains the carboxylic acids 3.2b.

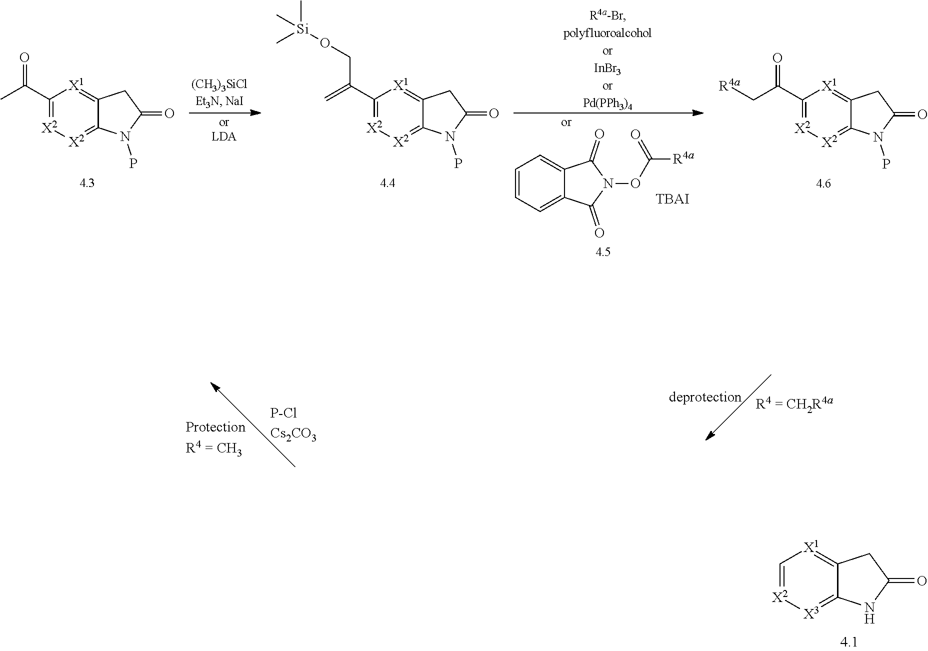

Scheme 4 illustrates an exemplary preparation of intermediates 4.7. Friedel-craft acylation of 4.1 with typical acylating agents R⁴COCl in the presence of Lewis acid catalyst such as AlCl₃, and FeCl₃ affords ketones 4.2. Ketones 4.2 react with P—Cl in the presence of a base such as Cs₂CO₃ in DMF to afford P-protected (P can be benzyl or PMB group) compounds 4.3. Silyl enolation of compounds 4.3 with trimethylchlorosilane under sodium iodide-tertiary amine or LDA conditions in an aprotic solvent such as acetonitrile, and DCM affords enolates 4.4. Compounds 4.6 can be prepared by (1) alkylation of silyl enol ethers 4.4 with R^{4a}Br (alpha-keto bromides) under a polyfluoroalcohol (TFE and HFIP) as a solvent without catalyst (CN104844401), (2) indium-catalyzed coupling reaction between enol ethers 4.4 and alkylating reagents alkyl chloride R^{4a}Cl or alkyl ethers R^{4a}—O—R^{4a} (Tetrahedron, 2009, 65, 5462-5471), (3) Pd-catalyzed dual ligand-enable alkylation of silyl enol ethers 4.4 with alkylating reagent R^{4a}Br (ACS catalyst, 2020, 10, 1334-1343), and (4) photocatalytic decarboxylative alkylation of silyl enol ethers 4.4 with reagents 4.5 using TBAI (Org. Chem. Frontiers, 2021, 8, 4166-4170). Compounds 4.6 can be deprotected under strongly acidic conditions (TFA with trifluoromethanesulfonic acid) or mildly oxidizing conditions using DDQ to produce compounds 4.2 (R⁴═CH₂R^{4a}). Finally, ketones 4.2 react with R¹C(OR¹)₃ under a Lewis acid condition such as tris(((trifluoromethyl)sulfonyl)oxy)ytterbium in an aprotic solvent at elevated temperature to obtain alkoxyalkylidenes 4.7.

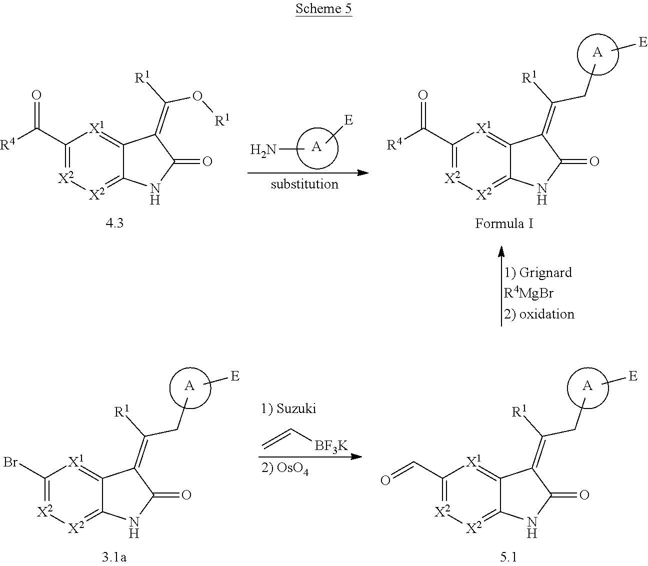

Scheme 5 illustrates an exemplary preparation of compounds of Formula I. Key intermediates, aldehydes 5.1 can be prepared from bromides 3.1a by reaction with potassium vinyltrifluoroborate in the presence of a palladium catalyst (Suzuki reaction), followed by oxidation with OsO₄ and sodium periodate. Aldehydes 5.1 react with Grignard reagents R⁴MgBr to provide the secondary alcohols and then the alcohols can be oxidized using Dess-Martin periodinane in aprotic solvent to afford compounds of Formula I. Alternatively, compounds of Formula I can be prepared by substitution reaction of 4.3 with amines E-A-NH₂.

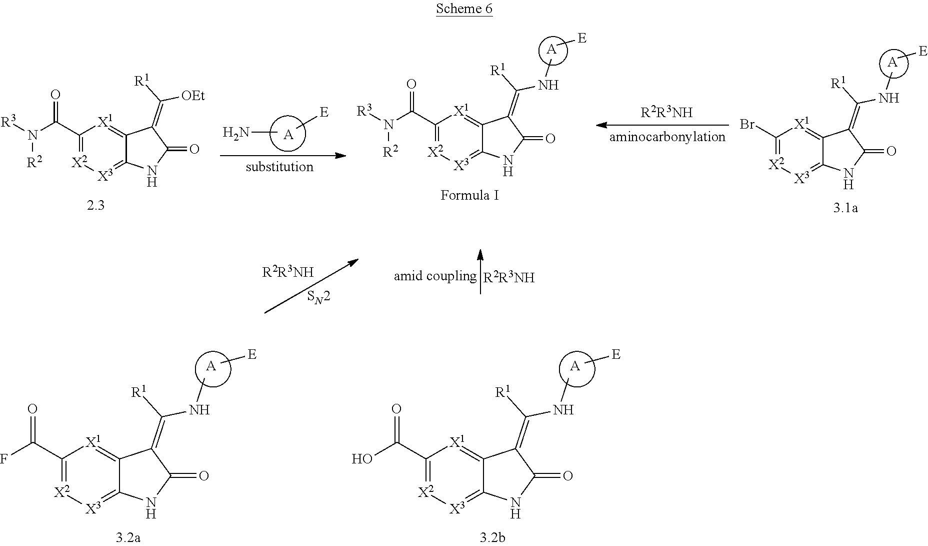

Scheme 6 illustrates an exemplary preparation of compounds of Formula I. Compounds of Formula I can be prepared substitution reaction of 2.3 with amines E-A-NH₂. Pd-catalyzed aminocarbonylation of 3.1a with amines (R²R³NH) using either Mo(CO)₆ or Co₂(CO)₈ as CO source to afford compounds of Formula I (Tetrahedron Lett., 2013, 54, 6912-6915 and J. Org. Chem., 2021, 86, 5530-5537). Alternatively, compounds of Formula I can be prepared from acyl fluoride 3.2a with amines (R²R³NH) under substitution reaction. In another embodiment, acids 3.2b react with amines (R²R³NH) in the presence of amide coupling reagents such as EDC, T3P and HATU in an aprotic solvent such as DCM to afford compounds of Formula I.

### Preparation of Intermediates

Using the synthetic procedures and methods described herein and methods known to those skilled in the art, the following compounds were made:

General Method A: Amide formation

### Example A1: 2-oxo-N-(2,2,2-trifluoroethyl)indoline-5-carboxamide

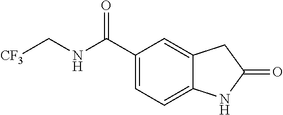 [SMILES: O=C1Cc2cc(C(=O)NCC(F)(F)F)ccc2N1]

A suspension of 2-oxoindoline-5-carboxylic acid (5.0 g, 28 mmol) in DCM (150 mL) was treated with HOBt (4.5 g, 30 mmol) and EDC (5.7 g, 30 mmol). The reaction was stirred at rt for 15 min then 2,2,2-trifluoroethan-1-amine (3.4 g, 34 mmol) was added. The reaction mixture stirred at rt for 2 h. DIEA (4.9 mL, 28 mmol) was added and the reaction was stirred at rt for 2 h. The reaction was diluted with EtOAc and the organic layer was washed with aq 1.0 M HCl (50 mL), then sat'd NaHCO₃ solution (3×). The organic layer was dried over anhydrous Na₂SO₄, filtered, and concentrated under reduced pressure to obtain 2-oxo-N-(2,2,2-trifluoroethyl)indoline-5-carboxamide (4.5 g, 62%). ¹H NMR (500 MHz, DMSO-d₆): δ 10.67 (s, 1H), 8.90 (t, J=6.4 Hz, 1H), 7.86 (s, 1H), 7.84 (s, 1H), 6.88 (d, J=8.0 Hz, 1H), 4.01-4.10 (m, 2H), 3.54 (s, 2H); MS (ESI) m/z: 259.0 (M+H⁺).

### General Method B: Carbonylation

### Example A2: N-isobutyl-2-oxoindoline-5-carboxamide

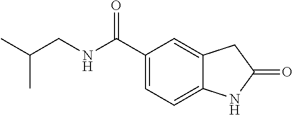 [SMILES: CC(C)CNC(=O)c1ccc2c(c1)CC(=O)N2]

A solution of 5-bromoindolin-2-one (1.5 g, 7.1 mmol) in 1,4-dioxane (15 mL) was treated with Na₂CO₃ (2.2 g, 21 mmol) at rt and then the solution was purging with nitrogen for 5 min. Mo(CO)₆ (0.93 g, 3.5 mmol) and Herrmann-Beller catalyst (0.33 g, 0.35 mmol) were added and continued nitrogen purging for another 5 min. 2-Methylpropan-1-amine (1.6 g, 21 mmol) was added and the reaction mixture was heated at 145° C. for 16 h. The reaction was diluted with DCM and filtered through a pad of celite. The filtrate was washed with sat'd NaHCO₃ and the organic layer was concentrated under reduced pressure. The crude material was purified via silica gel column chromatography (MeOH/DCM) to obtain N-isobutyl-2-oxo-2,3-dihydro-1H-pyrrolo[2,3-b]pyridine-5-carboxamide (0.48 g, 29%) as an off white solid. ¹H NMR (500 MHz, DMSO-d₆): δ 10.52 (s, 1H), 8.20 (t, J=5.2 Hz, 1H), 7.71 (m, 2H), 6.82 (d, J=8.8 Hz, 1H), 3.51 (s, 2H), 3.05 (t, J=6.8 Hz, 2H), 1.82 (m, 1H), 0.86 (d, J=6.4 Hz, 6H); MS (ESI) m/z: 233.2 (M+H⁺).

### General Method C: Friedel-Crafts Acylation

### Example A3: 5-acetylindolin-2-one

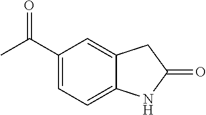

An RBF was charged with AlCl₃ (2.5 g, 19 mmol) and DCE (8 mL) and then the solution was cooled to 0° C. under an ice-water bath. Oxindole (1.0 g, 7.7 mmol) was added and acetyl chloride (1.1 mL, 15 mmol) was added dropwise over 5 min. The reaction was allowed to gradually warm to rt and then stirred at rt overnight. The reaction mixture was carefully added to ice (300 mL) and the slurry was stirred until the ice melted. The solids were filtered, washed with water, and dried in the vacuum oven for 2 h to obtain 5-acetylindolin-2-one (1.15 g, 86%) as a tan solid. ¹H NMR (500 MHz, DMSO-d₆): δ 10.74 (s, 1H), 7.85 (dd, J 8.2, 1.7 Hz, 1H), 7.79 (s, 1H), 6.89 (d, J 8.2 Hz, 1H), 3.54 (s, 2H), 2.49 (s, 3H).

Using the General Methods A, B and C above, the following Intermediates of Table A were prepared.

TABLE A

| Ex | | | | Yield | ¹H NMR (400 or 500 | MS (m/z: |
|---|---|---|---|---|---|---|
| No | SM | Product | Method | (%) | MHz, DMSO-d₆): δ | M + H⁺) |
| A4 | 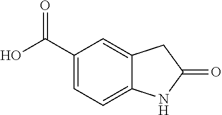 | 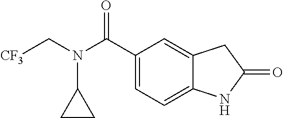 [SMILES: O=C1Cc2cc(C(=O)N(CC(F)(F)F)C3CC3)ccc2N1] | A | 25 | 10.61 (s, 1H), 7.41 (d, J = 6.4 Hz , 2H), 6.83 (d, J = 8.8 Hz , 1H), 4.31 (q, J = 8.0 Hz, 2H), 3.52 (s, 2H), 2.94 (m, 1H), 0.62 (m, 2H), 0.48 (m, 2H). | 298.9 |
| | 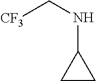 | | | | | |
| A5 | 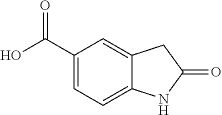 | 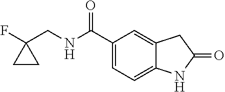 [SMILES: O=C1Cc2cc(C(=O)NCC3(F)CC3)ccc2N1] | A | 44 | No Data | 249.2 |
| | 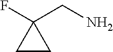 | | | | | |
| A6 | 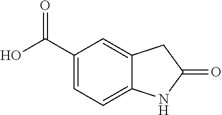 | 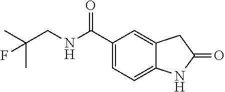 [SMILES: CC(C)(F)CNC(=O)c1ccc2c(c1)CC(=O)N2] | A | crude | 10.64 (s, 1H), 8.42 (t, J = 6.2 Hz, 1H), 7.74- 7.80 (m, 2H), 6.85 (d, J = 6.5 Hz, 1H), 3.53 (s, 2H), 3.45 (dd, J = 19.5, 6.0 Hz, 2H), 1.30 (d, J = 21.5 Hz, 6H). | 251.2 |
| | 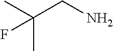 | | | | | |
| A7 | 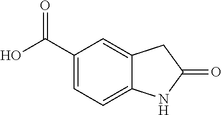 | 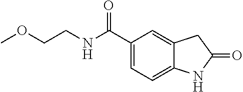 [SMILES: COCCNC(=O)c1ccc2c(c1)CC(=O)N2] | A | 49 | No Data | 257.0 (M + Na)⁺ |
| | 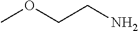 | | | | | |
| A8 | 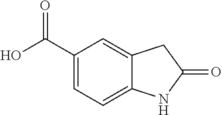 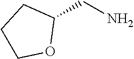 | 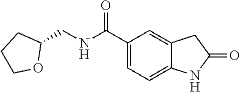 [SMILES: O=C1Cc2cc(C(=O)NC[C@H]3CCCO3)ccc2N1] | A (T3P) | 61 | 10.61 (s, 1H), 8.35 (d, J = 5.9 Hz, 1H), 7.72 (m, 2H), 6.83 (d, J = 8.0 Hz, 1H), 3.94 (m, 1H), 3.75 (m, 1H), 3.60 (m, 1H), 3.52 (s, 2H), 3.20-3.33 (m, 2H), 1.71-1.93 (m, 3H), 1.56 (m, 1H). | 261.2 |
| A9 | 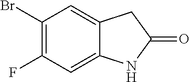 | 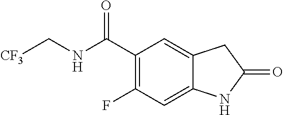 [SMILES: O=C1Cc2cc(C(=O)NCC(F)(F)F)c(F)cc2N1] | B | 32 | 10.76 (s, 1H), 8.67 (br s, 1H), 7.48 (d, J = 6.8 Hz, 1H), 6.67 (d, J = 11.2 Hz, 1H), 4.05 (m, 2H), 3.50 (s, 2H). | 275.1 (negative) |
| | 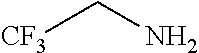 | | | | | |
| A10 | 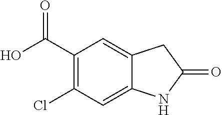 | 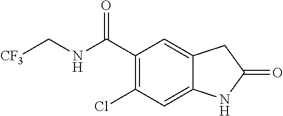 [SMILES: O=C1Cc2cc(C(=O)NCC(F)(F)F)c(Cl)cc2N1] | A | 53 | No Data | 293.0 |
| | 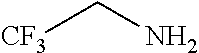 | | | | | |
| A11 | 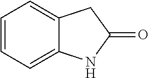 | 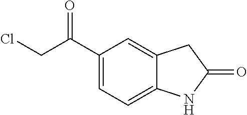 [SMILES: O=C1Cc2cc(C(=O)CCl)ccc2N1] | C | 90 | 10.83 (s, 1H), 7.86 (d, J = 8.4 Hz, 1H), 7.82 (s, 1H), 6.91 (d, J = 8.4 Hz, 1H), 5.07 (s, 2H), 3.56 (s, 2H). | 208.0 (negative) |
| | 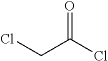 | | | | | |
| A12 | 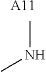 | 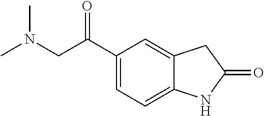 [SMILES: CN(C)CC(=O)c1ccc2c(c1)CC(=O)N2] | S_{N} | 30 | 10.74 (s, 1H), 7.88 (d, J = 8.4 Hz, 1H), 7.83 (s, 1H), 6.87 (d, J = 8.4 Hz, 1H), 3.62 (s, 2H), 3.54 (s, 2H), 2.21 (s, 6H). | 218.9 (negative) |
| A13 | 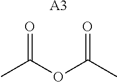 | 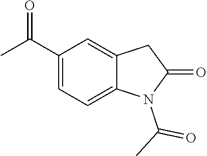 [SMILES: CC(=O)c1ccc2c(c1)CC(=O)N2C(C)=O] | acylation | crude | No Data | 218.2 |

### General Method D: Formation of Enol-Ether Under a Lewis Acid

### Example B1: (Z)-3-(1-ethoxypropylidene)-2-oxo-N-(2,2,2-trifluoroethyl)indoline-5-carboxamide

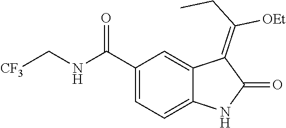 [SMILES: CCO/C(CC)=C1\C(=O)Nc2ccc(C(=O)NCC(F)(F)F)cc21]

A suspension of 2-oxo-N-(2,2,2-trifluoroethyl)indoline-5-carboxamide (A1, 4.5 g, 17 mmol) and 1,1,1-triethoxypropane (7.0 mL, 35 mmol) in DCE (80 mL) was treated with Yb(OTf)₃ (0.54 g, 0.87 mmol). The reaction mixture was heated to 80° C. overnight. The reaction was cooled to rt and concentrated under reduced pressure. The crude was suspended in MeCN and the solids were collected via vacuum filtration to obtain (Z)-3-(1-ethoxypropylidene)-2-oxo-N-(2,2,2-trifluoroethyl)indoline-5-carboxamide (4.4 g, 73%) as a light mauve solid. MS (ESI) m/z: 343.2 (M+H⁺).

Using the General Method D above, the following Intermediates of Table B were prepared.

TABLE B

| Ex | | | Yield | ¹H NMR (400 or 500 MHz, DMSO- | MS (m/z: |
|---|---|---|---|---|---|
| No | SM | Product | (%) | d₆): δ | M + H⁺) |
| B2 | 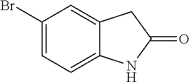 | 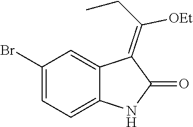 [SMILES: CCO/C(CC)=C1\C(=O)Nc2ccc(Br)cc21] | 80 | 10.23 (s, 1H), 7.56 (d, J = 2.0 Hz, 1H), 7.08 (dd, J = 8.2, 2.1 Hz, 1H), 6.59 (d, J = 8.2 Hz, 1H), 4.26 (q, J = 7.0 Hz, 2H), 3.04 (q, J = 7.4 Hz, 2H), 1.28 (t, J = 7.0 Hz, 3H), 1.01 (t, J = 7.5 Hz, 3H). | 296.0 298.0 |
| B3 | B2 (Boc)₂O | 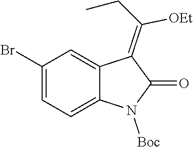 [SMILES: CCO/C(CC)=C1\C(=O)N(C(=O)OC(C)(C)C)c2ccc(Br)cc21] | 40 | 7.88 (s, 1H), 7.65 (d, J = 8.4 Hz, 1H), 7.36 (d, J = 8.8 Hz, 1H), 4.49 (q, J = 6.8 Hz, 2H), 3.15 (q, J = 7.6 Hz, 2H), 1.56 (s, 9H), 1.45 (t, J = 6.8 Hz, 3H), 1.17 (t, J = 7.2 Hz, 3H). | No Data |
| B4 | 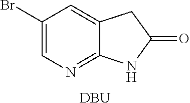 | 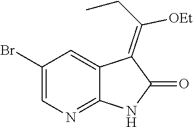 [SMILES: CCO/C(CC)=C1\C(=O)Nc2ncc(Br)cc21] | crude | No Data | 297.0 299.0 |
| B5 | 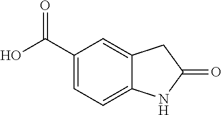 | 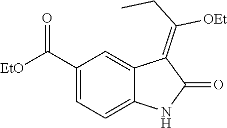 [SMILES: CCOC(=O)c1ccc2c(c1)/C(=C(\CC)OCC)C(=O)N2] | 59 | 10.62 (s, 1H), 8.30 (d, J = 1.7 Hz, 1H), 7.73 (dd, J = 8.1, 1.7 Hz, 1H), 6.86 (d, J = 8.1 Hz, 1H), 4.41 (q, J = 6.9 Hz, 2H), 4.25 (q, J = 7.1 Hz, 2H), 3.18 (q, J = 7.4 Hz, 2H), 1.46 (t, J = 7.0 Hz, 3H), 1.31 (t, J = 7.1 Hz, 3H), 1.16 (t, J = 7.4 Hz, 3H). | 290.2 |
| B6 | A1 | 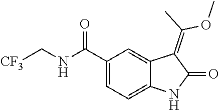 [SMILES: CO/C(C)=C1\C(=O)Nc2ccc(C(=O)NCC(F)(F)F)cc21] | 60 | 10.53 (s, 1H), 8.85 (t, J = 6.4 Hz, 1H), 8.12 (s, 1H), 7.66 (t, J = 1.2 Hz, 1H), 6.82 (d, J = 8.0 Hz, 1H), 4.06 (m, 5H), 2.74 (s, 3H). | 315 |
| B7 | A2 | 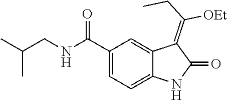 [SMILES: CCO/C(CC)=C1\C(=O)Nc2ccc(C(=O)NCC(C)C)cc21] | 70 | 10.44 (s, 1H), 8.21 (br t, J = 4.8 Hz, 1H), 8.12 (d, J = 1.8 Hz, 1H), 7.55 (dd, J = 8.1, 1.8 Hz, 1H), 6.79 (d, J = 8.1 Hz, 1H), 4.39 (q, J = 7.0 Hz, 2H), 3.19 (q, J = 7.4 Hz, 2H), 3.05 (t, J = 6.4 Hz, 2H), 1.82 (m, 1H), 1.45 (t, J = 7.0 Hz, 3H), 1.16 (t, J = 7.4 Hz, 3H), 0.89 (d, J = 6.7 Hz, 6H). | 317.2 |
| B8 | A3 | 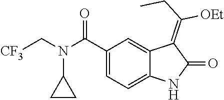 [SMILES: CCO/C(CC)=C1\C(=O)Nc2ccc(C(=O)N(CC(F)(F)F)C3CC3)cc21] | 43 | No Data | 383.1 |
| B9 | A4 | 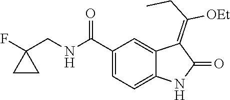 [SMILES: CCO/C(CC)=C1\C(=O)Nc2ccc(C(=O)NCC3(F)CC3)cc21] | crude | No Data | 333.2 |
| B10 | A5 | 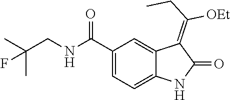 [SMILES: CCO/C(CC)=C1\C(=O)Nc2ccc(C(=O)NCC(C)(C)F)cc21] | 38 | 10.47 (s, 1H), 8.33 (br t, J = 6.3 Hz, 1H), 8.15 (s, 1H), 7.60 (d, J = 8.0 Hz, 1H), 6.80 (d, J = 8.1 Hz, 1H), 4.39 (q, J = 6.9 Hz, 2H), 3.45 (dd, J = 19.3, 6.2 Hz, 2H), 3.19 (q, J = 7.5 Hz, 2H), 1.45 (t, J = 6.9 Hz, 3H), 1.33 (d, J = 21.5 Hz, 6H), 1.16 (t, J = 7.5 Hz, 3H). | 357.2 (M + Na)⁺ |
| B11 | A6 | 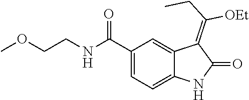 [SMILES: CCO/C(CC)=C1\C(=O)Nc2ccc(C(=O)NCCOC)cc21] | 12 | No Data | 341.2 (M + Na)⁺ |
| B12 | A7 | 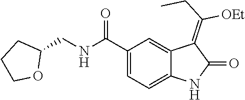 [SMILES: CCO/C(CC)=C1\C(=O)Nc2ccc(C(=O)NC[C@H]3CCCO3)cc21] | 65 | 10.47 (s, 1H), 8.25 (br s, 1H), 8.12 (s, 1H), 7.56 (dd, J = 8.0, 1.6 Hz, 1H), 6.78 (d, J = 8.0 Hz, 1H), 4.39 (q, J = 6.9 Hz, 2H), 3.94 (m, 1H), 3.76 (q, J = 7.0 Hz, 1H), 3.60 (q, J = 7.3 Hz, 1H), 3.20-3.32 (m, 2H), 3.17 (q, J = 7.4 Hz, 2H), 1.72-1.93 (m, | 345.2 |
| | | | | 3H), 1.58 (m, 1H), 1.44 (t, J = 6.9 | |
| | | | | Hz, 3H), 1.14 (t, J = 7.4 Hz, 3H). | |
| B13 | A8 | 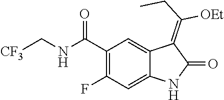 [SMILES: CCO/C(CC)=C1\C(=O)Nc2cc(F)c(C(=O)NCC(F)(F)F)cc21] | 28 | 10.64 (s, 1H), 8.65 (br s, 1H), 7.92 (d, J = 7.2 Hz, 1H), 6.63 (d, J = 11.2 Hz, 1H), 4.39 (q, J = 6.8 Hz, 2H), 3.90-4.09 (m, 2H), 3.16 (q, J = 7.6 Hz, 2H), 1.41 (t, J = 6.8 Hz, 3H), 1.15 (t, J = 7.2 Hz, 3H). | 361.1 |
| B14 | A9 | 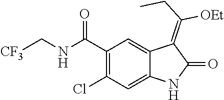 [SMILES: CCO/C(CC)=C1\C(=O)Nc2cc(Cl)c(C(=O)NCC(F)(F)F)cc21] | 85 | No Data | 377.0 |
| B15 | A3 | 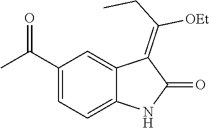 [SMILES: CCO/C(CC)=C1\C(=O)Nc2ccc(C(C)=O)cc21] | 74 | 10.62 (s, 1H), 8.25 (d, J = 1.7 Hz, 1H), 7.74 (dd, J = 8.2, 1.8 Hz, 1H), 6.85 (d, J = 8.1 Hz, 1H), 4.41 (q, J = 7.0 Hz, 2H), 3.18 (q, J = 7.5 Hz, 2H), 2.50 (s, 3H), 1.46 (t, J = 7.0 Hz, 3H), 1.16 (t, J = 7.5 Hz, 3H). | 260.2 |
| B16 | A12 | 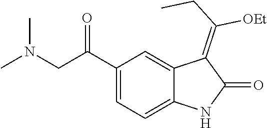 [SMILES: CCO/C(CC)=C1\C(=O)Nc2ccc(C(=O)CN(C)C)cc21] | 20 | 10.64 (s, 1H), 8.32 (d, J = 1.2 Hz, 1H), 7.78 (m, 1H), 6.84 (d, J = 8.4 Hz, 1H), 4.39 (m, 2H), 3.64 (s, 2H), 3.19 (t, J = 7.2 Hz, 2H), 2.23 (s, 6H), 1.47 (t, J = 6.8 Hz, 3H), 1.16 (t, J = 7.6 Hz, 3H). | 303.0 |
| B17 | A13 DBU | 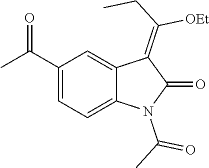 [SMILES: CCO/C(CC)=C1\C(=O)N(C(C)=O)c2ccc(C(C)=O)cc21] | 53 | 8.40 (d, J = 1.9 Hz, 1H), 8.19 (d, J = 8.5 Hz, 1H), 7.85 (dd, J = 8.5, 1.9 Hz, 1H), 4.52 (q, J = 7.0 Hz, 2H), 3.20 (q, J = 7.5 Hz, 2H), 2.63 (s, 3H), 2.56 (s, 3H), 1.51 (t, J = 7.0 Hz, 3H), 1.21 (t, J = 7.5 Hz, 3H). | 302.2 |

### General Method E: Substitution Reaction

### Example C1: ethyl (Z)-3-(1-((1-isopropyl-1H-pyrazol-4-yl)amino)propylidene)-2-oxoindoline-5-carboxylate

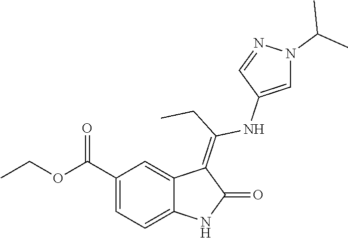 [SMILES: CCOC(=O)c1ccc2c(c1)/C(=C(\CC)Nc1cnn(C(C)C)c1)C(=O)N2]

A solution of ethyl (Z)-3-(1-ethoxypropylidene)-2-oxoindoline-5-carboxylate (B5, 5.0 g, 17 mmol) and 1-isopropyl-1H-pyrazol-4-amine (2.2 g, 17 mmol) in EtOH (50 ml) was heated to 90° C. overnight. The reaction was cooled to rt and concentrated under reduced pressure. The residue was treated with MTBE and sonicated for 15 min. The solids were then filtered to obtain ethyl (Z)-3-(1-((1-isopropyl-1H-pyrazol-4-yl)amino)propylidene)-2-oxoindoline-5-carboxylate (5.6 g, 87%) as a brown solid. ¹H NMR (500 MHz, DMSO-d₆): δ 11.69 (s, 1H), 10.96 (s, 1H), 8.01 (s, 1H), 7.86 (d, J=1.6 Hz, 1H), 7.68 (dd, J=8.2, 1.5 Hz, 1H), 7.56 (s, 1H), 6.98 (d, J=8.2 Hz, 1H), 4.50 (m, 1H), 4.28 (q, J 7.1 Hz, 2H), 2.77 (q, J=7.6 Hz, 2H), 1.44 (d, J 6.7 Hz, 6H), 1.23-1.34 ((J, 6H); MS (ESI) m/z: 369.2 (M+H⁺).

Using the General Method E above, the following Intermediates of Table C were prepared.

TABLE C

| Ex | | | Yield | ¹H NMR (400 or 500 MHz, | MS (m/z: |
|---|---|---|---|---|---|
| No | SM | Product | (%) | DMSO-d₆): δ | M + H⁺) |
| C2 | 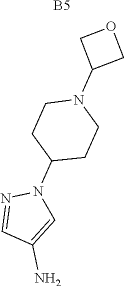 | 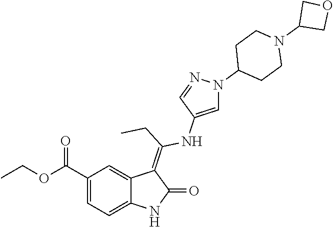 | 58 | 11.69 (s, 1H), 10.96 (s, 1H), 8.05 (s, 1H), 7.86 (d, J = 1.6 Hz, 1H), 7.68 (dd, J = 8.2, 1.5 Hz, 1H), 7.59 (d, J = 0.8 Hz, 1H), 6.98 (d, J = 8.1 Hz, 1H), 4.55 (t, J = 6.5 Hz, 2H), 4.44 (t, J = 6.1 Hz, 2H), 4.28 (q, J = 7.1 Hz, 2H), 4.17 (m, 1H), 3.44 (m, 1H), 2.74-2.80 (m, 4H), 1.90-2.05 (m, 6H), 1.23-1.34 (m, 6H). | 466.2 |
| C3 | 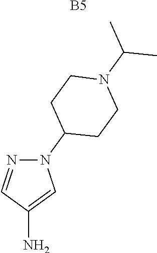 | 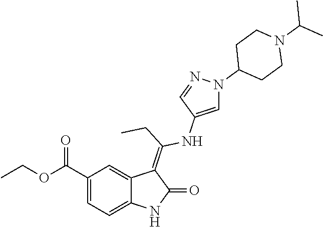 | 78 | No Data | 452.2 |
| C4 | 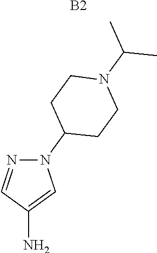 | 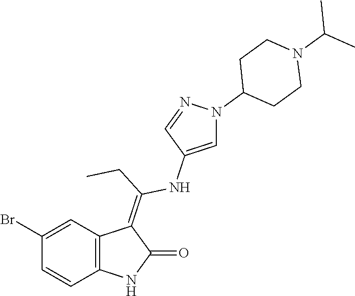 | 63 | 11.76 (s, 1H), 10.68 (s, 1H), 8.02 (s, 1H), 7.55 (s, 1H), 7.32 (s, 1H), 7.11 (d, J = 7.6 Hz, 1H), 6.82 (d, J = 8.4 Hz, 1H), 4.10 (br s, 1H), 2.86 (m, 2H), 2.70 (q, J = 7.2 Hz, 2H), 2.25 (m, 3H), 1.93 (m, 2H), 1.88 (m, 2H), 1.21 (t, J = 7.6 Hz, 3H), 0.97 (d, J = 6.4 Hz, 6H). | 458.2 460.2 |
| C5 | 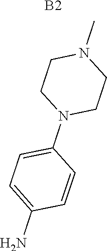 | 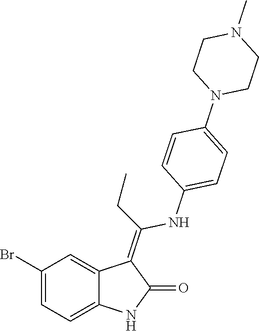 [SMILES: CC/C(Nc1ccc(N2CCN(C)CC2)cc1)=C1/C(=O)Nc2ccc(Br)cc21] | 76 | 12.03 (s, 1H), 10.67 (s, 1H), 7.30 (d, J = 1.8 Hz, 1H), 7.15 (d, J = 8.6 Hz, 2H), 7.11 (dd, J = 8.2, 1.8 Hz, 1H), 7.00 (d, J = 8.6 Hz, 2H), 6.83 (d, J = 8.2 Hz, 1H), 3.17 (m, 4H), 2.65 (q, J = 7.2 Hz, 2H), 2.45 (m, 4H), 2.22 (s, 3H), 1.18 (t, J = 7.4 Hz, 3H). | 441.2 443.2 |
| C6 | 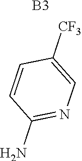 | 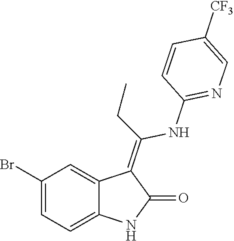 [SMILES: CC/C(Nc1ccc(C(F)(F)F)cn1)=C1/C(=O)Nc2ccc(Br)cc21] | 32 | 12.96 (s, 1H), 11.01 (s, 1H), 8.75 (s, 1H), 8.12 (d, J = 8.8 Hz, 1H), 7.51 (s, 1H), 7.26 (m, 2H), 6.88 (d, J = 8.4 Hz, 1H), 3.38 (q, J = 7.6 Hz, 2H), 1.39 (t, J = 7.2 Hz, 3H). | 409.9 411.9 (negative) |
| C7 | 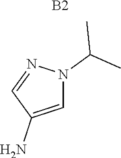 | 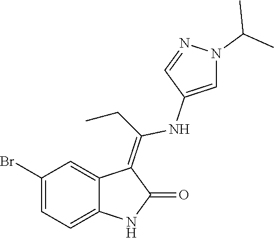 [SMILES: CC/C(Nc1cnn(C(C)C)c1)=C1/C(=O)Nc2ccc(Br)cc21] | 75 | 11.78 (s, 1H), 10.69 (s, 1H), 8.00 (s, 1H), 7.54 (s, 1H), 7.32 (d, J = 1.2 Hz, 1H), 7.13 (dd, J = 8.4 Hz, 1.6 Hz, 1H), 6.83 (d, J = 8.0 Hz, 1H), 4.48 (m, 1H), 2.70 (q, J = 7.2 Hz, 2H), 1.42 (d, J = 6.8 Hz, 6H), 1.21 (t, J = 7.6 Hz, 3H). | 374.9 |
| C8 | 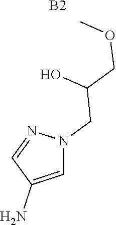 | 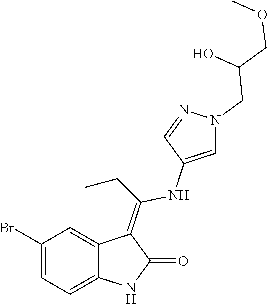 [SMILES: CC/C(Nc1cnn(CC(O)COC)c1)=C1/C(=O)Nc2ccc(Br)cc21] | 37 | No Data | 420.9 422.9 |
| C9 | 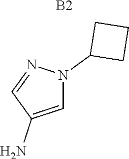 | 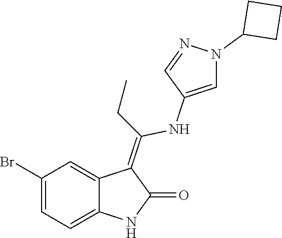 [SMILES: CC/C(Nc1cnn(C2CCC2)c1)=C1/C(=O)Nc2ccc(Br)cc21] | 42 | 11.77 (s, 1H), 10.70 (s,1H), 8.06 (s,1H), 7.58 (s, 1H), 7.32 (d, J = 1.6 Hz, 1H), 7.13 (dd, J = 8.4, 1.6 Hz, 1H), 6.83 (d, J = 8.4 Hz, 1H), 4.83 (m, 1H), 2.52-2.72 (m, 2H), 2.32- 2.49 (m, 2H), 1.75-1.82 (m, 2H), 1.21 (t, J = 7.6 Hz, 3H), 2Hs are under solvent peaks. | 387.0 389.0 |
| C10 | 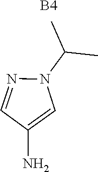 | 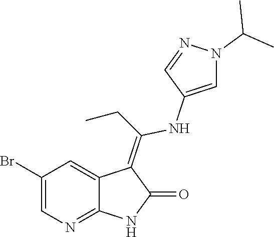 [SMILES: CC/C(Nc1cnn(C(C)C)c1)=C1/C(=O)Nc2ncc(Br)cc21] | 42 | No Data | 376.0 378.0 |
| C11 | 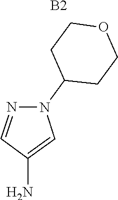 | 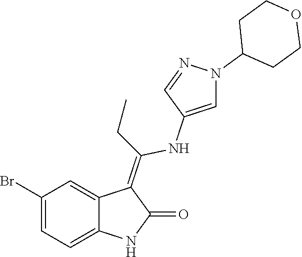 [SMILES: CC/C(Nc1cnn(C2CCOCC2)c1)=C1/C(=O)Nc2ccc(Br)cc21] | crude | 11.78 (s,1H), 10.70 (s, 1H), 8.05 (s, 1H), 7.58 (s, 1H), 7.33 (s, 1H), 7.12 (d, J = 8.0 Hz, 1H), 6.83 (d, J = 8.0 Hz, 1H), 4.40 (m, 1H), 3.94 (m, 2H), 3.46 (m, 2H), 2.71 (q, J = 7.6 Hz, 2H), 1.91-1.98 (m, 4H), 1.21 (t, J = 7.6 Hz, 3H). | 417.0 419.0 |
| C12 | 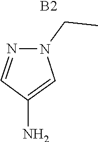 | 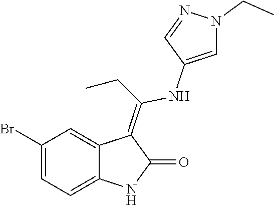 [SMILES: CC/C(Nc1cnn(CC)c1)=C1/C(=O)Nc2ccc(Br)cc21] | 68 | 11.77 (s, 1H), 10.69 (s, 1H), 7.97 (s, 1H), 7.54 (s, 1H), 7.32 (d, J = 1.2 Hz, 1H), 7.13 (dd, J = 8.4, 1.6 Hz, 1H), 6.83 (d, J = 8.4 Hz 1H), 4.15 (q, J = 7.2 Hz, 2H), 2.71 (q, J = 7.6 Hz, 2H), 1.38 (t, J = 7.2 Hz, 3H), 1.21 (t, J = 7.2 Hz, 3H). | 361.0 |
| C13 | 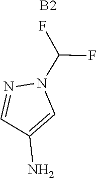 | 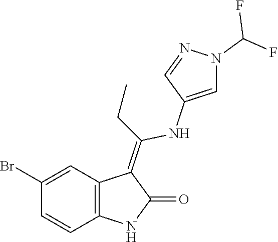 [SMILES: CC/C(Nc1cnn(C(F)F)c1)=C1/C(=O)Nc2ccc(Br)cc21] | 62 | 11.80 (s, 1H), 10.76 (s, 1H), 8.50 (s, 1H), 7.97 (s, 1H), 7.36 (s, 1H), 7.15 (d, J = 8.8 Hz, 1H), 6.84 (d, J = 8.4 Hz, 1H), 7.68 (m, 1H), 2.70 (q, J = 7.2 Hz, 2H), 1.21 (t, J = 7.6 Hz, 3H). | 382.9 |
| C14 | 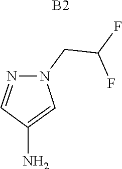 | 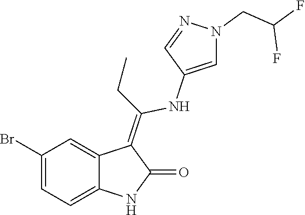 [SMILES: CC/C(Nc1cnn(CC(F)F)c1)=C1/C(=O)Nc2ccc(Br)cc21] | 62 | 11.78 (s, 1H), 10.71 (s, 1H), 8.03 (s, 1H), 7.67 (s, 1H), 7.34 (d, J = 1.2 Hz, 1H), 7.14 (d, J = 1.6 Hz, 1H), 6.83 (d, J = 8.4 Hz, 1H), 6.26-6.52 (m, 1H), 4.60-4.68 (m, 2H), 2.68- 2.73 (m, 2H), 1.21 (t, J = 7.6 Hz, 3H). | 397.0 |

### General Method F: Hydrolysis

### Example D1: (Z)-3-(1-((1-isopropyl-1H-pyrazol-4-yl)amino)propylidene)-2-oxoindoline-5-carboxylic Acid

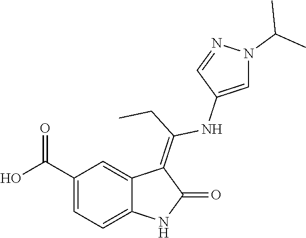 [SMILES: CC/C(Nc1cnn(C(C)C)c1)=C1/C(=O)Nc2ccc(C(=O)O)cc21]

A solution of ethyl (Z)-3-(1-((1-isopropyl-1H-pyrazol-4-yl)amino)propylidene)-2-oxoindoline-5-carboxylate (C1, 5.6 g, 15 mmol) in a mixture of MeOH:1,4-dioxane:H₂O (2:1:1, 80 mL) was treated with LiOH hydrate (1.03 g, 25 mmol). The reaction mixture was heated at 70° C. overnight. The reaction was cooled to rt and the volatiles removed under reduced pressure. The crude was purified via reverse phase column chromatography (0 to 100% MeCN/H₂O (0.1% HCOOH)) to obtain (Z)-3-(1-((1-isopropyl-1H-pyrazol-4-yl)amino)propylidene)-2-oxoindoline-5-carboxylic acid (1.16 g, 23%) as a brown solid. ¹H NMR (500 MHz, DMSO-d₆): δ 12.41 (s, 1H), 11.68 (s, 1H), 10.91 (s, 1H), 8.01 (s, 1H), 7.86 (s, 1H), 7.67 (dd, J=8.1, 1.5 Hz, 1H), 7.56 (s, 1H), 6.96 (d, J=8.1 Hz, 1H), 4.50 (m, 1H), 2.76 (q, J=7.6 Hz, 2H), 1.44 (d, J=6.8 Hz, 6H), 1.26 (t, J=7.6 Hz, 3H); MS (ESI) m/z: 341.2 (M+H⁺).

Using the General Method F above, the following Intermediates of Table D were prepared.

TABLE D

| Ex | | | Yield | ¹H NMR (400 or 500 MHz, | MS (m/z: |
|---|---|---|---|---|---|
| No | SM | Product | (%) | DMSO-d₆): δ | M + H⁺) |
| D2 | C2 | 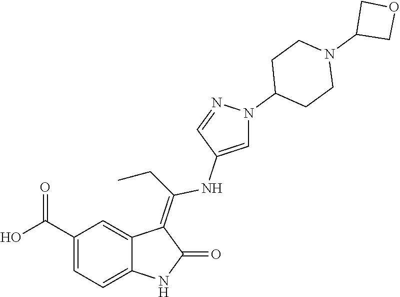 [SMILES: CC/C(Nc1cnn(C2CCN(C3COC3)CC2)c1)=C1/C(=O)Nc2ccc(C(=O)O)cc21] | 61 | No Data | 438.2 |
| D3 | C3 | 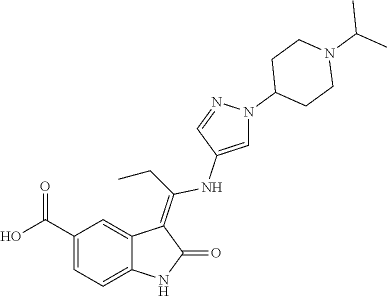 [SMILES: CC/C(Nc1cnn(C2CCN(C(C)C)CC2)c1)=C1/C(=O)Nc2ccc(C(=O)O)cc21] | 57 | 11.66 (s, 1H), 10.91 (s, 1H), 8.03 (s, 1H), 7.86 (d, J = 1.5 Hz, 1H), 7.66 (dd, J = 8.2, 1.5 Hz, 1H), 7.57 (s, 1H), 6.96 (d, J = 8.1 Hz, 1H), 4.12 (m, 2H), 2.91 (d, J = 11.0 Hz, 2H), 2.72-2.81 (m, 3H), 2.26-2.34 (m, 2H), 2.00-2.07 (m, 2H), 1.87-1.96 (m, 2H), 1.25 (t, J = 7.6 Hz, 3H), 1.00 (d, J = 6.5 Hz, 6H). | 424.2 |

General Method G: Pd-catalyzed fluorocarbonylation using N-formylsaccarin

### Example E1: (Z)-3-(1-((1-(1-isopropylpiperidin-4-yl)-1H-pyrazol-4-yl)amino)propylidene)-2-oxoindoline-5-carbonyl Fluoride

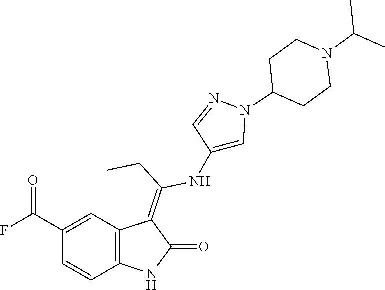 [SMILES: CC/C(Nc1cnn(C2CCN(C(C)C)CC2)c1)=C1/C(=O)Nc2ccc(C(=O)F)cc21]

A solution of (Z)-5-bromo-3-(1-((1-(1-isopropylpiperidin-4-yl)-1H-pyrazol-4-yl)amino)propylidene)indolin-2-one (C4, 0.50 g, 1.1 mmol) in DMF (7 mL) was treated with KF (0.16 g, 2.7 mmol). The reaction mixture was sparged with Ar for 5 min. Palladium(II) acetate (16 mg, 0.07 mmol) and xantphos (0.06 g, 0.11 mmol) were added and then the mixture was sparged with Ar for 5 min. N-formylsaccharin (0.74 g, 3.6 mmol) was added and the vial sealed immediately. The vial was then heated to 80° C. overnight. The reaction was cooled to rt and the cooled material was transferred to a 10 mL volumetric flask and diluted to the mark with fresh DMF. For the purposes of calculation, the conversion was assumed to be 100% and the final concentration of (Z)-3-(1-((1-(1-isopropylpiperidin-4-yl)-1H-pyrazol-4-yl)amino)propylidene)-2-oxoindoline-5-carbonyl fluoride (0.46 g, 100%) was assumed to be 0.1 M. No additional data was collected on the product.

Using the General Method G above, the following Intermediates of Table E were prepared

TABLE D

| Ex | | | Yield | ¹H NMR (400 or 500 MHz, | MS (m/z: |
|---|---|---|---|---|---|
| No | SM | Product | (%) | DMSO-d₆): δ | M + H⁺) |
| E2 | 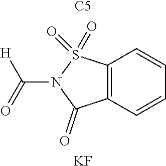 | 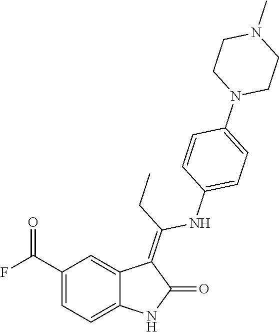 [SMILES: CC/C(Nc1ccc(N2CCN(C)CC2)cc1)=C1/C(=O)Nc2ccc(C(=O)F)cc21] | crude | No Data | 409.2 |

### General Method H: Substitution Reaction on Acyl Fluoride

### Example 1: (R,Z)-3-(1-((1-(1-isopropylpiperidin-4-yl)-1H-pyrazol-4-yl)amino)propylidene)-2-oxo-N-((tetrahydrofuran-2-yl)methyl)indoline-5-carboxamide

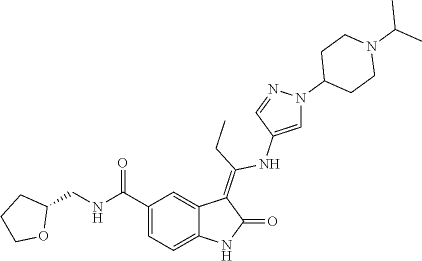 [SMILES: CC/C(Nc1cnn(C2CCN(C(C)C)CC2)c1)=C1/C(=O)Nc2ccc(C(=O)NC[C@H]3CCCO3)cc21]

A solution of (Z)-3-(1-((1-(1-isopropylpiperidin-4-yl)-1H-pyrazol-4-yl)amino)propylidene)-2-oxoindoline-5-carbonyl fluoride (E1, 0.14 g, 0.32 mmol) in DMF (3 mL) was treated with triethylamine (0.2 mL, 1.43 mmol) at rt. (R)-(tetrahydrofuran-2-yl)methanamine (0.07 g, 0.70 mmol) was added and the reaction was stirred at rt for 2 h. The volatiles were removed under reduced pressure and the residue was diluted with aqueous Na₂CO₃ (50%). The solution was extracted with DCM (4×) and the combined organics were dried over anhydrous Na₂SO₄, filtered, and concentrated under reduced pressure. The residue was purified via reverse phase column chromatography (5 to 90% H₂O/MeCN (0.1% HCOOH)). The major fractions were collected and treated with aqueous Na₂CO₃ (50%). The solution was extracted with DCM (4×) and the combined organic layers were dried over anhydrous Na₂SO₄, filtered, and concentrated under reduced pressure to obtain (R,Z)-3-(1-((1-(1-isopropylpiperidin-4-yl)-1H-pyrazol-4-yl)amino)propylidene)-2-oxo-N-((tetrahydrofuran-2-yl)methyl)indoline-5-carboxamide (0.10 g, 55%). ¹H NMR (500 MHz, DMSO-d₆): δ 11.70 (s, 1H), 10.77 (s, 1H), 8.36 (t, J=5.8 Hz, 1H), 8.01 (s, 1H), 7.75 (s, 1H), 7.55 (s, 1H), 7.54 (dd, J=8.1, 1.5 Hz, 1H), 6.90 (d, J=8.1 Hz, 1H), 4.09 (m, 1H), 3.97 (m, 1H), 3.76 (m, 1H), 3.61 (q, J=7.3 Hz, 1H), 3.25-3.35 (m, 2H), 2.87 (m, 2H), 2.79 (m, 2H), 2.72 (m, 1H), 2.25 (t, J=11.5 Hz, 2H), 2.01 (m, 2H), 1.83-1.92 (m, 3H), 1.75-1.82 (m, 2H), 1.58 (m, 1H), 1.22 (t, J=7.5 Hz, 3H), 0.98 (d, J=6.6 Hz, 6H); MS (ESI) m/z: 507.4 (M+H⁺).

Method I: Aminocarbonylation with Co₂(CO)₈

### Example 2: (Z)-3-(1-((1-(1-isopropylpiperidin-4-yl)-1H-pyrazol-4-yl)amino)propylidene)-N,N-dimethyl-2-oxoindoline-5-carboxamide

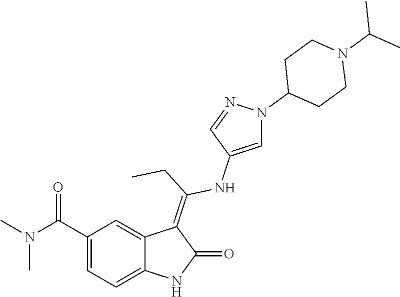 [SMILES: CC/C(Nc1cnn(C2CCN(C(C)C)CC2)c1)=C1/C(=O)Nc2ccc(C(=O)N(C)C)cc21]

A solution of (Z)-5-bromo-3-(1-((1-(1-isopropylpiperidin-4-yl)-1H-pyrazol-4-yl)amino)propylidene)indolin-2-one (C4, 200 mg, 0.44 mmol) in 1,4-dioxane (2 mL) was treated with dimethylamine in 2.0 M in THF (0.5 mL, 1.0 mmol). Palladium(II) acetate (6 mg, 0.026 mmol), xantphos (18 mg, 0.031 mmol), N,N-dimethylpyridin-4-amine (110 mg, 0.90 mmol), and Co₂(CO)₈ (37 mg, 0.11 mmol) were added. The vial was sealed and heated to 90° C. in the microwave for 2.5 h. The reaction was diluted with EtOAc (10 mL) and the blue solids filtered off and rinsed with EtOAc (5 mL). The filtrate was concentrated under reduced pressure. The crude was purified via reversed phase column chromatography (0 to 90% MeCN/H₂O (0.1% HCOOH)). The major peak was collected and treated with sat'd NaHCO₃ solution and stirred at rt for 30 min. The solution was extracted with 20% MeOH/DCM (3×) and the combined organic extracts were concentrated under reduced pressure. The residue was purified via Sfar KP Amino D column chromatography (0 to 20% MeOH/DCM) to obtain (Z)-3-(1-((1-(1-isopropylpiperidin-4-yl)-1H-pyrazol-4-yl)amino)propylidene)-N,N-dimethyl-2-oxoindoline-5-carboxamide (160 mg, 78%). ¹H NMR (500 MHz, DMSO-d₆): δ 11.68 (s, 1H), 10.71 (s, 1H), 8.01 (s, 1H), 7.54 (s, 1H), 7.27 (s, 1H), 7.06 (d, J=8.0 Hz, 1H), 6.90 (d, J=8.0 Hz, 1H), 4.09 (m, 1H), 2.96 (s, 6H), 2.82-2.92 (m, 2H), 2.71 (q, J=7.7 Hz, 3H), 2.25 (m, 2H), 2.01 (m, 2H), 1.89 (m, 2H), 1.21 (t, J=7.6 Hz, 3H), 0.98 (d, J=6.5 Hz, 6H); MS (ESI) m/z: 451.2 (M+H⁺).

Method J: Aminocarbonylation with Mo(CO)₆

### Example 3: (Z)—N-((5-cyclopropyltetrahydrofuran-2-yl)methyl)-3-(1-((1-(1-isopropylpiperidin-4-yl)-1H-pyrazol-4-yl)amino)propylidene)-2-oxoindoline-5-carboxamide

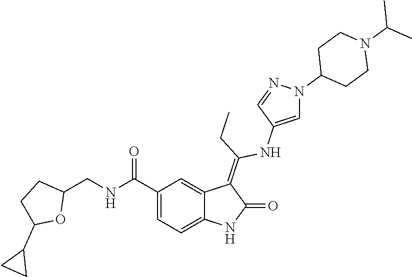 [SMILES: CC/C(Nc1cnn(C2CCN(C(C)C)CC2)c1)=C1/C(=O)Nc2ccc(C(=O)NCC3CCC(C4CC4)O3)cc21]

A solution of (Z)-5-bromo-3-(1-((1-(1-isopropylpiperidin-4-yl)-1H-pyrazol-4-yl)amino)propylidene)indolin-2-one (C4, 0.25 g, 0.55 mmol) in 1,4-dioxane (5 mL) was treated with Na₂CO₃ (0.18 g, 1.7 mmol) and (5-cyclopropyltetrahydrofuran-2-yl)methanamine (0.13 g, 0.93 mmol). The mixture was sparged with Ar for 2 min and then Mo(CO)₆ (0.072 g, 0.27 mmol) and Herrmann-Beller catalyst (0.051 g, 0.054 mmol) were added. The reaction mixture was sealed and heated to 150° C. for 6 h. The reaction was diluted with DCM (20 mL), filtered through a pad of celite. The filtrate was washed with sat'd NaHCO₃ and the organics removed. The aq phase extracted with DCM (2×) and the combined organics were dried over anhydrous MgSO₄, filtered, and concentrated under reduced pressure. The residue was purified via silica gel column chromatography (20% (1% NH₄OH/MeOH)/DCM and 0-40% DCM/MeOH). The fractions were combined and concentrated under reduced pressure and the residue was treated with MeCN. The solution was sonicated for 10 min. The solids were filtered and washed with MeCN to obtain (Z)—N-((5-cyclopropyltetrahydrofuran-2-yl)methyl)-3-(1-((1-(1-isopropylpiperidin-4-yl)-1H-pyrazol-4-yl)amino)propylidene)-2-oxoindoline-5-carboxamide (0.11 g, 35%) as a white solid. ¹H NMR (500 MHz, DMSO-d₆): δ 11.71 (d, J=1.9 Hz, 1H), 10.78 (s, 1H), 8.36 (m, 1H), 8.02 (s, 1H), 7.76 (m, 1H), 7.56 (m, 2H), 6.91 (d, J=8.1 Hz, 1H), 3.92-4.13 (m, 2H), 3.38 (q, J=7.0 Hz, 1H), 3.15-3.30 (m, 2H), 2.68-2.94 (m, 5H), 2.17-2.34 (br m, 2H), 1.83-2.07 (m, 6H), 1.54-1.71 (m, 2H), 1.23 (t, J=7.6 Hz, 3H), 0.99 (d, J=6.6 Hz, 6H), 0.85 (m, 1H), 034-0.47 (m, 2H), 0.12-0.27 (m, 2H); MS (ESI) m/z: 547.4 (M+H⁺).

### Method K: Substitution Reaction

### Example 4: (Z)-3-(1-((1-isopropyl-1H-pyrazol-4-yl)amino)propylidene)-2-oxo-N-(2,2,2-trifluoroethyl)indoline-5-carboxamide

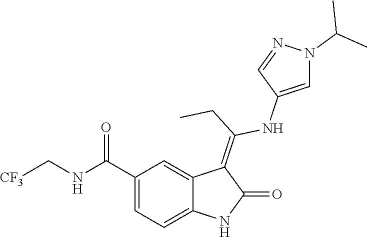 [SMILES: CC/C(Nc1cnn(C(C)C)c1)=C1/C(=O)Nc2ccc(C(=O)NCC(F)(F)F)cc21]

A solution of (Z)-3-(1-ethoxypropylidene)-2-oxo-N-(2,2,2-trifluoroethyl)indoline-5-carboxamide (B1, 0.25 g, 0.73 mmol) in EtOH (2 mL) was treated with 1-isopropyl-1H-pyrazol-4-amine (0.091 g, 0.73 mmol). The reaction was heated to 80° C. overnight. The reaction was cooled to rt and concentrated under reduced pressure. The crude was purified via silica gel column chromatography (0 to 100% MeOH/DCM) to obtain a light orange solid. The orange solid was triturated from MeCN and then the solids were filtered to obtain (Z)-3-(1-((1-methyl-1H-pyrazol-4-yl)amino)propylidene)-2-oxo-N-(2,2,2-trifluoroethyl)indoline-5-carboxamide (0.14 g, 46%) as a pale orange solid. ¹H NMR (500 MHz, DMSO-d₆): δ 11.75 (s, 1H), 10.87 (s, 1H), 8.90 (t, J=6.3 Hz, 1H), 8.02 (s, 1H), 7.81 (d, J=1.6 Hz, 1H), 7.62 (dd, J=8.2, 1.6 Hz, 1H), 7.57 (s, 1H), 6.96 (d, J=8.1 Hz, 1H), 4.51 (m, 1H), 4.09 (m, 2H), 2.83 (q, J=7.6 Hz, 2H), 1.45 (d, J=6.6 Hz, 6H), 1.26 (t, J=7.5 Hz, 3H); MS (ESI) m/z: 422.2 (M+H⁺).

General Method L: Amide formation

### Example 5: (Z)-3-(1-((1-isopropyl-1H-pyrazol-4-yl)amino)propylidene)-N-((3-methyloxetan-3-yl)methyl)-2-oxoindoline-5-carboxamide

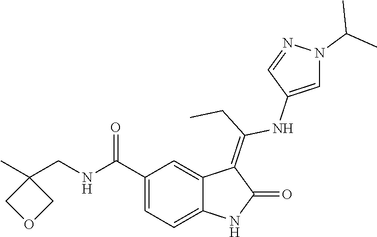 [SMILES: CC/C(Nc1cnn(C(C)C)c1)=C1/C(=O)Nc2ccc(C(=O)NCC3(C)COC3)cc21]

A solution of (Z)-3-(1-((1-isopropyl-1H-pyrazol-4-yl)amino)propylidene)-2-oxoindoline-5-carboxylic acid (D1, 0.15 g, 0.44 mmol) in DCM (5 mL) was treated with EDC (0.10 g, 0.53 mmol) and HOBt (10 mol %). The reaction was allowed to stir for 10 min and then DIEA (0.096 mL, 0.55 mmol) and (3-Methyloxetan-3-yl)methanamine (0.053 g, 0.53 mmol) was added and the reaction stirred at rt overnight. The reaction was quenched with sat'd NaHCO₃ (30 mL) and then the solution was extracted with DCM (3×). The combined organics were dried over anhydrous MgSO₄, filtered, and concentrated under reduced pressure. The crude was purified via silica gel column chromatography (0 to 20% DCM/MeOH) to obtain (Z)-3-(1-((1-isopropyl-1H-pyrazol-4-yl)amino)propylidene)-N-((3-methyloxetan-3-yl)methyl)-2-oxoindoline-5-carboxamide (0.02 g, 12%) as a white solid. ¹H NMR (500 MHz, DMSO-d₆): δ 11.71 (s, 1H), 10.79 (s, 1H), 8.47 (t, J=6.1 Hz, 1H), 8.00 (s, 1H), 7.77 (s, 1H), 7.56 (d, J=7.5 Hz, 2H), 6.93 (d, J=8.1 Hz, 1H), 4.47-4.53 (m, 3H), 4.19 (d, J=5.7 Hz, 2H), 3.44 (d, J=6.0 Hz, 2H), 2.81 (q, J=7.6 Hz, 2H), 1.44 (d, J=6.7 Hz, 6H), 1.23-1.27 (m, 6H); MS (ESI) m/z: 424.2 (M+H⁺).

### General Method M: Suzuki Reaction and Oxidation

### Example 6a: (Z)-3-(1-((1-isopropyl-1H-pyrazol-4-yl)amino)propylidene)-2-oxoindoline-5-carbaldehyde

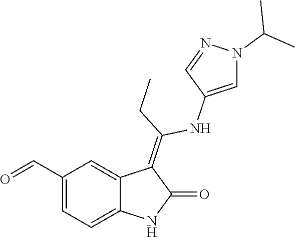 [SMILES: CC/C(Nc1cnn(C(C)C)c1)=C1/C(=O)Nc2ccc(C=O)cc21]

A stirred solution of potassium(I) trifluoro(vinyl)borate (4.3 g, 32.0 mmol) and (Z)-5-bromo-3-(1-((1-isopropyl-1H-pyrazol-4-yl)amino)propylidene)indolin-2-one (C7, 4.0 g, 10.6 mmol) in 1,4-dioxane:water (4:1, 45 mL), K₂CO₃ (4.4 g, 32.0 mmol) was added at rt and degassed the reaction mixture with nitrogen balloon for 20 min. After that PdCl₂(dppf).DCM (0.87 g, 1.0 mmol) was added and the reaction mixture was stirred at 100° C. for 20 h. The reaction mixture was filtered through a pad of celite and washed with EtOAc. The filtrate was concentrated under reduced pressure and the crude material was purified via silica gel column chromatography (40% EtOAc/hexanes) to obtain (Z)-3-(1-((1-isopropyl-1H-pyrazol-4-yl)amino)propylidene)-5-vinylindolin-2-one (5.0 g, 73%) as an off white solid. ¹H NMR (400 MHz, DMSO-d₆): δ 11.67 (s, 1H), 10.59 (s, 1H), 7.98 (s, 1H), 7.53 (s, 1H), 7.33 (s, 1H), 7.12 (d, J=8.0 Hz, 1H), 6.85 (d, J=8.0 Hz, 1H), 5.62 (d, J=17.6 Hz, 1H), 5.06 (d, J=10.8 Hz, 1H), 4.49 (t, J=6.4 Hz, 1H), 2.75 (q, J=7.6 Hz, 2H), 1.42 (d, J=6.4 Hz, 6H), 1.24 (br s, 3H); MS (ESI) m/z: 323.1 (M+H⁺).

A solution of (Z)-3-(1-((1-isopropyl-1H-pyrazol-4-yl)amino)propylidene)-5-vinylindolin-2-one (3.0 g, 9.3 mmol) in THF:H₂O (1:1, 60 mL) was treated with OsO₄ (1.2 mL, 0.19 mmol) at 0° C., followed by addition of NaIO₄ (3.0 g, 14.0 mmol). The reaction mixture was stirred at 0° C. for 30 min. The reaction mixture was treated with water (70 mL) and extracted with EtOAc (2×). The combined organic extracts were washed with brine, dried over anhydrous Na₂SO₄, filtered, and concentrated under reduced pressure. The crude material was purified via silica gel column chromatography (70% EtOAc/hexanes) to obtain (Z)-3-(1-((1-isopropyl-1H-pyrazol-4-yl)amino)propylidene)-2-oxoindoline-5-carbaldehyde (1.0 g, 33%) as a pale-yellow solid. ¹H NMR (400 MHz, DMSO-d₆): δ 11.70 (s, 1H), 11.08 (s, 1H), 9.88 (s, 1H), 8.02 (s, 1H), 7.78 (s, 1H), 7.58 (br m, 2H), 7.06 (d, J=8.0 Hz, 1H), 4.50 (m, 1H), 2.79 (q, J=7.6 Hz, 2H), 1.43 (d, J=6.8 Hz, 6H), 1.05 (t, J=6.8 Hz, 3H); MS (ESI) m/z: 325.0 (M+H⁺).

### General Method N: Grignard Reaction and Reduction

### Example 6: (Z)-5-(cyclopropanecarbonyl)-3-(1-((1-isopropyl-1H-pyrazol-4-yl)amino)propylidene)indolin-2-one

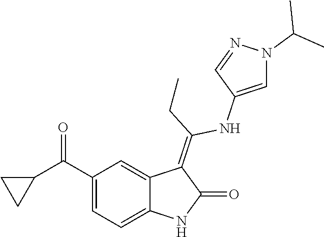 [SMILES: CC/C(Nc1cnn(C(C)C)c1)=C1/C(=O)Nc2ccc(C(=O)C3CC3)cc21]

A solution of (Z)-3-(1-((1-isopropyl-1H-pyrazol-4-yl)amino)propylidene)-2-oxoindoline-5-carbaldehyde (6a, 1.0 g, 3.1 mmol) in THF was cooled to 0° C. and 0.5 M cyclopropylmagnesium bromide (49 mL, 24 mmol in THF) was added. The reaction mixture was warmed to rt and stirred for 4 h. The reaction mixture was cooled to 0° C. and quenched with sat'd NH₄Cl solution. The solution was extracted with EtOAc (2×). The combined organic extracts were washed with water (2×), dried over anhydrous Na₂SO₄, filtered, and concentrated under reduced pressure. The crude material was purified via silica gel column chromatography (EtOAc/hexanes) to obtain (Z)-5-(cyclopropyl(hydroxy)methyl)-3-(1-((1-isopropyl-1H-pyrazol-4-yl)amino)propylidene)indolin-2-one (0.5 g, 45%) as an off white solid. ¹H NMR (400 MHz, DMSO-d₆): δ 11.64 (s, 1H), 10.45 (s, 1H), 7.97 (s, 1H), 7.52 (s, 1H), 7.30 (s, 1H), 6.98 (d, J=8.0 Hz, 1H), 6.80 (d, J=7.6 Hz, 1H), 5.00 (d, J=4.0 Hz, 1H), 4.49 (m, 1H), 3.94 (m, 1H), 2.75 (q, J=7.2 Hz, 2H), 1.42 (d, J=8.0 Hz, 6H), 1.23 (t, J=7.6 Hz, 3H), 0.27-0.45 (m, 4H); MS (ESI) m/z: 367.2 (M+H⁺).

A solution of (Z)-5-(cyclopropyl(hydroxy)methyl)-3-(1-((1-isopropyl-1H-pyrazol-4-yl)amino)propylidene)indolin-2-one (0.50 g, 1.36 mmol) in DCM was cooled to 0° C. and Dess-Martin reagent (1.15 g, 1.36 mmol) was added slowly. The reaction mixture was stirred at 0° C. for 30 min. The reaction mixture was filtered through a pad of celite and washed with DCM. The filtrate was washed with water (50 mL) and dried over anhydrous Na₂SO₄, filtered, and concentrated under reduced pressure. The crude material was purified via silica gel column chromatography (EtOAc/hexanes) to obtain (Z)-5-(cyclopropanecarbonyl)-3-(1-((1-isopropyl-1H-pyrazol-4-yl)amino)propylidene)indolin-2-one (0.040 g, 8%) as an off white solid. ¹H NMR (400 MHz, DMSO-d₆): δ 11.70 (s, 1H), 10.96 (s, 1H), 8.00 (s, 1H), 7.86 (d, J=8.0 Hz, 1H), 7.84 (d, J=1.2 Hz, 1H), 7.56 (s, 1H), 6.99 (d, J=8.0 Hz, 1H), 4.50 (m, 1H), 2.76-2.89 (m, 3H), 1.42 (d, J=6.8 Hz, 6H), 1.26 (t, J=7.6 Hz, 3H), 0.98 (m, 4H); MS (ESI) m/z: 365.2 (M+H⁺).

Using the General Methods H, I, J, K, L, M, and N above, the following Compounds of Table E were prepared.

TABLE E

| Ex | | | | Yield | ¹H NMR (400 or 500 | MS (m/z: |
|---|---|---|---|---|---|---|
| No | SM | Product | Method | (%) | MHz, DMSO-d₆): δ | M + H⁺) |
| 7 | 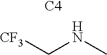 | 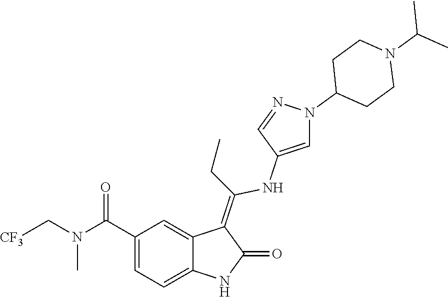 [SMILES: CC/C(Nc1cnn(C2CCN(C(C)C)CC2)c1)=C1/C(=O)Nc2ccc(C(=O)N(C)CC(F)(F)F)cc21] | I | 19 | 11.71 (s, 1H), 10.78 (s, 1H), 8.01 (s, 1H), 7.55 (s, 1H), 7.27 (s, 1H), 7.07 (d, J = 8.0 Hz, 1H), 6.94 (d, J = 8.0 Hz, 1H), 4.31 (m, 2H), 4.09 (m, 1H), 3.08 (s, 3H), 2.87 (m, 2H), 2.71 (q, J = 7.2 Hz, 3H), 2.24 (m, 2H), 2.01 (m, 2H), 1.89 (m, 2H), 1.20 (t, J = 7.6 Hz, 3H), 0.97 (d, J = 6.6 Hz, 6H). | 519.2 |
| 8 | 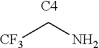 | 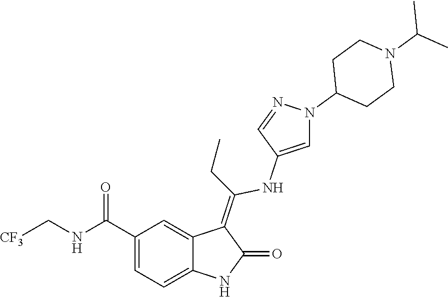 [SMILES: CC/C(Nc1cnn(C2CCN(C(C)C)CC2)c1)=C1/C(=O)Nc2ccc(C(=O)NCC(F)(F)F)cc21] | I | 47 | 11.71 (s, 1H), 10.85 (s, 1H), 8.89 (t, J = 6.3 Hz, 1H), 8.02 (s, 1H), 7.79 (s, 1H), 7.60 (dd, J = 8.2, 1.6 Hz, 1H), 7.56 (s, 1H), 6.94 (d, J = 8.2 Hz, 1H), 4.03-4.12 (m, 2H), 3.84 (m, 1H), 2.87 (m, 1H), 2.75 (m, 2H), 2.58 (m, 2H), 2.25 (m, 2H), 2.01 (m, 2H), 1.89 (m, 2H), 1.22 (t, J = 7.6 Hz, 3H), 0.98 (d, J = 6.6 Hz, 6H). | 505.2 |
| 9 | 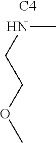 | 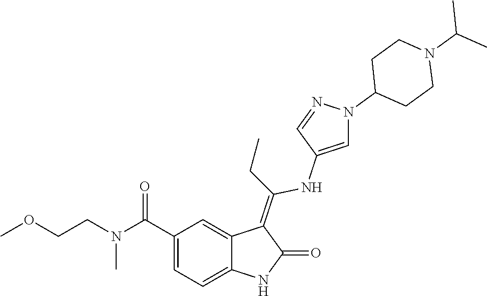 [SMILES: CC/C(Nc1cnn(C2CCN(C(C)C)CC2)c1)=C1/C(=O)Nc2ccc(C(=O)N(C)CCOC)cc21] | I | 43 | 11.69 (s, 1H), 10.70 (s, 1H), 8.01 (s, 1H), 7.54 (s, 1H), 7.27 (s, 1H), 7.03 (d, J = 8.0 Hz, 1H), 6.90 (d, J = 7.9 Hz, 1H), 4.09 (m, 1H), 3.49 (br s, 4H), 3.22 (br s, 3H), 2.97 (s, 3H), 2.87 (m, 2H), 2.72 (q, J = 7.2 Hz, 3H), 2.24 (m, 2H), 2.01 (m, 2H), 1.88 (m, 2H), 1.21 (t, J = 7.6 Hz, 3H), 0.97 (d, J = 6.6 Hz, 6H). | 495.3 |
| 10 | 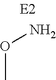 | 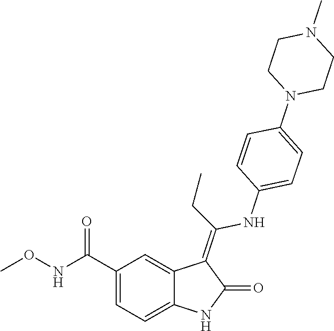 [SMILES: CC/C(Nc1ccc(N2CCN(C)CC2)cc1)=C1/C(=O)Nc2ccc(C(=O)NOC)cc21] | H | 24 | 11.98 (s, 1H), 11.55 (s, 1H), 10.81 (s, 1H), 7.65 (s, 1H), 7.44 (dd, J = 8.1, 1.5 Hz, 1H), 7.16 (d, J = 8.6 Hz, 2H), 7.00 (d, J = 8.6 Hz, 2H), 6.92 (d, J = 8.1 Hz, 1H), 3.69 (s, 3H), 3.16 (m, 4H), 2.76 (q, J = 7.2 Hz, 2H), 2.45 (m, 4H), 2.22 (s, 3H), 1.21 (t, J = 7.4 Hz, 3H). | 436.2 |
| 11 | 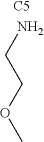 | 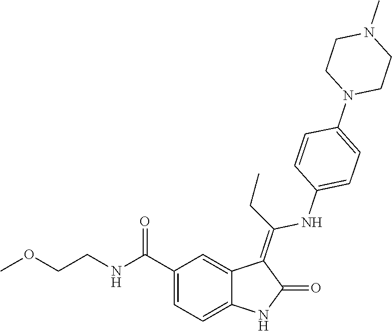 [SMILES: CC/C(Nc1ccc(N2CCN(C)CC2)cc1)=C1/C(=O)Nc2ccc(C(=O)NCCOC)cc21] | I | 33 | 11.97 (s, 1H), 10.76 (s, 1H), 8.34 (t, J = 5.4 Hz, 1H), 7.75 (s, 1H), 7.54 (dd, J = 8.2, 1.5 Hz, 1H), 7.16 (d, J = 8.6 Hz, 2H), 7.00 (d, J = 8.6 Hz, 2H), 6.90 (d, J = 8.1 Hz, 1H), 3.38- 3.45 (m, 4H), 3.26 (s, 3H), 3.17 (m, 4H), 2.77 (q, J = 7.6 Hz, 2H), 2.45 (m, 4H), 2.22 (s, 3H), 1.21 (t, J = 7.6 Hz, 3H). | 464.2 |
| 12 | 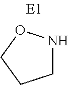 | 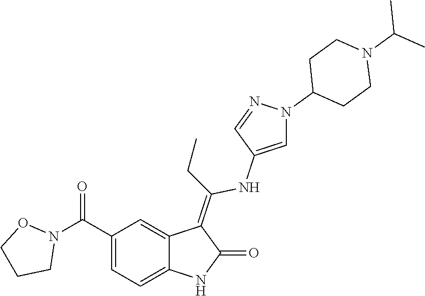 [SMILES: CC/C(Nc1cnn(C2CCN(C(C)C)CC2)c1)=C1/C(=O)Nc2ccc(C(=O)N3CCCO3)cc21] | I | 33 | 11.65 (s, 1H), 10.81 (s, 1H), 8.01 (s, 1H), 7.66 (s, 1H), 7.55 (s, 1H), 7.43 (dd, J = 8.1, 1.5 Hz, 1H), 6.92 (d, J = 8.1 Hz, 1H), 4.09 (m, 1H), 3.87 (t, J = 6.9 Hz, 2H), 3.75 (m, 2H), 2.87 (m, 2H), 2.73 (m, 3H), 2.23 (m, 4H), 2.01 (m, 2H), 1.90 (m, 2H), 1.23 (t, J = 7.6 Hz, 3H), 0.98 (d, J = 6.5 Hz, 6H). | 479.2 |
| 13 | C4 —NH₂ | 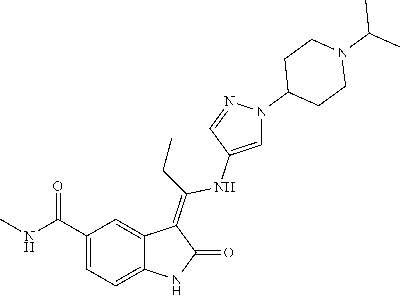 [SMILES: CC/C(Nc1cnn(C2CCN(C(C)C)CC2)c1)=C1/C(=O)Nc2ccc(C(=O)NC)cc21] | J | 27 | 11.69 (s, 1H), 10.75 (s, 1H), 8.24 (d, J = 5.1 Hz, 1H), 8.01 (s, 1H), 7.80 (m, 1H), 7.73 (s, 1H), 7.42-7.55 (m, 3H), 6.89 (d, J = 8.1 Hz, 1H), 4.09 (m, 1H), 2.86 (m, 2H), 2.69- 2.80 (m, 2H), 2.71 (s, 3H), 2.24 (dd, J = 12.5, 10.5 Hz, 2H), 2.01 (m, 2H), 1.89 (m, 2H), 1.22 (t, J = 7.5 Hz, 3H), 0.97 (d, J = 6.6 Hz, 6H). | 437.2 |
| 14 | 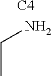 | 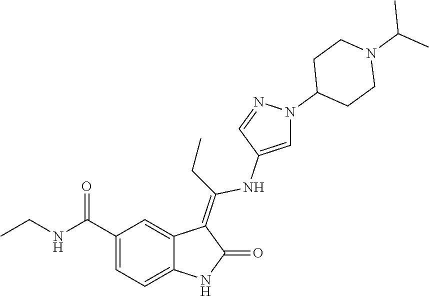 [SMILES: CCNC(=O)c1ccc2c(c1)/C(=C(\CC)Nc1cnn(C3CCN(C(C)C)CC3)c1)C(=O)N2] | J | 45 | 11.69 (s, 1H), 10.76 (s, 1H), 8.29 (m, 1H), 8.01 (s, 1H), 7.74 (s, 1H), 7.55 (s, 1H), 7.53 (d, J = 8.3 Hz, 1H), 6.90 (d, J = 8.1 Hz, 1H), 4.09 (m, 1H), 3.16 (m, 2H), 2.87 (m, 2H), 2.78 (m, 2H), 2.72 (m, 1H), 2.25 (m, 2H), 2.01 (m, 2H), 1.89 (m, 2H), 1.23 (t, J = 7.5 Hz, 3H), 1.11 (t, J = 7.2 Hz, 3H), 0.98 (d, J = 6.6 Hz, 6H). | 451.4 |
| 15 | 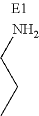 | 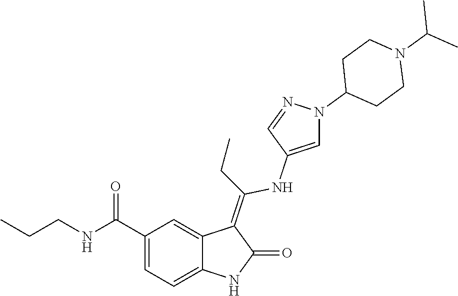 [SMILES: CCCNC(=O)c1ccc2c(c1)/C(=C(\CC)Nc1cnn(C3CCN(C(C)C)CC3)c1)C(=O)N2] | I | 36 | 11.69 (s, 1H), 10.76 (s, 1H), 8.28 (t, J = 5.6 Hz, 1H), 8.01 (s, 1H), 7.74 (s, 1H), 7.55 (s, 1H), 7.53 (dd, J = 8.2, 1.5 Hz, 1H), 6.90 (d, J = 8.1 Hz, 1H), 4.09 (m, 1H), 3.20 (q, J = 6.5 Hz, 2H), 2.83-2.91 (m, 2H), 2.79 (q, J = 7.5 Hz, 2H), 2.66-2.76 (m, 1H), 2.25 (m, 2H), 2.01 (m, 2H), 1.83-1.95 (m, 2H), 1.48-1.55 (m, 2H), 1.23 (t, J = 7.6 Hz, 3H), 0.98 (d, J = 6.5 Hz, 6H), 0.88 (t, J = 7.4 Hz, 3H). | 465.4 |
| 16 | 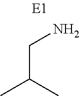 | 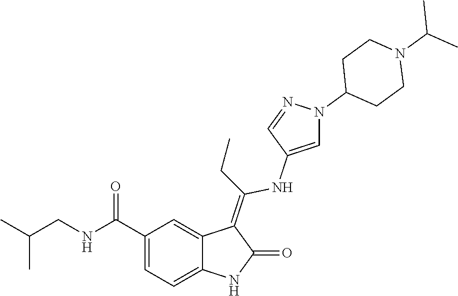 [SMILES: CC/C(Nc1cnn(C2CCN(C(C)C)CC2)c1)=C1/C(=O)Nc2ccc(C(=O)NCC(C)C)cc21] | I | 58 | 11.69 (s, 1H), 10.76 (s, 1H), 8.28 (t, J = 5.8 Hz, 1H), 8.01 (s, 1H), 7.74 (s, 1H), 7.55 (s, 1H), 7.54 (dd, J = 8.1, 1.5 Hz, 1H), 6.90 (d, J = 8.1 Hz, 1H), 4.09 (m, 1H), 3.06 (t, J = 6.4 Hz, 2H), 2.87 (d, J = 11.2 Hz, 2H), 2.80 (q, J = 7.7 Hz, 2H), 2.73 (m, 1H), 2.25 (t, J = 11.5 Hz, 2H), 2.01 (d, J = 11.9 Hz, 2H), 1.78- 1.93 (m, 3H), 1.23 (t, J = 7.5 Hz, 3H), 0.98 (d, J = 6.6 Hz, 6H), 0.88 (d, J = 6.7 Hz, 6H). | 479.4 |
| 17 | 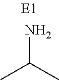 | 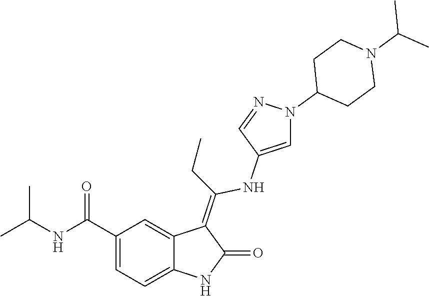 [SMILES: CC/C(Nc1cnn(C2CCN(C(C)C)CC2)c1)=C1/C(=O)Nc2ccc(C(=O)NC(C)C)cc21] | I | 35 | 11.67 (s, 1H), 10.76 (s, 1H), 8.01 (s, 1H), 8.00 (m, 1H), 7.74 (s, 1H), 7.55 (s, 1H), 7.53 (m, 1H), 6.88 (d, J = 8.1 Hz, 1H), 4.09 (m, 2H), 2.87 (m, 2H), 2.69- 2.80 (m, 3H), 2.25 (m, 2H), 2.01 (m, 2H), 1.89 (m, 2H), 1.23 (t, J = 7.6 Hz, 3H), 1.15 (d, J = 6.6 Hz, 6H), 0.98 (d, J = 6.6 Hz, 6H). | 465.4 |
| 18 | 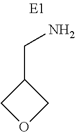 | 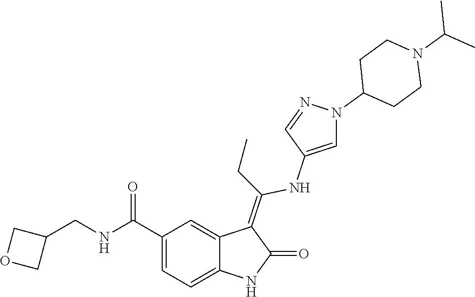 [SMILES: CC/C(Nc1cnn(C2CCN(C(C)C)CC2)c1)=C1/C(=O)Nc2ccc(C(=O)NCC3COC3)cc21] | I | 40 | 11.69 (s, 1H), 10.78 (s, 1H), 8.43 (t, J = 5.7 Hz, 1H), 8.01 (s, 1H), 7.74 (s, 1H), 7.55 (s, 1H), 7.53 (dd, J = 8.2, 1.5 Hz, 1H), 6.90 (d, J = 8.1 Hz, 1H), 4.61 (m, 2H), 4.34 (t, J = 6.0 Hz, 2H), 4.09 (m, 1H), 3.51 (t, J = 6.3 Hz, 2H), 3.15 (m, 1H), 2.87 (m, 2H), 2.79 (q, J = 7.6 Hz, 2H), 2.72 (m, 1H), 2.25 (m, 2H), 2.01 (m, 2H), 1.89 (m, 2H), 1.23 (t, J = 7.6 Hz, 3H), 0.98 (d, J = 6.6 Hz, 6H). | 493.3 |
| 19 | 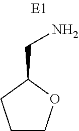 | 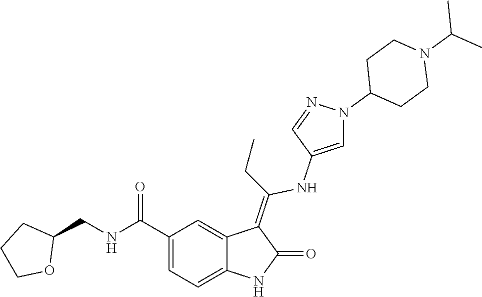 [SMILES: CC/C(Nc1cnn(C2CCN(C(C)C)CC2)c1)=C1/C(=O)Nc2ccc(C(=O)NC[C@@H]3CCCO3)cc21] | I | 38 | 11.70 (s, 1H), 10.77 (s, 1H), 8.36 (t, J = 5.8 Hz, 1H), 8.01 (s, 1H), 7.75 (s, 1H), 7.55 (s, 1H), 7.54 (m, 1H), 6.90 (d, J = 8.1 Hz, 1H), 4.09 (m, 1H), 3.97 (m, 1H), 3.77 (m, 1H), 3.61 (m, 1H), 3.25-3.35 (m, 2H), 2.87 (m, 2H), 2.79 (m, 2H), 2.72 (m, 1H), 2.25 (t, J = 11.5 Hz, 2H), 2.01 (m, 2H), 1.74- 1.93 (m, 5H), 1.58 (m, 1H), 1.23 (t, J = 7.5 Hz, 3H), 0.98 (d, J = 6.6 Hz, 6H). | 507.4 |
| 20 | 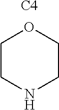 | 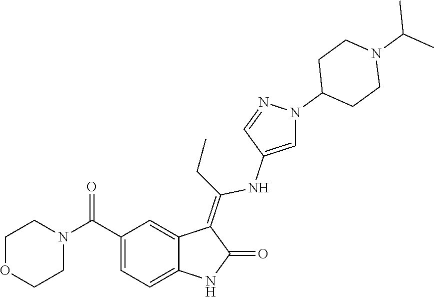 [SMILES: CC/C(Nc1cnn(C2CCN(C(C)C)CC2)c1)=C1/C(=O)Nc2ccc(C(=O)N3CCOCC3)cc21] | J | 55 | 11.69 (s, 1H), 10.73 (s, 1H), 8.01 (s, 1H), 7.54 (s, 1H), 7.27 (s, 1H), 7.06 (m, 1H), 6.92 (d, J = 7.9 Hz, 1H), 4.09 (m, 1H), 3.59 (br s, 4H), 3.51 (br s, 4H), 2.87 (m, 2H), 2.69-2.75 (m, 3H), 2.25 (t, J = 11.5 Hz, 2H), 2.01 (m, 2H), 1.89 (m, 2H), 1.21 (t, J = 7.6 Hz, 3H), 0.98 (d, J = 6.6 Hz, 6H). | 493.2 |
| 21 | 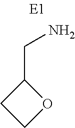 | 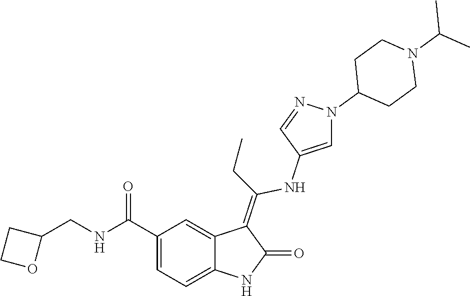 [SMILES: CC/C(Nc1cnn(C2CCN(C(C)C)CC2)c1)=C1/C(=O)Nc2ccc(C(=O)NCC3CCO3)cc21] | I | 37 | 11.70 (s, 1H), 10.78 (s, 1H), 8.49 (t, J = 5.8 Hz, 1H), 8.01 (s, 1H), 7.78 (s, 1H), 7.56-7.58 (m, 2H), 6.91 (d, J = 8.1 Hz, 1H), 4.82 (m, 1H), 4.49 (m, 1H), 4.41 (m, 1H), 4.09 (m, 1H), 3.54 (m, 1H), 3.45 (m, 1H), 2.87 (m, 2H), 2.81 (q, J = 7.8 Hz, 2H), 2.72 (m, 1H), 2.59 (m, 1H), 2.40 (m, 1H), 2.25 (m, 2H), 2.01 (m, 2H), 1.89 (m, 2H), 1.23 (t, J = 7.6 Hz, 3H), 0.92 (d, J = 6.6 Hz, 6H). | 493.3 |
| 22 | 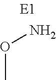 | 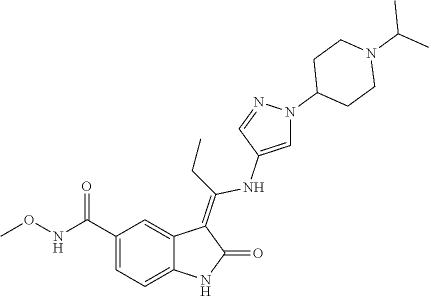 [SMILES: CC/C(Nc1cnn(C2CCN(C(C)C)CC2)c1)=C1/C(=O)Nc2ccc(C(=O)NOC)cc21] | I | 33 | 11.71 (s, 1H), 11.57 (s, 1H), 10.81 (s, 1H), 8.02 (s, 1H), 7.66 (s, 1H), 7.56 (s, 1H), 7.44 (dd, J = 8.1, 1.5 Hz, 1H), 6.92 (d, J = 8.1 Hz, 1H), 4.10 (m, 1H), 3.69 (s, 3H), 2.87 (m, 2H), 2.71-2.81 (m, 3H), 2.25 (m, 2H), 2.01 (m, 2H), 1.90 (m, 2H), 1.23 (t, J = 7.5 Hz, 3H), 0.98 (d, J = 6.6 Hz, 6H). | 453.2 |
| 23 | 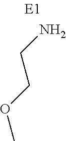 | 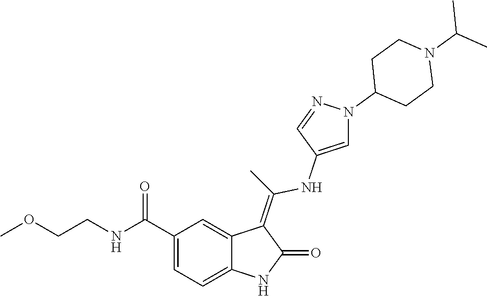 [SMILES: COCCNC(=O)c1ccc2c(c1)/C(=C(\C)Nc1cnn(C3CCN(C(C)C)CC3)c1)C(=O)N2] | I | 17 | 11.82 (s, 1H), 10.74 (s, 1H), 8.38 (t, J = 5.4 Hz, 1H), 8.00 (s, 1H), 7.84 (s, 1H), 7.55 (s, 1H), 7.54 (dd, J = 8.1, 1.6 Hz, 1H), 6.90 (d, J = 8.1 Hz, 1H), 4.05-4.10 (m, 1H), 3.38-3.45 (m, 4H), 3.26 (s, 3H), 2.87 (d, J = 11.0 Hz, 2H), 2.73 (t, J = 7.2 Hz, 1H), 2.51 (s, 3H), 2.25 (t, J = 11.5 Hz, 2H), 2.01 (d, J = 11.9 Hz, 2H), 1.85-1.92 (m, 2H), 0.98 (d, J = 6.6 Hz, 6H). | 467.4 |
| 24 | 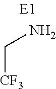 | 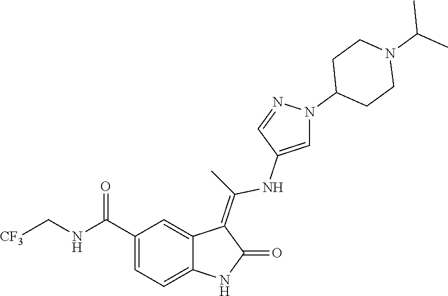 [SMILES: C/C(Nc1cnn(C2CCN(C(C)C)CC2)c1)=C1/C(=O)Nc2ccc(C(=O)NCC(F)(F)F)cc21] | I | 11 | 11.84 (s, 1H), 10.82 (s, 1H), 8.91 (t, J = 6.3 Hz, 1H), 8.00 (s, 1H), 7.88 (s, 1H), 7.59 (dd, J = 8.2, 1.6 Hz, 1H), 7.56 (s, 1H), 6.94 (d, J = 8.1 Hz, 1H), 4.08 (m, 3H), 2.88 (m, 2H), 2.72 (m, 1H), 2.53 (s, 3H), 2.25 (m, 2H), 2.01 (m, 2H), 1.89 (m, 2H), 0.98 (d, J = 6.6 Hz, 6H). | 491.2 |
| 25 | 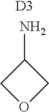 | 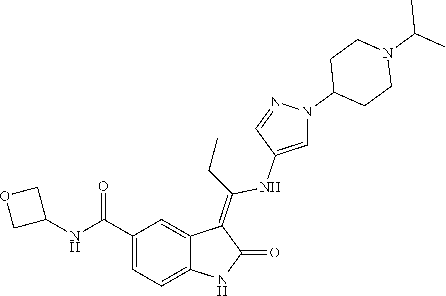 [SMILES: CC/C(Nc1cnn(C2CCN(C(C)C)CC2)c1)=C1/C(=O)Nc2ccc(C(=O)NC3COC3)cc21] | L | 24 | 11.69 (s, 1H), 10.84 (s, 1H), 8.92 (d, J = 6.4 Hz, 1H), 8.03 (s, 1H), 7.76 (s, 1H), 7.60 (d, J = 8.2 Hz, 1H), 7.56 (s, 1H), 6.92 (d, J = 8.1 Hz, 1H), 4.99 (m, 1H), 4.75 (t, J = 6.9 Hz, 2H), 4.59 (t, J = 6.4 Hz, 2H), 4.09 (m, 1H), 2.87 (m, 2H), 2.79 (q, J = 7.5 Hz, 2H), 2.73 (m, 1H), 2.24 (m, 2H), 2.00 (m, 2H), 1.89 (m, 2H), 1.23 (t, J = 7.6 Hz, 3H), 0.97 (d, J = 6.5 Hz, 6H). | 479.3 |
| 26 | 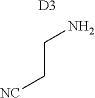 | 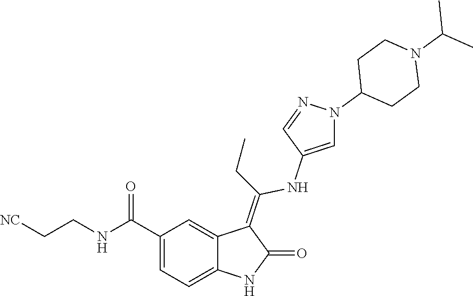 [SMILES: CC/C(Nc1cnn(C2CCN(C(C)C)CC2)c1)=C1/C(=O)Nc2ccc(C(=O)NCCC#N)cc21] | L | 26 | 11.70 (s, 1H), 10.84 (s, 1H), 8.70 (br s, 1H), 8.03 (s, 1H), 7.76 (d, J = 1.6 Hz, 1H), 7.56 (s, 1H), 7.55 (dd, J = 8.2, 1.6 Hz, 1H), 6.93 (d, J = 8.1 Hz, 1H), 4.09 (m, 1H), 3.47 (q, J = 6.3 Hz, 2H), 2.87 (m, 2H), 2.68-2.83 (m, 5H), 2.24 (m, 2H), 2.01 (m, 2H), 1.90 (m, 2H), 1.23 (t, J = 7.6 Hz, 3H), 0.98 (d, J = 6.5 Hz, 6H). | 476.2 |
| 27 | 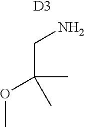 | 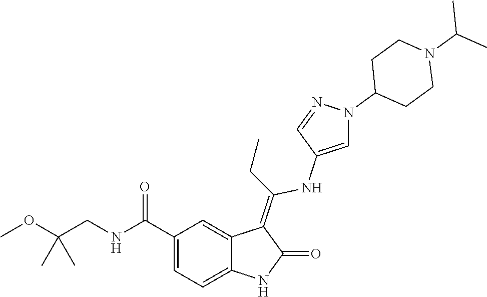 [SMILES: CC/C(Nc1cnn(C2CCN(C(C)C)CC2)c1)=C1/C(=O)Nc2ccc(C(=O)NCC(C)(C)OC)cc21] | L | 69 | 11.68 (s, 1H), 10.80 (s, 1H), 8.08 (br s, 1H), 8.02 (s, 1H), 7.74 (s, 1H), 7.56 (s, 1H), 7.54 (dd, J = 8.2, 1.5 Hz, 1H), 6.90 (d, J = 8.1 Hz, 1H), 4.09 (m, 1H), 3.30 (d, J = 6.2 Hz, 2H), 3.14 (s, 3H), 2.86 (m, 2H), 2.79 (q, J = 7.6 Hz, 2H), 2.73 (m, 1H), 2.24 (m, 2H), 2.00 (m, 2H), 1.89 (m, 2H), 1.23 (t, J = 7.6 Hz, 3H), 1.09 (s, 6H), 0.97 (d, J = 6.5 Hz, 6H). | 509.4 |
| 28 | 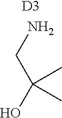 | 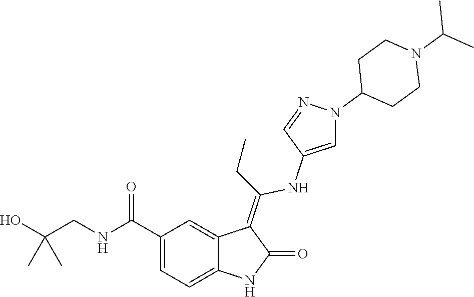 [SMILES: CC/C(Nc1cnn(C2CCN(C(C)C)CC2)c1)=C1/C(=O)Nc2ccc(C(=O)NCC(C)(C)O)cc21] | L | 20 | 11.69 (s, 1H), 10.81 (s, 1H), 8.12 (br s, 1H), 8.03 (s, 1H), 7.75 (d, J = 1.6 Hz, 1H), 7.56 (s, 1H), 7.55 (d, J = 8.1, 1.6 Hz, 1H), 6.91 (d, J = 8.1 Hz, 1H), 4.61 (s,1H), 4.09 (m, 1H), 3.23 (m, 2H), 2.87 (m, 2H), 2.79 (q, J = 7.6 Hz, 2H), 2.73 (m, 1H), 2.24 (m, 2H), 2.00 (m, 2H), 1.89 (m, 2H), 1.23 (t, J = 7.6 Hz, 3H), 1.09 (s, 6H), 0.97 (d, J = 6.5 Hz, 6H). | 495.4 |
| 29 | 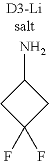 | 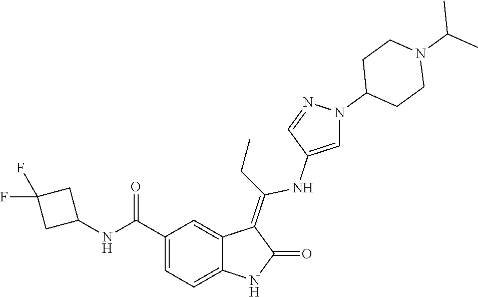 [SMILES: CC/C(Nc1cnn(C2CCN(C(C)C)CC2)c1)=C1/C(=O)Nc2ccc(C(=O)NC3CC(F)(F)C3)cc21] | L (TBTU) | 29 | 11.68 (s, 1H), 10.84 (s, 1H), 8.63 (d, J = 6.6 Hz, 1H), 8.03 (s, 1H), 7.74 (s, 1H), 7.52-7.58 (m, 2H), 6.92 (d, J = 8.1 Hz, 1H), 4.25 (p, J = 7.3 Hz, 1H), 4.09 (bs, 1H), 3.33 (s, 4H), 2.84- 2.98 (m, 2H), 2.66- 2.82 (m, 3H), 2.24 (t, J = 11.5 Hz, 2H), 2.00 (br s, 2H), 1.89 (m, 2H), 1.23 (t, J = 7.6 Hz, 3H), 0.98 (d, J = 6.5 Hz, 6H). | 513.3 |
| 30 | 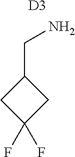 | 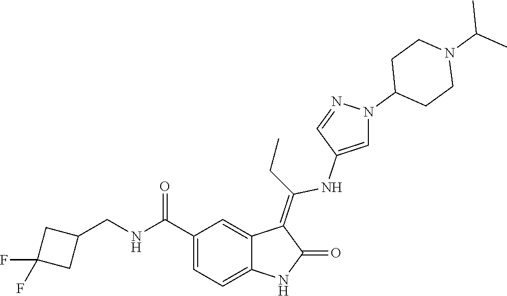 [SMILES: CC/C(Nc1cnn(C2CCN(C(C)C)CC2)c1)=C1/C(=O)Nc2ccc(C(=O)NCC3CC(F)(F)C3)cc21] | L | 23 | 11.69 (s, 1H), 10.81 (s, 1H), 8.47 (t, J = 5.9 Hz, 1H), 8.03 (s, 1H), 7.74 (d, J = 1.6 Hz, 1H), 7.56 (s, 1H), 7.53 (dd, J = 8.1, 1.5 Hz, 1H), 6.91 (d, J = 8.1 Hz, 1H), 4.09 (m, 1H), 3.36 (t, J = 5.8 Hz, 2H), 2.87 (m, 2H), 2.79 (q, J = 7.6 Hz, 2H), 2.73 (m, 1H), 2.61 (m, 2H), 2.31-2.44 (m, 3H), 2.24 (m, 2H), 2.00 (m, 2H), 1.89 (m, 2H), 1.23 (t, J = 7.5 Hz, 3H), 0.97 (d, J = 6.5 Hz, 6H). | 527.2 |
| 31 | 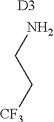 | 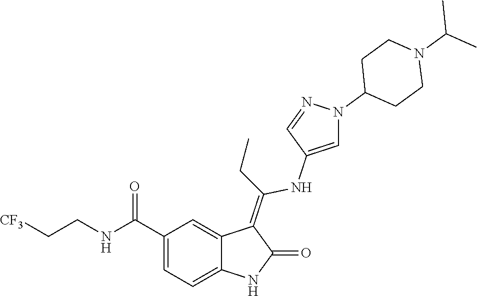 [SMILES: CC/C(Nc1cnn(C2CCN(C(C)C)CC2)c1)=C1/C(=O)Nc2ccc(C(=O)NCCC(F)(F)F)cc21] | L (T3P) | 46 | 11.70 (s, 1H), 10.82 (s, 1H), 8.53 (br s, 1H), 8.03 (s, 1H), 7.74 (s, 1H), 7.56 (s, 1H), 7.52 (dd, J = 8.2, 1.5 Hz, 1H), 6.92 (d, J = 8.1 Hz, 1H), 4.10 (m, 1H), 3.47 (q, J = 6.6 Hz, 2H), 2.87 (m, 2H), 2.69-2.83 (m, 3H), 2.55 (m, 2H), 2.25 (m, 2H), 2.01 (m, 2H), 1.90 (m, 2H), 1.23 (t, J = 7.6 Hz, 3H), 0.98 (d, J = 6.5 Hz, 6H). | 519.2 |
| 32 | 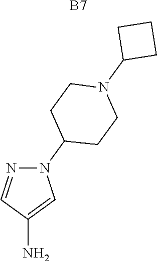 | 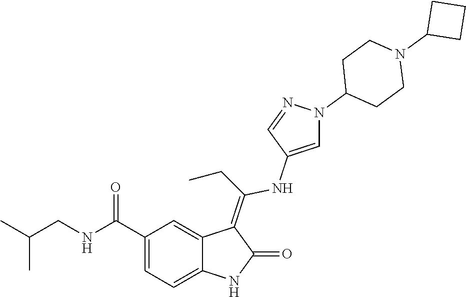 [SMILES: CC/C(Nc1cnn(C2CCN(C3CCC3)CC2)c1)=C1/C(=O)Nc2ccc(C(=O)NCC(C)C)cc21] | K | 24 | 11.68 (s, 1H), 10.50 (s, 1H), 7.99 (br t, J = 5.6 Hz, 1H), 7.94 (s, 1H), 7.76 (s, 1H), 7.54 (d, J = 1.2 Hz, 1H), 7.53 (s, 1H), 6.92 (d, J = 8.0 Hz, 1H), 4.16 (br m, 1H), 3.10 (m, 2H), 2.91 (br m, 2H), 2.84 (q, J = 7.6 Hz, 2H), 1.94-2.03 (br m, 8H), 1.77-1.92 (m, 4H), 1.62-1.69 (m, 2H), 1.25 (t, J = 7.6 Hz, 3H), 0.91 (d, J = 6.4 Hz, 6H), at high temp. | 491.3 |
| 33 | 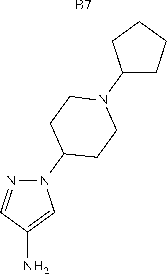 | 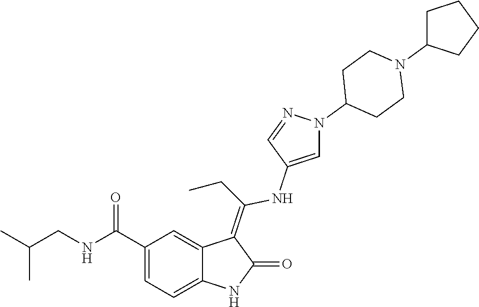 [SMILES: CC/C(Nc1cnn(C2CCN(C3CCCC3)CC2)c1)=C1/C(=O)Nc2ccc(C(=O)NCC(C)C)cc21] | K | 31 | 11.70 (s, 1H), 10.76 (s, 1H), 8.29 (t, J = 6.0 Hz, 1H), 8.02 (s, 1H), 7.75 (s, 1H), 7.53-7.57 (m, 2H), 6.90 (d, J = 8.0 Hz, 1H), 4.13 (m, 1H), 3.00-3.08 (m, 4H), 2.80 (q, J = 7.6 Hz, 2H), 1.90-2.10 (m, 6H), 1.75-1.89 (m 3H), 1.61 (m, 2H), 1.52 (m, 2H), 1.34 (m, 2H), 1.23 (t, J = 7.6 Hz, 3H), 0.88 (d, J = 6.8 Hz, 6H), one H is under solvents. | 505.5 |
| 34 | 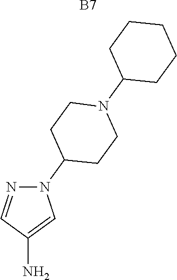 | 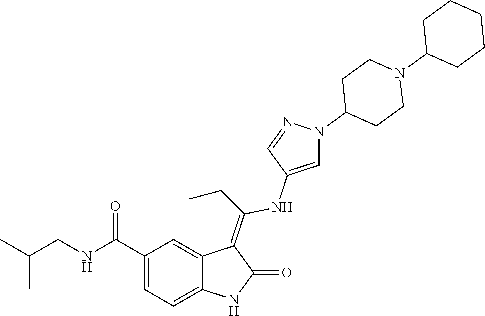 [SMILES: CC/C(Nc1cnn(C2CCN(C3CCCCC3)CC2)c1)=C1/C(=O)Nc2ccc(C(=O)NCC(C)C)cc21] | K | 27 | 11.69 (s, 1H), 10.76 (s, 1H), 8.29 (t, J = 6.0 Hz, 1H), 8.02 (s, 1H), 7.75 (s, 1H), 7.56 (s, 1H), 7.54 (d, J = 8.8 Hz, 1H), 6.90 (d, J = 8.0 Hz, 1H), 4.09 (m, 1H), 3.06 (dd, J = 6.4. 6.4 Hz, 2H), 2.92 (br m, 2H), 2.81 (q, J = 7.2 Hz, 2H), 2.28-2.40 (br m, 3H), 2.00 (br m, 2H), 1.80-1.94 (m, 3H), 1.70-1.80 (br m, 4H), 1.57 (br m, 1H), 1.15- 1.25 (m, 7H), 1.03- 1.10 (m, 1H), 0.88 (d, J = 6.8 Hz, 6H). | 519.5 |
| 35 | 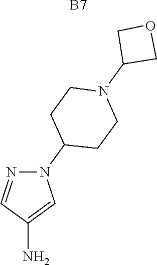 | 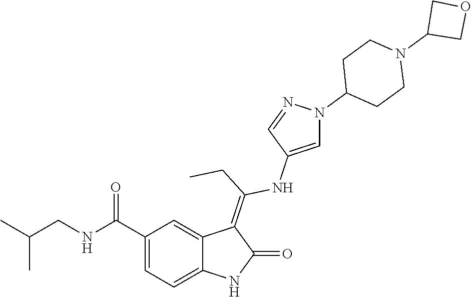 [SMILES: CC/C(Nc1cnn(C2CCN(C3COC3)CC2)c1)=C1/C(=O)Nc2ccc(C(=O)NCC(C)C)cc21] | K | 24 | 11.70 (s, 1H), 10.77 (s, 1H), 8.30 (t, J = 5.6 Hz, 1H), 8.04 (s, 1H), 7.75 (s, 1H), 7.58 (s, 1H), 7.54 (d, J = 8.0 Hz, 1H), 6.91 (d, J = 8.4 Hz, 1H), 4.54 (t, J = 6.4 Hz, 2H), 4.44 (t, J = 6.4 Hz, 2H), 4.17 (br m, 1H), 3.42 (m, 1H), 3.06 (m, 2H), 2.82 (br m, 4H), 1.90-2.08 (m, 6H), 1.79-1.89 (m, 1H), 1.24 (t, J = 7.6 Hz, 3H), 0.88 (d, J = 6.8 Hz, 6H). | 493.4 |
| 36 | 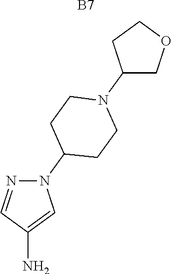 | 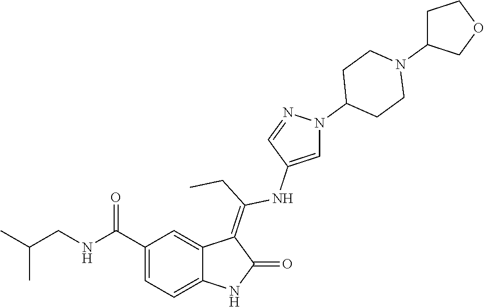 [SMILES: CC/C(Nc1cnn(C2CCN(C3CCOC3)CC2)c1)=C1/C(=O)Nc2ccc(C(=O)NCC(C)C)cc21] | K | 25 | 11.69 (s, 1H), 10.77 (s, 1H), 8.29 (t, J = 6.0 Hz, 1H), 8.02 (s, 1H), 7.75 (s, 1H), 7.56 (s, 1H), 7.54 (dd, J = 8.0, 0.8 Hz, 1H), 6.90 (d, J = 8.4 Hz, 1H), 4.13 (m, 1H), 3.75-3.82 (m, 2H), 3.63 (q, J = 8.0 Hz, 1H), 3.48 (m, 1H), 3.06 (t, J = 6.0 Hz, 2H), 2.89-2.99 (m, 2H), 2.77-2.83 (br m, 3H), 2.13 (m, 2H), 1.92- 2.02 (m, 5H), 1.85 (m, 1H), 1.75 (m, 1H), 1.23 (t, J = 7.2 Hz, 3H), 0.88 (d, J = 6.4 Hz, 6H). | 507.3 |
| 37 | 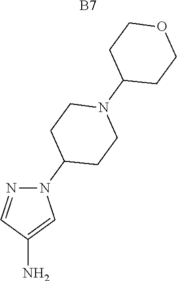 | 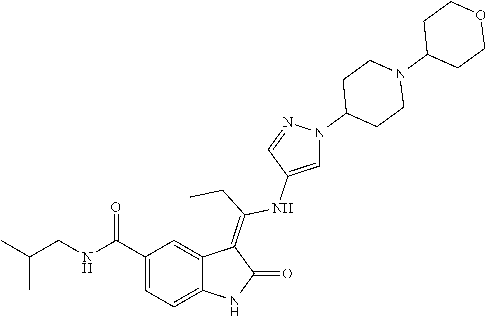 [SMILES: CC/C(Nc1cnn(C2CCN(C3CCOCC3)CC2)c1)=C1/C(=O)Nc2ccc(C(=O)NCC(C)C)cc21] | K | 24 | 11.69 (s, 1H), 10.77 (s, 1H), 8.30 (t, J = 5.6 Hz, 1H), 8.02 (s, 1H), 7.75 (s, 1H), 7.56 (s, 1H), 7.54 (dd, J = 8.0, 1.2 Hz, 1H), 6.90 (d, J = 8.0 Hz, 1H), 4.12 (m, 1H), 3.88 (dd, J = 14.0, 6.8 Hz, 2H), 3.27 (t, J = 11.2 Hz, 2H), 3.06 (t, J = 6.4 Hz, 2H), 2.98 (br m, 2H), 2.10 (q, J = 7.6 Hz, 2H), 2.45 (m, 1H), 2.26 (t, J = 11.6 Hz, 2H), 2.02 (m, 2H), 1.91 (m, 2H), 1.83 (m, 1H), 1.65-1.73 (br m, 2H), 1.44 (m, 2H), 1.23 (t, J = 7.6 Hz, 3H), 0.88 (d, J = 6.4 Hz, 6H). | 521.6 |
| 38 | 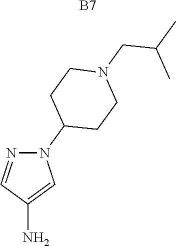 | 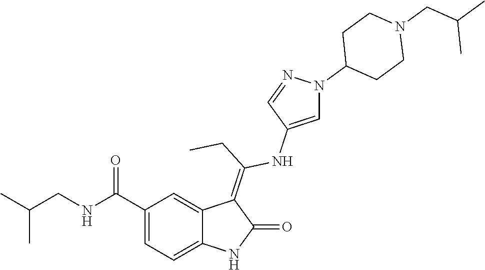 [SMILES: CC/C(Nc1cnn(C2CCN(CC(C)C)CC2)c1)=C1/C(=O)Nc2ccc(C(=O)NCC(C)C)cc21] | K | 24 | 11.70 (s, 1H), 10.76 (s, 1H), 8.29 (t, J = 5.6 Hz, 1H), 8.03 (s, 1H), 7.75 (s, 1H), 7.56 (s, 1H), 7.54 (d, J = 8.4 Hz, 1H), 6.90 (d, J = 8.0 Hz, 1H), 4.12 (m, 1H), 3.06 (t, J = 6.4 Hz, 2H), 2.90 (br d, J = 10.0 Hz, 2H), 2.81 (q, J = 7.2 Hz, 2H), 2.06 (br d, J = 7.6 Hz, 2H), 1.96-2.02 (m, 5H), 1.88-1.95 (m, 1H), 1.84 (m, 1H), 1.76 (m, 1H), 1.23 (br m, 3H), 0.87 (m, 12H). | 493.5 |
| 39 | 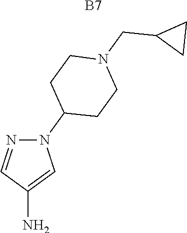 | 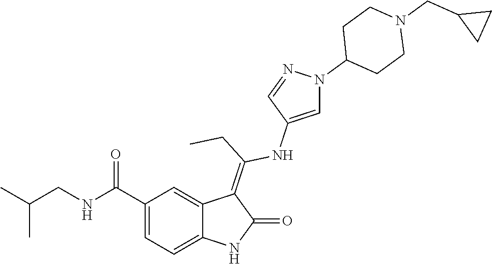 [SMILES: CC/C(Nc1cnn(C2CCN(CC3CC3)CC2)c1)=C1/C(=O)Nc2ccc(C(=O)NCC(C)C)cc21] | K | 32 | 11.70 (s, 1H), 10.77 (s, 1H), 8.30 (t, J = 5.6 Hz, 1H), 8.03 (s, 1H), 7.75 (s, 1H), 7.57 (s, 1H), 7.54 (dd, J = 8.4, 1.2 Hz, 1H), 6.90 (d, J = 8.0 Hz, 1H), 4.14 (br m, 1H), 3.06 (m, 4H), 2.81 (q, J = 7.2 Hz, 2H), 2.21 (br m, 2H), 2.01-2.08 (br m, 6H), 1.84 (br m, 1H), 1.24 (t, J = 7.2 Hz, 3H), 0.88 (d, J = 6.8 Hz, 6H), 0.85 (br m, 1H), 0.48 (br m, 2H), 0.09 (br m, 2H). | 491.3 |
| 40 | 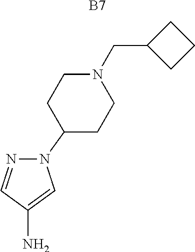 | 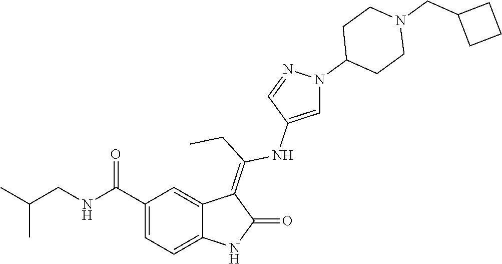 [SMILES: CC/C(Nc1cnn(C2CCN(CC3CCC3)CC2)c1)=C1/C(=O)Nc2ccc(C(=O)NCC(C)C)cc21] | K | 26 | 11.69 (s, 1H), 10.77 (s, 1H), 8.29 (br m, 1H), 8.02 (s, 1H), 7.75 (s, 1H), 7.52-7.58 (m, 2H), 6.90 (br d, J = 7.6 Hz, 1H), 4.10 (m, 1H), 3.06 (br t, J = 6.0 Hz, 2H), 2.88 (br d, J = 9.2 Hz, 2H), 2.80 (br m, 2H), 2.35 (m, 2H), 1.90-2.09 (m, 8H), 1.71-1.90 (m, 4H), 1.58-1.69 (m, 2H), 1.23 (br t, J = 7.6 Hz, 3H), 0.88 (d, J = 6.4 Hz, 6H). | 505.5 |
| 41 | 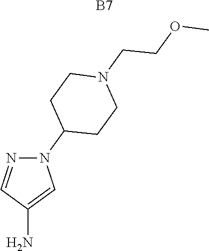 | 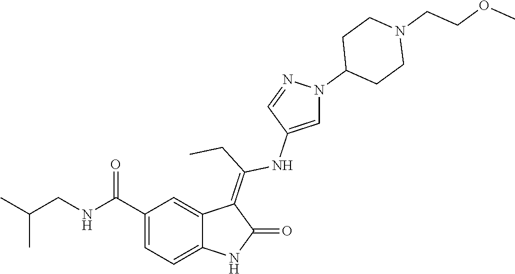 [SMILES: CC/C(Nc1cnn(C2CCN(CCOC)CC2)c1)=C1/C(=O)Nc2ccc(C(=O)NCC(C)C)cc21] | K | 24 | 11.70 (s, 1H), 10.76 (s, 1H), 8.29 (t, J = 5.6 Hz, 1H), 8.02 (s, 1H), 7.75 (s, 1H), 7.57 (s, 1H), 7.54 (d, J = 8.8 Hz, 1H), 6.90 (d, J = 8.0 Hz, 1H), 4.13 (br m, 1H), 3.45 (br m, 2H), 3.24 (s, 3H), 3.06 (t, J = 6.4 Hz, 2H), 2.97 (br m, 2H), 2.80 (q, J = 7.2 Hz, 2H), 2.50 (br m, 2H), 2.06-2.19 (br m, 2H), 1.90-2.03 (br m, 4H), 1.83 (m, 1H), 1.23 (t, J = 7.2 Hz, 3H), 0.88 (d, J = 6.8 Hz, 6H). | 495.5 |
| 42 | 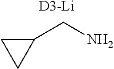 | 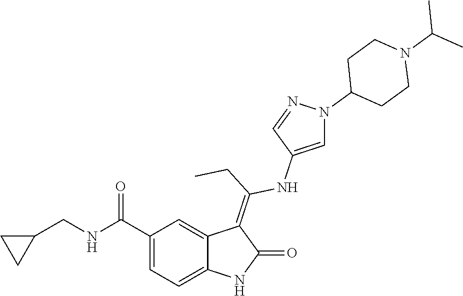 [SMILES: CC/C(Nc1cnn(C2CCN(C(C)C)CC2)c1)=C1/C(=O)Nc2ccc(C(=O)NCC3CC3)cc21] | L | 34 | 11.69 (s, 1H), 10.80 (s, 1H), 8.39 (t, J = 5.8 Hz, 1H), 8.03 (s, 1H), 7.75 (d, J = 1.6 Hz, 1H), 7.55 (m, 2H), 6.90 (d, J = 8.1 Hz, 1H), 4.09 (m, 1H), 3.13 (t, J = 6.2 Hz, 2H), 2.87 (m, 2H), 2.79 (q, J = 7.5 Hz, 2H), 2.73 (m, 1H), 2.25 (m, 2H), 2.01 (m, 2H), 1.89 (m, 2H), 1.23 (t, J = 7.6 Hz, 3H), 1.04 (m, 1H), 0.98 (d, J = 6.5 Hz, 6H), 0.35-0.44 (m, 2H), 0.18-0.24 (m, 2H). | 477.4 |
| 43 | 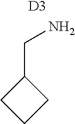 | 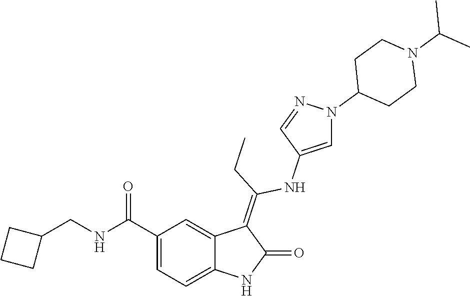 [SMILES: CC/C(Nc1cnn(C2CCN(C(C)C)CC2)c1)=C1/C(=O)Nc2ccc(C(=O)NCC3CCC3)cc21] | L (T3P) | 28 | 11.69 (s, 1H), 10.79 (s, 1H), 8.29 (t, J = 5.9 Hz, 1H), 8.02 (s, 1H), 7.73 (s, 1H), 7.56 (s, 1H), 7.52 (dd, J = 8.2, 1.5 Hz, 1H), 6.89 (d, J = 8.1 Hz, 1H), 4.09 (m, 1H), 3.27 (t, J = 6.4 Hz, 2H), 2.87 (m, 2H), 2.79 (q, J = 7.5 Hz, 2H), 2.73 (m, 1H), 2.53 (m, 1H), 2.24 (m, 2H), 1.76-2.04 (m, 8H), 1.66-1.75 (m, 2H), 1.22 (t, J = 7.6 Hz, 3H), 0.98 (d, J = 6.6 Hz, 6H). | 491.2 |
| 44 | 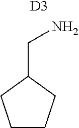 | 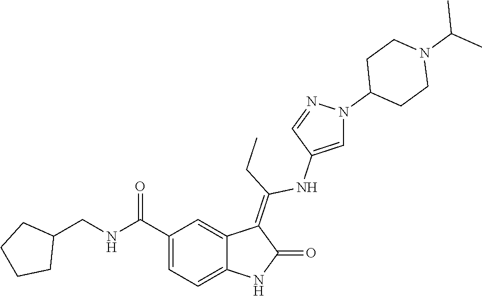 [SMILES: CC/C(Nc1cnn(C2CCN(C(C)C)CC2)c1)=C1/C(=O)Nc2ccc(C(=O)NCC3CCCC3)cc21] | L (T3P) | 44 | 11.69 (s, 1H), 10.79 (s, 1H), 8.32 (t, J = 6.0 Hz, 1H), 8.02 (s, 1H), 7.73 (d, J = 1.5 Hz, 1H), 7.56 (s, 1H), 7.52 (dd, J = 8.1, 1.5 Hz, 1H), 6.90 (d, J = 8.1 Hz, 1H), 4.09 (m, 1H), 3.16 (t, J = 6.5 Hz, 2H), 2.83-2.91 (m, 2H), 2.79 (q, J = 7.6 Hz, 2H), 2.73 (m, 1H), 2.24 (m, 2H), 2.14 (m, 1H), 1.95-2.04 (m, 2H), 1.89 (m, 2H), 1.65 (m, 2H), 1.56 (m, 2H), 1.48 (m, 2H), 1.25 (m, 2H), 1.23 (t, J = 6.1 Hz, 3H), 0.97 (d, J = 6.5 Hz, | 505.4 |
| | | | | | 6H). | |
| 45 | 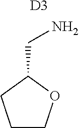 | 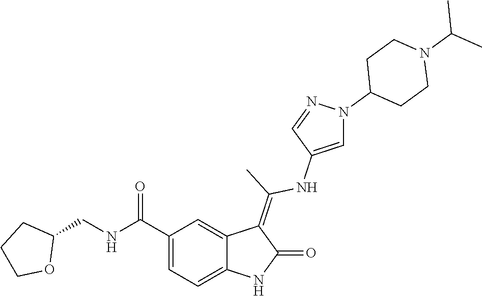 [SMILES: C/C(Nc1cnn(C2CCN(C(C)C)CC2)c1)=C1/C(=O)Nc2ccc(C(=O)NC[C@H]3CCCO3)cc21] | L | 47 | 11.82 (s, 1H), 10.77 (s, 1H), 8.42 (t, J = 6.1 Hz, 1H), 8.01 (s, 1H), 7.84 (d, J = 1.7 Hz, 1H), 7.56 (s, 1H), 7.54 (dd, J = 8.3, 1.8 Hz, 1H), 6.89 (d, J = 8.2 Hz, 1H), 4.07 (m, 1H), 3.96 (m, 1H), 3.76 (m, 1H), 3.61 (q, J = 7.3 Hz, 1H), 3.29 (m, 2H), 2.87 (m, 2H), 2.73 (m, 1H), 2.52 (s, 3H), 2.24 (m, 2H), 2.01 (m, 2H), 1.73-1.94 (m, 5H), 1.59 (m, 1H), 0.97 (d, J = 6.5 Hz, 6H). | 493.3 |
| 46 | 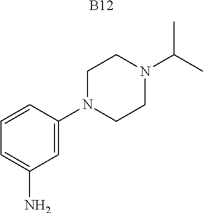 | 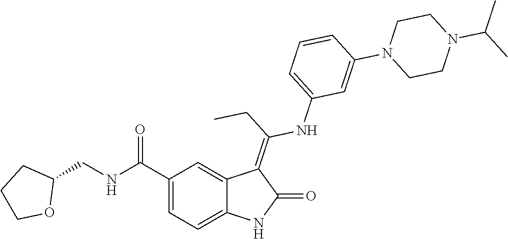 [SMILES: CC/C(Nc1cccc(N2CCN(C(C)C)CC2)c1)=C1/C(=O)Nc2ccc(C(=O)NC[C@H]3CCCO3)cc21] | K | 70 | 12.11 (s, 1H), 10.83 (s, 1H), 8.39 (br t, J = 6.1 Hz, 1H), 7.76 (s, 1H), 7.56 (dd, J = 8.1, 1.4 Hz, 1H), 7.28 (dd (apparent triplet), J = 8.0, 8.0 Hz, 1H), 6.91 (m, 2H), 6.80 (m, 1H), 6.70 (dd, J = 7.7, 1.8 Hz, 1H), 3.96 (m, 1H), 3.76 (m, 1H), 3.61 (m, 1H), 3.23-3.32 (m, 2H), 3.16 (br m, 4H), 2.77-2.92 (m, 2H), 2.65 (m, 1H), 2.56 (br | 518.3 |
| | | | | | m, 4H), 1.73-1.92 (m, | |
| | | | | | 3H), 1.58 (m, 1H), 1.25 | |
| | | | | | (t, J = 7.5 Hz, 3H), 0.99 | |
| | | | | | (d, J = 6.5 Hz, 6H). | |
| 47 | 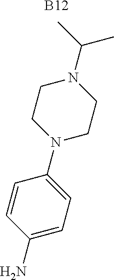 | 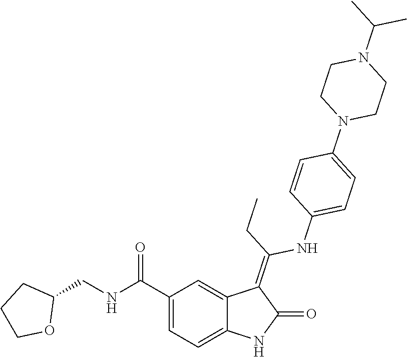 [SMILES: CC/C(Nc1ccc(N2CCN(C(C)C)CC2)cc1)=C1/C(=O)Nc2ccc(C(=O)NC[C@H]3CCCO3)cc21] | K | 81 | 11.98 (s, 1H), 10.77 (s, 1H), 8.36 (br t, J = 5.6 Hz, 1H), 7.75 (s, 1H ), 7.55 (d, J = 8.0 Hz, 1H), 7.17 (d, J = 8.8 Hz, 2H), 6.99 (d, J = 8.8 Hz, 2H), 6.91 (d, J = 8.0 Hz, 1H), 3.98 (m, 1H), 3.77 (q, J = 6.8 Hz, 1H), 3.61 (q, J = 7.2 Hz, 1H), 3.28-3.32 (m, 2H), 3.16 (m, 4H), 2.72-2.80 (m, 2H), 2.63-2.69 (m, 1H), 2.58 (m, 4H), 1.76- 1.92 (m, 3H), 1.52- 1.62 (m, 1H), 1.22 (t, J = 7.6 Hz, 3H), 1.01 (d, J = 5.6 Hz, 6H). | 518.2 |
| 48 | 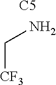 | 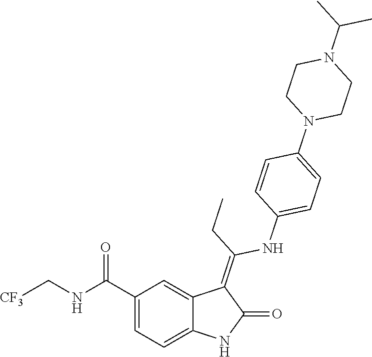 [SMILES: CC/C(Nc1ccc(N2CCN(C(C)C)CC2)cc1)=C1/C(=O)Nc2ccc(C(=O)NCC(F)(F)F)cc21] | I | 18 | 11.98 (s, 1H), 10.84 (s, 1H), 8.87 (t, J = 6.3 Hz, 1H), 7.78 (s, 1H), 7.59 (dd, J = 8.1, 1.6 Hz, 1H), 7.17 (d, J = 8.6 Hz, 2H), 7.00 (d, J = 8.7 Hz, 2H), 6.95 (d, J = 8.2 Hz, 1H), 4.02- 4.09 (m, 2H), 3.17 (m, 4H), 2.78 (q, J = 7.6 Hz, 2H), 2.45 (m, 4H), 2.22 (s, 3H), 1.21 (t, J = 7.4 Hz, 3H). | 488.2 |
| 49 | 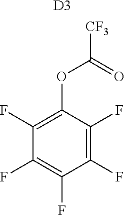 | 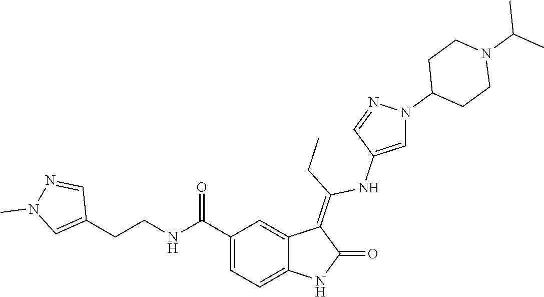 [SMILES: CC/C(Nc1cnn(C2CCN(C(C)C)CC2)c1)=C1/C(=O)Nc2ccc(C(=O)NCCc3cnn(C)c3)cc21] | L | 53 | 11.70 (s, 1H), 10.80 (s, 1H), 8.40 (t, J = 5.6 Hz, 1H), 8.03 (s, 1H), 7.73 (s, 1H), 7.56 (s, 1H), 7.53 (dd, J = 8.1, 1.5 Hz, 1H), 7.50 (s, 1H), 7.26 (s, 1H), 6.90 (d, J = 8.1 Hz, 1H), 4.09 (m, 1H), 3.75 (s, 3H), 3.36 (q, J = 7.0 Hz, 2H), 2.87 (m, 2H), 2.79 (q, J = 7.6 Hz, 2H), 2.73 (m, 1H), 2.63 (t, J = 7.4 Hz, 2H), 2.24 (m, 2H), 2.01 (m, 2H), 1.89 (m, 2H), 1.23 (t, J = 7.6 Hz, 3H), 0.97 (d, J = 6.5 Hz, 6H). | 531.4 |
| | 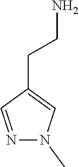 | | | | | |
| 50 | 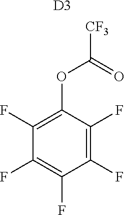 | 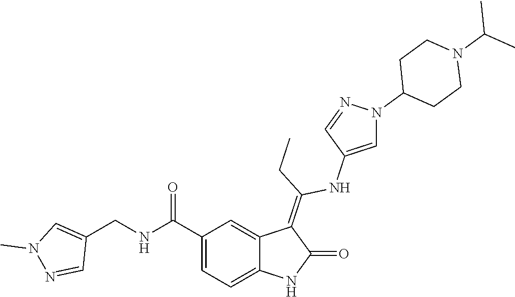 [SMILES: CC/C(Nc1cnn(C2CCN(C(C)C)CC2)c1)=C1/C(=O)Nc2ccc(C(=O)NCc3cnn(C)c3)cc21] | L | 60 | 11.69 (s, 1H), 10.80 (s, 1H), 8.68 (t, J = 5.9 Hz, 1H), 8.02 (s, 1H), 7.76 (d, J = 1.7 Hz, 1H), 7.55 (m, 3H), 7.33 (s, 1H), 6.89 (d, J = 8.2 Hz, 1H), 4.26 (d, J = 5.7 Hz, 2H), 4.09 (m, 1H), 3.76 (s, 3H), 2.86 (m, 2H), 2.79 (q, J = 7.5 Hz, 2H), 2.73 (m, 1H), 2.24 (m, 2H), 2.01 (m, 2H), 1.89 (m, 2H), 1.22 (t, J = 7.6 Hz, 3H), 0.97 (d, J = 6.5 Hz, 6H). | 517.4 |
| | 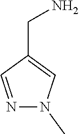 | | | | | |
| 51 | 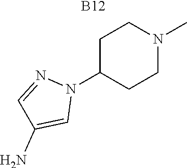 | 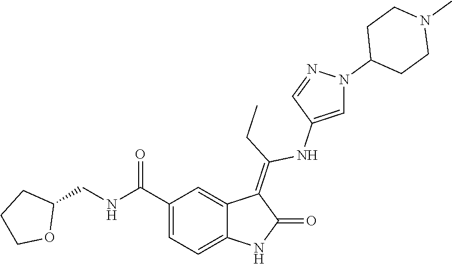 [SMILES: CC/C(Nc1cnn(C2CCN(C)CC2)c1)=C1/C(=O)Nc2ccc(C(=O)NC[C@H]3CCCO3)cc21] | K | 38 | 11.71 (s, 1H), 10.78 (s, 1H), 8.37 (t, J = 5.6 Hz, 1H), 8.03 (s, 1H), 7.76 (s, 1H), 7.58 (s, 1H), 7.55 (d, J = 8.4 Hz, 1H), 6.91 (d, J = 8.0 Hz, 1H), 4.16 (br m, 1H), 3.98 (m, 1H), 3.77 (q, J = 7.0 Hz, 1H), 3.62 (q, J = 7.2 Hz, 1H), 3.26-3.31 (m, 2H), 2.88-3.00 (m, 2H), 2.76-2.85 (m, 2H), 2.29 (br s, 3H), 2.21 (m, 2H), 1.95- 2.09 (br m, 4H), 1.75- 1.87 (m, 3H), 1.59 (m, | 479.5 |
| | | | | | 1H), 1.23 (t, J = 7.6 Hz, | |
| | | | | | 3H). | |
| 52 | 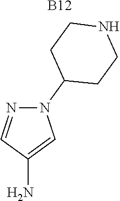 | 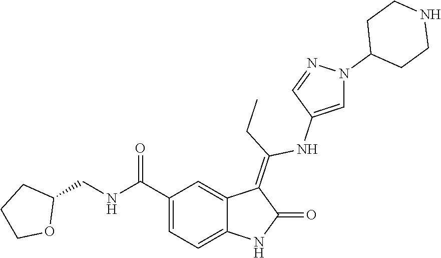 [SMILES: CC/C(Nc1cnn(C2CCNCC2)c1)=C1/C(=O)Nc2ccc(C(=O)NC[C@H]3CCCO3)cc21] | K | 83 | 11.70 (s, 1H), 10.80 (s, 1H), 8.39 (br s, 1H), 8.00 (s, 1H), 7.75 (s, 1H), 7.56 (s, 1H), 7.54 (d, J = 8.1 Hz, 1H), 6.90 (d, J = 8.1 Hz, 1H), 4.19 (m, 1H), 3.96 (m, 1H), 3.76 (q, J = 7.1 Hz, 1H), 3.61 (q, J = 7.3 Hz, 1H), 3.23- 3.33 (m, 2H), 3.04 (m, 2H), 2.74-2.86 (m, 2H), 2.58 (m, 2H), 1.95 (m, 2H), 1.71-1.92 (m, 5H), 1.58 (m, 1H), 1.23 (t, J = 7.5 Hz, 3H), NH | 465.2 |
| | | | | | is under solvent peaks. | |
| 53 | 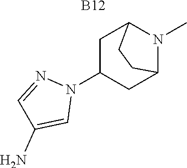 | 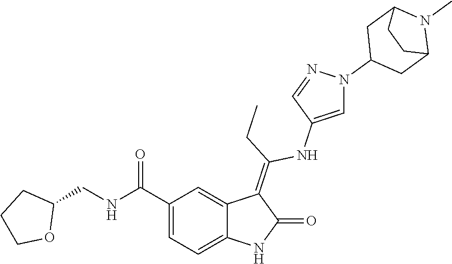 [SMILES: CC/C(Nc1cnn(C2CC3CCC(C2)N3C)c1)=C1/C(=O)Nc2ccc(C(=O)NC[C@H]3CCCO3)cc21] | K | 27 | 11.67 (s, 1H), 10.79 (s, 1H), 8.37 (t, J = 5.6 Hz, 1H), 8.21 (s, 1H), 7.76 (s, 1H), 7.59 (s, 1H), 7.55 (d, J = 8.0 Hz, 1H), 6.90 (d, J = 8.0 Hz, 1H), 4.36 (br m, 1H), 3.98 (br m, 1H), 3.77 (m, 1H), 3.62 (m, 1H), 3.35-3.40 (br m, 2H), 3.25-3.30 (br m, 2H), 3.10-3.20 (br m, 2H), 2.77-2.87 (br m, 2H), 2.26-2.29 (br m, 2H), 2.19 (br s, 3H), 1.75-1.93 (m, 5H), 1.55-1.63 (m, 1H), | 505.5 |
| | | | | | 1.34-144 (br m, 2H), | |
| | | | | | 1.21 (t, J = 7.2 Hz, 3H). | |
| 54 | 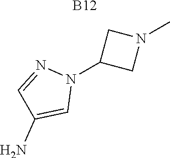 | 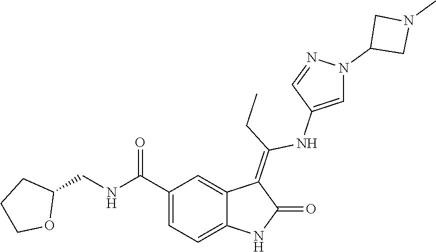 [SMILES: CC/C(Nc1cnn(C2CN(C)C2)c1)=C1/C(=O)Nc2ccc(C(=O)NC[C@H]3CCCO3)cc21] | K | 32 | 11.72 (s, 1H), 10.79 (s, 1H), 8.37 (br t, J = 5.6 Hz, 1H), 8.11 (s, 1H), 7.76 (s, 1H), 7.65 (s, 1H), 7.56 (d, J = 8.0 Hz, 1H), 6.91 (d, J = 8.0 Hz, 1H), 4.95 (m, 1H), 3.96 (m, 1H), 3.76 (q, J = 7.2 Hz, 1H), 3.71 (t, J = 7.2 Hz, 2H), 3.63 (q, J = 6.8 Hz, 1H), 3.39 (t, J = 6.8 Hz, 2H), 3.31 (br m, 1H), 2.81 (br m, 2H), 2.33 (s, 3H), 1.75- 1.95 (m, 4H), 1.60 (m, | 451.4 |
| | | | | | 1H), 1.24 (t, J = 7.2 Hz, | |
| | | | | | 3H). | |
| 55 | 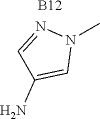 | 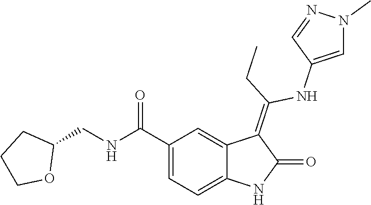 [SMILES: CC/C(Nc1cnn(C)c1)=C1/C(=O)Nc2ccc(C(=O)NC[C@H]3CCCO3)cc21] | K | 41 | 11.68 (s, 1H), 10.79 (s, 1H), 8.38 (t, J = 5.6 Hz, 1H), 7.93 (s, 1H), 7.76 (s, 1H), 7.55 (d, J = 8.0 Hz, 1H), 7.54 (s, 1H), 6.90 (d, J = 8.4 Hz, 1H), 3.97 (m, 1H), 3.86 (s, 3H), 3.77 (m, 1H), 3.61 (m, 1H), 3.25- 3.31 (m, 2H), 2.77- 2.86 (m, 2H), 1.75- 1.93 (m, 3H), 1.55- 1.63 (m, 1H), 1.23 (t, J = | 396.4 |
| | | | | | 7.2 Hz, 3H). | |
| 56 | 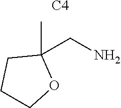 | 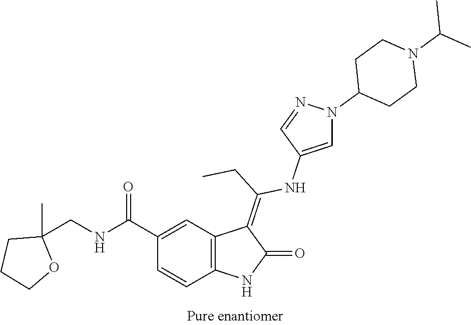 [SMILES: CC/C(Nc1cnn(C2CCN(C(C)C)CC2)c1)=C1/C(=O)Nc2ccc(C(=O)NCC3(C)CCCO3)cc21] | J & chiral separation | 5 | 11.70 (s, 1H), 10.78 (s, 1H), 8.25 (t, J = 6.0 Hz, 1H), 8.03 (s, 1H), 7.76 (s, 1H), 7.60 (br s, 1H), 7.56 (dd, J = 8.0, 1.2 Hz, 1H), 6.90 (d, J = 8.4 Hz, 1H), 4.10-4.40 (br m, 1H), 3.75 (m, 2H), 2.82 (m, 2H), 2.10-2.30 (br m, 4H), 1.83-1.92 (m, 3H), 1.52 (m, 1H), 1.23- 1.33 (m, 6H), 1.04- 1.22 (m, 7H), 6Hs are under solvent peaks. | 521.4 |
| 57 | 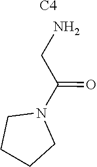 | 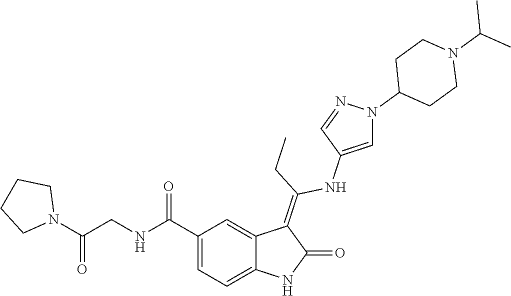 [SMILES: CC/C(Nc1cnn(C2CCN(C(C)C)CC2)c1)=C1/C(=O)Nc2ccc(C(=O)NCC(=O)N3CCCC3)cc21] | J | 8 | 11.71 (s, 1H), 10.81 (s, 1H), 8.43 (t, J = 5.2 Hz, 1H), 8.03 (s, 1H), 7.79 (s, 1H), 7.60 (br s, 1H), 7.58 (d, J = 8.0 Hz, 1H), 6.95 (d, J = 8.4 Hz, 1H), 4.05-4.20 (br m, 1H), 4.00 (d, J = 5.6 Hz, 2H), 3.47 (t, J = 6.8 Hz, 2H), 3.30 (br m, 4H), 2.92-3.10 (br m, 2H), 2.82 (q, J = 7.2 Hz, 2H), 2.43-2.50 (br m, 1H), 2.07-2.19 (br m, 4H), 1.91 (m, 2H), 1.77 (m, 2H), 1.22 (t, J = 7.6 Hz, 3H), 1.06- 1.14 (br m, 6H). | 534.5 |
| 58 | 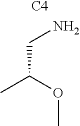 | 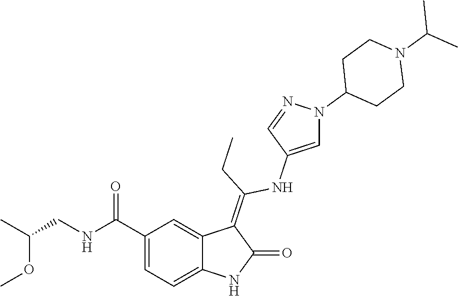 [SMILES: CC/C(Nc1cnn(C2CCN(C(C)C)CC2)c1)=C1/C(=O)Nc2ccc(C(=O)NC[C@@H](C)OC)cc21] | J | 43 | 11.7 (s, 1H), 10.7 (s, 1H), 8.34 (t, J = 5.6 Hz, 1H), 8.02 (s, 1H), 7.76 (s, 1H), 7.56 (s, 1H), 7.55 (d, J = 8.4 Hz, 1H), 6.91 (d, J = 8.4 Hz, 1H), 4.10 (br m, 1H), 3.48 (m, 1H), 3.27 (s, 3H), 3.20-3.32 (m, 2H), 2.87 (br d, J = 8.8 Hz, 2H), 2.81 (br q, J = 7.2 Hz, 2H), 2.73 (m, 1H), 2.25 (br t, J = 10.0 Hz, 2H), 1.98-2.08 (m, 2H), 1.83-1.96 (m, 2H), 1.23 (t, J = 7.2 Hz, 3H), 1.08 (d, J = 6.4 Hz, 3H), 0.99 (br d, J = 6.4 Hz, 6H). | 495.5 |
| 59 | 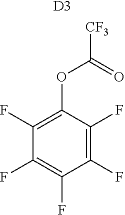 | 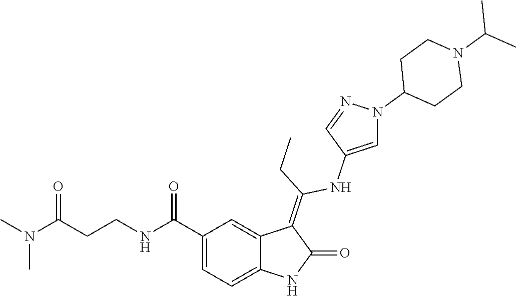 [SMILES: CC/C(Nc1cnn(C2CCN(C(C)C)CC2)c1)=C1/C(=O)Nc2ccc(C(=O)NCCC(=O)N(C)C)cc21] | L | 30 | 11.69 (s, 1H), 10.80 (d, J = 2.3 Hz, 1H), 8.34 (t, J = 6.2 Hz, 1H), 8.02 (s, 1H), 7.74 (d, J = 1.6 Hz, 1H), 7.56 (s, 1H), 7.52 (dd, J = 8.2, 1.5 Hz, 1H), 6.90 (d, J = 8.1 Hz, 1H), 4.09 (m, 1H), 3.43 (q, J = 6.8 Hz, 2H), 2.95 (s, 3H), 2.87 (m, 2H), 2.81 (s, 3H), 2.78 (q, J = 7.6 Hz, 2H), 2.73 (m, 1H), 2.56 (t, J = 7.3 Hz, 2H), 2.24 (m, 2H), 2.01 (m, 2H), 1.89 (m, 2H), 1.23 (t, J = 7.6 Hz, 3H), 0.97 (d, J = 6.6 Hz, 6H). | 522.4 |
| | 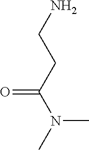 | | | | | |
| 60 | 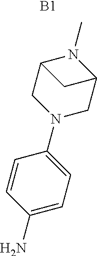 | 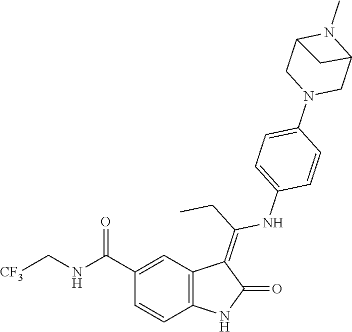 [SMILES: CC/C(Nc1ccc(N2CC3CC(C2)N3C)cc1)=C1/C(=O)Nc2ccc(C(=O)NCC(F)(F)F)cc21] | K | 30 | 11.94 (s, 1H), 10.84 (s, 1H), 8.88 (t, J = 6.0 Hz, 1H), 7.79 (s, 1H), 7.59 (d, J = 7.6 Hz, 1H), 7.20 (d, J = 8.4 Hz, 2H), 6.95 (d, J = 8.4 Hz, 1H), 6.80 (d, J = 8.8 Hz, 2H), 4.02-4.11 (m, 2H), 3.62 (br m, 2H), 3.49 (br m, 2H), 3.32 (br m, 2H), 2.78 (q, J = 7.2 Hz, 2H), 2.50 (m, 1H), 2.48 (m, 2H), 2.03 (s, 3H), 1.56 (br m, 1H), 1.22 (t, J = 7.2 Hz, 3H) in pyridine-d₅. | 500.4 |
| 61 | 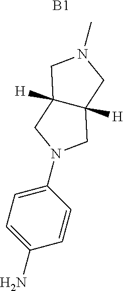 | 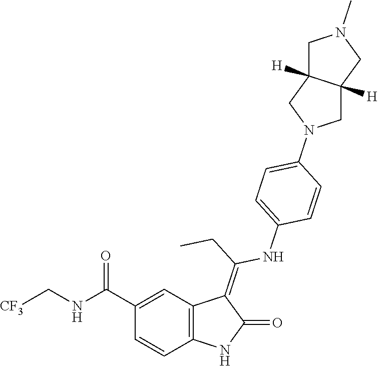 [SMILES: CC/C(Nc1ccc(N2C[C@H]3CN(C)C[C@H]3C2)cc1)=C1/C(=O)Nc2ccc(C(=O)NCC(F)(F)F)cc21] | K | 20 | 11.94 (s, 1H), 10.83 (s, 1H), 8.87 (t, J = 6.0 Hz, 1H), 7.78 (s, 1H), 7.59 (d, J = 8.4 Hz, 1H), 7.15 (d, J = 8.4 Hz, 2H), 6.95 (d, J = 8.0 Hz, 1H), 6.70 (d, J = 8.4 Hz, 2H), 4.02-4.11 (m, 2H), 3.36 (m, 4H), 3.18 (br m, 2H), 2.95 (br m, 2H), 2.77 (q, J = 7.6 Hz, 2H), 2.55 (m, 2H), 2.31 (s, 3H), 1.20 (t, J = 7.6 Hz, 3H). | 514.4 |
| 62 | 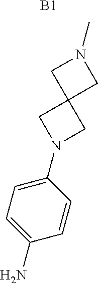 | 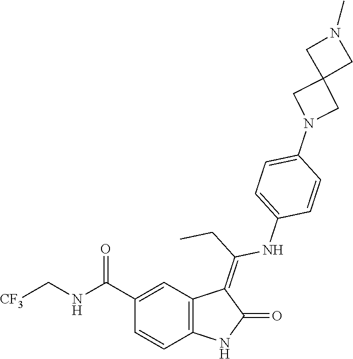 [SMILES: CC/C(Nc1ccc(N2CC3(CN(C)C3)C2)cc1)=C1/C(=O)Nc2ccc(C(=O)NCC(F)(F)F)cc21] | K | 17 | 11.93 (s, 1H), 10.82 (s, 1H), 8.87 (br t, J = 6.0 Hz, 1H), 7.78 (br s, 1H), 7.58 (d, J = 8.0 Hz, 1H), 7.14 (d, J = 8.4 Hz, 2H), 6.94 (d, J = 8.0 Hz, 1H), 6.47 (d, J = 8.8 Hz, 2H), 4.02 - 4.11 (m, 2H), 3.90 (s, 4H), 3.30 (s, 4H), 2.75 (br q, J = 7.6 Hz, 2H), 2.19 (s, 3H), 1.95 (t, J = 7.6 Hz, 3H). | 500.4 |
| 63 | 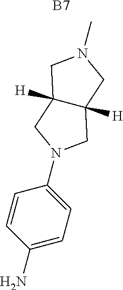 | 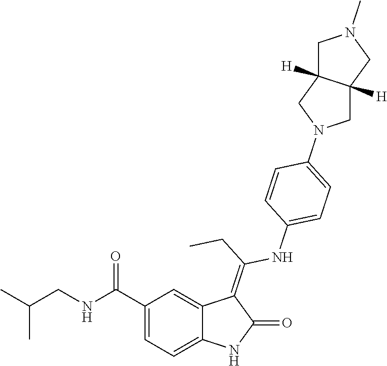 [SMILES: CC/C(Nc1ccc(N2C[C@H]3CN(C)C[C@H]3C2)cc1)=C1/C(=O)Nc2ccc(C(=O)NCC(C)C)cc21] | K | 24 | 11.92 (s, 1H), 10.74 (s, 1H), 8.28 (br t, J = 5.6 Hz, 1H), 7.73 (s, 1H), 7.54 (d, J = 8.0 Hz, 1H), 7.14 (d, J = 8.4 Hz, 2H), 6.91 (d, J = 8.0 Hz, 1H), 6.70 (d, J = 8.4 Hz, 2H), 3.39 (m, 2H), 3.12 (dd, J = 9.2, 2.0 Hz, 2H), 3.06 (br t, J = 6.4 Hz, 2H), 2.91 (br m, 2H), 2.76 (q, J = 7.2 Hz, 2H), 2.59 (br m, 2H), 2.44 (br m, 2H), 2.23 (s, 3H), 1.83 (m, 1H), 1.20 (t, J = 7.6 Hz, 3H), 0.88 (d, J = 6.8 Hz, 6H). | 488.2 |
| 64 | 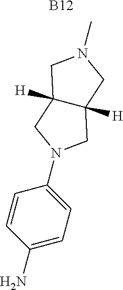 | 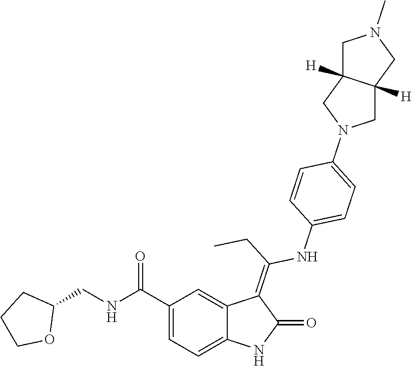 [SMILES: CC/C(Nc1ccc(N2C[C@H]3CN(C)C[C@H]3C2)cc1)=C1/C(=O)Nc2ccc(C(=O)NC[C@H]3CCCO3)cc21] | K | 44 | 7.72(s, 1H), 7.51 (d, J = 8.0 Hz, 1H), 7.11 (d, J = 8.8 Hz, 2H), 6.95 (d, J = 8.4 Hz, 1H), 6.71 (d, J = 8.4 Hz, 2H), 3.95 (m, 1H), 3.73 (m, 2H), 3.60 (q, J = 6.4 Hz, 1H), 3.25-3.35 (m, 4H), 3.12-3.19 (m, 2H), 2.95 (br m, 2H), 2.77 (br m, 4H), 2.28 (s, 3H), 1.72-1.90 (br m, 4H), 1.55 (m, 1H), 1.19 (t, J = 7.6 Hz, 3H) in DMSO-d₆ + D₂O, 3NHs are under solvent peaks. | 516.6 |
| 65 | 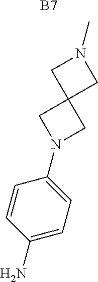 | 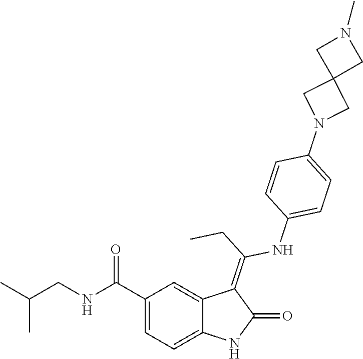 [SMILES: CC/C(Nc1ccc(N2CC3(CN(C)C3)C2)cc1)=C1/C(=O)Nc2ccc(C(=O)NCC(C)C)cc21] | K | 6 | 11.94 (s, 1H), 10.74 (s, 1H), 8.28 (br t, J = 5.6 Hz, 1H), 7.73 (s, 1H), 7.52 (d, J = 8.0 Hz, 1H), 7.13 (d, J = 8.4 Hz, 2H), 6.89 (d, J = 8.4 Hz, 1H), 6.47 (d, J = 8.4 Hz, 2H), 3.89 (s, 4H), 3.26 (s, 4H), 3.06 (t, J = 6.0 Hz, 2H), 2.73 (q, J = 7.6 Hz, 2H), 2.18 (s, 3H), 1.83 (m, 1H), 1.19 (t, J = 7.6 Hz, 3H), 0.87 (d, J = 6.8 Hz, 6H). | 474.5 |
| 66 | 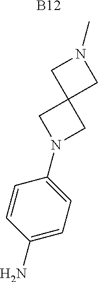 | 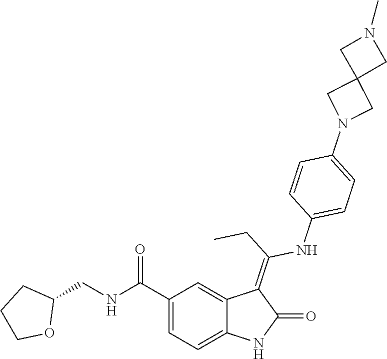 [SMILES: CC/C(Nc1ccc(N2CC3(CN(C)C3)C2)cc1)=C1/C(=O)Nc2ccc(C(=O)NC[C@H]3CCCO3)cc21] | K | 14 | 11.91 (s, 1H), 10.74 (s, 1H), 8.34 (t, J = 6.0 Hz, 1H), 7.74 (s, 1H), 7.54 (dd, J = 8.0, 1.6 Hz, 1H), 7.12 (d, J = 8.8 Hz, 2H), 6.88 (d, J = 8.0 Hz, 1H), 6.48 (d, J = 8.4 Hz, 2H), 3.97 (m, 1H), 3.89 (s, 4H), 3.77 (m, 1H), 3.61 (m, 1H), 3.26 (s, 4H), 2.72-2.77 (br m, 2H) 2.18 (s, 3H), 1.84-1.92 (m, 1H), 1.75-1.83 (m, 2H), 1.55-1.61 (m, 1H), 1.19 (t, J = 7.6 Hz, 3H), 2Hs are under solvent peaks. | 502.5 |
| 67 | 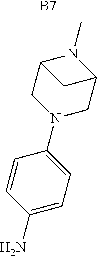 | 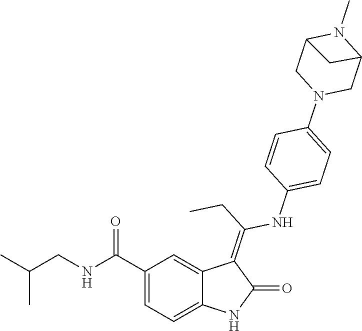 [SMILES: CC/C(Nc1ccc(N2CC3CC(C2)N3C)cc1)=C1/C(=O)Nc2ccc(C(=O)NCC(C)C)cc21] | K | 20 | 11.93 (s, 1H), 10.48 (s, 1H), 7.92 (br m, 1H), 7.76 (s, 1H), 7.52 (d, J = 8.0 Hz, 1H), 7.18 (d, J = 8.4 Hz, 2H), 6.91 (d, J = 8.4 Hz, 1H), 6.82 (d, J = 8.4 Hz, 2H), 3.72-3.86 (br m, 2H), 3.52-3.61 (br m, 2H), 3.40-3.49 (br m, 2H), 3.06-3.12 (m, 2H), 2.82 (q, J = 7.6 Hz, 2H), 2.23-2.30 (br m, 3H), 1.84-1.91 (m, 2H), 1.66 (br m, 1H), 1.24 (t, J = 7.6 Hz, 3H), 0.92 (d, J = 6.8 Hz, 6H), at high temp. | 474.4 |
| 68 | 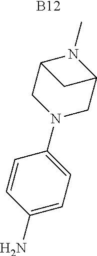 | 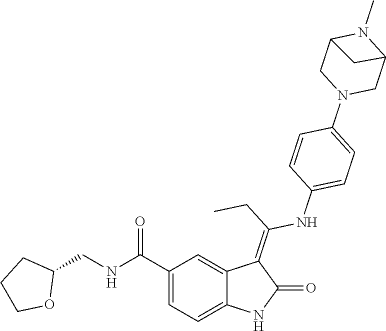 [SMILES: CC/C(Nc1ccc(N2CC3CC(C2)N3C)cc1)=C1/C(=O)Nc2ccc(C(=O)NC[C@H]3CCCO3)cc21] | K | 19 | 11.91 (s, 1H), 10.74 (s, 1H), 8.34 (t, J = 6.0 Hz, 1H), 7.74 (s, 1H), 7.54 (dd, J = 8.0, 1.6 Hz, 1H), 7.12 (d, J = 8.8 Hz, 2H), 6.88 (d, J = 8.0 Hz, 1H), 6.48 (d, J = 8.4 Hz, 2H), 3.97 (m, 1H), 3.89 (s, 4H), 3.77 (m, 1H), 3.61 (m, 1H), 3.26 (s, 4H), 2.72-2.77 (br m, 2H) 2.18 (s, 3H), 1.84-1.92 (m, 1H), 1.75-1.83 (m, 2H), 1.55-1.61 (m, 1H), 1.19 (t, J = 7.6 Hz, 3H), 2Hs are under solvent peaks. | 502.5 |
| 69 | 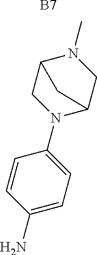 | 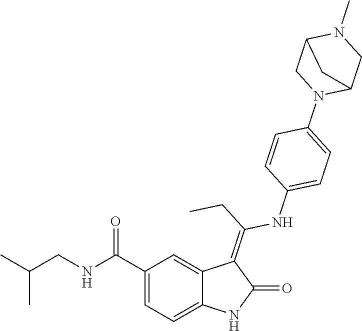 [SMILES: CC/C(Nc1ccc(N2CC3CC2CN3C)cc1)=C1/C(=O)Nc2ccc(C(=O)NCC(C)C)cc21] | K | 25 | 11.91 (s, 1H), 10.47 (s, 1H), 7.98 (br m, 1H), 7.76 (s, 1H), 7.53 (br d, J = 7.6 Hz, 1H), 7.11 (d, J = 8.0 Hz, 2H), 6.92 (d, J = 8.0 Hz, 1H), 6.66 (d, J = 8.0 Hz, 2H), 4.38 (br s, 1H), 3.50-3.65 (br m, 1H), 3.37 (br m, 1H), 3.20 (m, 1H), 3.06 (m, 2H), 2.87 (m, 1H), 2.80 (q, J = 6.4 Hz, 2H), 2.74 (br m, 1H), 2.41 (s, 3H), 1.97 (m, 1H), 1.81-1.87 (m, 2H), 1.21 (t, J = 6.8 Hz, 3H), 0.88 (d, J = 6.4 Hz, 6H), at high temp. | 474.4 |
| 70 | 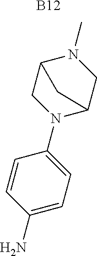 | 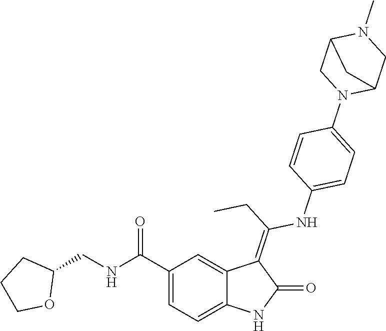 [SMILES: CC/C(Nc1ccc(N2CC3CC2CN3C)cc1)=C1/C(=O)Nc2ccc(C(=O)NC[C@H]3CCCO3)cc21] | K | 29 | 11.92 (s, 1H), 10.75 (s, 1H), 8.36 (t, J = 6.0 Hz, 1H), 7.74 (s, 1H), 7.54 (dd, J = 8.4, 1.2 Hz, 1H), 7.10 (d, J = 8.8 Hz, 2H), 6.90 (d, J = 8.0 Hz, 1H), 6.63 (d, J = 8.8 Hz, 2H), 4.32 (br s, 1H), 3.96 (m, 1H), 3.77 (m, 1H), 3.61 (m, 1H), 3.42 (br s, 1H), 3.24-3.33 (br m, 4H), 3.16 (m, 1H), 2.74- 2.81 (m, 3H), 2.26 (s, 3H), 1.73-1.92 (m, 5H), 1.58 (m, 1H), 1.21 (t, J = 7.6 Hz, 3H). | 502.5 |
| 71 | 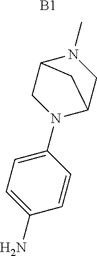 | 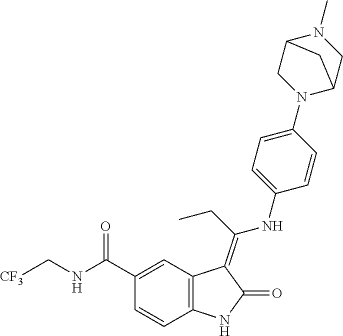 [SMILES: CC/C(Nc1ccc(N2CC3CC2CN3C)cc1)=C1/C(=O)Nc2ccc(C(=O)NCC(F)(F)F)cc21] | K | 29 | 11.94 (s, 1H), 10.83 (s, 1H), 8.88 (t, J = 6.4 Hz, 1H), 7.78 (s, 1H), 7.58 (d, J = 8.0 Hz, 1H), 7.10 (d, J = 8.4 Hz, 2H), 6.94 (d, J = 8.0 Hz, 1H), 6.65 (d, J = 8.8 Hz, 2H), 4.33 (br s, 1H) 4.02-4.11 (br m, 2H), 3.45 (br s, 1H), 3.30 (m, 2H), 3.17 (d, J = 9.2 Hz, 1H), 2.78 (br q, J = 7.2 Hz, 3H), 2.28 (s, 3H), 1.87 (br d, J = 8.0 Hz, 1H), 1.75 (br d, J = 9.2 Hz, 1H), 1.21 (t, J = 7.6 Hz, 3H) in pyridine-d₅. | 500.4 |
| 72 | 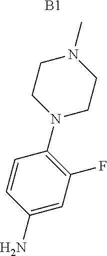 | 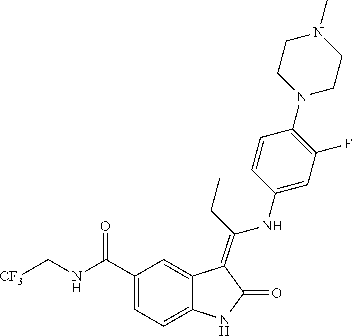 [SMILES: CC/C(Nc1ccc(N2CCN(C)CC2)c(F)c1)=C1/C(=O)Nc2ccc(C(=O)NCC(F)(F)F)cc21] | K | 36 | 12.03 (s, 1H), 10.89 (s, 1H), 8.90 (br t, J = 6.0 Hz, 1H), 7.80 (s, 1H), 7.63 (d, J = 8.8 Hz, 1H), 7.20 (d, J = 13.2 Hz, 1H) 7.09 (m, 2H), 6.95 (d, J = 8.0 Hz, 1H), 4.03-4.12 (m, 2H), 3.05 (br m, 4H), 2.83 (br q, J = 7.6 Hz, 2H), 2.23 (s, 3H), 1.23 (t, J = 7.6 Hz, 3H), 4Hs are under solvent peaks. | 506.4 |
| 73 | 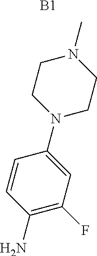 | 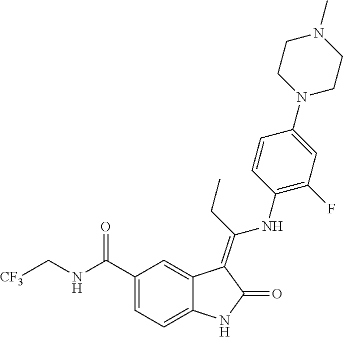 [SMILES: CC/C(Nc1ccc(N2CCN(C)CC2)cc1F)=C1/C(=O)Nc2ccc(C(=O)NCC(F)(F)F)cc21] | K | 25 | 11.66 (s, 1H), 10.89 (s, 1H), 7.79 (s, 1H), 7.61 (br d, J = 8.4 Hz, 1H), 7.28 (t, J = 9.2 Hz, 1H), 6.92-6.97 (m, 2H), 6.83 (dd, J = 6.8, 2.0 Hz, 1H), 4.07 (m, 2H), 3.22 (br m, 4H), 2.73 (br q, J = 7.6 Hz, 2H), 2.44 (br m, 4H), 2.22 (s, 3H), 1.16 (t, J = 7.6 Hz, 3H), one NH is under solvent peaks. | 506.5 |
| 74 | 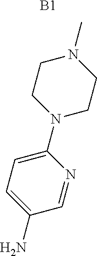 | 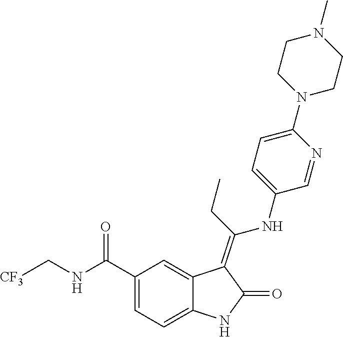 [SMILES: CC/C(Nc1ccc(N2CCN(C)CC2)nc1)=C1/C(=O)Nc2ccc(C(=O)NCC(F)(F)F)cc21] | K | 56 | 11.82 (s, 1H), 10.88 (s, 1H), 8.90 (t, J = 6.3 Hz, 1H), 8.11 (d, J = 2.7 Hz, 1H), 7.80 (d, J = 1.6 Hz, 1H), 7.62 (dd, J = 8.3, 1.5 Hz, 1H), 7.56 (dd, J = 9.0, 2.8 Hz, 1H), 6.96 (d, J = 8.1 Hz, 1H), 6.92 (d, J = 9.0 Hz, 1H), 4.08 (m, 2H), 3.47-3.59 (m, 4H), 2.75 (q, J = 7.5 Hz, 2H), 2.41 (m, 4H), 2.23 (s, 3H), 1.19 (t, J = 7.5 Hz, 3H). | 489.2 |
| 75 | 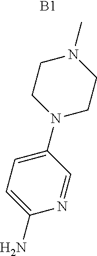 | 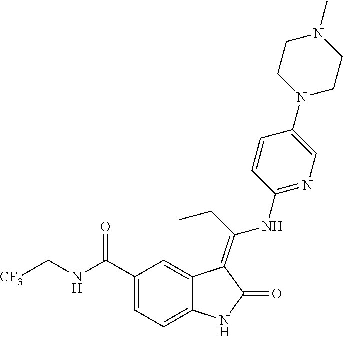 [SMILES: CC/C(Nc1ccc(N2CCN(C)CC2)cn1)=C1/C(=O)Nc2ccc(C(=O)NCC(F)(F)F)cc21] | K | 28 | 12.58 (s, 1H), 10.95 (s, 1H), 8.92 (t, J = 6.3 Hz, 1H), 8.13 (d, J = 3.1 Hz, 1H), 7.89 (d, J = 1.7 Hz, 1H), 7.64 (dd, J = 8.1, 1.6 Hz, 1H), 7.47 (dd, J = 9.0, 3.1 Hz, 1H), 7.08 (d, J = 8.8 Hz, 1H), 6.97 (d, J = 8.2 Hz, 1H), 4.02-4.12 (m, 2H), 3.27 (q, J = 7.4 Hz, 2H), 3.18-3.23 (m, 4H), 2.44-2.48 (m, 4H), 2.23 (s, 3H), 1.38 (t, J = 7.4 Hz, 3H). | 489.2 |
| 76 | 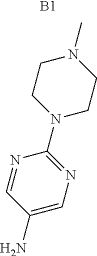 | 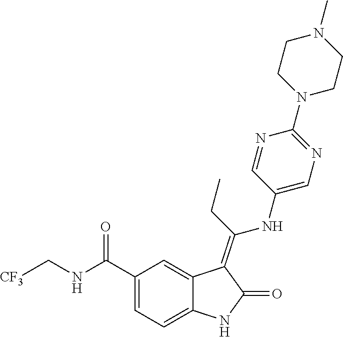 [SMILES: CC/C(Nc1cnc(N2CCN(C)CC2)nc1)=C1/C(=O)Nc2ccc(C(=O)NCC(F)(F)F)cc21] | K | 53 | 11.64 (s, 1H), 10.90 (s, 1H), 8.92 (t, J = 6.3 Hz, 1H), 8.42 (s, 2H), 7.80 (d, J = 1.7 Hz, 1H), 7.63 (dd, J = 8.2, 1.6 Hz, 1H), 6.96 (d, J = 8.2 Hz, 1H), 4.08 (m, 2H), 3.76 (dd, J = 6.1, 4.0 Hz, 4H), 2.73 (q, J = 7.5 Hz, 2H), 2.38 (t, J = 5.1 Hz, 4H), 2.23 (s, 3H), 1.18 (t, J = 7.6 Hz, 3H). | 490.2 |
| 77 | 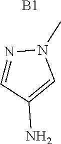 | 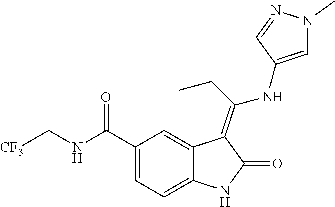 [SMILES: CC/C(Nc1cnn(C)c1)=C1/C(=O)Nc2ccc(C(=O)NCC(F)(F)F)cc21] | K | 45 | 11.70 (s, 1H), 10.86 (s, 1H), 8.90 (t, J = 6.3 Hz, 1H), 7.94 (s, 1H), 7.80 (d, J = 1.6 Hz, 1H), 7.61 (dd, J = 8.1, 1.6 Hz, 1H), 7.55 (s, 1H), 6.95 (d, J = 8.1 Hz, 1H), 4.08 (m, 2H), 3.87 (s, 3H), 2.82 (q, J = 7.5 Hz, 2H), 1.24 (t, J = 7.6 Hz, 3H). | 394.2 |
| 78 | 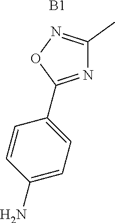 | 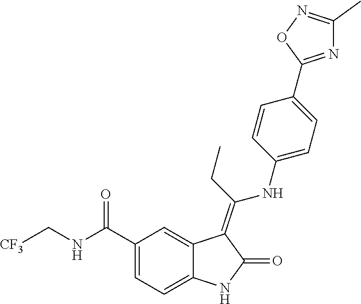 [SMILES: CC/C(Nc1ccc(-c2nc(C)no2)cc1)=C1/C(=O)Nc2ccc(C(=O)NCC(F)(F)F)cc21] | K | 4 | 12.46 (s, 1H), 11.04 (s, 1H), 8.95 (t, J = 5.6 Hz, 1H), 8.15 (d, J = 8.8 Hz, 2H), 7.87 (s, 1H), 7.67 (d, J = 8.0 Hz, 1H), 7.53 (d, J = 8.8 Hz, 2H), 6.99 (d, J = 8.0 Hz, 1H), 4.09 (m, 2H), 3.02 (q, J = 7.2 Hz, 2H), 2.42 (s, 3H), 1.34 (t, J = 7.6 Hz, 3H). | 472.3 |
| 79 | 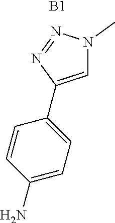 | 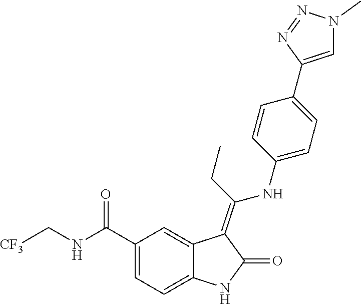 [SMILES: CC/C(Nc1ccc(-c2cn(C)nn2)cc1)=C1/C(=O)Nc2ccc(C(=O)NCC(F)(F)F)cc21] | K | 6 | 12.25 (br s, 1H), 10.94 (s, 1H), 8.91 (t, J = 6.4 Hz, 1H), 8.56 (s, 1H), 7.93 (d, J = 8.4 Hz, 2H), 7.84 (s, 1H), 7.64 (d, J = 8.0 Hz, 1H), 7.41 (d, J = 8.4 Hz, 2H), 6.99 (d, J = 8.0 Hz, 1H), 4.11 (s, 3H), 4.06 (m, 2H), 2.91 (q, J = 7.2 Hz, 2H), 1.29 (t, J = 7.6 Hz, 3H). | 471.2 |
| 80 | 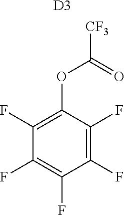 | 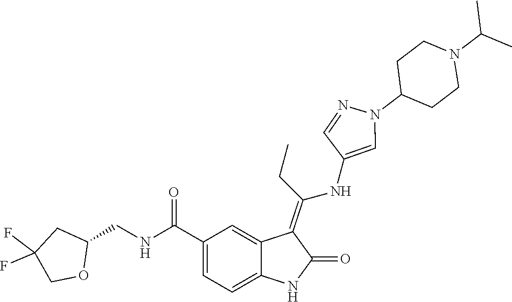 [SMILES: CC/C(Nc1cnn(C2CCN(C(C)C)CC2)c1)=C1/C(=O)Nc2ccc(C(=O)NC[C@H]3CC(F)(F)CO3)cc21] | L | 43 | 11.71 (s, 1H), 10.81 (s, 1H), 8.50 (t, J = 5.9 Hz, 1H), 8.02 (s, 1H), 7.77 (d, J = 1.7 Hz, 1H), 7.54-7.58 (m, 2H), 6.92 (d, J = 8.2 Hz, 1H), 4.31 (m, 1H), 4.00-4.17 (m, 2H), 3.85 (m, 1H), 3.50 (dt, J = 13.7, 6.0 Hz, 1H), 3.42 (dt, J = 13.7, 5.4 Hz, 1H), 2.85-2.92 (m, 2H), 2.81 (m, 2H), 2.73 (m, 1H), 2.55 (m, 1H), 2.25 (m, 3H), 2.02 (m, 2H), 1.90 (m, 2H), 1.24 (t, J = 7.6 Hz, 3H), 0.98 (d, J = 6.6 Hz, 6H). | 543.4 |
| | 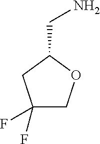 | | | | | |
| 81 | 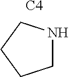 | 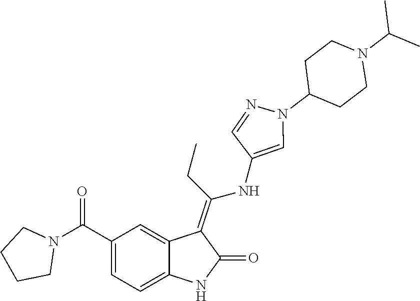 [SMILES: CC/C(Nc1cnn(C2CCN(C(C)C)CC2)c1)=C1/C(=O)Nc2ccc(C(=O)N3CCCC3)cc21] | I | 54 | 11.67 (s, 1H), 10.72 (s, 1H), 8.01 (s, 1H), 7.54 (s, 1H), 7.39 (s, 1H), 7.19 (d, J = 8.1 Hz, 1H), 6.90 (d, J = 8.0 Hz, 1H), 4.09 (m, 1H), 3.45 (m, 4H), 2.86 (d, J = 11.1 Hz, 2H), 2.72 (q, J = 7.6 Hz, 3H), 2.24 (t, J = 11.5 Hz, 2H), 2.00 (m, 2H), 1.79-1.92 (m, 6H), 1.22 (t, J = 7.5 Hz, 3H), 0.97 (d, J = 6.6 Hz, 6H). | 477.4 |
| 82 | 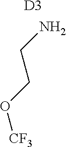 | 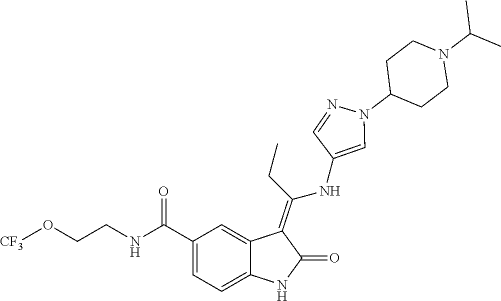 [SMILES: CC/C(Nc1cnn(C2CCN(C(C)C)CC2)c1)=C1/C(=O)Nc2ccc(C(=O)NCCOC(F)(F)F)cc21] | L (T3P) | 23 | 11.72 (s, 1H), 10.81 (s, 1H), 8.57 (t, J = 5.6 Hz, 1H), 8.03 (s, 1H), 7.77 (d, J = 1.6 Hz, 1H), 7.53-7.62 (m, 2H), 6.93 (d, J = 8.1 Hz, 1H), 4.19 (t, J = 5.5 Hz, 2H), 4.11 (br s, 1H), 3.56 (q, J = 5.5 Hz, 2H), 2.88 (br s, 2H), 2.81 (q, J = 7.6 Hz, 2H), 2.74 (br s, 1H), 2.26 (br s, 2H), 1.82- 2.10 (m, 4H), 1.24 (t, J = 7.6 Hz, 3H), 0.90- 1.08 (br s, 6H). | 535.2 |
| 83 | 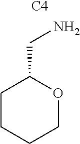 | 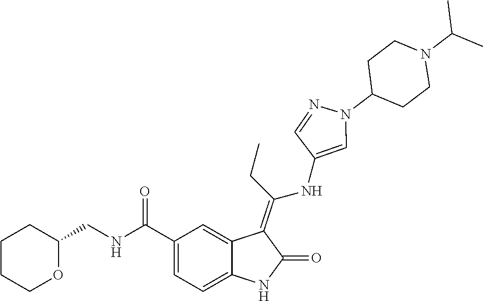 [SMILES: CC/C(Nc1cnn(C2CCN(C(C)C)CC2)c1)=C1/C(=O)Nc2ccc(C(=O)NC[C@H]3CCCCO3)cc21] | I | 39 | 11.70 (s, 1H), 10.78 (s, 1H), 8.34 (t, J = 5.8 Hz, 1H), 8.03 (s, 1H), 7.77 (s, 1H), 7.53-7.60 (m, 2H), 6.91 (d, J = 8.1 Hz, 1H), 4.11 (br s, 1H), 3.86 (dd, J = 10.8, 3.2 Hz, 1H), 3.41 (m, 1H), 3.28-3.35 (m, 2H), 3.20-3.28 (m, 2H), 2.88 (br s, 1H), 2.76-2.84 (m, 3H), 2.26 (br m, 2H), 2.03 (br m, 2H), 1.92 (br m, 2H), 1.77 (m, 1H), 1.60 (m, 1H), 1.40-1.49 (m, 3H), 1.23 (t, J = 7.6 Hz, 3H), 1.15 (m, 1H), 1.00 (br s, 6H). | 521.4 |
| 84 | 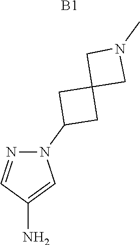 | 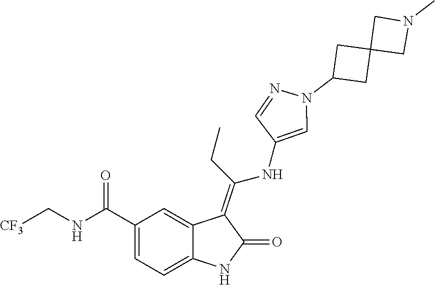 [SMILES: CC/C(Nc1cnn(C2CC3(C2)CN(C)C3)c1)=C1/C(=O)Nc2ccc(C(=O)NCC(F)(F)F)cc21] | K | 53 | 11.72 (s, 1H), 10.86 (s, 1H), 8.90 (t, J = 6.3 Hz, 1H), 8.02 (s, 1H), 7.80 (s, 1H), 7.58-7.63 (m, 2H), 6.95 (d, J = 8.2 Hz, 1H), 4.73 (m, 1H), 4.04-4.11 (m, 2H), 3.22 (s, 2H), 3.13 (s, 2H), 2.81 (q, J = 7.6 Hz, 2H), 2.56 (dt, J = 8.5, 2.3 Hz, 4H), 2.19 (s, 3H), 1.24 (t, J = 7.6 Hz, 3H). | 489.2 |
| 85 | 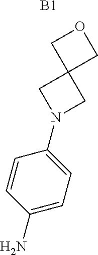 | 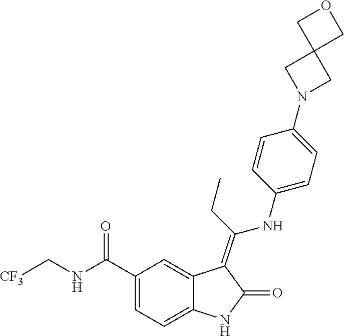 [SMILES: CC/C(Nc1ccc(N2CC3(COC3)C2)cc1)=C1/C(=O)Nc2ccc(C(=O)NCC(F)(F)F)cc21] | K | 56 | 11.93 (s, 1H), 10.84 (s, 1H), 8.88 (br t, J = 6.4 Hz, 1H), 7.77 (s, 1H), 7.58 (d, J = 8.0 Hz, 1H), 7.13 (d, J = 8.4 Hz, 2H), 6.94 (d, J = 8.0 Hz, 1H), 6.49 (d, J = 8.4 Hz, 2H), 4.72 (s, 4H), 4.06 (q, J = 8.0 Hz, 2H), 4.02 (s, 4H), 3.39 (m, 1H), 2.72- 2.79 (m, 2H), 1.19 (t, J = 7.6 Hz, 3H). | 487.4 |
| 86 | 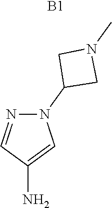 | 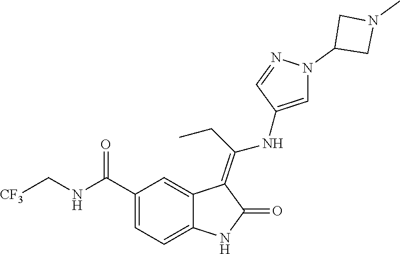 [SMILES: CC/C(Nc1cnn(C2CN(C)C2)c1)=C1/C(=O)Nc2ccc(C(=O)NCC(F)(F)F)cc21] | K | 60 | 11.74 (s, 1H), 10.87 (s, 1H), 8.90 (t, J = 6.3 Hz, 1H), 8.12 (s, 1H), 7.81 (d, J = 1.6 Hz, 1H), 7.65 (s, 1H), 7.61 (dd, J = 8.2, 1.6 Hz, 1H), 6.96 (d, J = 8.2 Hz, 1H), 4.95 (m, 1H), 4.04-4.11 (m, 2H), 3.72-3.69 (m, 2H), 3.38 (m, 2H), 2.82 (q, J = 7.5 Hz, 2H), 2.32 (s, 3H), 1.25 (t, J = 7.6 Hz, 3H). | 449.2 |
| 87 | 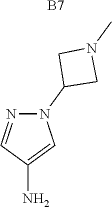 | 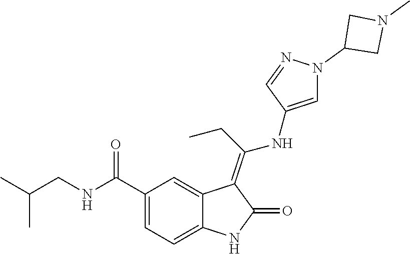 [SMILES: CC/C(Nc1cnn(C2CN(C)C2)c1)=C1/C(=O)Nc2ccc(C(=O)NCC(C)C)cc21] | K | 14 | 11.72 (s, 1H), 10.78 (s, 1H), 8.30 (t, J = 5.9 Hz, 1H), 8.11 (s, 1H), 7.76 (d, J = 1.6 Hz, 1H), 7.65 (s, 1H), 7.55 (dd, J = 8.2, 1.6 Hz, 1H), 6.91 (d, J = 8.1 Hz, 1H), 4.96 (m, 1H), 3.70-3.76 (m, 2H), 3.38-3.45 (m, 2H), 3.07 (t, J = 6.4 Hz, 2H), 2.82 (q, J = 7.5 Hz, 2H), 2.34 (s, 3H), 1.84 (m, 1H), 1.24 (t, J = 7.5 Hz, 3H), 0.89 (d, J = 6.7 Hz, 6H). | 423.2 |
| 88 | 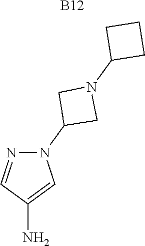 | 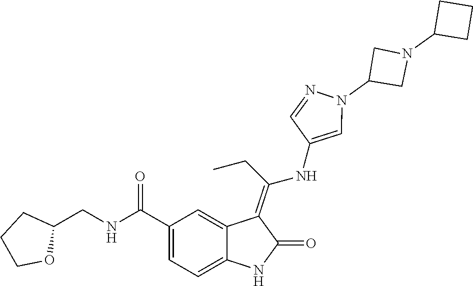 [SMILES: CC/C(Nc1cnn(C2CN(C3CCC3)C2)c1)=C1/C(=O)Nc2ccc(C(=O)NC[C@H]3CCCO3)cc21] | K | 24 | 11.73 (s, 1H), 10.79 (s, 1H), 8.37 (t, J = 5.9 Hz, 1H), 8.10 (s, 1H), 7.77 (d, J = 1.7 Hz, 1H), 7.66 (s, 1H), 7.56 (dd, J = 8.1, 1.6 Hz, 1H), 6.91 (d, J = 8.1 Hz, 1H), 4.98 (m, 1H), 3.98 (m, 1H), 3.77 (m, 1H), 3.58-3.70 (m, 3H), 3.48 (br s, 2H), 2.74- 2.90 (m, 2H), 1.75- 2.00 (m, 8H), 1.56- 1.74 (m, 3H), 1.24 (t, J = 7.6 Hz, 3H). | 491.2 |
| 89 | 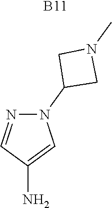 | 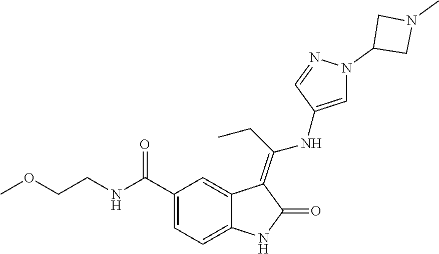 | K | 48 | 11.73 (s, 1H), 10.80 (s, 1H), 8.37 (t, J = 5.4 Hz, 1H), 8.11 (s, 1H), 7.77 (d, J = 1.6 Hz, 1H), 7.66 (s, 1H), 7.56 (dd, J = 8.2, 1.6 Hz, 1H), 6.91 (d, J = 8.1 Hz, 1H), 4.97 (m, 1H), 3.72-3.76 (m, 2H), 3.43 (m, 6H), 3.27 (s, 3H), 2.82 (q, J = 7.5 Hz, 2H), 2.35 (s, 3H), 1.24 (t, J = 7.6 Hz, 3H). | 425.2 |
| 90 | 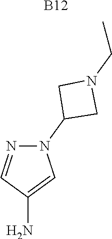 | 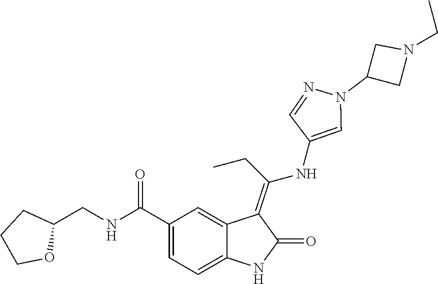 [SMILES: CC/C(Nc1cnn(C2CN(CC)C2)c1)=C1/C(=O)Nc2ccc(C(=O)NC[C@H]3CCCO3)cc21] | K | 19 | 11.73 (s, 1H), 10.79 (s, 1H), 8.37 (t, J = 5.9 Hz, 1H), 8.11 (s, 1H), 7.77 (d, J = 1.6 Hz, 1H), 7.65 (s, 1H), 7.56 (dd, J = 8.1, 1.5 Hz, 1H), 6.91 (d, J = 8.2 Hz, 1H), 4.98 (m, 1H), 3.98 (m, 1H), 3.78 (m, 1H), 3.70 (t, J = 7.7 Hz, 2H), 3.67 (m, 1H), 3.34- 3.40 (m, 2H), 2.76- 2.88 (m, 2H), 1.75- 1.93 (m, 3H), 1.60 (m, 1H), 1.24 (t, J = 7.6 Hz, 3H), 0.92 (t, J = 7.2 Hz, 3H). | 465.2 |
| 91 | 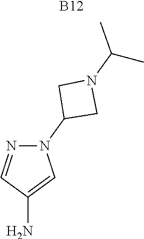 | 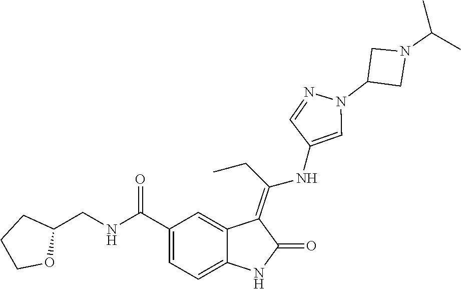 [SMILES: CC/C(Nc1cnn(C2CN(C(C)C)C2)c1)=C1/C(=O)Nc2ccc(C(=O)NC[C@H]3CCCO3)cc21] | K | 37 | 11.73 (s, 1H), 10.79 (s, 1H), 8.37 (t, J = 5.9 Hz, 1H), 8.11 (s, 1H), 7.77 (d, J = 1.6 Hz, 1H), 7.64 (s, 1H), 7.56 (dd, J = 8.2, 1.5 Hz, 1H), 6.91 (d, J = 8.2 Hz, 1H), 4.91 (m, 1H), 3.98 (m, 1H), 3.78 (m, 1H), 3.60-3.68 (m, 3H), 2.77-2.87 (m, 2H), 2.41 (m, 1H), 1.76- 1.93 (m, 3H), 1.59 (m, 1H), 1.24 (t, J = 7.6 Hz, 3H), 0.90 (d, J = 6.1 Hz, 6H). | 479.2 |
| 92 | 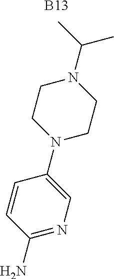 | 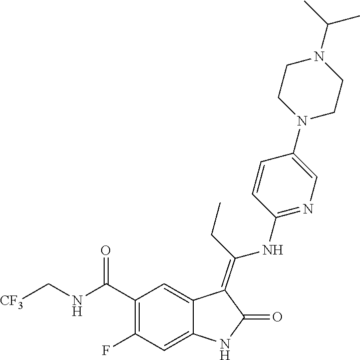 [SMILES: CC/C(Nc1ccc(N2CCN(C(C)C)CC2)cn1)=C1/C(=O)Nc2cc(F)c(C(=O)NCC(F)(F)F)cc21] | K | 10 | 12.40 (s, 1H), 11.05 (s, 1H), 8.63 (q, J = 6.0, 1H), 8.12 (d, J = 2.8 Hz, 1H ), 7.60 (d, J = 6.8 Hz, 1H), 7.45 (dd, J = 8.8, 2.8 Hz, 1H), 7.07 (d, J = 8.8 Hz, 1H), 6.78 (d, J = 10.8 Hz, 1H), 4.08 (m, 2H), 3.16-3.21 (m, 6H), 2.68 (m, 1H ), 2.56 (m, 4H), 1.37 (t, J = 7.6 Hz, 3H), 1.00 (d, J = 6.4 Hz, 6H). | 535.5 |
| 93 | 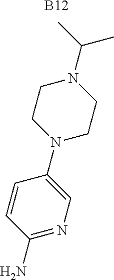 | 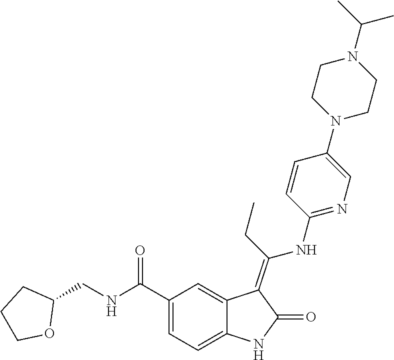 [SMILES: CC/C(Nc1ccc(N2CCN(C(C)C)CC2)cn1)=C1/C(=O)Nc2ccc(C(=O)NC[C@H]3CCCO3)cc21] | K | 25 | 12.56 (s, 1H), 10.87 (s, 1H), 8.39 (br t, J = 6.0 Hz, 1H), 8.11 (d, J = 2.8 Hz, 1H), 7.84 (s, 1H), 7.59 (d, J = 8.4 Hz, 1H), 7.46 (dd, J = 8.8, 2.4 Hz, 1H), 7.07 (d, J = 9.2 Hz, 1H), 6.93 (d, J = 8.0 Hz, 1H), 3.98 (m, 1H), 3.77 (m, 1H), 3.61 (m, 1H), 3.26-3.32 (br m, 4H), 3.17 (br m, 4H), 2.70 (m, 1H), 2.58 (br m, 4H), 1.76-1.92 (m, 3H), 1.60 (m, 1H), 1.37 (t, J = 7.2 Hz, 3H), 1.08 (d, J = 6.4 Hz, 6H). | 519.3 |
| 94 | 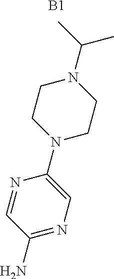 | 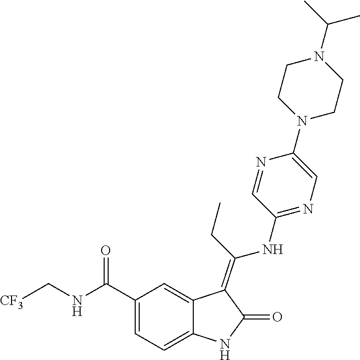 [SMILES: CC/C(Nc1cnc(N2CCN(C(C)C)CC2)cn1)=C1/C(=O)Nc2ccc(C(=O)NCC(F)(F)F)cc21] | K | 10 | 12.44 (s, 1H), 10.96 (s, 1H), 8.92 (t, J = 6.4 Hz, 1H), 8.19 (s, 1H), 8.12 (s, 1H), 7.86 (s, 1H), 7.63 (dd, J = 8.0, 1.2 Hz, 1H), 6.97 (d, J = 8.0 Hz, 1H), 6.99 (d, J = 8.0 Hz, 1H), 4.07 (m, 2H), 3.53 (br s, 4H), 3.10 (q, J = 7.2 Hz, 2H), 2.71 (m, 1H), 2.55 (m, 4H), 1.30 (t, J = 7.2 Hz, 3H), 1.01 (d, J = 6.4 Hz, 6H). | 518.4 |
| 95 | 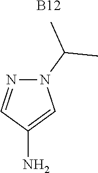 | 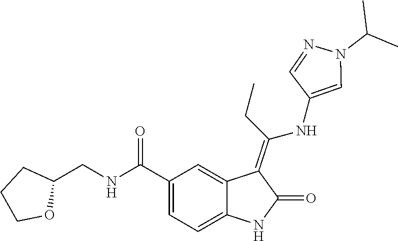 [SMILES: CC/C(Nc1cnn(C(C)C)c1)=C1/C(=O)Nc2ccc(C(=O)NC[C@H]3CCCO3)cc21] | K | 33 | 11.71 (s, 1H), 10.77 (s, 1H), 8.35 (t, J = 6.4 Hz, 1H), 7.99 (s, 1H), 7.76 (s, 1H), 7.55 (m, 2H), 6.91 (d, J = 8.0 Hz, 1H), 4.50 (m, 1H), 3.99 (m, 1H), 3.78 (m, 1H), 3.65 (m, 1H), 2.77- 2.86 (m, 2H), 1.75- 1.93 (m, 3H), 1.59 (m, 1H) 1.44 (d, J = 6.4 Hz, 6H), 1.24 (t, J = 7.2 Hz, 3H). | 424.2 |
| 96 | 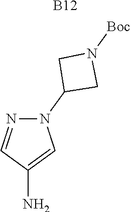 | 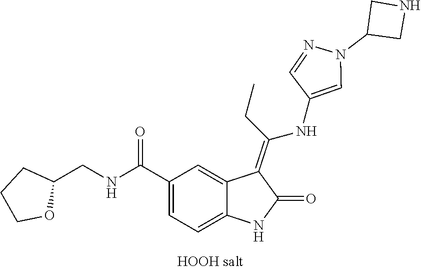 [SMILES: CC/C(Nc1cnn(C2CNC2)c1)=C1/C(=O)Nc2ccc(C(=O)NC[C@H]3CCCO3)cc21] | K & HCOOH | 18 | 11.74 (s, 1H), 10.81 (s, 1H), 8.38 (t, J = 5.9 Hz, 1H), 8.31 (s, 1H), 8.13 (s, 1H), 7.77 (d, J = 1.6 Hz, 1H), 7.70 (s, 1H), 7.56 (dd, J = 8.2, 1.6 Hz, 1H), 6.92 (d, J = 8.1 Hz, 1H), 5.26 (m, 1H), 4.06 (t, J = 8.0 Hz, 2H), 3.89-4.02 (m, 3H), 3.77 (m, 1H), 3.62 (m, 1H), 3.25-3.37 (m, 2H), 2.77-2.88 (m, 2H), 1.73-1.95 (m, 4H), 1.60 (m, 1H), 1.25 (t, J = 7.5 Hz, 3H). | 437.2 |
| 97 | 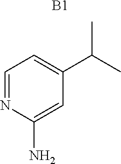 | 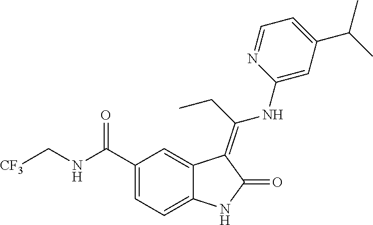 [SMILES: CC/C(Nc1cc(C(C)C)ccn1)=C1/C(=O)Nc2ccc(C(=O)NCC(F)(F)F)cc21] | K | 11 | 12.70 (s, 1H), 11.05 (s, 1H), 8.95 (t, J = 6.3 Hz, 1H), 8.31 (d, J = 5.2 Hz, 1H), 7.93 (s, 1H), 7.67 (d, J = 8.1 Hz, 1H), 7.08 (d, J = 5.2 Hz, 1H), 6.96-7.00 (m, 2H), 4.08 (m, 2H), 3.44 (q, J = 7.3 Hz, 2H), 2.94 (m, 1H), 1.42 (t, J = 7.3 Hz, 3H), 1.23 (d, J = 6.9 Hz, 6H). | 433.2 |
| 98 | 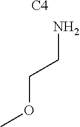 | 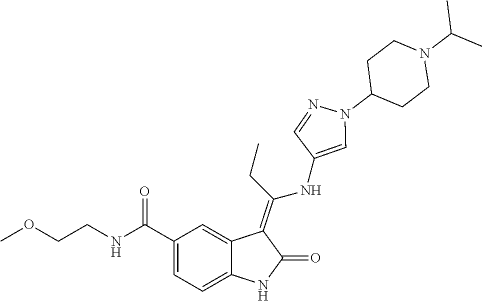 [SMILES: CC/C(Nc1cnn(C2CCN(C(C)C)CC2)c1)=C1/C(=O)Nc2ccc(C(=O)NCCOC)cc21] | I | 35 | 11.70 (s, 1H), 10.77 (s, 1H), 8.35 (t, J = 5.4 Hz, 1H), 8.01 (s, 1H), 7.76 (s, 1H), 7.55 (m, 2H), 6.90 (d, J = 8.1 Hz, 1H), 4.10 (br m, 1H), 3.38-3.45 (m, 4H), 3.26 (s, 3H), 2.87 (br m, 2H), 2.78 (q, J = 7.2 Hz, 2H), 2.74 (br s, 1H), 2.25 (br m, 2H), 2.01 (m, 2H), 1.90 (m, 2H), 1.23 (t, J = 7.5 Hz, 3H), 0.98 (d, J = 6.5 Hz, 6H). | 481.3 |
| 99 | 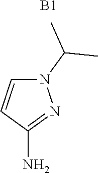 | 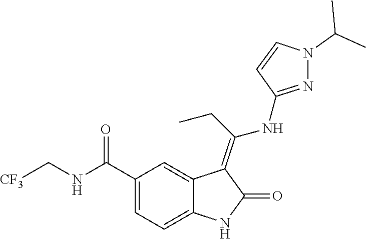 [SMILES: CC/C(Nc1ccn(C(C)C)n1)=C1/C(=O)Nc2ccc(C(=O)NCC(F)(F)F)cc21] | K | 9 | 12.31 (s, 1H), 10.93 (s, 1H), 8.92 (t, J = 6.3 Hz, 1H), 7.86 (d, J = 1.6 Hz, 1H), 7.82 (d, J = 2.3 Hz, 1H), 7.64 (dd, J = 8.1, 1.6 Hz, 1H), 6.97 (d, J = 8.2 Hz, 1H), 6.21 (d, J = 2.3 Hz, 1H), 4.47 (m, 1H), 4.09 (m, 2H), 3.08 (q, J = 7.5 Hz, 2H), 1.44 (d, J = 6.6 Hz, 6H), 1.35 (t, J = 7.5 Hz, 3H). | 422.2 |
| 100 | 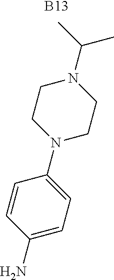 | 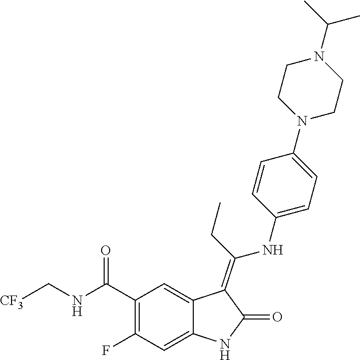 [SMILES: CC/C(Nc1ccc(N2CCN(C(C)C)CC2)cc1)=C1/C(=O)Nc2cc(F)c(C(=O)NCC(F)(F)F)cc21] | K | 36 | 11.82 (s, 1H), 10.94 (s, 1H), 8.58 (dd, J =10.4, 6.4 Hz, 1H), 7.52 (d, J = 6.8 Hz, 1H), 7.16 (d, J = 8.8 Hz, 2H), 6.99 (d, J = 9.2 Hz, 2H), 6.77 (d, J = 11.2 Hz, 1H), 4.02-4.11 (m, 2H), 3.16 (br m, 4H), 2.69 (m, 2H), 2.58 (br m, 4H), 1.20 (t, J = 7.6 Hz, 3H), 1.03 (d, J = 6.4 Hz, 6H), one NH is under solvent peaks. | 534.2 |
| 101 | 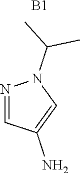 | 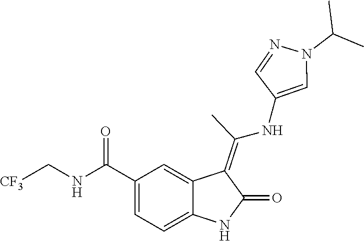 [SMILES: C/C(Nc1cnn(C(C)C)c1)=C1/C(=O)Nc2ccc(C(=O)NCC(F)(F)F)cc21] | K | 21 | 11.85 (s, 1H), 10.82 (s, 1H), 8.92 ( t, J = 6.4 Hz, 1H), 7.99 (s, 1H), 7.93 ( d, J = 6.0 Hz, 1H), 7.61 (dd, J = 8.0, 1.6 Hz, 1H),7.56 (s, 1H), 6.93 (d, J = 8.0 Hz, 1H), 4.49 (m, 1H), 4.03-4.12 (m, 2H), 2.54 (s, 3H), 1.14 (d, J = 6.8 Hz, 6H). | 408.3 |
| 102 | 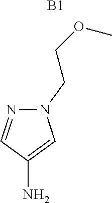 | 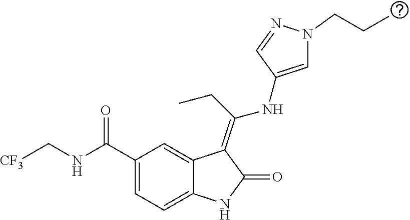 [SMILES: CCCn1cc(N/C(CC)=C2\C(=O)Nc3ccc(C(=O)NCC(F)(F)F)cc32)cn1] | K | 10 | 11.71 (s, 1H), 10.86 (s, 1H), 8.89 (t, J = 6.0 Hz, 1H), 7.95 (s, 1H), 7.80 (s, 1H), 7.61 (dd, J = 8.4, 1.6 Hz, 1H), 7.58 (s, 1H), 6.96 (d, J = 8.4 Hz, 1H), 4.25 (t, J = 5.6 Hz, 2H), 4.03-4.12 (m, 2H), 3.70 (t, J = 5.6 Hz, 2H), 3.24 (s, 3H), 2.82 (q, J = 7.6 Hz, 2H), 1.23 (t, J = 7.6 Hz, 3H). | 438.2 |
| 103 | 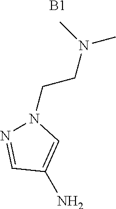 | 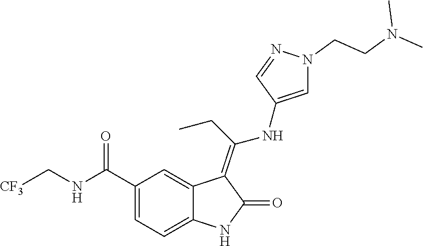 [SMILES: CC/C(Nc1cnn(CCN(C)C)c1)=C1/C(=O)Nc2ccc(C(=O)NCC(F)(F)F)cc21] | K | 10 | 11.71 (s, 1H), 10.85 (s, 1H), 8.88 (t, J = 6.0 Hz, 1H), 7.98 (s, 1H), 7.80 (s, 1H), 7.6 (d, J = 8.0 Hz, 1H), 7.55 (s, 1H), 6.96 (d, J = 8.0 Hz, 1H), 4.20 (t, J = 6.4 Hz, 2H), 4.03-4.09 (m, 2H), 2.81 (q, J = 7.2 Hz, 2H), 2.68 (m, 2H), 2.17 (s, 6H), 1.23 (t, J = 7.6 Hz, 3H). | 451.3 |
| 104 | 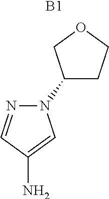 | 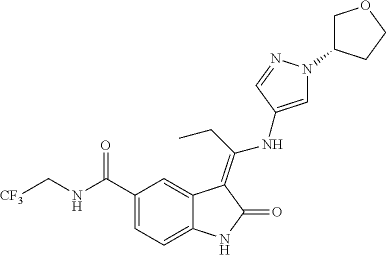 [SMILES: CC/C(Nc1cnn([C@H]2CCOC2)c1)=C1/C(=O)Nc2ccc(C(=O)NCC(F)(F)F)cc21] | K | 14 | 11.73 (s, 1H), 10.87 (s, 1H), 8.90 (t, J = 6.4 Hz, 1H), 8.03 (s, 1H), 7.80 (s, 1H), 7.61 (s, 1H), 7.60 (dd, J = 7.0, 1.2 Hz, 1H), 6.94 (d, J = 8.4 Hz, 1H), 5.03 (m, 1H), 4.03-4.12 (m, 2H), 3.95-4.00 (m, 2H), 3.92 (m, 1H), 3.82(m, 1H), 2.83 (q, J = 7.6 Hz, 2H), 2.27- 2.33 (m, 2H), 1.24 (t, J = 7.2 Hz, 3H). | 450.3 |
| 105 | 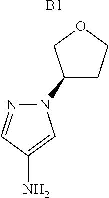 | 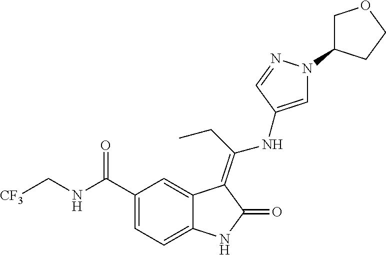 [SMILES: CC/C(Nc1cnn([C@@H]2CCOC2)c1)=C1/C(=O)Nc2ccc(C(=O)NCC(F)(F)F)cc21] | K | 18 | 11.73 (s, 1H), 10.87 (s, 1H), 8.90 (t, J = 6.4 Hz, 1H), 8.03 (s, 1H), 7.80 (s, 1H), 7.61 (s, 1H), 7.60 (dd, J = 7.0, 1.2 Hz, 1H), 6.94 (d, J = 8.4 Hz, 1H), 5.03 (m, 1H), 4.03-4.12 (m, 2H), 3.95-4.00 (m, 2H), 3.92 (m, 1H), 3.82 (m, 1H), 2.83 (q, J = 7.6 Hz, 2H), 2.27-2.33 (m, 2H), 1.24 (t, J = 7.2 Hz, 3H). | 450.3 |
| 106 | 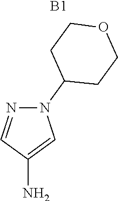 | 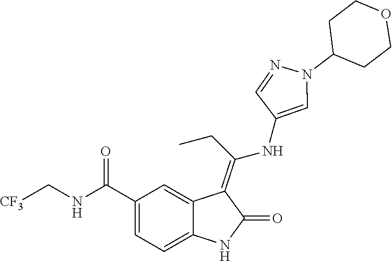 [SMILES: CC/C(Nc1cnn(C2CCOCC2)c1)=C1/C(=O)Nc2ccc(C(=O)NCC(F)(F)F)cc21] | K | 12 | 11.73 (s, 1H), 10.87 (s, 1H),8.90 (t, J = 6.0 Hz, 1H), 8.06 (s, 1H),7.80 (s, 1H), 7.59-7.63 (m, 2H), 6.94 (d, J = 8.4 Hz, 1H), 4.42 (m, 1H), 4.03-4.12 (m, 2H), 3.94-3.99 (m, 2H), 3.43-3.50 (m, 2H), 2.82 (q, J = 7.6 Hz, 2H), 1.90-2.00 (m, 4H), 1.24 (t, J = 7.6 Hz, 3H). | 464.2 |
| 107 | 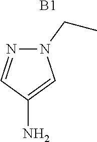 | 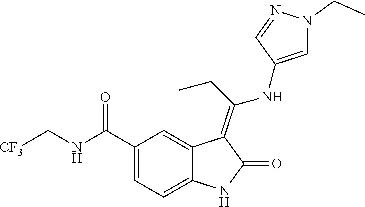 [SMILES: CC/C(Nc1cnn(CC)c1)=C1/C(=O)Nc2ccc(C(=O)NCC(F)(F)F)cc21] | K | 38 | 11.72 (s, 1H), 10.84 (s, 1H), 8.88 (t, J = 6.4 Hz, 1H), 7.98 (s, 1H), 7.80 (s, 1H), 7.59 (d, J = 8.0 Hz, 1H), 7.56 (s, 1H), 6.94 (d, J = 8.0 Hz, 1H), 4.14 (q, J = 7.2 Hz, 2H), 4.03-4.09 (m, 2H), 2.83 (q, J = 7.2 Hz, 2H), 1.39 (t , J = 7.2 Hz, 3H), 1.24 (t, J = 7.6 Hz, 3H). | 408.3 |
| 108 | 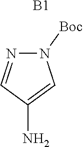 | 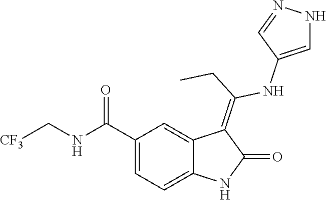 [SMILES: CC/C(Nc1cn[nH]c1)=C1/C(=O)Nc2ccc(C(=O)NCC(F)(F)F)cc21] | K | 48 | 13.05 (s, 1H), 11.70 (s, 1H), 10.84 (s, 1H), 8.89 (br m, 1H), 7.97 (br s, 1H), 7.80 (br s, 1H), 7.61 (br m, 2H), 6.94 (br d, J = 7.2 Hz, 1H), 4.07 (br m, 2H), 2.81 (br m, 2H), 1.22 (br m, 3H). | 380.2 |
| 109 | 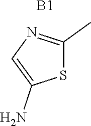 | 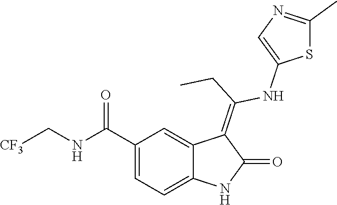 [SMILES: CC/C(Nc1cnc(C)s1)=C1/C(=O)Nc2ccc(C(=O)NCC(F)(F)F)cc21] | K | 8 | 7.91 (s, 1H), 7.63 (d, J = 7.6 Hz, 1H), 7.53 ( s, 1H), 7.04 (d, J = 7.6 Hz, 1H), 4.06-4.13 (m, 2H), 2.93 (d, J = 7.6 Hz, 2H), 2.70 (s, 3H), 1.35 (t, J = 7.2 Hz, 3H) in methanol-d₄, 3NHs are under solvent peaks. | 450.2 |
| 110 | 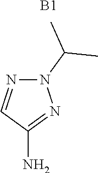 | 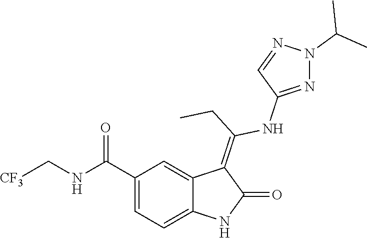 [SMILES: CC/C(Nc1cnn(C(C)C)n1)=C1/C(=O)Nc2ccc(C(=O)NCC(F)(F)F)cc21] | K | 8 | 12.34 (s, 1H), 11.01 (s, 1H), 8.93 (t, J = 6.0 Hz, 1H), 7.87 (s, 1H), 7.81 (s, 1H), 7.65 (d, J = 8.4 Hz, 1H), 6.97 (d, J = 8.0 Hz, 1H), 4.78 (m, 1H), 4.09 (m, 2H), 3.03 (q, J = 7.6 Hz, 2H), 1.49 (d, J = 6.4 Hz, 6H), 1.32 (t , J = 7.6 Hz, 3H). | 423.3 |
| 111 | 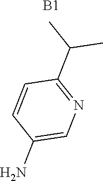 | 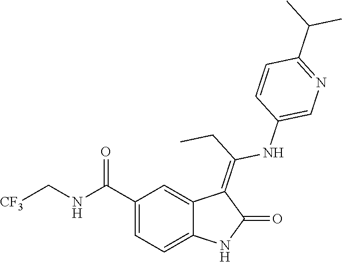 [SMILES: CC/C(Nc1ccc(C(C)C)nc1)=C1/C(=O)Nc2ccc(C(=O)NCC(F)(F)F)cc21] | K | 16 | 12.09 (s, 1H), 10.95 (s, 1H), 8.92 (t, J = 6.0 Hz, 1H), 8.49 (d, J = 2.0 Hz, 1H), 7.82 (s, 1H), 7.74 (dd, J = 8.4, 2.4 Hz, 1H), 7.63 (d, J = 8.0 Hz, 1H), 7.38 (d, J = 8.4 Hz, 1H), 6.97 (d, J = 8.0 Hz, 1H), 4.03- 4.12 (m, 2H), 3.07 (m, 1H), 2.82 (q, J = 6.8 Hz, 2H), 1.27 (d, J = 6.8 Hz, 6H), 1.22 (t, J = 7.2 Hz, 3H). | 433.3 |
| 112 | 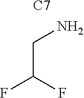 | 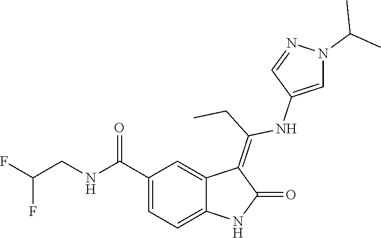 [SMILES: CC/C(Nc1cnn(C(C)C)c1)=C1/C(=O)Nc2ccc(C(=O)NCC(F)F)cc21] | I | 33 | 11.73 (s, 1H), 10.84 (s, 1H), 8.71 (t, J = 5.8 Hz, 1H), 8.01 (s, 1H), 7.80 (s, 1H), 7.60 (dd, J = 8.0, 1.6 Hz, 1H), 7.56 (s, 1H), 6.94 (d, J = 8.1 Hz, 1H), 6.12 (tt, J = 56.3, 4.2 Hz, 1H), 4.50 (m, 1H), 3.65 (m, 2H), 2.82 (q, J = 7.5 Hz, 2H), 1.44 (d, J = 6.6 Hz, 6H), 1.25 (t, J = 7.5 Hz, 3H). | 404.2 |
| 113 | 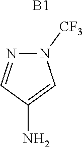 | 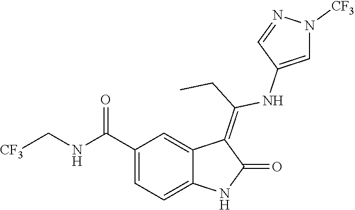 [SMILES: CC/C(Nc1cnn(C(F)(F)F)c1)=C1/C(=O)Nc2ccc(C(=O)NCC(F)(F)F)cc21] | K | 59 | 11.76 (s, 1H), 10.96 (s, 1H), 8.94 (t, J = 6.3 Hz, 1H), 8.75 (s, 1H), 8.18 (s, 1H), 7.84 (d, J = 1.6 Hz, 1H), 7.65 (dd, J = 8.2, 1.5 Hz, 1H), 6.98 (d, J = 8.2 Hz, 1H), 4.08 (m, 2H), 2.86 (q, J = 7.6 Hz, 2H), 1.24 (t, J = 7.5 Hz, 3H). | 448.2 |
| 114 | 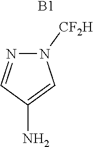 | 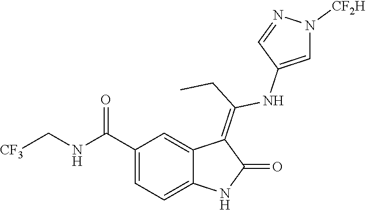 [SMILES: CC/C(Nc1cnn(C(F)F)c1)=C1/C(=O)Nc2ccc(C(=O)NCC(F)(F)F)cc21] | K | 76 | 11.76 (s, 1H), 10.93 (s, 1H), 8.92 (t, J = 6.3 Hz, 1H), 8.51 (s, 1H), 7.98 (s, 1H), 7.83 (t, J = 59.0 Hz, 1H), 7.82 (s, H), 7.64 (dd, J = 8.2, 1.6 Hz, 1H), 6.97 (d, J = 8.2 Hz, 1H), 4.08 (m, 2H), 2.84 (q, J = 7.6 Hz, 2H), 1.24 (t, J = 7.6 Hz, 3H). | 430.2 |
| 115 | 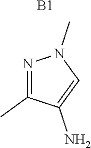 | 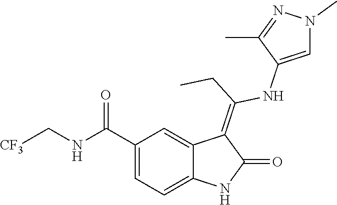 [SMILES: CC/C(Nc1cn(C)nc1C)=C1/C(=O)Nc2ccc(C(=O)NCC(F)(F)F)cc21] | K | 79 | 11.61 (s, 1H), 10.85 (s, 1H), 8.90 (t, J = 6.3 Hz, 1H), 7.86 (s, 1H), 7.81 (d, J = 1.6 Hz, 1H), 7.61 (dd, J = 8.2, 1.6 Hz, 1H), 6.96 (d, J = 8.2 Hz, 1H), 4.03-4.13 (m, 2H), 3.80 (s, 3H), 2.78 (q, J = 7.5 Hz, 2H), 2.10 (s, 3H), 1.20 (t, J = 7.5 Hz, 3H). | 408.2 |
| 116 | 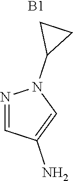 | 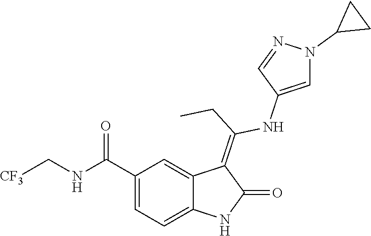 [SMILES: CC/C(Nc1cnn(C2CC2)c1)=C1/C(=O)Nc2ccc(C(=O)NCC(F)(F)F)cc21] | K | 37 | 11.69 (s, 1H), 10.86 (s, 1H), 8.91 (t, J = 6.2 Hz, 1H), 8.03 (s, 1H), 7.80 (s, 1H), 7.61 (d, J = 8.2 Hz, 1H), 7.55 (s, 1H), 6.95 (d, J = 8.1 Hz, 1H), 4.08 (m, 2H), 3.76 (m, 1H), 2.82 (q, J = 7.5 Hz, 2H), 1.24 (t, J = 7.5 Hz, 3H), 1.05- 1.12 (m, 2H), 0.95- 1.01 (m, 2H). | 420.0 |
| 117 | 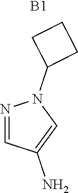 | 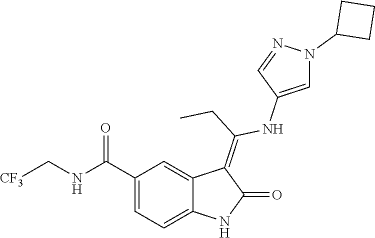 [SMILES: CC/C(Nc1cnn(C2CCC2)c1)=C1/C(=O)Nc2ccc(C(=O)NCC(F)(F)F)cc21] | K | 73 | 11.73 (s, 1H), 10.86 (s, 1H), 8.90 (t, J = 6.3 Hz, 1H), 8.07 (s, 1H), 7.80 (d, J = 1.6 Hz, 1H), 7.61 (dd, J = 8.3, 1.3 Hz, 2H), 6.96 (d, J = 8.2 Hz, 1H), 4.84 (m, 1H), 4.08 (m, 2H), 2.82 (q, J = 7.5 Hz, 2H), 2.35-2.44 (m, 2H), 1.75-1.84 (m, 2H), 1.25 (t, J = 7.5 Hz, 3H). | 434.2 |
| 118 | 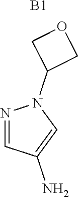 | 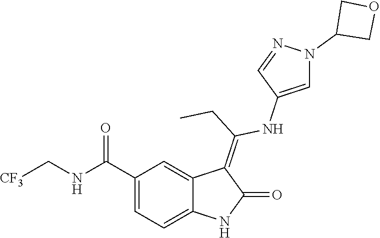 [SMILES: CC/C(Nc1cnn(C2COC2)c1)=C1/C(=O)Nc2ccc(C(=O)NCC(F)(F)F)cc21] | K | 81 | 11.75 (s, 1H), 10.88 (s, 1H), 8.90 (t, J = 6.3 Hz, 1H), 8.15 (s, 1H), 7.81 (d, J = 1.6 Hz, 1H), 7.74 (d, J = 0.8 Hz, 1H), 7.62 (dd, J = 8.2, 1.6 Hz, 1H), 6.96 (d, J = 8.1 Hz, 1H), 5.59 (m, 1H), 4.89-4.97 (m, 4H), 4.08 (m, 2H), 2.83 (q, J = 7.6 Hz, 2H), 1.25 (t, J = 7.5 Hz, 3H). | 436.2 |
| 119 | 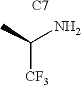 | 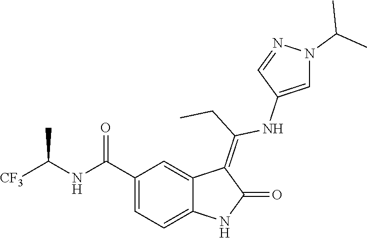 [SMILES: CC/C(Nc1cnn(C(C)C)c1)=C1/C(=O)Nc2ccc(C(=O)N[C@H](C)C(F)(F)F)cc21] | I | 11 | 11.71 (s, 1H), 10.85 (s, 1H), 8.64 (d, J = 8.7 Hz, 1H), 8.01 (s, 1H), 7.78 (s, 1H), 7.61 (d, J = 8.2 Hz, 1H), 7.56 (s, 1H), 6.94 (d, J = 8.1 Hz, 1H), 4.86 (m, 1H), 4.51 (m, 1H), 2.81 (q, J = 7.2 Hz, 2H), 1.44 (d, J = 6.5, Hz, 6H), 1.36 (d, J = 7.0 Hz, 3H), 1.25 (t, J = 7.5 Hz, 3H). | 436.2 |
| 120 | 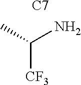 | 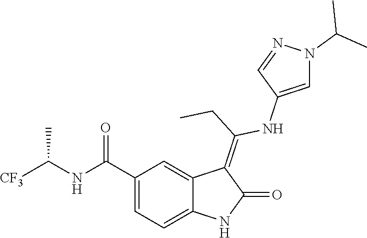 [SMILES: CC/C(Nc1cnn(C(C)C)c1)=C1/C(=O)Nc2ccc(C(=O)N[C@@H](C)C(F)(F)F)cc21] | I | 6 | 11.71 (s, 1H), 10.85 (s, 1H), 8.64 (d, J = 8.8 Hz, 1H), 8.01 (s, 1H), 7.78 (s, 1H), 7.61 (d, J = 8.1 Hz, 1H), 7.56 (s, 1H), 6.94 (d, J = 8.2 Hz, 1H), 4.85 (m, 1H), 4.50 (m, 1H), 2.81 (q, J = 7.2 Hz, 2H), 1.44 (d, J = 6.7 Hz, 6H), 1.36 (d, J = 7.1 Hz, 3H), 1.25 (t, J = 7.4 Hz, 3H). | 436.2 |
| 121 | 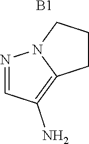 | 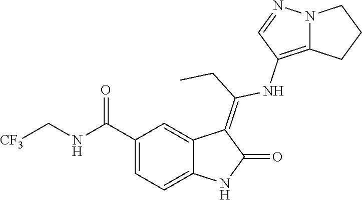 [SMILES: CC/C(Nc1cnn2c1CCC2)=C1/C(=O)Nc2ccc(C(=O)NCC(F)(F)F)cc21] | K | 74 | 11.43 (s, 1H), 10.78 (s, 1H), 8.83 (t, J = 6.2 Hz, 1H), 7.73 (s, 1H), 7.54 (d, J = 8.2 Hz, 1H), 7.47 (s, 1H), 6.89 (d, J = 8.1 Hz, 1H), 3.97- 4.08 (m, 4H), 2.80 (m, 2H), 2.73 (q, J = 7.6 Hz, 2H), 2.51 (m, 2H), 1.12 (t, J = 7.5 Hz, 3H). | 420.2 |
| 122 | 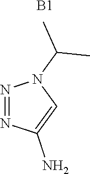 | 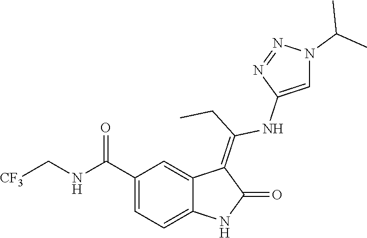 [SMILES: CC/C(Nc1cn(C(C)C)nn1)=C1/C(=O)Nc2ccc(C(=O)NCC(F)(F)F)cc21] | K | 47 | 12.25 (s, 1H), 10.99 (s, 1H), 8.95 (t, J = 6.2 Hz, 1H), 8.32 (s, 1H), 7.87 (s, 1H), 7.66 (d, J = 8.2 Hz, 1H), 6.98 (d, J = 8.1 Hz, 1H), 4.83 (m, 1H), 4.08 (m, 2H), 2.98 (q, J = 7.4 Hz, 2H), 1.54 (d, J = 6.6 Hz, 6H), 1.31 (t, J = 7.5 Hz, 3H). | 423.2 |
| 123 | 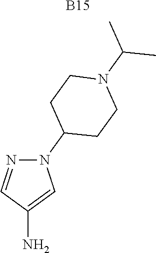 | 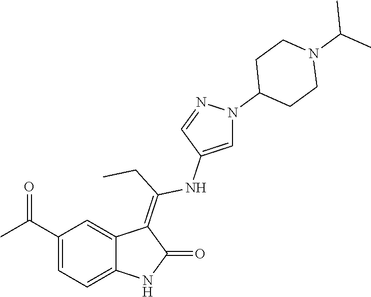 [SMILES: CC/C(Nc1cnn(C2CCN(C(C)C)CC2)c1)=C1/C(=O)Nc2ccc(C(C)=O)cc21] | K | 60 | 11.65 (s, 1H), 10.94 (s, 1H), 8.02 (s, 1H), 7.82 (s, 1H), 7.70 (dd, J = 8.2, 1.5 Hz, 1H), 7.56 (s, 1H), 6.97 (d, J = 8.2 Hz, 1H), 4.10 (m, 1H), 2.87 (m, 2H), 2.70- 2.79 (m, 3H), 2.54 (s, 3H), 2.25 (m, 2H), 2.01 (m, 2H), 1.89 (m, 2H), 1.25 (t, J = 7.6 Hz, 3H), 0.98 (d, J = 6.6 Hz, 6H). | 422.3 |
| 124 | 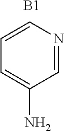 | 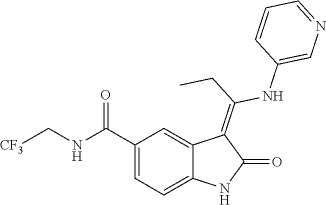 [SMILES: CC/C(Nc1cccnc1)=C1/C(=O)Nc2ccc(C(=O)NCC(F)(F)F)cc21] | K | 65 | 12.14 (s, 1H), 10.99 (s, 1H), 8.96 (br t, J = 6.3 Hz, 1H), 8.60 (br s 1H), 8.54 (d, J = 4.7 Hz, 1H), 7.80-7.87 (m, 2H), 7.66 (d, J = 8.2 Hz, 1H), 7.52 (br t, J = 4.7 Hz, 1H), 6.99 (d, J = 8.2 Hz, 1H), 4.08 (m, 2H), 2.85 (q, J = 7.0 Hz, 2H), 1.24 (t, J = 7.0 Hz, 3H). | 391.2 |
| 125 | 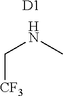 | 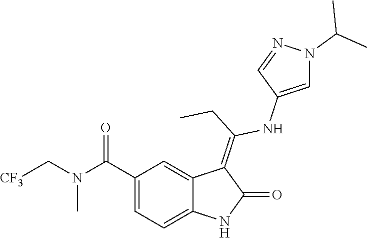 [SMILES: CC/C(Nc1cnn(C(C)C)c1)=C1/C(=O)Nc2ccc(C(=O)N(C)CC(F)(F)F)cc21] | L (EDC) | 51 | 11.74 (s, 1H), 10.80 (s, 1H), 8.01 (s, 1H), 7.56 (s, 1H), 7.29 (s, 1H), 7.09 (d, J = 8.0 Hz, 1H), 6.96 (d, J = 7.9 Hz, 1H), 4.51 (m, 1H), 4.33 (m, 2H), 3.10 (s, 3H), 2.74 (q, J = 7.5 Hz, 2H), 1.44 (d, J = 6.7 Hz, 6H), 1.23 (t, J = 7.5 Hz, 3H). | 436.2 |
| 126 | 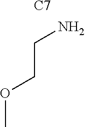 | 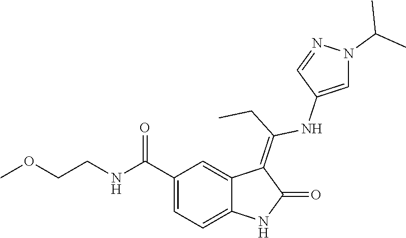 [SMILES: CC/C(Nc1cnn(C(C)C)c1)=C1/C(=O)Nc2ccc(C(=O)NCCOC)cc21] | J | 19 | 11.71 (s, 1H), 10.78 (s, 1H), 8.36 (t, J = 5.6 Hz, 1H), 8.00 (s, 1H), 7.76 (s, 1H), 7.53-7.57 (m , 2H), 6.90 (d, J = 8.0 Hz, 1H), 4.51 (m, 1H), 3.39-3.46 (m, 4H), 3.26 (s, 3H), 2.81 (q, J = 7.6 Hz, 2H), 1.42 (d, J = 6.8 HZ, 6H), 1.24 (t, J = 7.6 Hz, 3H). | 398.3 |
| 127 | 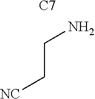 | 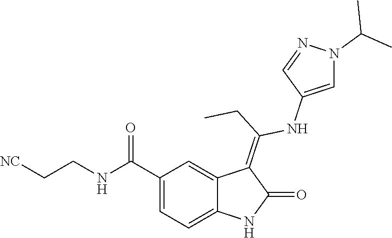 [SMILES: CC/C(Nc1cnn(C(C)C)c1)=C1/C(=O)Nc2ccc(C(=O)NCCC#N)cc21] | J | 17 | 11.72 (s, 1H), 10.81 (s, 1H), 8.67 ( t, J = 5.6 Hz, 1H), 8.00 (s, 1H), 7.77 (s, 1H), 7.55 (m, 2H), 6.93 (d, J = 8.4 Hz, 1H), 4.49 ( m, 1H), 3.47 (q, J = 7.6 Hz, 2H), 2.76-2.84 (m, 4H), 1.43 (d, J = 6.4 Hz, 6H), 1.24 (t, J = 7.6 Hz, 3H). | 393.4 |
| 128 | 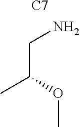 | 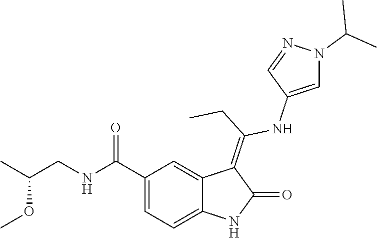 [SMILES: CC/C(Nc1cnn(C(C)C)c1)=C1/C(=O)Nc2ccc(C(=O)NC[C@@H](C)OC)cc21] | J | 17 | 11.71 (s, 1H), 10.77 (s, 1H), 8.33 (t, J = 5.6 Hz, 1H), 8.00 (s, 1H), 7.76 (s, 1H), 7.55 (m, 2H), 6.90 (d, J = 8.0 Hz, 1H), 4.50 (m, 1H), 3.48 (m, 1H), 3.21-3.27 (m, 2H), 3.27 (s, 3H), 2.82 (q, J = 7.2 Hz, 2H), 1.42 (d, J = 6.8 Hz, 6H), 1.24 (t, J = 7.2 Hz, 3H), 1.07 (d, J = 6.4 Hz, 3H). | 412.3 |
| 129 | 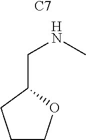 | 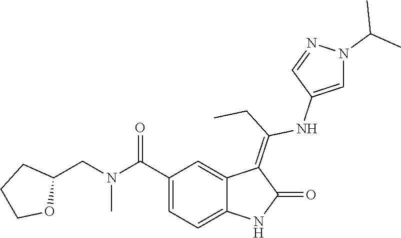 [SMILES: CC/C(Nc1cnn(C(C)C)c1)=C1/C(=O)Nc2ccc(C(=O)N(C)C[C@H]3CCCO3)cc21] | J | 44 | 11.70 (S, 1H), 10.72 (s, 1H), 7.99 (s, 1H), 8.00 (s, 1H), 7.74 (s, 1H), 7.54 (s, 1H), 7.24 (s, 1H), 7.02 (d, J = 8.0 Hz, 1H), 6.89 (d, J = 8.0 Hz, 1H), 4.49 (m, 1H), 4.05 (br m, 1H), 3.75 (br m, 1H), 3.61 (br m, 2H), 3.39 (m, 1H), 3.00 (s, 1H), 2.71 (q, J = 7.2 Hz, 2H), 1.60-2.00 (br m, 4H), 1.42 (d, J = 6.4 Hz, 6H), 1.22 (t, J = 7.2 Hz, | 438.2 |
| | | | | | 3H). | |
| 130 | 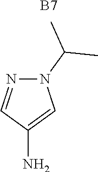 | 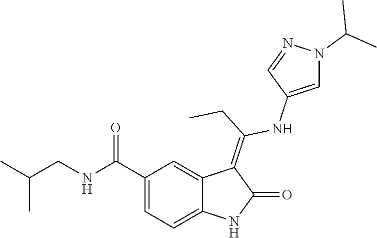 [SMILES: CC/C(Nc1cnn(C(C)C)c1)=C1/C(=O)Nc2ccc(C(=O)NCC(C)C)cc21] | K | 33 | 11.71 (s, 1H), 10.75 (s, 1H), 8.28 (br t, J = 6.0 Hz, 1H), 7.99 (br s, 1H), 7.75 (br s, 1H), 7.53 (br m, 2H), 6.90 (br d, J = 8.0 Hz, 1H), 4.50 (m, 1H), 3.07 (t, J = 6 Hz, 2H), 2.82 (q, J = 7.0 Hz, 2H), 1.84 (m, 1H), 1.43 (d, J = 6.4 Hz, 6H), 1.24 (t, J = 6.8 Hz, 3H), 0.87 (d, J = 6.4 Hz, 6H). | 396.4 |
| 131 | 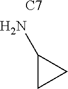 | 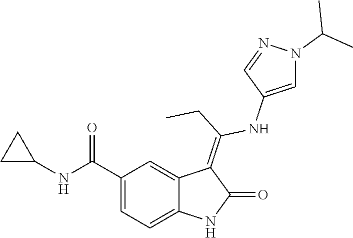 [SMILES: CC/C(Nc1cnn(C(C)C)c1)=C1/C(=O)Nc2ccc(C(=O)NC3CC3)cc21] | J | 9 | 11.70 (s, 1H), 10.76 (s, 1H), 8.25 (br d, J = 3.6 Hz, 1H), 7.99 (s, 1H), 7.73 (s, 1H), 7.55 (s, 1H), 7.49 (br d, J = 8.0 Hz, 1H), 6.87 (d, J = 8.4 Hz, 1H), 4.49 (m, 1H), 2.77-2.83 (m, 3H), 1.42 (d, J = 6.8 Hz, 6H), 1.24 (t, J = 7.2 Hz, 3H), 0.63-0.69 (m, 2H), 0.53-0.55 (m, 2H). | 380.4 |
| 132 | 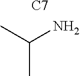 | 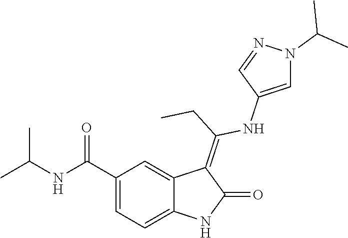 [SMILES: CC/C(Nc1cnn(C(C)C)c1)=C1/C(=O)Nc2ccc(C(=O)NC(C)C)cc21] | J | 18 | 11.69 (s, 1H), 10.76 (s, 1H), 8.01 (d, J = 8.0 Hz, 1H), 8.00 (s, 1H), 7.74 (s, 1H), 7.53 (m, 2H), 6.88 (d, J = 8.0 Hz, 1H), 4.49 (m, 1H), 4.09 (m, 1H), 2.79 (q, J = 7.6 Hz, 2H), 1.42 (d, J = 6.8 Hz, 6H), 1.24 (t, J = 7.6 Hz, 3H), 1.15 (d, J = 6.8 Hz, 6H). | 382.3 |
| 133 | C7 MeNH₂ | 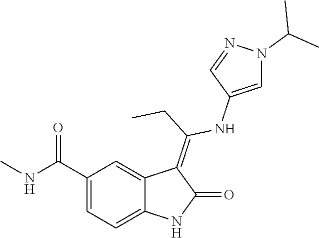 [SMILES: CC/C(Nc1cnn(C(C)C)c1)=C1/C(=O)Nc2ccc(C(=O)NC)cc21] | J | 5 | 11.72 (s, 1H), 10.76 (s, 1H), 8.24 (q, J = 4.4 Hz, 1H), 7.99 (s, 1H), 7.74 (s, 1H), 7.55 (s, 1H), 7.51 (d, J = 8.0 Hz, 1H), 6.90 (d, J = 8.0 Hz, 1H), 4.49 (m, 1H), 2.82 (q, J = 7.6 Hz, 2H), 2.76 (d, J = 4.4 Hz, 3H), 1.43 (d, J = 6.4 Hz, 6H), 1.24 (t, J = 7.6 Hz, 3H). | 354.3 |
| 134 | 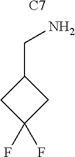 | 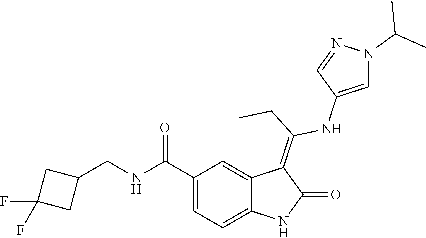 [SMILES: CC/C(Nc1cnn(C(C)C)c1)=C1/C(=O)Nc2ccc(C(=O)NCC3CC(F)(F)C3)cc21] | I | 14 | 11.72 (s, 1H), 10.80 (s, 1H), 8.47 (t, J = 5.8 Hz, 1H), 8.00 (s, 1H), 7.76 (s, 1H), 7.52-7.57 (m, 2H), 6.92 (d, J = 8.1 Hz, 1H), 4.50 (m, 1H), 3.38 (t, J = 5.8 Hz, 2H), 2.81 (q, J = 7.5 Hz, 2H), 2.56-2.66 (m, 2H), 2.31-2.45 (m, 3H), 1.44 (d, J = 6.6 Hz, 6H), 1.24 (t, J = 7.5 Hz, 3H). | 444.2 |
| 135 | 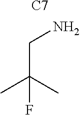 | 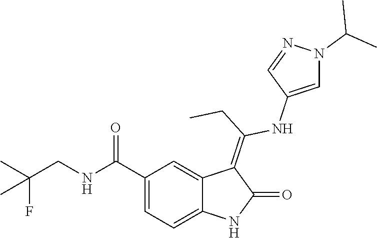 [SMILES: CC/C(Nc1cnn(C(C)C)c1)=C1/C(=O)Nc2ccc(C(=O)NCC(C)(C)F)cc21] | I | 47 | 11.73 (s, 1H), 10.80 (s, 1H), 8.46 (t, J = 6.3 Hz, 1H), 8.01 (s, 1H), 7.80 (s, 1H), 7.60 (d, J = 8.2 Hz, 1H), 7.56 (s, 1H), 6.93 (d, J = 8.1 Hz, 1H), 4.51 (m, 1H), 3.48 (m, 2H), 2.84 (q, J = 7.6 Hz, 2H), 1.45 (d, J = 7.6 Hz, 6H), 1.33 (d, J = 20.0 Hz, 6H), 1.25 (t, J = 7.5 Hz, 3H). | 414.2 |
| 136 | 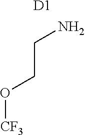 | 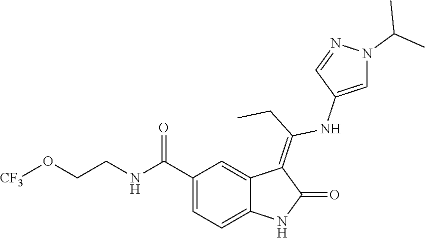 [SMILES: CC/C(Nc1cnn(C(C)C)c1)=C1/C(=O)Nc2ccc(C(=O)NCCOC(F)(F)F)cc21] | L | 32 | 11.73 (s, 1H), 10.81 (s, 1H), 8.57 (t, J = 5.6 Hz, 1H), 8.00 (s, 1H), 7.77 (s, 1H), 7.53-7.58 (m, 2H), 6.93 (d, J = 7.7 Hz, 1H), 4.50 (m, 1H), 4.19 (t, J = 5.4 Hz, 2H), 3.56 (q, J = 5.5 Hz, 2H), 2.82 (q, J = 7.5 Hz, 2H), 1.44 (d, J = 6.7, 6H), 1.24 (t, J = 7.5 Hz, 3H). | 452.2 |
| 137 | 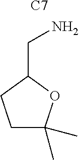 | 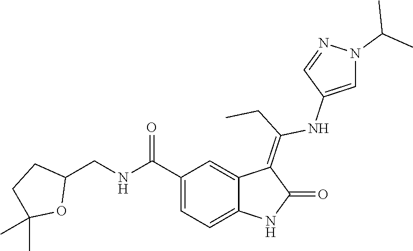 [SMILES: CC/C(Nc1cnn(C(C)C)c1)=C1/C(=O)Nc2ccc(C(=O)NCC3CCC(C)(C)O3)cc21] | I | 13 | 11.72 (s, 1H), 10.78 (s, 1H), 8.32 (t, J = 5.9 Hz, 1H), 8.00 (s, 1H), 7.77 (d, J = 1.6 Hz, 1H), 7.55 (m, 2H), 6.91 (d, J = 8.1 Hz, 1H), 4.50 (m, 1H), 4.04 (m, 1H), 3.35 (m, 1H), 3.26 (m, 1H), 2.82 (m, 2H), 1.96 (m, 1H), 1.63-1.76 (m, 3H), 1.44 (d, J = 6.7 Hz, 6H), 1.24 (t, J = 7.6 Hz, 3H), 1.20 (s, 3H), 1.14 (s, 3H). | 452.2 |
| 138 | 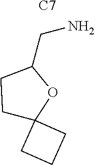 | 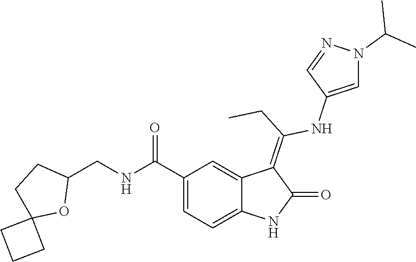 [SMILES: CC/C(Nc1cnn(C(C)C)c1)=C1/C(=O)Nc2ccc(C(=O)NCC3CCC4(CCC4)O3)cc21] | I | 23 | 11.73 (s, 1H), 10.78 (s, 1H), 8.32 (t, J = 5.9 Hz, 1H), 8.00 (s, 1H), 7.77 (s, 1H), 7.52-7.63 (m, 2H), 6.91 (d, J = 8.1 Hz, 1H), 4.50 (m, 1H), 4.03 (m, 1H), 3.31 (m, 1H), 3.23 (m, 1H), 2.81 (m, 2H), 2.03-2.22 (m, 2H), 1.80-1.97 (m, 5H), 1.52-1.70 (m, 2H), 1.38-1.51 (m, 7H), 1.24 (t, J = 7.5 Hz, 3H). | 464.2 |
| 139 | 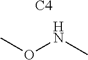 | 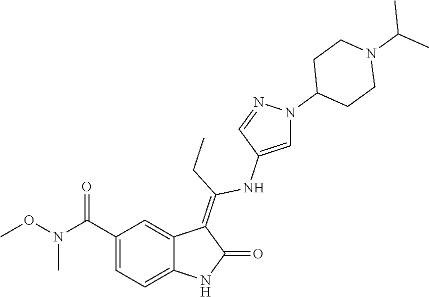 [SMILES: CC/C(Nc1cnn(C2CCN(C(C)C)CC2)c1)=C1/C(=O)Nc2ccc(C(=O)N(C)OC)cc21] | I | 5 | 11.66 (s, 1H), 10.78 (s, 1H), 8.01 (s, 1H), 7.57 (s, 1H), 7.55 (s, 1H), 7.33 (dd, J = 8.1, 1.4 Hz, 1H), 6.92 (d, J = 8.1 Hz, 1H), 4.09 (br m, 1H), 3.55 (s, 3H), 3.42 (s, 3H), 2.87 (m, 2H), 2.73 (q, J = 7.2 Hz, 3H), 2.24 (m, 2H), 2.00 (m, 2H), 1.89 (m, 2H), 1.22 (t, J = 7.6 Hz, 3H), 0.98 (d, J = 6.5 Hz, 6H). | 467.2 |
| 140 | 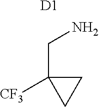 | 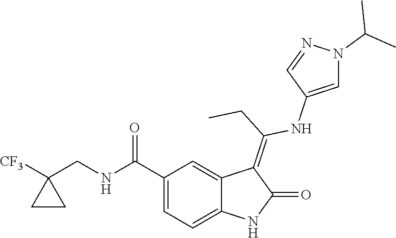 [SMILES: CC/C(Nc1cnn(C(C)C)c1)=C1/C(=O)Nc2ccc(C(=O)NCC3(C(F)(F)F)CC3)cc21] | L | 58 | 11.72 (s, 1H), 10.80 (s, 1H), 8.42 (t, J = 6.2 Hz, 1H), 8.00 (s, 1H), 7.74 (s, 1H), 7.56 (s, 1H), 7.52 (dd, J = 8.1, 1.5 Hz, 1H), 6.93 (d, J = 8.1 Hz, 1H), 4.50 (m, 1H), 3.60 (d, J = 6.1 Hz, 2H), 2.82 (q, J = 7.6 Hz, 2H), 1.44 (d, J = 6.7 Hz, 6H), 1.24 (t, J = 7.5 Hz, 3H), 0.87- 0.96 (m, 4H). | 462.2 |
| 141 | 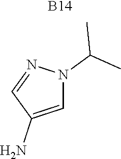 | 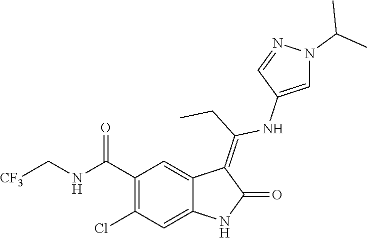 [SMILES: CC/C(Nc1cnn(C(C)C)c1)=C1/C(=O)Nc2cc(Cl)c(C(=O)NCC(F)(F)F)cc21] | K | 80 | 11.70 (s, 1H), 10.85 (s, 1H), 8.96 (t, J = 6.4 Hz, 1H), 8.01 (s, 1H), 7.56 (s, 1H), 7.26 (s, 1H), 6.94 (s, 1H), 4.50 (m, 1H), 4.00-4.10 (m, 2H), 2.73 (q, J = 7.6 Hz, 2H), 1.43 (d, J = 6.7 Hz, 6H), 1.23 (t, J = 7.6 Hz, 3H). | 456 |
| 142 | 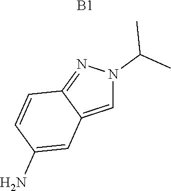 | 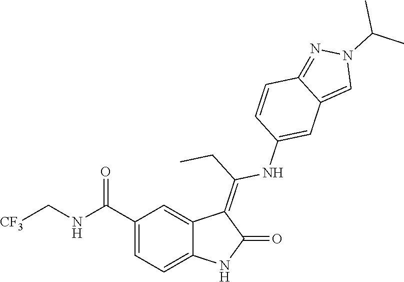 [SMILES: CC/C(Nc1ccc2nn(C(C)C)cc2c1)=C1/C(=O)Nc2ccc(C(=O)NCC(F)(F)F)cc21] | K | 40 | 12.14 (s, 1H), 10.89 (s, 1H), 8.90 (t, J = 6.3 Hz, 1H), 8.47 (s, 1H), 7.82 (s, 1H), 7.72 (d, J = 8.9 Hz, 1H), 7.68 (d, J = 2.0 Hz, 1H), 7.62 (d, J = 8.1 Hz, 1H), 7.18 (dd, J = 9.0, 2.0 Hz, 1H), 6.98 (d, J = 8.1 Hz, 1H), 4.86 (m, 1H), 4.08 (m, 2H), 2.84 (q, J = 7.5 Hz, 2H), 1.57 (d, J = 6.7 Hz, 6H), 1.24 (t, J = 7.5 Hz, 3H). | 472.2 |
| 143 | 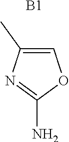 | 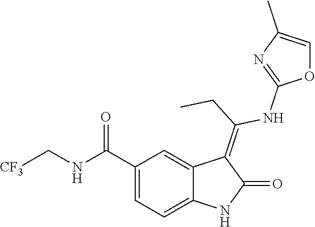 [SMILES: CC/C(Nc1nc(C)co1)=C1/C(=O)Nc2ccc(C(=O)NCC(F)(F)F)cc21] | K | 15 | 13.06 (s, 1H), 11.21 (s, 1H), 9.00 (t, J = 6.3 Hz, 1H), 7.95 (s, 1H), 7.73 (d, J = 8.2 Hz, 1H), 7.60 (s, 1H), 7.02 (d, J = 8.1 Hz, 1H), 4.09 (m, 2H), 3.42 (q, J = 7.5 Hz, 2H), 2.09 (s, 3H), 1.35 (t, J = 7.4 Hz, 3H). | 395.2 |
| 144 | 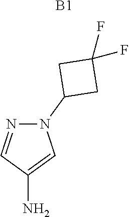 | 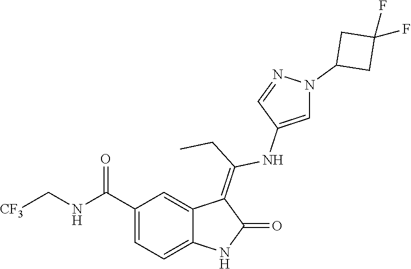 [SMILES: CC/C(Nc1cnn(C2CC(F)(F)C2)c1)=C1/C(=O)Nc2ccc(C(=O)NCC(F)(F)F)cc21] | K | 54 | 11.75 (s, 1H), 10.88 (s, 1H), 8.91 (t, J = 6.0 Hz, 1H), 8.12 (s, 1H), 7.80 (s, 1H), 7.71 (s, 1H), 7.62 (dd, J = 8.0 Hz, 1.2 Hz, 1H), 6.94 (d, J = 8.0 Hz, 1H), 4.93 (m, 1H), 4.07 (m, 2H), 3.12-3.21 (m, 4H), 2.83 (q, J = 7.2 Hz, 2H), 1.24 (t, J = 7.6 Hz, 3H). | 470.1 |
| 145 | 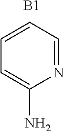 | 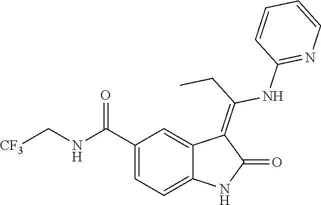 [SMILES: CC/C(Nc1ccccn1)=C1/C(=O)Nc2ccc(C(=O)NCC(F)(F)F)cc21] | K | 38 | 12.74 (s, 1H), 11.07 (s, 1H), 8.98 (t, J = 6.2 Hz, 1H), 8.42 (d, J = 4.9 Hz, 1H), 7.95 (s, 1H), 7.84 (t, J = 7.9 Hz, 1H), 7.69 (d, J = 8.2 Hz, 1H), 7.12-7.20 (m, 2H), 7.00 (d, J = 8.1 Hz, 1H), 4.09 (m, 2H), 3.45 (q, J = 7.4 Hz, 2H), 1.43 (t, J = 7.4 Hz, 3H). | 391.2 |
| 146 | 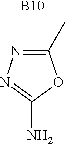 | 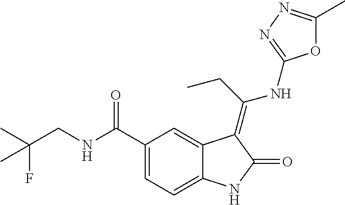 [SMILES: CC/C(Nc1nnc(C)o1)=C1/C(=O)Nc2ccc(C(=O)NCC(C)(C)F)cc21] | K | 62 | 13.10 (s, 1H), 11.25 (s, 1H), 8.59 (t, J = 6.2 Hz, 1H), 7.95 (s, 1H), 7.75 (d, J = 8.2 Hz, 1H), 7.00 (d, J = 8.1 Hz, 1H), 3.49 (dd, J = 20.1, 6.2 Hz, 2H), 3.37 (q, J = 7.5 Hz, 2H), 2.46 (s, 3H), 1.35 (t, J = 7.5 Hz, 3H), 1.33 (d, J = 20.0 Hz, 6H). | 388.2 |
| 147 | 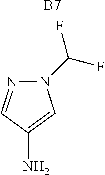 | 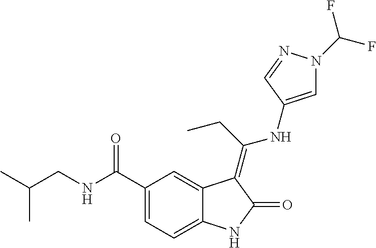 [SMILES: CC/C(Nc1cnn(C(F)F)c1)=C1/C(=O)Nc2ccc(C(=O)NCC(C)C)cc21] | K | 14 | 11.74 (s, 1H), 10.85 (s, 1H), 8.50 (s, 1H), 8.33 (t, J = 6.0 Hz, 1H), 7.99 (s, 1H), 7.82 (t, J = 58.9 Hz, 1H), 7.78 (s, 1H), 7.58 (d, J = 8.1 Hz, 1H), 6.93 (d, J = 8.1 Hz, 1H), 3.08 (t, J = 6.4 Hz, 2H), 2.84 (q, J = 7.6 Hz, 2H), 1.85 (m, 1H), 1.25 (t, J = 7.5 Hz, 3H), 0.90 (d, J = 6.7 Hz, 6H). | 404.2 |
| 148 | 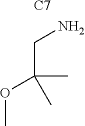 | 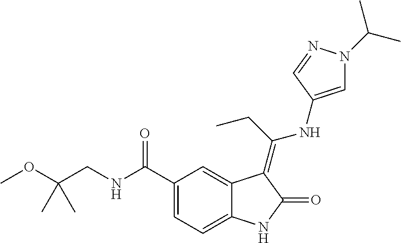 [SMILES: CC/C(Nc1cnn(C(C)C)c1)=C1/C(=O)Nc2ccc(C(=O)NCC(C)(C)OC)cc21] | J | 36 | 11.70 (s, 1H), 10.78 (s, 1H), 8.06 (t, J = 6.0 Hz, 1H), 8.00 (s, 1H), 7.74 (s, 1H), 7.55 (s, 1H), 7.54 (m, 1H), 6.90 (d, J = 8.4 Hz, 1H), 4.49 (m, 1H), 3.32 (s, 2H), 3.15 (s, 3H), 2.80 (q, J = 7.2 Hz, 2H), 1.42 (d, J = 6.8 Hz, 6H), 1.24 (t, J = 7.2 Hz, 3H), 1.10 (s, 6H). | 426.2 |
| 149 | 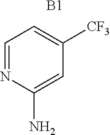 | 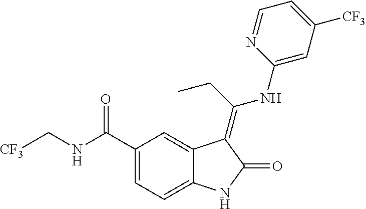 [SMILES: CC/C(Nc1cc(C(F)(F)F)ccn1)=C1/C(=O)Nc2ccc(C(=O)NCC(F)(F)F)cc21] | K | 8 | 12.80 (s, 1H), 11.14 (s, 1H), 8.98 (t, J = 6.4 Hz, 1H), 8.6 ( d, J = 5.2 Hz, 1H), 7.96 (s, 1H), 7.69 ( d, J = 8.4 Hz, 1H), 7.45 (m, 2H), 6.99 (d, J = 8.4 Hz, 1H), 3.99- 4.14 (m, 2H) , 3.43 (q, J = 7.2 Hz, 2H), 1.42 (t, J = 7.6 Hz, 3H). | 458.1 |
| 150 | 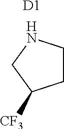 | 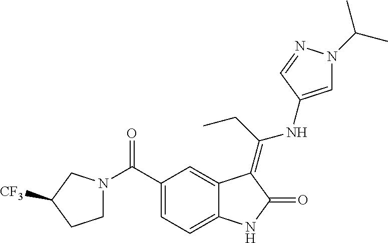 [SMILES: CC/C(Nc1cnn(C(C)C)c1)=C1/C(=O)Nc2ccc(C(=O)N3CC[C@@H](C(F)(F)F)C3)cc21] | L | 70 | 11.71 (s, 1H), 10.77 (s, 1H), 8.00 (s, 1H), 7.55 (s, 1H), 7.40 (s, 1H), 7.21 (d, J = 8.2 Hz, 1H), 6.93 (d, J = 7.9 Hz, 1H), 4.50 (m, 1H), 3.75 (m, 1H), 3.53- 3.68 (br m, 3H), 3.29 (br m, 1H), 2.68-2.81 (m, 2H), 2.17 (br m, 1H), 1.99 (br m, 1H), 1.43 (d, J = 6.6 Hz, 6H), 1.23 (t, J = 7.5 Hz, 3H). | 462.2 |
| 151 | 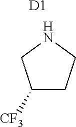 | 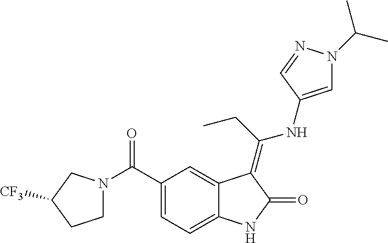 [SMILES: CC/C(Nc1cnn(C(C)C)c1)=C1/C(=O)Nc2ccc(C(=O)N3CC[C@H](C(F)(F)F)C3)cc21] | L | 19 | 11.71 (s, 1H), 10.77 (s, 1H), 8.00 (s, 1H), 7.55 (s, 1H), 7.40 (s, 1H), 7.21 (d, J = 8.0 Hz, 1H), 6.93 (d, J = 8.0 Hz, 1H), 4.50 (m, 1H), 3.75 (m, 1H), 3.53- 3.67 (br m, 3H), 3.29 (br m, 1H), 2.68-2.80 (m, 2H), 2.17 (br m, 1H), 1.99 (br m, 1H), 1.43 (d, J = 6.7 Hz, 6H), 1.23 (t, J = 7.5 Hz, 3H). | 462.2 |
| 152 | 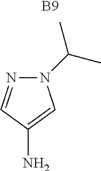 | 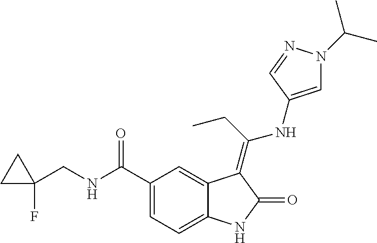 [SMILES: CC/C(Nc1cnn(C(C)C)c1)=C1/C(=O)Nc2ccc(C(=O)NCC3(F)CC3)cc21] | K | 49 | 11.72 (s, 1H), 10.80 (s, 1H), 8.62 (t, J = 6.0 Hz, 1H), 8.00 (s, 1H), 7.79 (s, 1H), 7.60 (dd, J = 8.1, 1.5 Hz, 1H), 7.56 (s, 1H), 6.93 (d, J = 10 Hz, 1H), 4.50 (m, 1H), 3.70 (dd, J = 20.4, 5.8 Hz, 2H), 2.82 (q, J = 7.5 Hz, 2H), 1.44 (d, J = 6.7 Hz, 6H), 1.25 (t, J = 7.5 Hz, 3H), 0.93- 1.02 (m, 2H), 0.77- 0.84 (m, 2H). | 412.2 |
| 153 | 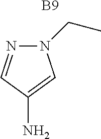 | 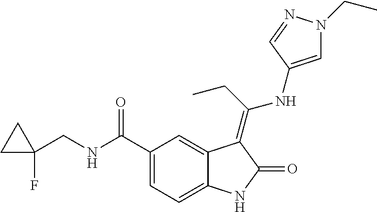 [SMILES: CC/C(Nc1cnn(CC)c1)=C1/C(=O)Nc2ccc(C(=O)NCC3(F)CC3)cc21] | K | 44 | 11.71 (s, 1H), 10.80 (s, 1H), 8.62 (t, J = 6.0 Hz, 1H), 7.98 (s, 1H), 7.79 (s, 1H), 7.60 (d, J = 8.2 Hz, 1H), 7.56 (s, 1H), 6.93 (d, J = 7.9 Hz, 1H), 4.15 (m, 2H), 3.70 (dd, J = 20.4, 5.8 Hz, 2H), 2.82 (q, J = 7.5 Hz, 2H), 1.39 (m, 3H), 1.24 (t, J = 7.5 Hz, 3H), 0.93-1.02 (m, 2H), 0.77-0.83 (m, 2H). | 398.2 |
| 154 | 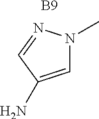 | 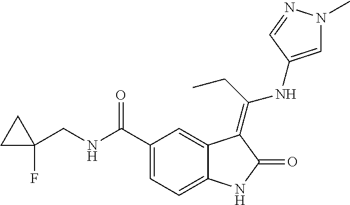 [SMILES: CC/C(Nc1cnn(C)c1)=C1/C(=O)Nc2ccc(C(=O)NCC3(F)CC3)cc21] | K | 59 | 11.69 (s, 1H), 10.80 (s, 1H), 8.62 (t, J = 6.1 Hz, 1H), 7.94 (s, 1H), 7.79 (s, 1H), 7.60 (d, J = 8.2 Hz, 1H), 7.55 (s, 1H), 6.92 (d, J = 8.1 Hz, 1H), 3.86 (s, 3H), 3.70 (dd, J = 20.4, 5.8 Hz, 2H), 2.82 (q, J = 7.5 Hz, 2H), 1.24 (t, J = 7.5 Hz, 3H), 0.93-1.02 (m, 2H), 0.77-0.84 (m, 2H). | 384.2 |
| 155 | 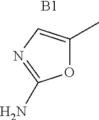 | 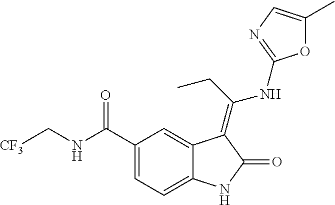 [SMILES: CC/C(Nc1ncc(C)o1)=C1/C(=O)Nc2ccc(C(=O)NCC(F)(F)F)cc21] | K | 5 | 13.04 (s, 1H), 11.21 (s, 1H), 8.99 (t, J = 6.3 Hz, 1H), 7.94 (s, 1H), 7.72 (d, J = 8.2 Hz, 1H), 7.01 (d, J = 8.1 Hz, 1H), 6.80 (d, J = 1.6 Hz, 1H), 4.09 (m, 2H), 3.41 (q, J = 7.4 Hz, 2H), 2.28 (s, 3H), 1.34 (t, J = 7.4 Hz, 3H). | 395.2 |
| 156 | 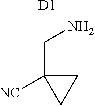 | 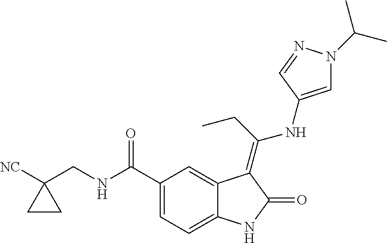 [SMILES: CC/C(Nc1cnn(C(C)C)c1)=C1/C(=O)Nc2ccc(C(=O)NCC3(C#N)CC3)cc21] | L | 65 | 11.73 (s, 1H), 10.83 (s, 1H), 8.71 (t, J = 6.0 Hz, 1H), 8.01 (s, 1H), 7.79 (s, 1H), 7.54-7.61 (m, 2H), 6.95 (dd, J = 8.0, 1.0 Hz, 1H), 4.50 (m, 1H), 3.43 (d, J = 5.9 Hz, 2H), 2.82 (q, J = 7.5 Hz, 2H), 1.44 (d, J = 6.7, 6H), 1.26 (t, J = 7.5 Hz, 3H), 1.10-1.22 (m, 4H). | 419.2 |
| 157 | 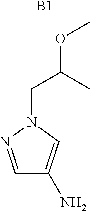 | 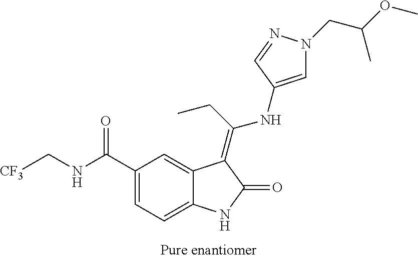 [SMILES: CC/C(Nc1cnn(CC(C)OC)c1)=C1/C(=O)Nc2ccc(C(=O)NCC(F)(F)F)cc21] | K | 11 | 11.69 (s, 1H), 10.87 (s, 1H), 8.90 (t, J = 6.4 Hz, 1H), 7.93 (s, 1H), 7.80 (s, 1H), 7.60 (d, J = 8.8 Hz, 1H), 7.58 (s, 1H), 6.94 (d, J = 8.4 Hz, 1H), 4.03-4.17 (m, 4H), 3.69 (m, 1H), 3.20 (s, 3H), 2.82 (q, J = 7.2 Hz, 2H), 1.22 (t, J = 7.2 Hz,3H), 1.06 (d, J = 6.4 Hz, 3H). | 452.1 |
| 158 | 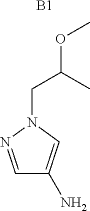 | 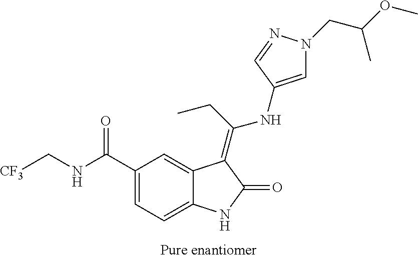 [SMILES: CC/C(Nc1cnn(CC(C)OC)c1)=C1/C(=O)Nc2ccc(C(=O)NCC(F)(F)F)cc21] | K | 12 | 11.69 (s, 1H), 10.87 (s, 1H), 8.90 (t, J = 6.4 Hz, 1H), 7.93 (s, 1H), 7.80 (s, 1H), 7.60 (d, J = 8.8 Hz, 1H), 7.58 (s, 1H), 6.94 (d, J = 8.4 Hz, 1H), 4.03-4.17 (m, 4H), 3.69 (m, 1H), 3.20 (s, 3H), 2.82 (q, J = 7.2 Hz, 2H), 1.22 (t, J = 7.2 Hz, 3H), 1.06 (d, J = 6.4 Hz, 3H). | 452.2 |
| 159 | 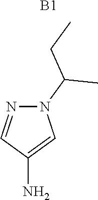 | 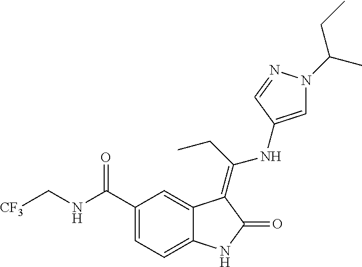 [SMILES: CC/C(Nc1cnn(C(C)CC)c1)=C1/C(=O)Nc2ccc(C(=O)NCC(F)(F)F)cc21] | K | 20 | 11.71 (s, 1H), 10.85 (s, 1H), 8.89 (t, J = 6.0 Hz, 1H), 8.01 (s, 1H), 7.80 (s, 1H), 7.61 (dd, J = 8.0, 1.2 Hz, 1H), 7.59 (s, 1H), 6.94 (d, J = 8.0 Hz, 1H), 4.25 (m, 1H), 4.03-4.12 (m, 2H), 2.81 (q, J = 7.6 Hz, 2H), 1.71-1.85 (m, 2H), 1.42 ( d, J = 6.4 Hz, 3H), 1.23 (t, J = 7.2 Hz, 3H), 0.73 (t, J = 7.6 Hz, 3H). | 436.4 |
| 160 | 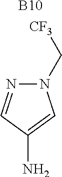 | 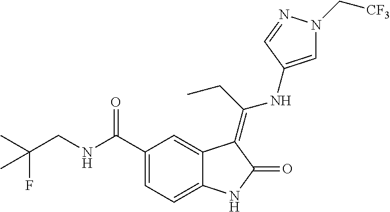 [SMILES: CC/C(Nc1cnn(CC(F)(F)F)c1)=C1/C(=O)Nc2ccc(C(=O)NCC(C)(C)F)cc21] | K | 49 | 11.71 (s, 1H), 10.83 (s, 1H), 8.46 (t, J = 6.3 Hz, 1H), 8.11 (s, 1H), 7.80 (s, 1H), 7.75 (s, 1H), 7.61 (d, J = 8.1 Hz, 1H), 6.93 (d, J = 8.1 Hz, 1H), 5.16 (q, J = 9.0 Hz, 2H), 3.48 (dd, J = 19.9, 6.2 Hz, 2H), 2.82 (q, J = 7.5 Hz, 2H), 1.32 (d, J = 20.0 Hz, 6H), 1.23 (t, J = 7.6 Hz, 3H). | 454.2 |
| 161 | 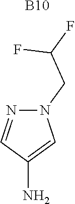 | 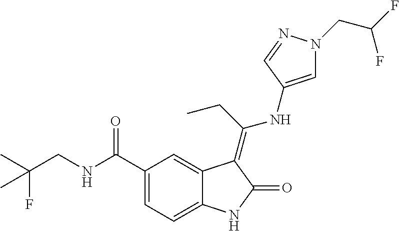 [SMILES: CC/C(Nc1cnn(CC(F)F)c1)=C1/C(=O)Nc2ccc(C(=O)NCC(C)(C)F)cc21] | K | 84 | 11.72 (s, 1H), 10.82 (s, 1H), 8.46 (t, J = 6.3 Hz, 1H), 8.04 (s, 1H), 7.79 (s, 1H), 7.68 (s, 1H), 7.60 (d, J = 8.1 Hz, 1H), 6.93 (d, J = 8.1 Hz, 1H), 6.40 (tt, J = 54.9, 3.8 Hz, 1H), 4.65 (dt, J = 15.0, 3.8 Hz, 2H), 3.50 (d, J = 4.0 Hz, 1H), 3.46 (br s, 1H), 2.83 (q, J = 7.5 Hz, 2H), 1.32 (d, J = 20.0 Hz, 6H), 1.24 (t, J = 7.5 Hz, 3H). | 436.2 |
| 162 | 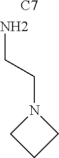 | 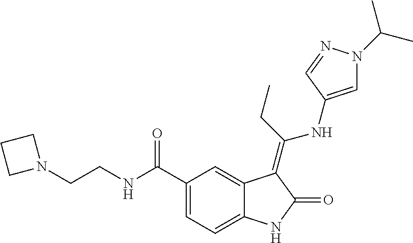 [SMILES: CC/C(Nc1cnn(C(C)C)c1)=C1/C(=O)Nc2ccc(C(=O)NCCN3CCC3)cc21] | J | 3 | 11.71 (s, 1H), 10.78 ( s, 1H), 8.22 (t, J = 5.6 Hz, 1H), 8.00 (s, 1H), 7.74 (s, 1H), 7.55 (s, 1H), 7.51 (d, J = 8.4 Hz, 1H), 6.89 (d, J = 8.4 Hz, 1H), 4.50 (m, 1H), 3.31 (m, 2H), 3.17 (m, 2H), 3.11 (t, J = 6.8 Hz, 4H), 2.81 (q, J = 7.6 Hz, 2H), 1.94 (m, 2H) , 1.42 (d, J = 6.4 Hz, 6H), 1.23 (t, J = 7.6 Hz, 3H). | 423.2 |
| 163 | 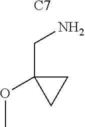 | 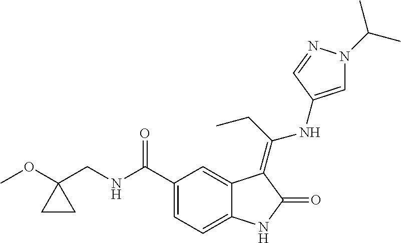 [SMILES: CC/C(Nc1cnn(C(C)C)c1)=C1/C(=O)Nc2ccc(C(=O)NCC3(OC)CC3)cc21] | I | 50 | 11.71 (s, 1H), 10.79 (s, 1H), 8.33 (t, J = 6.1 Hz, 1H), 8.00 (s, 1H), 7.77 (s, 1H), 7.56 (m, 2H), 6.92 (dd, J = 8.1, 1.4 Hz, 1H), 4.50 (m, 1H), 3.52 (d, J = 5.8 Hz, 2H), 3.26 (s, 3H), 2.82 (q, J = 7.5 Hz, 2H), 1.44 (d, J = 6.7 Hz, 6H), 1.24 (t, J = 7.6, 3H), 0.60-0.69 (m, 4H). | 424.2 |
| 164 | 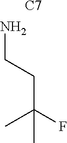 | 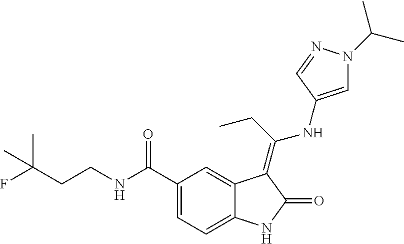 [SMILES: CC/C(Nc1cnn(C(C)C)c1)=C1/C(=O)Nc2ccc(C(=O)NCCC(C)(C)F)cc21] | I | 27 | 11.72 (s, 1H), 10.78 (s, 1H), 8.32 (t, J = 5.7 Hz, 1H), 8.01 (s, 1H), 7.75 (s, 1H), 7.56 (s, 1H), 7.54 (dd, J = 8.1, 1.5 Hz, 1H), 6.92 (d, J = 8.1 Hz, 1H), 4.51 (m, 1H), 3.37 (m, 2H), 2.82 (q, J = 7.5 Hz, 2H), 1.83-1.93 (m, 2H), 1.45 (d, J = 6.7 Hz, 6H), 1.37 (d, J = 20.0 Hz, 6H), 1.25 (t, J = 7.5 Hz, 3H). | 428.2 |
| 165 | 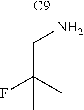 | 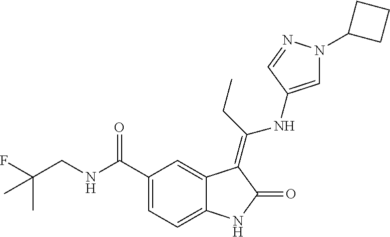 [SMILES: CC/C(Nc1cnn(C2CCC2)c1)=C1/C(=O)Nc2ccc(C(=O)NCC(C)(C)F)cc21] | J | 26 | 11.71 (s, 1H), 10.80 (s, 1H), 8.46 (t, J = 4.8 Hz, 1H), 8.06 (s, 1H), 7.78 (s, 1H), 7.59 (s, 1H), 7.57 (dd, J = 5.2, 1.2 Hz, 1H), 6.91 (d, J = 8.0 Hz, 1H), 4.84 (m, 1H), 3.47 (dd, J = 20.0, 6.4 Hz, 1H), 2.83 (q, J = 7.2 Hz, 2H), 2.35- 2.43 (m, 4H), 1.73- 1.82 (m, 2H), 1.31 (d, J = 21.6 Hz, 6H), 1.24 (t, J = 7.6 Hz, 3H). | 426.3 |
| 166 | 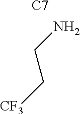 | 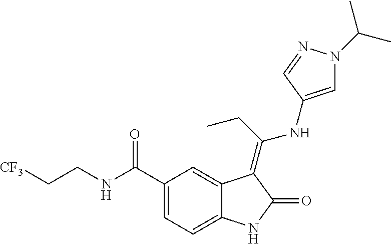 [SMILES: CC/C(Nc1cnn(C(C)C)c1)=C1/C(=O)Nc2ccc(C(=O)NCCC(F)(F)F)cc21] | J | 27 | 11.72 (s, 1H), 10.80 (s, 1H), 8.51 (t, J = 5.6 Hz, 1H), 8.00 (s, 1H), 7.75 (s, 1H), 7.55 (s, 1H), 7.53 (dd, J = 8.0 Hz, 1.2 Hz, 1H), 6.91 (d, J = 8.4 Hz, 1H), 4.50 (m, 1H), 3.46-3.51 (m, 2H), 2.82 (q, J = 7.6 Hz, 2H), 2.66 (m, 2H), 1.42 (d, J = 6.8 Hz, 6H), 1.24 (t, J = 7.6 Hz, 3H). | 436.2 |
| 167 | 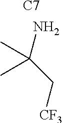 | 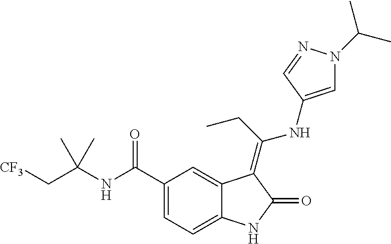 [SMILES: CC/C(Nc1cnn(C(C)C)c1)=C1/C(=O)Nc2ccc(C(=O)NC(C)(C)CC(F)(F)F)cc21] | J | 10 | 11.68 (s, 1H), 10.78 (s, 1H), 8.00 (s, 1H), 7.82 (s, 1H), 7.67 (s, 1H), 7.55 (s, 1H), 7.50 (dd, J = 8.0, 1.2 Hz, 1H), 6.90 (d, J = 8.0 Hz, 1H), 4.48 (m, 1H), 2.91 (q, J = 11.6 Hz, 2H), 2.78 (q, J = 7.2 Hz, 2H), 1.46 (s, 6H), 1.43 (d, J = 6.8 Hz, 6H), 1.24 (t, J = 7.2 Hz, 3H). | 464.3 |
| 168 | 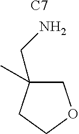 | 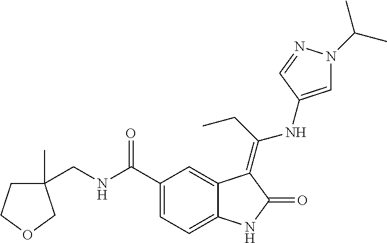 [SMILES: CC/C(Nc1cnn(C(C)C)c1)=C1/C(=O)Nc2ccc(C(=O)NCC3(C)CCOC3)cc21] | J | 25 | 11.70 (s, 1H), 10.79 (s, 1 H), 8.32 (t, J = 6.0 Hz, 1H), 8.00 (s, 1H), 7.74 (s, 1H), 7.56 (s, 1H), 7.53 (dd, J = 7.2, 0.8 Hz, 1H), 6.91 (d, J = 8.4 Hz, 1H), 4.49 (m, 1H), 3.69-3.80 (m, 2H), 3.61 (d, J = 8.4 Hz, 1H), 3.35 (m, 1H), 3.20-3.26 (m, 2H), 2.80 (q, J = 7.2 Hz, 2H), 1.88 (m, 1H), 1.53 (m, 1H), 1.42 (d, J = 6.8 Hz, 6H), 1.24 (t, J = | 438.2 |
| | | | | | 7.6 Hz, 3H), 1.04 (s, | |
| | | | | | 3H). | |
| 169 | 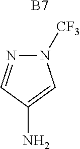 | 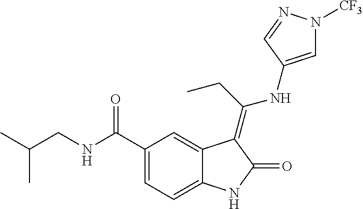 [SMILES: CC/C(Nc1cnn(C(F)(F)F)c1)=C1/C(=O)Nc2ccc(C(=O)NCC(C)C)cc21] | K (DBU) | 13 | 11.74 (s, 1H), 10.88 (s, 1H), 8.75 (s, 1H), 8.34 (t, J = 5.9 Hz, 1H), 8.18 (s, 1H), 7.79 (s, 1H), 7.60 (d, J = 8.1 Hz, 1H), 6.94 (d, J = 8.1 Hz, 1H), 3.08 (t, J = 6.4 Hz, 2H), 2.86 (q, J = 7.6 Hz, 2H), 1.85 (m, 1H), 1.25 (t, J = 7.6 Hz, 3H), 0.90 (d, J = 6.7 Hz, 6H). | 422.2 |
| 170 | 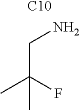 | 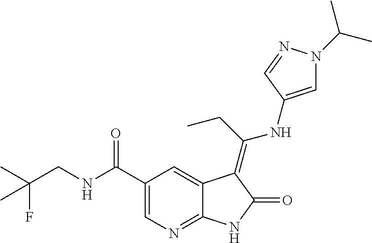 [SMILES: CC/C(Nc1cnn(C(C)C)c1)=C1/C(=O)Nc2ncc(C(=O)NCC(C)(C)F)cc21] | I | 9 | 11.63 (s, 1H), 11.37 (s, 1H), 8.64 (t, J = 6.3 Hz, 1H), 8.49 (s, 1H), 8.03 (s, 1H), 7.93 (s, 1H), 7.58 (s, 1H), 4.51 (m, 1H), 3.49 (dd, J = 20.2, 6.2 Hz, 2H), 2.80 (q, J = 7.6 Hz, 2H), 1.44 (d, J = 6.6 Hz, 6H), 1.33 (d, J = 20.0 Hz, 6H), 1.23 (t, J = 7.6 Hz, 3H). | 415.2 |
| 171 | 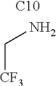 | 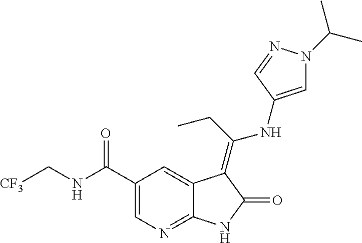 [SMILES: CC/C(Nc1cnn(C(C)C)c1)=C1/C(=O)Nc2ncc(C(=O)NCC(F)(F)F)cc21] | I | 5 | 11.64 (s, 1H), 11.44 (s, 1H), 9.08 (t, J = 6.3 Hz, 1H), 8.50 (d, J = 1.5 Hz, 1H), 8.04 (s, 1H), 7.94 (s, 1H), 7.59 (s, 1H), 4.51 (m, 1H), 4.01 (m, 2H), 2.80 (q, J = 7.5 Hz, 2H), 1.44 (d, J = 6.7 Hz, 6H), 1.24 (t, J = 7.5 Hz, 3H). | 423.2 |
| 172 | 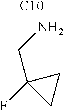 | 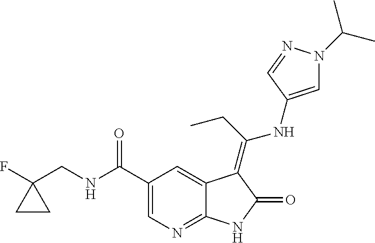 [SMILES: CC/C(Nc1cnn(C(C)C)c1)=C1/C(=O)Nc2ncc(C(=O)NCC3(F)CC3)cc21] | I | 4 | 11.63 (s, 1H), 11.38 (s, 1H), 8.82 (t, J = 5.9 Hz, 1H), 8.49 (s, 1H), 8.03 (s, 1H), 7.93 (s, 1H), 7.58 (s, 1H), 4.51 (m, 1H), 3.71 (dd, J = 20.9, 5.8 Hz, 2H), 2.80 (q, J = 7.5 Hz, 2H), 1.44 (d, J = 6.7, 6H), 1.24 (t, J = 7.6 Hz, 3H), 0.95- 1.06 (m, 2H), 0.79- 0.85 (m, 2H). | 413.2 |
| 173 | 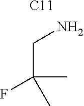 | 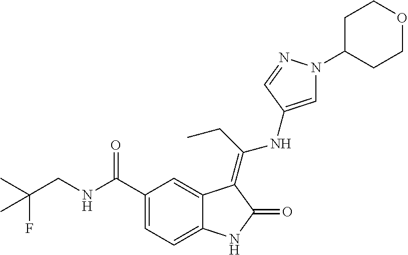 [SMILES: CC/C(Nc1cnn(C2CCOCC2)c1)=C1/C(=O)Nc2ccc(C(=O)NCC(C)(C)F)cc21] | J | 41 | 11.71 (s, 1H), 10.80 (s, 1H), 8.46 (t, J = 4.8 Hz, 1H), 8.06 (s, 1H), 7.78 (s, 1H), 7.59 (s, 1H), 7.57 (m, 1H), 6.91 (d, J = 8.4 Hz, 1H), 4.40 (m, 1H), 3.95 (m, 2H), 3.44-3.50 (m, 4H), 2.81 (q, J = 7.6 Hz, 2H), 1.93-1.99 (m, 4H), 1.33 (d, J = 21.6 Hz, 6H), 1.24 (t, J = 7.6 Hz, 3H). | 456.2 |
| 174 | 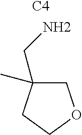 | 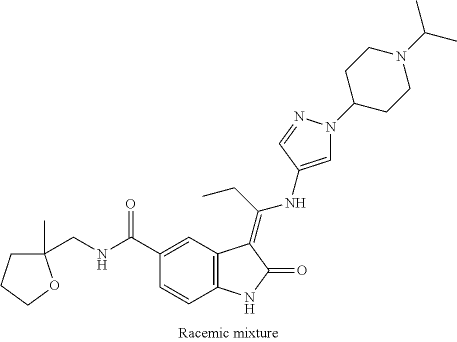 [SMILES: CC/C(Nc1cnn(C2CCN(C(C)C)CC2)c1)=C1/C(=O)Nc2ccc(C(=O)NCC3(C)CCCO3)cc21] | J | 17 | 11.70 (s, 1H), 10.78 (s, 1H), 8.24 (t, J = 6.0 Hz, 1H), 8.02 (s, 1H), 7.75 (s, 1H), 7.54 (m, 2H), 6.90 (d, J = 8.4 Hz, 1H), 4.11 (m, 1H), 3.70-3.79 (m, 2H), 3.37 (m, 1H), 3.25 (m, 1H), 2.81-2.88 (m, 2H), 2.66-2.80 (m, 3H), 2.28 (m, 2H), 2.01 (m, 2H), 1.81-1.90 (m, 5H), 1.53 (m, 1H), 1.25 (t, J = 7.6 Hz, 3H), 1.21 (s, 3H), 0.98 (d, J = 6.4 Hz, 6H). | 521.3 |
| 175 | 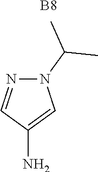 | 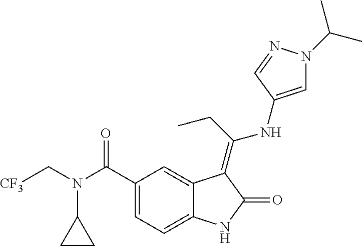 [SMILES: CC/C(Nc1cnn(C(C)C)c1)=C1/C(=O)Nc2ccc(C(=O)N(CC(F)(F)F)C3CC3)cc21] | K | 18 | 11.68 (s, 1H), 10.80 (s, 1H), 7.99 (s, 1H), 7.54 (s, 1H), 7.37 (br s, 1H), 7.22 (br d, J = 8.0 Hz, 1H), 6.92 (d, J = 8.0 Hz, 1H), 4.49 (m, 1H), 4.31 (q, J = 9.6 Hz, 2H), 2.94 (m, 1H), 2.70 (q, J = 7.6 Hz, 2H), 1.42 (d, J = 6.8 Hz, 6H), 1.21 (t, J = 7.6 Hz, 3H), 0.61 (m, 2H), 0.49 (m, 2H). | 462.2 |
| 176 | 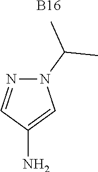 | 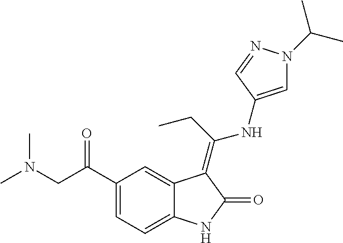 [SMILES: CC/C(Nc1cnn(C(C)C)c1)=C1/C(=O)Nc2ccc(C(=O)CN(C)C)cc21] | K | 15 | 11.65 (s, 1H), 10.94 (br s, 1H), 8.01 (s, 1H), 7.98 (br s, 1H), 7.75 (dd, J = 8.0, 1.2 Hz, 1H), 7.56 (s, 1H), 6.95 (d, J = 8.0 Hz, 1H), 4.50 (m, 1H), 3.66 (s, 2H), 2.79 (q, J = 7.2 Hz, 2H), 2.23 (s, 6H), 1.42 (d, J = 6.8 Hz, 6H), 1.24 (t, J = 5.6 Hz, 3H). | 382.2 |
| 177 | 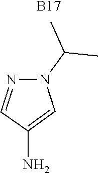 | 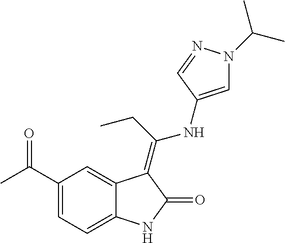 [SMILES: CC/C(Nc1cnn(C(C)C)c1)=C1/C(=O)Nc2ccc(C(C)=O)cc21] | K and piperidine | 73 | 11.68 (s, 1H), 10.95 (s, 1H), 8.01 (s, 1H), 7.83 (d, J = 1.6 Hz, 1H), 7.71 (dd, J = 8.2, 1.6 Hz, 1H), 7.56 (d, J = 0.8 Hz, 1H), 6.98 (d, J = 8.2 Hz, 1H), 4.50 (m, 1H), 2.79 (q, J = 7.6 Hz, 2H), 2.55 (s, 3H), 1.44 (d, J = 6.7 Hz, 6H), 1.27 (t, J = 7.6 Hz, 3H). | 339.2 |
| 178 | 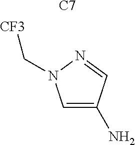 | 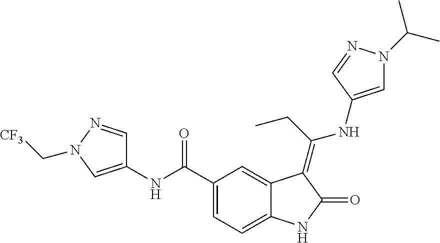 [SMILES: CC/C(Nc1cnn(C(C)C)c1)=C1/C(=O)Nc2ccc(C(=O)Nc3cnn(CC(F)(F)F)c3)cc21] | J | 10 | 11.73 (s, 1H), 10.86 (s, 1H), 10.33 (s, 1H), 8.20 (s, 1H), 8.15 (s, 1H), 7.84 (s, 1H), 7.72 (s, 1H), 7.67 (br d, J = 4.8 Hz, 1H), 7.56 (s, 1H), 6.98 (d, J = 8.0 Hz, 1H), 5.11 (q, J = 8.8 Hz, 2H), 4.51 (m, 1H), 2.83 (d, J = 7.6 Hz, 2H), 1.43 (d, J = 6.4 Hz, 6H), 1.28 (t, J = 7.6 Hz, 3H). | 488.4 |
| 179 | 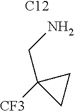 | 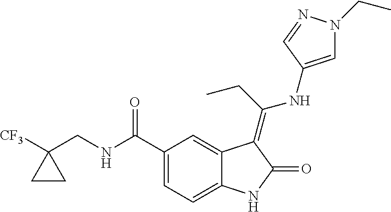 [SMILES: CC/C(Nc1cnn(CC)c1)=C1/C(=O)Nc2ccc(C(=O)NCC3(C(F)(F)F)CC3)cc21] | J | 53 | 11.70 (s, 1H), 10.87 (s, 1H), 8.42 (t, J = 6.0 Hz, 1H), 7.98 (s, 1H), 7.73 (s, 1H), 7.55 (s, 1H), 7.51 (br d, J = 8.0 Hz, 1H), 6.92 (d, J = 8.0 Hz, 1H), 4.15 (q, J = 7.2 Hz, 1H), 3.59 (d, J = 6.0 Hz, 2H), 2.82 (q, J = 7.6 Hz, 2H), 1.39 (t, J = 7.2 Hz, 3H), 1.24 (t, J = 7.6 Hz, 3H), 0.89 (m, 4H). | 448.2 |
| 180 | 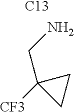 | 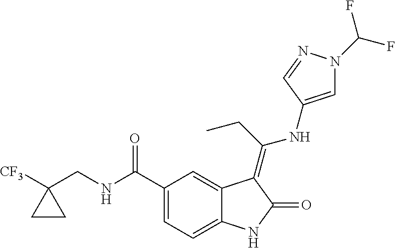 [SMILES: CC/C(Nc1cnn(C(F)F)c1)=C1/C(=O)Nc2ccc(C(=O)NCC3(C(F)(F)F)CC3)cc21] | J | 37 | 11.73 (s, 1H), 10.87 (s, 1H), 8.50 (s, 1H), 8.44 (t, J = 6.0 Hz, 1H), 7.98 (s, 1H), 7.82 (t, J = 59.2 Hz, 1H), 776 (s, 1H), 7.54 (d, J = 8.4 Hz, 1H), 6.93 (d, J = 8.0 Hz, 1H), 3.59 (d, J = 6.0 Hz, 2H), 2.84 (q, J = 7.6 Hz, 2H), 1.24 (t, J = 7.2 Hz, 3H), 0.85- 0.94 (m, 4H). | 470.2 |
| 181 | 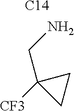 | 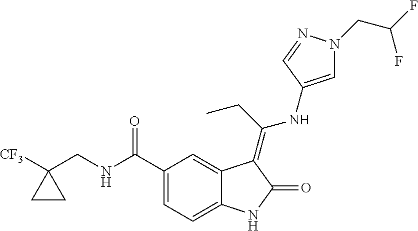 [SMILES: CC/C(Nc1cnn(CC(F)F)c1)=C1/C(=O)Nc2ccc(C(=O)NCC3(C(F)(F)F)CC3)cc21] | J | 50 | 11.71 (s, 1H), 10.83 (s, 1H), 8.42 (t, J = 6.4 Hz, 1H), 8.03 (s, 1H), 7.74 (s, 1H), 7.68 (s, 1H), 7.52 (d, J = 8.4 Hz, 1H), 6.92 (d, J = 8.0 Hz, 1H), 6.40 (tt, J = 54.4, 4.0 Hz, 1H), 4.65 (dt, J = 15.2, 3.6 Hz, 2H), 3.59 (d, J = 6.0 Hz, 2H), 2.82 (q, J = 7.6 Hz, 2H), 1.23 (t, J = 7.6 Hz, 3H), 0.87- 0.94 (m, 4H). | 484.3 |
| 182 | 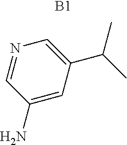 | 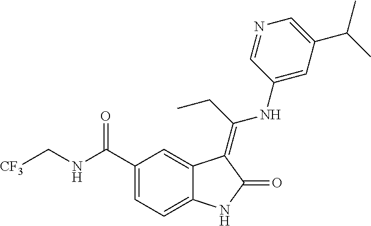 [SMILES: CC/C(Nc1cncc(C(C)C)c1)=C1/C(=O)Nc2ccc(C(=O)NCC(F)(F)F)cc21] | K | 44 | 12.11 (S, 1H), 10.98 (s, 1H), 8.93 (s, J = 6.0 Hz, 1H), 8.45 (d, J = 2.0 Hz, 1H), 8.42 (d, J = 2.4 Hz, 1H), 7.83 (s, 1H), 7.69 (br s, 1H), 7.64 (d, J = 0.8 Hz, 1H), 6.97 (d, J = 8.0 Hz, 1H), 4.03-4.12 (m, 2H), 3.01 (m, 1H), 2.84 (q, J = 7.2 Hz, 2H), 1.26 (d, J = 6.8 Hz, 6H), 1.24 (t, J = 7.6 Hz, 3H). | 433.2 |
| 183 | C7 | 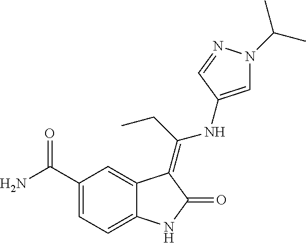 [SMILES: CC/C(Nc1cnn(C(C)C)c1)=C1/C(=O)Nc2ccc(C(N)=O)cc21] | J | 1 | 11.71 (s, 1H), 10.78 (s, 1H), 8.00 (s, 1H), 7.85 (br s, 1H), 7.78 (s, 1H), 7.57 (d, J = 8.4 Hz, 1H), 7.55 (s, 1H), 7.11 (br, s, 1H), 6.89 (d, J = 8.0 Hz, 1H), 4.49 (m, 1H), 2.81 (q, J = 7.2 Hz, 2H), 1.42 (d, J = 6.4 Hz, 6H), 1.23 (t, J = 7.6 Hz, 3H). | 340.1 |

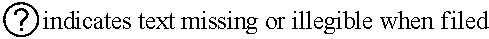

### Biochemical Assay for ULK1 (SEQ. ID NO: 1)

Activity of ULK1 kinase was determined spectroscopically using a coupled pyruvate kinase/lactate dehydrogenase assay that continuously monitors the ATP hydrolysis-dependent oxidation of NADH (e.g., Schindler et al. Science (2000) 289: 1938-1942). Assays were conducted in 384-well plates (100 μL final volume) using 0.1 nM ULK1 (from Beryllium), 0.075 mM peptide substrate (YANWLAASIYLDGKKK), 1.5 units pyruvate kinase, 2.1 units lactate dehydrogenase, 1 mM phosphoenol pyruvate, 0.28 mM NADH and 1 mM ATP in assay buffer (100 mM Tris, pH 7.5, 15 mM MgCl₂, 0.5 mM DTT, 0.004% (w/v) BSA, and 0.004% Triton X-100). Inhibition of ULK1 was measured by adding serial diluted test compound (final assay concentration of 1% DMSO). A decrease in absorption at 340 nm was monitored continuously for 6 h at 30° C. on a multi-mode microplate reader (BioTek). The reaction rate was calculated using the 2-3 h time frame. The reaction rate at each concentration of compound was converted to percent inhibition using controls (i.e., reaction with no test compound and reaction with a known inhibitor) and IC₅₀ values were calculated using software routines in Prism (GraphPad software).

ULK1 protein sequence
(residues 1-283; SEQ. ID NO: 1)
MEPGRGGTETVGKFEFSRKDLIGHGAFAVVFKGRHRAAHDLEVAVKCIN
KKNLAKSQTLLGKEIKILKELKHENIVALYDFQEMANSVYLVMEYCNGG
DLADYLHAMRTLSEDTIRLFLQQIAGAMRLLHSKGIIHRDLKPQNILLS
NPAGRRANPNSIRVKIADFGFARYLQSNMMAATLCGSPMYMAPEVIMSQ
HYDGKADLWSIGTIVYQCLTGKAPFQASSPQDLRLFYEKNKTLVPTIPR
ETSAPLRQLLLALLQRNHKDRMDFDEFFHHPFLDASPS

### Biochemical Assay for ULK2 (SEQ. ID NO: 2)

Activity of ULK2 kinase was determined spectroscopically using a coupled pyruvate kinase/lactate dehydrogenase assay that continuously monitors the ATP hydrolysis-dependent oxidation of NADH (e.g., Schindler et al. Science (2000) 289: 1938-1942). Assays were conducted in 384-well plates (100 μL final volume) using 1 nM ULK2 (Signal Chem U02-11G), 200 μM ULKtide, 1.5 units pyruvate kinase, 2.1 units lactate dehydrogenase, 1 mM phosphoenol pyruvate, 0.28 mM NADH and 1 mM ATP in assay buffer (100 mM Tris, pH 7.5, 15 mM MgCl₂, 0.5 mM DTT, 0.1% octyl-glucoside, 0.002% (w/v) BSA, and 0.002% Triton X-100). Inhibition of ULK2 was measured by adding serial diluted test compound (final assay concentration of 1% DMSO). A decrease in absorption at 340 nm was monitored continuously for 6 h at 30° C. on a multi-mode microplate reader (BioTek). The reaction rate was calculated using the 2-3 h time frame. The reaction rate at each concentration of compound was converted to percent inhibition using controls (i.e., reaction with no test compound and reaction with a known inhibitor) and IC₅₀ values were calculated by fitting a four-parameter sigmoidal curve to the data using Prism (GraphPad software).

ULK2 protein sequence
(residues 1-306 with N-terminal GST and His tag; SEQ. ID NO: 2)
MSPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPKSDLEVLFQGPEFMEVVGDFEYSKRDLV
GHGAFAVVFRGRHRQKTDWEVAIKSINKKNLSKSQILLGKEIKILKELQ
HENIVALYDVQELPNSVFLVMEYCNGGDLADYLQAKGTLSEDTIRVFLH
QIAAAMRILHSKGIIHRDLKPQNILLSYANRRKSSVSGIRIKIADFGFA
RYLHSNMMAATLCGSPMYMAPEVIMSQHYDAKADLWSIGTVIYQCLVGK
PPFQANSPQDLRMFYEKNRSLMPSIPRETSPYLANLLLGLLQRNQKDRM
DFEAFFSHPFLEQGPVKKSCPVPVPMYSGSVSGSSCGSSPSCRFASHHH
HHH

TABLE 1
Inhibition of biochemical activity of ULK1
and ULK2 kinases by exemplary compounds.

| Example | | |
|---|---|---|
| (Compound) | | |
| Number | ULK1 | ULK2 |
| 1 | + | ++ |
| 2 | +++ | |
| 3 | ++ | +++ |
| 4 | + | ++ |
| 5 | ++ | +++ |
| 6 | ++ | +++ |
| 7 | + | ++ |
| 8 | + | ++ |
| 9 | ++ | |
| 10 | +++ | |
| 11 | ++ | |
| 12 | +++ | |
| 13 | +++ | |
| 14 | +++ | |
| 15 | + | |
| 16 | + | ++ |
| 17 | ++ | +++ |
| 18 | ++ | |
| 19 | ++ | ++ |
| 20 | ++++ | |
| 21 | ++ | ++ |
| 22 | ++ | |
| 23 | ++ | |
| 24 | + | |
| 25 | +++ | ++++ |
| 26 | + | ++ |
| 27 | ++ | +++ |
| 28 | ++ | ++ |
| 29 | + | ++ |
| 30 | + | + |
| 31 | + | + |
| 32 | + | |
| 33 | + | |
| 34 | + | |
| 35 | + | |
| 36 | + | |
| 37 | + | ++ |
| 38 | + | |
| 39 | + | |
| 40 | + | |
| 41 | + | |
| 42 | + | ++ |
| 43 | + | |
| 44 | + | + |
| 45 | + | |
| 46 | ++ | |
| 47 | + | ++ |
| 48 | ++ | ++ |
| 49 | +++ | |
| 50 | +++ | |
| 51 | + | ++ |
| 52 | + | ++ |
| 53 | + | ++ |
| 54 | + | ++ |
| 55 | ++ | +++ |
| 56 | ++ | +++ |
| 57 | +++ | |
| 58 | ++ | +++ |
| 59 | +++ | |
| 60 | + | ++ |
| 61 | + | ++ |
| 62 | + | ++ |
| 63 | ++ | ++ |
| 64 | ++ | ++ |
| 65 | + | ++ |
| 66 | + | ++ |
| 67 | + | ++ |
| 68 | + | ++ |
| 69 | + | ++ |
| 70 | + | ++ |
| 71 | + | ++ |
| 72 | + | + |
| 73 | +++ | +++ |
| 74 | ++ | +++ |
| 75 | + | + |
| 76 | +++ | ++++ |
| 77 | + | ++ |
| 78 | ++ | ++ |
| 79 | ++ | ++ |
| 80 | + | + |
| 81 | ++++ | |
| 82 | + | + |
| 83 | + | ++ |
| 84 | + | + |
| 85 | ++ | ++ |
| 86 | + | ++ |
| 87 | ++ | ++ |
| 88 | + | ++ |
| 89 | ++ | +++ |
| 90 | + | ++ |
| 91 | + | ++ |
| 92 | +++ | +++ |
| 93 | + | ++ |
| 94 | ++ | ++ |
| 95 | + | ++ |
| 96 | + | ++ |
| 97 | +++ | ++ |
| 98 | + | +++ |
| 99 | +++ | ++++ |
| 100 | +++ | +++ |
| 101 | + | ++ |
| 102 | ++ | ++ |
| 103 | + | ++ |
| 104 | + | ++ |
| 105 | + | ++ |
| 106 | + | ++ |
| 107 | + | ++ |
| 108 | ++ | ++ |
| 109 | +++ | ++++ |
| 110 | ++ | +++ |
| 111 | +++ | +++ |
| 112 | ++ | ++ |
| 113 | ++ | ++ |
| 114 | ++ | ++ |
| 115 | ++++ | ++++ |
| 116 | ++ | ++ |
| 117 | + | + |
| 118 | + | ++ |
| 119 | + | ++ |
| 120 | ++ | +++ |
| 121 | ++++ | ++++ |
| 122 | ++ | ++ |
| 123 | +++ | +++ |
| 124 | +++ | ++++ |
| 125 | ++ | ++ |
| 126 | ++ | +++ |
| 127 | ++ | ++ |
| 128 | ++ | +++ |
| 129 | ++ | +++ |
| 130 | + | ++ |
| 131 | +++ | +++ |
| 132 | +++ | ++++ |
| 133 | +++ | ++++ |
| 134 | + | ++ |
| 135 | + | ++ |
| 136 | + | ++ |
| 137 | + | ++ |
| 138 | ++ | +++ |
| 139 | +++ | |
| 140 | + | ++ |
| 141 | +++ | +++ |
| 142 | ++ | ++ |
| 143 | +++ | ++++ |
| 144 | + | ++ |
| 145 | ++ | +++ |
| 146 | +++ | +++ |
| 147 | ++ | +++ |
| 148 | ++ | +++ |
| 149 | ++ | ++ |
| 150 | +++ | ++++ |
| 151 | ++ | +++ |
| 152 | + | + |
| 153 | + | + |
| 154 | + | ++ |
| 155 | ++ | +++ |
| 156 | + | ++ |
| 157 | ++ | +++ |
| 158 | ++ | +++ |
| 159 | ++ | ++ |
| 160 | + | ++ |
| 161 | + | ++ |
| 162 | ++++ | ++++ |
| 163 | ++ | +++ |
| 164 | + | ++ |
| 165 | + | + |
| 166 | + | + |
| 167 | +++ | +++ |
| 168 | ++ | ++ |
| 169 | ++ | ++ |
| 170 | +++ | +++ |
| 171 | +++ | +++ |
| 172 | ++ | +++ |
| 173 | + | + |
| 174 | + | ++ |
| 175 | + | +++ |
| 176 | ++++ | ++++ |
| 177 | +++ | +++ |
| 178 | ++++ | ++++ |
| 179 | + | |
| 180 | + | |
| 181 | + | |
| 182 | ++++ | ++++ |
| 183 | ++ | ++ |

For Table 1, “+” refers to an IC₅₀ less than or equal to 10 nM; “++” refers to an IC₅₀ greater than 10 nM and less than or equal to 100 nM; “+++” refers to an IC₅₀ greater than 100 nM and less than or equal to 500 nM; and “++++” refers to an IC₅₀ greater than 500 nM.

Cellular Inhibition of ULK Kinase Substrate ATG13 Protein pATG13 Levels of Mutant KRas A549 Cells after Treatment with ULK Inhibitors in Combination with Trametinib

A549 (KRAS mutant) human lung cancer cells (6,000 cells/well) were added to a 384-well tissue-culture treated plate in 50 μL of pre-warmed DMEM medium supplemented with 10% characterized fetal bovine serum (Invitrogen, Carlsbad, CA), 100 units/mL penicillin G, and 100 μg/mL streptomycin and allowed to grow overnight at 37° C., 5% CO₂, and 95% humidity. The following day, 10 μL of media containing trametinib or DMSO as a control was added to wells. The final concentration of trametinib in wells was 250 nM. A dose response of a test compound (0.6 μL per well) was added. DMSO (0.6 μL) was added to control wells. The plate was briefly shaken to mix wells and then incubated at 37° C. overnight. The next day, the media was aspirated and cells were washed with Dulbecco's Phosphate Buffered Saline (Gibco). Cells were lysed using MPER lysis buffer (Pierce, Rockford, IL) containing Halt Phosphatase and Protease Inhibitors (Pierce, Rockford, IL) and Phosphatase inhibitor cocktail 2 (Sigma, St. Louis, MO) at 4° C. for 10 min with shaking.

Cellular levels of phospho-Serine 318 ATG13 (pATG13) were measured via an ELISA method. Total ATG13 Antibody (Cell Signaling Cat #13273) was used to coat the wells. The plate was incubated at 4° C. overnight and washed with ELISA wash buffer (Biolegend Cat #421601). The wells were then blocked with assay diluent (Biolegend Cat #421203) for 1 h at rt. Plate wells were washed with ELISA wash buffer. Cell lysate was added to wells and incubated at rt for 2 h. Plate wells were washed with ELISA wash buffer. Biotinylated pS318-ATG13 antibody (Rockland Immunochemicals Cat #600-401-C49) was diluted in assay diluent and added to each well and incubated at rt for 1 h. Plate wells were washed with ELISA wash buffer. Streptavidin linked to horseradish peroxidase (Thermo Fisher Cat #21140) was diluted in assay diluent and added to each well and incubated at rt for 1 h. Plate wells were washed with ELISA wash buffer. High sensitivity TMB substrate (Biolegend Cat #421101) was added to each well and incubated at rt for 20 min. The reaction was stopped with 2N Sulfuric Acid. The plate was analyzed at on a plate reader measuring absorbance at 450 nm and 540 nm (background). Signal was calculated by first subtracting the background absorbance at 540 nm from the absorbance at 450 nm for each well. Next, the background corrected absorbance at 450 nm from blank wells was subtracted from test wells. Data was compared to control wells to determine % ATG13 phosphorylation. GraphPad Prism was used to calculate IC₅₀ values.

pATG13 Levels of Mutant KRas MiaPaCa-2 Cells after Treatment with ULK Inhibitors in Combination with Trametinib

MiaPaCa-2 human pancreatic cancer cells (10000 cells/well) were added to a 384-well tissue-culture treated plate in 50 μL of pre-warmed DMEM medium supplemented with 10% characterized fetal bovine serum (Invitrogen, Carlsbad, CA), 100 units/mL penicillin G, 100 μg/mL streptomycin, and 2.5% Horse Serum and allowed to grow overnight at 37° C., 5% CO₂, and 95% humidity. The following day, 10 μL of media containing trametinib or DMSO as a control was added to wells. The final concentration of trametinib in wells was 250 nM. A dose response of a test compound (0.6 μL per well) was added. DMSO (0.6 μL) was added to control wells. The plate was briefly shaken to mix wells and then incubated at 37° C. overnight. The next day, the media was aspirated and cells were washed with Dulbecco's Phosphate Buffered Saline (Gibco). Cells were lysed using MPER lysis buffer (Pierce, Rockford, IL) containing Halt Phosphatase and Protease Inhibitors (Pierce, Rockford, IL) and Phosphatase inhibitor cocktail 2 (Sigma, St. Louis, MO) at 4° C. for 10 minutes with shaking.

Cellular levels of phospho-Serine 318 ATG13 (pATG13) were measured via an ELISA method. Total ATG13 Antibody (Cell Signaling Cat #13273) was used to coat the wells. The plate was incubated at 4° C. overnight and washed with ELISA wash buffer (Biolegend Cat #421601). The wells were then blocked with assay diluent (Biolegend Cat #421203) for 1 h at rt. Plate wells were washed with ELISA wash buffer. Cell lysate was added to wells and incubated at rt for 2 h. Plate wells were washed with ELISA wash buffer. Biotinylated pS318-ATG13 antibody (Rockland Immunochemicals Cat #600-401-C49) was diluted in assay diluent and added to each well and incubated at rt for 1 h. Plate wells were washed with ELISA wash buffer. Streptavidin linked to horseradish peroxidase (Thermo Fisher Cat #21140) was diluted in assay diluent and added to each well and incubated at rt for 1 h. Plate wells were washed with ELISA wash buffer. High sensitivity TMB substrate (Biolegend Cat #421101) was added to each well and incubated at rt for 20 min. The reaction was stopped with 2.0N sulfuric acid. The plate was analyzed at on a plate reader measuring absorbance at 450 nm and 540 nm (background). Signal was calculated by first subtracting the background absorbance at 540 nm from the absorbance at 450 nm for each well. Next, the background corrected absorbance at 450 nm from blank wells was subtracted from test wells. Data was compared to control wells to determine % ATG13 phosphorylation. GraphPad Prism was used to calculate IC₅₀ values.

pATG13 Levels of Mutant KRas HCT-116 Cells after Treatment with ULK Inhibitors in Combination with Trametinib

HCT-116 human colon cancer cells (10000 cells/well) were added to a 384-well tissue-culture treated plate in 50 μL of pre-warmed DMEM medium supplemented with 10% characterized fetal bovine serum (Invitrogen, Carlsbad, CA), 100 units/mL penicillin G, and 100 μg/mL streptomycin and allowed to grow overnight at 37° C., 5% CO₂, and 95% humidity. The following day, 10 μL of media containing trametinib or DMSO as a control was added to wells. The final concentration of trametinib in wells was 250 nM. A dose response of a test compound (0.6 μL per well) was added. DMSO (0.6 μL) was added to control wells. The plate was briefly shaken to mix wells and then incubated at 37° C. overnight. The next day, the media was aspirated and cells were washed with Dulbecco's Phosphate Buffered Saline (Gibco). Cells were lysed using MPER lysis buffer (Pierce, Rockford, IL) containing Halt Phosphatase and Protease Inhibitors (Pierce, Rockford, IL) and Phosphatase inhibitor cocktail 2 (Sigma, St. Louis, MO) at 4° C. for 10 minutes with shaking.

Cellular levels of phospho-Serine 318 ATG13 (pATG13) were measured via an ELISA method. Total ATG13 Antibody (Cell Signaling Cat #13273) was used to coat the wells. The plate was incubated at 4° C. overnight and washed with ELISA wash buffer (Biolegend Cat #421601). The wells were then blocked with assay diluent (Biolegend Cat #421203) for 1 h at rt. Plate wells were washed with ELISA wash buffer. Cell lysate was added to wells and incubated at rt for 2 h. Plate wells were washed with ELISA wash buffer. Biotinylated pS318-ATG13 antibody (Rockland Immunochemicals Cat #600-401-C49) was diluted in assay diluent and added to each well and incubated at rt for 1 h. Plate wells were washed with ELISA wash buffer. Streptavidin linked to horseradish peroxidase (Thermo Fisher Cat #21140) was diluted in assay diluent and added to each well and incubated at rt for 1 h. Plate wells were washed with ELISA wash buffer. High sensitivity TMB substrate (Biolegend Cat #421101) was added to each well and incubated at rt for 20 minutes. The reaction was stopped with 2.0N sulfuric acid. The plate was analyzed at on a plate reader measuring absorbance at 450 nm and 540 nm (background). Signal was calculated by first subtracting the background absorbance at 540 nm from the absorbance at 450 nm for each well. Next, the background corrected absorbance at 450 nm from blank wells was subtracted from test wells. Data was compared to control wells to determine % ATG13 phosphorylation. GraphPad Prism was used to calculate IC₅₀ values.

pATG13 Levels of Mutant HRas T24 Cells after Treatment with ULK Inhibitors in Combination with Trametinib

T24 human urinary bladder cancer cells (25000 cells/well) were added to a 96-well tissue-culture treated plate in 100 μL of pre-warmed DMEM medium supplemented with 10% characterized fetal bovine serum (Invitrogen, Carlsbad, CA), 100 units/mL penicillin G, and 100 μg/mL streptomycin and allowed to grow overnight at 37° C., 5% CO₂, and 95% humidity. The following day, 100 μL of media containing trametinib or DMSO as a control was added to wells. The final concentration of trametinib in wells was 250 nM. A dose response of a test compound (0.5 μL per well) was added. DMSO (0.5 μL) was added to control wells. The plate was briefly shaken to mix wells and then incubated at 37° C. overnight. The next day, the media was aspirated and cells were washed with Dulbecco's Phosphate Buffered Saline (Gibco). Cells were lysed using MPER lysis buffer (Pierce, Rockford, IL) containing Halt Phosphatase and Protease Inhibitors (Pierce, Rockford, IL) and Phosphatase inhibitor cocktail 2 (Sigma, St. Louis, MO) at 4° C. for 10 min with shaking.

Cellular levels of phospho-Serine 318 ATG13 (pATG13) were measured via an ELISA method. Total ATG13 Antibody (Cell Signaling Cat #13273) was used to coat the wells. The plate was incubated at 4° C. overnight and washed with ELISA wash buffer (Biolegend Cat #421601). The wells were then blocked with assay diluent (Biolegend Cat #421203) for 1 h at rt. Plate wells were washed with ELISA wash buffer. Cell lysate was added to wells and incubated at rt for 2 h. Plate wells were washed with ELISA wash buffer. Biotinylated pS318-ATG13 antibody (Rockland Immunochemicals Cat #600-401-C49) was diluted in assay diluent and added to each well and incubated at rt for 1 h. Plate wells were washed with ELISA wash buffer. Streptavidin linked to horseradish peroxidase (Thermo Fisher Cat #21140) was diluted in assay diluent and added to each well and incubated at rt for 1 h. Plate wells were washed with ELISA wash buffer. High sensitivity TMB substrate (Biolegend Cat #421101) was added to each well and incubated at rt for 20 min. The reaction was stopped with 2.0N sulfuric acid. The plate was analyzed at on a plate reader measuring absorbance at 450 nm and 540 nm (background). Signal was calculated by first subtracting the background absorbance at 540 nm from the absorbance at 450 nm for each well. Next, the background corrected absorbance at 450 nm from blank wells was subtracted from test wells. Data was compared to control wells to determine % ATG13 phosphorylation. GraphPad Prism was used to calculate IC₅₀ values.

TABLE 2
Inhibition of ULK kinase in mutant Ras.

| Example | A549 | MiaPaca-2 | HCT-116 | T24 |
|---|---|---|---|---|
| (Compound) | pATG13 | pATG13 | pATG13 | pATG13 |
| No. | ELISA | ELISA | ELISA | ELISA |
| 1 | ++ | + | + | + |
| 2 | ++++ | ++ | | |
| 3 | ++ | ++ | | |
| 4 | ++ | + | + | |
| 5 | ++++ | ++ | | |
| 6 | ++ | | | |
| 7 | + | + | + | + |
| 8 | + | + | + | + |
| 9 | +++ | ++ | + | ++ |
| 11 | +++ | + | | |
| 15 | + | + | | |
| 16 | + | + | + | + |
| 21 | ++++ | + | | |
| 24 | +++ | + | | |
| 26 | ++++ | + | + | |
| 27 | ++ | + | | |
| 28 | ++++ | + | | |
| 29 | +++ | + | | |
| 30 | ++ | + | | |
| 31 | + | + | | |
| 32 | + | + | | |
| 33 | + | + | | |
| 34 | + | + | | |
| 35 | +++ | + | | |
| 36 | ++ | + | | |
| 37 | + | + | | |
| 38 | + | + | | |
| 39 | + | + | | |
| 40 | + | + | | |
| 41 | + | + | | |
| 42 | + | + | | |
| 43 | + | + | | |
| 44 | + | + | | |
| 46 | ++ | + | | |
| 47 | + | + | + | |
| 48 | + | + | + | + |
| 51 | ++++ | + | | |
| 52 | ++++ | + | + | |
| 53 | ++++ | + | + | |
| 54 | +++ | + | + | |
| 55 | ++ | + | | |
| 58 | ++ | + | + | |
| 60 | + | + | + | |
| 61 | + | + | + | |
| 62 | ++ | + | | |
| 63 | + | + | | |
| 64 | + | + | | |
| 65 | + | + | | |
| 66 | + | + | | |
| 67 | + | + | | |
| 68 | + | + | | |
| 69 | + | + | | |
| 70 | + | + | | |
| 71 | + | + | | |
| 72 | | + | | |
| 73 | | ++ | | |
| 74 | ++ | + | | |
| 75 | ++ | + | + | |
| 76 | | ++ | | |
| 77 | ++ | + | + | |
| 78 | +++ | ++ | | |
| 79 | +++ | + | | |
| 80 | + | + | | |
| 82 | +++ | + | ++++ | |
| 83 | + | + | | |
| 84 | ++++ | + | | |
| 85 | +++ | ++ | | |
| 86 | +++ | + | + | |
| 87 | +++ | + | | |
| 88 | ++ | + | | |
| 89 | ++++ | ++ | | |
| 90 | +++ | + | | |
| 91 | ++ | + | | |
| 92 | + | + | | |
| 93 | + | + | + | |
| 94 | ++ | + | | |
| 95 | + | + | | |
| 98 | +++ | + | + | + |
| 100 | +++ | ++ | | |
| 101 | + | + | | |
| 102 | ++ | + | | |
| 103 | +++ | + | | |
| 104 | + | + | | |
| 105 | + | + | | |
| 106 | ++ | + | | |
| 107 | + | + | + | |
| 108 | ++++ | ++ | | |
| 112 | + | + | | |
| 113 | ++ | + | + | |
| 114 | ++ | + | + | |
| 116 | + | + | | |
| 117 | + | + | + | |
| 118 | +++ | + | | |
| 119 | + | + | | |
| 122 | ++++ | ++ | | |
| 123 | ++ | + | ++ | |
| 124 | ++++ | +++ | | |
| 125 | +++ | ++ | | |
| 126 | +++ | ++ | | |
| 127 | ++++ | ++ | | |
| 128 | +++ | +++ | | |
| 130 | + | + | + | |
| 134 | + | + | | |
| 135 | + | + | + | |
| 136 | ++ | + | | |
| 137 | +++ | ++ | | |
| 138 | ++++ | +++ | | |
| 140 | + | + | + | |
| 142 | +++ | ++ | | |
| 144 | + | + | | |
| 145 | ++ | ++ | | |
| 147 | ++ | + | | |
| 148 | ++ | ++ | | |
| 149 | ++ | ++ | | |
| 152 | + | + | | |
| 153 | + | + | | |
| 154 | + | + | | |
| 156 | + | + | | |
| 157 | ++ | + | | |
| 158 | ++ | ++ | | |
| 159 | ++ | + | | |
| 160 | ++ | + | | |
| 161 | + | + | | |
| 166 | + | + | | |
| 168 | +++ | ++ | | |
| 169 | ++ | ++ | | |
| 173 | + | + | | |
| 174 | + | | | |
| 177 | ++++ | | | |
| 179 | + | | | |
| 180 | ++ | | | |
| 181 | + | | | |

For Table 2, “+” refers to an IC₅₀ less than or equal to 100 nM; “++” refers to an IC₅₀ greater than 100 nM and less than or equal to 300 nM; “++++” refers to an IC₅₀ greater than 300 nM and less than or equal to 600 nM; and “++++” refers to an IC₅₀ greater than 600 nM.

### Biochemical Assay for LRRK2 (SEQ. ID NO. 3)

Activity of LRRK2 kinase was determined spectroscopically using a coupled pyruvate kinase/lactate dehydrogenase assay that continuously monitors the ATP hydrolysis-dependent oxidation of NADH (e.g., Schindler et al. Science (2000) 289: 1938-1942). Assays were conducted in 384-well plates (100 μL final volume) using 26.4 nM LRRK2 (Thermo Fisher), 0.1 mM peptide substrate (RLGRDKYKTLRQIRQ), 1.5 units pyruvate kinase, 2.1 units lactate dehydrogenase, 1 mM phosphoenol pyruvate, 0.28 mM NADH and 1 mM ATP in assay buffer (100 mM Tris, pH 7.5, 15 mM MgCl₂, 0.5 mM DTT, 0.004% (w/v) BSA, and 0.004% Triton X-100). Inhibition of LRRK2 was measured by adding serial diluted test compound (final assay concentration of 1% DMSO). A decrease in absorption at 340 nm was monitored continuously for 6 h at 30° C. on a multi-mode microplate reader (BioTek). The reaction rate was calculated using the 2-3 h time frame. The reaction rate at each concentration of compound was converted to percent inhibition using controls (i.e., reaction with no test compound and reaction with a known inhibitor) and IC₅₀ values were calculated using software routines in Prism (GraphPad software).

LRRK2 protein sequence
(residues 970-2528; SEQ. ID NO. 3)
MAPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPKSDLVPRHNQTSLYKKAGTMHSDSISSL
ASEREYITSLDLSANELRDIDALSQKCCISVHLEHLEKLELHQNALTSF
PQQLCETLKSLTHLDLHSNKFTSFPSYLLKMSCIANLDVSRNDIGPSVV
LDPTVKCPTLKQFNLSYNQLSFVPENLTDVVEKLEQLILEGNKISGICS
PLRLKELKILNLSKNHISSLSENFLEACPKVESFSARMNFLAAMPFLPP
SMTILKLSQNKFSCIPEAILNLPHLRSLDMSSNDIQYLPGPAHWKSLNL
RELLFSHNQISILDLSEKAYLWSRVEKLHLSHNKLKEIPPEIGCLENLT
SLDVSYNLELRSFPNEMGKLSKIWDLPLDELHLNFDFKHIGCKAKDIIR
FLQQRLKKAVPYNRMKLMIVGNTGSGKTTLLQQLMKTKKSDLGMQSATV
GIDVKDWPIQIRDKRKRDLVLNVWDFAGREEFYSTHPHFMTQRALYLAV
YDLSKGQAEVDAMKPWLFNIKARASSSPVILVGTHLDVSDEKORKACMS
KITKELLNKRGFPAIRDYHFVNATEESDALAKLRKTIINESLNFKIRDQ
LVVGQLIPDCYVELEKIILSERKNVPIEFPVIDRKRLLQLVRENQLQLD
ENELPHAVHFLNESGVLLHFQDPALQLSDLYFVEPKWLCKIMAQILTVK
VEGCPKHPKGIISRRDVEKFLSKKRKFPKNYMSQYFKLLEKFQIALPIG
EEYLLVPSSLSDHRPVIELPHCENSEIIIRLYEMPYFPMGFWSRLINRL
LEISPYMLSGRERALRPNRMYWRQGIYLNWSPEAYCLVGSEVLDNHPES
FLKITVPSCRKGCILLGQVVDHIDSLMEEWFPGLLEIDICGEGETLLKK
WALYSFNDGEEHQKILLDDLMKKAEEGDLLVNPDQPRLTIPISQIAPDL
ILADLPRNIMLNNDELEFEQAPEFLLGDGSFGSVYRAAYEGEEVAVKIF
NKHTSLRLLRQELVVLCHLHHPSLISLLAAGIRPRMLVMELASKGSLDR
LLQQDKASLTRTLQHRIALHVADGLRYLHSAMIIYRDLKPHNVLLFTLY
PNAAIIAKIADYGIAQYCCRMGIKTSEGTPGFRAPEVARGNVIYNQQAD
VYSFGLLLYDILTTGGRIVEGLKFPNEFDELEIQGKLPDPVKEYGCAPW
PMVEKLIKQCLKENPQERPTSAQVFDILNSAELVCLTRRILLPKNVIVE
CMVATHHNSRNASIWLGCGHTDRGQLSFLDLNTEGYTSEEVADSRILCL
ALVHLPVEKESWIVSGTQSGTLLVINTEDGKKRHTLEKMTDSVTCLYCN
SFSKQSKQKNFLLVGTADGKLAIFEDKTVKLKGAAPLKILNIGNVSTPL
MCLSESTNSTERNVMWGGCGTKIFSFSNDFTIQKLIETRTSQLFSYAAF
SDSNIITVVVDTALYIAKQNSPVVEVWDKKTEKLCGLIDCVHFLREVMV
KENKESKHKMSYSGRVKTLCLQKNTALWIGTGGGHILLLDLSTRRLIRV
IYNFCNSVRVMMTAQLGSLKNVMLVLGYNRKNTEGTQKQKEIQSCLTVW
DINLPHEVQNLEKHIEVRKELAEKMRRTSVE

TABLE 3
Inhibition of LRRK2 kinase activity by exemplary compounds.

| Example | |
|---|---|
| (Compound) | |
| Number | LRRK2 |
| 1 | ++ |
| 4 | + |
| 8 | ++ |
| 26 | ++ |
| 31 | + |
| 37 | ++ |
| 47 | ++ |
| 52 | ++ |
| 54 | +++ |
| 55 | ++ |
| 58 | +++ |
| 62 | + |
| 75 | + |
| 77 | + |
| 78 | + |
| 84 | + |
| 85 | + |
| 86 | ++ |
| 92 | +++ |
| 93 | + |
| 94 | ++ |
| 95 | + |
| 98 | ++ |
| 104 | + |
| 107 | + |
| 108 | ++ |
| 112 | + |
| 113 | ++ |
| 114 | + |
| 116 | + |
| 117 | + |
| 118 | + |
| 119 | + |
| 123 | ++ |
| 125 | + |
| 130 | + |
| 135 | ++ |
| 136 | +++ |
| 137 | ++ |
| 140 | + |
| 142 | + |
| 152 | + |
| 152 | + |
| 154 | + |
| 161 | ++ |
| 173 | + |
| 177 | + |
| 179 | + |
| 180 | + |
| 181 | + |

For Table 3, “+” refers to an IC₅₀ less than or equal to 100 nM; “++” refers to an IC₅₀ greater than 100 nM and less than or equal to 300 nM; “++++” refers to an IC₅₀ greater than 300 nM and less than or equal to 600 nM; and “++++” refers to an IC₅₀ greater than 600 nM.

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained.

What is claimed is:

1. A compound represented by Formula I: 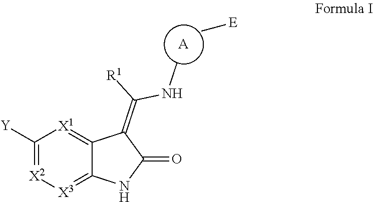
or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
provided that not more than two of X¹, X², and X³ are N;
A is selected from the group consisting of
optionally substituted heteroaryl wherein the optionally substituted substituent, at each occurrence, is independently selected from the group consisting of alkyl, alkoxy, halogen, haloalkyl, haloalkoxy, hydroxy, and cyano, and
optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl;
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
Y is selected from the group consisting of 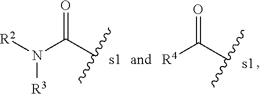
wherein s1 is the site covalently linked to the ring;
R¹ is alkyl or haloalkyl;
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H,
or when Y is 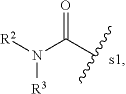
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano; and
R⁴ is selected from the group consisting of
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano;
provided that
when X¹, X², and X³ are CH, Y is 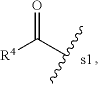
R⁴ is C₁₋₅ alkyl optionally substituted by one or more fluorine atoms, and A is an optionally substituted phenyl, E is not alkyl substituted with amine.

2. A compound represented by Formula II: 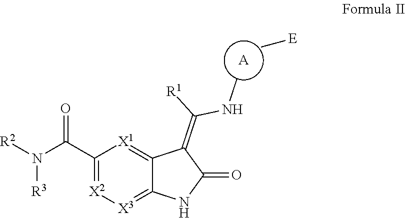
or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
provided that not more than two of X¹, X², and X³ are N;
A is selected from the group consisting of
optionally substituted heteroaryl wherein the optionally substituted substituent, at each occurrence, is independently selected from the group consisting of alkyl, alkoxy, halogen, haloalkyl, haloalkoxy, hydroxy, and cyano, and
optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

3. A compound represented by Formula III: 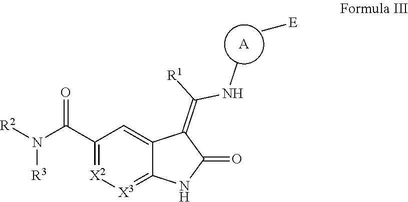
or a pharmaceutically acceptable salt, enantiomer, stereoisomer, or tautomer thereof, wherein:
X³ is selected from the group consisting of N, CH, and CF;
X² is selected from the group consisting of N, CH, CF, and C-Cl;
A is selected from the group consisting of
optionally substituted heteroaryl wherein the optionally substituted substituent, at each occurrence, is independently selected from the group consisting of alkyl, alkoxy, halogen, haloalkyl, haloalkoxy, hydroxy, and cyano, and
optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

4. A compound represented by Formula IV: 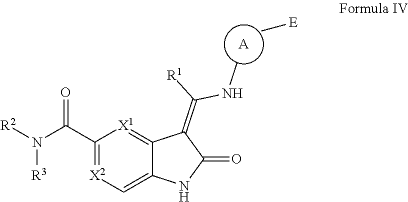
or a pharmaceutically acceptable salt, enantiomer, stereoisomer, or tautomer thereof, wherein:
X¹ is selected from the group consisting of N, CH, and CF;
X² is selected from the group consisting of N, CH, CF, and C-Cl;
A is selected from the group consisting of
optionally substituted heteroaryl wherein the optionally substituted substituent, at each occurrence, is independently selected from the group consisting of alkyl, alkoxy, halogen, haloalkyl, haloalkoxy, hydroxy, and cyano, and
optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

5. A compound represented by Formula V: 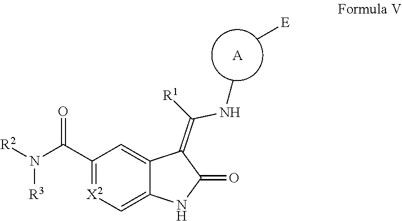
or a pharmaceutically acceptable salt, enantiomer, stereoisomer, or tautomer thereof, wherein:
X² is selected from the group consisting of N, CH, CF, and C-Cl;
A is selected from the group consisting of
optionally substituted heteroaryl wherein the optionally substituted substituent, at each occurrence, is independently selected from the group consisting of alkyl, alkoxy, halogen, haloalkyl, haloalkoxy, hydroxy, and cyano, and
optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

6. A compound represented by Formula VI: 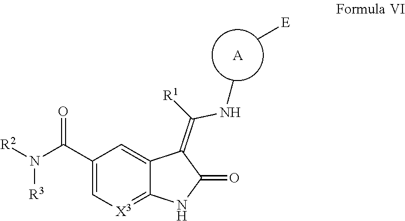
or a pharmaceutically acceptable salt, enantiomer, stereoisomer, or tautomer thereof, wherein:
X³ is selected from the group consisting of N, CH, and CF;
A is selected from the group consisting of
optionally substituted heteroaryl wherein the optionally substituted substituent, at each occurrence, is independently selected from the group consisting of alkyl, alkoxy, halogen, haloalkyl, haloalkoxy, hydroxy, and cyano, and
optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

7. A compound represented by Formula VII: 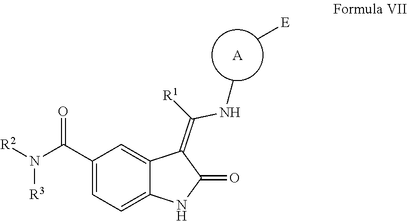
or a pharmaceutically acceptable salt, enantiomer, stereoisomer, or tautomer thereof, wherein:
A is selected from the group consisting of
optionally substituted heteroaryl wherein the optionally substituted substituent, at each occurrence, is independently selected from the group consisting of alkyl, alkoxy, halogen, haloalkyl, haloalkoxy, hydroxy, and cyano, and
optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

8. A compound represented by Formula VIII: 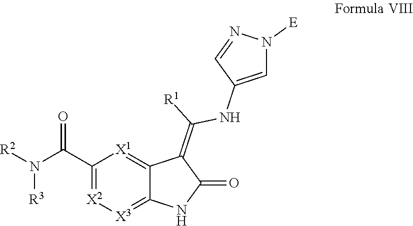
or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
provided that not more than two of X¹, X², and X³ are N;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

9. A compound represented by Formula IX: 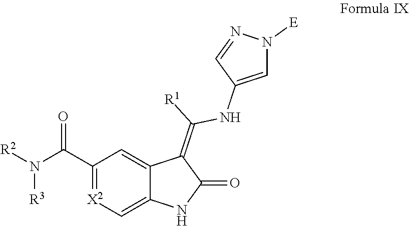
or a pharmaceutically acceptable salt, enantiomer, stereoisomer, or tautomer thereof, wherein:
X² is selected from the group consisting of N, CH, CF, and C-Cl;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

10. A compound represented by Formula X: 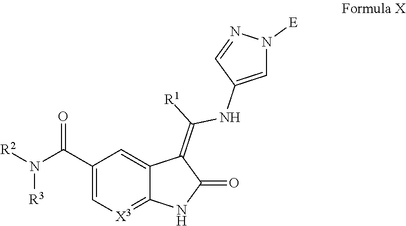
or a pharmaceutically acceptable salt, enantiomer, stereoisomer, or tautomer thereof, wherein:
X³ is selected from the group consisting of N, CH, and CF;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

11. A compound represented by Formula XI: 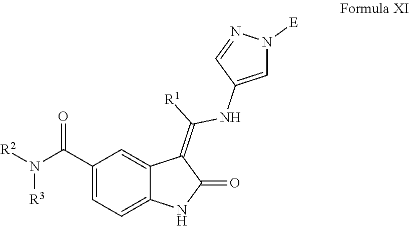
or a pharmaceutically acceptable salt, enantiomer, stereoisomer, or tautomer thereof, wherein:
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

12. A compound represented by Formula XII: 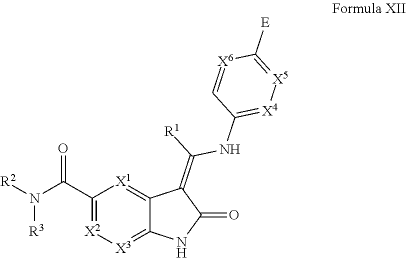
or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
provided that not more than two of X¹, X², and X³ are N;
X⁴, X⁵, and X⁶ are each independently selected from the group consisting of N, CH, and CF;
provided that not more than two of X⁴, X⁵, and X⁶ are N:
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

13. A compound represented by Formula XIII: 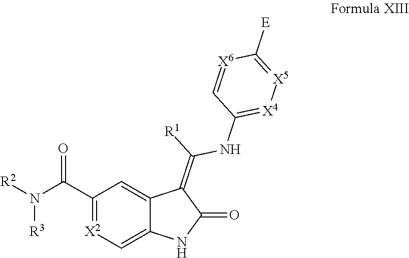
or a pharmaceutically acceptable salt, enantiomer, stereoisomer, or tautomer thereof, wherein:
X² is selected from the group consisting of N, CH, CF, and C-Cl;
X⁴, X⁵, and X⁶ are each independently selected from the group consisting of N, CH, and CF;
provided that not more than two of X⁴, X⁵, and X⁶ are N;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

14. A compound represented by Formula XIV: 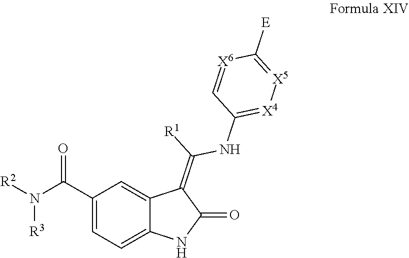
or a pharmaceutically acceptable salt, enantiomer, stereoisomer, or tautomer thereof, wherein:
X⁴, X⁵, and X⁶ are each independently selected from the group consisting of N, CH, and CF;
provided that not more than two of X⁴, X⁵, and X⁶ are N;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R² and R³ are each independently selected from the group consisting of H, alkoxy, haloalkyl,
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano,
provided that when one of R² and R³ is H, the other of R² and R³ is not H, or
R² and R³ when together with the nitrogen atom to which they are attached form an optionally substituted heterocyclyl, wherein the optionally substituted substituent, at each occurrence, is independently selected from the group consisting of alkyl, amine, alkoxy, halogen, haloalkyl, haloalkoxy, hydroxy, and cyano.

15. A compound represented by Formula XV: 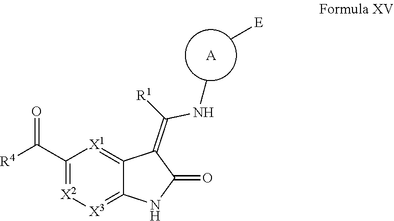
or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
provided that not more than two of X¹, X², and X³ are N;
A is selected from the group consisting of
optionally substituted heteroaryl wherein the optionally substituted substituent, at each occurrence, is independently selected from the group consisting of alkyl, alkoxy, halogen, haloalkyl, haloalkoxy, hydroxy, and cyano, and
optionally substituted phenyl wherein the optionally substituted substituent, at each occurrence, is independently selected from the group consisting of alkyl, alkoxy, halogen, haloalkyl, haloalkoxy, hydroxy, and cyano;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R⁴ is selected from the group consisting of
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano;
provided that
when X¹, X², and X³ are CH, R⁴ is C₁₋₅ alkyl optionally substituted by one or more fluorine atoms, and A is an optionally substituted phenyl, E is not alkyl substituted with amine.

16. A compound represented by Formula XVI: 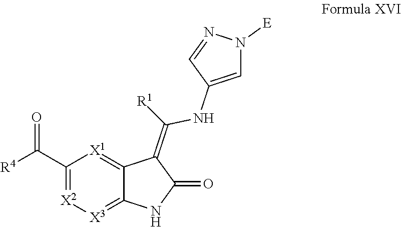
or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
provided that not more than two of X¹, X², and X³ are N;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R⁴ is selected from the group consisting of
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano.

17. A compound represented by Formula XVII: 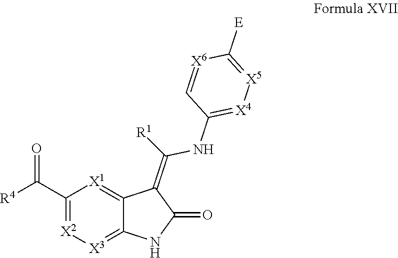
or a pharmaceutically acceptable salt, enantiomer, stereoisomer, or tautomer thereof, wherein:
X¹ and X³ are each independently selected from the group consisting of N, CH, and CF;
X² is selected from the group consisting of N, CH, CF, and C-Cl;
provided that not more than two of X¹, X², and X³ are N;
X⁴, X⁵, and X⁶ are each independently selected from the group consisting of N, CH, and CF;
provided that not more than two of X⁴, X⁵, and X⁶ are N;
E is selected from the group consisting of H, haloalkyl,
optionally substituted alkyl wherein the optionally substituted substituent, at each occurrence, is independently selected from the group consisting of amine, alkoxy, halogen, haloalkyl, haloalkoxy, hydroxy, and cyano,
optionally substituted cycloalkyl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl,
optionally substituted heteroaryl wherein the optionally substituted substituent, at each occurrence, is independently selected from the group consisting of alkyl, amine, alkoxy, halogen, haloalkyl, haloalkoxy, hydroxy, and cyano, wherein the optional alkyl is further optionally substituted with alkoxy or cycloalkyl, and
optionally substituted heterocyclyl wherein the optionally substituted substituent, at each occurrence, is independently selected from the group consisting of alkyl, cycloalkyl, heterocyclyl, amine, alkoxy, halogen, haloalkyl, haloalkoxy, hydroxy, and cyano, wherein the optional alkyl is further optionally substituted with alkoxy or cycloalkyl;
R¹ is alkyl or haloalkyl; and
R⁴ is selected from the group consisting of
optionally substituted alkyl wherein the optionally substituted substituent, at each occurrence, is independently selected from the group consisting of alkoxy, amine, amide, halogen, haloalkyl, haloalkoxy, hydroxy, cyano, cycloalkyl, heterocyclyl, and heteroaryl, wherein the cycloalkyl, heterocyclyl, or heteroaryl is optionally substituted with alkyl, alkoxy, amine, amide, halogen, haloalkyl, haloalkoxy, hydroxy, cyano, cycloalkyl, heterocyclyl, or heterocyclyl,
optionally substituted cycloalkyl wherein the optionally substituted substituent, at each occurrence, is independently selected from the group consisting of alkyl, alkoxy, amine, amide, halogen, haloalkyl, haloalkoxy, hydroxy, and cyano,
optionally substituted c-linked heterocyclyl wherein the optionally substituted substituent, at each occurrence, is independently selected from the group consisting of alkyl, alkoxy, amine, amide, halogen, haloalkyl, haloalkoxy, hydroxy, and cyano,
optionally substituted heteroaryl wherein the optionally substituted substituent, at each occurrence, is independently selected from the group consisting of alkyl, alkoxy, amine, amide, halogen, haloalkyl, haloalkoxy, hydroxy, and cyano;
provided that when X¹, X², X³, X⁴, X⁵, and X⁶ are CH, and R⁴ is C₁₋₅ alkyl optionally substituted by one or more fluorine atoms, E is not alkyl substituted with amine.

18. The compound of any one of claims 1, 2, 4, 8, 12, and 15-17, wherein X¹ is CH.

19. The compound of any one of claims 1, 2, 4, 8, 12, and 15-17, wherein X¹ is CF or N.

20. The compound of any one of claims 1-3, 6, 8, 10, 12, and 15-17, wherein X³ is CH.

21. The compound of any one of claims 1-3, 6, 8, 10, 12, and 15-17, wherein X³ is CF or N.

22. The compound of any one of claims 1-5, 8, 9, 12, 13, and 15-17, wherein X² is CH.

23. The compound of any one of claims 1-5, 8, 9, 12, 13, and 15-17, wherein X² is CF, C-Cl, or N.

24. The compound of any one of claims 1, 2, 8, 12, and 15-17, wherein X¹, X², and X³ are CH.

25. The compound of any one of claims 1-24, wherein R¹ is alkyl.

26. The compound of any one of claims 1-25, wherein R¹ is methyl.

27. The compound of any one of claims 1-25, wherein R¹ is ethyl.

28. The compound of any one of claims 1-7 and 15, wherein A is selected from the group consisting of 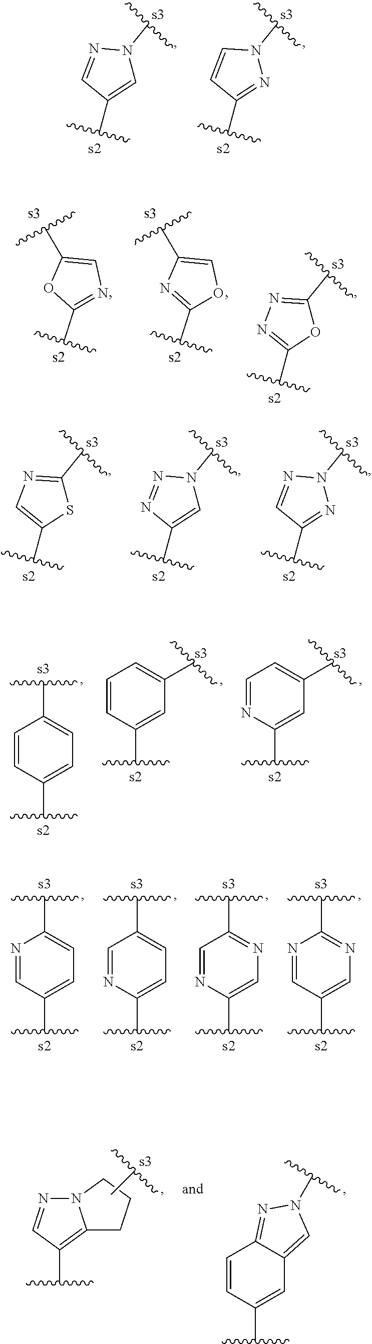
each of which is optionally substituted with alkyl, alkoxy, halogen, haloalkyl, haloalkoxy, hydroxy, or cyano, wherein s2 is the site covalently linked to —NH— and s3 is the site covalently linked to E.

29. The compound of any one of claims 1-7 and 15, wherein A is phenyl optionally substituted with halogen or alkyl.

30. The compound of any one of claims 1-7 and 15, wherein A is 5-6 membered heteroaryl optionally substituted with halogen or alkyl.

31. The compound of any one of claims 1-7, 15, and 30, wherein A is pyrazole optionally substituted with halogen or alkyl.

32. The compound of any one of claims 1-14 and 18-31, wherein R² is H.

33. The compound of any one of claims 1-14 and 18-32, wherein R² is H and R³ is alkoxy.

34. The compound of any one of claims 1-14 and 18-32, wherein R² is H and R³ is haloalkyl.

35. The compound of any one of claims 1-14 and 18-32, wherein R² is H and R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl.

36. The compound of any one of claims 1-14 and 18-32, wherein R² is H and R³ is cycloalkyl optionally substituted with one or more alkyl, alkoxy, amine, amide, halogen, haloalkyl, haloalkoxy, hydroxy, or cyano.

37. The compound of any one of claims 1-14, 18-32, and 36, wherein R² is H and R³ is cycloalkyl optionally substituted with one or more halogen.

38. The compound of any one of claims 1-14 and 18-32, wherein R² is H and R³ is heteroaryl optionally substituted with one or more alkyl, alkoxy, amine, amide, halogen, haloalkyl, haloalkoxy, hydroxy, or cyano.

39. The compound of any one of claims 1-14, 18-32, and 38, wherein R² is H and R³ is heteroaryl optionally substituted with haloalkyl.

40. The compound of any one of claims 1-14 and 18-32, wherein R² is H and R³ is heterocyclyl optionally substituted with one or more alkyl, alkoxy, amine, amide, halogen, haloalkyl, haloalkoxy, hydroxy, or cyano.

41. The compound of any one of claims 1-14, 18-32, and 40, wherein R² is H and R³ is heterocyclyl.

42. The compound of any one of claims 1-14 and 18-32, wherein R² is H and R³ is selected from the group consisting of methyl, ethyl, n-propyl, isopropyl,

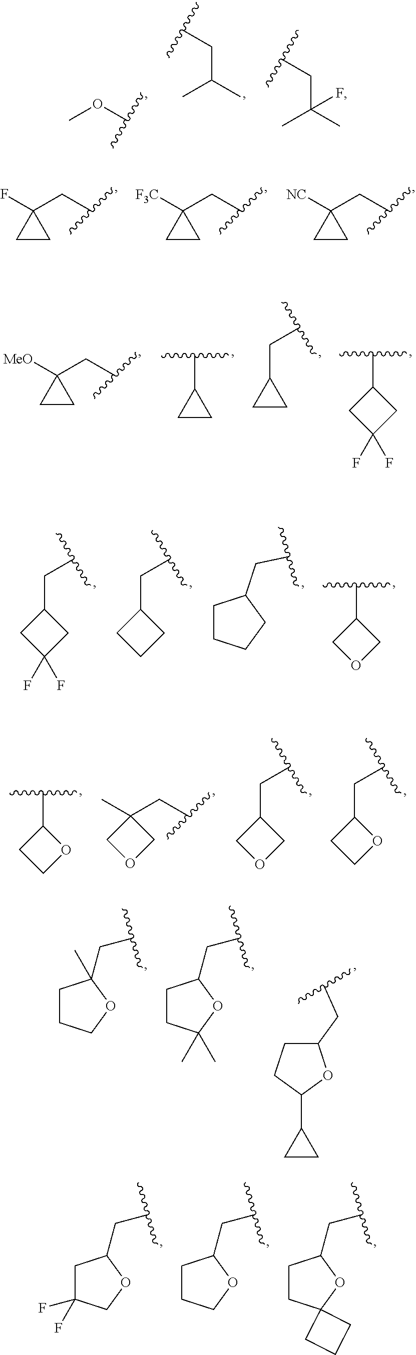

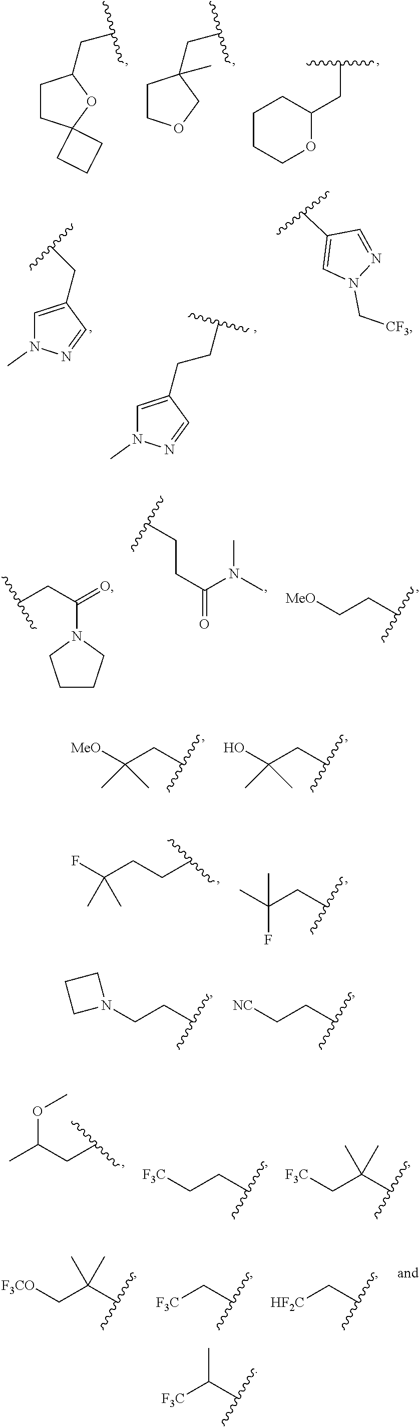

43. The compound of any one of claims 1-14 and 18-31, wherein R² is alkyl.

44. The compound of any one of claims 1-14, 18-31, and 43, wherein R² is methyl.

45. The compound of any one of claims 1-14 and 17-30, wherein R² is cycloalkyl.

46. The compound of any one of claims 1-14, 18-31, and 45, wherein R² is cyclopropyl.

47. The compound of any one of claims 1-14, 18-31, and 43-46, wherein R³ is alkoxy.

48. The compound of any one of claims 1-14, 18-31, and 43-46, wherein R³ is haloalkyl.

49. The compound of any one of claims 1-14, 18-31, and 43-46, wherein R³ is alkyl optionally substituted with one or more alkoxy, amine, amide, halogen, haloalkyl, haloalkoxy, hydroxy, cyano, cycloalkyl, heterocyclyl, or heteroaryl, wherein the cycloalkyl, heterocyclyl, or heteroaryl is optionally substituted with one or more halogen, alkyl, halkoalkyl, cyano, or cycloalkyl.

50. The compound of any one of claims 1-14, 18-31, and 43-46, wherein R³ is cycloalkyl optionally substituted with one or more alkyl, alkoxy, amine, amide, halogen, haloalkyl, haloalkoxy, hydroxy, or cyano.

51. The compound of any one of claims 1-14, 18-31, 43-46, and 50, wherein R³ is cycloalkyl optionally substituted with one or more halogen.

52. The compound of any one of claims 1-14, 18-31, and 43-46, wherein R³ is heteroaryl optionally substituted with one or more alkyl, alkoxy, amine, amide, halogen, haloalkyl, haloalkoxy, hydroxy, or cyano.

53. The compound of any one of claims 1-14, 18-31, 43-46, and 52, wherein R³ is heteroaryl optionally substituted with haloalkyl.

54. The compound of any one of claims 1-14, 18-31, and 43-46, wherein R³ is heterocyclyl optionally substituted with one or more alkyl, alkoxy, amine, amide, halogen, haloalkyl, haloalkoxy, hydroxy, or cyano.

55. The compound of any one of claims 1-14, 18-31, 43-46, and 54, wherein R³ is heterocyclyl.

56. The compound of any one of claims 1-14, 18-31, and 43-46, wherein R³ is selected from the group consisting of methyl, ethyl, n-propyl, isopropyl,

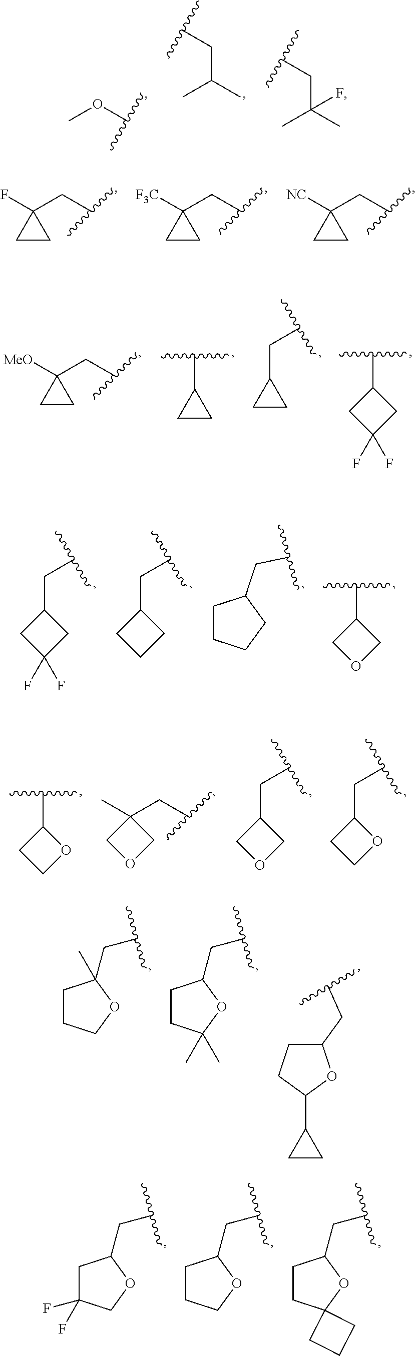

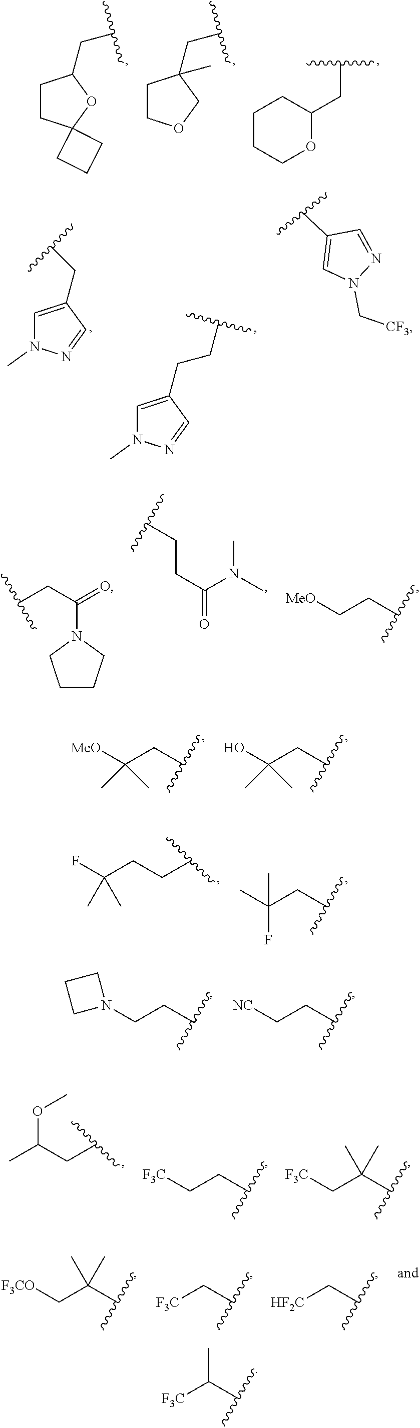

57. The compound of any one of claims 1-14 and 18-31, wherein R² and R³ together with the nitrogen atom to which they are attached form an optionally substituted heterocyclyl selected from the group consisting of

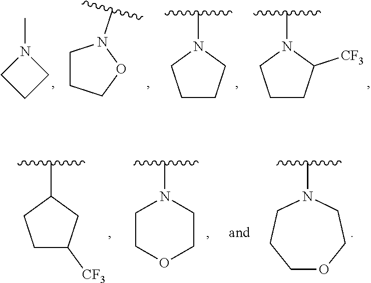

58. The compound of any one of claims 1 and 15-31, wherein R⁴ is selected from the group consisting of optionally substituted alkyl and cycloalkyl.

59. The compound of any one of claims 1, 15-31, and 58, wherein R⁴ is selected from the 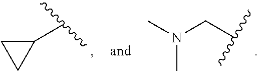
group consisting of methyl,

60. The compound of any one of claims 1-59, wherein E is selected from the group consisting of 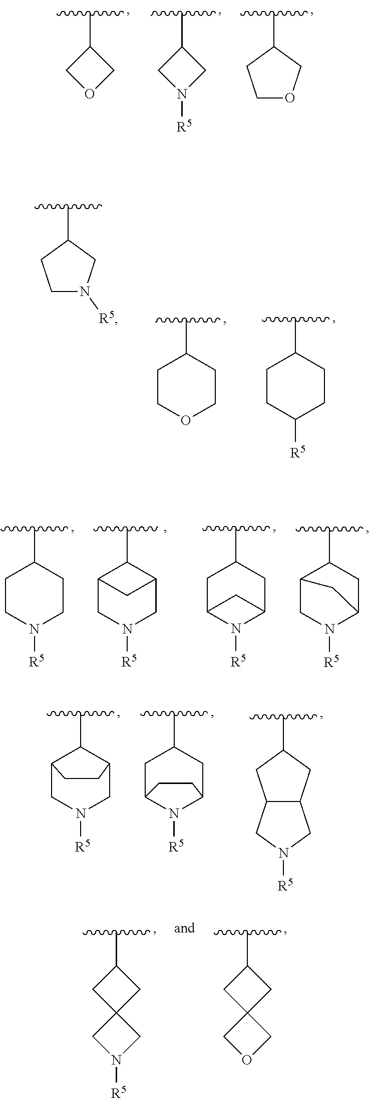
wherein R⁵ is independently H, alkyl, cycloalkyl, haloalkyl, or heterocyclyl.

61. The compound of any one of claims 1-7 and 12-14, wherein E is selected from the group consisting of 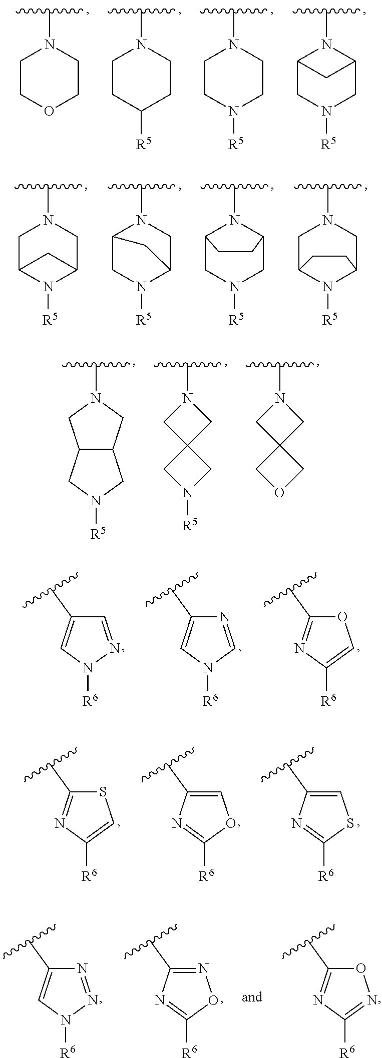
wherein R⁵ is independently H, alkyl, cycloalkyl, haloalkyl, or heterocyclyl, and R⁶ is independently H, alkyl, cycloalkyl, or haloalkyl.

62. The compound of any one of claims 1-59, wherein E is H.

63. The compound of any one of claims 1-59, wherein E is alkyl optionally substituted with cycloalkyl, amine, or alkoxy.

64. The compound of any one of claims 1-59, wherein E is alkyl.

65. The compound of any one of claims 1-59, wherein E is haloalkyl.

66. The compound of any one of claims 1-59, wherein E is cycloalkyl optionally substituted with one or more halogen.

67. The compound of any one of claims 1-59, wherein E is 4-6 membered heterocyclyl.

68. The compound of any one of claims 1-59, wherein E is selected from the group consisting of H, alkyl optionally substituted with cycloalkyl, amine, or alkoxy, haloalkyl, cycloalkyl optionally substituted with one or more halogen, and heterocyclyl.

69. The compound of any one of claims 1-59, wherein E is selected from the group consisting of H, methyl, ethyl, trifluoromethyl, difluoromethyl,

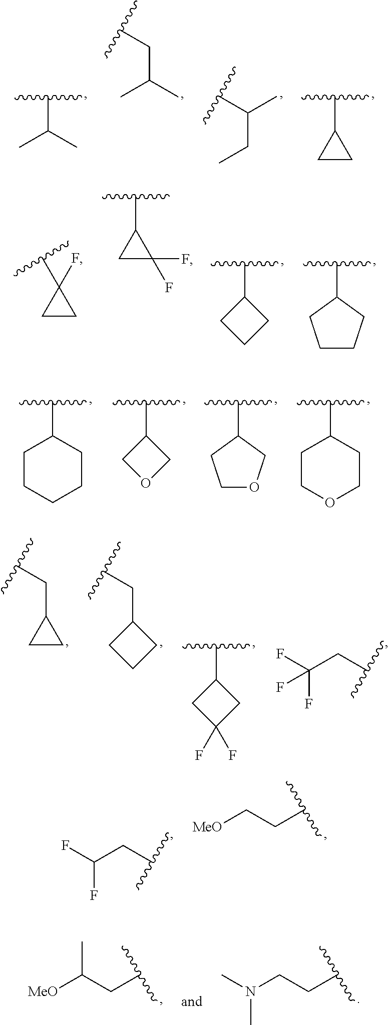

70. A compound selected from the group consisting of: 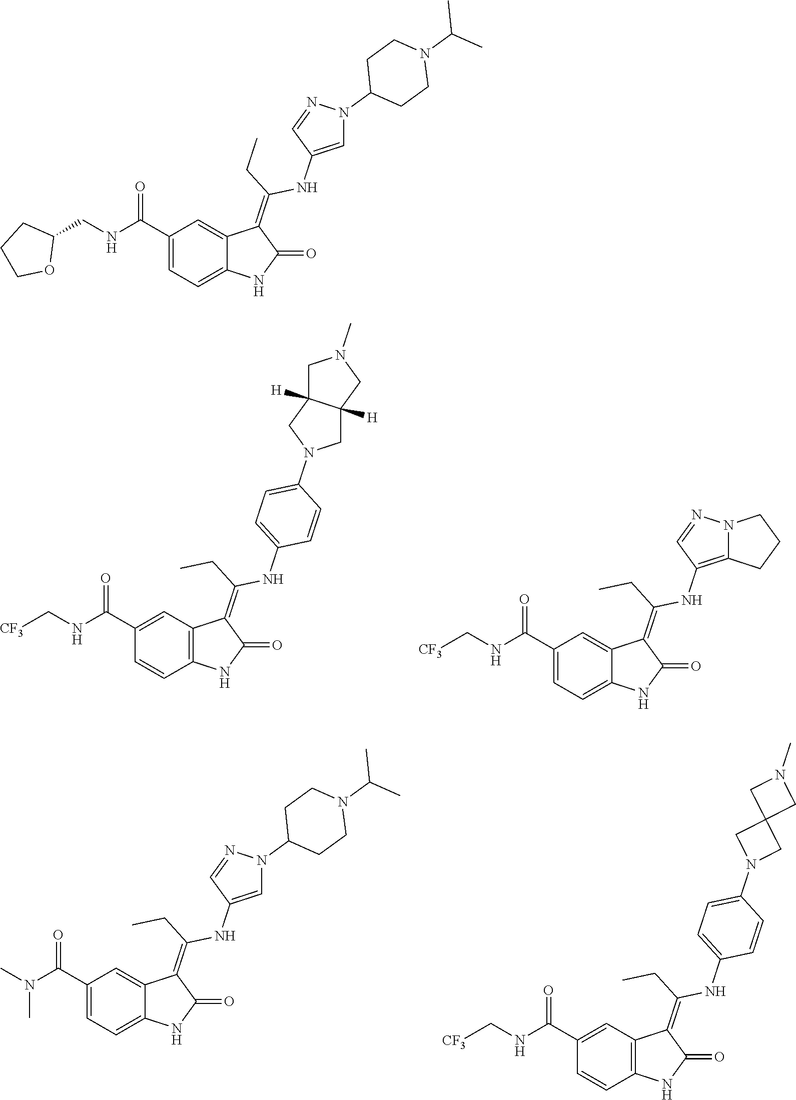 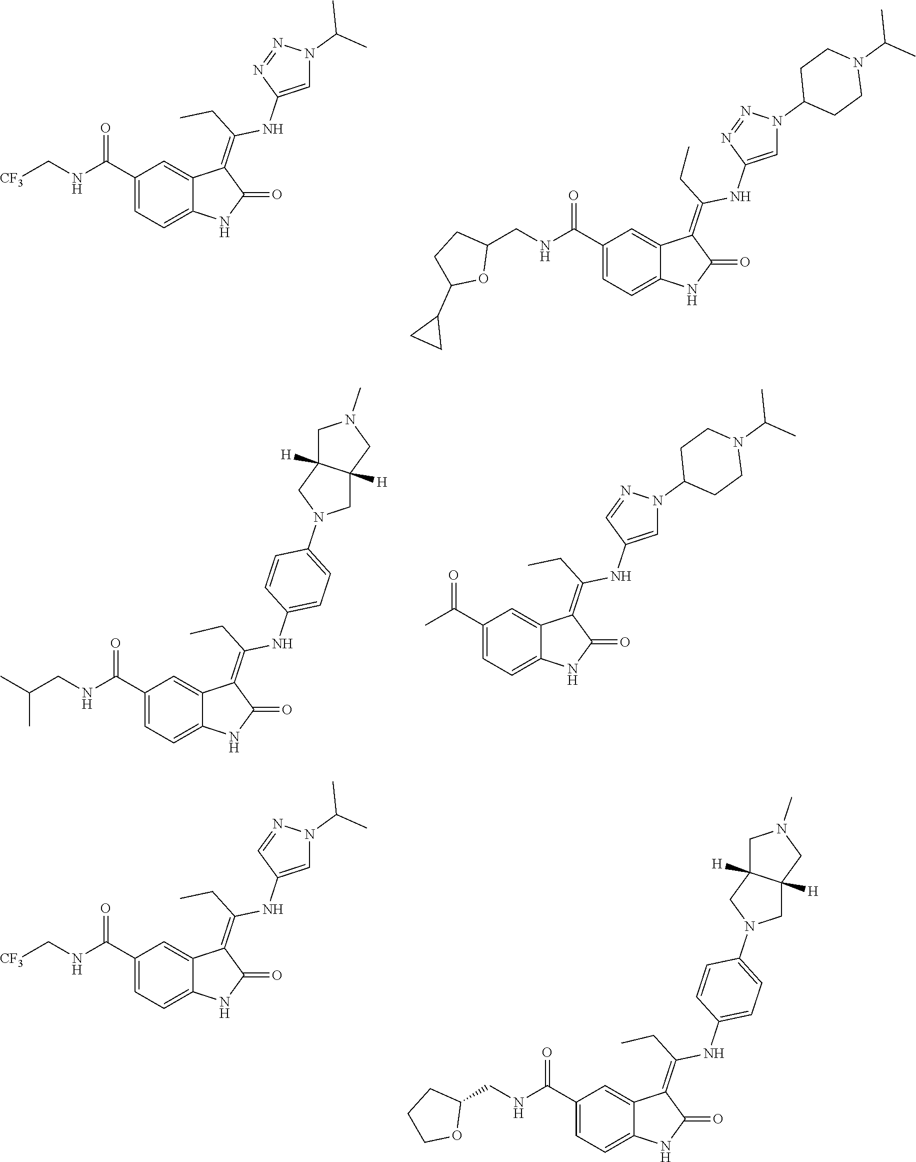 [SMILES: CC/C(Nc1cn(C(C)C)nn1)=C1/C(=O)Nc2ccc(C(=O)NCC(F)(F)F)cc21 ; CC/C(Nc1cn(C2CCN(C(C)C)CC2)nn1)=C1/C(=O)Nc2ccc(C(=O)NCC3CCC(C4CC4)O3)cc21 ; CC/C(Nc1ccc(N2C[C@H]3CN(C)C[C@H]3C2)cc1)=C1/C(=O)Nc2ccc(C(=O)NCC(C)C)cc21 ; CC/C(Nc1cnn(C2CCN(C(C)C)CC2)c1)=C1/C(=O)Nc2ccc(C(C)=O)cc21 ; CC/C(Nc1cnn(C(C)C)c1)=C1/C(=O)Nc2ccc(C(=O)NCC(F)(F)F)cc21 ; CC/C(Nc1ccc(N2C[C@H]3CN(C)C[C@H]3C2)cc1)=C1/C(=O)Nc2ccc(C(=O)NC[C@H]3CCCO3)cc21] 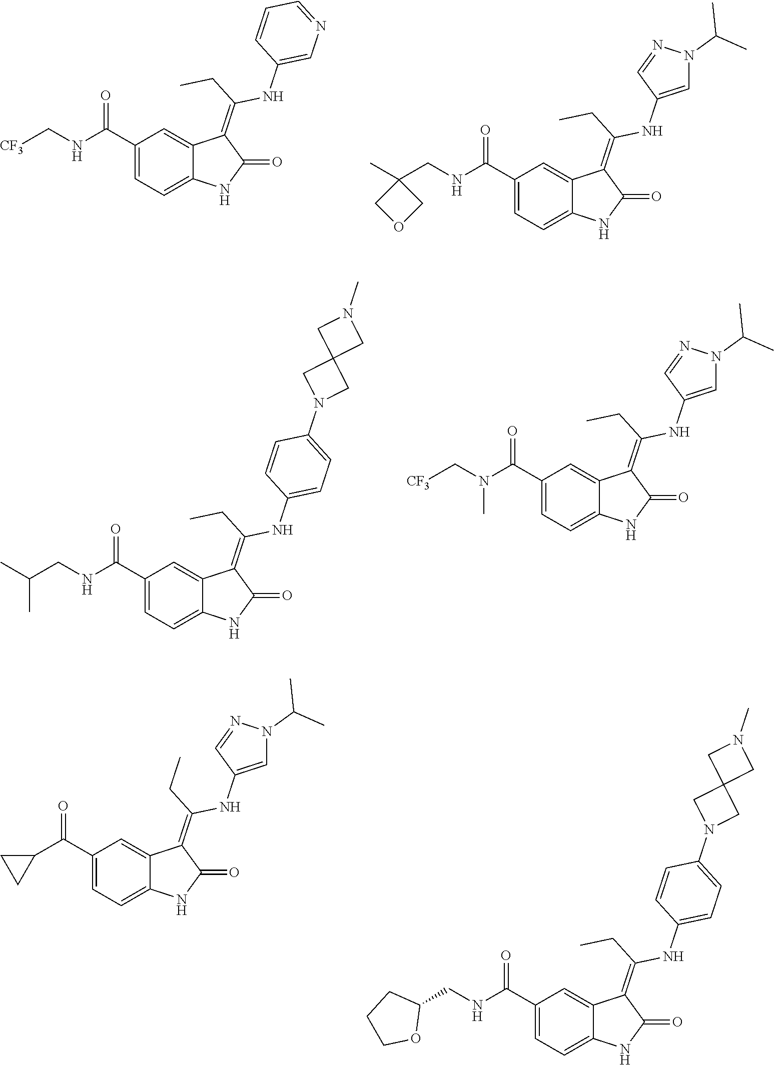 [SMILES: CC/C(Nc1cccnc1)=C1/C(=O)Nc2ccc(C(=O)NCC(F)(F)F)cc21 ; CC/C(Nc1cnn(C(C)C)c1)=C1/C(=O)Nc2ccc(C(=O)NCC3(C)COC3)cc21 ; CC/C(Nc1ccc(N2CC3(CN(C)C3)C2)cc1)=C1/C(=O)Nc2ccc(C(=O)NCC(C)C)cc21 ; CC/C(Nc1cnn(C(C)C)c1)=C1/C(=O)Nc2ccc(C(=O)N(C)CC(F)(F)F)cc21 ; CC/C(Nc1cnn(C(C)C)c1)=C1/C(=O)Nc2ccc(C(=O)C3CC3)cc21 ; CC/C(Nc1ccc(N2CC3(CN(C)C3)C2)cc1)=C1/C(=O)Nc2ccc(C(=O)NC[C@H]3CCCO3)cc21] 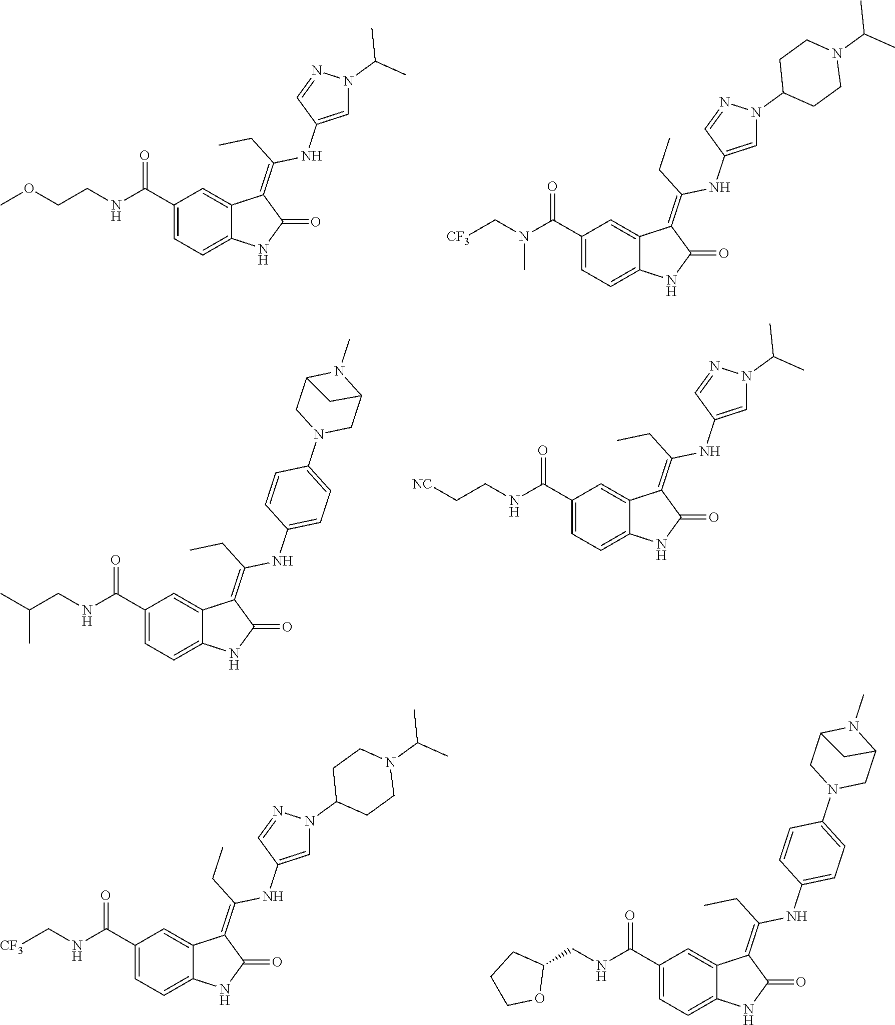 [SMILES: CC/C(Nc1cnn(C(C)C)c1)=C1/C(=O)Nc2ccc(C(=O)NCCOC)cc21 ; CC/C(Nc1cnn(C2CCN(C(C)C)CC2)c1)=C1/C(=O)Nc2ccc(C(=O)N(C)CC(F)(F)F)cc21 ; CC/C(Nc1ccc(N2CC3CC(C2)N3C)cc1)=C1/C(=O)Nc2ccc(C(=O)NCC(C)C)cc21 ; CC/C(Nc1cnn(C(C)C)c1)=C1/C(=O)Nc2ccc(C(=O)NCCC#N)cc21 ; CC/C(Nc1cnn(C2CCN(C(C)C)CC2)c1)=C1/C(=O)Nc2ccc(C(=O)NCC(F)(F)F)cc21 ; CC/C(Nc1ccc(N2CC3CC(C2)N3C)cc1)=C1/C(=O)Nc2ccc(C(=O)NC[C@H]3CCCO3)cc21] 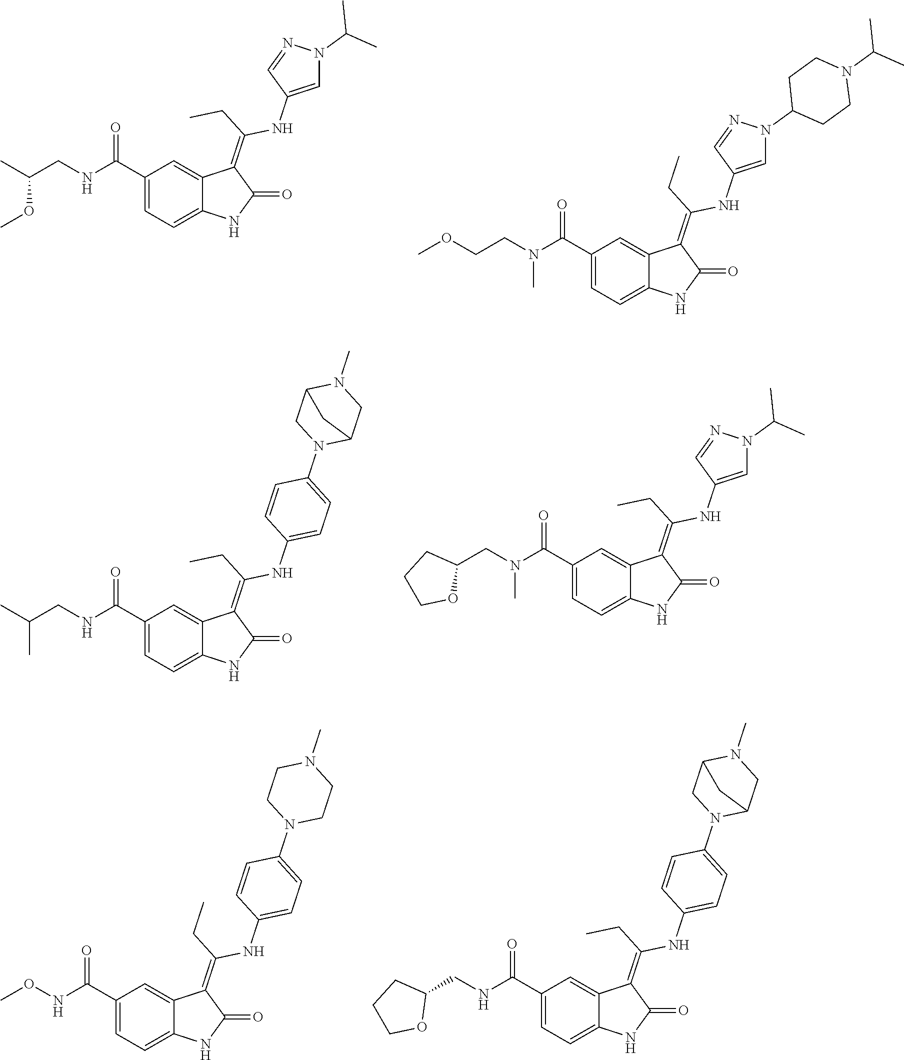 [SMILES: CC/C(Nc1cnn(C(C)C)c1)=C1/C(=O)Nc2ccc(C(=O)NC[C@@H](C)OC)cc21 ; CC/C(Nc1cnn(C2CCN(C(C)C)CC2)c1)=C1/C(=O)Nc2ccc(C(=O)N(C)CCOC)cc21 ; CC/C(Nc1ccc(N2CC3CC2CN3C)cc1)=C1/C(=O)Nc2ccc(C(=O)NCC(C)C)cc21 ; CC/C(Nc1cnn(C(C)C)c1)=C1/C(=O)Nc2ccc(C(=O)N(C)C[C@H]3CCCO3)cc21 ; CC/C(Nc1ccc(N2CCN(C)CC2)cc1)=C1/C(=O)Nc2ccc(C(=O)NOC)cc21 ; CC/C(Nc1ccc(N2CC3CC2CN3C)cc1)=C1/C(=O)Nc2ccc(C(=O)NC[C@H]3CCCO3)cc21] 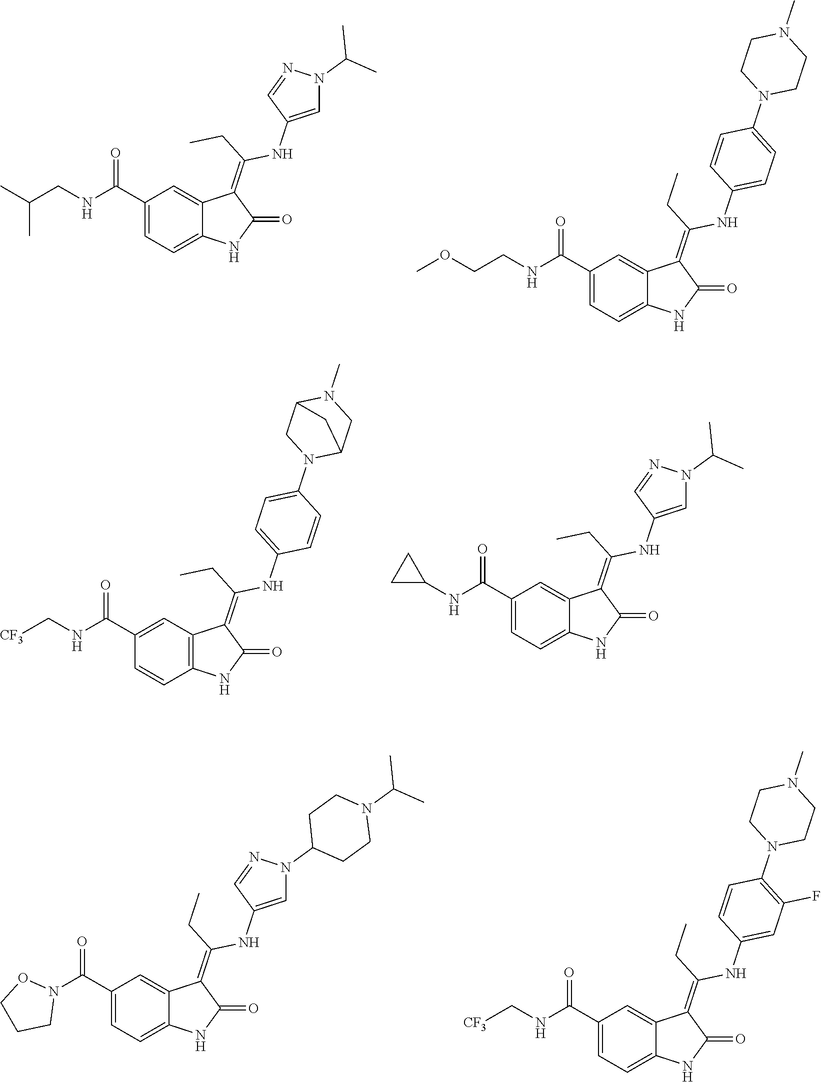 [SMILES: CC/C(Nc1cnn(C(C)C)c1)=C1/C(=O)Nc2ccc(C(=O)NCC(C)C)cc21 ; CC/C(Nc1ccc(N2CCN(C)CC2)cc1)=C1/C(=O)Nc2ccc(C(=O)NCCOC)cc21 ; CC/C(Nc1ccc(N2CC3CC2CN3C)cc1)=C1/C(=O)Nc2ccc(C(=O)NCC(F)(F)F)cc21 ; CC/C(Nc1cnn(C(C)C)c1)=C1/C(=O)Nc2ccc(C(=O)NC3CC3)cc21 ; CC/C(Nc1cnn(C2CCN(C(C)C)CC2)c1)=C1/C(=O)Nc2ccc(C(=O)N3CCCO3)cc21 ; CC/C(Nc1ccc(N2CCN(C)CC2)c(F)c1)=C1/C(=O)Nc2ccc(C(=O)NCC(F)(F)F)cc21] 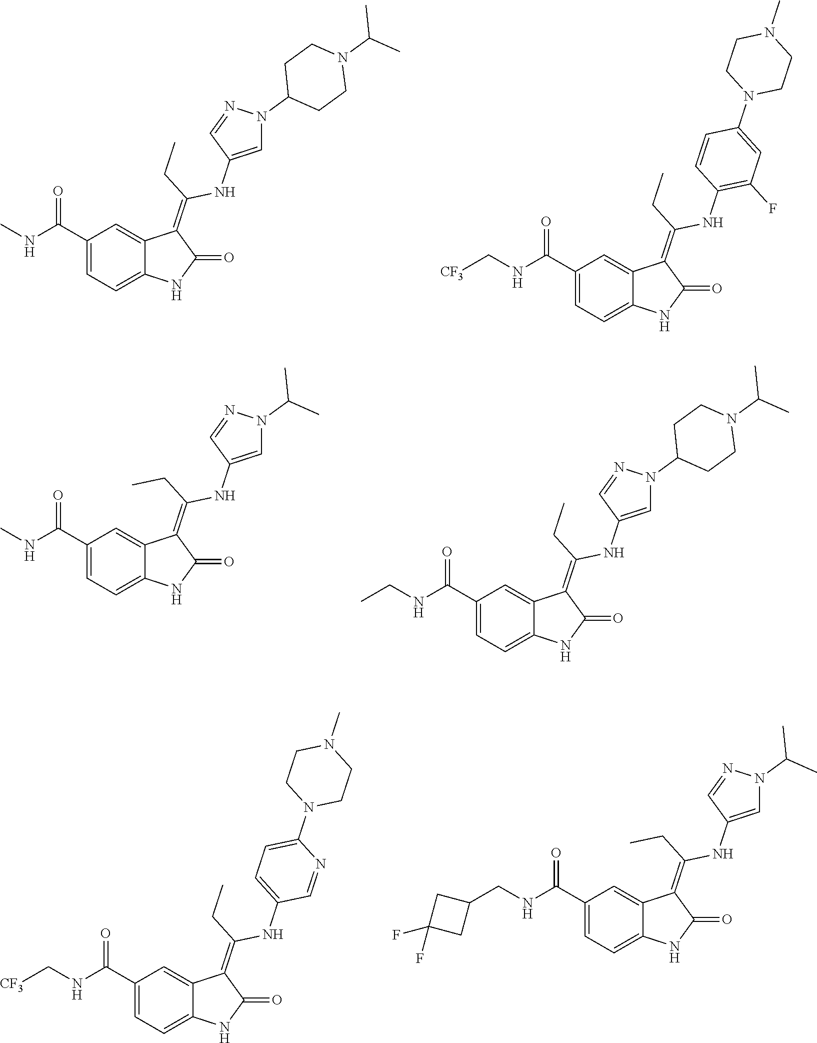 [SMILES: CC/C(Nc1cnn(C2CCN(C(C)C)CC2)c1)=C1/C(=O)Nc2ccc(C(=O)NC)cc21 ; CC/C(Nc1ccc(N2CCN(C)CC2)cc1F)=C1/C(=O)Nc2ccc(C(=O)NCC(F)(F)F)cc21 ; CC/C(Nc1cnn(C(C)C)c1)=C1/C(=O)Nc2ccc(C(=O)NC)cc21 ; CCNC(=O)c1ccc2c(c1)/C(=C(\CC)Nc1cnn(C3CCN(C(C)C)CC3)c1)C(=O)N2 ; CC/C(Nc1ccc(N2CCN(C)CC2)nc1)=C1/C(=O)Nc2ccc(C(=O)NCC(F)(F)F)cc21 ; CC/C(Nc1cnn(C(C)C)c1)=C1/C(=O)Nc2ccc(C(=O)NCC3CC(F)(F)C3)cc21] 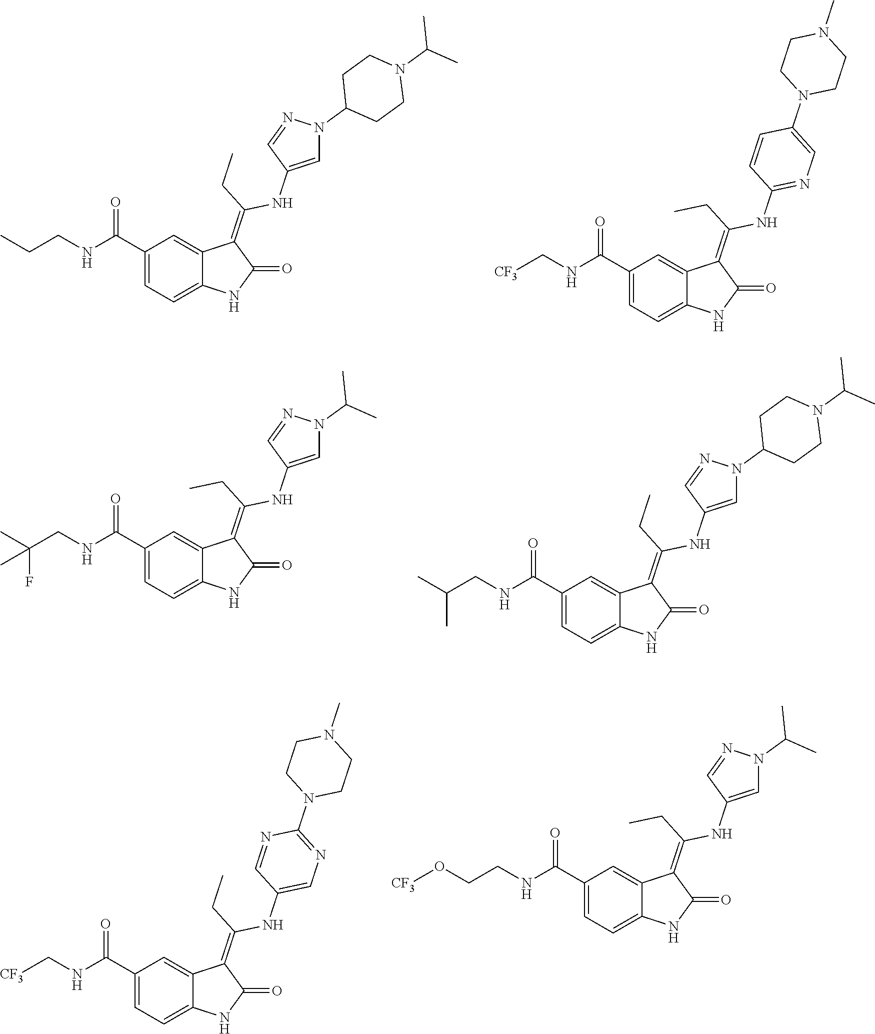 [SMILES: CCCNC(=O)c1ccc2c(c1)/C(=C(\CC)Nc1cnn(C3CCN(C(C)C)CC3)c1)C(=O)N2 ; CC/C(Nc1ccc(N2CCN(C)CC2)cn1)=C1/C(=O)Nc2ccc(C(=O)NCC(F)(F)F)cc21 ; CC/C(Nc1cnn(C(C)C)c1)=C1/C(=O)Nc2ccc(C(=O)NCC(C)(C)F)cc21 ; CC/C(Nc1cnn(C2CCN(C(C)C)CC2)c1)=C1/C(=O)Nc2ccc(C(=O)NCC(C)C)cc21 ; CC/C(Nc1cnc(N2CCN(C)CC2)nc1)=C1/C(=O)Nc2ccc(C(=O)NCC(F)(F)F)cc21 ; CC/C(Nc1cnn(C(C)C)c1)=C1/C(=O)Nc2ccc(C(=O)NCCOC(F)(F)F)cc21] 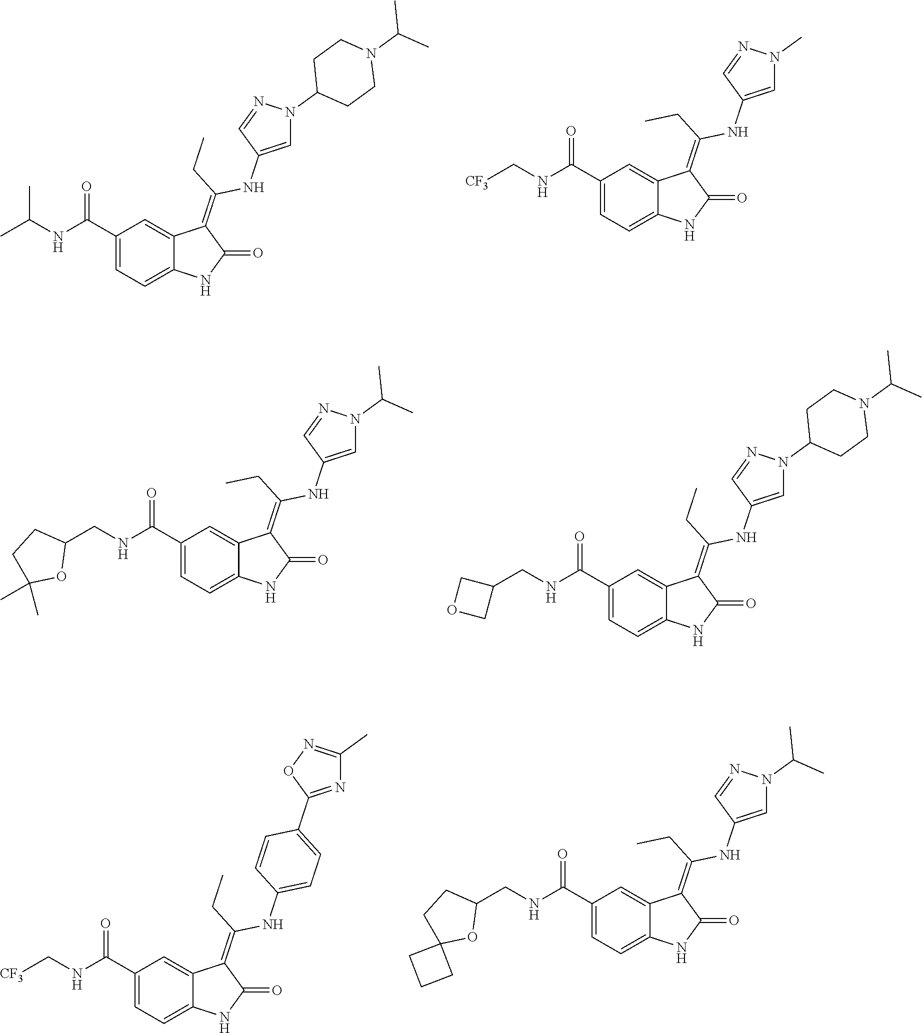 [SMILES: CC/C(Nc1cnn(C2CCN(C(C)C)CC2)c1)=C1/C(=O)Nc2ccc(C(=O)NC(C)C)cc21 ; CC/C(Nc1cnn(C)c1)=C1/C(=O)Nc2ccc(C(=O)NCC(F)(F)F)cc21 ; CC/C(Nc1cnn(C(C)C)c1)=C1/C(=O)Nc2ccc(C(=O)NCC3CCC(C)(C)O3)cc21 ; CC/C(Nc1cnn(C2CCN(C(C)C)CC2)c1)=C1/C(=O)Nc2ccc(C(=O)NCC3COC3)cc21 ; CC/C(Nc1ccc(-c2nc(C)no2)cc1)=C1/C(=O)Nc2ccc(C(=O)NCC(F)(F)F)cc21 ; CC/C(Nc1cnn(C(C)C)c1)=C1/C(=O)Nc2ccc(C(=O)NCC3CCC4(CCC4)O3)cc21] 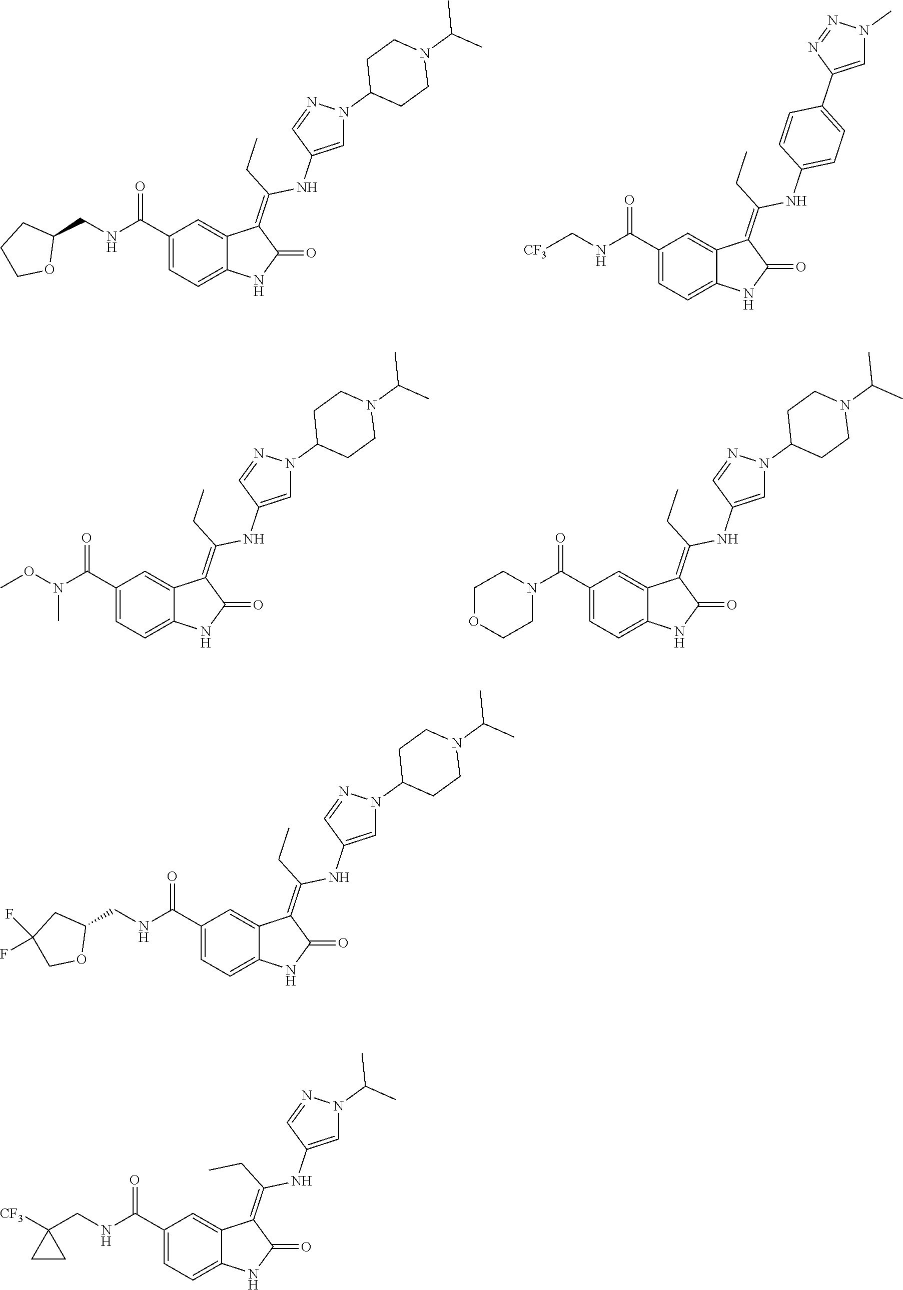 [SMILES: CC/C(Nc1cnn(C2CCN(C(C)C)CC2)c1)=C1/C(=O)Nc2ccc(C(=O)NC[C@@H]3CCCO3)cc21 ; CC/C(Nc1ccc(-c2cn(C)nn2)cc1)=C1/C(=O)Nc2ccc(C(=O)NCC(F)(F)F)cc21 ; CC/C(Nc1cnn(C2CCN(C(C)C)CC2)c1)=C1/C(=O)Nc2ccc(C(=O)N(C)OC)cc21 ; CC/C(Nc1cnn(C2CCN(C(C)C)CC2)c1)=C1/C(=O)Nc2ccc(C(=O)N3CCOCC3)cc21 ; CC/C(Nc1cnn(C2CCN(C(C)C)CC2)c1)=C1/C(=O)Nc2ccc(C(=O)NC[C@H]3CC(F)(F)CO3)cc21 ; CC/C(Nc1cnn(C(C)C)c1)=C1/C(=O)Nc2ccc(C(=O)NCC3(C(F)(F)F)CC3)cc21] 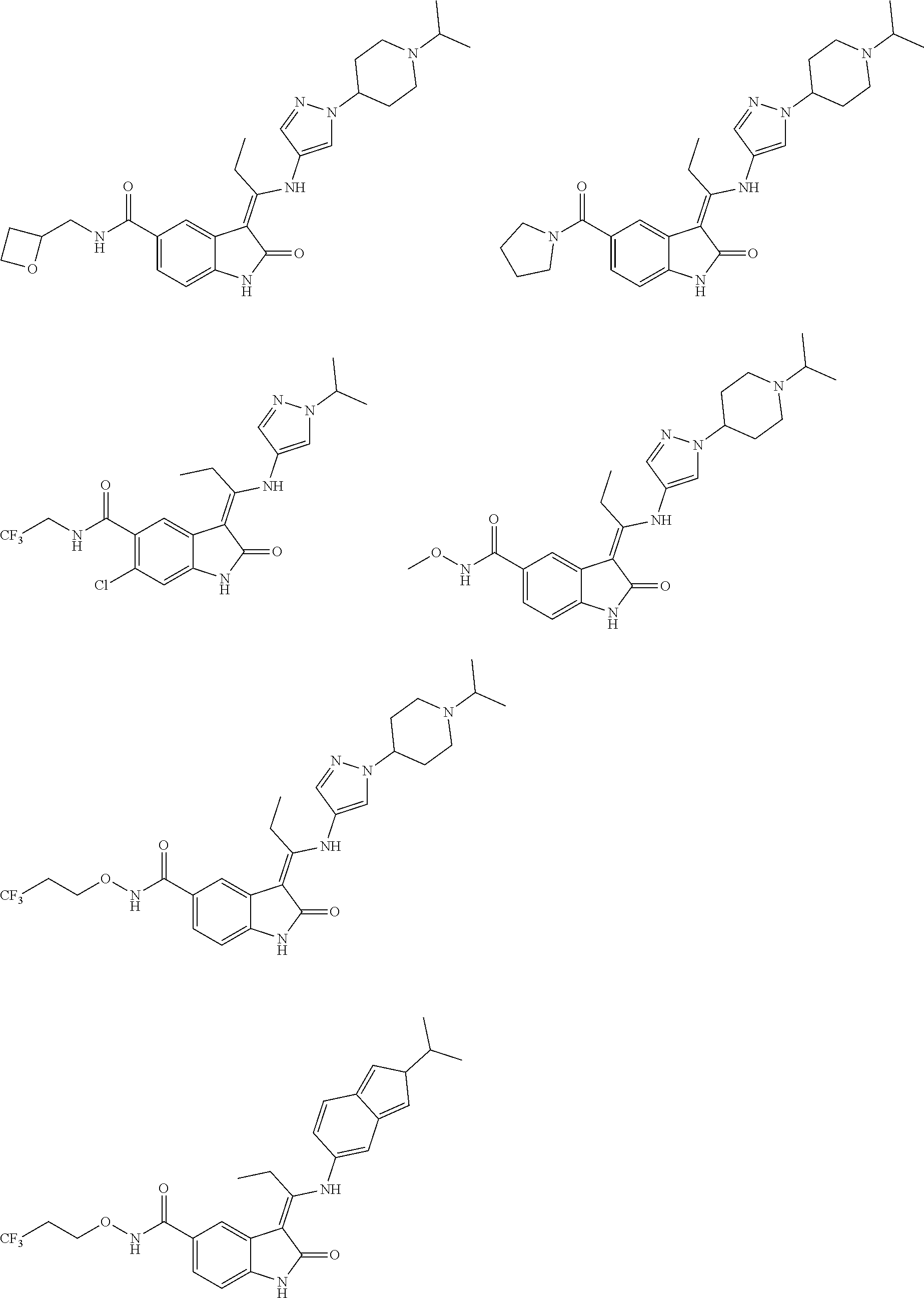 [SMILES: CC/C(Nc1cnn(C2CCN(C(C)C)CC2)c1)=C1/C(=O)Nc2ccc(C(=O)NCC3CCO3)cc21 ; CC/C(Nc1cnn(C2CCN(C(C)C)CC2)c1)=C1/C(=O)Nc2ccc(C(=O)N3CCCC3)cc21 ; CC/C(Nc1cnn(C(C)C)c1)=C1/C(=O)Nc2cc(Cl)c(C(=O)NCC(F)(F)F)cc21 ; CC/C(Nc1cnn(C2CCN(C(C)C)CC2)c1)=C1/C(=O)Nc2ccc(C(=O)NOC)cc21 ; CC/C(Nc1cnn(C2CCN(C(C)C)CC2)c1)=C1/C(=O)Nc2ccc(C(=O)NOCCC(F)(F)F)cc21 ; CC/C(Nc1ccc2c(c1)=CC(C(C)C)C=2)=C1/C(=O)Nc2ccc(C(=O)NOCCC(F)(F)F)cc21] 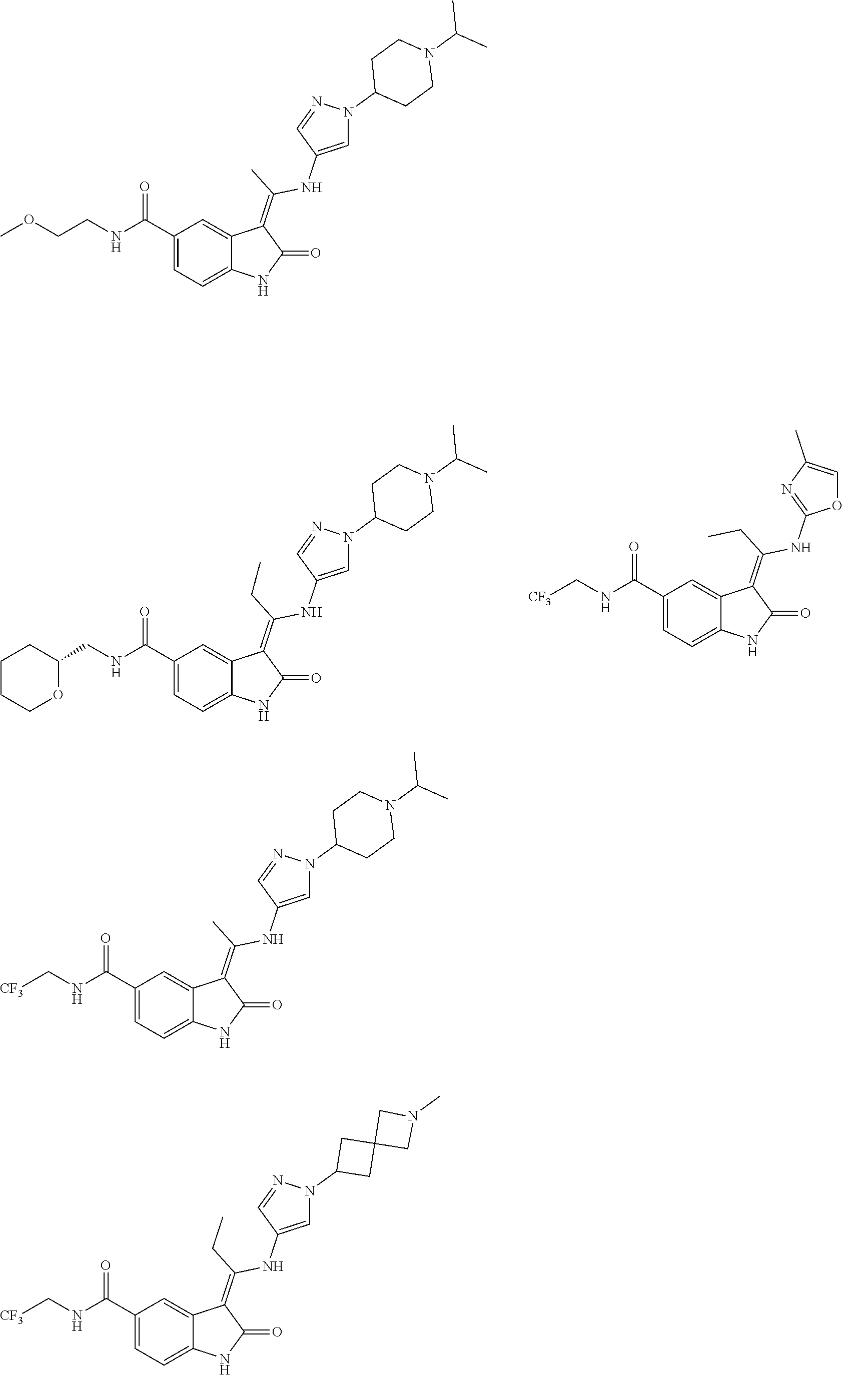 [SMILES: COCCNC(=O)c1ccc2c(c1)/C(=C(\C)Nc1cnn(C3CCN(C(C)C)CC3)c1)C(=O)N2 ; CC/C(Nc1cnn(C2CCN(C(C)C)CC2)c1)=C1/C(=O)Nc2ccc(C(=O)NC[C@H]3CCCCO3)cc21 ; CC/C(Nc1nc(C)co1)=C1/C(=O)Nc2ccc(C(=O)NCC(F)(F)F)cc21 ; C/C(Nc1cnn(C2CCN(C(C)C)CC2)c1)=C1/C(=O)Nc2ccc(C(=O)NCC(F)(F)F)cc21 ; CC/C(Nc1cnn(C2CC3(C2)CN(C)C3)c1)=C1/C(=O)Nc2ccc(C(=O)NCC(F)(F)F)cc21] 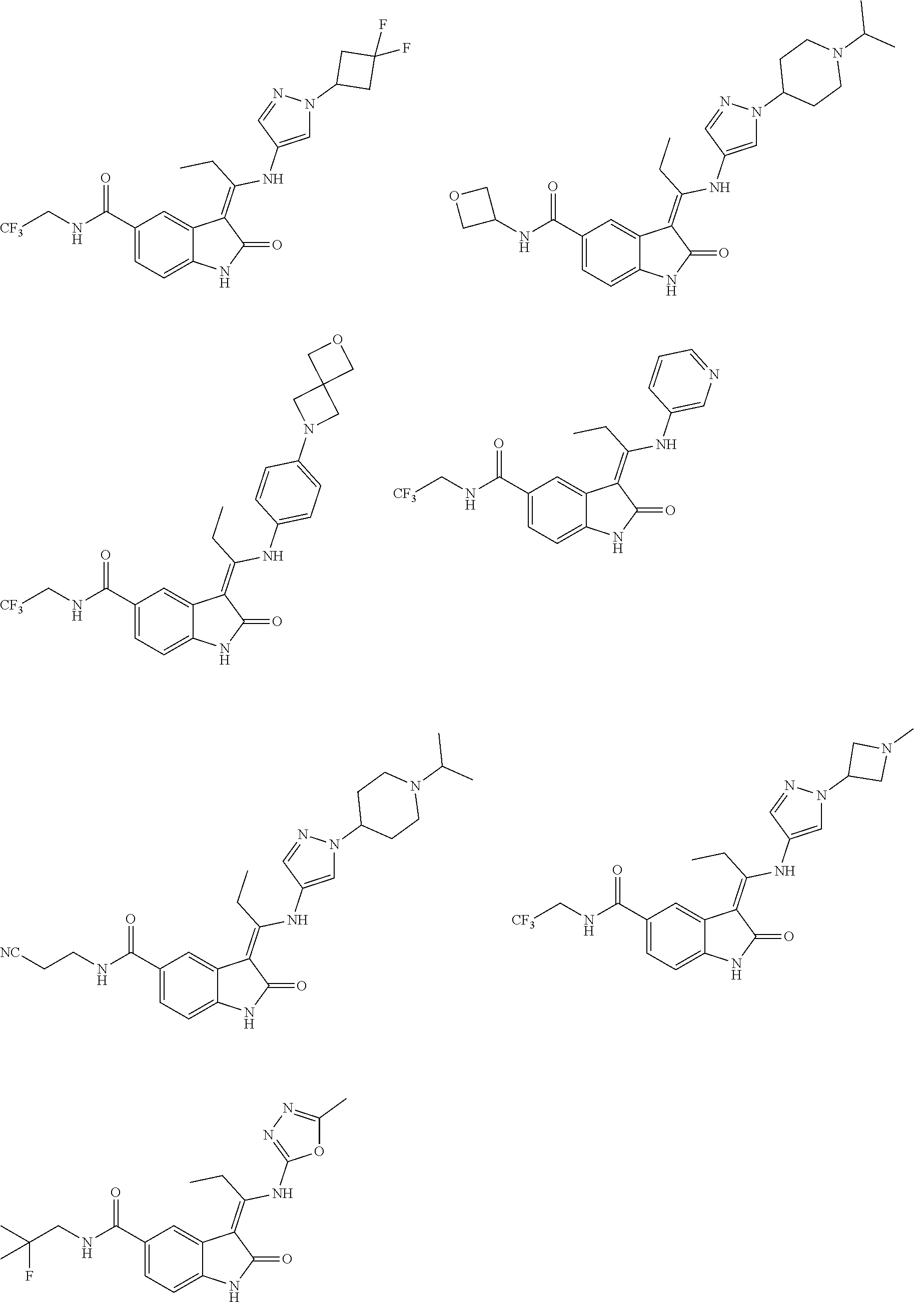 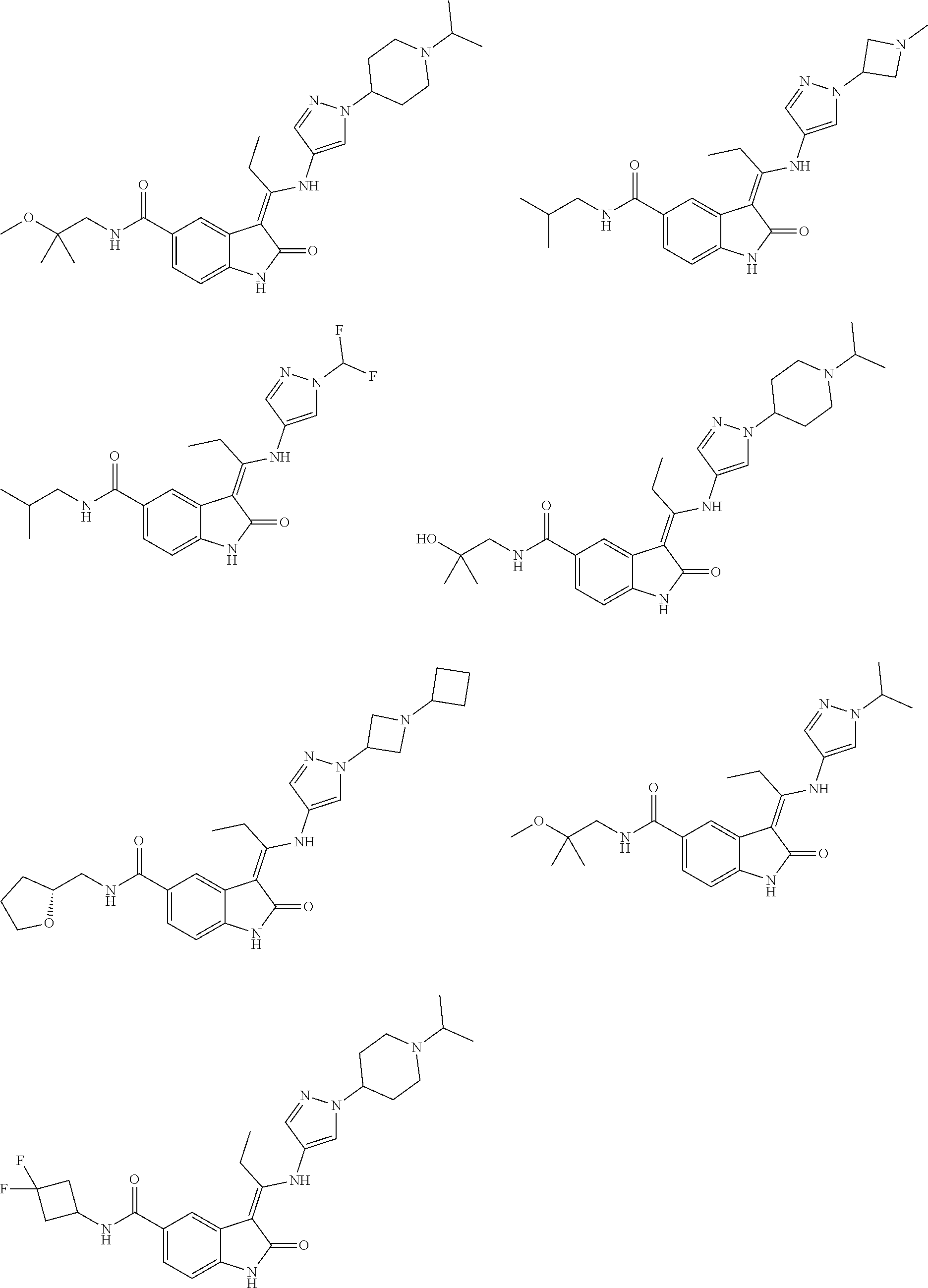 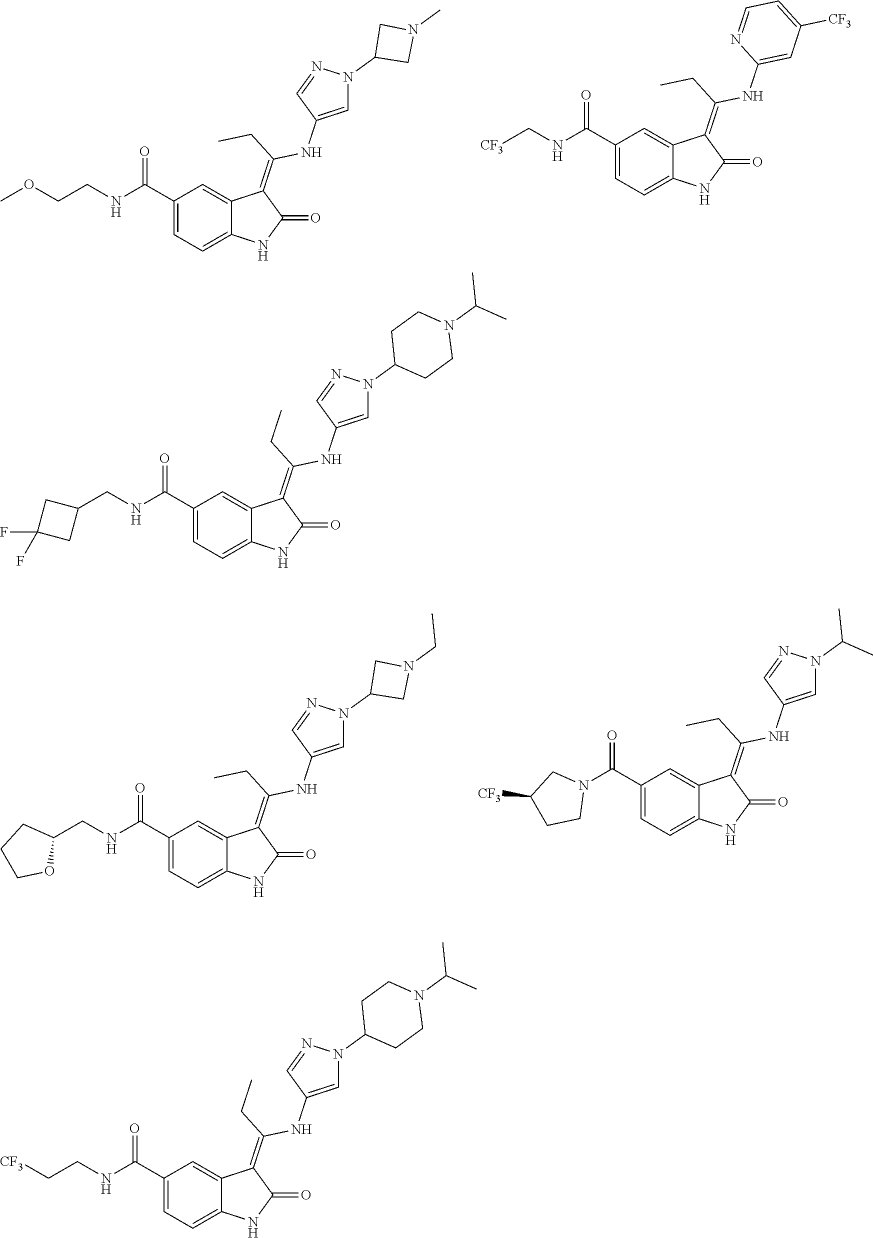 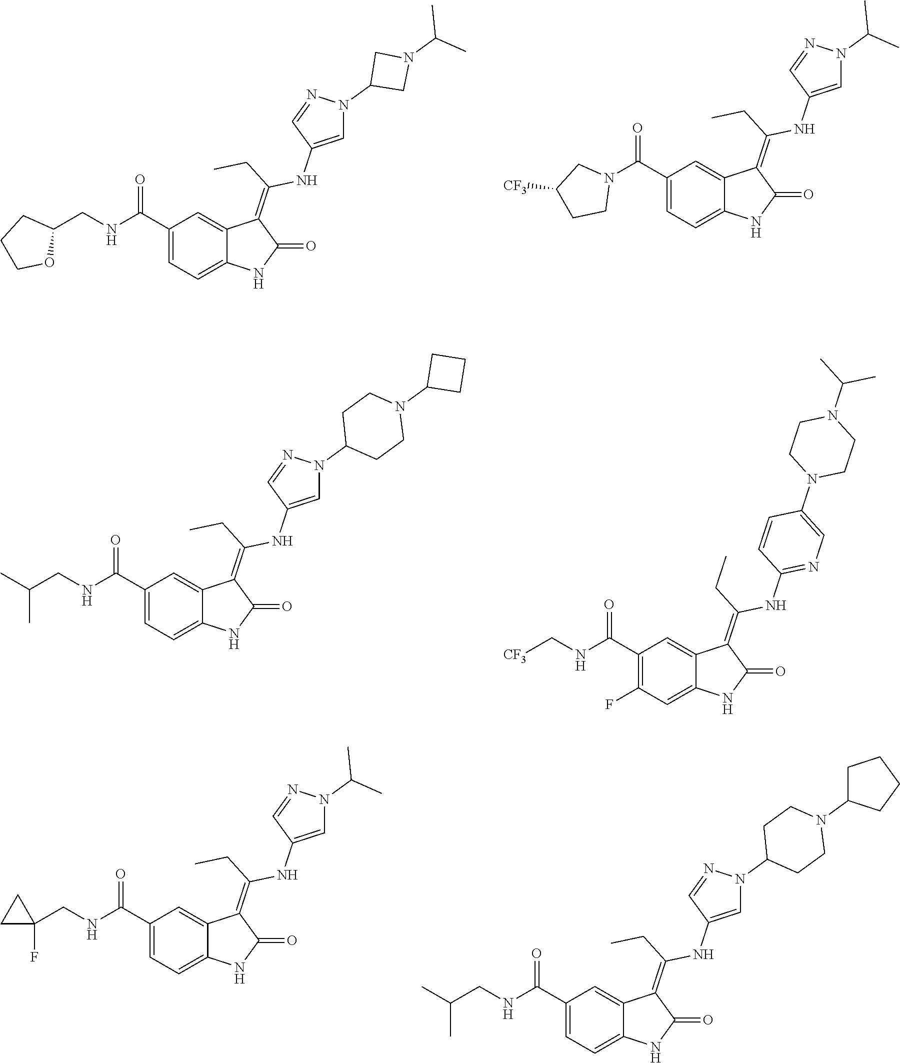 [SMILES: CC/C(Nc1cnn(C2CN(C(C)C)C2)c1)=C1/C(=O)Nc2ccc(C(=O)NC[C@H]3CCCO3)cc21 ; CC/C(Nc1cnn(C(C)C)c1)=C1/C(=O)Nc2ccc(C(=O)N3CC[C@H](C(F)(F)F)C3)cc21 ; CC/C(Nc1cnn(C2CCN(C3CCC3)CC2)c1)=C1/C(=O)Nc2ccc(C(=O)NCC(C)C)cc21 ; CC/C(Nc1ccc(N2CCN(C(C)C)CC2)cn1)=C1/C(=O)Nc2cc(F)c(C(=O)NCC(F)(F)F)cc21 ; CC/C(Nc1cnn(C(C)C)c1)=C1/C(=O)Nc2ccc(C(=O)NCC3(F)CC3)cc21 ; CC/C(Nc1cnn(C2CCN(C3CCCC3)CC2)c1)=C1/C(=O)Nc2ccc(C(=O)NCC(C)C)cc21] 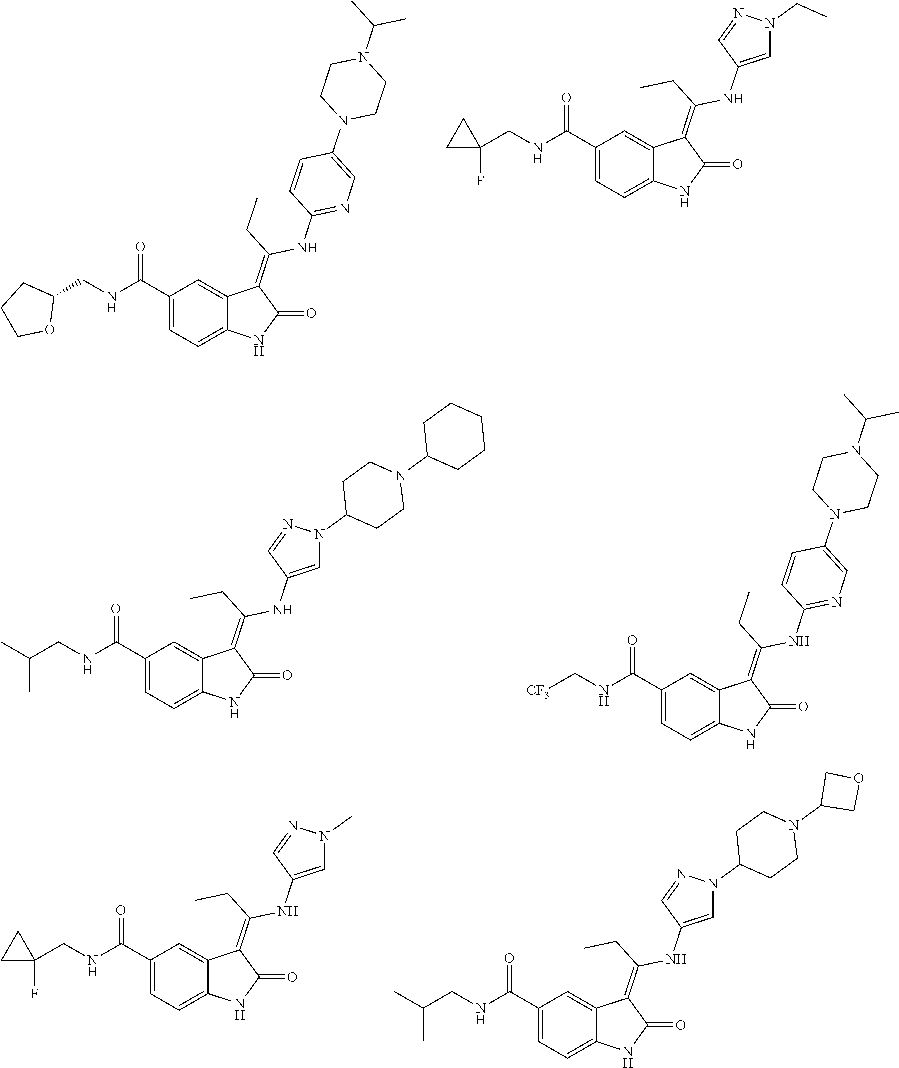 [SMILES: CC/C(Nc1ccc(N2CCN(C(C)C)CC2)cn1)=C1/C(=O)Nc2ccc(C(=O)NC[C@H]3CCCO3)cc21 ; CC/C(Nc1cnn(CC)c1)=C1/C(=O)Nc2ccc(C(=O)NCC3(F)CC3)cc21 ; CC/C(Nc1cnn(C2CCN(C3CCCCC3)CC2)c1)=C1/C(=O)Nc2ccc(C(=O)NCC(C)C)cc21 ; CC/C(Nc1ccc(N2CCN(C(C)C)CC2)cn1)=C1/C(=O)Nc2ccc(C(=O)NCC(F)(F)F)cc21 ; CC/C(Nc1cnn(C)c1)=C1/C(=O)Nc2ccc(C(=O)NCC3(F)CC3)cc21 ; CC/C(Nc1cnn(C2CCN(C3COC3)CC2)c1)=C1/C(=O)Nc2ccc(C(=O)NCC(C)C)cc21] 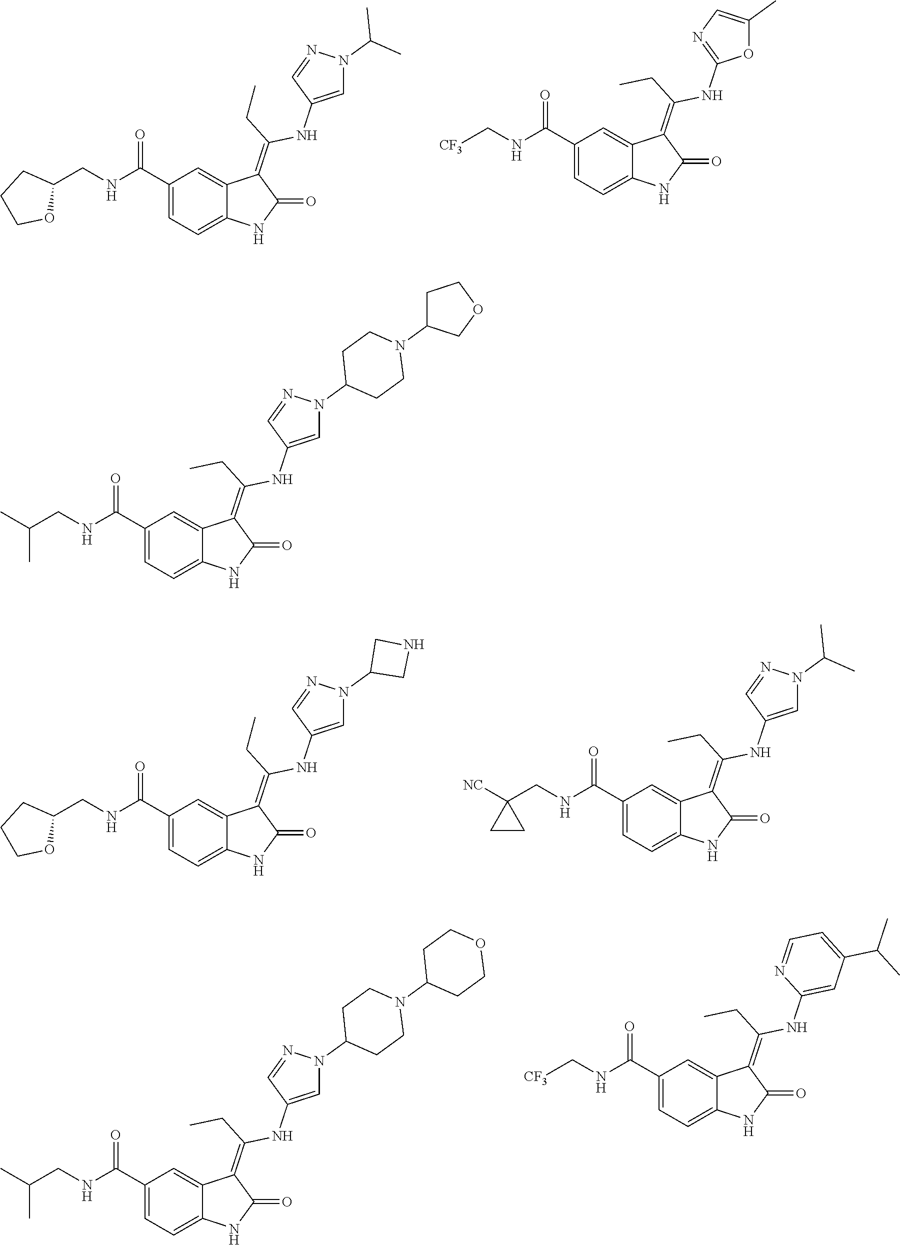 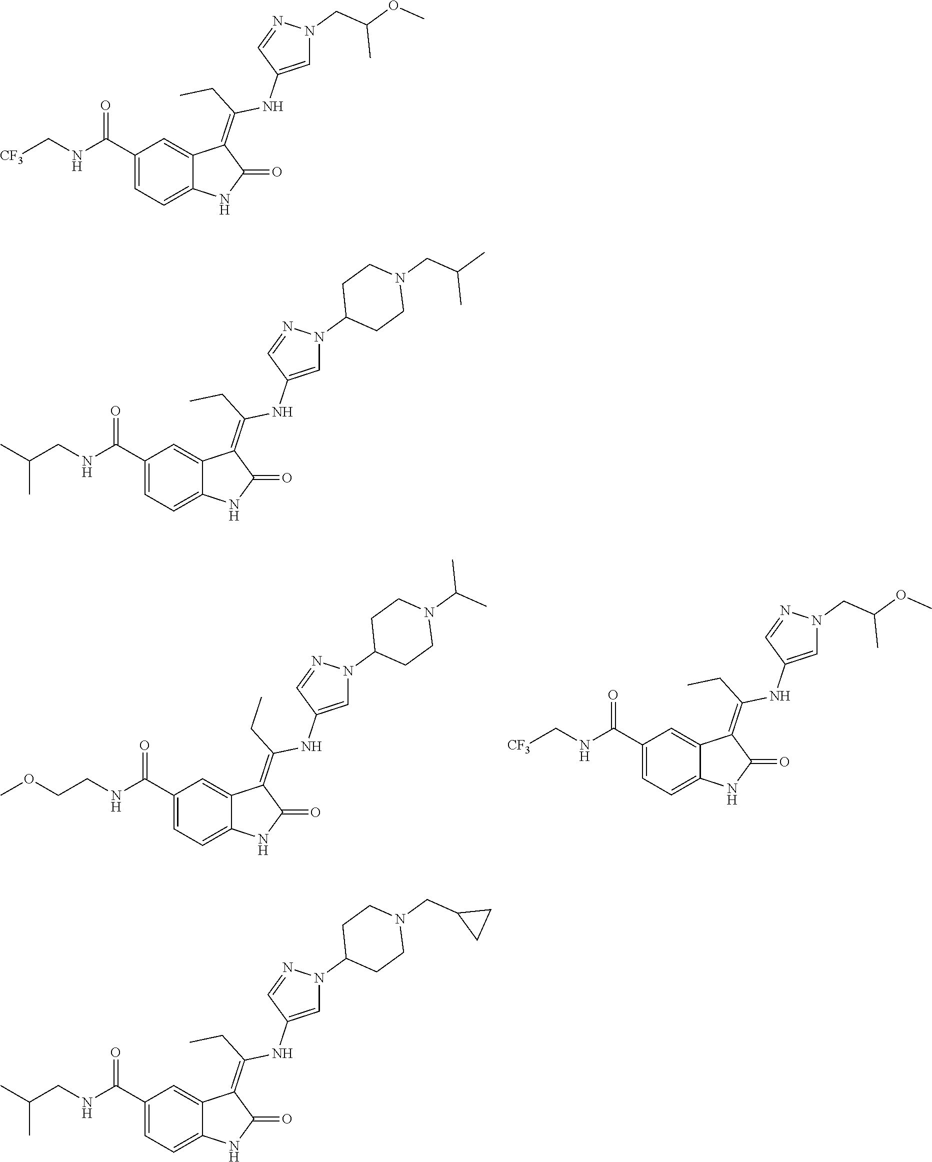 [SMILES: CC/C(Nc1cnn(CC(C)OC)c1)=C1/C(=O)Nc2ccc(C(=O)NCC(F)(F)F)cc21 ; CC/C(Nc1cnn(C2CCN(CC(C)C)CC2)c1)=C1/C(=O)Nc2ccc(C(=O)NCC(C)C)cc21 ; CC/C(Nc1cnn(C2CCN(C(C)C)CC2)c1)=C1/C(=O)Nc2ccc(C(=O)NCCOC)cc21 ; CC/C(Nc1cnn(CC(C)OC)c1)=C1/C(=O)Nc2ccc(C(=O)NCC(F)(F)F)cc21 ; CC/C(Nc1cnn(C2CCN(CC3CC3)CC2)c1)=C1/C(=O)Nc2ccc(C(=O)NCC(C)C)cc21] 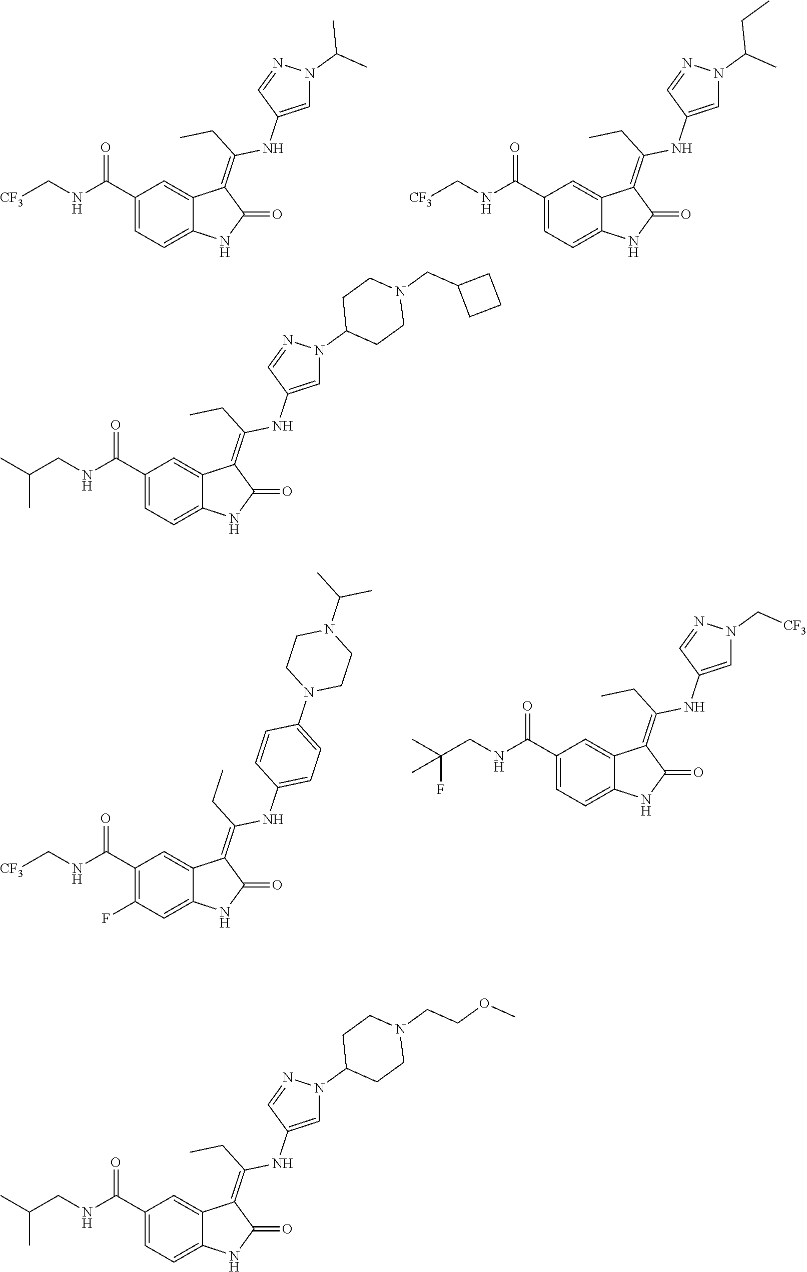 [SMILES: CC/C(Nc1cnn(C(C)C)c1)=C1/C(=O)Nc2ccc(C(=O)NCC(F)(F)F)cc21 ; CC/C(Nc1cnn(C(C)CC)c1)=C1/C(=O)Nc2ccc(C(=O)NCC(F)(F)F)cc21 ; CC/C(Nc1cnn(C2CCN(CC3CCC3)CC2)c1)=C1/C(=O)Nc2ccc(C(=O)NCC(C)C)cc21 ; CC/C(Nc1ccc(N2CCN(C(C)C)CC2)cc1)=C1/C(=O)Nc2cc(F)c(C(=O)NCC(F)(F)F)cc21 ; CC/C(Nc1cnn(CC(F)(F)F)c1)=C1/C(=O)Nc2ccc(C(=O)NCC(C)(C)F)cc21 ; CC/C(Nc1cnn(C2CCN(CCOC)CC2)c1)=C1/C(=O)Nc2ccc(C(=O)NCC(C)C)cc21] 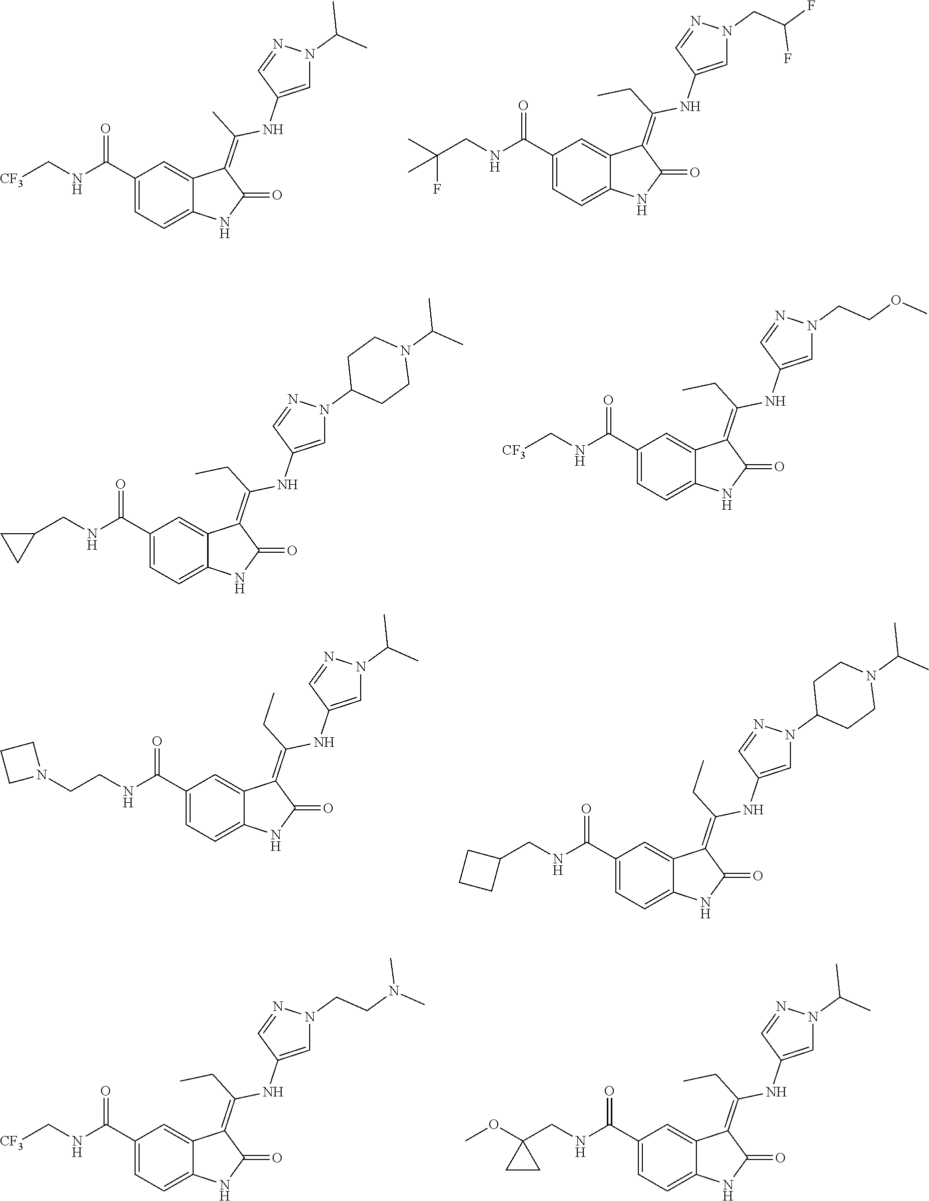 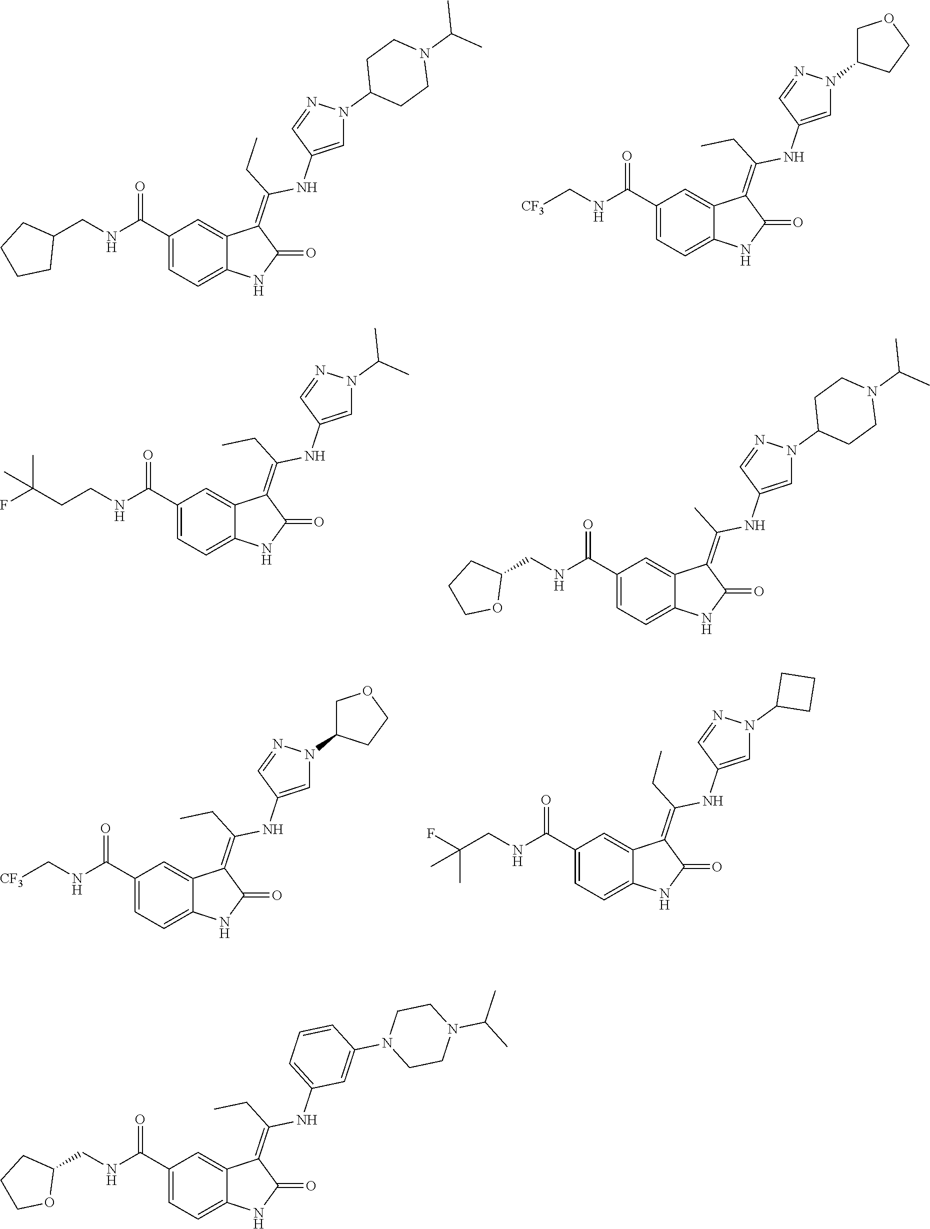 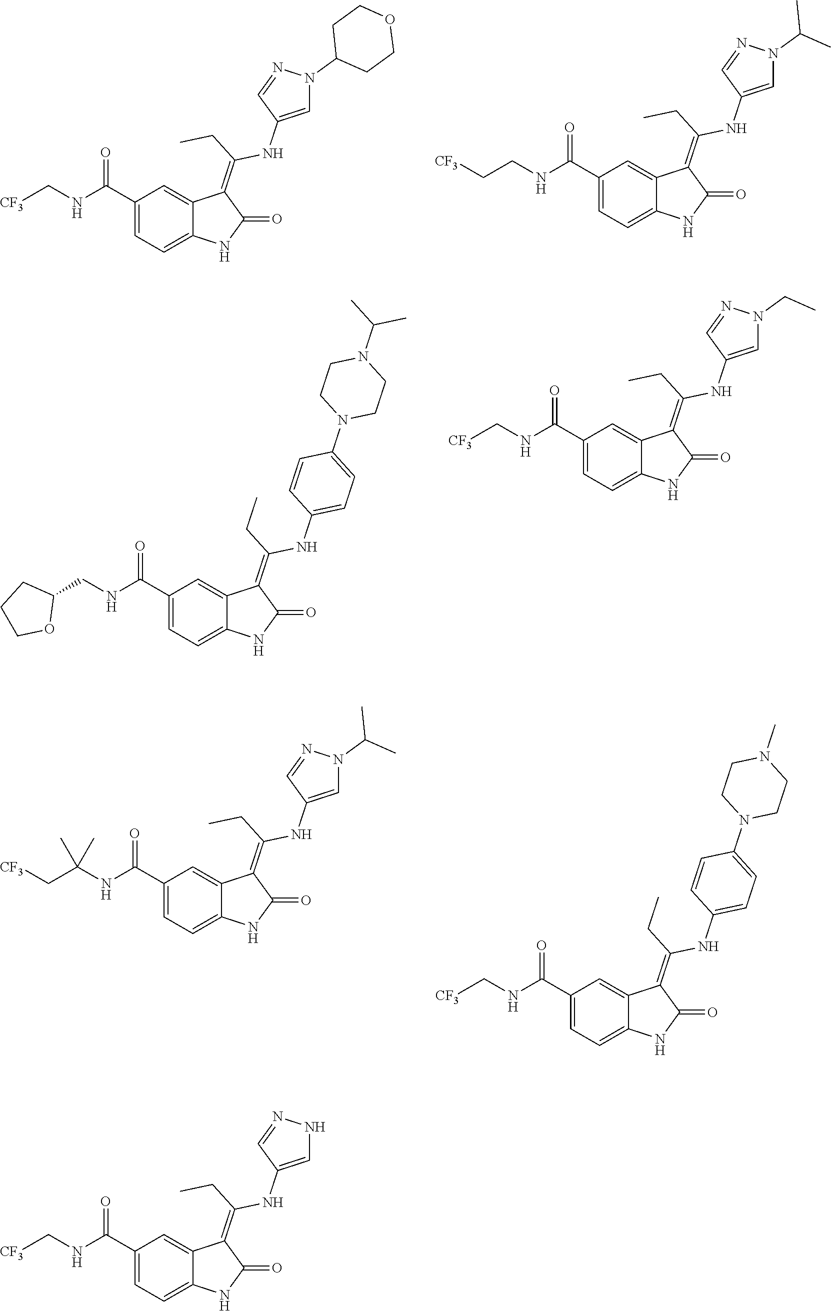 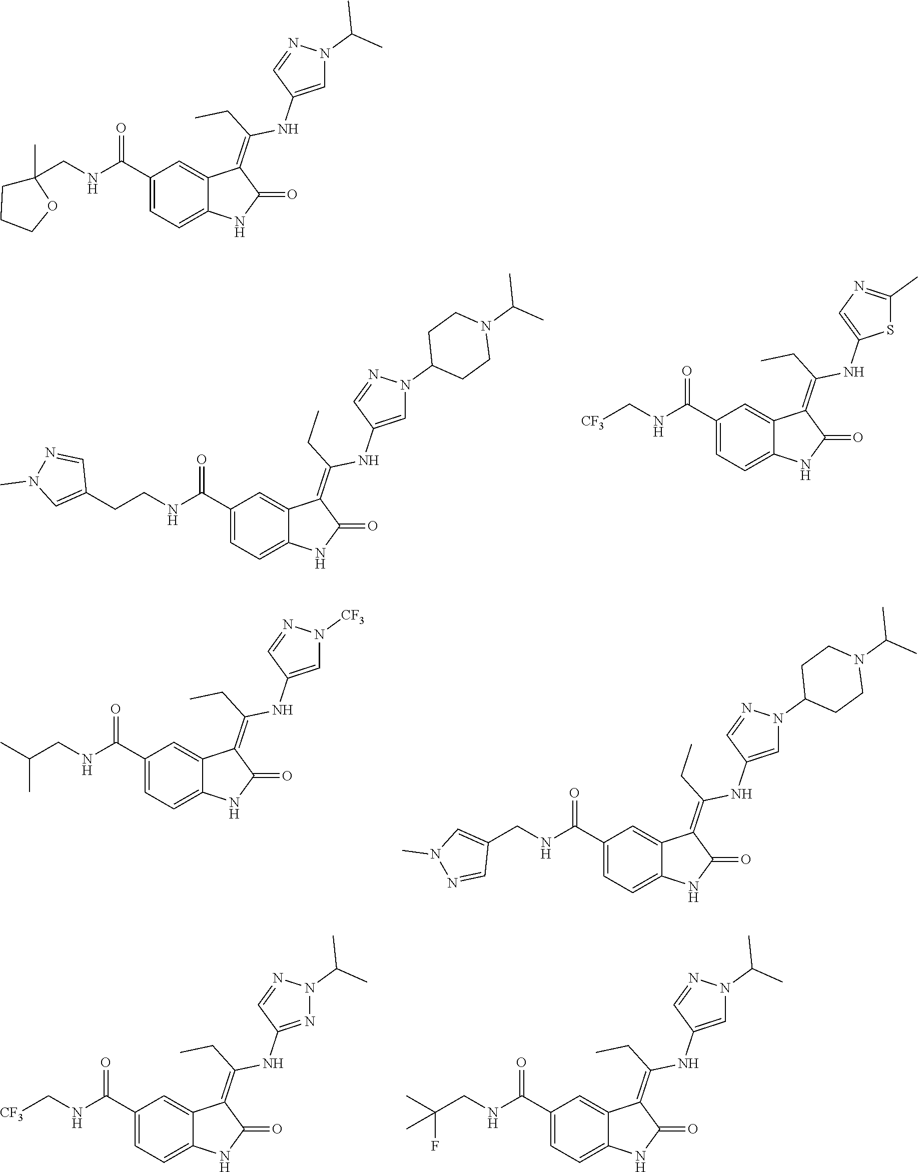 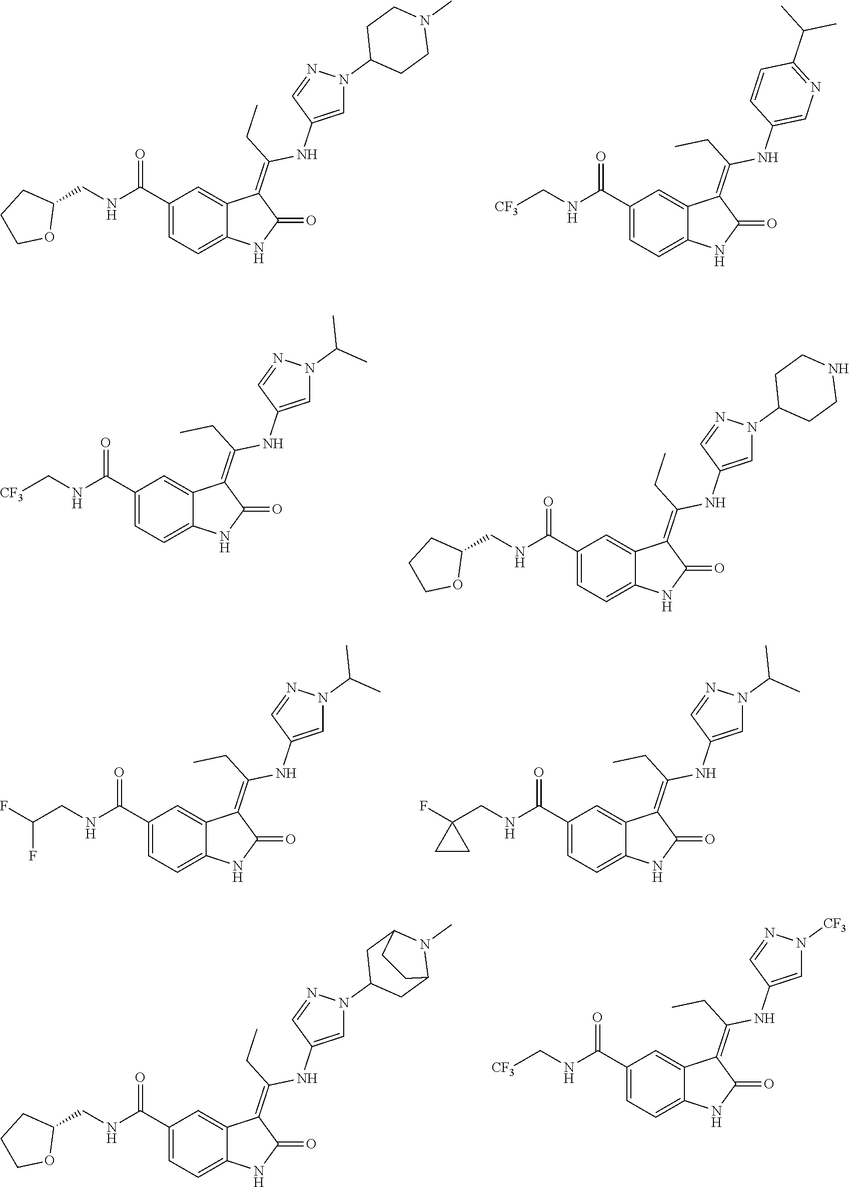 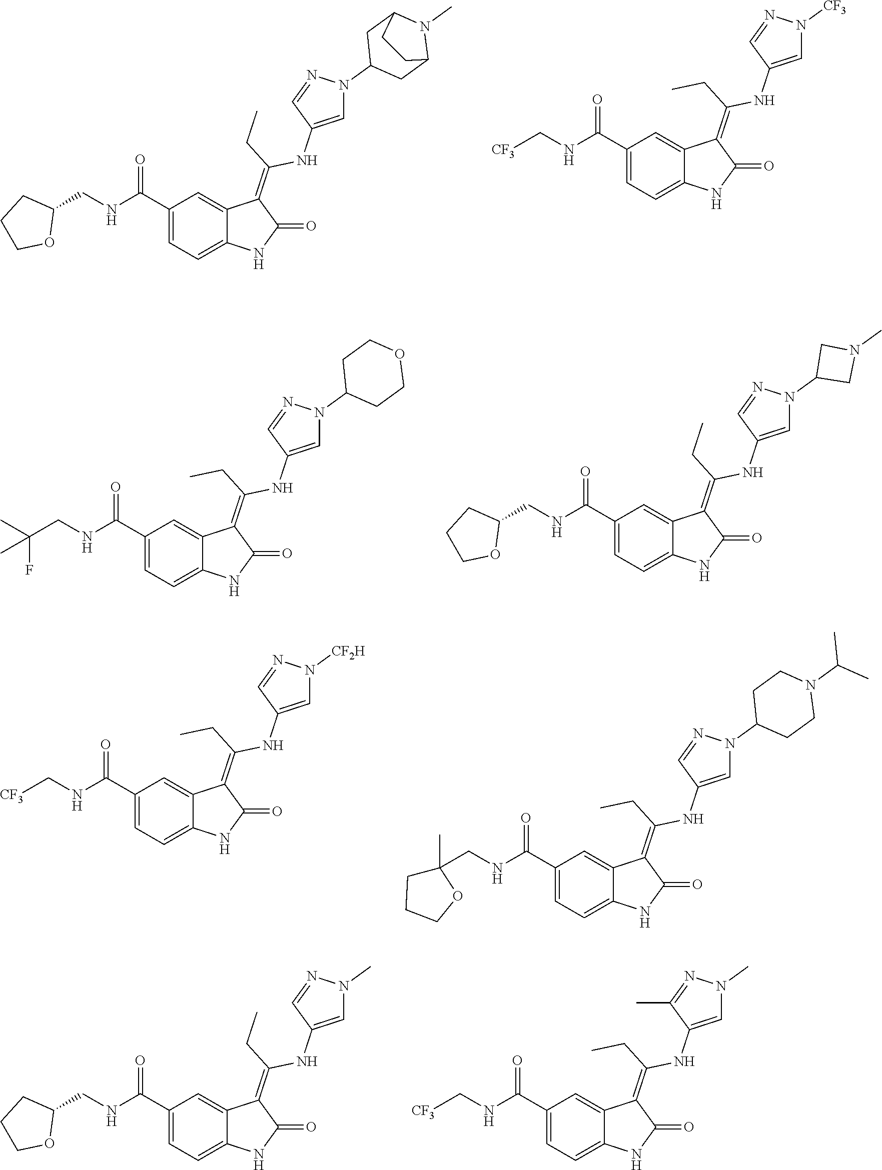 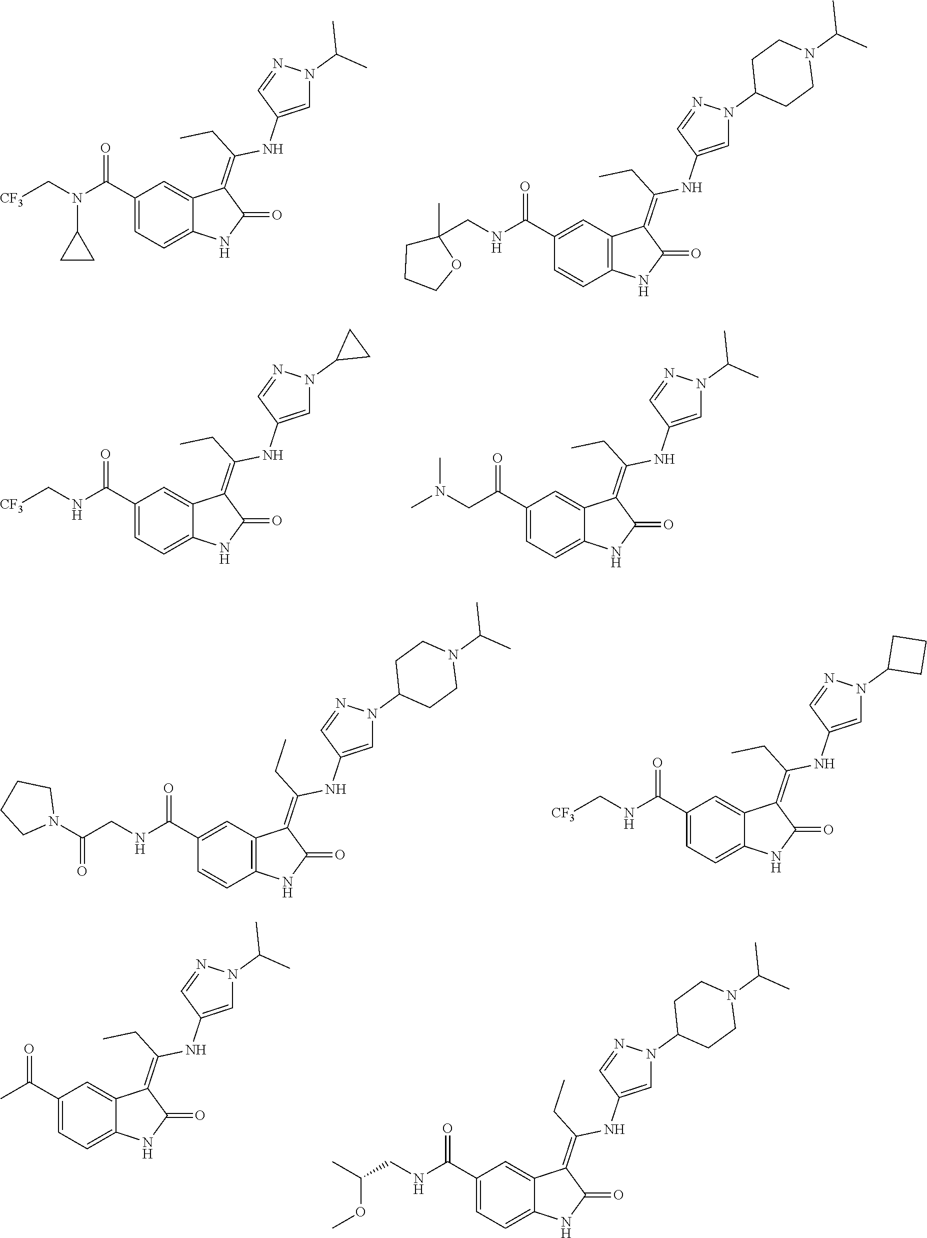 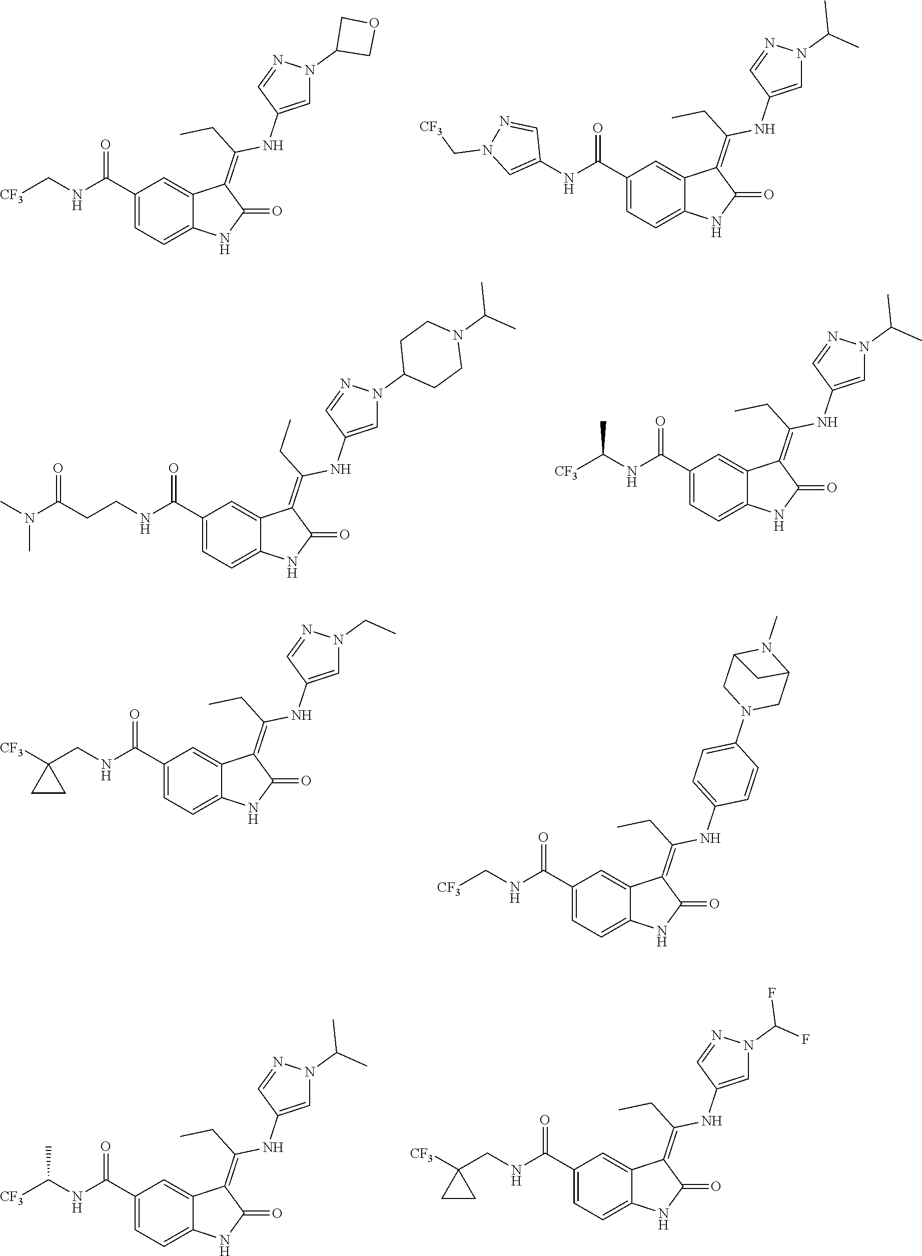 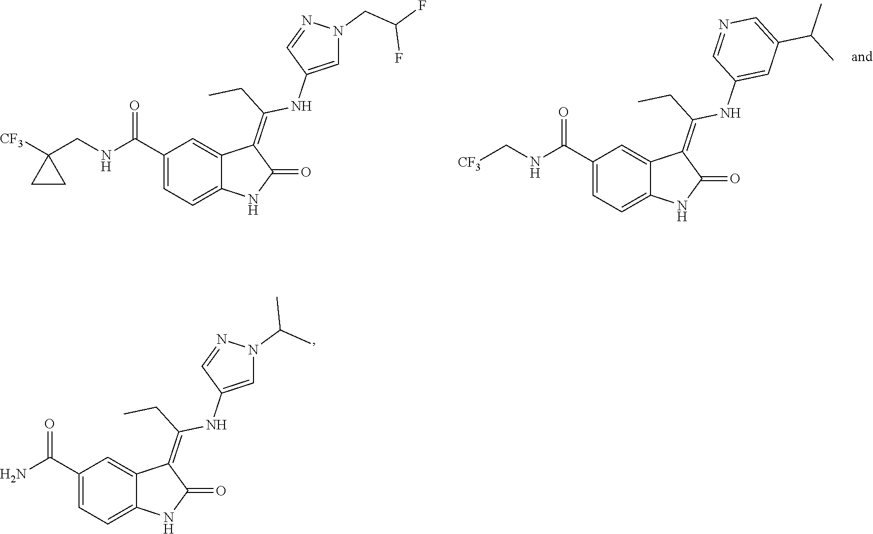 [SMILES: CC/C(Nc1cnn(CC(F)F)c1)=C1/C(=O)Nc2ccc(C(=O)NCC3(C(F)(F)F)CC3)cc21 ; CC/C(Nc1cncc(C(C)C)c1)=C1/C(=O)Nc2ccc(C(=O)NCC(F)(F)F)cc21 ; CC/C(Nc1cnn(C(C)C)c1)=C1/C(=O)Nc2ccc(C(N)=O)cc21]
and pharmaceutically acceptable salts, enantiomers, stereoisomers, or tautomers thereof.

71. A pharmaceutical composition comprising a compound of any one of claims 1-70, or pharmaceutically acceptable salts, enantiomers, stereoisomers, or tautomers thereof, and a pharmaceutically acceptable excipient.

72. A method of treating a tumor in a patient in need thereof, comprising administering to the patient a therapeutically effective amount of a compound of any one of claims 1-70 or pharmaceutically acceptable salts, enantiomers, stereoisomers, or tautomers thereof.

73. A method of treating a cancer in a patient in need thereof, comprising administering to the patient a therapeutically effective amount of a compound of any one of claims 1-70 or pharmaceutically acceptable salts, enantiomers, stereoisomers, or tautomers thereof.

74. The method of claim 73, wherein the cancer is selected from the group consisting of gastrointestinal stromal tumors, esophageal cancer, gastric cancer, melanomas, gliomas, glioblastomas, ovarian cancer, bladder cancer, pancreatic cancer, prostate cancer, lung cancers, breast cancers, renal cancers, hepatic cancers, osteosarcomas, multiple myelomas, leukemias, cervical carcinomas, cancers that are metastatic to bone, papillary thyroid carcinoma, non-small cell lung cancer, and colorectal cancers.

75. The method of claim 73 or claim 74, wherein the cancer is metastatic.

76. A method of treating a disorder selected from the group consisting of gastrointestinal stromal tumors, esophageal cancer, gastric cancer, melanomas, gliomas, glioblastomas, ovarian cancer, bladder cancer, pancreatic cancer, prostate cancer, lung cancers, breast cancers, renal cancers, hepatic cancers, osteosarcomas, multiple myelomas, leukemias, cervical carcinomas, cancers that are metastatic to bone, papillary thyroid carcinoma, non-small cell lung cancer, and colorectal cancers in a patient in need thereof, comprising administering to the patient a therapeutically effective amount of a compound of any one of claims 1-70 or pharmaceutically acceptable salts, enantiomers, stereoisomers, or tautomers thereof.

77. The method of any one of claims 72-76, further comprising administering to the patient one or more additional therapeutic agents.

78. The method of claim 77, wherein the additional therapeutic agent is a RTK pathway inhibitor.

79. The method of claim 78, wherein the RTK pathway inhibitor is selected from the group consisting of a KIT inhibitor, an EGFR inhibitor, a PDGFRα inhibitor, a VEGFR inhibitor, an anti-VEGF therapeutic, a BCR-Abl inhibitor, and an ALK inhibitor.

80. The method of claim 79, wherein the KIT inhibitor is selected from the group consisting of ripretinib, avaprinib, sunitinib, AZD3229, THE-630, and imatinib, and pharmaceutically acceptable salts thereof.

81. The method of claim 79, wherein the EGFR inhibitor is selected from the group consisting of cetuximab, osimertinib, and afatinib, and pharmaceutically acceptable salts thereof.

82. The method of claim 79, wherein the PDGFRα inhibitor is ripretinib, JNJ10198409, or a pharmaceutically acceptable salt thereof.

83. The method of claim 79, wherein the VEGFR inhibitor is selected from the group consisting of regorafenib, axitinib, and pazopanib, and pharmaceutically acceptable salts thereof.

84. The method of claim 79, wherein the anti-VEGF therapeutic is selected from the group consisting of bevacizumab.

85. The method of claim 79, wherein the BCR-Abl inhibitor is imatinib, nilotinib, dasatinib, or a pharmaceutically acceptable salt thereof.

86. The method of claim 79, wherein the ALK inhibitor is selected from the group consisting of lorlatinib, and alectinib, and pharmaceutically acceptable salts thereof.

87. The method of claim 77, wherein the additional therapeutic agent is a MAPKAP pathway inhibitor.

88. The method of claim 87, wherein the MAPKAP pathway inhibitor is selected from the group consisting of a MEK inhibitor, an ERK inhibitor, a RAF inhibitor, and a Ras inhibitor.

89. The method of claim 88, wherein the MEK inhibitor is selected from the group consisting of trametinib, selumetinib, cobimetinib, binimetinib, mirdametinib, and VS-676, and pharmaceutically acceptable salts thereof.

90. The method of claim 88, wherein the ERK inhibitor is selected from the group consisting of ulixertinib, SCH772984, LY3214996, ravoxertinib, VX-11e, ERAS-007, and ASTX-029, and pharmaceutically acceptable salts thereof.

91. The method of claim 88, wherein the RAF inhibitor is selected from the group consisting of LY3009120, LXH254, RAF709, dabrafenib, vemurafenib, belvarafenib, KIN-2787, and VS-6766, and pharmaceutically acceptable salts thereof.

92. The method of claim 88, wherein the Ras inhibitor is selected from the group consisting of AMG-510, MRTX849, GDC-6036, MRTX-1133, RMC-9805, RMC-6291, and RMC-6236, and pharmaceutically acceptable salts thereof.

93. The method of claim 77, wherein the additional therapeutic agent is a PI3K inhibitor or an mTOR inhibitor.

94. The method of claim 93, wherein the PI3K inhibitor is selected from the group consisting of alpelisib, LY294002, and omipalisib, and pharmaceutically acceptable salts thereof.

95. The method of claim 93, wherein the mTOR inhibitor is selected from the group consisting of rapamycin, everolimus, PF04691502, and PP242, and pharmaceutically acceptable salts thereof.

96. The method of claim 77, wherein the additional therapeutic agent is an immunomodulatory agent.

97. The method of claim 96, wherein the immunomodulatory agents is selected from the group consisting of pembrolizumab, nivolumab, atezolizumab, durvalumab, BMS-936559, avelumab, cetuximab, TSR-022, MBG453, relatlimab, LAG525, TSR-033, SGN-40, CP-870,893, RO7009789, Hu5F9-G4, ADU-S100, MK-1454, ASA404, doxorubicin, mitoxanthrone, azacytidine, decitabine, statins, metformin, thalidomide, lenalidomide, pomalidomide, prednisone, dexamethasone, and dostarlimab, and pharmaceutically acceptable salts thereof.

98. The method of claim 77, wherein the additional therapeutic agent is a chemotherapeutic agent.

99. The method of claim 98, wherein the chemotherapeutic agent is a selected from the group consisting of anti-tubulin agents, vinorelbine, DNA-alkylating agents, DNA intercalating agents, 5-fluorouracil, capecitabine, cytarabine, decitabine, 5-azacytidine, gemcitabine, irinotecan, and methotrexate.


SEQUENCE LISTING (6 sequences)

SEQ ID NO: 1 - protein, 283 aa, Homo sapiens
MEPGRGGTETVGKFEFSRKDLIGHGAFAVVFKGRHRAAHDLEVAVKCINKKNLAKSQTLLGKEIKILKELKHENIVALYDFQEMANSVYLVMEYCNGGDLADYLHAMRTLSEDTIRLFLQQIAGAMRLLHSKGIIHRDLKPQNILLSNPAGRRANPNSIRVKIADFGFARYLQSNMMAATLCGSPMYMAPEVIMSQHYDGKADLWSIGTIVYQCLTGKAPFQASSPQDLRLFYEKNKTLVPTIPRETSAPLRQLLLALLQRNHKDRMDFDEFFHHPFLDASPS

SEQ ID NO: 2 - protein, 542 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPEFMEVVGDFEYSKRDLVGHGAFAVVFRGRHRQKTDWEVAIKSINKKNLSKSQILLGKEIKILKELQHENIVALYDVQELPNSVFLVMEYCNGGDLADYLQAKGTLSEDTIRVFLHQIAAAMRILHSKGIIHRDLKPQNILLSYANRRKSSVSGIRIKIADFGFARYLHSNMMAATLCGSPMYMAPEVIMSQHYDAKADLWSIGTVIYQCLVGKPPFQANSPQDLRMFYEKNRSLMPSIPRETSPYLANLLLGLLQRNQKDRMDFEAFFSHPFLEQGPVKKSCPVPVPMYSGSVSGSSCGSSPSCRFASHHHHHH

SEQ ID NO: 3 - protein, 1795 aa, Homo sapiens
MAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRHNQTSLYKKAGTMHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 4 - protein, 10 aa, synthetic construct
  SITE 1: note = Pyroglutamic acid
  SITE 6: note = O-tert-butyl-D-serine
  SITE 10: note = Amidated aza-glycine
EHWSYSLRPG

SEQ ID NO: 5 - protein, 16 aa, synthetic construct
YANWLAASIYLDGKKK

SEQ ID NO: 6 - protein, 15 aa, synthetic construct
RLGRDKYKTLRQIRQ